SEQUENCE LISTING (21 sequences)

SEQ ID NO: 1 - protein, 37 aa, Homo sapiens
ACDTATCVTHRLAGLLSRSGGVVKNNFVPTNVGSKAF

SEQ ID NO: 2 - protein, 37 aa, Homo sapiens
ACNTATCVTHRLAGLLSRSGGMVKSNFVPTNVGSKAF

SEQ ID NO: 3 - protein, 7 aa, Simian virus
PKKKRKV

SEQ ID NO: 4 - protein, 10 aa, Simian virus
PKKKRKVEAS

SEQ ID NO: 5 - protein, 16 aa, Artificial Sequence
  Synthetic
KRPAATKKAGQAKKKK

SEQ ID NO: 6 - protein, 9 aa, Artificial Sequence
  Synthetic
PAAKRVKLD

SEQ ID NO: 7 - protein, 11 aa, Artificial Sequence
  Synthetic
RQRRNELKRSP

SEQ ID NO: 8 - protein, 38 aa, Artificial Sequence
  Synthetic
NQSSNFGPMKGGNFGGRSSGPYGGGGQYFAKPRNQGGY

SEQ ID NO: 9 - protein, 42 aa, Artificial Sequence
  Synthetic
RMRIZFKNKGKDTAELRRRRVEVSVELRKAKKDEQILKRRNV

SEQ ID NO: 10 - protein, 8 aa, Artificial Sequence
  Synthetic
VSRKRPRP

SEQ ID NO: 11 - protein, 8 aa, Artificial Sequence
  Synthetic
PPKKARED

SEQ ID NO: 12 - protein, 8 aa, Homo sapiens
PQPKKKPL

SEQ ID NO: 13 - protein, 12 aa, Murine
SALIKKKKKMAP

SEQ ID NO: 14 - protein, 5 aa, Artificial Sequence
  Synthetic
DRLRR

SEQ ID NO: 15 - protein, 7 aa, Artificial Sequence
  Synthetic
PKQKKRK

SEQ ID NO: 16 - protein, 10 aa, Artificial Sequence
  Synthetic
RKLKKKIKKL

SEQ ID NO: 17 - protein, 10 aa, Murine
REKKKFLKRR

SEQ ID NO: 18 - protein, 20 aa, Homo sapiens
KRKGDEVDGVDEVAKKKSKK

SEQ ID NO: 19 - protein, 17 aa, Homo sapiens
RKCLQAGMNLEARKTKK

SEQ ID NO: 20 - protein, 288 aa, Artificial Sequence
  Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 21 - protein, 183 aa, Artificial Sequence
  Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

(19) United States
(12) Patent Application Publication
(10) Pub. No.: US 2022/0143148 A1
(43) Pub. Date: May 12, 2022

(54) COMPOSITIONS AND METHODS FOR MODULATING CGRP SIGNALING TO REGULATE INTESTINAL INNATE LYMPHOID CELLS

(71) Applicants: The Broad Institute, Inc., Cambridge, MA (US); Massachusetts Institute of Technology, Cambridge, MA (US); The General Hospital Corporation, Boston, MA (US)

(72) Inventors: Aviv Regev, Cambridge, MA (US); Heping Xu, Cambridge, MA (US); Jiarui Ding, Cambridge, MA (US); Ramnik Xavier, Cambridge, MA (US)

(21) Appl. No.: 17/438,972

(22) PCT Filed: Mar. 13, 2020
(86) PCT No.: PCT/US2020/022803

§ 371 Date: Sep. 14, 2021

Related U.S. Application Data
(60) Provisional application No. 62/818,404, filed on Mar. 14, 2019.

(51) Int. Cl. A61K 38/23; A61P 37/00; C12N 15/11; C12N 9/22

(52) U.S. Cl. CPC A61K 38/23; A61P 37/00; C12N 2310/20; C12N 9/22; C12N 15/111

(57) ABSTRACT

The present invention provides novel compositions and methods based on the discovery of the mechanisms and gene expression programs associated with homeostatic ILC2s and proinflammatory ILC2s that drive tissue inflammation. Immune signaling abnormalities in the small intestine can trigger chronic type 2 inflammation. Applicants analyzed 58,067 immune cells from the mouse small intestine by single-cell RNA-seq at steady state and after induction of a type 2 inflammatory reaction to ovalbumin. Cell type composition and cell programs shifted in response to inflammation, especially in ILC2s. A key transcript in the inflammation-induced program in intestinal KLRG1+ILC2s was exon 5 of Calca, encoding the alpha-calcitonin gene-related peptide (a-CGRP). a-CGRP antagonized IL-25-induced activation of intestinal ILC2s and reduced their frequency in an ovalbumin reaction model. α-CGRP activated a cAMP response, which suppressed ILC2 proliferation. In homeostasis, α-CGRP was expressed by two subsets of ChAT+ enteric neurons, and genetic perturbation of α-CGRP increased the proportion of intestinal ILC2s and of Tuft cells. The results demonstrate that a-CGRP-mediated neuronal signaling suppresses ILC2 expansion and maintains type 2 immunity homeostasis.

### CROSS-REFERENCE TO RELATED APPLICATIONS

This application claims the benefit of U.S. Provisional Application No. 62/818,404, filed Mar. 14, 2019. The entire contents of the above-identified application are hereby fully incorporated herein by reference.

### REFERENCE TO AN ELECTRONIC SEQUENCE LISTING

The contents of the electronic sequence listing (BROD_4040_ST25.txt”; Size is 9,000 Kilobytes and it was created on Mar. 13, 2020) is herein incorporated by reference in its entirety.

### TECHNICAL FIELD

The subject matter disclosed herein is generally directed to compositions and methods for modulating intestinal ILC2 cells and responses by targeting CGRP signaling.

### BACKGROUND

The immune system in the small intestine is comprised of a complex network of innate and adaptive components that sense and respond antigens from the diet, commensal microbiota and pathogens. Dysregulated immune reactions often lead to chronic inflammatory responses, including type 2 inflammation (Gieseck et al., 2018; Hammad and Lambrecht, 2015; Pulendran and Artis, 2012), which in turn plays a key underlying role in several unrelenting inflammatory diseases, including food allergy (Locksley, 2010; Tordesillas et al., 2017).

Type 2 inflammation is characterized by the production of the cytokines interleukin-4 (IL-4), IL-5, IL-13 and IgE antibody, and tightly regulated and coordinated responses across cell types, including T helper 2 (Th2) cells, B cells, dendritic cells (DCs) and mast cells. In particular, ILC2s have emerged as key regulators of tissue homeostasis and type 2 inflammation (Artis and Spits, 2015; Kotas and Locksley, 2018), and form the prominent source of type 2 cytokines at its early stages (Molofsky et al., 2015; Neill et al., 2010; Price et al., 2010). While the core transcriptional program of ILCs maintains their cellular identity and homeostatic type 2 cytokine competency (Ricardo-Gonzalez et al., 2018), their activity in the intestines is shaped by tissue-specific signals, including activation by IL-25 produced by Tuft cells (von Moltke et al., 2016), and suppression by micronutrients (Spencer et al., 2014).

Despite these substantial advances, our understanding of cellular participants in type 2 inflammation, the mechanisms that maintain homeostasis and induce inflammation, and which additional cell types, besides immune cells, may participate in this cellular circuit in the small intestines is incomplete. Single-cell RNA-seq (scRNA-seq) can dissect cellular diversity on a large scale (Tanay and Regev, 2017; Wagner et al., 2016) and identify cell states of individual cell types in response to different stimuli (Bielecki et al., 2018; Haber et al., 2017). For example, scRNA-seq of lung ILCs recently revealed that neuronal-derived Neuromedin U (NMU) amplifies ILC2 activity in allergic inflammation (Wallrapp et al., 2017), and the same neuron-immune circuit was also shown to induce activation of ILC2s in the small intestine (Cardoso et al., 2017; Klose et al., 2017). Given the increased prevalence and epidemic rise in allergy and asthma in the last two decades, identifying the molecular pathways that regulate ILC2s during allergic responses is an important area of inquiry.

Citation or identification of any document in this application is not an admission that such document is available as prior art to the present invention.

### SUMMARY

In one aspect, the present invention provides for a method of maintaining or inducing homeostasis of intestinal ILC2 cells in a subject at risk for or having aberrant activation and expansion of the intestinal ILC2 cells comprising administering CGRP to the subject. In certain embodiments, the aberrant activation and expansion of the intestinal ILC2 cells is induced by IL-25. In certain embodiments, the CGRP is administered intravenously, intraperitoneally, intragastrically, or orally. In certain embodiments, the subject has an allergy or history of allergic symptoms. In certain embodiments, the allergy is a food allergy. In certain embodiments, the allergy is caused by an allergen that induces epithelial cells in the gut to release IL-25. In certain embodiments, CGRP is administered after the subject has contacted or ingested an allergen. In certain embodiments, the CGRP is administered before an inflammatory response. In certain embodiments, CGRP is administered upon detecting an inflammatory response. In certain embodiments, the subject does not have an infection, such as a helminth infection. In certain embodiments, the method further comprises administering to the gut of the subject one or more agents capable of modulating expression, activity or function of one or more genes selected from the group consisting of: Gpr65, Pdcd1, Crem, Egln3, Adora2a, Rgs2, Gna15, Adrb2, Gadd45a, Areg, Hif1a, Dusp1, Pde4b, Cdkn1a, Akap12 and Il5. In certain embodiments, CGRP induces expression of Gpr65, Pdcd1, Crem, Egln3, Adora2a, Rgs2, Gna15, Adrb2, Gadd45a, Areg, Hif1a, Dusp1, Pde4b, Cdkn1a, Akap12 and Il5 to maintain homeostasis of intestinal ILC2 cells. Thus, in intestinal ILC2s, treatment with CGRP increases the expression of one or more of Gpr65, Pdcd1, Crem, Egln3, Adora2a, Rgs2, Gna15, Adrb2, Gadd45a, Areg, Hif1a, Dusp1, Pde4b, Cdkn1a, Akap12 and Il5, and a combination treatment may provide for prevention of aberrant intestinal ILC2 inflammatory responses. In certain embodiments, the method comprises activating a cAMP response module, wherein the cAMP module comprises one or more genes selected from the group consisting of Adrb2, Adora2a, Pde4b, Akap12, Areg, Crem and Il5. In certain embodiments, the one or more agents comprises an adenylate cyclase activator. In certain embodiments, the agent is forskolin. In certain embodiments, the one or more agents comprises an agonist of PD-1. In certain embodiments, the one or more agents comprises an agonist of GPR65.

In another aspect, the present invention provides for a method of maintaining or inducing homeostasis of intestinal ILC2 cells in a subject at risk for or having aberrant activation and expansion of the intestinal ILC2 cells comprising administering to the gut of the subject one or more agents capable of modulating expression, activity or function of one or more genes selected from the group consisting of Gpr65, Pdcd1, Crem, Egln3, Adora2a, Rgs2, Gna15, Adrb2, Gadd45a, Areg, Hif1a, Dusp1, Pde4b, Cdkn1a, Akap12 and Il5. In certain embodiments, CGRP induces expression of Gpr65, Pdcd1, Crem, Egln3, Adora2a, Rgs2, Gna15, Adrb2, Gadd45a, Areg, Hif1a, Dusp1, Pde4b, Cdkn1a, Akap12 and Il5 to maintain homeostasis of intestinal ILC2 cells. Thus, in intestinal ILC2s, increasing the expression, activity or function of one or more of Gpr65, Pdcd1, Crem, Egln3, Adora2a, Rgs2, Gna15, Adrb2, Gadd45a, Areg, Hif1a, Dusp1, Pde4b, Cdkn1a, Akap12 and Il5 may provide for prevention of aberrant intestinal ILC2 inflammatory responses. In certain embodiments, the method comprises activating a cAMP response module, wherein the cAMP module comprises one or more genes selected from the group consisting of Adrb2, Adora2a, Pde4b, Akap12, Areg, Crem and Il5. In certain embodiments, the one or more agents comprises an adenylate cyclase activator. In certain embodiments, the agent is forskolin. In certain embodiments, the one or more agents comprises an agonist of PD-1. In certain embodiments, the one or more agents comprises an agonist of GPR65. In certain embodiments, the subject has an allergy or history of allergic symptoms. In certain embodiments, the allergy is a food allergy. In certain embodiments, the allergy is caused by an allergen that induces epithelial cells in the gut to release IL-25. In certain embodiments, the subject does not have an infection, such as a helminth infection.

In another aspect, the present invention provides for a method of modulating an ILC2 inflammatory response comprising administering to a population of cells comprising ILC2s, mast cells, Th2 cells and/or fibroblasts one or more agents capable of modulating expression, activity or function of one or more biological programs characterized by ILC Topic 2, myeloid cell Topic 1, T cell Topic 5 or stromal cell Topic 4, wherein ILC Topic 2 comprises one or more genes or polypeptides selected from the group consisting of Calca, Hs3st1, Areg, Il13, Il4, Ccl1, Hes1, Il17rb, Lgals7, Homer2, Il5, Gata3, Deptor, Ptpn13, Ly6a, Hba-a1, Kcnn4, Ccr4, Rxrg, Sub1, 1700061F12Rik, Cntnap2, AA467197, Ptgir, Il10, Nfkb1, Lmo4, Pparg, Plaur, Il9r, Serpine1, Scel, Bmp7, Neb, Sox8, Lpcat2, Samsn1, Alox5, Gpr65, Abhd17c, Gm20186, Gm973, Epas1, Ccr8, D430036J16Rik, Cd6, Stxbp6, 9230102O04Rik, Furin and Klf5, wherein myeloid cell Topic 1 comprises one or more genes or polypeptides selected from the group consisting of Cpa3, Cma1, Mcpt4, Tpsb2, Fcer1a, Hs3st1, Gata2, Cited4, Cyp11a1, Tph1, Furin, Rab27b, Slc45a3, Ccl1, Il13, Il1rl1, Itga2b, Cited2, Fam110c, Creb3l1, Rgs13, Tpsab1, Cyp26a1, Serpinb1a, Slc18a2, Gmpr, Rprm, Ero1l, Il4, Cd200r3, Glul, Kit, Lat, Alox5, Gchfr, mt-Atp6, Lat2, Prss34, Poln, Klk8, 4932438H23Rik, Slc6a13, Avil, Socs2, Smco4, Ier3, Lxn, Gpr171, Adk and Gata1, wherein T cell Topic 5 comprises one or more genes or polypeptides selected from the group consisting of 1700061F12Rik, Il13, Scin, Lgmn, Hlf, Smco4, Npnt, Il17rb, Deptor, Gata3, Gm2a, Il6, Il17a, Ltb4r1, Fgl2, Areg, Fbxl21, AA467197, Il1rl1, Me1, Gm5544, Tmem159, Rasgrp4, 1700012B07Rik, 1700113H08Rik, St6galnac5, Il4, Chdh, Slco2b1, Ccr9, Epas1, Grp, Lztfl1, Gm10369, Kif19a, Tenm4, Serpinf1, Gnb2, Ubox5, Plcl1, Rab31, Ffar2, Slx1b, Asb2, Zfp85, Tmsb4x, Hdc, Pxdc1, Heatr1 and Lgals7, wherein stromal cell Topic 4 comprises one or more genes or polypeptides selected from the group consisting of Ccl21a, Cxcl13, Clu, Ccl19, Acta2, Mfge8, Apoe, Tagln, Cxcl1, Cilp, Ccl2, Il33, Cxcl12, Actg2, Serpina3n, Ccl7, Bst1, Serpina1a, Fmod, Grem1, Serpina1b, Slc36a2, Cnn1, Myh11, Art2b, Actc1, AI838599, Serpina1c, Cr2, Gxylt2, Crym, Dclk1, Serpina1d, Myl9, Parm1, Gm16685, Postn, Chrdl1, Colq, Csn2, Prss12, H2-M2, Trf, Sostdc1, Dsc3, Ctgf, Thbs4, Pcdh15, Rtn4r and A230065H16Rik. In certain embodiments, the one or more biological programs are suppressed, whereby an ILC2 inflammatory response is decreased. In certain embodiments, the one or more agents modulate the expression, activity or function of one or more genes or polypeptides in ILC Topic 2, myeloid cell Topic 1, T cell Topic 5 or stromal cell Topic 4. In certain embodiments, the population of cells is present in the gut of a subject in need thereof. In certain embodiments, the population of cells is an in vitro population of cells. In certain embodiments, the population of cells is an intestinal organoid.

In certain embodiments, the one or more agents comprise an antibody, small molecule, small molecule degrader, genetic modifying agent, antibody-like protein scaffold, aptamer, protein, or any combination thereof. In certain embodiments, the genetic modifying agent comprises a CRISPR system, RNAi system, a zinc finger nuclease system, a TALE, or a meganuclease. In certain embodiments, the CRISPR system is a Class I or Class II CRISPR system. In certain embodiments, the Class II system comprises a Class 2, Type II Cas polypeptide. In certain embodiments, the Type II Cas is a Cas9. In certain embodiments, the Class II system comprises a Class 2, Type V Cas polypeptide. In certain embodiments, the Type V Cas is Cas12a or Cas12b. In certain embodiments, the Class II system comprises a Class 2, Type VI Cas polypeptide. In certain embodiments, the Type VI Cas is Cas13a, Cas13b, Cas13c or Cas13d. In certain embodiments, the CRISPR system comprises a dCas fused or otherwise linked to a nucleotide deaminase. In certain embodiments, the nucleotide deaminase is a cytidine deaminase or an adenosine deaminase. In certain embodiments, the dCas is a dCas9, dCas12, or dCas13.

In another aspect, the present invention provides for a method of quantitating a type 2 immune response comprising determining the ILC2 frequency, wherein increased frequency of ILC2s as compared to a control frequency is associated with an increased type 2 immune response. In certain embodiments, the method further comprises determining the frequency of one or more cells selected from the group consisting of mast cells, macrophages, neutrophils, and CD11b+CD103+ dendritic cells, wherein increased frequency of mast cells, macrophages and/or neutrophils, and/or decreased frequency of CD11b+CD103+ dendritic cells as compared to a control frequency is associated with an increased type 2 immune response.

In another aspect, the present invention provides for a method of quantitating a type 2 immune response comprising determining the expression of one or more genes selected from Table 3 or determining the frequency of the cell types expressing the one or more genes selected from Table 3, wherein changes in expression or frequency according to Table 3 is associated with an increased type 2 immune response. In certain embodiments, the method comprises determining the expression of: one or more genes in ILC2s selected from the group consisting of: Hes1, Il13, Lif, Areg and Il4; one or more genes in mast cells selected from the group consisting of: Mcpt4, Tph1, Mcpt1, Cma1, and Furin; one or more genes in macrophages selected from the group consisting of: Irf7, Isg15, Irf8, Irf1, Ccl7, Ccl2, Cxcl12, Pf4 and Ccl24; and/or one or more genes in plasma cells selected from the group consisting of: Ifi27, Ifitm3, Ifnar1, Ighg1 and Ighe, wherein increased or decreased expression in the cell type according to FIG. 9B or 9C is associated with an increased type 2 immune response.

In another aspect, the present invention provides for a method of quantitating a type 2 immune response comprising determining the frequency of IL-33⁺PDPN⁺ fibroblasts in a subject having an allergy, wherein increased frequency of IL-33⁺PDPN⁺ fibroblasts is associated with an increased type 2 immune response.

In certain embodiments, CGRP is a polypeptide comprising the amino acid sequence of SEQ ID NO: 1. In certain embodiments, the CGRP sequence is modified to increase stability of the polypeptide. In certain embodiments, the intestinal ILCs are KLRG^{Hi} ST2⁻ ILCs.

In another aspect, the present invention provides for a method of quantitating a type 2 immune response, comprising detecting a type 2 immune response in a subject in need thereof, wherein when an increased type 2 immune response is detected, the subject is treated according to any embodiment herein.

In certain embodiments, the methods described herein are used to treat IBD. In certain embodiments, IBD comprises a disease selected from the group consisting of ulcerative colitis (UC), Crohn's Disease, collagenous colitis, lymphocytic colitis, ischemic colitis, diversion colitis, Behcet's syndrome, infective colitis, indeterminate colitis, and other disorders characterized by inflammation of the mucosal layer of the large intestine or colon.

In another aspect, the present invention provides for a method of screening for one or more agents capable of modulating an ILC2 immune response comprising administering to a population of cells comprising ILC2s, mast cells, Th2 cells and/or fibroblasts one or more agents; and detecting expression, activity or function of one or more biological programs characterized by ILC Topic 2, myeloid cell Topic 1, T cell Topic 5 or stromal cell Topic 4, wherein ILC Topic 2 comprises one or more genes or polypeptides selected from the group consisting of Calca, Hs3st1, Areg, Il13, Il4, Ccl1, Hes1, Il17rb, Lgals7, Homer2, Il5, Gata3, Deptor, Ptpn13, Ly6a, Hba-a1, Kcnn4, Ccr4, Rxrg, Sub1, 1700061F12Rik, Cntnap2, AA467197, Ptgir, Il10, Nfkb1, Lmo4, Pparg, Plaur, Il9r, Serpine1, Scel, Bmp7, Neb, Sox8, Lpcat2, Samsn1, Alox5, Gpr65, Abhd17c, Gm20186, Gm973, Epas1, Ccr8, D430036J16Rik, Cd6, Stxbp6, 9230102O04Rik, Furin and Klf5, wherein myeloid cell Topic 1 comprises one or more genes or polypeptides selected from the group consisting of Cpa3, Cma1, Mcpt4, Tpsb2, Fcer1a, Hs3st1, Gata2, Cited4, Cyp11a1, Tph1, Furin, Rab27b, Slc45a3, Ccl1, Il13, Il1rl1, Itga2b, Cited2, Fam110c, Creb3l1, Rgs13, Tpsab1, Cyp26a1, Serpinb1a, Slc18a2, Gmpr, Rprm, Ero1l, Il4, Cd200r3, Glul, Kit, Lat, Alox5, Gchfr, mt-Atp6, Lat2, Prss34, Poln, Klk8, 4932438H23Rik, Slc6a13, Avil, Socs2, Smco4, Ier3, Lxn, Gpr171, Adk and Gata1, wherein T cell Topic 5 comprises one or more genes or polypeptides selected from the group consisting of: 1700061F12Rik, Il13, Scin, Lgmn, Hlf, Smco4, Npnt, Il17rb, Deptor, Gata3, Gm2a, Il6, Il17a, Ltb4r1, Fgl2, Areg, Fbxl21, AA467197, 1r11, Me1, Gm5544, Tmem159, Rasgrp4, 1700012B07Rik, 1700113H08Rik, St6galnac5, Il4, Chdh, Slco2b1, Ccr9, Epas1, Grp, Lztfl1, Gm10369, Kif19a, Tenm4, Serpinf1, Gnb2, Ubox5, Plcl1, Rab31, Ffar2, Slx1b, Asb2, Zfp85, Tmsb4x, Hdc, Pxdc1, Heatr1 and Lgals7, wherein stromal cell Topic 4 comprises one or more genes or polypeptides selected from the group consisting of Ccl21a, Cxcl13, Clu, Ccl19, Acta2, Mfge8, Apoe, Tagln, Cxcl1, Cilp, Ccl2, Il33, Cxcl12, Actg2, Serpina3n, Ccl7, Bst1, Serpina1a, Fmod, Grem1, Serpina1b, Slc36a2, Cnn1, Myh11, Art2b, Actc1, AI838599, Serpina1c, Cr2, Gxylt2, Crym, Dclk1, Serpina1d, Myl9, Parm1, Gm16685, Postn, Chrdl1, Colq, Csn2, Prss12, H2-M2, Trf, Sostdc1, Dsc3, Ctgf, Thbs4, Pcdh15, Rtn4r and A230065H16Rik.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—A single-cell expression atlas of intestinal immune cells. (A) Overview. The lamina propria (LP) and Peyer's patch (PP) regions of the small intestine were harvested from mice in homeostasis (“healthy”) or after induction of a type 2 inflammatory reaction to ovalbumin (OVA) (“inflamed”) (left). After enzymatic tissue dissociation, immune cells were captured by fluorescence-activated cell sorting (FACS) (middle) and analyzed by droplet-based 3′scRNA-seq (right). (B) Cell subsets in the intestinal immune cell atlas. scvis learned two-dimensional (2D) representation of 58,067 cell profiles (dots) from both PP and LP regions, shaded and numbered by cluster membership. Clustered are rank ordered by size (from the largest of 7194, cluster 1, to the smallest of 17, cluster 46). DZ, dark zone; LZ, light zone; DN, double negative; C1, cluster 1; C2, cluster 2. (C) Differentially expressed genes. For representative differentially expressed genes (rows) across clusters (columns), shown is the fraction of cells in the cluster that express a gene (dot size) and the z-score of the mean expression of that gene in the cluster (shading; z-score of average log²(TPM+1)). (D) Tissue distribution of non-T and non-B immune cells in homeostasis. Proportion (y axis) of each cell type in LP (triangle) and PP (black circle) regions. Points: independent experiments; Box and-whisker plots show the median, quartiles, and range. *p<4.8×10⁻³, **p<3.8×10⁻⁶, ***p<4.3×10⁻⁷, Wald test. (E) LTi cells are enriched in PP regions. Shown is flow cytometry analysis gated on CD45⁺Lin⁻ CD90.2⁺IL7R⁺RORγt⁺ cells. Left: Numbers adjacent to outlined areas indicate percent ILC3 (left) or LTi cells (right). Right: Summary of results. n=3 mice, mean and SEM; ***p<0.001, Fisher's exact test.

FIG. 2—Increased ILC2 proportions and changes in ILC2 programs are key features in OVA-induced type 2 inflammation. (A-D) Increased ILC2 and mast cell frequencies are prominent features in type 2 inflammation. (A) A 2D embedding as in FIG. 1B, where each cell profile (dot) is shaded by whether it was obtained in homeostatic or OVA-induced inflammatory conditions. (B) Distribution of cell type proportions (y axis) for each non-T and non-B cell subset (x axis) in homeostatic (left bar) or inflammatory (right bar) conditions. Points: independent experiments. Box and-whisker plots show the median, quartiles, and range. *p<1.3×10⁻², **p<4.5×10⁻⁴, ***p<6.1×10⁻⁸, Wald test. (C and D) Flow cytometry analysis of frequencies of mast cells (C, gated on CD45⁺Lin⁻) and ILC2s (D, gated on CD45⁺Lin⁻ CD90.2⁺IL7R⁺) in mice treated with PBS (black) or OVA (grey). Left: representative FACS plots; Right: summary of results. Points: individual mice; data are from two independent experiments. mean and SEM; **p<0.01, Student's t test. (E) ILC2s and mast cells show the most prominent changes in expression programs in type 2 inflammation. Number of genes significantly induced (black) or inhibited (grey) (|fold change|≥2, FDR<0.05, expressed at least in 25% of cells at the up-regulation side; shaded bar) by OVA-induced type 2 inflammation in each cell subset (column) from the LP or PP regions (label on left).

FIG. 3—Topic modeling of cell type-specific programs in response to intestinal type 2. inflammation predicts Calca as a top gene in ILC2s (A-E) Inflammation associated programs in specific cell subsets. Shown are some of the topics that have differential scores between steady state (PBS) and inflammatory conditions (OVA) for myeloid cells (A), ILCs (B), T cells (C, D) or stromal cells (E). Left: Bar plot shows the score (x axis) of top ranked genes for this topic (y axis). Top right: a portion of the 2D embedding in FIG. 1B, showing only cells from the noted subset cells, shaded by the topic's weight in the cell (top right). Bottom right: empirical cumulative density function (y axis) of topic weights (x axis) for cells from mice treated with PBS (black curve) or OVA (grey curve). p values: Mann-Whitney U test. (F) Increased number of IL-33⁺PDPN⁺ fibroblasts in inflammation. Left: Representative IF images of small intestines from mice at steady state (PBS) or inflammatory (OVA) conditions. Arrow, IL-33⁺PDPN⁺ fibroblasts; Scale bars, 50 Right: Quantified cell densities. Points: individual mice; data are from two independent experiments. mean and SEM, **p<0.01, Student's t test.

FIG. 4—α-CGRP suppresses IL-25-induced activation and expansion of intestinal KLRG1⁺ ILC2s in vitro. (A) Cells expressing Calca gene. 2D embedding as in FIG. 1B where cells (dots) are colored by relative expression of Calca (log²(TPM+1)). (B) α-CGRP exon specifically induced in inflammation. Expression level (y axis, Quantitative PCR normalized to Gapdh) of α-CGRP exon of Calca gene, Calcrl, and Ramp1 in intestinal KLRG1⁺ ILC2s isolated from control (black) or OVA-treated (grey) mice. Points: individual experiment; mean and SEM, *p<0.01, **p<0.01, Student's t test. (C and D) α-CGRP co-treatment abrogates most of the IL-25 induced response in ILC2s in vitro. (C) Expression (shaded bar, Z score) of genes (rows) significantly induced in ILC2s (Fold change≥2, FDR<0.05) by IL-25 compared to control, across different conditions (columns). (D) Box and whisker (min to max) plot of the average Z scores (y axis) of the IL-25-induced genes in (C). ***false discovery rate [FDR]<0.001, Student's t test. (E) α-CGRP suppresses IL-25-induced proliferation of ILC2s. Left: Distribution of number of cell divisions (y axis, % of maximum observed number) monitored by flow cytometry in KLRG1⁺ILC2s labeled with CellTrace Violet and stimulated with IL-25 or IL-25 plus α-CGRP for 60 hours. Right: Percent of dividing cells (y axis) in each treatment (x axis). Points: individual experiments. mean and SEM, *p<0.05, Student's t test.

FIG. 5—α-CGRP antagonizes expansion of intestinal KLRG1⁺ ILC2s in vivo. (A) Experimental design. Mice were intraperitoneally injected with vehicle, IL-25 or IL-25 and α-CGRP daily for two days. (B) α-CGRP co-treatment antagonizes CD3⁻KLRG1⁺ ILC2 expansion in vivo. Representative IF images of CD3⁻KLRG1⁺ ILC2 (arrows) in small intestines of mice treated as in A. Scale bars, 100 Data are representative of three mice in each condition. (C-E) α-CGRP antagonizes the expansion of ST2⁻KLRG1⁺ ILC2s specifically. (C) Flow cytometry analysis of ILC2s in mLNs cells gated on CD45⁺Lin⁻CD90.2⁺IL7R⁺ from mice treated as in (A). (D) Frequency (y axis) of ST2⁻KLRG1⁺ ILC2s in total CD45⁺ cells in mLNs of mice treated as in A. Points: individual mice. Data from three independent experiments. mean and SEM, *FDR<0.05, ***FDR<0.001, Student's t test. (E) Frequency (y axis) of ST2⁺ ILC2s in total CD45⁺ cells in mLNs of mice treated as in A. Point: individual mice. Data are from three independent experiments. mean and SEM, ns: FDR>0.05, *FDR<0.05, **FDR<0.01, Student's t test. (F and G) α-CGRP treatment antagonizes intestinal ILC2 expansion following OVA intraperitoneal sensitization experiment. (F) Experimental overview. (G) Percent of GATA3⁺ ILC2 (y axis) in total CD45⁺ cells in the small intestine of mice treated as in F. Point: individual mice. Data are from three independent experiments. mean and SEM, *p<0.05, Student's t test.

FIG. 6—α-CGRP is produced by ChAT⁺ enteric neurons in steady state and maintains KLRG1⁺ ILC2 homeostasis in vivo. (A and B) ChAT⁺ enteric neurons express α-CGRP. (A) t-SNE plot of 1105 enteric neuron profiles from Wnt1-Cre:R26Tomato mice (Zeisel et al., 2018), where each neuron (dot) is numbered by cluster assignment. (B) Distribution of expression levels (y axis, log₂(TPM+1)) of Nmu, Calca, Calcb and Calcrl gene in the enteric neurons in each cluster in A (x axis). (C) co-localization of ChAT and CGRP in the small intestines. Representative IF images of the small intestines of wild type mice. Scale bar, 50 Data are representative of three mice. (D) Expression program induced by α-CGRP in ILC2s in vitro. Relative expression (rowwise Z score of log₂(FPKM+1)) of genes (rows) significantly induced or suppressed by α-CGRP stimulation (|Fold change|≥2, FDR<0.05), across conditions (columns). Key genes are highlighted. (E) α-CGRP in ILC2s in vitro reprograms chromatin accessibility especially at loci of a cAMP response module. Left: Chromatin accessibility (log₂(#cuts+1), Methods) of loci (rows) with differential accessibility in ILC2s treated by α-CGRP vs. control in vitro. Right: Statistical significance (x axis, −log₁₀(q-value)) of molecular functions (y axis) whose associated genes are enriched in proximity to these differentially accessible ATAC-Seq peaks. Dashed line: qvalue=0.01. (F and G) Foskolin suppresses ILC2 proliferation but does not impact cell viability. (F) Left: Distribution of number of cell divisions (y axis, % of maximum observed number) monitored by flow cytometry in KLRG1⁺ ILC2s labeled with CellTrace Violet and stimulated with IL-25 and DMSO or forskolin for 40 hours. Right: Percent of divided cells (y axis) in each treatment (x axis). Points: individual experiments. mean and SEM, ***p<0.001, Student's t test. (G) Left: Cell viability monitored by flow cytometry in cells in F stained with 7AAD. Right: Percent of live (7AAD⁻) cells (y axis) in each treatment (x axis). Points: individual experiments. mean and SEM, NS: p>0.05, Student's t test. (H) Adenylate cyclase inhibitor (SQ22, 536) treatment partially rescues α-CGRP inhibition of ILC2 proliferation. Percent of divided cells (y axis) assayed as in F, in ILC2s treated in vivo with IL-25 and DMSO alone (black) or with α-CGRP (grey) or α-CGRP+Forskolin (dark grey). Points: individual experiments. mean and SEM, **p<0.01,*** p<0.001, Student's t test. (I and J) α-CGRP KO affects ILC2 expansion in vivo. (I) Left: Flow cytometry analysis of intestinal KLRG1⁺ cells among cells gated on CD45⁺Lin⁻ CD90.2⁺IL7R⁺ in α-CGRP WT and KO mice in homeostasis. Right: Percentage of KLRG1⁺ILC2s (y axis) in α-CGRP WT and KO mice. Points: individual mice. Data from three independent experiments. mean and SEM, **p<0.01, Student's t test. (J) Left: Representative IF images of tuft cells (arrows) in the small intestine of α-CGRP WT or KO mice. Scale bars, 50 Right: Density of tuft cells (y axis, number per mm²) in WT and KO. Points: individual mice. Data are from the independent experiments. mean and SEM, *p<0.05, Student's t test.

FIG. 7—A model of neuronal signals orchestrating ILC2 responses. Intestinal ILC2s sense different neuropeptides, including NMU, epinephrine (EPI) and CGRP from enteric neurons via expressing corresponding GPCRs. The amplifier NMUR1 signals through Gα_{q/11} and inhibits cAMP level, whereas the negative regulators CRLR and β₂AR are coupled with Gαₛ protein that results in cAMP accumulation. cAMP suppresses expansion of ILC2s but increases IL-5 expression. Thus, selective activation of different subgroups of a subunits of G proteins coupled with variable concentration of cAMP might be utilized by ILC2s to fine-turn the response to neuronal signaling.

FIG. 8—FACS, single-cell expression and IF staining analysis of intestinal immune cells, related to FIG. 1. (A) Experimental overview. (B) Depletion of common cell types by FACS. Shown are representative FACS plots for sorting lgD^{low} cells (left) or CD3⁻CD19⁻ cells (right) from PP and LP regions. CD45⁻ cells were not included in LP. (C) An LP and PP cell atlas. A 2D embedding as in FIG. 1B, but showing separately only the 36,797 cells from PP regions (left) or the 21,270 cells from LP (right). (D) Cluster quality measures. Top: Distribution of number of UMIs (y axis, log₁₀-transformed) in both LP and PP cells in each cluster from FIG. 1B (x axis). Bottom: distribution of doublet scores (y axis, Methods) of only PP cells in each cluster from FIG. 1B (x axis). (E) Clusters of specific cell types in the atlas. A 2D embedding as in FIG. 1B, but showing separately only the cells in each cell lineage or group as labeled. (F) Differentially expressed genes between ILC3 and LTi. Fraction of cells in the cluster that express a gene (dot size) and the z-score of the mean expression of that gene in those cells that express it in the cluster (shading; z-score of average log²(TPM+1)). (G) Tissue distribution of B (left) and T (right) cells between LP and PP in homeostasis. Proportion (y axis) of each cell type (x axis) in LP (triangle) and PP (black circle) regions. Points: independent experiments; Box-and-whisker plots show the median, quartiles, and range. *p<6×10⁻⁰⁵, **p<4×10⁻¹⁰, ***p<2.5×10⁻¹⁵³ Wald test. (H) Differential distribution of plasma cells and GC B cells. Representative IF image of the small intestines of wild type mice. Dashed line: PP region; Small dashed region: GC area; arrow: CD138⁺ plasma cell; scale bar, 100 μm. Data are representative of three mice.

FIG. 9—Cell proportions in T and B cells, and gene expression in ILC2s, mast cells, macrophages and plasma cells in OVA-induced type 2 inflammation, related to FIG. 2. (A) Distribution of cell type proportions (y axis) in B cells (left) and T cells in homeostatic (black) or inflammatory (grey) conditions. Points: independent experiments. Box-and-whisker plots show the median, quartiles, and range. (B) Differentially expressed genes in ILC2s, mast cells, macrophages and plasma cells. Shown are genes repressed (left of zero) or induced (right of zero) (|fold change|≥2, FDR<0.05, likelihood ratio test, frequency of expressing cells ≥25% at the up-regulation side) by the inflammation in each labeled cell type. Dots: genes, with key ones labeled. (C) Distribution of expression levels (y axis, log²(TPM+1)) of Igh genes (labeled) in plasma cells in homeostatic (left) or inflammatory (right) conditions. *p<2.8×10⁻⁵, **p<2.9×10⁻¹³, ***p<1.5×10⁻¹⁰⁷, likelihood ratio test.

FIG. 10—Topic modeling on T cells, B cells, DCs, ILCs, myeloid cells and stromal cells, and co-expression of CCL21 and IL-33 in PDPN⁺ stromal cells in the small intestine, related to FIG. 3. (A-F) Biological programs in each cell subset revealed by topic modeling. Shown are topics in T cells (A), B cells (B), ILCs (C), myeloid cells (D), DCs (E) and stromal cells (F). Top (A-D) and left (E and F): a portion of the 2D embedding in FIG. 1B, showing only cells from the noted subset cells, shaded by the topic's weight in the cell. Bottom (A-D) and right (E and F): empirical cumulative density function (y axis) of topic weights (x axis) for cells from mice treated with PBS (black curve) or OVA (grey curve). p values: Mann-Whitney U test. (G) co-localization of CCL21 and IL-33 in subsets of PDPN⁺ stromal cells (arrow) in the small intestines. Representative IF images of the small intestines of wild type mice. Scale bars, 50 μm. Data are representative of three mice.

FIG. 11—Expression of Calca, Calcrl, Ramp1 and Il5 genes in intestinal KLRG1⁺ ILC2s in homeostatic and inflammatory conditions, related to FIG. 4. (A) Distribution of expression levels (y axis, log₂(TPM+1)) of Calca (top), Calcrl (middle), Ramp1 (bottom) in each cell types (x axis) in mice treated with PBS (black) or OVA (grey). ILC2s are highlighted. *P<0.05, **P<2.3×10⁻⁰⁶, likelihood ratio test. (B) Expression level (y axis, Quantitative PCR normalized to Gapdh) of CT exon of Calca gene in intestinal KLRG1⁺ ILC2s isolated from PBS or OVA-treated mice. Points: individual experiment; mean and SEM, NS: p>0.05, Student's t test. (C) α-CGRP promotes Il5 expression in intestinal ILC2s. Shown are Z scores of Il5 gene in FIG. 4C. Points: individual sample; mean and SEM, *FDR<0.05, **FDR<0.01, ***FDR<0.001, Student's t test.

FIG. 12—α-CGRP suppress expansion of intestinal KLRG1⁺ ILC2s in two in vivo models, related to FIG. 5. (A) α-CGRP co-treatment antagonizes CD3⁻KLRG1⁺ ILC2 expansion in vivo. Representative IF images of CD3⁻KLRG1⁺ ILC2 (arrows) in small intestines of mice treated as in FIG. 5A. Scale bars, 200 Data are representative of three mice in each condition. (B) α-CGRP suppresses the expansion of ST2⁻KLRG1⁺ ILC2s in mLNs. Quantification of frequency (y axis) of ST2⁻KLRG1⁺ ILC2s in FIG. 5C. Points: individual mice. Data from three independent experiments. mean and SEM, *FDR<0.05, ***FDR<0.001, Student's t test. (C, D) α-CGRP treatment antagonizes intestinal ILC2 expansion in OVA-induced inflammation model. (C) Flow cytometry analysis of ILC2s (gated on CD45⁺Lin⁻ CD90.2⁺IL7R⁺) in the small intestines in mice treated as in FIG. 5F. (D) Percent of GATA3⁺ ILC2 (y axis) in all ILCs in the small intestine of mice in (C). Points: individual mice. Data from three independent experiments. mean and SEM, **p<0.01, Student's t test.

FIG. 13—scRNA-seq analysis of intestinal epithelium and enteric neurons, and ATAC-seq analysis of intestinal ILC2s, related to FIG. 6. (A) Undetectable expression of Calca and Calcrl in epithelium of wild type mice (Haber et al, 2017). Distribution of expression levels (y axis, log₂(TPM+1)) of Calca (top), Ramp1 (middle), Calcrl (bottom) in each epithelial cell type as labeled. (B) Marker genes of each enteric neuron clusters. Heatmap showing relative expression (Z score of log₂(TPM+1)) of top 5 marker genes (row) of each cluster (column) in FIG. 6A. (C) Distribution of expression levels (y axis, log₂(TPM+1)) of Chat, Gal, Il13ra1, Il4ra and Tslp gene in each cluster (x axis) in FIG. 6A. (D) Interaction between ChAT-expressing neuron fibers and CD3⁻KLRG1⁺ ILC2s (arrow) in the small intestines. Representative IF images of the small intestines of wild type mice. Scale bar, 25 Data are representative of three mice.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboraotry Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to International Application Nos. PCT/US2018/024082, published as WO 2018/175924A1 on Sep. 27, 2018, and PCT/US2019/030911, published as WO2019/213660A2 on Nov. 7, 2019. Reference is also made to Xu et al., Transcriptional Atlas of Intestinal Immune Cells Reveals that Neuropeptide α-CGRP Modulates Group 2 Innate Lymphoid Cell Responses. Immunity. 2019 Oct. 15; 51(4):696-708.e9.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide methods and compositions for modulating an innate immune response, in particular an innate lymphoid cell class 2 innate immune response by modulating activity of CGRP signaling. Embodiments disclosed herein also provide for methods of monitoring an innate lymphoid cell class 2 innate immune response in response to disease or treatment.

It is an objective of the present invention to identify molecular cues that modulate ILC2 responses to alarmins (e.g., for therapeutic applications). It is an objective of the present invention to modulate ILC2 immune responses and cell states using CGRP either alone or in combination with other treatments. It is another objective to modulate ILC2 immune responses using CGRP in combination with agents currently in use for the modulation of immune responses or associated with regulation of immune responses.

Signaling abnormalities in immune responses in the small intestine can trigger chronic type 2 inflammation, involving interaction of multiple immune cell types. Here, Applicants combine scRNA-seq with physiological and genetic perturbations to dissect the cellular circuit of type 2 intestinal inflammation. To systematically characterize this response, Applicants analyzed 58,067 immune cells from the mouse small intestine by single-cell RNA-seq (scRNA-seq) at steady state and after induction of a type 2 inflammatory reaction to ovalbumin (OVA). Specifically, to uncover key responding cellular components, Applicants profiled individual immune cells in the lamina propria (LP) and Peyer's patches (PPs), regions of the small intestine enriched for immune cells, in homeostasis and in an intestinal type 2 inflammatory model. Analysis revealed broad shifts in both cell type composition and cell programs in response to the inflammation, especially in ILC2s. Among the key transcripts associated with an inflammation-induced program in intestinal KLRG1⁺ ILC2s was exon 5 of Calca, which encodes the alpha-calcitonin gene-related peptide (α-CGRP). Specifically, computational analysis showed that both cell compositions and cell programs shifted in response to inflammation, in particular the prominent induction of α-CGRP transcription in ILC2s. α-CGRP antagonized IL-25-induced activation of intestinal KLRG1⁺ILC2s and reduced ILC2 frequency in an OVA reaction model. In inflammatory conditions, α-CGRP suppressed activation of ILC2s, but induced IL-5 expression. In vivo stimulation with α-CGRP alone induced the expression of a cyclic AMP (cAMP) response gene module and suppressed cell proliferation. In homeostasis in vivo, α-CGRP was predominantly expressed by two subsets of ChAT⁺ enteric neurons, and genetic perturbation of α-CGRP increased the proportion of intestinal KLRG1⁺ ILC2s and the number of Tuft cells. Embodiments disclosed herein provide methods for targeting α-CGRP-mediated neuronal signaling for suppressing ILC2 expansion and maintaining homeostasis of the type 2 immune machinery.

The discovery presented herein highlights the importance of neuro-immune crosstalk in allergic inflammatory responses at mucosal surfaces. Moreover, Applicants have discovered novel regulatory mechanisms for modulating the balance between tissue protective ILCs and tissue inflammatory cells. In certain embodiments, the methods and compositions described herein may be used to shift the balance of ILC2 responses in order to treat inflammatory allergic diseases and cancer.

### Expression Signatures

In certain example embodiments, the therapeutic, diagnostic, and screening methods disclosed herein target, detect, or otherwise make use of one or more biomarkers of an expression signature. As used herein, the term “biomarker” can refer to a gene, an mRNA, cDNA, an antisense transcript, a miRNA, a polypeptide, a protein, a protein fragment, or any other nucleic acid sequence or polypeptide sequence that indicates either gene expression levels or protein production levels. Accordingly, it should be understood that reference to a “signature” in the context of those embodiments may encompass any biomarker or biomarkers whose expression profile or whose occurrence is associated with a specific cell type, subtype, or cell state of a specific cell type or subtype within a population of cells (e.g., inflammatory or homeostatic ILC2 cells) or a specific biological program. As used herein the term “module” or “biological program” can be used interchangeably with “expression program” and refers to a set of biomarkers that share a role in a biological function (e.g., an activation program, cell differentiation program, proliferation program). Biological programs can include a pattern of biomarker expression that result in a corresponding physiological event or phenotypic trait. Biological programs can include up to several hundred biomarkers that are expressed in a spatially and temporally controlled fashion. Expression of individual biomarkers can be shared between biological programs. Expression of individual biomarkers can be shared among different single cell types; however, expression of a biological program may be cell type specific or temporally specific (e.g., the biological program is expressed in a cell type at a specific time). Expression of a biological program may be regulated by a master switch, such as a nuclear receptor or transcription factor. As used herein, the term “topic” refers to a biological program as determined by topic modeling. Topics are described further herein. The biological program (topic) can be modeled as a distribution over expressed biomarkers.

In certain embodiments, the expression of the signatures disclosed herein (e.g., inflammatory, homeostatic or CGRP signature) is dependent on epigenetic modification of the biomarkers or regulatory elements associated with the signatures (e.g., chromatin modifications or chromatin accessibility). Thus, in certain embodiments, use of signature biomarkers includes epigenetic modifications of the biomarkers that may be detected or modulated. As used herein, the terms “signature”, “expression profile”, or “expression program” may be used interchangeably (e.g., expression of genes, expression of gene products or polypeptides). It is to be understood that also when referring to proteins (e.g. differentially expressed proteins), such may fall within the definition of “gene” signature. Levels of expression or activity may be compared between different cells in order to characterize or identify, for instance, signatures specific for cell (sub)populations. Increased or decreased expression or activity or prevalence of signature biomarkers may be compared between different cells in order to characterize or identify for instance specific cell (sub)populations. The detection of a signature in single cells may be used to identify and quantitate for instance specific cell (sub)populations. A signature may include a biomarker whose expression or occurrence is specific to a cell (sub)population, such that expression or occurrence is exclusive to the cell (sub)population. An expression signature as used herein, may thus refer to any set of up- and/or down-regulated biomarkers that are representative of a cell type or subtype. An expression signature as used herein, may also refer to any set of up- and/or down-regulated biomarkers between different cells or cell (sub)populations derived from a gene-expression profile. For example, an expression signature may comprise a list of biomarkers differentially expressed in a distinction of interest.

The signature according to certain embodiments of the present invention may comprise or consist of one or more biomarkers, such as for instance 1, 2, 3, 4, 5, 6, 7, 8, 9, 10 or more. In certain embodiments, the signature may comprise or consist of two or more biomarkers, such as for instance 2, 3, 4, 5, 6, 7, 8, 9, 10 or more. In certain embodiments, the signature may comprise or consist of three or more biomarkers, such as for instance 3, 4, 5, 6, 7, 8, 9, 10 or more. In certain embodiments, the signature may comprise or consist of four or more biomarkers, such as for instance 4, 5, 6, 7, 8, 9, 10 or more. In certain embodiments, the signature may comprise or consist of five or more biomarkers, such as for instance 5, 6, 7, 8, 9, 10 or more. In certain embodiments, the signature may comprise or consist of six or more biomarkers for instance 6, 7, 8, 9, 10 or more. In certain embodiments, the signature may comprise or consist of seven or more biomarkers, such as for instance 7, 8, 9, 10 or more. In certain embodiments, the signature may comprise or consist of eight or more biomarkers, such as for instance 8, 9, 10 or more. In certain embodiments, the signature may comprise or consist of nine or more biomarkers, such as for instance 9, 10 or more. In certain embodiments, the signature may comprise or consist of ten or more biomarkers, such as for instance 10, 11, 12, 13, 14, 15, or more. It is to be understood that a signature according to the invention may for instance also include different types of biomarkers combined (e.g. genes and proteins).

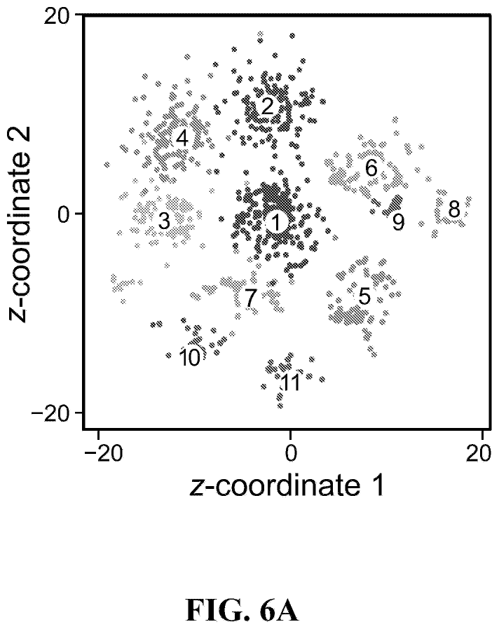

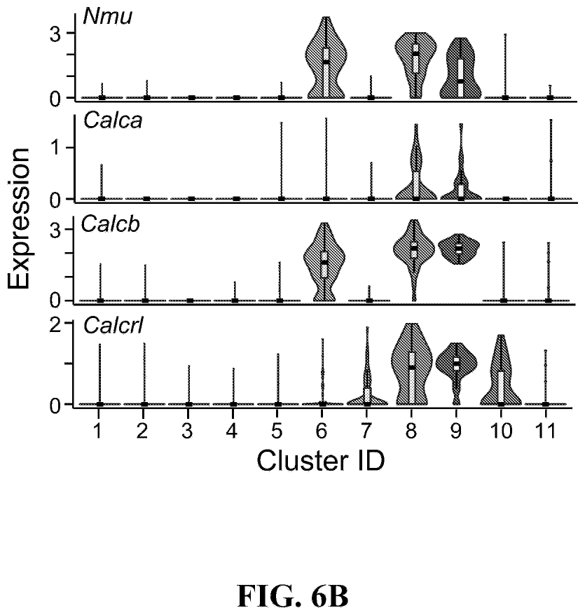

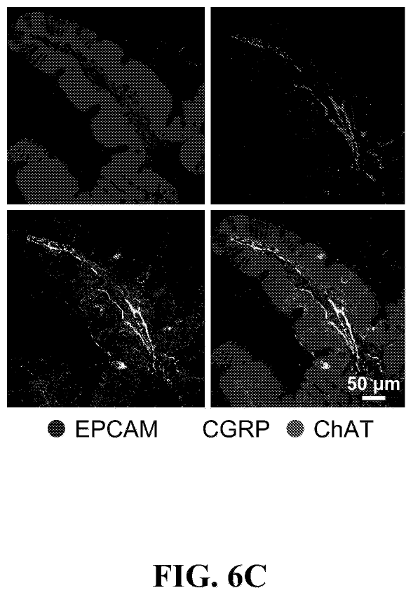

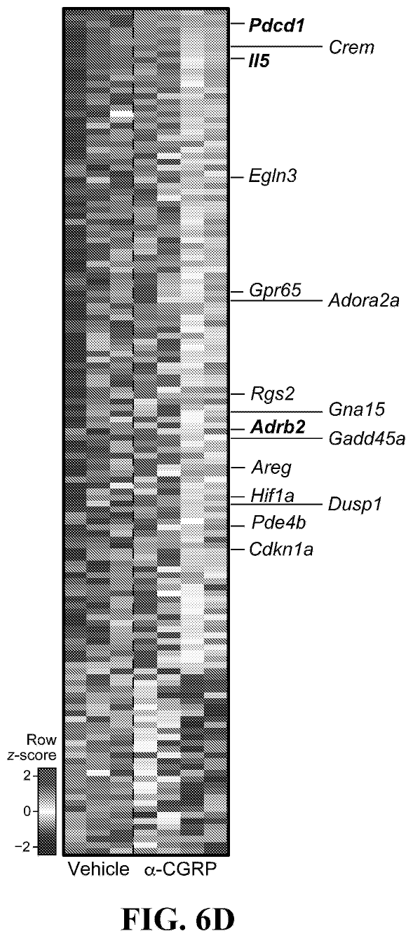

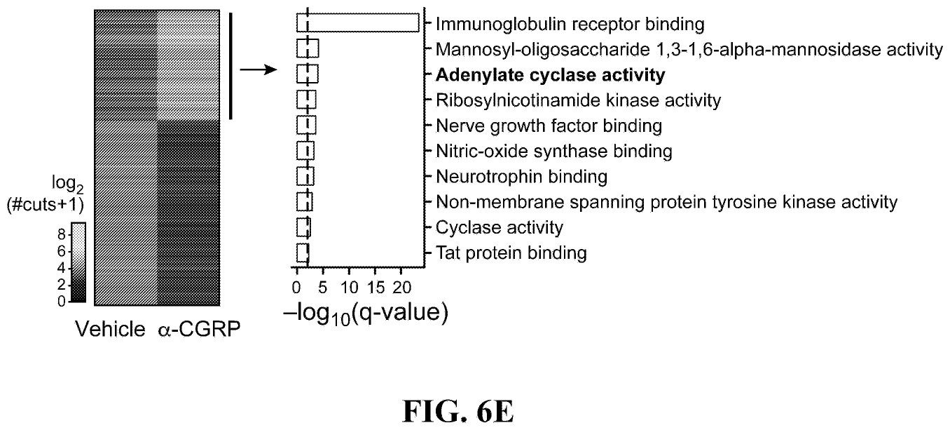

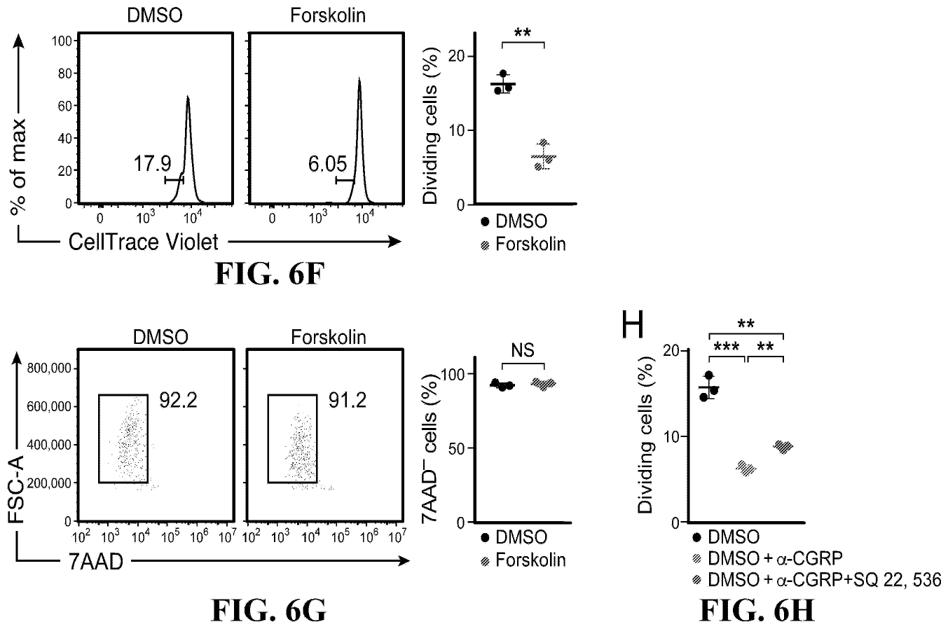

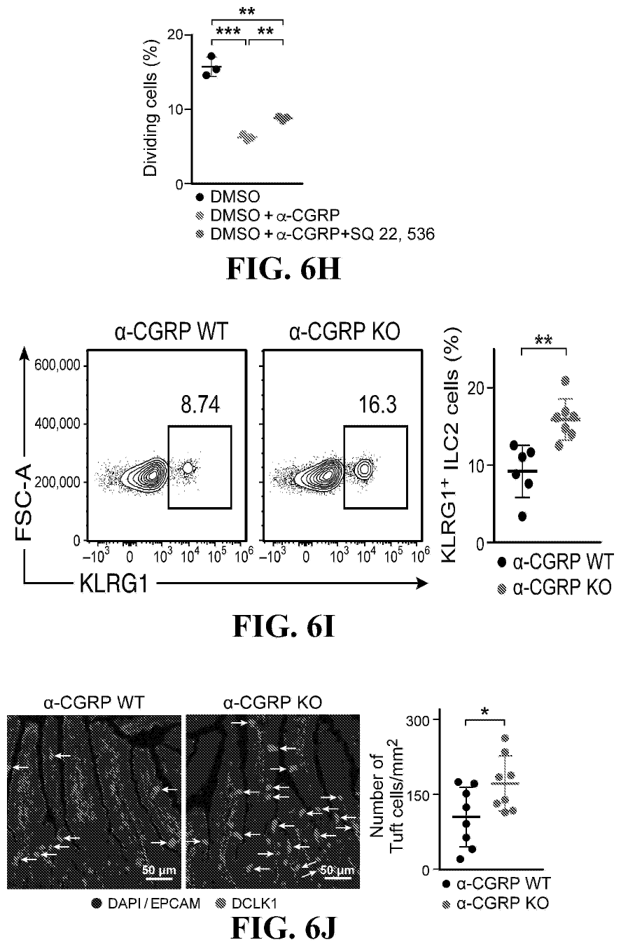

In certain embodiments, a signature is characterized as being specific for a particular cell or cell (sub)population if it is upregulated or only present, detected or detectable in that particular cell or cell (sub)population, or alternatively is downregulated or only absent, or undetectable in that particular cell or cell (sub)population. In this context, a signature consists of one or more differentially expressed genes/proteins or differential epigenetic elements when comparing different cells or cell (sub)populations, including comparing different immune cells or immune cell (sub)populations (e.g., ILC2 cells), as well as comparing immune cells or immune cell (sub)populations with other immune cells or immune cell (sub)populations. It is to be understood that “differentially expressed” biomarkers include biomarkers which are up- or down-regulated as well as biomarkers which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art. Differential expression of biomarkers may also be determined by comparing expression of biomarkers in a population of cells or in a single cell. In certain embodiments, expression of one or more biomarkers is mutually exclusive in cells having a different cell state or subtype (e.g., two genes are not expressed at the same time). In certain embodiments, a specific signature may have one or more biomarkers upregulated or downregulated as compared to other biomarkers in the signature within a single cell (see, e.g., FIG. 6D). Thus a cell type or subtype can be determined by determining the pattern of expression in a single cell.

As discussed herein, differentially expressed biomarkers may be differentially expressed on a single cell level, or may be differentially expressed on a cell population level. Preferably, the differentially expressed biomarkers as discussed herein, such as constituting the expression signatures as discussed herein, when as to the cell population level, refer to biomarkers that are differentially expressed in all or substantially all cells of the population (such as at least 80%, preferably at least 90%, such as at least 95% of the individual cells). This allows one to define a particular subpopulation of cells. As referred to herein, a “subpopulation” of cells preferably refers to a particular subset of cells of a particular cell type (e.g., ILC2) which can be distinguished or are uniquely identifiable and set apart from other cells of this cell type. The cell subpopulation may be phenotypically characterized, and is preferably characterized by the signature as discussed herein. A cell (sub)population as referred to herein may constitute of a (sub)population of cells of a particular cell type characterized by a specific cell state.

When referring to induction, or alternatively suppression of a particular signature, preferable is meant induction or alternatively suppression (or upregulation or downregulation) of at least one biomarker of the signature, such as for instance at least two, at least three, at least four, at least five, at least six, or all biomarkers of the signature.

Example gene signatures and topics are further described below.

### IL-25 Inflammatory ILC2 Gene Signature

In certain embodiments, an IL-25 inflammatory ILC2 gene signature (e.g., IL-25 induced genes; or signature of differentially expressed genes between ILC2s treated with IL-25 and IL-25+CGRP; or IL-25 induced genes that can be modulated by CGRP) comprises one or more biomarkers selected from Table A.

TABLE A

| | | |
|---|---|---|
| I15 | Furin | Gem |
| Nr4a1 | Ptgs2 | Il9 |
| Irf4 | Nfkbiz | Tph1 |
| Ccr4 | Thbd | Gadd45g |
| Egr2 | Ntn1 | Prelp |
| Il6 | Flt4 | Pecam1 |
| Myc | Fxyd6 | Pcl13 |
| Timp3 | Csf2 | Reln |
| Pim2 | Gpr97 | Aqp1 |
| Cntf | Mmrn1 | Ptger2 |
| Mras | Prss23 | Emcn |
| Cldn5 | Adam8 | Lyve1 |
| Il13 | Sdpr | Gstm1 |
| Lcn2 | Gm1987 | |

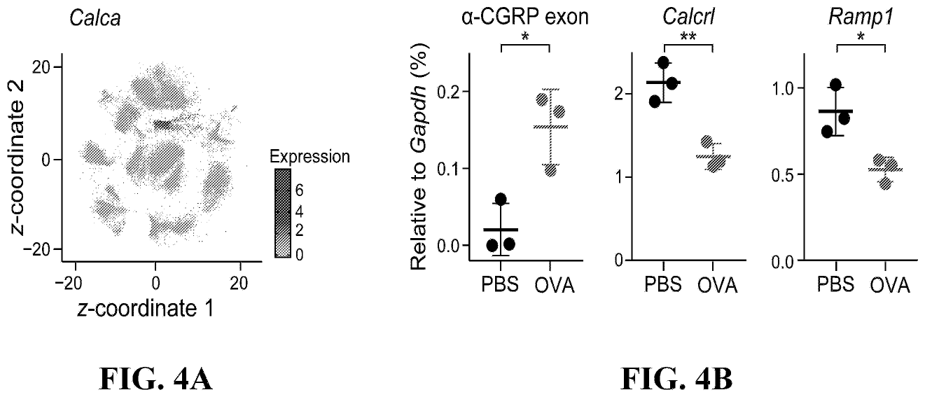

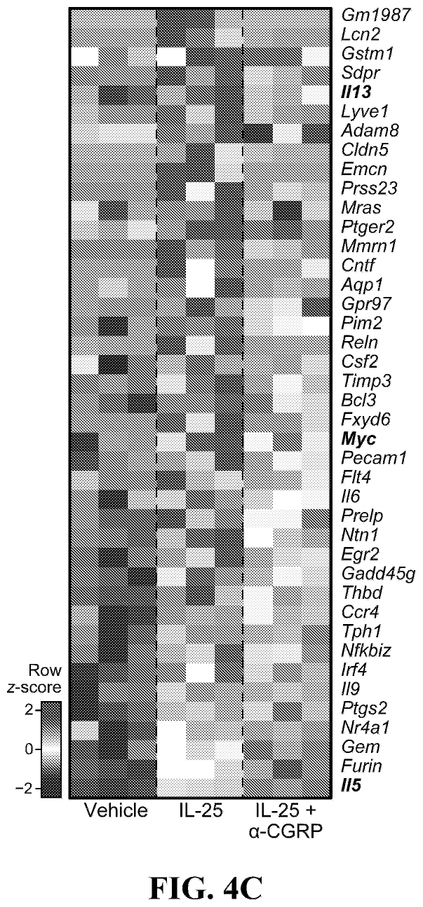

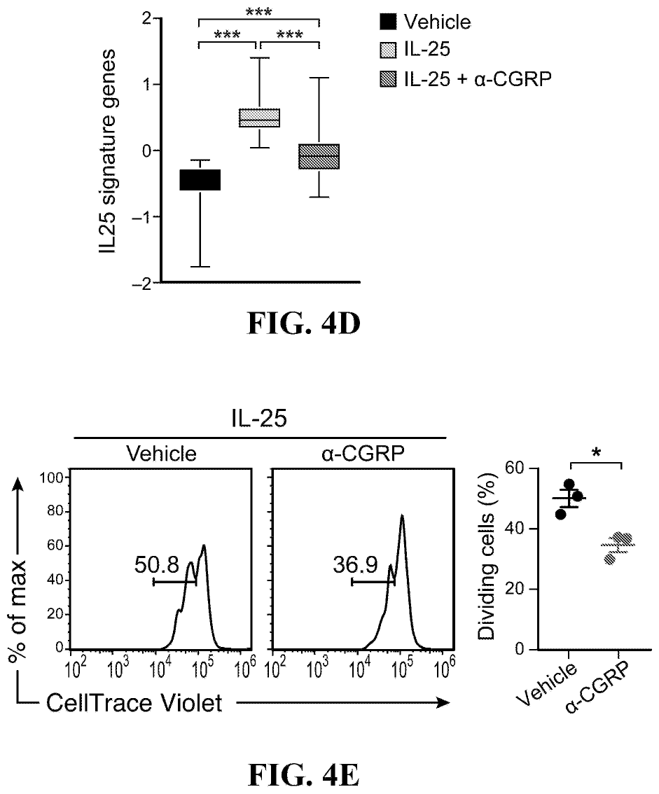

In one example embodiment, the IL-25 inflammatory ICL2 signature consists of the biomarkers Il5, Furin, Gem, Nr4a1, Ptgs2, Il9, Irf4, Nfkbiz, Tph1, Ccr4, Thbd, Gadd45g, Egr2, Ntn1, Prelp, Il6, Flt4, Pecam1, Myc, Fxyd6, Bcl3, Timp3, Csf2, Reln, Pim2, Gpr97, Aqp1, Cntf, Mmrn1, Ptger2, Mras, Prss23, Emcn, Cldn5, Adam8, Lyve1, Il13, Sdpr, Gstm1, Lcn2 and Gm1987 (see, FIG. 4C). In certain embodiments, IL-25 induces an inflammatory gene signature and this signature can be modulated (e.g., reversed or partially reversed) by treatment with CGRP.

In one example embodiment the IL-25 Inflammatory ILC2 signature comprises Il5 and at least N additional biomarker from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Furin and at least one of N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Gem and at least one of N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Nr4a1 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Ptgs2 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises 119 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Irf4 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Nfkbiz and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Tph1 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Ccr4 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Thbd and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Gadd45g and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Egr2 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Ntn1 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Prelp and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Il6 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Flt4 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Pecam1 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Myc and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Fxyd6 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Pcl13 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Timp3 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Csf2 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Reln and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Pim2 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Gpr97 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Aqp1 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Cntf and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Mmrn1 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Ptger2 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Mras and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Prss23 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Emcn and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Cldn5 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Adam8 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Lyve1 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Il13 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Sdpr and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Gstm1 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Lcn2 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In another example embodiment the IL-25 Inflammatory ILC2 signature comprises Gm1987 and at least N additional biomarkers from Table A, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

### ILC2 Signature (Topic 2)

In certain embodiments, an ILC2 expression signature (Topic 2) comprises one or more biomarkers selected from Table B.

TABLE B

| | | |
|---|---|---|
| Calca | Hs3st1 | Areg |
| Il13 | Il4 | Ccl1 |
| Hes1 | Il17rb | Lgals7 |
| Homer2 | Il5 | Gata3 |
| Deptor | Ptpn13 | Ly6a |
| Hba-a1 | Kcnn4 | Ccr4 |
| Rxrg | Sub1 | 1700061F12Rik |
| Cntnap2 | AA467197 | Ptgir |
| Il10 | Nfkb1 | Lmo4 |
| Pparg | Plaur | Il9r |
| Serpine1 | Scel | Bmp7 |
| Neb | Sox8 | Lpcat2 |
| Samsn1 | Alox5 | Gpr65 |
| Abhd17c | Gm20186 | Gm973 |
| Epas1 | Ccr8 | D430036J16Rik |
| Cd6 | Stxbp6 | 9230102O04Rik |
| Furin | Klf5. | |

In one example embodiment, the ILC2 signature consists of Calca, Hs3st1, Areg, Il13, Il4, Ccl1, Hes1, Il17rb, Lgals7, Homer2, Il5, Gata3, Deptor, Ptpn13, Ly6a, Hba-a1, Kcnn4, Ccr4, Rxrg, Sub1, 1700061F12Rik, Cntnap2, AA467197, Ptgir, Il10, Nfkb1, Lmo4, Pparg, Plaur, Il9r, Serpine1, Scel, Bmp7, Neb, Sox8, Lpcat2, Samsn1, Alox5, Gpr65, Abhd17c, Gm20186, Gm973, Epas1, Ccr8, D430036J16Rik, Cd6, Stxbp6, 9230102O04Rik.

In another example embodiment, the ILC2 signature comprises one gene from Table B and at least N additional biomarkers selected from Table B (e.g., Calca and one or more additional genes from Table B), wherein N is equal to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

### Myeloid Cell Signature (Topic 1)

In certain embodiments, myeloid cell signature (Topic 1) comprises one or more genes or polypeptides selected from the group consisting of: Cpa3, Cma1, Mcpt4, Tpsb2, Fcer1a, Hs3st1, Gata2, Cited4, Cyp11a1, Tph1, Furin, Rab27b, Slc45a3, Ccl1, Il13, Il1rl1, Itga2b, Cited2, Fam110c, Creb3l1, Rgs13, Tpsab1, Cyp26a1, Serpinb1a, Slc18a2, Gmpr, Rprm, Ero1l, Il4, Cd200r3, Glul, Kit, Lat, Alox5, Gchfr, mt-Atp6, Lat2, Prss34 Poln, Klk8, 4932438H23Rik, Slc6a13, Avil, Socs2, Smco4, Ier3, Lxn, Gpr171, Adk and Gata1.

In another example embodiment, the myeloid cell signature comprises one gene from topic 1 and at least N additional biomarkers selected from topic 1 (e.g., Cpa3 and one or more additional genes), wherein N is equal to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

### T Cell Signature (Topic 5)

In one example embodiment, T cell signature (Topic 5) comprises one or more genes or polypeptides selected from the group consisting of: 1700061F12Rik, Il13, Scin, Lgmn, Hlf, Smco4, Npnt, Il17rb, Deptor, Gata3, Gm2a, Il6, Il17a, Ltb4r1, Fgl2, Areg, Fbxl21, AA467197, Il1rl1, Me1, Gm5544, Tmem159, Rasgrp4, 1700012B07Rik, 1700113H08Rik, St6galnac5, Il4, Chdh, Slco2b1, Ccr9, Epas1, Grp, Lztfl1, Gm10369, Kif19a, Tenm4, Serpinf1, Gnb2, Ubox5, Plcl1, Rab31, Ffar2, Slx1b, Asb2, Zfp85, Tmsb4x, Hdc, Pxdc1, Heatr1 and Lgals7.

In another example embodiment, the T cell signature comprises one gene from topic 5 and at least N additional biomarkers selected from topic 5 (e.g., 1700061F12Rik and one or more additional genes), wherein N is equal to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

### Stromal Cell Signature (Topic 4)

In one example embodiment, a stromal cell signature (Topic 4) comprises one or more genes or polypeptides selected from the group consisting of: Ccl21a, Cxcl13, Clu, Ccl19, Acta2, Mfge8, Apoe, Tagln, Cxcl1, Cilp, Ccl2, Il33, Cxcl12, Actg2, Serpina3n, Ccl7, Bst1, Serpina1a, Fmod, Grem1, Serpina1b, Slc36a2, Cnn1, Myh11, Art2b, Actc1, AI838599, Serpina1c, Cr2, Gxylt2, Crym, Dclk1, Serpina1d, Myl9, Parm1, Gm16685, Postn, Chrdl1, Colq, Csn2, Prss12, H2-M2, Trf, Sostdc1, Dsc3, Ctgf, Thbs4, Pcdh15, Rtn4r and A230065H16Rik.

In another example embodiment, the stromal cell signature comprises one gene from topic 4 and at least N additional biomarkers selected from topic 4 (e.g., Ccl21a and one or more additional genes), wherein N is equal to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

### CGRP Signature

In certain embodiments, treatment of ILC2s with CGRP alone provides for a CGRP gene signature comprising one or more genes selected from the group consisting of: Gpr65, Pdcd1, Crem, Egln3, Adora2a, Rgs2, Gna15, Adrb2, Gadd45a, Areg, Hif1a, Dusp1, Pde4b, Cdkn1a, Akap12 and Il5 (see, FIG. 6D showing all genes differentially expressed upon CGRP treatment). In certain embodiments, this signature can be used to monitor an immune response or monitor a response to a treatment (e.g., CGRP). In certain embodiments, a shift to higher expression of the signature indicates that the treatment is reducing an inflammatory response or maintaining homeostasis.

In another example embodiment the CGRP signature comprises Gpr65 and at least N additional biomarkers from FIG. 6D, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or up to 150 genes shown in FIG. 6D.

In another example embodiment the CGRP signature comprises Pdcd1 and at least N additional biomarkers from FIG. 6D, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or up to 150 genes shown in FIG. 6D.

In another example embodiment the CGRP signature comprises Crem and at least N additional biomarkers from FIG. 6D, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or up to 150 genes shown in FIG. 6D.

In another example embodiment the CGRP signature comprises Egln3 and at least N additional biomarkers from FIG. 6D, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or up to 150 genes shown in FIG. 6D.

In another example embodiment the CGRP signature comprises Adora2a and at least N additional biomarkers from FIG. 6D, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or up to 150 genes shown in FIG. 6D.

In another example embodiment the CGRP signature comprises Rgs2 and at least N additional biomarkers from FIG. 6D, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or up to 150 genes shown in FIG. 6D.

In another example embodiment the CGRP signature comprises Gna15 and at least N additional biomarkers from FIG. 6D, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or up to 150 genes shown in FIG. 6D.

In another example embodiment the CGRP signature comprises Adrb2 and at least N additional biomarkers from FIG. 6D, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or up to 150 genes shown in FIG. 6D.

In another example embodiment the CGRP signature comprises Gadd45a and at least N additional biomarkers from FIG. 6D, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or up to 150 genes shown in FIG. 6D.

In another example embodiment the CGRP signature comprises Areg and at least N additional biomarkers from FIG. 6D, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or up to 150 genes shown in FIG. 6D.

In another example embodiment the CGRP signature comprises Hif1a and at least N additional biomarkers from FIG. 6D, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or up to 150 genes shown in FIG. 6D.

In another example embodiment the CGRP signature comprises Dusp1 and at least N additional biomarkers from FIG. 6D, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or up to 150 genes shown in FIG. 6D.

In another example embodiment the CGRP signature comprises Pde4b and at least N additional biomarkers from FIG. 6D, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or up to 150 genes shown in FIG. 6D.

In another example embodiment the CGRP signature comprises Cdkn1a and at least N additional biomarkers from FIG. 6D, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or up to 150 genes shown in FIG. 6D.

In another example embodiment the CGRP signature comprises Akap12 and at least N additional biomarkers from FIG. 6D, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or up to 150 genes shown in FIG. 6D.

In another example embodiment the CGRP signature comprises Il5 and at least N additional biomarkers from FIG. 6D, wherein N equals 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or up to 150 genes shown in FIG. 6D.

### Methods of Maintaining or Inducing Homeostasis of Intestinal ILC2 Cells

The following section provides multiple example embodiments for maintaining or inducing homeostasis of intestinal ILC2 cells. The methods may be administered to subjects at risk for having aberrant activation and or expansion of intestinal ILC2 cells. Thus, the embodiments may be used to prevent and/or treat diseases and disorders characterized by aberrant activation or expansion of intestinal ILC 2 cells.

### Modulations of CGRP

In one aspect, methods of maintaining or inducing homeostasis of intestinal ILC2 cells may comprise administering a CGRP, or functional domain thereof, to a subject in need thereof. In certain example embodiments, a subject in need thereof may be a subject at risk for or having aberrant activation and expansion of intestinal ICL2 cells. Examples of diseases or disorders characterized by aberrant activation and expansion of intestinal ILC2 cells include, but are not limited to allergies (e.g., food allergies). As used herein “maintaining” means that if ILC2s are at homeostasis they are maintained in that current state and do not become inflammatory. As used herein “inducing homeostasis” means increasing the amount of homeostatic ILC2s or switching inflammatory ILC2s to homeostatic ILC2s.

The CGRP protein (also known as: Calcitonin Related Polypeptide Alpha, Calcitonin, Calcitonin Gene-Related Peptide 1, Calcitonin Gene-Related Peptide I, Alpha-Type CGRP, Calcitonin 1, CGRP-I, CALC1, Calcitonin/Calcitonin-Related Polypeptide, Alpha, Katacalcin, CGRP1, CGRP, PCT, CT and KC) (HUGO Gene Nomenclature Committee ID NO. HGNC:10489) may be any α-CGRP or β-CGRP, their functional variants, functional fragments or any mammalian orthologues thereof. In certain example embodiments, CGRP also includes peptides having undergone post-translational modifications, such as peptides having covalent attachment of glycosyl groups, acetyl groups, phosphate groups, lipid groups, and the like.

The human peptide α-CGRP (UniProtKB/Swiss-Prot ref: P06881.3) is encoded by the human gene CALCA (NCBI ref: NG_015960.1, NP_001029125.1) and has the sequence: Ala-Cys-Asp-Thr-Ala-Thr-Cys-Val-Thr-His-Arg-Leu-Ala-Gly-Leu-Leu-Ser-Arg-Ser-Gly-Gly-Val-Val-Lys-Asn-Asn-Phe-Val-Pro-Thr-Asn-Val-Gly-Ser- Lys-Ala-Phe-NH2 (SEQ ID NO: 1). In certain example embodiments, the CGRP to be administered is human α-CGRP. In certain example embodiments, the human α-CGRP to be administered is SEQ ID NO: 1 or a functional variant or fragment thereof.

The human peptide β-CGRP (UniProtKB/Swiss-Protref.: P10092.1) is encoded by the human gene CALCB (NCBI ref: NM_000728.4, NP_000719.1), and has the sequence: Ala-Cys-Asn-Thr-Ala-Thr-Cys-Val-Thr-His-Arg-Leu-Ala-Gly-Leu-Leu-Ser-Arg-Ser-Gly-Gly-Met-Val-Lys-Ser-Asn-Phe-Val-Pro-Thr-Asn-Val-Gly-Ser-Lys- Ala-Phe-NH2 (SEQ ID NO: 2). In certain example embodiments, the CGRP to be administered is human β-CGRP. In certain example embodiments, the human α-CGRP to be administered is SEQ ID NO: 2 or a functional variant or fragment thereof.

The gene name Areg or AREG may refer to the Amphiregulin gene or polypeptide according to NCBI Reference Sequence accession numbers NM_009704.4 or NM_001657.3. The gene name Calca or CALCA may refer to the Calcitonin/calcitonin-related polypeptide, alpha gene or polypeptide according to NCBI Reference Sequence accession numbers NM_001033954.3, NM_007587.2, NM_001033952.2, NM_001033953.2 or NM_001741.2. The gene name Ramp1 or RAMP1 may refer to the Receptor (calcitonin) activity modifying protein 1 gene or polypeptide according to NCBI Reference Sequence accession numbers NM_016894.3, NM_001168392.1, or NM_005855.3.

By functional variant or fragment of CGRP, it is herein referred to peptides which peptide sequence differ from the amino acid sequence of wild type CGRP, but that generally retains all the biological activity of CGRP. In certain embodiments, functional variants of CGRP are ligands binding to and activating the CGRP receptor. Functional variants may also include modified peptides, fusion proteins (e.g., fused to another protein, polypeptide or the like, such as an immunoglobulin or a fragment thereof), or peptides having non-natural amino acids. Functional variants may have an extended residence time in body fluids. In certain embodiments, a variant of CGRP has at least 80, 85, 90, 95, 99% of the biological activity of CGRP. In certain embodiments, a variant of α-CGRP has at least 80, 85, 90, 95, 99% of the biological activity of α-CGRP. In certain embodiments, a variant of β-CGRP has at least 80, 85, 90, 95, 99% of the biological activity of β-CGRP. Preferably, a functional variant of α-CGRP has at least 85%, at least 90%, at least 91%, at least 92%, at least 93%, at least 94%, at least 95%, at least 96%, at least 96%, at least 97%, at least 98%, at least 99% sequence identity with α-CGRP. Preferably, a functional variant of β-CGRP has at least 85%, at least 90%, at least 91%, at least 92%, at least 93%, at least 94%, at least 95%, at least 96%, at least 96%, at least 97%, at least 98%, at least 99% sequence identity with β-CGRP.

As used herein, the term “functional fragments” refers to a specific peptide that has a biological activity of interest, which peptide sequence is a part of the peptide sequence of the reference peptide, and that can be of any length, provided the biological activity of peptide of reference is retained by said fragment.

### Modulation Using a CGRP Receptor Agonist

In another aspect, methods of maintaining or inducing homeostasis of intestinal ILC2 cells may comprise administering a CGRP receptor agonist, or functional domain thereof, to a subject in need thereof. In certain example embodiments, a subject in need thereof may be a subject at risk for or having aberrant activation and expansion of intestinal ICL2 cells.

CGRP receptors have been described as heterodimeric molecules formed of the calcitonin receptor-like receptor (CRLR), linked to RAMP1 (CALCRL). RAMP1 is a transmembrane domain protein of the RAMP family, which further comprises RAMP2 and RAMP3. Several types of receptors are known that can be activated by CGRP: CGRP receptor (formed of CRLR and of RAMP1), AM₂ receptor (formed of CRLR and of RAMP3), and AMY₁ and AMY₃ receptors (formed of the calcitonin receptor and of RAMP1 and RAMP3, respectively). The CGRP receptors can therefore be distinguished from the AM₂, AMY₁ and AMY₃ receptors by the nature of the transmembrane domain of the RAMP family interacting with CRLR.

As used herein, “CGRP receptor”, refers to a protein receptor comprising the CRLR protein Ref NCBI: NP_005786.1), bound to the protein Receptor Activity Modifying Protein 1 (RAMP1) (Ref NCBI: NP_005846.1). Thus, CGRP receptors do not comprise the CRLR protein bound to RAMP2 or RAMP3.

### Modulation of One or More Biomarkers of an ILC2 Expression Signature

In certain embodiments, a method of maintaining or inducing homeostasis of intestinal ILC2 cells comprises administering or more agents capable of modulating expression, activity, or function of one or more biomarkers of the IL-25 inflammatory ILC2 gene signature defined in Table A. In another example embodiment, a method of maintaining or inducing homeostasis of intestinal ILC2 cells comprises administering or more agents capable of modulating expression, activity, or function of one or more biomarkers of the IL-25 inflammatory ILC2 gene signature defined at any one of [0058] to [0098].

### Modulation of a CGRP Signature

In certain embodiments, a method of maintaining of inducing homeostasis of intestinal ILC2 cells comprises administering one or more agents capable of modulating expression, activity, or function of one or more biomarkers of the CGRP signature defined at any one of [0109] to [0124].

### Modulation of Cell-Type Specific Biological Programs

In another aspect, embodiments disclosed herein provide a method of modulating an ILC2 inflammatory response comprising administering to a population of cells comprising ILC2s, mast cells, Th2 cells and/or fibroblasts one or more agents capable of modulating expression, activity of one or more signatures as defined in in any one of [0099] to [0107].

In one example embodiment, the method comprises administering to a population of cells comprising ILC2s, mast cells, Th2 cells and/or fibroblasts one or more agents capable of modulating expression, activity of one or more biological programs characterized by ILC Topic 2, wherein ILC Topic 2 comprises one or more genes or polypeptides selected from the group consisting of: Calca, Hs3st1, Areg, Il13, Il4, Ccl1, Hes1, Il17rb, Lgals7, Homer2, Il5, Gata3, Deptor, Ptpn13, Ly6a, Hba-a1, Kcnn4, Ccr4, Rxrg, Sub1, 1700061F12Rik, Cntnap2, AA467197, Ptgir, Il10, Nfkb1, Lmo4, Pparg, Plaur, Il9r, Serpine1, Scel, Bmp7, Neb, Sox8, Lpcat2, Samsn1, Alox5, Gpr65, Abhd17c, Gm20186, Gm973, Epas1, Ccr8, D430036J16Rik, Cd6, Stxbp6, 9230102O04Rik, Furin and Klf5.

In one example embodiment, the method comprises administering to a population of cells comprising ILC2s, mast cells, Th2 cells and/or fibroblasts one or more agents capable of modulating expression, activity of one or more biological programs characterized by myeloid cell Topic 1, wherein myeloid cell Topic 1 comprises one or more genes or polypeptides selected from the group consisting of: Cpa3, Cma1, Mcpt4, Tpsb2, Fcer1a, Hs3st1, Gata2, Cited4, Cyp11a1, Tph1, Furin, Rab27b, Slc45a3, Ccl1, Il13, Il1rl1, Itga2b, Cited2, Fam110c, Creb3l1, Rgs13, Tpsab1, Cyp26a1, Serpinb1a, Slc18a2, Gmpr, Rprm, Ero1l, Il4, Cd200r3, Glul, Kit, Lat, Alox5, Gchfr, mt-Atp6, Lat2, Prss34, Poln, Klk8, 4932438H23Rik, Slc6a13, Avil, Socs2, Smco4, Ier3, Lxn, Gpr171, Adk and Gata1.

In one example embodiment, the method comprises administering to a population of cells comprising ILC2s, mast cells, Th2 cells and/or fibroblasts one or more agents capable of modulating expression, activity of one or more biological programs characterized by T Cell Topic 5, wherein T cell Topic 5 comprises one or more genes or polypeptides selected from the group consisting of: 1700061F12Rik, Il13, Scin, Lgmn, Hlf, Smco4, Npnt, Il17rb, Deptor, Gata3, Gm2a, Il6, Il17a, Ltb4r1, Fgl2, Areg, Fbxl21, AA467197, Il1rl1, Me1, Gm5544, Tmem159, Rasgrp4, 1700012B07Rik, 1700113H08Rik, St6galnac5, Il4, Chdh, Slco2b1, Ccr9, Epas1, Grp, Lztfl1, Gm10369, Kif19a, Tenm4, Serpinf1, Gnb2, Ubox5, Plcl1, Rab31, Ffar2, Slx1b, Asb2, Zfp85, Tmsb4x, Hdc, Pxdc1, Heatr1 and Lgals7.

In one example embodiment, the method comprises administering to a population of cells comprising ILC2s, mast cells, Th2 cells and/or fibroblasts one or more agents capable of modulating expression, activity of one or more biological programs characterized by stromal cell Topic 4, wherein stromal cell Topic 4 comprises one or more genes or polypeptides selected from the group consisting of: Ccl21a, Cxcl13, Clu, Ccl19, Acta2, Mfge8, Apoe, Tagln, Cxcl1, Cilp, Ccl2, Il33, Cxcl12, Actg2, Serpina3n, Ccl7, Bst1, Serpina1a, Fmod, Grem1, Serpina1b, Slc36a2, Cnn1, Myh11, Art2b, Actc1, AI838599, Serpina1c, Cr2, Gxylt2, Crym, Dclk1, Serpina1d, Myl9, Parm1, Gm16685, Postn, Chrdl1, Colq, Csn2, Prss12, H2-M2, Trf, Sostdc1, Dsc3, Ctgf, Thbs4, Pcdh15, Rtn4r and A230065H16Rik.

In certain example embodiments, the agent suppresses one of the above biological programs, whereby an ILC2 inflammatory response is decreased. The one or more agents may comprise agent(s) that modulate the expression, activity or function of one or more genes of or polypeptides in ILC Topic 2, myeloid cell Topic 1, T cell Topic 5 or stromal cell Topic 4.

In certain example embodiments, the population of cells is in vivo. In certain embodiments, the in vivo population is present in the gut of a subject. In other example embodiments, the population of cell is an in vitro or ex vivo population of cells. In certain other example embodiments, the population of cells is an intestinal organoid.

### Modulation and Modulating Agents

As used herein, “modulating” or “to modulate” generally means either reducing or inhibiting the expression or activity of, or alternatively increasing the expression or activity of a target or antigen (e.g., CGRP). In particular, “modulating” or “to modulate” can mean either reducing or inhibiting the activity of, or alternatively increasing a (relevant or intended) biological activity of, a target or antigen as measured using a suitable in vitro, cellular or in vivo assay (which will usually depend on the target involved), by at least 5%, at least 10%, at least 25%, at least 50%, at least 60%, at least 70%, at least 80%, at least 90%, or more, compared to activity of the target in the same assay under the same conditions but without the presence of an agent. An “increase” or “decrease” refers to a statistically significant increase or decrease respectively. For the avoidance of doubt, an increase or decrease will be at least 10% relative to a reference, such as at least 10%, at least 20%, at least 30%, at least 40%, at least 50%, at least 60%, at least 70%, at least 80%, at least 90%, at least 95%, at least 97%, at least 98%, or more, up to and including at least 100% or more, in the case of an increase, for example, at least 2-fold, at least 3-fold, at least 4-fold, at least 5-fold, at least 6-fold, at least 7-fold, at least 8-fold, at least 9-fold, at least 10-fold, at least 50-fold, at least 100-fold, or more. “Modulating” can also involve effecting a change (which can either be an increase or a decrease) in affinity, avidity, specificity and/or selectivity of a target or antigen, such as CGRP. “Modulating” can also mean effecting a change with respect to one or more biological or physiological mechanisms, effects, responses, functions, pathways or activities in which the target or antigen (or in which its substrate(s), ligand(s) or pathway(s) are involved, such as its signaling pathway or metabolic pathway and their associated biological or physiological effects) is involved. Again, as will be clear to the skilled person, such an action as an agonist or an antagonist can be determined in any suitable manner and/or using any suitable assay known or described herein (e.g., in vitro or cellular assay), depending on the target or antigen involved.

Modulating can, for example, also involve allosteric modulation of the target and/or reducing or inhibiting the binding of the target to one of its substrates or ligands and/or competing with a natural ligand, substrate for binding to the target. Modulating can also involve activating the target or the mechanism or pathway in which it is involved. Modulating can for example also involve effecting a change in respect of the folding or conformation of the target, or in respect of the ability of the target to fold, to change its conformation (for example, upon binding of a ligand), to associate with other (sub)units, or to disassociate. Modulating can for example also involve effecting a change in the ability of the target to signal, phosphorylate, dephosphorylate, and the like.

As used herein, an “agent” can refer to a protein-binding agent that permits modulation of activity of proteins or disrupts interactions of proteins and other biomolecules, such as but not limited to disrupting protein-protein interaction, ligand-receptor interaction, or protein-nucleic acid interaction. Agents can also refer to DNA targeting or RNA targeting agents. Agents can also refer to a protein, such as CGRP. Agents may include a fragment, derivative and analog of an active agent. The terms “fragment,” “derivative” and “analog” when referring to polypeptides as used herein refers to polypeptides which either retain substantially the same biological function or activity as such polypeptides. An analog includes a proprotein which can be activated by cleavage of the proprotein portion to produce an active mature polypeptide. Such agents include, but are not limited to, antibodies (“antibodies” includes antigen-binding portions of antibodies such as epitope- or antigen-binding peptides, paratopes, functional CDRs; recombinant antibodies; chimeric antibodies; humanized antibodies; nanobodies; tribodies; midibodies; or antigen-binding derivatives, analogs, variants, portions, or fragments thereof), protein-binding agents, nucleic acid molecules, small molecules, recombinant protein, peptides, aptamers, avimers and protein-binding derivatives, portions or fragments thereof. An “agent” as used herein, may also refer to an agent that inhibits expression of a gene, such as but not limited to a DNA targeting agent (e.g., CRISPR system, TALE, Zinc finger protein) or RNA targeting agent (e.g., inhibitory nucleic acid molecules such as RNAi, miRNA, ribozyme).

In certain embodiments, the agent modulates CGRP signaling. In certain embodiments, the agent is an agonist or antagonist of CGRP receptor activity. The term “agonist of the CGRP receptor” may refer to a compound that binds to a CGRP receptor and activates said CGRP receptor (see, e.g., US Patent Publication No. 2016-0106813A1).

In certain embodiments, administration of CGRP provokes migraine attacks due to its vasodilation properties, which are associated with dilation of both the middle meningeal artery (MMA), a major artery that supplies blood to a membrane (dura) that envelops the brain, and the middle cerebral artery (MCA) (see, e.g., Silberstein et al., Fremanezumab for the Preventive Treatment of Chronic Migraine, N Engl J Med 2017; 377:2113-22). Several approaches are possible to diminish the potential side-effects of the compounds of the invention. These side-effects can be diminished by following a specific treatment scheme, more precisely by making sure that the consecutive administrations are separated by enough time without CGRP and/or agonist of the CGRP receptor treatment. In a particular embodiment, the consecutive administrations of CGRP and/or agonist of the CGRP receptor are separated by at least 1 day, preferably 2 days, yet preferably 5 days.

The composition of the invention can also advantageously be formulated in order to release CGRP and/or agonist of the CGRP receptor in the subject in a timely controlled fashion. In a particular embodiment, the composition of the invention is formulated for controlled release of CGRP and/or agonist of the CGRP receptor.

In certain embodiments, the agent is capable of inhibiting the CGRP receptor or blocking CGRP receptor interaction with CGRP. Such agents may also be referred to as CGRP receptor antagonists. In certain embodiments, CGRP receptor or CGRP expression is inhibited, e.g., by a DNA targeting agent (e.g., CRISPR system, TALE, Zinc finger protein) or an RNA targeting agent (e.g., inhibitory nucleic acid molecules). In some embodiments, CGRP receptor activity is inhibited. Such inhibition includes, e.g., reducing the expression of its ligand, CGRP, or by blocking the interaction of CGRP receptor with CGRP. In certain embodiments, the antagonist is an antibody or fragment thereof. In certain embodiments, the antibody is specific for CGRP or CGRP receptor.

The agents of the present invention may be modified, such that they acquire advantageous properties for therapeutic use (e.g., stability and specificity), but maintain their biological activity (see, also administration).

It is well known that the properties of certain proteins can be modulated by attachment of polyethylene glycol (PEG) polymers, which increases the hydrodynamic volume of the protein and thereby slows its clearance by kidney filtration. (See, e.g., Clark et al., J. Biol. Chem. 271: 21969-21977 (1996)). Therefore, it is envisioned that certain agents can be PEGylated (e.g., on peptide residues) to provide enhanced therapeutic benefits such as, for example, increased efficacy by extending half-life in vivo. In certain embodiments, PEGylation of the agents may be used to extend the serum half-life of the agents (e.g., CGRP) and allow for particular agents to be capable of crossing the blood-brain barrier. Thus, in one embodiment, PEGylating CGRP or the CGRP receptor agonists or antagonists improve the pharmacokinetics and pharmacodynamics of the CGRP receptor agonists or antagonists.

In regards to peptide PEGylation methods, reference is made to Lu et al., Int. J. Pept. Protein Res. 43: 127-38 (1994); Lu et al., Pept. Res. 6: 140-6 (1993); Felix et al., Int. J. Pept. Protein Res. 46: 253-64 (1995); Gaertner et al., Bioconjug. Chem. 7: 38-44 (1996); Tsutsumi et al., Thromb. Haemost. 77: 168-73 (1997); Francis et al., hit. J. Hematol. 68: 1-18 (1998); Roberts et al., J. Pharm. Sci. 87: 1440-45 (1998); and Tan et al., Protein Expr. Purif. 12: 45-52 (1998). Polyethylene glycol or PEG is meant to encompass any of the forms of PEG that have been used to derivatize other proteins, including, but not limited to, mono-(C1-10) alkoxy or aryloxy-polyethylene glycol. Suitable PEG moieties include, for example, 40 kDa methoxy poly(ethylene glycol) propionaldehyde (Dow, Midland, Mich.); 60 kDa methoxy poly(ethylene glycol) propionaldehyde (Dow, Midland, Mich.); 40 kDa methoxy poly(ethylene glycol) maleimido-propionamide (Dow, Midland, Mich.); 31 kDa alpha-methyl-w-(3-oxopropoxy), polyoxyethylene (NOF Corporation, Tokyo); mPEG2-NHS-40k (Nektar); mPEG2-MAL-40k (Nektar), SUNBRIGHT GL2-400MA ((PEG)240 kDa) (NOF Corporation, Tokyo), SUNBRIGHT ME-200MA (PEG20 kDa) (NOF Corporation, Tokyo). The PEG groups are generally attached to the peptide (e.g., CGRP) via acylation or alkylation through a reactive group on the PEG moiety (for example, a maleimide, an aldehyde, amino, thiol, or ester group) to a reactive group on the peptide (for example, an aldehyde, amino, thiol, a maleimide, or ester group).

The PEG molecule(s) may be covalently attached to any Lys, Cys, or K(CO(CH2)₂SH) residues at any position in a peptide. In certain embodiments, the CGRP receptor agonists described herein can be PEGylated directly to any amino acid at the N-terminus by way of the N-terminal amino group. A “linker arm” may be added to a peptide to facilitate PEGylation. PEGylation at the thiol side-chain of cysteine has been widely reported (see, e.g., Caliceti & Veronese, Adv. Drug Deliv. Rev. 55: 1261-77 (2003)). If there is no cysteine residue in the peptide, a cysteine residue can be introduced through substitution or by adding a cysteine to the N-terminal amino acid. In certain embodiments, CGRP receptor agonists are PEGylated through the side chains of a cysteine residue added to the N-terminal amino acid.

In exemplary embodiments, the PEG molecule(s) may be covalently attached to an amide group in the C-terminus of a peptide, such as in the CGRP receptor agonist. In preferred embodiments, there is at least one PEG molecule covalently attached to the CGRP receptor agonist. In certain embodiments, the PEG molecule used in modifying an agent of the present invention is branched while in other embodiments, the PEG molecule may be linear. In particular aspects, the PEG molecule is between 1 kDa and 100 kDa in molecular weight. In further aspects, the PEG molecule is selected from 10, 20, 30, 40, 50, 60, and 80 kDa. In further still aspects, it is selected from 20, 40, or 60 kDa. Where there are two PEG molecules covalently attached to the agent of the present invention, each is 1 to 40 kDa and in particular aspects, they have molecular weights of 20 and 20 kDa, 10 and 30 kDa, 30 and 30 kDa, 20 and 40 kDa, or 40 and 40 kDa. In particular aspects, the agent (e.g., CGRP receptor agonists or antagonists) contain mPEG-cysteine. The mPEG in mPEG-cysteine can have various molecular weights. The range of the molecular weight is preferably 5 kDa to 200 kDa, more preferably 5 kDa to 100 kDa, and further preferably 20 kDa to 60 kDA. The mPEG can be linear or branched.

In particular embodiments, the agents (e.g., CGRP, or CGRP agonist or antagonists) include a protecting group covalently joined to the N-terminal amino group. In exemplary embodiments, a protecting group covalently joined to the N-terminal amino group of the CGRP receptor agonists reduces the reactivity of the amino terminus under in vivo conditions. Amino protecting groups include —C1-10 alkyl, —C1-10 substituted alkyl, —C2-10 alkenyl, —C2-10 substituted alkenyl, aryl, —C1-6 alkyl aryl, —C(O)—(CH2)1-6-COOH, —C(O)—C1-6 alkyl, —C(O)-aryl, —C(O)—O—C1-6 alkyl, or —C(O)—O-aryl. In particular embodiments, the amino terminus protecting group is selected from the group consisting of acetyl, propyl, succinyl, benzyl, benzyloxycarbonyl, and t-butyloxycarbonyl. In other embodiments, deamination of the N-terminal amino acid is another modification that may be used for reducing the reactivity of the amino terminus under in vivo conditions.

Chemically modified compositions of the agents (e.g., CGRP, or CGRP receptor agonists or antagonists) wherein the agent is linked to a polymer are also included within the scope of the present invention. The polymer selected is usually modified to have a single reactive group, such as an active ester for acylation or an aldehyde for alkylation, so that the degree of polymerization may be controlled. Included within the scope of polymers is a mixture of polymers. Preferably, for therapeutic use of the end-product preparation, the polymer will be pharmaceutically acceptable. The polymer or mixture thereof may include but is not limited to polyethylene glycol (PEG), monomethoxy-polyethylene glycol, dextran, cellulose, or other carbohydrate-based polymers, poly-(N-vinyl pyrrolidone) polyethylene glycol, propylene glycol homopolymers, a polypropylene oxide/ethylene oxide co-polymer, polyoxyethylated polyols (for example, glycerol), and polyvinyl alcohol.

In other embodiments, the agents (e.g., CGRP receptor agonists or antagonists) are modified by PEGylation, cholesterylation, or palmitoylation. The modification can be to any amino acid residue. In preferred embodiments, the modification is to the N-terminal amino acid of the agent (e.g., CGRP receptor agonist or antagonists), either directly to the N-terminal amino acid or by way coupling to the thiol group of a cysteine residue added to the N-terminus or a linker added to the N-terminus such as trimesoyl tris(3,5-dibromosalicylate (Ttds). In certain embodiments, the N-terminus of the agent (e.g., CGRP receptor agonist or antagonist) comprises a cysteine residue to which a protecting group is coupled to the N-terminal amino group of the cysteine residue and the cysteine thiolate group is derivatized with N-ethylmaleimide, PEG group, cholesterol group, or palmitoyl group. In other embodiments, an acetylated cysteine residue is added to the N-terminus of the agents, and the thiol group of the cysteine is derivatized with N-ethylmaleimide, PEG group, cholesterol group, or palmitoyl group. In certain embodiments, the agent of the present invention is a conjugate. In certain embodiments, the agent of the present invention (e.g., CGRP receptor agonists or antagonists) is a polypeptide consisting of an amino acid sequence which is bound with a methoxypolyethylene glycol(s) via a linker.

Substitutions of amino acids may be used to modify an agent of the present invention. The phrase “substitution of amino acids” as used herein encompasses substitution of amino acids that are the result of both conservative and non-conservative substitutions. Conservative substitutions are the replacement of an amino acid residue by another similar residue in a polypeptide. Typical but not limiting conservative substitutions are the replacements, for one another, among the aliphatic amino acids Ala, Val, Leu and Ile; interchange of Ser and Thr containing hydroxy residues, interchange of the acidic residues Asp and Glu, interchange between the amide-containing residues Asn and Gln, interchange of the basic residues Lys and Arg, interchange of the aromatic residues Phe and Tyr, and interchange of the small-sized amino acids Ala, Ser, Thr, Met, and Gly. Non-conservative substitutions are the replacement, in a polypeptide, of an amino acid residue by another residue which is not biologically similar. For example, the replacement of an amino acid residue with another residue that has a substantially different charge, a substantially different hydrophobicity, or a substantially different spatial configuration.

In certain embodiments, the present invention provides for one or more therapeutic agents. In certain embodiments, the one or more agents comprises a small molecule inhibitor, small molecule degrader (e.g., PROTAC), genetic modifying agent, antibody, antibody fragment, antibody-like protein scaffold, aptamer, protein, or any combination thereof.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse). In certain embodiments, the present invention provides for one or more therapeutic agents against combinations of targets identified. Targeting the identified combinations may provide for enhanced or otherwise previously unknown activity in the treatment of disease.

### Small Molecules

In certain embodiments, the one or more agents is a small molecule. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking a binding site or activating a receptor by binding to a ligand binding site).

One type of small molecule applicable to the present invention is a degrader molecule. Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481; Bondeson and Crews, Targeted Protein Degradation by Small Molecules, Annu Rev Pharmacol Toxicol. 2017 Jan. 6; 57: 107-123; and Lai et al., Modular PROTAC Design for the Degradation of Oncogenic BCR-ABL Angew Chem Int Ed Engl. 2016 Jan. 11; 55(2): 807-810).

In certain embodiments, combinations of targets are modulated (e.g., CGRP and one or more targets related to a gene signature gene). In certain embodiments, an agent against one of the targets in a combination may already be known or used clinically. In certain embodiments, targeting the combination may require less of the agent as compared to the current standard of care and provide for less toxicity and improved treatment.

### GPR65

In certain example embodiments, the agent is an agent that modulates GPR65, also known as T cell death-associated gene 8 (TDAG8) is a G protein-coupled receptor (GPCR) protein that in humans is encoded by the GPR65 gene. GPR65 senses extracellular pH. It was found that cAMP levels increased when GPR65 was stimulated by pH values less than pH 7.2. Recent studies disclose that TDAG8 (GPR65) inhibits intestinal inflammation in the dss-induced experimental colitis mouse model (Sanderlin, et al., 2018, TDAG8 (GPR65) Inhibits Intestinal Inflammation in the DSS-Induced Experimental Colitis Mouse Model, bioRxiv 496315; doi.org/10.1101/496315). TDAG8-null mice showed exacerbation of intestinal inflammation and fibrosis. id. Aberrant TDAG8 function is associated with IBD development and progression (Jostins, et al., Host-microbe interactions have shaped the genetic architecture of inflammatory bowel disease. Nature 491: 119-124, 2012; and Lassen, et al., Genetic Coding Variant in GPR65 Alters Lysosomal pH and Links Lysosomal Dysfunction with Colitis Risk. Immunity, 2016). Applicants have shown for the first time that CGRP induces expression of GPR65 in ILC2s and suppresses type 2 inflammation-induced activation and expansion of intestinal ILC2s through activation of a cAMP response module. In certain embodiments, combination treatment with GPR65 agonists and agonists of CGRP signaling (e.g., CGRP) can be used in the treatment of or prevention of ILC2 inflammatory responses.

A GPR65 agonist, BTB09089 ((3-[(2,4-dichlorobenzyl)thio]-1,6-dimethyl-5,6-dihydro-1H-pyridazino[4,5-e][1,3,4]thiadiazin-5-one), has been developed and recently investigated for anti-inflammatory properties. In one study, BTB09089 was shown to activate TDAG8 in vitro (Onozawa, et al., Activation of T cell death associated gene 8 regulates the cytokine production of T cells and macrophages in vitro. Eur J Pharmacol 683: 325-331, 2012). An additional study has shown in vivo efficacy of BTB09089 using an ischemic stroke murine disease model (Ma et al., TDAG8 activation attenuates cerebral ischaemia-reperfusion injury via Akt signalling in rats. Exp Neurol 293: 115-123, 2017). In certain embodiments, CGRP is administered in combination with BTB09089 or similar molecules (see, e.g., pubchem.ncbi.nlm.nih.gov/compound/2801217). Additionally, allosteric agonists and negative allosteric modulators (NAMs) for GPR65 applicable to the present invention have been identified (ZINC62678696) (see, e.g., Huang, et al., Allosteric ligands for the pharmacologically dark receptors GPR68 and GPR65, Nature. 2015 Nov. 26; 527(7579): 477-483).

### PDCD1

PDCD1 is the human gene encoding the immune checkpoint protein PD-1. Immune checkpoints are regulators of the immune system. These pathways are crucial for self-tolerance, which prevents the immune system from attacking cells indiscriminately. Modulating immune checkpoint activity in response to upregulation by CGRP may reduce an ILC2 inflammatory response or maintain homeostasis. In certain embodiments, a combination treatment may include CGRP and a checkpoint agonist. Checkpoint proteins may include TIM3, CTLA4, or PD-1. Immune checkpoint agonists may activate checkpoint signaling, for example, by binding to the checkpoint protein. The agonists may include a ligand (e.g., PD-L1). PD-1 agonist antibodies that mimic PD-1 ligand (PD-L1) have been described (see, e.g., US20170088618A1; WO2018053405A1). Such agonist antibodies against any receptor described herein are applicable to the present invention.

Adenylate Cyclase Activators

In certain embodiments, the one or more modulating agents comprises an adenylate cyclase activator. The adenylate cyclase activator may be forskolin, Other non-limiting examples of adenylate cyclase activators applicable to present invention include forskolin derivatives, an extract of Coleus forskohlii having adenylate cyclase activator activity, carbacyclin, isoproterenol, prostaglandin D₂, prostaglandin Ei and prostaglandin I₂ (prostacyclin). As used herein, “Forskolin” refers to a labdane diterpene that is produced by the Indian Coleus plant (Coleus forskohlii, aka Plectranthus barbatus). Forskolin is commonly used to raise levels of cyclic AMP (cAMP) in the study and research of cell physiology. A number of structural variants of forskolin are known in the art and may be referred to herein as forskolin derivatives, for example those described in Kokic, Curr Med Chem Cardiovasc Hematol Agents. 2005 October; 3(4):333-9; Gao et al, Mini Rev Med Chem. 2005 June; 5(6):545-53; Head, Altern Med Rev. 2001 April; 6(2): 141-66; Zidek, Eur Cytokine Netw. 2001 March; 12(1):22-32; Ong et al., Acta Pharmacol Sin. 2000 February; 21(2):111-23; Chen et al., Lab Invest. 1998 February; 78(2):165-74; Milligan et al., Receptors Channels. 1997; 5(3-4):209-13; Sulakhe et al. Mol Cell Biochem. 1995 August-September; 149-150:103-26; Ehlert et al., Life Sci. 1995; 56(11-12):965-71; and Farah et al., Annu Rev Pharmacol Toxicol. 1984; 24:275-328. Preparation, including solubilization, and use of forskolin and related compounds including forskolin derivatives, are described in, e.g., U.S. Pat. No. 6,960,300 and in Chen et al., 2009 J. Nat. Prod. 72:769. Adenylate cyclase activators may also include those disclosed in U.S. Pat. No. 6,333,354.

### Antibodies

The term “antibody” (e.g., anti-CGRP or anti-CGRP receptor antibody) is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, V_{HH} and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g. the IgG1, IgG2, IgG3, and IgG4 subclassess of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, lgM antibodies exist in pentameric form, and IgA antibodies exist in monomeric, dimeric or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g. LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins-harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, V_{H} and CH1 domains; (ii) the Fab′ fragment, which is a Fab fragment having one or more cysteine residues at the C-terminus of the C_{H}1 domain; (iii) the Fd fragment having VH and CH1 domains; (iv) the Fd′ fragment having V_{H} and CH1 domains and one or more cysteine residues at the C-terminus of the CHI domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii) isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; International Patent Publication No. WO 93/11161; Hollinger et al., 90 PNAS 6444 (1993)); (xi) “linear antibodies” comprising a pair of tandem Fd segments (V_{H}-Cₕ1-V_{H}-Cₕ1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds. For example, an antagonist antibody may bind CGRP receptor or CGRP and inhibit the ability to suppress an ILC class 2 inflammatory response. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., International Patent Publication No. WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

The disclosure also encompasses nucleic acid molecules, in particular those that inhibit CGRP receptor or CGRP. Exemplary nucleic acid molecules include aptamers, siRNA, artificial microRNA, interfering RNA or RNAi, dsRNA, ribozymes, antisense oligonucleotides, and DNA expression cassettes encoding said nucleic acid molecules. Preferably, the nucleic acid molecule is an antisense oligonucleotide. Antisense oligonucleotides (ASO) generally inhibit their target by binding target mRNA and sterically blocking expression by obstructing the ribosome. ASOs can also inhibit their target by binding target mRNA thus forming a DNA-RNA hybrid that can be a substance for RNase H. Preferred ASOs include Locked Nucleic Acid (LNA), Peptide Nucleic Acid (PNA), and morpholinos Preferably, the nucleic acid molecule is an RNAi molecule, i.e., RNA interference molecule. Preferred RNAi molecules include siRNA, shRNA, and artificial miRNA. The design and production of siRNA molecules is well known to one of skill in the art (e.g., Hajeri P B, Singh S K. Drug Discov Today. 2009 14(17-18):851-8). The nucleic acid molecule inhibitors may be chemically synthesized and provided directly to cells of interest. The nucleic acid compound may be provided to a cell as part of a gene delivery vehicle. Such a vehicle is preferably a liposome or a viral gene delivery vehicle.

### Genetic Modifying Agents

In certain embodiments, the one or more modulating agents may be a genetic modifying agent. The genetic modifying agent may comprise a CRISPR system, a zinc finger nuclease system, a TALEN, a meganuclease or RNAi system. The genetic modifying agent preferably modulates expression of Gpr65, Pdcd1, Crem, Egln3, Adora2a, Rgs2, Gna15, Adrb2, Gadd45a, Areg, Hif1a, Dusp1, Pde4b, Cdkn1a, Akap12 and Il5, preferably, GPR65 and/or PD-1 (e.g., guide sequences designed to target the genes). In certain embodiments, CGRP induces expression of Gpr65, Pdcd1, Crem, Egln3, Adora2a, Rgs2, Gna15, Adrb2, Gadd45a, Areg, Hif1a, Dusp1, Pde4b, Cdkn1a, Akap12 and Il5 to maintain homeostasis of intestinal ILC2 cells. The genetic modifying agent also preferably modulates expression of a cAMP response module, wherein the cAMP module comprises one or more genes selected from the group consisting of: Adrb2, Adora2a, Pde4b, Akap12, Areg, Crem and Il5. The genetic modifying agent also preferably modulates expression of one or more genes in one or more biological programs characterized by ILC Topic 2, myeloid cell Topic 1, T cell Topic 5 or stromal cell Topic 4, wherein ILC Topic 2 comprises one or more genes or polypeptides selected from the group consisting of: Calca, Hs3st1, Areg, Il13, Il4, Ccl1, Hes1, Il17rb, Lgals7, Homer2, Il5, Gata3, Deptor, Ptpn13, Ly6a, Hba-a1, Kcnn4, Ccr4, Rxrg, Sub1, 1700061F12Rik, Cntnap2, AA467197, Ptgir, Il10, Nfkb1, Lmo4, Pparg, Plaur, Il9r, Serpine1, Scel, Bmp7, Neb, Sox8, Lpcat2, Samsn1, Alox5, Gpr65, Abhd17c, Gm20186, Gm973, Epas1, Ccr8, D430036J16Rik, Cd6, Stxbp6, 9230102O04Rik, Furin and Klf5, wherein myeloid cell Topic 1 comprises one or more genes or polypeptides selected from the group consisting of: Cpa3, Cma1, Mcpt4, Tpsb2, Fcer1a, Hs3st1, Gata2, Cited4, Cyp11a1, Tph1, Furin, Rab27b, Slc45a3, Ccl1, Il13, Il1rl1, Itga2b, Cited2, Fam110c, Creb3l1, Rgs13, Tpsab1, Cyp26a1, Serpinb1a, Slc18a2, Gmpr, Rprm, Ero1l, Il4, Cd200r3, Glul, Kit, Lat, Alox5, Gchfr, mt-Atp6, Lat2, Prss34, Poln, Klk8, 4932438H23Rik, Slc6a13, Avil, Socs2, Smco4, Ier3, Lxn, Gpr171, Adk and Gata1, wherein T cell Topic 5 comprises one or more genes or polypeptides selected from the group consisting of: 1700061F12Rik, Il13, Scin, Lgmn, Hlf, Smco4, Npnt, Il17rb, Deptor, Gata3, Gm2a, Il6, Il17a, Ltb4r1, Fgl2, Areg, Fbxl21, AA467197, Il1rl1, Me1, Gm5544, Tmem159, Rasgrp4, 1700012B07Rik, 1700113H08Rik, St6galnac5, Il4, Chdh, Slco2b1, Ccr9, Epas1, Grp, Lztfl1, Gm10369, Kif19a, Tenm4, Serpinf1, Gnb2, Ubox5, Plcl1, Rab31, Ffar2, Slx1b, Asb2, Zfp85, Tmsb4x, Hdc, Pxdc1, Heatr1 and Lgals7, wherein stromal cell Topic 4 comprises one or more genes or polypeptides selected from the group consisting of: Ccl21a, Cxcl13, Clu, Ccl19, Acta2, Mfge8, Apoe, Tagln, Cxcl1, Cilp, Ccl2, Il33, Cxcl12, Actg2, Serpina3n, Ccl7, Bst1, Serpina1a, Fmod, Grem1, Serpina1b, Slc36a2, Cnn1, Myh11, Art2b, Actc1, AI838599, Serpina1c, Cr2, Gxylt2, Crym, Dclk1, Serpina1d, Myl9, Parm1, Gm16685, Postn, Chrdl1, Colq, Csn2, Prss12, H2-M2, Trf, Sostdc1, Dsc3, Ctgf, Thbs4, Pcdh15, Rtn4r and A230065H16Rik. In certain embodiments, the one or more biological programs are suppressed, whereby an ILC2 inflammatory response is decreased.

### CRISPR-Cas Modification

In some embodiments, a polynucleotide of the present invention described elsewhere herein can be modified using a CRISPR-Cas and/or Cas-based system.

In general, a CRISPR-Cas or CRISPR system as used in herein and in documents, such as International Patent Publication No. WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molcel.2015.10.008.

CRISPR-Cas systems can generally fall into two classes based on their architectures of their effector molecules, which are each further subdivided by type and subtype. The two class are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein.

In some embodiments, the CRISPR-Cas system that can be used to modify a polynucleotide of the present invention described herein can be a Class 1 CRISPR-Cas system. In some embodiments, the CRISPR-Cas system that can be used to modify a polynucleotide of the present invention described herein can be a Class 2 CRISPR-Cas system.

### Class 1 CRISPR-Cas Systems

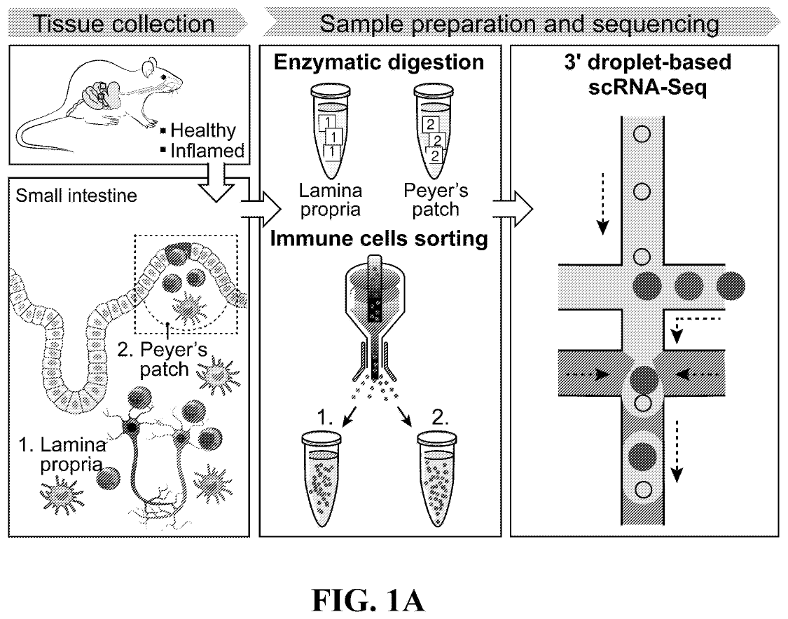

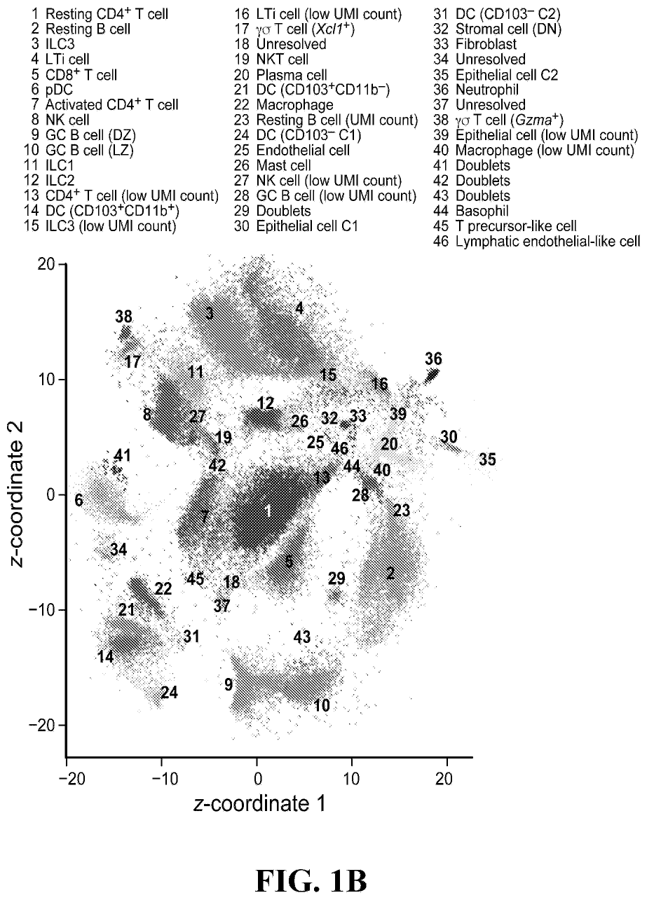

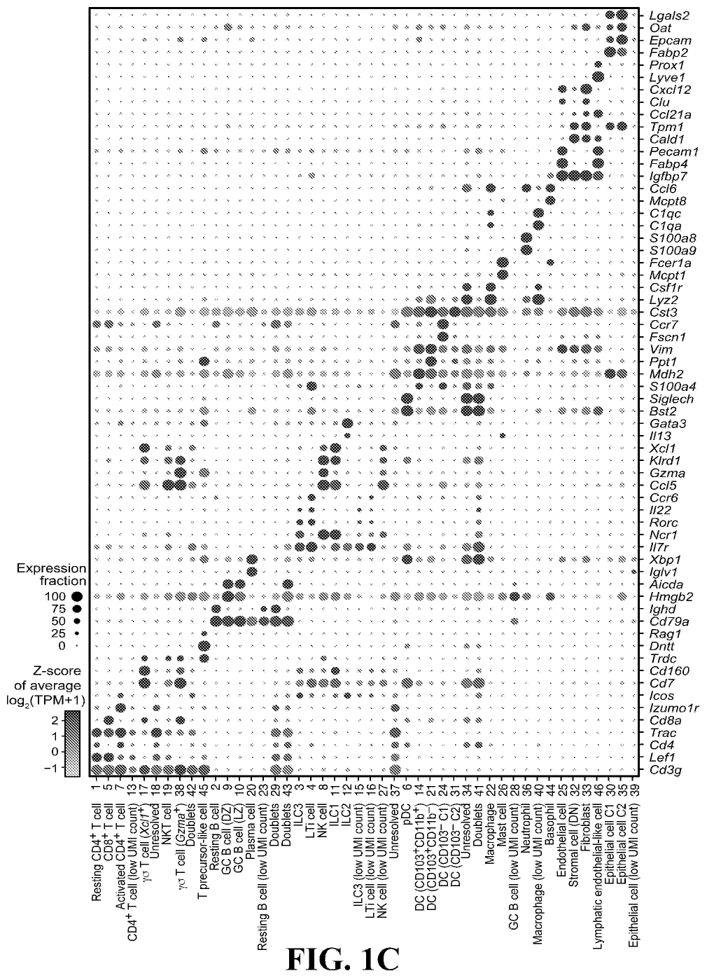

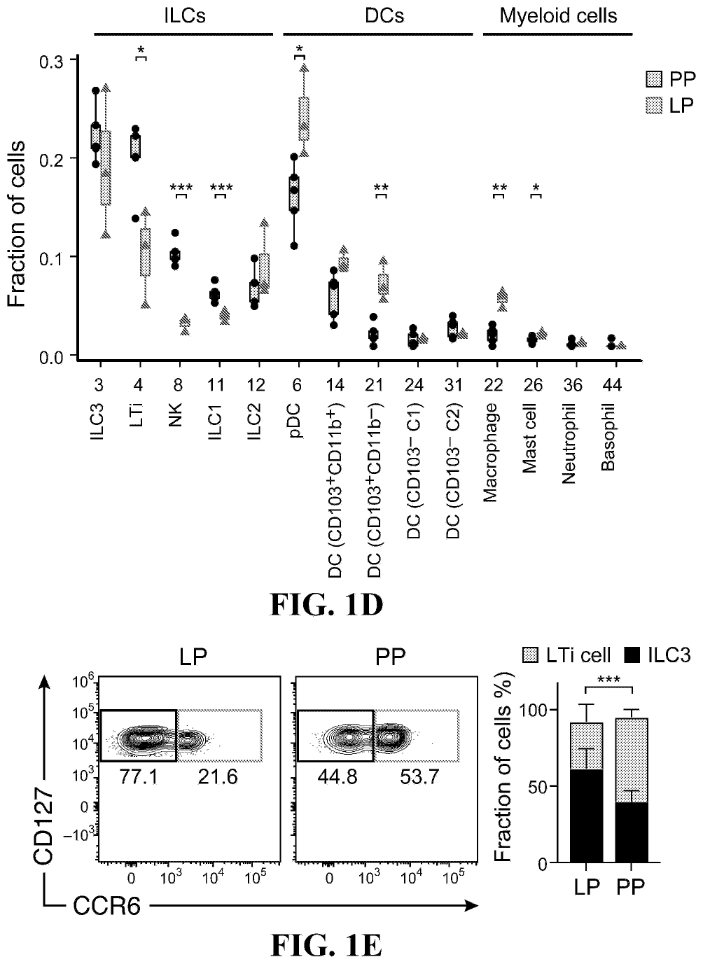

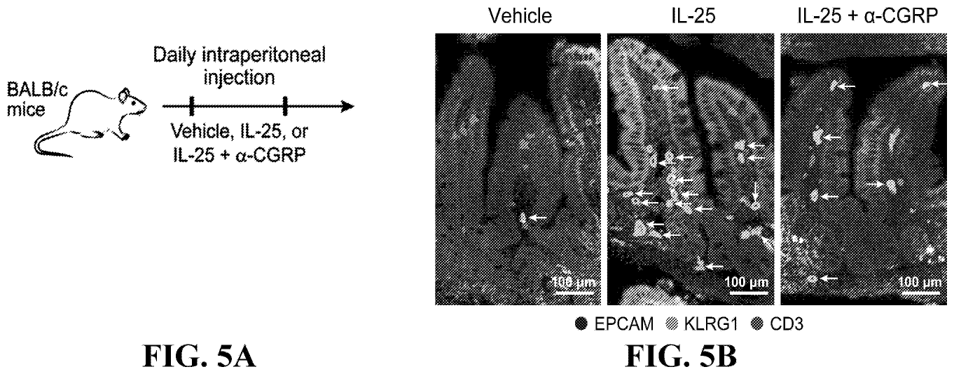

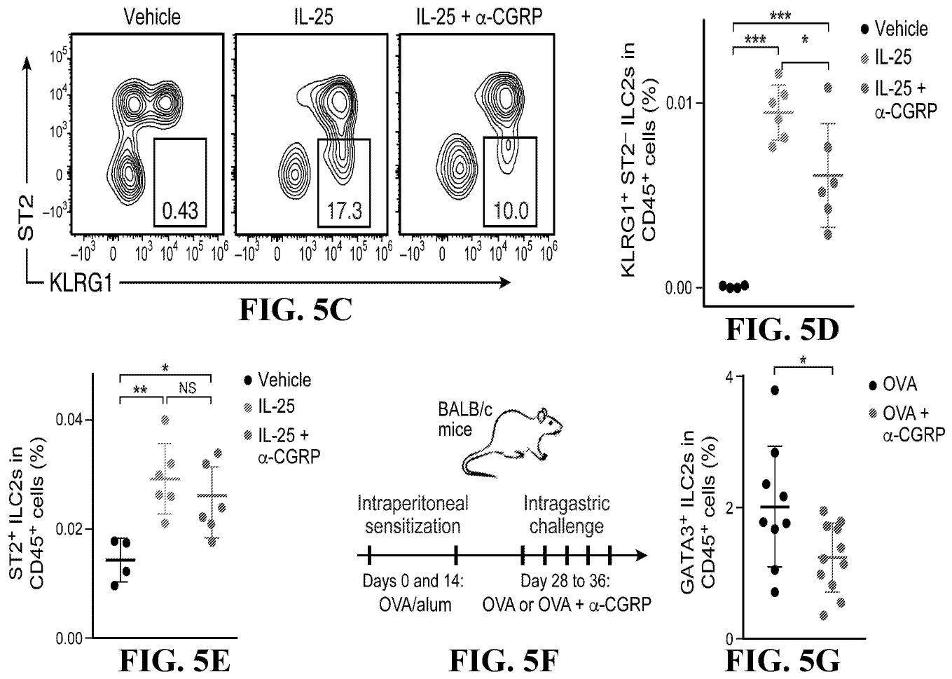

In some embodiments, the CRISPR-Cas system that can be used to modify a polynucleotide of the present invention described herein can be a Class 1 CRISPR-Cas system. Class 1 CRISPR-Cas systems are divided into types I, II, and IV. Makarova et al. 2020. Nat. Rev. 18: 67-83., particularly as described in FIG. 1. Type I CRISPR-Cas systems are divided into 9 subtypes (I-A, I-B, I-C, I-D, I-E, I-F1, I-F2, I-F3, and IG). Makarova et al., 2020. Class 1, Type I CRISPR-Cas systems can contain a Cas3 protein that can have helicase activity. Type III CRISPR-Cas systems are divided into 6 subtypes (III-A, III-B, III-E, and III-F). Type III CRISPR-Cas systems can contain a Cas10 that can include an RNA recognition motif called Palm and a cyclase domain that can cleave polynucleotides. Makarova et al., 2020. Type IV CRISPR-Cas systems are divided into 3 subtypes. (IV-A, IV-B, and IV-C). Makarova et al., 2020. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al. 2018. The CRISPR Journal, v. 1, n5, FIG. 5.

The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAMPs) family subunits (e.g., Cas 5, Cas6, and/or Cas7). RAMP proteins are characterized by having one or more RNA recognition motif domains. In some embodiments, multiple copies of RAMPs can be present. In some embodiments, the Class I CRISPR-Cas system can include 1, 2, 3, 4, 5, 6, 7, 8, 9, 10, 11, 12 or more Cas5, Cas6, and/or Cas 7 proteins. In some embodiments, the Cas6 protein is an RNAse, which can be responsible for pre-crRNA processing. When present in a Class 1 CRISPR-Cas system, Cas6 can be optionally physically associated with the effector complex.

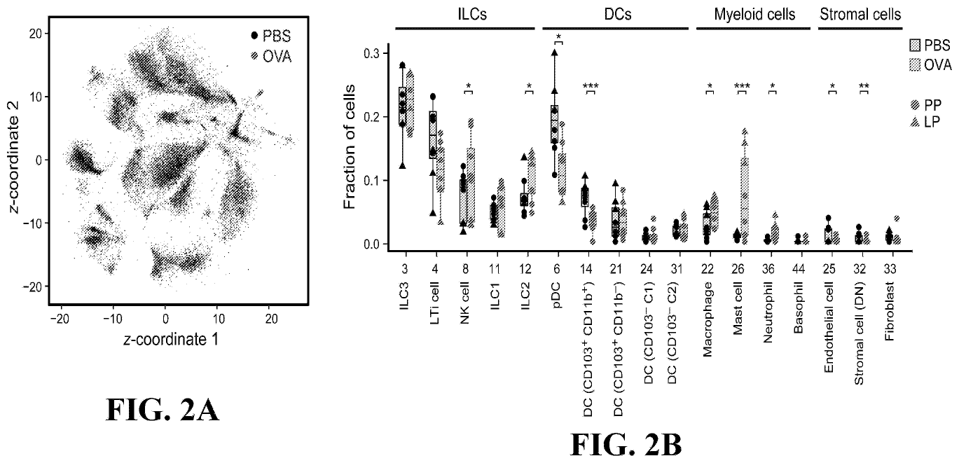

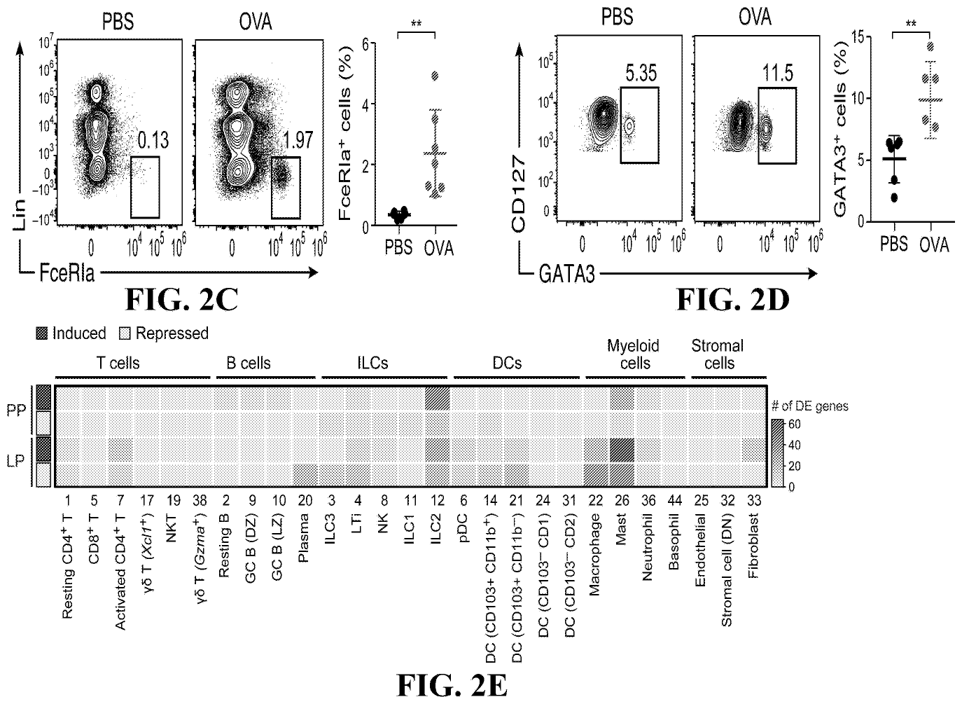

Class 1 CRISPR-Cas system effector complexes can, in some embodiments, also include a large subunit. The large subunit can be composed of or include a Cas8 and/or Cas10 protein. See, e.g., FIGS. 1 and 2. Koonin E V, Makarova K S. 2019. Phil. Trans. R. Soc. B 374: 20180087, DOI: 10.1098/rstb.2018.0087 and Makarova et al. 2020.

Class 1 CRISPR-Cas system effector complexes can, in some embodiments, include a small subunit (for example, Cash 1). See, e.g., FIGS. 1 and 2. Koonin E V, Makarova K S. 2019 Origins and Evolution of CRISPR-Cas systems. Phil. Trans. R. Soc. B 374: 20180087, DOI: 10.1098/rstb.2018.0087.

In some embodiments, the Class 1 CRISPR-Cas system can be a Type I CRISPR-Cas system. In some embodiments, the Type I CRISPR-Cas system can be a subtype I-A CRISPR-Cas system. In some embodiments, the Type I CRISPR-Cas system can be a subtype I-B CRISPR-Cas system. In some embodiments, the Type I CRISPR-Cas system can be a subtype I-C CRISPR-Cas system. In some embodiments, the Type I CRISPR-Cas system can be a subtype I-D CRISPR-Cas system. In some embodiments, the Type I CRISPR-Cas system can be a subtype I-E CRISPR-Cas system. In some embodiments, the Type I CRISPR-Cas system can be a subtype I-F1 CRISPR-Cas system. In some embodiments, the Type I CRISPR-Cas system can be a subtype I-F2 CRISPR-Cas system. In some embodiments, the Type I CRISPR-Cas system can be a subtype I-F3 CRISPR-Cas system. In some embodiments, the Type I CRISPR-Cas system can be a subtype I-G CRISPR-Cas system. In some embodiments, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems as previously described.

In some embodiments, the Class 1 CRISPR-Cas system can be a Type III CRISPR-Cas system. In some embodiments, the Type III CRISPR-Cas system can be a subtype III-A CRISPR-Cas system. In some embodiments, the Type III CRISPR-Cas system can be a subtype III-B CRISPR-Cas system. In some embodiments, the Type III CRISPR-Cas system can be a subtype III-C CRISPR-Cas system. In some embodiments, the Type III CRISPR-Cas system can be a subtype III-D CRISPR-Cas system. In some embodiments, the Type III CRISPR-Cas system can be a subtype III-E CRISPR-Cas system. In some embodiments, the Type III CRISPR-Cas system can be a subtype III-F CRISPR-Cas system.

In some embodiments, the Class 1 CRISPR-Cas system can be a Type IV CRISPR-Cas-system. In some embodiments, the Type IV CRISPR-Cas system can be a subtype IV-A CRISPR-Cas system. In some embodiments, the Type IV CRISPR-Cas system can be a subtype IV-B CRISPR-Cas system. In some embodiments, the Type IV CRISPR-Cas system can be a subtype IV-C CRISPR-Cas system.

The effector complex of a Class 1 CRISPR-Cas system can, in some embodiments, include a Cas3 protein that is optionally fused to a Cas2 protein, a Cas4, a Cas5, a Cash, a Cas7, a Cas8, a Cas10, a Cas11, or a combination thereof. In some embodiments, the effector complex of a Class 1 CRISPR-Cas system can have multiple copies, such as 1, 2, 3, 4, 5, 6, 7, 8, 9, 10, 11, 12, 13, or 14, of any one or more Cas proteins.

### Class 2 CRISPR-Cas Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), CasX, and/or Cas14.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SETT/9), a translation initiation domain, a transcriptional repression domain (e.g., a KRAB domain, NuE domain, NcoR domain, and a SID domain such as a SID4X domain), a nuclease domain (e.g., Fold), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (WO 2019/005884, WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Application Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33(2): 139-142 and WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C•G base pair into a T•A base pair (Komor et al. 2016. Nature. 533:420-424; Nishida et al. 2016. Science. 353; and Li et al. Nat. Biotech. 36:324-327) and ABEs convert an A•T base pair to a G•C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471.

Other Example Type V base editing systems are described in WO 2018/213708, WO 2018/213726, PCT/US2018/067207, PCT/US2018/067225, and PCT/US2018/067307 which are incorporated by referenced herein.

In certain example embodiments, the base editing system may be a RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA based editor may be used to delete or introduce a post-translation modification site in the expressed mRNA. In contrast to DNA base editors, whose edits are permanent in the modified cell, RNA base editors can provide edits where finer temporal control may be needed, for example in modulating a particular immune response. Example Type VI RNA-base editing systems are described in Cox et al. 2017. Science 358: 1019-1027, WO 2019/005884, WO 2019/005886, WO 2019/071048, PCT/US20018/05179, PCT/US2018/067207, which are incorporated herein by reference. An example FnCas9 system that may be adapted for RNA base editing purposes is described in WO 2016/106236, which is incorporated herein by reference.

An example method for delivery of base-editing systems, including use of a split-intein approach to divide CBE and ABE into reconstituble halves, is described in Levy et al. Nature Biomedical Engineering doi.org/10.1038/s41441-019-0505-5 (2019), which is incorporated herein by reference.

### Prime Editors

In some embodiments, a polynucleotide of the present invention described elsewhere herein can be modified using a prime editing system See e.g. Anzalone et al. 2019. Nature. 576: 149-157. Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion, and combinations thereof. Generally, a prime editing system, as exemplified by PE1, PE2, and PE3 (Id.), can include a reverse transcriptase fused or otherwise coupled or associated with an RNA-programmable nickase, and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g. sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g. a prime editing guide molecule or peg guide molecule) directly into the target site in the target polynucleotide. See e.g. Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g. is a Cas9 nickase). In some embodiments, the Cas polypeptide is fused to the reverse transcriptase. In some embodiments, the Cas polypeptide is linked to the reverse transcriptase.

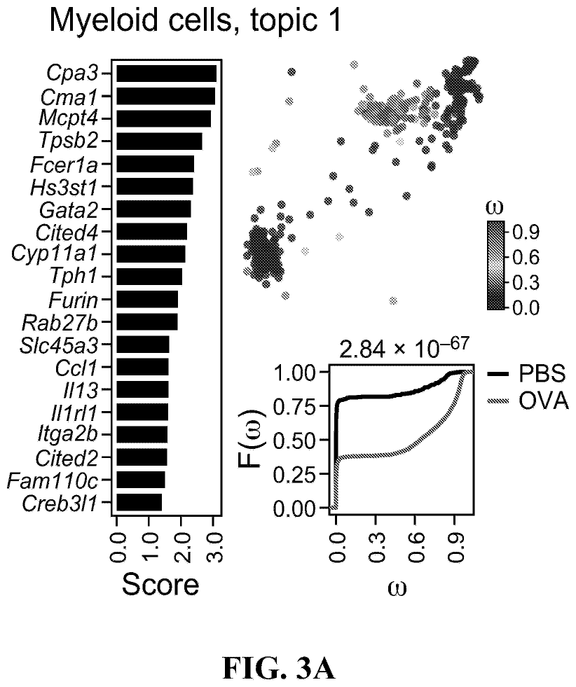

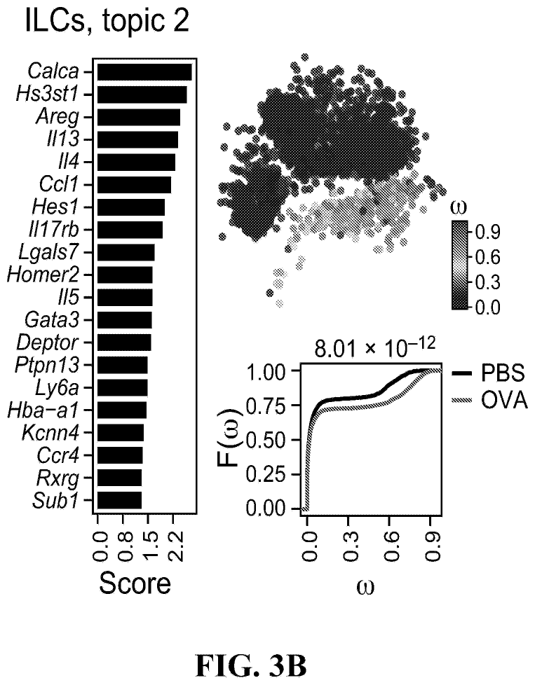

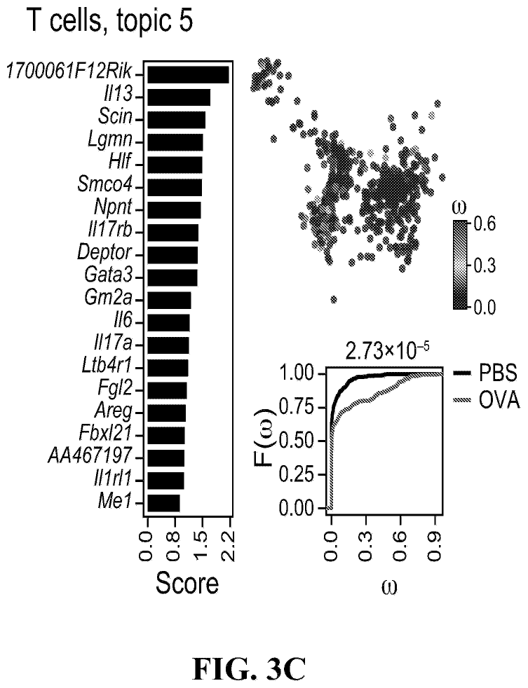

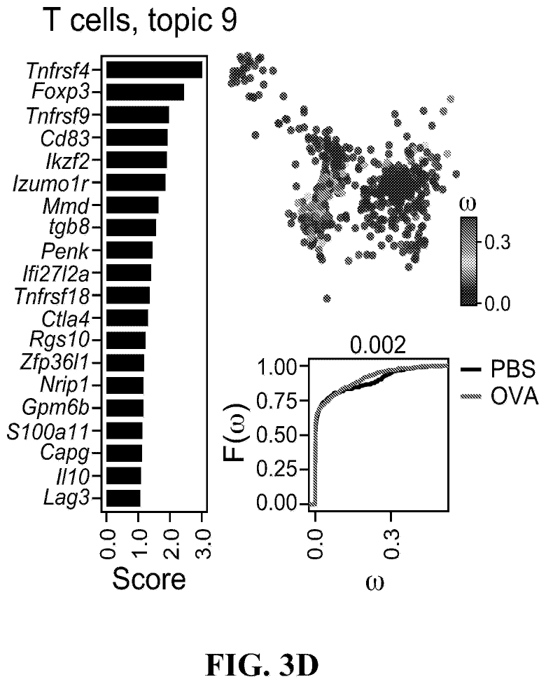

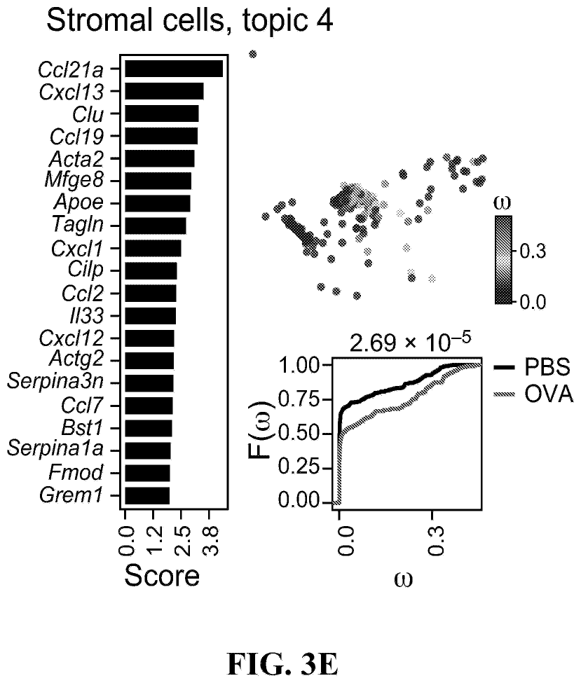

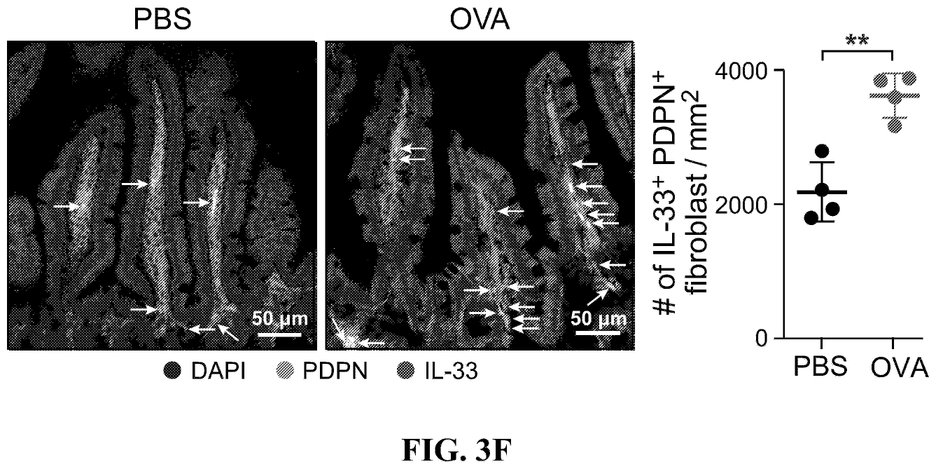

In some embodiments, the prime editing system can be a PE1 system or variant thereof, a PE2 system or variant thereof, or a PE3 (e.g. PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### Guide Molecules

The CRISPR-Cas or Cas-Based system described herein can, in some embodiments, include one or more guide molecules. The terms guide molecule, guide sequence and guide polynucleotide, refer to polynucleotides capable of guiding Cas to a target genomic locus and are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net).

A guide sequence, and hence a nucleic acid-targeting guide may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm, and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in PCT US2019/045582, specifically paragraphs [0178]-[0333]. which is incorporated herein by reference.

### Target Sequences, PAMs, and PFSs

### Target Sequences

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table 3 below (from Gleditzsch et al.) shows several Cas polypeptides and the PAM sequence they recognize.

TABLE 3
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including LbCpf1 | TTTV |
| and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a preferred embodiment, the CRISPR effector protein may recognize a 3′ PAM. In certain embodiments, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′ end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Zinc Finger Nucleases

In some embodiments, the target polynucleotide is modified using a Zinc Finger nuclease or system thereof. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP). ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Sequences Related to Nucleus Targeting and Transportation

In some embodiments, one or more components (e.g., the Cas protein and/or deaminase) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID No. 3) or PKKKRKVEAS (SEQ ID No. 4); the NLS from nucleoplasmin (e.g., the nucleoplasmin bipartite NLS with the sequence KRPAATKKAGQAKKKK (SEQ ID No. 5)); the c-myc NLS having the amino acid sequence PAAKRVKLD (SEQ ID No. 6) or RQRRNELKRSP (SEQ ID No. 7); the hRNPA1 M9 NLS having the sequence NQSSNFGPMKGGNFGGRSSGPYGGGGQYFAKPRNQGGY (SEQ ID No. 8); the sequence RMRIZFKNKGKDTAELRRRRVEVSVELRKAKKDEQILKRRNV (SEQ ID No. 9) of the IBB domain from importin-alpha; the sequences VSRKRPRP (SEQ ID No. 10) and PPKKARED (SEQ ID No. 11) of the myoma T protein; the sequence PQPKKKPL (SEQ ID No. 12) of human p53; the sequence SALIKKKKKMAP (SEQ ID No. 13) of mouse c-abl IV; the sequences DRLRR (SEQ ID No. 14) and PKQKKRK (SEQ ID No. 15) of the influenza virus NS1; the sequence RKLKKKIKKL (SEQ ID No. 16) of the Hepatitis virus delta antigen; the sequence REKKKFLKRR (SEQ ID No. 17) of the mouse Mx1 protein; the sequence KRKGDEVDGVDEVAKKKSKK (SEQ ID No. 18) of the human poly(ADP-ribose) polymerase; and the sequence RKCLQAGMNLEARKTKK (SEQ ID No. 19) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein, or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g. aptamers) for adapter proteins, which may be linked to or fused to an nucleotide deaminase or catalytic domain thereof. When such a guide forms a CRISPR complex (e.g., CRISPR-Cas protein binding to guide and target) the adapter proteins bind and, the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g. due to steric hindrance within the three dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### Templates

In some embodiments, the composition for engineering cells comprise a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include sequence that corresponds to both, a first site on the target sequence that is cleaved in a first Cas protein mediated event, and a second site on the target sequence that is cleaved in a second Cas protein mediated event.

In certain embodiments, the template nucleic acid can include sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include sequence which, when integrated, results in: decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include sequence which results in: a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 1 10+/−10, 120+/−10, 130+/−10, 140+/−10, 150+/−10, 160+/−10, 170+/−10, 1 80+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 1 10+/−20, 120+/−20, 130+/−20, 140+/−20, I 50+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149).

### TALE Nucleases

In some embodiments, a TALE nuclease or TALE nuclease system can be used to modify a target polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 20)
MDPIRSRTPSPARELLSGPQPDGVQPTADRGVSPPAGGP
LDGLPARRTMSRTRLPSPPAPSPAFSADSFSDLLRQFDPSLFNTS
LFDSLPPFGAHHTEAATGEWDEVQSGLRAADAPPPTMRVAVTA
ARPPRAKPAPRRRAAQPSDASPAAQVDLRTLGYSQQQQEKIKP
KVRSTVAQHHEALVGHGFTHAHIVALSQHPAALGTVAVKYQD
MIAALPEATHEAIVGVGKQWSGARALEALLTVAGELRGPPLQL
DTGQLLKIAKRGGVTAVEAVHAWRNALTGAPLN

An exemplary amino acid sequence of a C-terminal capping region is:

(SEQ ID NO: 21)
RPALESIVAQLSRPDPALAALTNDHLVALACLGGRPAL
DAVKKGLPHAPALIKRTNRRIPERTSHRVADHAQVVRVLGFFQ
CHSHPAQAFDDAMTQFGMSRHGLLQLFRRVGVTELEARSGTLP
PASQRWDRILQASGMKRAKPSPTSTQTPDQASLHAFADSLERD
LDAPSPMHEGDQTRAS

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

### Meganucleases

In some embodiments, a meganuclease or system thereof can be used to modify a target polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated by reference.

### RNAi

In certain embodiments, the genetic modifying agent is RNAi (e.g., shRNA). As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Diseases

It will be understood by the skilled person that treating as referred to herein encompasses enhancing treatment, or improving treatment efficacy. Treatment may include inhibition of an inflammatory response, enhancing an immune response, tumor regression as well as inhibition of tumor growth, metastasis or tumor cell proliferation, or inhibition or reduction of otherwise deleterious effects associated with the tumor.

Efficaciousness of treatment is determined in association with any known method for diagnosing or treating the particular disease. The invention comprehends a treatment method comprising any one of the methods or uses herein discussed.

The phrase “therapeutically effective amount” as used herein refers to a sufficient amount of a drug, agent, or compound to provide a desired therapeutic effect.

As used herein “patient” refers to any human being receiving or who may receive medical treatment and is used interchangeably herein with the term “subject”.

Therapy or treatment according to the invention may be performed alone or in conjunction with another therapy, and may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the patient, the stage of the cancer, and how the patient responds to the treatment. Additionally, a person having a greater risk of developing an inflammatory response (e.g., a person who is genetically predisposed or predisposed to allergies or a person having a disease characterized by episodes of inflammation) may receive prophylactic treatment to inhibit or delay symptoms of the disease.

The disclosure provides CGRP or derivatives thereof, or an agonist of the CGRP receptor for treating disease. A skilled person can readily determine diseases that can be treated by reducing an ILC2 inflammatory response. ILC2 cells and ILC2 inflammatory responses have been associated with allergic asthma, therapy resistant-asthma, steroid-resistant severe allergic airway inflammation, systemic steroid-dependent severe eosinophilic asthma, chronic rhino-sinusitis (CRS), atopic dermatitis, food allergies, persistence of chronic airway inflammation, and primary eosinophilic gastrointestinal disorders (EGIDs), including but not limited to eosinophilic esophagitis (EoE), eosinophilic gastritis, eosinophilic gastroenteritis, and eosinophilic colitis (see, e.g., Van Rijt et al., Type 2 innate lymphoid cells: at the cross-roads in allergic asthma, Seminars in Immunopathology July 2016, Volume 38, Issue 4, pp 483-496; Rivas et al., IL-4 production by group 2 innate lymphoid cells promotes food allergy by blocking regulatory T-cell function, J Allergy Clin Immunol. 2016 September; 138(3):801-811.e9; and Morita, Hideaki et al. Innate lymphoid cells in allergic and nonallergic inflammation, Journal of Allergy and Clinical Immunology, Volume 138, Issue 5, 1253-1264). Asthma is characterized by recurrent episodes of wheezing, shortness of breath, chest tightness, and coughing. Sputum may be produced from the lung by coughing but is often hard to bring up. During recovery from an attack, it may appear pus-like due to high levels of eosinophils. Symptoms are usually worse at night and in the early morning or in response to exercise or cold air. Some people with asthma rarely experience symptoms, usually in response to triggers, whereas others may have marked and persistent symptoms. CRS is characterized by inflammation of the mucosal surfaces of the nose and para-nasal sinuses, and it often coexists with allergic asthma. Atopic dermatitis is a chronic inflammatory skin disease that is characterized by eosinophilic infiltration and high serum IgE levels. Similar to allergic asthma and CRS, atopic dermatitis has been associated with increased expression of TSLP, IL-25, and IL-33 in the skin. Primary eosinophilic gastrointestinal disorders (EGIDs), including eosinophilic esophagitis (EoE), eosinophilic gastritis, eosinophilic gastroenteritis, and eosinophilic colitisare disorders that exhibit eosinophil-rich inflammation in the gastrointestinal tract in the absence of known causes for eosinophilia such as parasite infection and drug reaction. Not being bound by a theory, corticosteroids suppress TH2 cells, but not ILC2s and cannot be used to modulate ILC2 inflammatory responses. Applicants have discovered factors that balance homeostatic and pathological pro-inflammatory ILC2 responses. In certain embodiments, modulation of these factors, as described herein, may be used to treat the diseases described. In preferred embodiments, CGRP signaling is modulated. In certain embodiments, the treatment can maintain homeostasis of intestinal KLRG^{Hi} ST2⁻ILC2s and prevent their migration to peripheral sites (e.g., lungs) (see, Huang et al., 2017).

In certain embodiments an ILC2 mediated disease or disorder that can be treated by reducing an ILC2 inflammatory response or maintaining ILC2 homeostasis may be any inflammatory disease or disorder such as, but not limited to, asthma, allergy, allergic rhinitis, allergic airway inflammation, atopic dermatitis (AD), chronic obstructive pulmonary disease (COPD), inflammatory bowel disease (IBD), multiple sclerosis, arthritis, psoriasis, eosinophilic esophagitis, eosinophilic pneumonia, eosinophilic psoriasis, hypereosinophilic syndrome, graft-versus-host disease, uveitis, cardiovascular disease, pain, multiple sclerosis, lupus, vasculitis, chronic idiopathic urticaria and Eosinophilic Granulomatosis with Polyangiitis (Churg-Strauss Syndrome).

The asthma may be allergic asthma, non-allergic asthma, severe refractory asthma, asthma exacerbations, viral-induced asthma or viral-induced asthma exacerbations, steroid resistant asthma, steroid sensitive asthma, eosinophilic asthma or non-eosinophilic asthma and other related disorders characterized by airway inflammation or airway hyperresponsiveness (AHR).

The COPD may be a disease or disorder associated in part with, or caused by, cigarette smoke, air pollution, occupational chemicals, allergy or airway hyperresponsiveness.

The allergy may be associated with foods, pollen, mold, dust mites, animals, or animal dander.

The IBD may be ulcerative colitis (UC), Crohn's Disease, collagenous colitis, lymphocytic colitis, ischemic colitis, diversion colitis, Behcet's syndrome, infective colitis, indeterminate colitis, and other disorders characterized by inflammation of the mucosal layer of the large intestine or colon.

The arthritis may be selected from the group consisting of osteoarthritis, rheumatoid arthritis and psoriatic arthritis.

The disclosure also provides methods for enhancing an ILC2 type response and treating disease. In certain embodiments, tissue inflammatory ILC2s are switched to activated, tissue protective ILC2s. ILC2 cells have been shown to promote an eosinophil cytotoxic response, antitumor response and metastasis suppression (Ikutani et al., Identification of Innate IL-5-Producing Cells and Their Role in Lung Eosinophil Regulation and Antitumor Immunity, J Immunol 2012; 188:703-713). Specifically, innate IL-5-producing cells were increased in response to tumor invasion, and their regulation of eosinophils was critical to suppress tumor metastasis. Thus, in one embodiment induction of an ILC2 inflammatory response may be used in treating cancer. In other embodiments, the cancer is resistant to therapies targeting the adaptive immune system (see e.g., Rooney et al., Molecular and genetic properties of tumors associated with local immune cytolytic activity, Cell. 2015 January 15; 160(1-2): 48-61). In one embodiment, modulation of CGRP signaling is used for inducing an inflammatory immune response state for the treatment of a subpopulation of tumor cells that are linked to resistance to targeted therapies and progressive tumor growth. Not being bound by a theory, in cases where tumors are resistant to therapies targeting the adaptive immune system, treatments targeting the innate immune system may be therapeutically effective in treating the tumor.

The cancer may include, without limitation, liquid tumors such as leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, or multiple myeloma.

The cancer may include, without limitation, solid tumors such as sarcomas and carcinomas. Examples of solid tumors include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, epithelial carcinoma, bronchogenic carcinoma, hepatoma, colorectal cancer (e.g., colon cancer, rectal cancer), anal cancer, pancreatic cancer (e.g., pancreatic adenocarcinoma, islet cell carcinoma, neuroendocrine tumors), breast cancer (e.g., ductal carcinoma, lobular carcinoma, inflammatory breast cancer, clear cell carcinoma, mucinous carcinoma), ovarian carcinoma (e.g., ovarian epithelial carcinoma or surface epithelial-stromal tumour including serous tumour, endometrioid tumor and mucinous cystadenocarcinoma, sex-cord-stromal tumor), prostate cancer, liver and bile duct carcinoma (e.g., hepatocelluar carcinoma, cholangiocarcinoma, hemangioma), choriocarcinoma, seminoma, embryonal carcinoma, kidney cancer (e.g., renal cell carcinoma, clear cell carcinoma, Wilm's tumor, nephroblastoma), cervical cancer, uterine cancer (e.g., endometrial adenocarcinoma, uterine papillary serous carcinoma, uterine clear-cell carcinoma, uterine sarcomas and leiomyosarcomas, mixed mullerian tumors), testicular cancer, germ cell tumor, lung cancer (e.g., lung adenocarcinoma, squamous cell carcinoma, large cell carcinoma, bronchioloalveolar carcinoma, non-small-cell carcinoma, small cell carcinoma, mesothelioma), bladder carcinoma, signet ring cell carcinoma, cancer of the head and neck (e.g., squamous cell carcinomas), esophageal carcinoma (e.g., esophageal adenocarcinoma), tumors of the brain (e.g., glioma, glioblastoma, medullablastoma, astrocytoma, medulloblastoma, craniopharyngioma, ependymoma, pinealoma, hemangioblastoma, acoustic neuroma, oligodenroglioma, schwannoma, meningioma), neuroblastoma, retinoblastoma, neuroendocrine tumor, melanoma, cancer of the stomach (e.g., stomach adenocarcinoma, gastrointestinal stromal tumor), or carcinoids. Lymphoproliferative disorders are also considered to be proliferative diseases.

### Administration

In certain embodiments, proteins are administered to a subject in need thereof (e.g., CGRP, antibodies). Delivery of therapeutic proteins can be performed according to any method known in the art (see, e.g., Pisal et al., DELIVERY OF THERAPEUTIC PROTEINS, J Pharm Sci. 2010 June; 99(6): 2557-2575; and Cleland et al., Emerging protein delivery methods, Curr Opin Biotechnol. 2001 April; 12(2):212-9.

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

The medicaments of the invention are prepared in a manner known to those skilled in the art, for example, by means of conventional dissolving, lyophilizing, mixing, granulating or confectioning processes. Methods well known in the art for making formulations are found, for example, in Remington: The Science and Practice of Pharmacy, 20th ed., ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York.

Administration of medicaments of the invention may be by any suitable means that results in a compound concentration that is effective for treating or inhibiting (e.g., by delaying) the development of a disease. The compound is admixed with a suitable carrier substance, e.g., a pharmaceutically acceptable excipient that preserves the therapeutic properties of the compound with which it is administered. One exemplary pharmaceutically acceptable excipient is physiological saline. The suitable carrier substance is generally present in an amount of 1-95% by weight of the total weight of the medicament. The medicament may be provided in a dosage form that is suitable for administration. Thus, the medicament may be in form of, e.g., tablets, capsules, pills, powders, granulates, suspensions, emulsions, solutions, gels including hydrogels, pastes, ointments, creams, plasters, drenches, delivery devices, injectables, implants, sprays, or aerosols.

The agents disclosed herein (e.g., CGRP, CGRP receptor agonists or antagonists) may be used in a pharmaceutical composition when combined with a pharmaceutically acceptable carrier. Such compositions comprise a therapeutically-effective amount of the agent and a pharmaceutically acceptable carrier. Such a composition may also further comprise (in addition to an agent and a carrier) diluents, fillers, salts, buffers, stabilizers, solubilizers, and other materials well known in the art. Compositions comprising the agent can be administered in the form of salts provided the salts are pharmaceutically acceptable. Salts may be prepared using standard procedures known to those skilled in the art of synthetic organic chemistry.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methyl sulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. It will be understood that, as used herein, references to specific agents (e.g., CGRP receptor agonists or antagonists), also include the pharmaceutically acceptable salts thereof.

Methods of administrating the pharmacological compositions, including CGRP, agonists, antagonists, antibodies or fragments thereof, to an individual include, but are not limited to, intradermal, intrathecal, intramuscular, intraperitoneal, intravenous, subcutaneous, intranasal, epidural, by inhalation, and oral routes. In preferred embodiments, CGRP protein is administered intraperitoneally as described in the examples. The compositions can be administered by any convenient route, for example by infusion or bolus injection, by absorption through epithelial or mucocutaneous linings (for example, oral mucosa, rectal and intestinal mucosa, and the like), ocular, and the like and can be administered together with other biologically-active agents. Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment; this may be achieved by, for example, and not by way of limitation, local infusion during surgery, topical application, by injection, by means of a catheter, by means of a suppository, or by means of an implant.

Various delivery systems are known and can be used to administer the pharmacological compositions including, but not limited to, encapsulation in liposomes, microparticles, microcapsules; minicells; polymers; capsules; tablets; and the like. In one embodiment, the agent may be delivered in a vesicle, in particular a liposome. In a liposome, the agent is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,837,028 and 4,737,323. In yet another embodiment, the pharmacological compositions can be delivered in a controlled release system including, but not limited to: a delivery pump (See, for example, Saudek, et al., New Engl. J. Med. 321: 574 (1989) and a semi-permeable polymeric material (See, for example, Howard, et al., J. Neurosurg. 71: 105 (1989)). Additionally, the controlled release system can be placed in proximity of the therapeutic target (e.g., a tumor), thus requiring only a fraction of the systemic dose. See, for example, Goodson, In: Medical Applications of Controlled Release, 1984. (CRC Press, Boca Raton, Fla.).

The amount of the agents (e.g., CGRP, CGRP receptor agonist) which will be effective in the treatment of a particular disorder or condition will depend on the nature of the disorder or condition, and may be determined by standard clinical techniques by those of skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of the agent with which to treat each individual patient. In certain embodiments, the attending physician will administer low doses of the agent and observe the patient's response. Larger doses of the agent may be administered until the optimal therapeutic effect is obtained for the patient, and at that point the dosage is not increased further. In general, the daily dose range lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases. In certain embodiments, suitable dosage ranges for intravenous administration of the agent (e.g., intraperitoneal CGRP administration) are generally about 0.1-500 micrograms (μg) of active compound per kilogram (Kg) body weight, preferably about 0.1-0.5 μg/kg. Suitable dosage ranges for intranasal administration are generally about 0.01 pg/kg body weight to 1 mg/kg body weight. In certain embodiments, a composition containing an agent of the present invention is subcutaneously injected in adult patients with dose ranges of approximately 5 to 5000 μg/human and preferably approximately 5 to 500 μg/human as a single dose. It is desirable to administer this dosage 1 to 3 times daily. Effective doses may be extrapolated from dose-response curves derived from in vitro or animal model test systems. Suppositories generally contain active ingredient in the range of 0.5% to 10% by weight; oral formulations preferably contain 10% to 95% active ingredient. Ultimately the attending physician will decide on the appropriate duration of therapy using compositions of the present invention. Dosage will also vary according to the age, weight and response of the individual patient.

Methods for administering antibodies for therapeutic use is well known to one skilled in the art. In certain embodiments, small particle aerosols of antibodies or fragments thereof may be administered (see e.g., Piazza et al., J. Infect. Dis., Vol. 166, pp. 1422-1424, 1992; and Brown, Aerosol Science and Technology, Vol. 24, pp. 45-56, 1996). In certain embodiments, antibodies (e.g., anti-CGRP receptor or anti-CGRP antibodies) are administered in metered-dose propellant driven aerosols. In preferred embodiments, antibodies are used as agonists to depress inflammatory diseases or allergen-induced asthmatic responses. In certain embodiments, antibodies may be administered in liposomes, i.e., immunoliposomes (see, e.g., Maruyama et al., Biochim. Biophys. Acta, Vol. 1234, pp. 74-80, 1995). In certain embodiments, immunoconjugates, immunoliposomes or immunomicrospheres containing an agent of the present invention is administered by inhalation.

In certain embodiments, antibodies may be topically administered to mucosa, such as the oropharynx, nasal cavity, respiratory tract, gastrointestinal tract, eye such as the conjunctival mucosa, vagina, urogenital mucosa, or for dermal application. In certain embodiments, antibodies are administered to the nasal, bronchial or pulmonary mucosa. In order to obtain optimal delivery of the antibodies to the pulmonary cavity in particular, it may be advantageous to add a surfactant such as a phosphoglyceride, e.g. phosphatidylcholine, and/or a hydrophilic or hydrophobic complex of a positively or negatively charged excipient and a charged antibody of the opposite charge.

Other excipients suitable for pharmaceutical compositions intended for delivery of antibodies to the respiratory tract mucosa may be a) carbohydrates, e.g., monosaccharides such as fructose, galactose, glucose. D-mannose, sorbiose, and the like; disaccharides, such as lactose, trehalose, cellobiose, and the like; cyclodextrins, such as 2-hydroxypropyl-β-cyclodextrin; and polysaccharides, such as raffinose, maltodextrins, dextrans, and the like; b) amino acids, such as glycine, arginine, aspartic acid, glutamic acid, cysteine, lysine and the like; c) organic salts prepared from organic acids and bases, such as sodium citrate, sodium ascorbate, magnesium gluconate, sodium gluconate, tromethamine hydrochloride, and the like: d) peptides and proteins, such as aspartame, human serum albumin, gelatin, and the like; e) alditols, such mannitol, xylitol, and the like, and f) polycationic polymers, such as chitosan or a chitosan salt or derivative.

For dermal application, the antibodies of the present invention may suitably be formulated with one or more of the following excipients: solvents, buffering agents, preservatives, humectants, chelating agents, antioxidants, stabilizers, emulsifying agents, suspending agents, gel-forming agents, ointment bases, penetration enhancers, and skin protective agents.

Examples of solvents are water, alcohols, vegetable or marine oils (e.g. edible oils like almond oil, castor oil, cacao butter, coconut oil, corn oil, cottonseed oil, linseed oil, olive oil, palm oil, peanut oil, poppy seed oil, rapeseed oil, sesame oil, soybean oil, sunflower oil, and tea seed oil), mineral oils, fatty oils, liquid paraffin, polyethylene glycols, propylene glycols, glycerol, liquid polyalkylsiloxanes, and mixtures thereof.

Examples of buffering agents are citric acid, acetic acid, tartaric acid, lactic acid, hydrogenphosphoric acid, diethyl amine etc. Suitable examples of preservatives for use in compositions are parabenes, such as methyl, ethyl, propyl p-hydroxybenzoate, butylparaben, isobutylparaben, isopropylparaben, potassium sorbate, sorbic acid, benzoic acid, methyl benzoate, phenoxyethanol, bronopol, bronidox, MDM hydantoin, iodopropynyl butylcarbamate, EDTA, benzalconium chloride, and benzylalcohol, or mixtures of preservatives.

Examples of humectants are glycerin, propylene glycol, sorbitol, lactic acid, urea, and mixtures thereof.

Examples of antioxidants are butylated hydroxy anisole (BHA), ascorbic acid and derivatives thereof, tocopherol and derivatives thereof, cysteine, and mixtures thereof.

Examples of emulsifying agents are naturally occurring gums, e.g., gum acacia or gum tragacanth; naturally occurring phosphatides, e.g., soybean lecithin, sorbitan monooleate derivatives: wool fats; wool alcohols; sorbitan esters; monoglycerides; fatty alcohols; fatty acid esters (e.g,. triglycerides of fatty acids); and mixtures thereof.

Examples of suspending agents are celluloses and cellulose derivatives such as, e.g., carboxymethyl cellulose, hydroxyethylcellulose, hydroxypropylcellulose, hydroxypropylmethylcellulose, carraghenan, acacia gum, arabic gum, tragacanth, and mixtures thereof.

Examples of gel bases, viscosity-increasing agents or components which are able to take up exudate from a wound are liquid paraffin, polyethylene, fatty oils, colloidal silica or aluminum, zinc soaps, glycerol, propylene glycol, tragacanth, carboxyvinyl polymers, magnesium-aluminum silicates, Carbopol®, hydrophilic polymers such as, e.g. starch or cellulose derivatives such as, e.g., carboxymethylcellulose, hydroxyethylcellulose and other cellulose derivatives, water-swellable hydrocolloids, carragenans, hyaluronates (e.g., hyaluronate gel optionally containing sodium chloride), and alginates including propylene glycol alginate.

Examples of ointment bases are e.g. beeswax, paraffin, cetanol, cetyl palmitate, vegetable oils, sorbitan esters of fatty acids (Span), polyethylene glycols, and condensation products between sorbitan esters of fatty acids and ethylene oxide, e.g. polyoxyethylene sorbitan monooleate (Tween).

Examples of hydrophobic or water-emulsifying ointment bases are paraffins, vegetable oils, animal fats, synthetic glycerides, waxes, lanolin, and liquid polyalkylsiloxanes. Examples of hydrophilic ointment bases are solid macrogols (polyethylene glycols). Other examples of ointment bases are triethanolamine soaps, sulphated fatty alcohol and polysorbates.

Examples of other excipients are polymers such as carmelose, sodium carmelose, hydroxypropylmethylcellulose, hydroxyethylcellulose, hydroxypropylcellulose, pectin, xanthan gum, locust bean gum, acacia gum, gelatin, carbomer, emulsifiers like vitamin E, glyceryl stearates, cetanyl glucoside, collagen, carrageenan, hyaluronates and alginates and chitosans.

The dose of antibody required in humans to be effective in the treatment or prevention of allergic inflammation differs with the type and severity of the allergic condition to be treated, the type of allergen, the age and condition of the patient, etc. Typical doses of antibody to be administered are in the range of 1 μg to 1 g, preferably 1-1000 more preferably 2-500, even more preferably 5-50, most preferably 10-20 μg per unit dosage form. In certain embodiments, infusion of antibodies of the present invention may range from 10-500 mg/m².

The one or more therapeutic molecules may be expressed from one or more polynucleotide sequences on one or more vectors (e.g., CGRP, genetic modifying agent). The invention comprehends such polynucleotide molecule(s), for instance such polynucleotide molecules operably configured to express the protein and/or the nucleic acid component(s), as well as such vector(s). Regulatory elements may comprise inducible promotors. Polynucleotides and/or vector systems may comprise inducible systems. For example, the expression of the polynucleotides may be regulated by a tetracycline/doxycycline controlled inducible promoter. In certain embodiments, the vectors are tissue specific. The vector may include a tissue specific regulatory element or be a tissue specific vector (e.g., viral vector).

In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Vectors for and that result in expression in a eukaryotic cell can be referred to herein as “eukaryotic expression vectors.” Common expression vectors of utility in recombinant DNA techniques are often in the form of plasmids. The vectors used herein may include viral vectors or plasmids. In preferred embodiments, viral vectors are used. In more preferred embodiments, lentiviral vectors are used.

In certain embodiments, the vectors used can include a detectable or selectable marker used to select for cells that were transfected or transduced. Selection can use FACS or any cell sorting method. Cells can be selected for by use of a drug resistance marker. In certain embodiments, the detectable marker is a fluorescent protein such as green fluorescent protein (GFP), enhanced green fluorescent protein (EGFP), red fluorescent protein (RFP), blue fluorescent protein (BFP), cyan fluorescent protein (CFP), yellow fluorescent protein (YFP), miRFP (e.g., miRFP670, see, e.g., Shcherbakova, et al., Nat Commun. 2016; 7: 12405), mCherry, tdTomato, DsRed-Monomer, DsRed-Express, DSRed-Express2, DsRed2, AsRed2, mStrawberry, mPlum, mRaspberry, HcRedl, E2-Crimson, mOrange, mOrange2, mBanana, ZsYellowl, TagBFP, mTagBFP2, Azurite, EBFP2, mKalamal, Sirius, Sapphire, T-Sapphire, ECFP, Cerulean, SCFP3A, mTurquoise, mTurquoise2, monomelic Midoriishi-Cyan, TagCFP, niTFPl, Emerald, Superfolder GFP, Monomeric Azami Green, TagGFP2, mUKG, mWasabi, Clover, mNeonGreen, Citrine, Venus, SYFP2, TagYFP, Monomeric Kusabira-Orange, mKOk, mK02, mTangerine, mApple, mRuby, mRuby2, HcRed-Tandem, mKate2, mNeptune, NiFP, mkeima Red, LSS-mKatel, LSS-mKate2, mBeRFP, PA-GFP, PAmCherryl, PATagRFP, TagRFP6457, IFP1.2, iRFP, Kaede (green), Kaede (red), KikGRl (green), KikGRl (red), PS-CFP2, mEos2 (green), mEos2 (red), mEos3.2 (green), mEos3.2 (red), PSmOrange, Dronpa, Dendra2, Timer, AmCyanl, or a combination thereof. In certain embodiments, the detectable marker is a cell surface marker. In other instances, the cell surface marker is a marker not normally expressed on the cells, such as a truncated nerve growth factor receptor (tNGFR), a truncated epidermal growth factor receptor (tEGFR), CD8, truncated CD8, CD19, truncated CD19, a variant thereof, a fragment thereof, a derivative thereof, or a combination thereof. Selectable markers are known in the art and enable selecting for cells having the barcode integrated. Examples of selectable markers include, but are not limited to, antibiotic resistance genes, such as beta-lactamase, neo, FabI, URA3, cam, tet, blasticidin, hyg, puromycin and the like. A selectable marker useful in accordance with the invention may be any selectable marker appropriate for use in a eukaryotic cell, such as a mammalian cell, or more specifically a human cell. One of skill in the art will understand and be able to identify and use selectable markers in accordance with the invention.

The invention also provides a delivery system comprising one or more vectors or one or more polynucleotide molecules, the one or more vectors or polynucleotide molecules comprising one or more polynucleotide molecules encoding components of a non-naturally occurring or engineered composition which is a composition having the characteristics as discussed herein or defined in any of the herein described methods.

The invention also provides a non-naturally occurring or engineered composition, or one or more polynucleotides encoding components of said composition, or delivery systems comprising one or more polynucleotides encoding components of said composition for use in a therapeutic method of treatment. The therapeutic method of treatment may comprise gene or genome editing, or gene therapy.

Delivery vehicles, vectors, particles, nanoparticles, formulations and components thereof for expression of one or more elements of a nucleic acid-targeting system are as used in the foregoing documents, such as WO 2014/093622 (PCT/US2013/074667). In some embodiments, a vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

The invention also provides an expression vector comprising any of the above-described polynucleotide molecules. The invention also provides such polynucleotide molecule(s), for instance such polynucleotide molecules operably configured to express the protein and/or the nucleic acid component(s), as well as such vector(s).

In practicing any of the methods disclosed herein, a suitable vector can be introduced to a cell or an embryo via one or more methods known in the art, including without limitation, microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In some methods, the vector is introduced into an embryo by microinjection. The vector or vectors may be microinjected into the nucleus or the cytoplasm of the embryo. In some methods, the vector or vectors may be introduced into a cell by nucleofection.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection.

In another aspect, provided is a pharmaceutical pack or kit, comprising one or more containers filled with one or more of the ingredients of the pharmaceutical compositions and CGRP receptor agonists or antagonists (e.g., CGRP).

### Diagnostic and Screening Methods

The invention provides biomarkers (e.g., phenotype specific or cell type) for the identification, diagnosis, prognosis and manipulation of cell properties, for use in a variety of diagnostic and/or therapeutic indications. Biomarkers in the context of the present invention encompasses, without limitation nucleic acids, proteins, reaction products, and metabolites, together with their polymorphisms, mutations, variants, modifications, subunits, fragments, and other analytes or sample-derived measures. In certain embodiments, biomarkers include the signature genes or signature gene products, and/or cells as described herein.

Biomarkers are useful in methods of diagnosing, prognosing and/or staging an immune response in a subject by detecting a first level of expression, activity and/or function of one or more biomarker and comparing the detected level to a control of level wherein a difference in the detected level and the control level indicates that the presence of an immune response in the subject.

The terms “diagnosis” and “monitoring” are commonplace and well-understood in medical practice. By means of further explanation and without limitation the term “diagnosis” generally refers to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

The biomarkers of the present invention are useful in methods of identifying patient populations at risk or suffering from an immune response based on a detected level of expression, activity and/or function of one or more biomarkers. These biomarkers are also useful in monitoring subjects undergoing treatments and therapies for suitable or aberrant response(s) to determine efficaciousness of the treatment or therapy and for selecting or modifying therapies and treatments that would be efficacious in treating, delaying the progression of or otherwise ameliorating a symptom. The biomarkers provided herein are useful for selecting a group of patients at a specific state of a disease with accuracy that facilitates selection of treatments.

The term “monitoring” generally refers to the follow-up of a disease or a condition in a subject for any changes which may occur over time.

The terms also encompass prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-á-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-á-vis a control subject or subject population.

Suitably, an altered quantity or phenotype of the immune cells in the subject compared to a control subject having normal immune status or not having a disease comprising an immune component indicates that the subject has an impaired immune status or has a disease comprising an immune component or would benefit from an immune therapy.

Hence, the methods may rely on comparing the quantity of immune cell populations, biomarkers, or gene or gene product signatures measured in samples from patients with reference values, wherein said reference values represent known predictions, diagnoses and/or prognoses of diseases or conditions as taught herein.

For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

In a further example, distinct reference values can represent the diagnosis of a given disease or condition as taught herein vs. the diagnosis of no such disease or condition (such as, e.g., the diagnosis of healthy, or recovered from said disease or condition, etc.). In another example, distinct reference values may represent the diagnosis of such disease or condition of varying severity.

In yet another example, distinct reference values may represent a good prognosis for a given disease or condition as taught herein vs. a poor prognosis for said disease or condition. In a further example, distinct reference values may represent varyingly favourable or unfavourable prognoses for such disease or condition.

Such comparison may generally include any means to determine the presence or absence of at least one difference and optionally of the size of such difference between values being compared. A comparison may include a visual inspection, an arithmetical or statistical comparison of measurements. Such statistical comparisons include, but are not limited to, applying a rule.

Reference values may be established according to known procedures previously employed for other cell populations, biomarkers and gene or gene product signatures. For example, a reference value may be established in an individual or a population of individuals characterised by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR-), Youden index, or similar.

In one embodiment, the signature genes, biomarkers, and/or cells may be detected or isolated by immunofluorescence, immunohistochemistry (IHC), fluorescence activated cell sorting (FACS), mass spectrometry (MS), mass cytometry (CyTOF), RNA-seq, single cell RNA-seq (described further herein), quantitative RT-PCR, single cell qPCR, FISH, RNA-FISH, MERFISH (multiplex (in situ) RNA FISH) and/or by in situ hybridization. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25).

In certain embodiments, diseases related to ILC2 responses as described further herein are diagnosed, prognosed, or monitored. For example, a tissue sample may be obtained and analyzed for specific cell markers (IHC) or specific transcripts (e.g., RNA-FISH). Tissue samples for diagnosis, prognosis or detecting may be obtained by endoscopy. In one embodiment, a sample may be obtained by endoscopy and analyzed b FACS. As used herein, “endoscopy” refers to a procedure that uses an endoscope to examine the interior of a hollow organ or cavity of the body. The endoscope may include a camera and a light source. The endoscope may include tools for dissection or for obtaining a biological sample. A cutting tool can be attached to the end of the endoscope, and the apparatus can then be used to perform surgery. Applications of endoscopy that can be used with the present invention include, but are not limited to examination of the oesophagus, stomach and duodenum (esophagogastroduodenoscopy); small intestine (enteroscopy); large intestine/colon (colonoscopy, sigmoidoscopy); bile duct; rectum (rectoscopy) and anus (anoscopy), both also referred to as (proctoscopy); respiratory tract; nose (rhinoscopy); lower respiratory tract (bronchoscopy); ear (otoscope); urinary tract (cystoscopy); female reproductive system (gynoscopy); cervix (colposcopy); uterus (hysteroscopy); fallopian tubes (falloposcopy); normally closed body cavities (through a small incision); abdominal or pelvic cavity (laparoscopy); interior of a joint (arthroscopy); or organs of the chest (thoracoscopy and mediastinoscopy).

In certain embodiments, the method provides for treating a patient with CGRP, wherein the patient is suffering from a disease related to ILC2 inflammatory responses (e.g., allergy), the method comprising the steps of determining whether the patient expresses a gene signature, biological program or marker gene as described herein; obtaining or having obtained a biological sample from the patient; and performing or having performed an assay as described herein on the biological sample to determine if the patient expresses the gene signature, biological program or marker gene; and if the patient has an ILC2 inflammatory gene signature, biological program or marker gene, then administering CGRP to the patient in an amount sufficient to shift the phenotype to a homeostatic phenotype, and if the patient does not have an ILC2 inflammatory gene signature, biological program or marker gene, then not administering CGRP to the patient, wherein a risk of having inflammatory symptoms is increased if the patient has an ILC2 inflammatory gene signature, biological program or marker gene.

The present invention also may comprise a kit with a detection reagent that binds to one or more biomarkers or can be used to detect one or more biomarkers.

### Methods of Quantitating a Type 2 Immune Response

In certain embodiments, a method of quantitating a type 2 immune response comprises determining the ILC2 frequency, wherein increased frequency of ILC2s as compared to a control frequency is associated with an increased type 2 immune response. As used herein, “frequency” refers to the rate of cells in a given sample. For example, frequency=the number of a cell type divided by the total number of all cell types in a sample. In certain embodiments, flow cytometry techniques are used to determine the frequency (e.g., FACS). The frequency may be in relation to all cells, all immune cells or all ILC cells in a population of cells obtained from a subject. In certain embodiments, the method further comprises determining the frequency of one or more cells selected from the group consisting of: mast cells, macrophages, neutrophils, and CD11b+CD103+ dendritic cells, wherein increased frequency of mast cells, macrophages and/or neutrophils, and/or decreased frequency of CD11b+CD103+ dendritic cells as compared to a control frequency is associated with an increased type 2 immune response. The frequency may be in relation to all cells, all immune cells or all ILC cells in a population of cells obtained from a subject. In certain embodiments, the method is a diagnostic method for determining an immune response in a subject in need thereof. In certain embodiments, a subject is monitored during treatment of an aberrant immune response.

In certain embodiments, a method of quantitating a type 2 immune response comprises determining the expression of one or more genes selected from Table 3 or determining the frequency of the cell types expressing the one or more genes selected from Table 3, wherein changes in expression or frequency according to Table 3 is associated with an increased type 2 immune response.

In certain embodiments, the method comprises determining expression of one or more genes selected from Srgn, Hes1, Sla, Ppp1r15a, Furin, Actg1, Il13, Hspa8, Lilr4b, 4930523C07Rik and Dnaja1 in LP cluster 12.

In certain embodiments, the method comprises determining expression of one or more genes selected from Sla, Jund, Klf4 and Dusp1 in LP cluster 15.

In certain embodiments, the method comprises determining expression of one or more genes selected from Txnip, Mcpt1, Igkv1-135, Mcpt2, Ighg1, Igkv12-44, Ifi2712a, Ubald2, Cacna1s, Sepp1, Pdia6, Rilpl2, Iglc2, Dusp5, Fosb, Serp1, Grasp, Ccr10, Ddit4, Malat1, Pim1, Hsp90b1, Trf, Ifi27, Odd and Xbp1 in LP cluster 20.

In certain embodiments, the method comprises determining expression of one or more genes selected from Btg2, Junb, Ubb, Dusp1, Bcl2, Pnrc1, Pim1, Jund, Actg1, Btg1 and Irf7 in LP cluster 3.

In certain embodiments, the method comprises determining expression of one or more genes selected from Jun, Zfp3612, Fos, Neat1 and Irf7 in LP cluster 6.

In certain embodiments, the method comprises determining expression of one or more genes selected from Tsc22d3, Wdr89, Txnip, Uba52, Ddit4, Bcl2, Cd74 and Jund in PP cluster 1.

In certain embodiments, the method comprises determining expression of one or more genes selected from Zfp3612, Tsc22d3 in PP cluster 2.

In certain embodiments, the method comprises determining expression of one or more genes selected from Jund, Id2, Pim1, Nfkbia, Klf4, Tgif1, Hk2, Junb, Gimap1 and Dusp5 in PP cluster 3.

In certain embodiments, the method comprises determining expression of one or more genes selected from Jund, Klf4, Junb, Lmna, Ncoa7, Dusp1 and Pim1 in PP cluster 4.

In certain embodiments, the method comprises determining expression of one or more genes selected from Tsc22d3 in PP cluster 5.

In certain embodiments, the method comprises determining expression of one or more genes selected from Mcpt1 and Defa24 in PP cluster 6.

In certain embodiments, the method comprises determining expression of one or more genes selected from Jund in PP cluster 8.

In certain embodiments, the method comprises determining expression of one or more genes selected from Igha in PP cluster 9.

### MS Methods

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III

TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

### Immunoassays

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Hybridization Assays

Such applications are hybridization assays in which a nucleic acid that displays “probe” nucleic acids for each of the genes to be assayed/profiled in the profile to be generated is employed.

In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992, the disclosures of which are herein incorporated by reference, as well as International Patent Publication Nos. WO 95/21265, WO 96/31622, WO 97/10365, and WO 97/27317; European Patent Application Nos. EP 373203; and EP 785280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65 C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif. (1992).

### Sequencing and Single Cell Sequencing

In certain embodiments, the invention involves targeted nucleic acid profiling (e.g., sequencing, quantitative reverse transcription polymerase chain reaction, and the like) (see e.g., Geiss G K, et al., Direct multiplexed measurement of gene expression with color-coded probe pairs. Nat Biotechnol. 2008 March; 26(3):317-25). In certain embodiments, a target nucleic acid molecule (e.g., RNA molecule), may be sequenced by any method known in the art, for example, methods of high-throughput sequencing, also known as next generation sequencing or deep sequencing. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others.

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

In certain embodiments, the invention involves the Assay for Transposase Accessible Chromatin using sequencing (ATAC-seq) as described. (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US Patent Publication Nos. US 2016-0208323 A1 and US 2016-0060691A1; and International Patent Publication No. WO 2017/156336A1).

### Methods of Screening

A further aspect of the invention relates to a method for identifying an agent capable of modulating one or more phenotypic aspects of a cell or cell population as disclosed herein, comprising: a) applying a candidate agent to the cell or cell population; b) detecting modulation of one or more phenotypic aspects of the cell or cell population by the candidate agent, thereby identifying the agent. The phenotypic aspects of the cell or cell population that is modulated may be a gene signature or biological program specific to a cell type or cell phenotype or phenotype specific to a population of cells (e.g., an ILC2 immune response phenotype). In certain embodiments, steps can include administering candidate modulating agents to cells, detecting identified cell (sub)populations for changes in signatures, or identifying relative changes in cell (sub) populations which may comprise detecting relative abundance of particular gene signatures.

The term “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of an immune cell or immune cell population may be modulated without substantially altering other (unintended, undesired) phenotypic aspect(s).

The term “agent” broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place.

Agents may include any potential class of biologically active conditions, substances or agents, such as for instance antibodies, proteins, peptides, nucleic acids, oligonucleotides, small molecules, or combinations thereof, as described herein.

The methods of phenotypic analysis can be utilized for evaluating environmental stress and/or state, for screening of chemical libraries, and to screen or identify structural, synthetic, genomic, and/or organism and species variations. For example, a culture of cells, can be exposed to an environmental stress, such as but not limited to heat shock, osmolarity, hypoxia, cold, oxidative stress, radiation, starvation, a chemical (for example a therapeutic agent or potential therapeutic agent) and the like. After the stress is applied, a representative sample can be subjected to analysis, for example at various time points, and compared to a control, such as a sample from an organism or cell, for example a cell from an organism, or a standard value. By exposing cells, or fractions thereof, tissues, or even whole animals, to different members of the chemical libraries, and performing the methods described herein, different members of a chemical library can be screened for their effect on immune phenotypes thereof simultaneously in a relatively short amount of time, for example using a high throughput method.

Aspects of the present disclosure relate to the correlation of an agent with the spatial proximity and/or epigenetic profile of the nucleic acids in a sample of cells. In some embodiments, the disclosed methods can be used to screen chemical libraries for agents that modulate chromatin architecture epigenetic profiles, and/or relationships thereof.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

In certain embodiments, the present invention provides for gene signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The signatures or biological programs of the present invention may be used to screen for drugs that reduce the signature or biological program in cells as described herein. The signature or biological program may be used for GE-HTS. In certain embodiments, pharmacological screens may be used to identify drugs that are selectively toxic to cells having a signature.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). In certain embodiments, Cmap can be used to screen for small molecules capable of modulating a signature or biological program of the present invention in silico.

The invention is further described in the following examples, which do not limit the scope of the invention described in the claims.

### EXAMPLES

### Example 1—an Atlas of Mouse Small Intestinal Immune Cells at Homeostasis and in Type 2 Inflammation

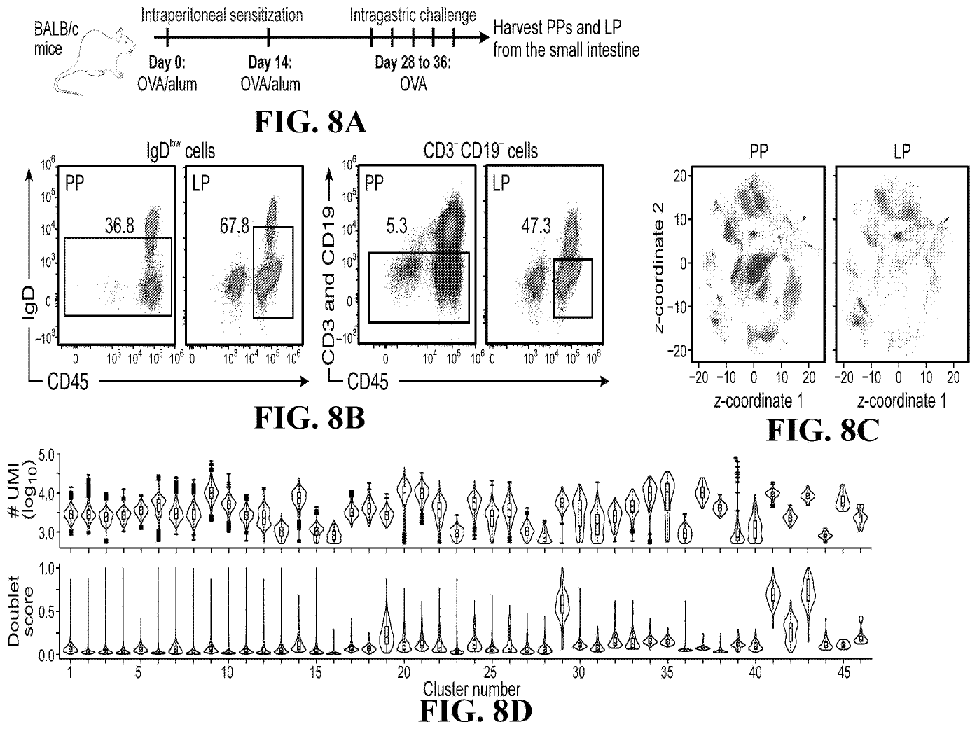

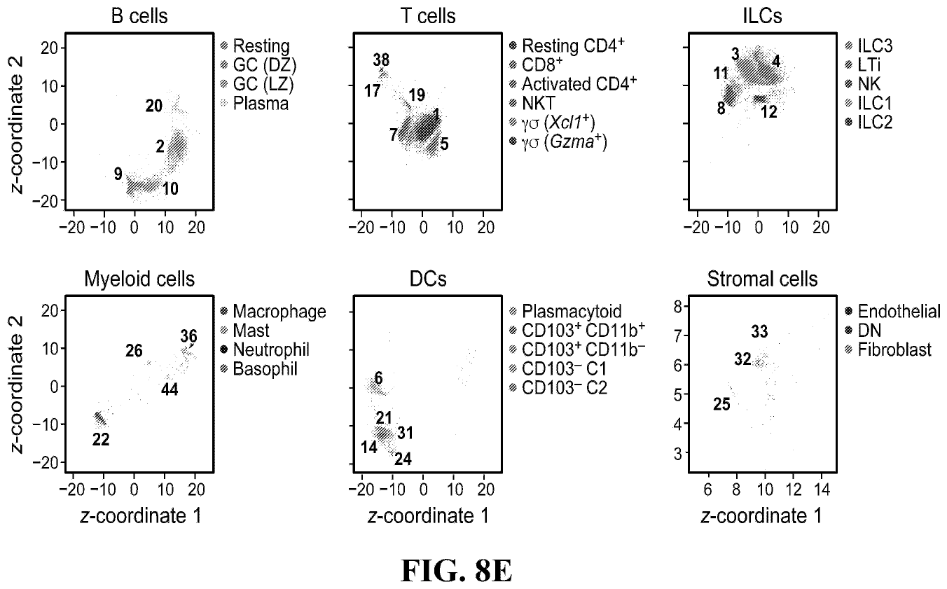

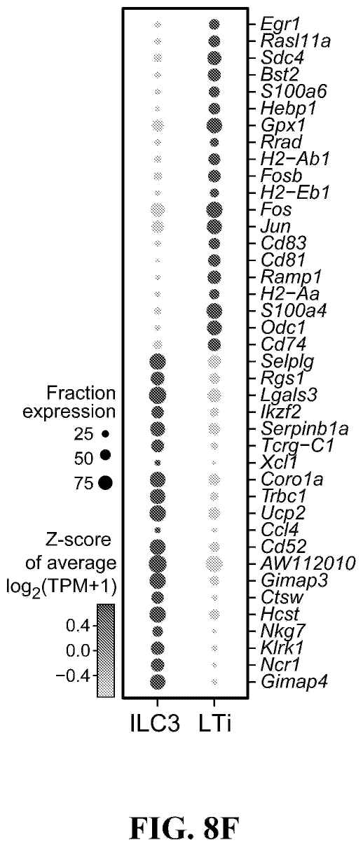

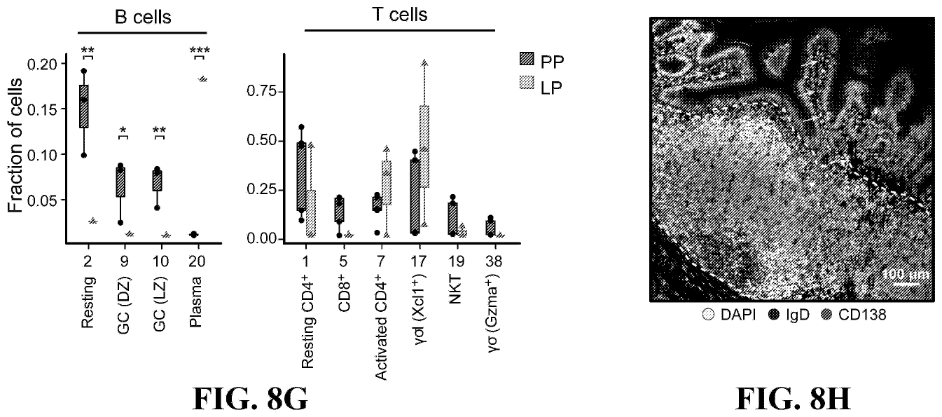

To comprehensively characterize small intestinal immune cells, Applicants generated scRNA-Seq profiles for 58,067 immune cells collected from lamina propria (LP) and Peyer's patch (PP) regions at steady state and after induction of a type 2 inflammatory reaction to ovalbumin (OVA) (Brandt et al., 2003) (FIGS. 1A and 8A). To ensure sufficient representation of rare cells, Applicants combined a normalization strategy—removing by flow sorting either a portion of IgD+ naïve B cells, which are the most abundant cell type in PPs, or all CD19+ and CD3+ T cells (FIG. 8B, Methods)—with profiling of a sufficiently large number of cells number (P>0.99 to recover at least 15 cells from each cluster, Methods) by massively parallel scRNA-seq (Haber et al., 2017) (Methods). Applicants obtained 36,797 and 21,270 high quality cells passing initial filtering (Methods) from PP and LP regions, respectively.

Overall, Applicants annotated 27 cell subsets across the two compartments, spanning T cells, B cells, ILCs, dendritic cells (DCs), myeloid cells and stromal cells, in frequencies ranging from 0.07% to 14%. Briefly, Applicants first partitioned the cells from the PP regions by unsupervised clustering (Methods) into 46 clusters, retained the 97% of cells that were robustly assigned cell identities in each cluster (Methods), assigned the cells from the LP regions to the 46 clusters using a classifier (Methods), and visualized the data with scvis (Ding et al., 2018), a deep generative model-based method (FIGS. 1B and 8C, Methods). Next, Applicants identified genes that were differentially expressed between the cells in each cluster and the remaining cells (Table 1, Methods), as well as between pairs of clusters (Table 2), to elucidate identities of similar cell types/states. Applicants excluded from downstream analysis clusters mostly consisting of contaminants from epithelium, likely doublets, or cells with a small number of Unique Molecular Identifiers (UMIs) (FIGS. 1C and 8D), or lacking distinct markers (Methods). Altogether, Applicants retained 27 clusters spanning 85% of the cells and each associated with distinct marker genes (FIG. 8E, Methods).

Importantly, known distinct cell types with highly similar expression profiles were correctly distinguished by the analysis. For example, NKp46+ILC3s and CCR6+ lymphoid tissue inducer (LTi) cells were recovered as separate clusters, even though they both require RORγt controlled expression programs for their fate determination (Robinette et al., 2015). Nevertheless, LTi cells were distinguished by significantly higher expression levels of MHCII antigen presentation modules (FIG. 8F, adjusted P<0.05, likelihood ratio test), whose expression on ILCs has been shown to regulate intestinal homeostasis (Hepworth et al., 2013).

The atlas highlighted expected differences in cellular composition between the LP and PP regions (FIGS. 1D and 8G). For example, resting and IgD⁻GL7⁺ germinal center (GC) B cells were exclusively found in PPs (FIG. 8G). Conversely, although CD138⁺ antibody producing plasma cells differentiate from GC B cells, they were predominantly found in LP samples (FIG. 8G) and confirmed in LP regions in situ (FIG. 8H), highlighting their potential requirement for antibody secretion adjacent to microbes and other antigens in the intestinal mucosa. In another example, LTi cells were significantly enriched (adjusted P=4.75*10⁻³, Wald test) in PP vs. LP regions (FIG. 1D), which Applicants confirmed by flow cytometry (FIG. 1E, P<0.001, Fisher's exact test). The LTi cell distribution is consistent with their critical function in supporting lymphoid tissue development. ILC1s and Natural Killer (NK) cells were also significantly enriched in PP regions (FIG. 1D, adjusted P=4.29*10⁻⁷ and P=1.19*10⁻¹⁷, respectively, Wald test), suggesting they have unique functions in PPs. Indeed, NK cells suppress affinity maturation of GC B cells (Rydyznski et al., 2018), which probably requires their localization in B cell follicles.

### Example 2—Increase in ILC2 Proportions and Changes in their Expression Programs are Prominent Features of OVA-Induced Type 2 Inflammation

Cell composition was remodeled in type 2 inflammation (FIGS. 2A, B and 9A), highlighting both known and unique responses. Mast cell expansion, a well-established feature of allergic reactions (Brandt et al., 2003), was observed in OVA-treated mice (adjusted P=1.03*10⁻¹¹, Wald test, FIG. 2B) and validated by flow cytometry (FIG. 2C, P=0.005, t-test). Applicants also observed a significant increase in ILC2 frequency in mice with type 2 immune reactions to OVA (FIG. 2B, P=1.22*10⁻², Wald test), which Applicants validated by flow cytometry (FIG. 2D, P=0.009, t-test), highlighting their importance in this allergic inflammatory model (Burton et al., 2018). Macrophage and neutrophil frequencies also increased significantly in OVA-treated mice (P=3.87*10⁻³ and P=4.15*10⁻³, respectively, Wald test), reflecting immune cell infiltration. CD11b⁺CD103⁺ DCs, one of four different populations of DCs in the intestine (Mowat and Agace, 2014), were the most significantly reduced by OVA-induced inflammation (P=6.04*10⁻⁸, Wald test), suggesting that they were likely involved in the maintenance of gut homeostasis, and suppressed in the type 2 inflammatory condition.

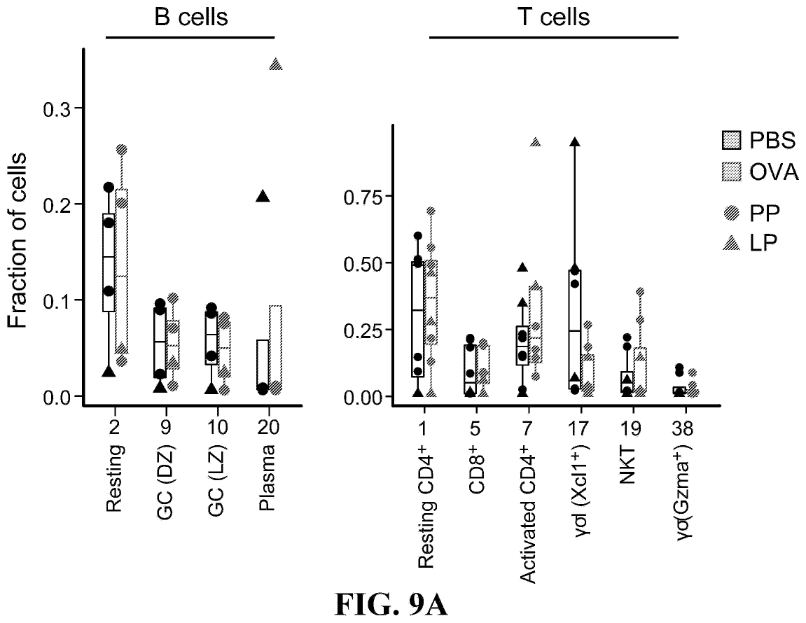

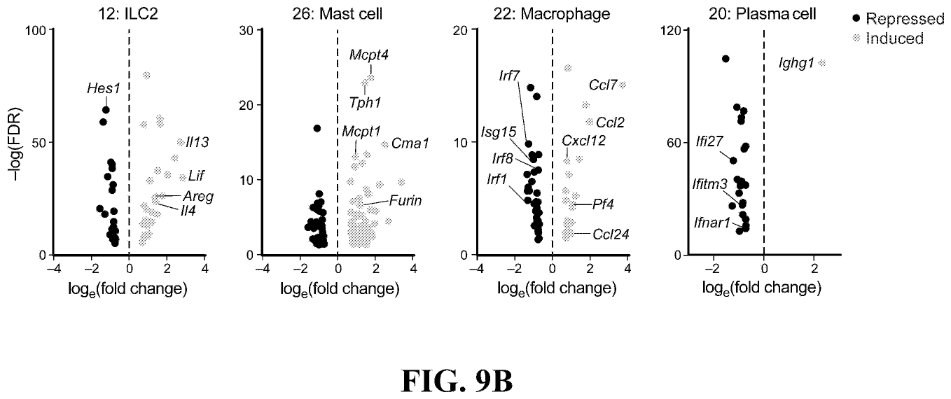

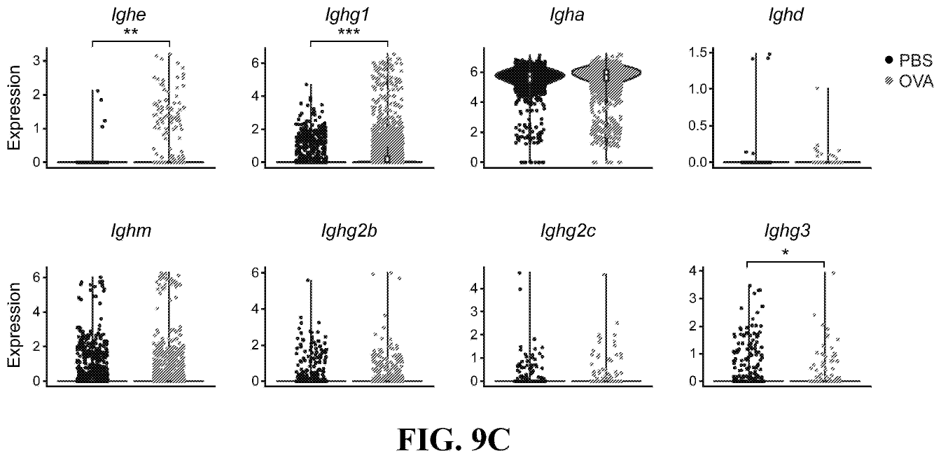

The impact of type 2 inflammation on cell intrinsic expression changes was primarily centered in mast cells and ILC2s in both LP and PP regions (FIG. 2E and Table 3). Genes associated with cell type-specific activation were induced in mast cells (e.g., Mcpt1, Mcpt4) and ILC2s (e.g., Il13, Il4) (FIG. 9B), suggesting an activation phenotype in both cell types. In addition, Ighg1 and Ighe expression in plasma cells was significantly increased (P=1.45*10⁻¹⁰⁷ and 2.86*10⁻¹³, respectively; likelihood ratio test) after the induction of allergic inflammation (FIG. 9C), consistent with the fact that IL-4 induces class-switch recombination of immunoglobulin heavy (Ig) chain towards IgG1 and IgE (Manis et al., 2002). Additionally, genes involved in the type 1 interferon pathway, such as Irf1 and Isg15 in macrophages and Ifnar1 and Ifitm3 in plasma cells, were suppressed during type 2 inflammation (FIG. 9B), providing potential mechanistic explanations for the dampened antiviral immunity in allergic inflammation (Kloepfer et al., 2012; Rowe and Gill, 2015).

### Example 3—Topic Models Identify Cell Type-Specific Programs that are Increased with Inflammation in Mast Cells, ILC2s, Th2 Cells and Fibroblast Subsets

To identify more nuanced changes in expression programs in response to type 2 inflammation, Applicants relied on topic modeling using Latent Dirichlet Allocations (Blei et al., 2003), which has recently been applied to scRNA-seq data (Bielecki et al., 2018; duVerle et al., 2016). Originally developed to discover key semantic topics reflected by the words used in a corpus of documents (Dumais et al., 1990), topic modeling can be used to explore gene programs (“topics”) in each cell (“document”) based on the distribution of genes (“words”) expressed in the cell. A gene can belong to multiple programs, and its relative relevance in the topic is reflected by a weight. A cell is then represented as a weighted mixture of topics, where the weights reflect the importance of the corresponding gene program in the cell. Applicants learned topic models for each cell lineage or group separately, and then searched for topics that were differentially weighted between homeostasis and inflammation in cells of the same type (Methods, Table 4, FIGS. 3A-E and 10A-F).

Topics specifically characterizing activation in mast cells and ILC2s increased in prominence following inflammation. Topic 1 (“mast cell activation program”), which was heavily weighted in the mast cell cluster among myeloid cells, was characterized by Cpa3 and Mcpt4 expression and significantly more prominent in mast cells from mice treated with OVA than controls (adjusted P=2.84*10⁻⁶⁷, Mann-Whitney U test, FIG. 3A). Topic 2 (“ILC2 activation program”), which was heavily weighted in the ILC2 cluster, included type 2 genes such as Il13 and Il4. Topic 2 was significantly more prominent in ILC2s after induction of inflammation (adjusted P=8.01*10⁻¹², Mann-Whitney U test, FIG. 3B). Consistent with the differential expression analysis, these two topics captured the major events induced by inflammation.

Programs related to inflammatory responses were also more heavily weighted in Th2 cells under inflammatory conditions, whereas those related to T regulatory cells were unchanged between inflammation and homeostasis. Specifically, topic 5 (“Th2 inflammatory program”), consisting of type 2 inflammatory genes, including Il13, Gata3, 116, Areg and Il1rl1, was present only in a subset of cells from the activated CD4⁺ T cell cluster that were likely type 2 T helper (TH2) cells (FIG. 3C). Topic 5 weights were significantly higher in cells from OVAtreated mice than from controls (adjusted P=2.73*10⁻⁵, Mann-Whitney U test, FIG. 3C). Topic 9 (“T regulatory program”), comprised of Foxp3, Lag3, Il10 and other Th regulatory genes, was present in a different subset of CD4⁺ T cells (FIG. 3D), but its weight showed only a minor change with inflammation (adjusted P=0.002, Mann-Whitney U test, FIG. 3D). This suggests that activation of type 2 inflammation in this model did not significantly interfere with the activity of regulatory T cells.

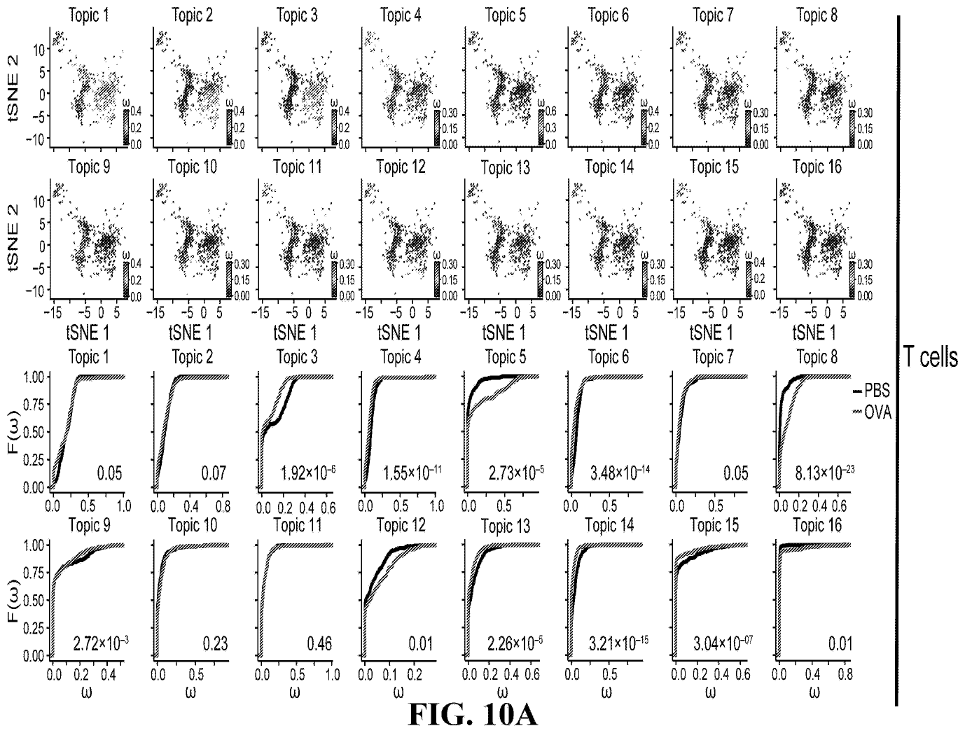

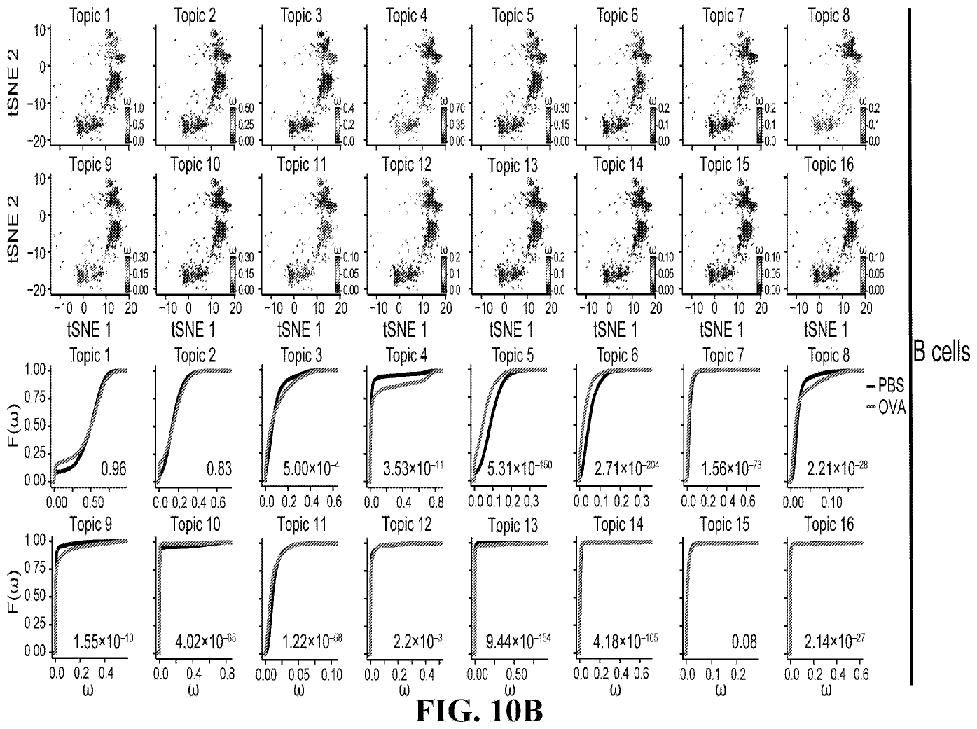

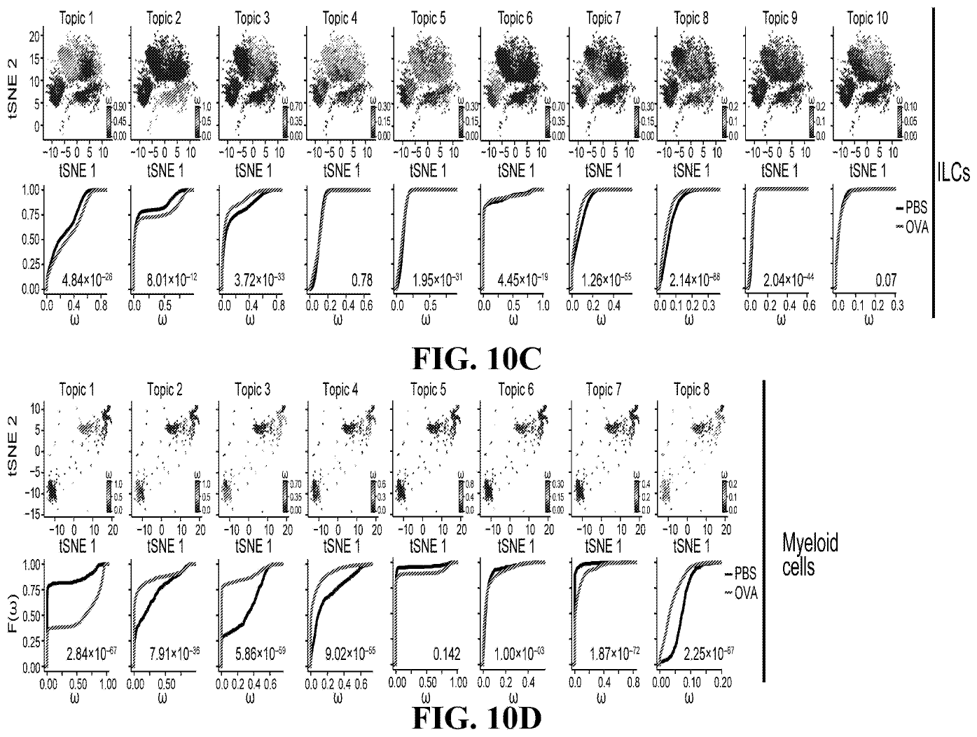

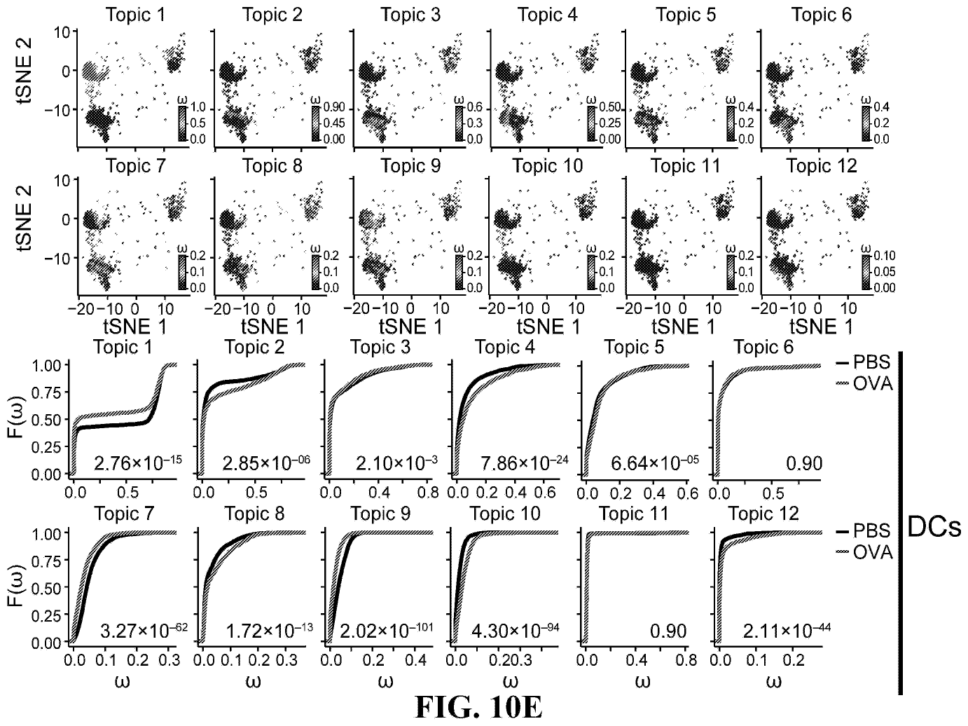

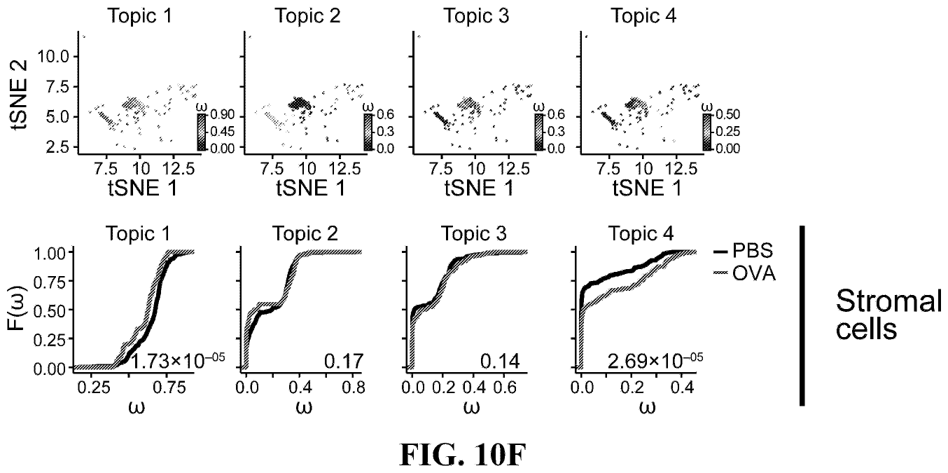

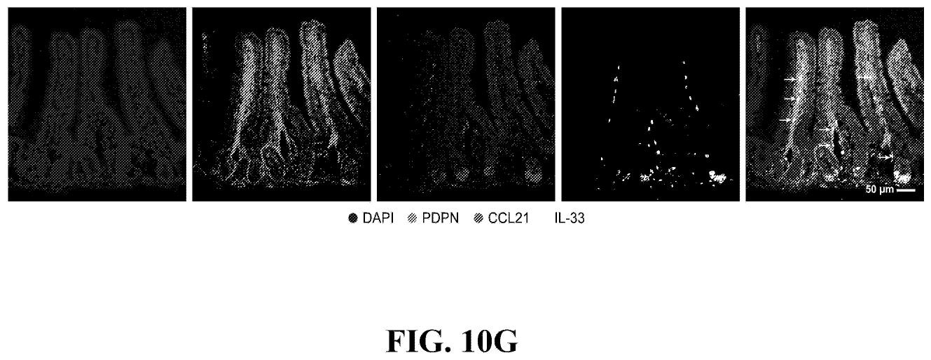

Topic 4 from stromal cells, defined by the expression of the type 2 inflammatory cytokine 1133 and key chemokines such as Ccl21a, was uniquely associated with fibroblasts and increased in weight with inflammation (FIG. 3E, adjusted P=2.69*10⁻⁵, Mann-Whitney U test). Applicants confirmed that IL-33 and CCL21 were co-expressed in PDPN⁺ stromal cells by immunofluorescence (FIG. 10G). Moreover, the number of IL-33⁺PDPN⁺ intestinal fibroblasts was significantly increased in mice under type 2 inflammation induced by OVA (FIG. 3F, P=0.002, t-test), indicating that stromal cell-derived IL-33 may play critical roles in promoting intestinal type 2 inflammation.

### Example 4—Calca-Encoded α-CGRP Expression is a Key Feature of the ILC2 Activation Program Induced by Inflammation, but its Receptor is Repressed by Inflammation

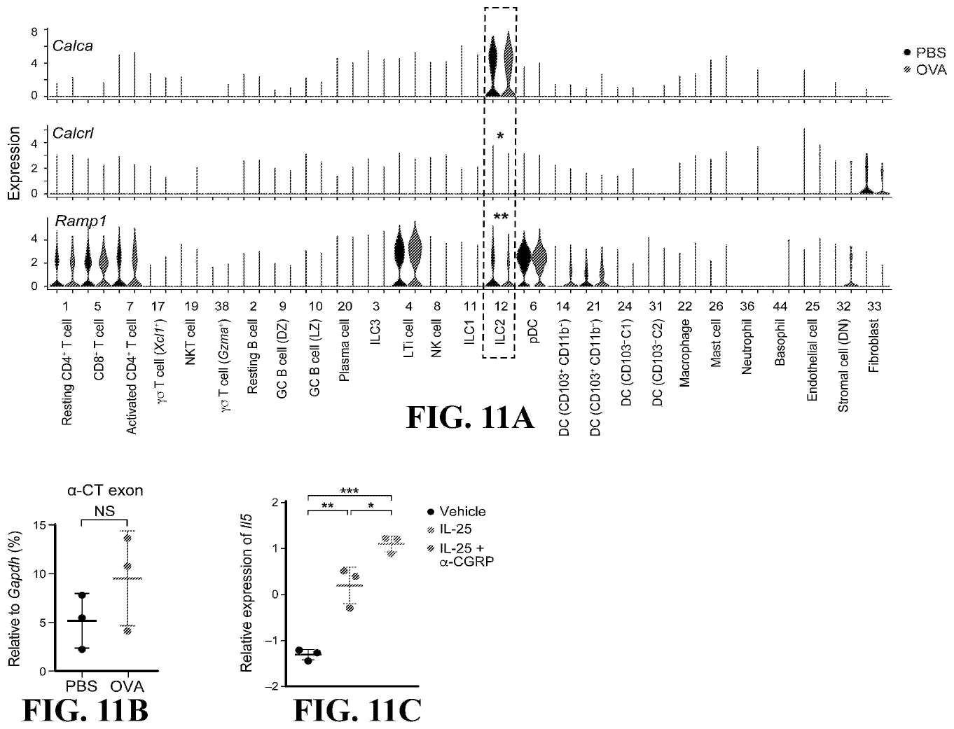

Calca was the highest and most uniquely scoring gene in the ILC2 activation program (FIG. 3B) and Calca transcript levels were highest in ILC2s among all cell types in the dataset (FIGS. 4A and 11A). Exons 4 and 5 of Calca encode the neuropeptides calcitonin (CT) and α-CGRP, respectively, via alternative splicing (Amara et al., 1982). Quantitative PCR (qPCR) revealed that while CT-encoding exon 4 was expressed in intestinal KLRG1⁺ILC2s at steady state (FIG. 11B), α-CGRP-encoding exon 5 was detectable only following induction of a type 2 inflammatory reaction to OVA (P=0.01, t-test) (FIG. 4B). Conversely, the corresponding transcripts of α-CGRP receptor components—a G-protein-coupled receptor complex consisting of CRLR and RAMP1—were expressed in ILC2s at steady state (FIGS. 4B and 11A), but significantly repressed by type 2 inflammation (FIG. 4B, P=0.005 and 0.02 for Calcrl and Ramp 1, respectively, t-test). The Calcr transcript, encoding the CT receptor, was not detectable by either scRNA-seq or qPCR (data not shown). Taken together, these results are consistent with a negative feedback model, where signaling through α-CGRP to ILC2s suppresses its receptor's expression in the cells, and suggest that an α-CGRP-mediated signaling pathway may regulate ILC2 activity in type 2 immune responses.

### Example 5—α-CGRP Suppresses Type 2 Inflammation-Induced Activation and Expansion of ILC2s In Vitro and In Vivo

To determine if α-CGRP can directly trigger signaling in ILC2s and impact their transcriptional states under inflammatory conditions, Applicants performed bulk RNA-seq of KLRG1⁺ ILC2s isolated from the small intestine and stimulated in vitro with IL-25 alone or together with α-CGRP. As expected, IL-25 alone induced the expected activation phenotype, including expression of type 2 cytokines, such as Il13 and Il5, and key mitotic genes, such as Myc (FIG. 4C). Strikingly, this IL-25-induced activation signature was significantly suppressed when cells were treated with both IL-25 and α-CGRP (FIG. 4C,D, adjusted P<0.001, t-test), with the exception of a few genes, notably Il5, which was further induced (FIGS. 4C and 11C). Moreover, α-CGRP co-treatment significantly suppressed IL-25-induced cell division (FIG. 4E, P=0.01, t-test). Thus, α-CGRP inhibits IL-25-induced proliferation and activation of intestinal KLRG1+ILC2s.

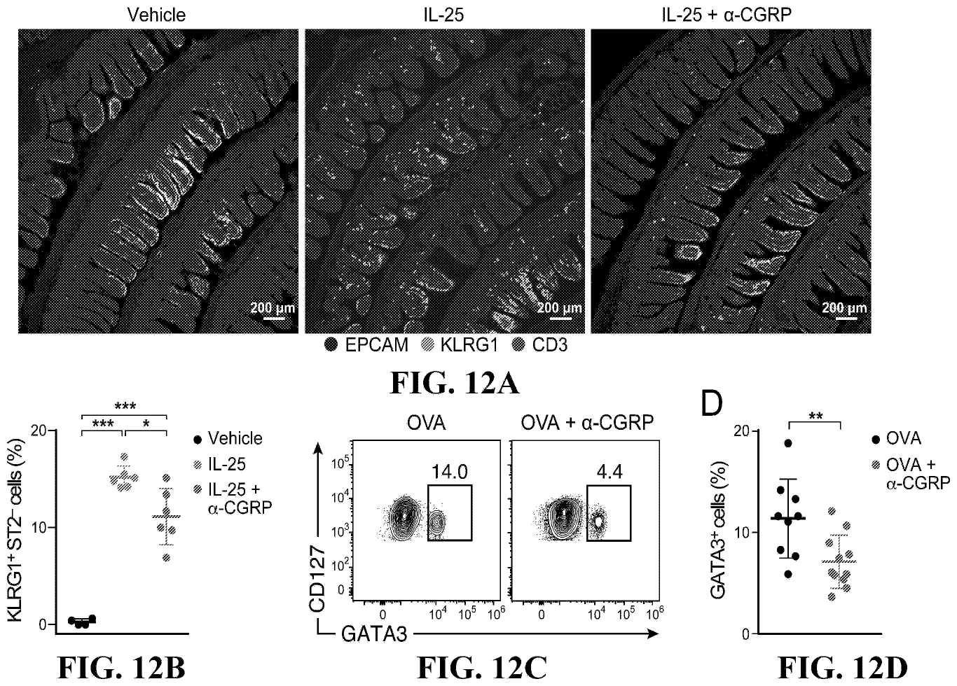

Applicants next used an IL-25-induced in vivo activation model (Huang et al., 2015) (FIG. 5A) to show that α-CGRP suppresses expansion of ILC2s responding to inflammation. Intraperitoneal administration of α-CGRP impaired IL-25-induced expansion of KLRG1⁺ ILC2s (FIGS. 5B and 12A), consistent with the in vitro results. Recent work has shown that intestinal KLRG1⁺ ILC2s can migrate to mesenteric lymph nodes (mLNs) and other tissues during inflammation via sphingosine 1-phosphate (S1P)-mediated chemotaxis (Huang et al., 2018). To determine whether α-CGRP primarily impacts intestinal or mLN ILC2s, Applicants relied on the fact that intestinal GATA3⁺ ILC2s are generally phenotypically KLRG1⁺ST2⁻ (data not shown), whereas mLN ILC2s are mainly ST2⁺ at steady state (FIG. 5C). IL-25 administration increased the frequency of both KLRG1⁺ST2⁻ inflammatory ILC2s and ST2⁺ ILC2s in mLNs (FIGS. 5C-E and 12B). But co-administration with α-CGRP only reduced the frequency of KLRG1⁺ST2⁻ILC2s (FIGS. 5C,D and 12B), which are cells that migrated from the small intestine to the mLNs, but not of ST2⁺ ILC2s (FIG. 5E). Thus, α-CGRP preferentially regulates the response of intestinal KLRG1⁺ST2⁻ ILC2s. Furthermore, α-CGRP also significantly suppressed the expansion of intestinal ILC2s in a food allergy model (FIGS. 5F,G and 12C,D, P=0.02 for 5G, P=0.009 for S5D, t-test), where Applicants intragastrically administrated α-CGRP together with OVA into pre-sensitized mice. Taken together, α-CGRP antagonized expansion of intestinal KLRG1⁺ ILC2s in two different type 2 inflammatory models.

### Example 6—at Homeostasis, α-CGRP is Predominantly Expressed In Vivo in Two Subsets of Chat⁺ Enteric Neurons

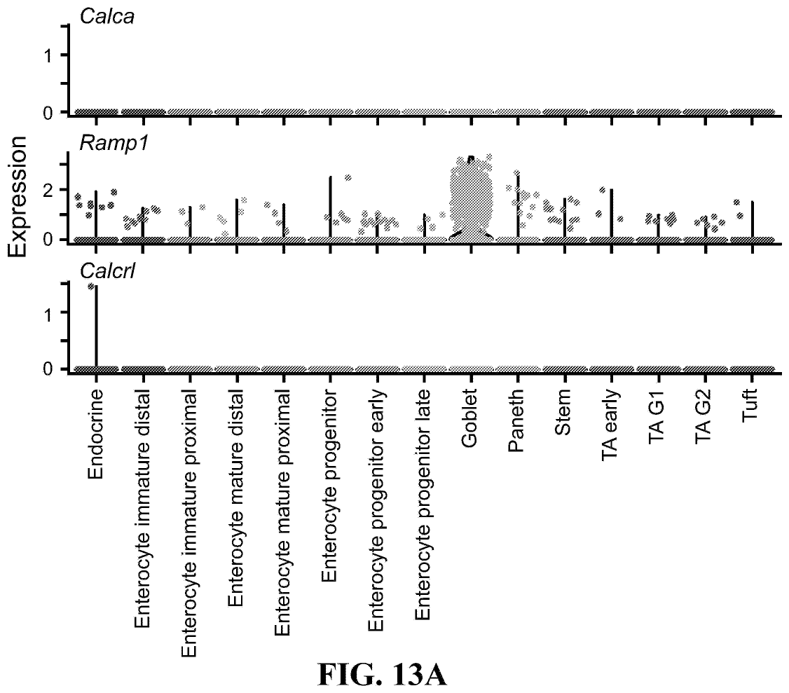

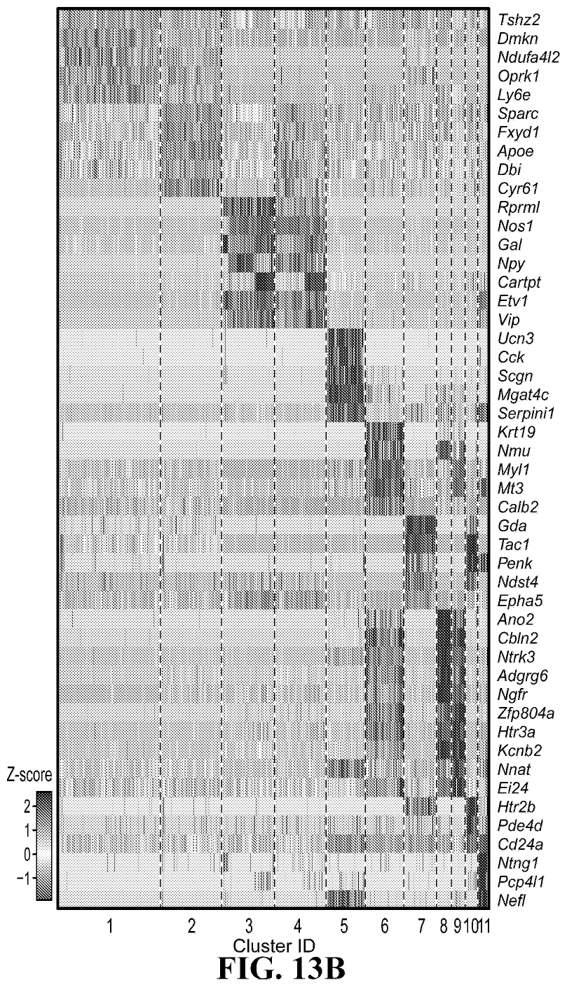

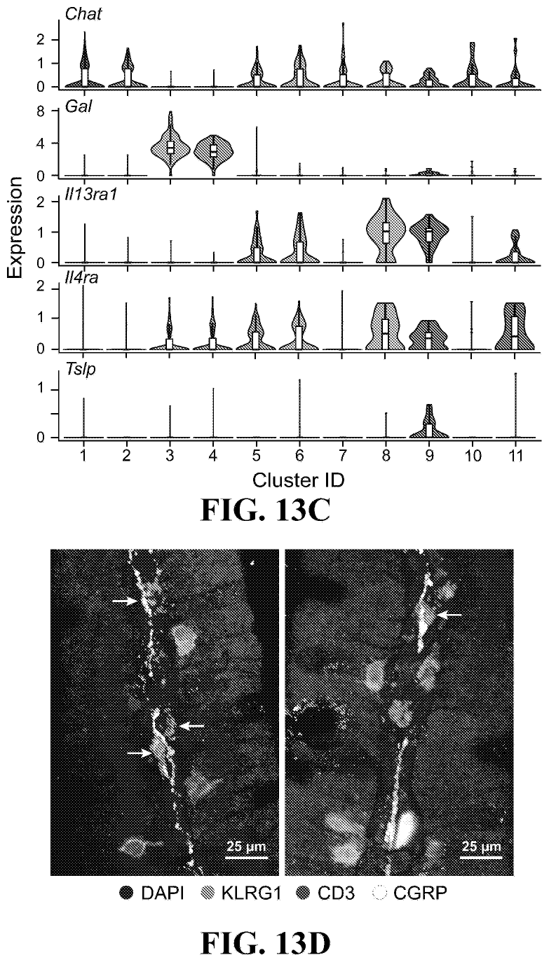

Without inflammation, intestinal KLRG1⁺ ILC2s expressed CGRP receptors (FIG. 4B), but not CGRP, leading us to search for neighboring cell type(s) which potentially produce CGRP at homeostasis. To this end, Applicants analyzed Calca gene expression across multiple intestinal scRNA-seq datasets, including the previous epithelial atlas for the mouse small intestine epithelium (Haber et al., 2017) (FIG. 13A), the intestinal immune atlas here (FIG. 1), and scRNA-Seq of enteric neurons from Wnt1-Cre:R26Tomato mice (Zeisel et al., 2018) (FIGS. 6A and 13B,C). Calca expression was only detected in two subsets of ChAT⁺ enteric neurons (FIGS. 6B and 13C). These ChAT⁺ enteric neurons also expressed (FIG. 6B): (1) the CGRP receptor genes, (2) the Calcb gene, encoding β-CGRP peptide, which differs in two amino acid from α-CGRP, but has not been substantially studied for its function in immune cells, and (3) the Nmu gene, which Applicants and others have shown amplifies the activity of ILC2s under type 2 inflammation in the lung and small intestine (Cardoso et al., 2017; Klose et al., 2017; Wallrapp et al., 2017).

Applicants confirmed by immunofluorescence staining that ChAT⁺ enteric neurons expressed CGRP (FIG. 6C), and that ILC2s contact with CGRP-expressing neurons at the steady state (FIG. 13D), consistent with previous reports that ILC2s are closely associated with neurons in the intestine (Klose et al., 2017). Thus, CGRP from these ChAT⁺ enteric neurons may regulate ILC2 homeostasis.

### Example 7—α-CGRP Suppresses ILC2 Proliferation Through Activating a cAMP Response Module

The presence at homeostasis of α-CGRP receptors on ILC2s and α-CGRP's expression on ChAT⁺ intestinal neurons led Applicants to hypothesize that it has an effect on ILC2s during homeostasis. To test this hypothesis, Applicants next measured the expression profiles of intestinal KLRG1⁺ ILC2s stimulated in vitro by α-CGRP alone, and identified genes differentially expressed under this signal (FIG. 6D, Methods).

Analyzing the profiles suggested that α-CGRP activated a cAMP response module. First, α-CGRP stimulation induced expression of key genes involved in adenylate cyclase-mediated GPCR signaling, including Adrb2, Adora2a, Pde4b and Akap12 (FIG. 6D), as well as cAMP response genes, including Areg (Shao and Sheng, 2010), Crem, and Il5 (FIG. 6D). Indeed, previous studies showed that cAMP directly induces IL5 expression in ILC2s (Nussbaum et al., 2013). Consistently, ATAC-Seq profiling of ILC2s revealed that regions of increased chromatin accessibility following α-CGRP stimulation alone where enriched for gene loci involved in the adenylate cyclase pathway (FIG. 6E and Table 5).

Moreover, α-CGRP stimulation in vitro negatively regulated ILC2 proliferation, and this effect may be mediated by induction of genes involved in cell cycle arrest in the cAMP response module. In the KLRG1⁺ ILC2 cells, α-CGRP induced expression of genes involved in cell cycle arrest, such as Cdkn1a, Gadd45a and Akap12, as well as of key negative regulators of ILC2 expansion, Adrb2 and Pdcd1 (Moriyama et al., 2018; Taylor et al., 2017) (FIG. 6D). cAMP and its principal target, cAMP-dependent protein kinase (PKA), have been previously shown to suppress proliferation via negatively regulating mitogen-activated protein (MAP) kinase cascade in a cell context-dependent manner (Stork and Schmitt, 2002). Indeed, the adenylate cyclase activator forskolin, which increases intracellular cAMP levels (Rodriguez et al., 2013), suppressed the proliferation of ILC2s (FIG. 6F), but not their viability (FIG. 6G). Furthermore, treating ILC2s with an adenylate cyclase inhibitor (Klein et al., 2012) partially rescued the cell proliferation defect mediated by α-CGRP (FIG. 6H). Taken together, the data suggest that α-CGRP suppresses ILC2 proliferation via activating a cAMP response module.

### Example 8—At Homeostasis, α-CGRP Maintains ILC2 and Type 2 Responses In Vivo

Finally, Applicants tested if α-CGRP-mediated signaling regulates homeostasis of intestinal KLRG1⁺ ILC2s at steady state in vivo, by analyzing α-CGRP exon knockout (α-CGRP KO) mice, in which the CT exon of Calca gene remains intact (Oh-hashi et al., 2001). The frequency of intestinal KLRG1⁺ILC2s was significantly increased in α-CGRP KO mice compared to wild type (WT) controls (P=0.002, t-test) (FIG. 6I). Type-2 cytokines promote differentiation of tuft cells (Biton et al., 2018; Gerbe et al., 2016; Howitt et al., 2016; von Moltke et al., 2016), and the number of tuft cells was indeed also significantly increased in α-CGRP KO mice (P=0.036, t-test) (FIG. 6J). Thus, α-CGRP-mediated signaling helped maintain the homeostasis of KLRG1⁺ ILC2s and type 2 immunity in the small intestine.

### Example 9—Discussion

Here, Applicants collected and analyzed a scRNA-seq atlas of immune cells in the small intestine in homeostasis and during type 2 inflammation, uncovered dynamic responses in cell type specific programs that monitor and titrate mucosal responses, and identified α-CGRP as a key regulator impacting the frequency and activity of intestinal KLRG1⁺ ILC2s.

This work highlights a role for α-CGRP in regulating ILC2s at both homeostasis and during inflammation. Previous studies showed that CGRP signaling can generate both pro- and antiinflammatory immune responses, depending on cell type, tissue and experimental model (Assas et al., 2014). α-CGRP increases IL-5 expression in lung ILC2s co-stimulated with IL-33 (Sui et al., 2018). Mice with deficiency in CRLR in IL-5-expressing cells manifest a normal frequency of ST2⁺ILC2s in lungs exposed to house dust mite (HDM) (Sui et al., 2018), consistent with the finding that ST2⁺ ILC2s in mLNs are not significantly affected by α-CGRP administration. However, this work further reveals that α-CGRP reduced the expansion of intestinal ST2⁻KLRG1⁺ ILC2s in two different inflammatory models. Although differences in cell states between lung ST2⁺ and intestinal ST2⁻KLRG1⁺ ILC2s (Ricardo-Gonzalez et al., 2018) might contribute to their differential reactions to CGRP signaling, additional studies in animal models where CRLR is specifically-deleted in intestinal KLRG1⁺ ILC2s will be needed to elucidate the full molecular mechanism. In addition, it will be important to compare the effects of α-CGRP vs. β-CGRP binding to CRLR in ILC2s.

Applicants show that two subsets of ChAT⁺ enteric neurons are the predominant source of CGRP in the small intestine at steady state. In addition to multiple neuropeptides, these ChAT⁺ neurons also express Il13ra1 and Il4ra (FIG. 13C), which encode the components of the receptor complex for the type 2 cytokine IL-13. Thus, these ChAT⁺ neurons may be specialized for crosstalk with the immune system. Future studies will help determine if cytokine signaling from immune cells during inflammation further boosts the secretion of neuropeptides.

CGRP signaling is an additional key axis of neuro-immune interaction with implications for inflammation, including food allergy. Neuronal signals are emerging as important orchestrators of immune responses in the gastrointestinal tract (Chesne et al., 2018; Godinho-Silva et al., 2018; Veiga-Fernandes and Mucida, 2016). In particular, the nervous system has been shown to exercise dual functions to either activate or inhibit ILC2s via different GPCRs. While NMUR1 signaling amplifies ILC2 activation in the lung and intestines (Cardoso et al., 2017; Klose et al., 2017; Wallrapp et al., 2017), the β2-adrenergic receptor (β²AR) pathway negatively regulates ILC2 expansion (Moriyama et al., 2018).

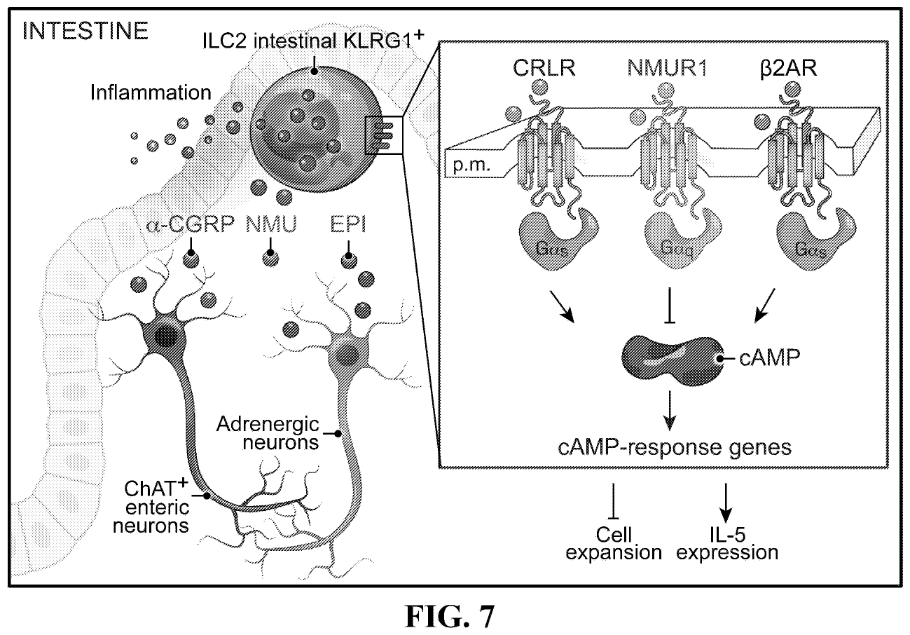

Applicants thus propose a model for how ILC2s integrate and balance such diverse neural signaling cues, involving different Ga proteins and their downstream signaling (FIG. 7). In this model, the amplifier NMUR1 mainly signals through Gα_{q/11} and inhibits cAMP levels (Martinez and O'Driscoll, 2015), whereas the negative regulators CRLR and β²AR are both coupled with Gαₛ proteins that activate adenylate cyclase and the cAMP pathway (Chatterjee et al., 1993; Madamanchi, 2007). Intracellular cAMP accumulation induces expression of the cAMP response module including IL-5, but suppresses ILC2 proliferation. Thus, selective activation of different subgroups of a subunits of G proteins coupled with variable downstream effects, including the concentration of cAMP, might be utilized by ILC2s to fine-tune the response to neuronal signaling.

Collectively, this work underscores the importance of α-CGRP suppression of ILC2 activity, which may serve as a therapeutic target for treating diseases like food allergy. Since monoclonal antibodies against CGRP have been recently approved to treat migraine (Edvinsson, 2018), it will be important to monitor for the incidence of allergic diseases in treated patients. More broadly, this work provides a resource for understanding the intestinal immune system in response to type 2 inflammation in the context of each specific cell type. Further exploration of the atlas can lead to additional hypotheses on circuits within and between additional cell types that contribute to type 2 immune responses.

### Example 10—Experimental Methods

Mice. BALB/cJ mice (Jax 000651) were obtained from the Jackson Laboratory. α-CGRP knock out mice (B6.12956-Calca<tm1Hku>) were kindly provided by Dr. Vijay K. Kuchroo (Brigham and Women's Hospital, Boston, Mass., USA). Mice were housed in specific pathogenfree conditions and were used and maintained in accordance with the Institutional Animal Care and Use Committee (IACUC) protocol #0055-05-15.

To induce allergic reaction to OVA, 6 to 7 weeks old mice were sensitized twice, two weeks apart, with 50 μg of OVA plus 1 mg of aluminum potassium sulfate adjuvant via intraperitoneal injection. Two weeks later, mice were orally administered with 50 mg of OVA on every other day for a total of five times. 1 μg of α-CGRP peptide was administered simultaneously when indicated. Mice were deprived of food for 3-4 hours in cages with wood chip bedding for limiting antigen degradation in the stomach before each intragastric challenge.

To activate ILC2s in vivo, 200 ng of IL-25 was intraperitoneally injected into 7-10 weeks old mice daily for two days. 1 μg α-CGRP was injected together as noted.

Isolation of cells from Peyer's patches (PPs). Peyer's patches (PPs) were carefully dissected from the small intestine under a stereo microscope using a fine scissor, pierced once with a fine forcep. Tissues were digested in freshly made digestion buffer (RPMI-1640 containing 100 μg/ml Liberase™ and 50 μg/ml DNase I at 37° C. on a roto-mixer. After 15 min, tissues were very gently mixed using a 1 ml pipette. The supernatant was collected and added to ice-cold MACS buffer (pH 7.4; PBS plus 2% FCS and 2 mM EDTA). Pre-warmed (37° C.) fresh digestion buffer was added to the remaining tissues. After rotation at 37° C. for 15 min, the mixture was vigorously mixed using a 1 ml pipette for 1 min. Supernatants from the two steps were combined and passed through 70 μm filters and stained for FACS (below).

Isolation of cells from the lamina propria (LP). The small intestines were opened longitudinally and washed in ice-cold PBS. For scRNA-seq, roughly 0.5 cm of fragments from each of the distal, middle and proximal regions without visible PPs were collected. Epithelial cells were dissociated by tissue rotation in pre-warmed (37° C.) PBS containing 10 mM EDTA at 37° C. for 15 min, followed by additional incubation on ice for 15 min. Tissues were then shaken vigorously. After washing twice with PBS containing 2% FCS, tissues were digested in pre-warned (37° C.) digestion buffer at 37° C. on a roto-mixer for 25 min. The supernatants were then passed through 70 μm filters and stained for FACS.

For experiments other than scRNA-seq, the entire small intestine was cut into 1 cm pieces after eliminating PPs. Epithelial cells dissociation was performed by stirring tissues in flasks containing PBS and 10 mM EDTA on a magnetic stirrer at 37° C. two times for 15 min. After vortex, the tissue was stirred in pre-warmed (37° C.) digestion buffer for 25 min at 37° C. The supernatant was passed through 100 μm filter. Leukocytes were further enriched by a 40%/70% Percoll gradient centrifugation before flow staining.

Flow cytometry and cell sorting. Cells were washed and suspended in MACS buffer (pH 7.4; PBS plus 2% FCS and 2 mM EDTA). Nonspecific antibody binding was blocked with CD16/CD32 (2.4G2) antibody for 15 min on ice. Cells were then stained with antibody cocktails for 30 min at 4° C. Lineage-positive cells were excluded for analyzing ILCs by staining for CD3ε (145-2C11), CD5 (53-7.3), CD19 (6D5), CD11b (M1/70), CD8 (53-6.7), CD11c (N418), Gr-1 (RB6-8C5), TCRγδ (eBioGL3 (GL-3, GL3)) and TCRβ (H57-597). To analyze mast cells, lineage-positive cells were excluded by staining for CD3ε, CD5, CD19, CD11c and SiglecF (E50-2440). For surface staining, antibodies for KLRG1 (2F1), CD45 (30-F11), CD127 (A7R34), CCR6 (29-2L17), NKp46 (29A1.4), CD90.2 (53-2.1), FcεRI (Mar-1), IgD (11-26c.2a) and ST2 (DJ8) were used. For intracellular staining, cells were fixed and permeabilized using the Foxp3 transcription factor staining buffer set, followed by staining with anti-GATA-3 (TWAJ) or anti-RORγt (B2D) antibodies. Dead cells were excluded with 7-AAD or Fixable Viability Dye eFluor 780. Flow cytometry was performed on Cytoflex (Beckman Coulter) and analyzed with FlowJo software. Sorting was performed with the SH800S Cell Sorter (Sony Biotechnology).

Droplet-based scRNA-seq. Single cells were captured via the GemCode Single Cell Platform using the GemCode Gel Bead, Chip and Library Kits (10× Genomics), according to the manufacturer's protocol. Briefly, flow-sorted cells were suspended in PBS containing 0.4% BSA, and loaded at 7,000 cells per channel. The cells were then partitioned into GemCode instrument, where individual cells were lysed and mixed with beads carrying unique barcodes in individual oil droplets. The products were subjected to reverse transcription, emulsion breaking, cDNA amplification, shearing, 5′ adaptor and sample index attachment. Libraries were sequenced on a HiSeq 2500 (Illumina).

Quantitative real-time PCR. RNA was isolated from 10,000 cells per sample using PicoPure RNA Isolation Kit, according to the manufacturer's protocol and reverse transcribed to cDNA with iScript cDNA Synthesis Kit. Gene expression was analyzed by quantitative real-time PCR on a ViiA7 System (Thermo Fisher Scientific) using iTaq™ Universal SYBR® Green Supermix with the indicated primers. Expression values were calculated relative to Gapdh detected in the same sample by qPCR.

ILC2 culture. Sort-purified intestinal KLRG1⁺ ILC2s were incubated in RPMI supplemented with 10% FCS, 10 mM Hepes, 1 mM sodium pyruvate, 10% FBS, 80 μm 2-mercaptoethanol, 2 mM glutamine, 100 U/ml penicillin, 100 m/ml streptomycin, 100 ng/ml IL-2 and 100 ng/ml IL-7 in 96-well round bottom plate at 37° C. and 5% CO2. If indicated, the culture was supplemented with 100 ng/ml IL-25. For the in vitro proliferation assay, cells from different mice were pooled and labeled with CellTrace Violet, and then cultured at 1,000-1,500 cells per well with 10 μg/ml α-CGRP for 60 hours. When indicated, 10 μM forskolin or 1 μM SQ 22, 536 in DMSO was supplied and cells were cultured for 40 hours. For bulk RNA-seq, cells from individual mice were stimulated at 200 cells per well with 0.4 μg/ml α-CGRP for 3 hours. For bulk ATAC-seq, cells were stimulated with 0.4 μg/ml α-CGRP for 2 hours.

Immunofluorescence staining and imaging. The small intestines were fixed in 2% PFA, embedded in O.C.T. Compound and sliced into 10 μm by frozen section. The slides were blocked with 0.1% Triton X-100, 2% FCS and donkey serum. The staining reagents incudes Alexa Fluor 594 anti-IgD (11-26.2a), Alexa Fluor 647 anti-CD138 (281-2), eFluor 450 anti EPCAM (G8.8), Alexa Fluor 594 anti-CD3ε (17A2), Alexa Fluor 488 anti-Podoplanin (eBio8.1.1 (8.1.1)), rat anti-CCL21/6 (59106), goat anti-IL-33 (polyclonal), hamster anti-KLRG1 (2F1), rat anti-CGRP (polyclonal), goat anti-ChAT (polyclonal), rabbit anti-DCAMKL1 (polyclonal), Alexa Fluor 647-donkey anti-goat IgG, Alexa Fluor 647 donkey anti-goat IgG, Cy™3 donkey anti-rat IgG, Alexa Fluor 488 goat antisyrian hamster IgG (H+L), Alexa Fluor 647 goat anti-rabbit IgG (H+L) and DAPI. Slides were mounted with the ProlongGold Antifade reagent and examined with a ZEISS LSM 710 upright microscope using ×10, ×20 air or ×60 oil immersion lens. The images were analyzed with ImageJ.

Bulk RNA-seq. 200 ILC2s from each condition were lysed in 10 ul TCL buffer plus 0.5% 2-Mercaptoethanol. Libraries were processed with SMART-Seq2 (Picelli et al., 2013) with at least three replicates per condition, and paired-end sequenced (75 bp×2) with a 75 cycle Nextseq 500 high output V2 kit.

Reads were aligned to the mouse reference genome (NCBI 38, mm10) using Bowtie (Langmead et al., 2009) with default parameters, and expression abundances were estimated using RSEM software (Li and Dewey, 2011). The differential gene expression analysis was performed with edgeR (Robinson et al., 2010) with default parameters.

Bulk ATAC-seq. ATAC-seq experiment was performed using a published protocol (Buenrostro et al., 2013) with minor modifications. Briefly, 2,000 cells in 5 μl PBS, 17.3 μl H²O and 25 μl of transposition buffer (66 mM Tris-acetate, 132 mM K-acetate, 20 mM Mg-acetate, 32% DMF, and 0.2% NP-40) were mixed and incubated at room temperature for 10 min. After adding 2.5 μl of Tn5 transposase to the reaction, the transposition was carried out at 37° C. for 30 min with gentle shaking at 300 rpm and then purified with Zymo DNA Clean and Concentrator (Zymo Research). The library was amplified for 11 cycles, purified with Zymo DNA clean (Zymo Research), and sequenced on an Illumina Next-seq platform using 75 cycle Nextseq 500 high output V2 kit (Read 1: 38 cycles, Index 1: 8 cycles, Index 2: 8 cycles, Read 2: 38 cycles).

The reads were trimmed and aligned to the mouse reference genome (mm9) with Bowtie2 aligner (Langmead and Salzberg, 2012) using the option-X2000. Then, Applicants discarded reads with alignment quality<Q30, improperly paired, mapped to the unmapped contigs, chrY, and mitochondria. Duplicates were removed using Picard tools (function MarkDuplicates, broadinstitute.github.io/picard/). MACSv2 peak caller (Zhang et al., 2008) (version: 2.1.1) was used to call accessible regions of open chromatin regions (ATAC-Seq peaks) with the following parameters (-nomodel -nolambda -keep-dup -call-summits). Peaks overlapping with ENCODE blacklisted regions were filtered out using BEDtools (function itersectBed). Peak summits were extended by ±250 bp, and fragment counts in peaks were calculated using chromVAR (Schep et al., 2017) (version: 1.1.1). Peaks were allocated to genes using GREAT (McLean et al., 2010) (version: 3.0.0) with “basal plus extension” association rule with default parameters. Functional enrichment analysis was performed using GREAT (version: 3.0.0).

### Example 11—Computational Methods

scRNA-seq data QC and pre-processing. Reads were aligned to the mouse reference genome (NCBI 38, mm10) using Cell Ranger v2.1.1 (10× Genomics) to generate cell-gene count matrices. After removing cells with less than 500 UMIs and high mitochondrial RNA UMIs (more than four times of the median number of mitochondrial UMIs across cells), Applicants obtained 36,797 cells from PP regions (15,939 cells from OVA-allergic mice and 20,858 cells from controls), and 21,270 cells from LP (11,405 cells from OVA-allergic mice and 9,865 cells from controls). 19,221 genes were retained after filtering genes expressed in less than five cells.

Applicants expected batch effects in the data, because libraries were prepared and sequenced at different times, and because Applicants used two sorting strategies to remove either IgD⁺ Naive B cells or CD19+/CD3⁺ cells. Moreover, the number of recovered cells varied across experiments; for example, Applicants recovered 10,567, 6,439, 5,016, 4,571, and 10,204 cells from each of the five experiments for cells from PPs. Applicants explored several possibilities to address these confounders. Two recent methods have been developed to align scRNA-seq data from different batches (Butler et al., 2018; Haghverdi et al., 2018). However, a cell type must be shared by all datasets for Seurat's CCA approach to correctly align cells (Butler et al., 2018), which is not appropriate for this case. mnnCorrect (Haghverdi et al., 2018) can merge datasets with private (distinct) cell types, but in the datasets where there were dozens of cell types, mnnCorrect successfully merged only some of these clusters, and failed to align cells from other clusters. (More recent methods (Korsunsky et al., 2018; Lin et al., 2018; Stuart et al., 2018; Welch et al., 2018) might help merge cells from different experiments but were published when this analysis was long completed.)

Principal Component Analysis (PCA) is typically used to extract a small number of features (principal components (PCs)) from a normalized gene-cell count matrix. These feature vectors were used for clustering analysis or as inputs for visualization. Unfortunately, in the presence of batch effects, some of the top features typically captured batch effects (Chen et al., 2011). Therefore, batch effects could not be removed by discarding the features with small eigenvalues in PCA.

To help address these batch effects, Applicants took an alternative approach, where Applicants projected the scRNA-seq data to a reference dataset consisting of microarray measurements of immune cells from 276 samples (Heng et al., 2008). Specifically, Applicants first did PCA on the microarray data and extracted the first 101 eigenvectors. Applicants discarded the first eigenvector as the corresponding first PC was correlated with batch information in the microarray data. Applicants next projected the scRNA-seq data to the 100-dimensional space spanned by the eigenvectors (PCs 2-101) from the microarray data. The coordinates of cells in the 100-dimensional space were used for clustering and as inputs of scvis.

scRNA-seq clustering of PP cells. Applicants used the Louvain community detection algorithm (Blondel et al., 2008; Levine et al., 2015) to cluster cells from Peyer's patch regions. As the Louvain clustering algorithm tends to miss some small clusters, Applicants used densityCut (Ding et al., 2016) to find the likely cluster centers. For each center, Applicants changed the edge weights (w′) connecting the cluster center to its neighbors to (w′)=2 kw, where k is the number of nearest neighbors in the k-nearest neighbor (k-NN) graph, and w is the original edge weight of an edge. Louvain clustering on this edge re-weighed graph produced 46 clusters.

Next, Applicants assessed the robustness of the cell to cluster assignment. For each cluster and each batch, Applicants computed the ‘bulk’ gene expression profile of the cells from a given batch in the cluster. The ‘bulk’ gene expression profile for a set of cells is computed by first taking the sum of the gene expression vectors from these cells, where the gene expression vector of a cell was the raw UMI count vector, one element for a gene. The dimensionality of a gene expression vector was the number of genes. To make the bulk gene expression vectors from different sets of cells comparable, a bulk gene expression vector was normalized by dividing the total number of UMIs from all the cells used in computing that bulk gene expression vector, and further multiplying by 10⁴ and finally taking the log transform (adding one before the log transformation to make all the elements of the bulk vector positive). Then, for each cell, Applicants computed its Pearson correlation coefficient with the “bulk” profile for each cluster. Applicants denote the maximum correlation between a cell x and the bulk profiles of cluster i (from different batches) as c′. If cell x is originally assigned to cluster j, and c^{j}<0.9c^{k}, then Applicants reassign cell x to cluster k. Only ˜0.26% (97 of 36,797) cells were re-assigned to clusters different from their original assignment. Applicants also tested whether two clusters should be merged, if there are no more than 10 significantly differentially expressed genes between them (with differential expression estimated with the Wilcoxon rank-sum test in the package Seurat (Butler et al., 2018)). However, none of the 46 clusters required merging by this criterion.

Clustering cross-validation of PP cells. Applicants performed a 10-fold cross validation analysis on the PP data to evaluate the quality of the clustering. Applicants partitioned the PP data into ten approximately equal size sets, and trained a knearest neighbor (k-NN) classifier (Applicants used a small k=11 as some clusters are small, e.g., 17 cells in cluster 46) on nine folds of data, leaving one fold of data for testing. Applicants repeated this training and testing scheme ten times such that all the data points were used for testing only once.

The k-NN classifiers had a high overall accuracy of 97.2%. Classification accuracy varied for cells from different clusters, with those from cluster 39 having the lowest accuracy of 65.6%. Cluster 39 consisted of a mixture of low-quality plasma B cells and cell doublets (macrophage and epithelial cell doublets). A subgroup of cells expressed plasma B cell marker genes, such as Jchain and Mzb1, but had a relatively small number of UMIs per cell compared to the cells from the plasma cell Cluster 20 (FIG. 8D). Some of these low-quality plasma B cells also had high mitochondrial UMI ratios. Another subset of cells in Cluster 39 expressed both epithelial cell markers (e.g., Epcam and Krt8) and macrophage marker genes (e.g., Lyz1 and Lyz2). These cells had a very large number of UMIs per cell (FIG. 8D) and likely represented potential macrophage and epithelial cell doublets. Notably, although the number of cells in each cluster was extremely unbalanced (e.g., 6,948 cells in cluster 1 but only 17 cells in cluster 46), the classification accuracies were largely uncorrelated with the number of cells in each cluster.

Cluster annotation and filtering of PP cells. Applicants next used MAST (Finak et al., 2015) to identify significantly up-regulated marker genes for each of the 46 clusters, accounting for batch (experimental dates) and the scaled number of detected genes in each cell as covariants (Soneson and Robinson, 2018). Based on known function of the marker genes, Applicants annotated 6 major cell lineages/groups: T cells, B cells, dendritic cells, ILCs, myeloid cells and stromal cells.

Applicants conservatively excluded from further analyses the smallest clusters (<0.05% of all cells) and several ambiguous clusters that Applicants could not confidently assign with cell identities. Applicants note that the small clusters may be biologically valid, but the small number of cells limits the ability to further study them here. For example, the top markers of cluster 45 (18 cells) included Dntt, Rag1, Chrna9, Tctex1d1, Arpp21, which are highly expressed in progenitors of T cells at the double-positive stage in Immgen (Heng et al., 2008; Painter et al., 2011). Cluster 46 (17 cells) may consist of lymph node lymphatic endothelial cells, as they expressed their known marker genes such as Lyve1, Prox1, and Cp. Experimental validations are required for confidently including them in the downstream analyses. Cells in Cluster 34 (100 cells) from PP, expressed both pDC and myeloid gene markers. Only two marker genes (Gtf2a1 and 2310001H17Rik) overlapped between Cluster 34, and either of its adjacent clusters in scvis, clusters 6 and 22 (FIG. 1B). Gtf2a1 was expressed lowly in about 25% of the cluster 34 cells and 2310001H17Rik was expressed in several other cell types, such as T cells and neutrophils. Applicants annotated cluster 34 as ‘Unresolved’. Cluster 18 and cluster 37 were also annotated as ‘Unresolved’ based on similar analysis. Cluster 40 cells expressed macrophages marker gene like Lyz2. Compared to the macrophage cluster (cluster 22), cluster 40 cells expressed higher level of C1qa, C1qb, and C1qc. However, Cluster 40 cells had less UMIs per cells (FIG. 8D) than Cluster 22 cells. Applicants also labeled Cluster 40 as ‘Unresolved’.

Applicants further removed clusters enriched for doublets. To this end, Applicants analyzed PP cells with Scrublet (Wolock et al., 2018), identifying clusters 29, 41, and 43 cells with high doublet scores, together accounting for 0.63% (232 of 36,797) of the PP cells. Cluster 42 cells also had high doublet scores, albeit lower than these other three clusters, and may be further potential doublets.

Clustering LP cells. Applicants used the 35,691 PP cells that were both confidently assigned to clusters in the crossvalidation above and had a k-NN probability greater than 0.5, to train a k-NN classifier (k=11), and used it to classify the 21,270 LP cells. The vast majority of LP cells (97.4%, 20,724/21,270) were assigned to 42 of the 46 clusters with k-NN probability greater than 0.5. Applicants refined the clustering as done for the Peyer's patch data but only for the cells with k-NN probabilities less than 0.5. This reassigned only 0.12% (26/21,270) cells. 34 of the 42 clusters had >15 cells. To find potential LP-specific clusters (cell types that were only observed in LP), Applicants concatenated the data from PPs and LP and then clustered the merged data. Of the clusters enriched in cells from LP (more than three times the number of cells from LP than PPs), two consisted of mast cells and three of plasma B cells. One of these clusters had cells with a low number of UMIs per cell without apparent marker genes. One LP-enriched cluster consisted of fibroblasts that expressed marker genes, such as Col15a1, Ecm1, and Col6a5.

Ten-fold cross validation of the 21,223 LP cells in 34 clusters with >15 cells showed 94.59% accuracy for all cells. Cells from three small clusters 5, 19, and 42 (32, 41, and 19 cells, respectively) had relatively low cross-validation accuracies of 0.41, 0.56, and 0.53, respectively. Overall, ˜93.70% (19,886/21,223) were assigned robustly (correctly classified in crossvalidation and with k-NN probabilities greater than 0.5).

Cellular composition changes. As different gating strategies (IgD^{low} or CD3⁻CD19⁻) directly influenced the frequencies of T and B cells, which subsequently affected the proportions of all other cell types, Applicants separately quantified cell compositions in B cells, T cells, and other non-TB cell types. In addition, for analyzing changes in the composition of B cell subsets, Applicants did not include cells from the experiments with CD3⁻CD19⁻ sorting.

Applicants used the negative binomial regression model with treatment (OVA or PBS) and spatial information (PP or LP) as covariates. The total number of analyzed cells (e.g., the total number of T cells when quantifying T cells variations) from each experiment was used as an offset variable. The P value for the significance of treatment (OVA) on a cell type was assessed using the Wald test on the regression coefficient. Applicants performed similar analyses to quantify cell composition changes between PPs and LP but only using cells in homeostasis. In addition, Applicants used spatial information (PP or LP) as a covariate and the total number of analyzed cells from each experiment as an offset variable. The P value for the significance of location information (LP) on a cell type was also assessed using the Wald test on the regression coefficient.

Cell sampling frequencies. To estimate the number of required cells such that Applicants have the power to recover rare cell types, Applicants used the online tool: http://satijalab.org/howmanycells. The method assumes that the probability of observing at least N cells of a cell type in a sample of size K can be modeled by the cumulative distribution function of a negative binomial NBcdf(K; N, p), where p is the relative abundance of this cell type.

Topic modeling. To help guide LDA to find the informative topics, Applicants learned multiple topic models for each subgroup of cells separately. (Applicants had found that topic modeling of all subsets together mostly identifies cell type programs; data not shown.) Specifically, Applicants used the FitGoM( ) function from the CountClust R package (Dey et al., 2017) to fit LDA topic models to the UMI counts (Bielecki et al., 2018) for cells belonging to each major identified cell type from the LP and PP regions. This resulted in 12 models, two for each of the following cell types: T cells (583 LP cells, 12,187 PP cells), DC cells (3,738 LP cells, 3,530 PP cells), B cells (5,648 LP cells, 7,376 cells), ILC cells (5,484 LP cells, 9,396 PP cells), myeloid cells (1,825 LP cells, 579 PP cells), and stromal cells (90 LP cells, 443 PP cells). Genes starting with ‘Rpl’ or ‘Rps’ were removed from the counts matrix prior to fitting the topic models, leaving a total of 19,108 genes included in each count matrix. The number of topics to fit and the tolerance value are required to run FitGoM( ) function. Thus, for each cell type and region, Applicants fit a range of K and then used compGoM( ) to compute the Bayesian Information Criterion (BIC) the estimated likelihood, from which Applicants calculated the Akaike Information Criterion (AIC). The final choice of K value was primarily guided by the BIC curve; Applicants aimed to choose a K at which the BIC was minimal, or decreasing less quickly. Applicants set the tolerance value to 0.1. The top genes to highlight for each topic were selected using the ExtractTopFeatures( ) function.

Statistical analysis. Mice from which Applicants failed to isolate a sufficient number of live cells for downstream analysis were excluded. Prism 7 (GraphPad Software) was used to perform two-tailed t-test and Fisher's exact test as indicated (except for RNA-seq data). P-values from multiple comparisons were adjusted in R.

Code availability. Code will be made available from bitbucket: bitbucket.org/jerry00/mouse_small_intestine_immune_cell_atlas/src/master/

Data availability. The data are deposited in the Gene Expression Omnibus (GEO; GSE124880, www.ncbi.nlm.nih.gov/geo/query/acc.cgi?acc=GSE124880, enter token ciczsgwmxhkptsl into the box), and the Single Cell Portal portals.broadinstitute.org/single cell/study/fasi-immune-mouse-small-intestine.

TABLE 1
Differential expression analysis between the cells in each cluster and the remaining cells
in PP regions, related to FIG. 1. In certain embodiments, the genes can be used as markers
for the cell types represented by each cluster. The table disclosed here represents only
the genes with adjusted p values of 0 or less than or equal to 3.23E−267.

| | | | percentage of | | | |
|---|---|---|---|---|---|---|
| | | percentage of | expressing cells | | | |
| | cluster | expressiing cells | in the | LN (average | | p value, |
| gene | ID | in the cluster | remaining cells | fold change) | p value | adjusted |
| Igfbp4 | 1 | 0.632 | 0.098 | 1.05 | 0.0E+00 | 0.0E+00 |
| Lef1 | 1 | 0.804 | 0.16 | 0.87 | 0.0E+00 | 0.0E+00 |
| Cd3d | 1 | 0.892 | 0.241 | 0.69 | 0.0E+00 | 0.0E+00 |
| Trac | 1 | 0.803 | 0.197 | 0.68 | 0.0E+00 | 0.0E+00 |
| Ms4a4b | 1 | 0.884 | 0.298 | 0.64 | 0.0E+00 | 0.0E+00 |
| Trbc2 | 1 | 0.962 | 0.484 | 0.63 | 0.0E+00 | 0.0E+00 |
| Cd3g | 1 | 0.907 | 0.259 | 0.60 | 0.0E+00 | 0.0E+00 |
| Cd3e | 1 | 0.846 | 0.219 | 0.60 | 0.0E+00 | 0.0E+00 |
| Ms4a6b | 1 | 0.745 | 0.31 | 0.51 | 0.0E+00 | 0.0E+00 |
| Lat | 1 | 0.755 | 0.27 | 0.51 | 0.0E+00 | 0.0E+00 |
| Dapl1 | 1 | 0.327 | 0.061 | 0.47 | 0.0E+00 | 0.0E+00 |
| Klf2 | 1 | 0.91 | 0.57 | 0.45 | 0.0E+00 | 0.0E+00 |
| Bcl2 | 1 | 0.744 | 0.487 | 0.43 | 0.0E+00 | 0.0E+00 |
| S1pr1 | 1 | 0.469 | 0.113 | 0.39 | 0.0E+00 | 0.0E+00 |
| Atp1b3 | 1 | 0.635 | 0.311 | 0.39 | 0.0E+00 | 0.0E+00 |
| Cd2 | 1 | 0.651 | 0.262 | 0.37 | 0.0E+00 | 0.0E+00 |
| Arl4c | 1 | 0.48 | 0.153 | 0.37 | 0.0E+00 | 0.0E+00 |
| Skap1 | 1 | 0.674 | 0.307 | 0.37 | 0.0E+00 | 0.0E+00 |
| Bcl11b | 1 | 0.456 | 0.119 | 0.35 | 0.0E+00 | 0.0E+00 |
| Cd247 | 1 | 0.488 | 0.14 | 0.34 | 0.0E+00 | 0.0E+00 |
| Ccr7 | 1 | 0.617 | 0.233 | 0.33 | 0.0E+00 | 0.0E+00 |
| Fxyd5 | 1 | 0.9 | 0.633 | 0.33 | 0.0E+00 | 0.0E+00 |
| Satb1 | 1 | 0.622 | 0.33 | 0.33 | 0.0E+00 | 0.0E+00 |
| Pdlim4 | 1 | 0.295 | 0.026 | 0.32 | 0.0E+00 | 0.0E+00 |
| Thy1 | 1 | 0.546 | 0.24 | 0.32 | 0.0E+00 | 0.0E+00 |
| Dgka | 1 | 0.439 | 0.137 | 0.31 | 0.0E+00 | 0.0E+00 |
| Wdr89 | 1 | 0.958 | 0.84 | 0.31 | 0.0E+00 | 0.0E+00 |
| Dusp10 | 1 | 0.36 | 0.167 | 0.31 | 0.0E+00 | 0.0E+00 |
| Actn1 | 1 | 0.419 | 0.109 | 0.31 | 0.0E+00 | 0.0E+00 |
| Cd27 | 1 | 0.632 | 0.308 | 0.30 | 0.0E+00 | 0.0E+00 |
| Ccnd2 | 1 | 0.669 | 0.374 | 0.28 | 0.0E+00 | 0.0E+00 |
| Lck | 1 | 0.762 | 0.441 | 0.28 | 0.0E+00 | 0.0E+00 |
| Pik3ip1 | 1 | 0.416 | 0.152 | 0.28 | 0.0E+00 | 0.0E+00 |
| Ass1 | 1 | 0.394 | 0.108 | 0.28 | 0.0E+00 | 0.0E+00 |
| Cd28 | 1 | 0.464 | 0.184 | 0.27 | 0.0E+00 | 0.0E+00 |
| Npc2 | 1 | 0.878 | 0.57 | 0.27 | 0.0E+00 | 0.0E+00 |
| Cd79a | 2 | 0.995 | 0.186 | 1.37 | 0.0E+00 | 0.0E+00 |
| Iglc2 | 2 | 0.897 | 0.165 | 1.26 | 0.0E+00 | 0.0E+00 |
| Fcmr | 2 | 0.817 | 0.1 | 1.19 | 0.0E+00 | 0.0E+00 |
| Ighd | 2 | 0.738 | 0.028 | 1.13 | 0.0E+00 | 0.0E+00 |
| Ebf1 | 2 | 0.971 | 0.147 | 1.09 | 0.0E+00 | 0.0E+00 |
| Apoe | 2 | 0.618 | 0.196 | 1.05 | 0.0E+00 | 0.0E+00 |
| H2-DMb2 | 2 | 0.966 | 0.234 | 0.97 | 0.0E+00 | 0.0E+00 |
| Cd74 | 2 | 0.998 | 0.634 | 0.92 | 0.0E+00 | 0.0E+00 |
| H2-Aa | 2 | 0.994 | 0.44 | 0.88 | 0.0E+00 | 0.0E+00 |
| Cd79b | 2 | 0.952 | 0.234 | 0.87 | 0.0E+00 | 0.0E+00 |
| Fcer2a | 2 | 0.613 | 0.049 | 0.80 | 0.0E+00 | 0.0E+00 |
| Ighm | 2 | 0.923 | 0.379 | 0.77 | 0.0E+00 | 0.0E+00 |
| H2-Eb1 | 2 | 0.989 | 0.397 | 0.72 | 0.0E+00 | 0.0E+00 |
| Iglc3 | 2 | 0.886 | 0.213 | 0.72 | 0.0E+00 | 0.0E+00 |
| Bank1 | 2 | 0.628 | 0.057 | 0.69 | 0.0E+00 | 0.0E+00 |
| H2-Ab1 | 2 | 0.99 | 0.476 | 0.69 | 0.0E+00 | 0.0E+00 |
| Cd72 | 2 | 0.628 | 0.17 | 0.63 | 0.0E+00 | 0.0E+00 |
| Hvcn1 | 2 | 0.712 | 0.237 | 0.60 | 0.0E+00 | 0.0E+00 |
| Cd83 | 2 | 0.735 | 0.25 | 0.58 | 0.0E+00 | 0.0E+00 |
| Vpreb3 | 2 | 0.635 | 0.111 | 0.58 | 0.0E+00 | 0.0E+00 |
| Tnfrsf13c | 2 | 0.687 | 0.125 | 0.57 | 0.0E+00 | 0.0E+00 |
| Cd37 | 2 | 0.902 | 0.551 | 0.56 | 0.0E+00 | 0.0E+00 |
| Ms4a1 | 2 | 0.876 | 0.142 | 0.56 | 0.0E+00 | 0.0E+00 |
| Mef2c | 2 | 0.811 | 0.249 | 0.56 | 0.0E+00 | 0.0E+00 |
| Snn | 2 | 0.524 | 0.058 | 0.55 | 0.0E+00 | 0.0E+00 |
| Cd52 | 2 | 0.991 | 0.862 | 0.54 | 0.0E+00 | 0.0E+00 |
| Fcrl1 | 2 | 0.493 | 0.077 | 0.51 | 0.0E+00 | 0.0E+00 |
| Capg | 2 | 0.635 | 0.284 | 0.45 | 0.0E+00 | 0.0E+00 |
| Ly6d | 2 | 0.301 | 0.031 | 0.44 | 0.0E+00 | 0.0E+00 |
| Ifi30 | 2 | 0.787 | 0.264 | 0.44 | 0.0E+00 | 0.0E+00 |
| Scd1 | 2 | 0.404 | 0.039 | 0.43 | 0.0E+00 | 0.0E+00 |
| Siglecg | 2 | 0.569 | 0.151 | 0.42 | 0.0E+00 | 0.0E+00 |
| H2-Ob | 2 | 0.542 | 0.131 | 0.41 | 0.0E+00 | 0.0E+00 |
| H2-Oa | 2 | 0.545 | 0.185 | 0.40 | 0.0E+00 | 0.0E+00 |
| Fcrla | 2 | 0.535 | 0.137 | 0.40 | 0.0E+00 | 0.0E+00 |
| Ly6e | 2 | 0.673 | 0.332 | 0.39 | 0.0E+00 | 0.0E+00 |
| Pou2f2 | 2 | 0.578 | 0.204 | 0.39 | 0.0E+00 | 0.0E+00 |
| Cd19 | 2 | 0.496 | 0.098 | 0.38 | 0.0E+00 | 0.0E+00 |
| Sapcd1 | 2 | 0.286 | 0.035 | 0.38 | 0.0E+00 | 0.0E+00 |
| A530040E14Rik | 2 | 0.395 | 0.099 | 0.38 | 0.0E+00 | 0.0E+00 |
| Napsa | 2 | 0.686 | 0.238 | 0.37 | 0.0E+00 | 0.0E+00 |
| H2-DMa | 2 | 0.768 | 0.3 | 0.36 | 0.0E+00 | 0.0E+00 |
| AC125149.3 | 2 | 0.36 | 0.094 | 0.36 | 0.0E+00 | 0.0E+00 |
| Fam43a | 2 | 0.34 | 0.058 | 0.35 | 0.0E+00 | 0.0E+00 |
| Cd55 | 2 | 0.347 | 0.05 | 0.35 | 0.0E+00 | 0.0E+00 |
| Dok3 | 2 | 0.44 | 0.096 | 0.33 | 0.0E+00 | 0.0E+00 |
| Pold4 | 2 | 0.683 | 0.338 | 0.31 | 0.0E+00 | 0.0E+00 |
| Blk | 2 | 0.362 | 0.08 | 0.31 | 0.0E+00 | 0.0E+00 |
| Cxcr4 | 2 | 0.562 | 0.31 | 0.30 | 0.0E+00 | 0.0E+00 |
| Lmo2 | 2 | 0.386 | 0.105 | 0.29 | 0.0E+00 | 0.0E+00 |
| Chchd10 | 2 | 0.549 | 0.213 | 0.28 | 0.0E+00 | 0.0E+00 |
| Ralgps2 | 2 | 0.393 | 0.133 | 0.28 | 0.0E+00 | 0.0E+00 |
| Cxcr5 | 2 | 0.329 | 0.097 | 0.27 | 0.0E+00 | 0.0E+00 |
| Pkig | 2 | 0.611 | 0.225 | 0.27 | 0.0E+00 | 0.0E+00 |
| Serpinb1a | 2 | 0.481 | 0.182 | 0.26 | 0.0E+00 | 0.0E+00 |
| Ms4a4c | 2 | 0.28 | 0.066 | 0.26 | 0.0E+00 | 0.0E+00 |
| Cd22 | 2 | 0.35 | 0.078 | 0.26 | 0.0E+00 | 0.0E+00 |
| Unc93b1 | 2 | 0.593 | 0.246 | 0.25 | 0.0E+00 | 0.0E+00 |
| Ptpn6 | 2 | 0.714 | 0.426 | 0.25 | 0.0E+00 | 0.0E+00 |
| Ptp4a3 | 2 | 0.62 | 0.336 | 0.25 | 0.0E+00 | 0.0E+00 |
| Dusp2 | 2 | 0.616 | 0.354 | 0.34 | 4.4E−299 | 8.4E−295 |
| Foxp1 | 2 | 0.646 | 0.435 | 0.28 | 2.2E−292 | 4.3E−288 |
| Cxcr6 | 3 | 0.935 | 0.166 | 1.27 | 0.0E+00 | 0.0E+00 |
| Car2 | 3 | 0.915 | 0.198 | 1.21 | 0.0E+00 | 0.0E+00 |
| Lgals3 | 3 | 0.938 | 0.232 | 1.17 | 0.0E+00 | 0.0E+00 |
| Tmem176b | 3 | 0.975 | 0.222 | 1.17 | 0.0E+00 | 0.0E+00 |
| Tmem176a | 3 | 0.952 | 0.203 | 1.07 | 0.0E+00 | 0.0E+00 |
| Serpinb1a | 3 | 0.777 | 0.161 | 1.03 | 0.0E+00 | 0.0E+00 |
| AW112010 | 3 | 0.991 | 0.553 | 0.96 | 0.0E+00 | 0.0E+00 |
| Upp1 | 3 | 0.722 | 0.068 | 0.93 | 0.0E+00 | 0.0E+00 |
| Il7r | 3 | 0.911 | 0.348 | 0.89 | 0.0E+00 | 0.0E+00 |
| Sepp1 | 3 | 0.904 | 0.35 | 0.88 | 0.0E+00 | 0.0E+00 |
| Junb | 3 | 0.964 | 0.736 | 0.88 | 0.0E+00 | 0.0E+00 |
| Ffar2 | 3 | 0.693 | 0.091 | 0.84 | 0.0E+00 | 0.0E+00 |
| Itm2b | 3 | 0.999 | 0.83 | 0.83 | 0.0E+00 | 0.0E+00 |
| Id2 | 3 | 0.824 | 0.32 | 0.83 | 0.0E+00 | 0.0E+00 |
| Lmo4 | 3 | 0.86 | 0.286 | 0.83 | 0.0E+00 | 0.0E+00 |
| Klrk1 | 3 | 0.742 | 0.154 | 0.82 | 0.0E+00 | 0.0E+00 |
| Emb | 3 | 0.914 | 0.413 | 0.81 | 0.0E+00 | 0.0E+00 |
| Gadd45b | 3 | 0.621 | 0.27 | 0.81 | 0.0E+00 | 0.0E+00 |
| Tcrg-C1 | 3 | 0.637 | 0.078 | 0.80 | 0.0E+00 | 0.0E+00 |
| Rora | 3 | 0.756 | 0.153 | 0.76 | 0.0E+00 | 0.0E+00 |
| Ncoa7 | 3 | 0.687 | 0.155 | 0.75 | 0.0E+00 | 0.0E+00 |
| Rgs1 | 3 | 0.737 | 0.339 | 0.75 | 0.0E+00 | 0.0E+00 |
| Zfp36l1 | 3 | 0.945 | 0.582 | 0.74 | 0.0E+00 | 0.0E+00 |
| Ncr1 | 3 | 0.709 | 0.116 | 0.69 | 0.0E+00 | 0.0E+00 |
| Ifngr1 | 3 | 0.814 | 0.41 | 0.67 | 0.0E+00 | 0.0E+00 |
| Ckb | 3 | 0.802 | 0.189 | 0.67 | 0.0E+00 | 0.0E+00 |
| Btg2 | 3 | 0.924 | 0.708 | 0.66 | 0.0E+00 | 0.0E+00 |
| Serpina3g | 3 | 0.578 | 0.134 | 0.66 | 0.0E+00 | 0.0E+00 |
| Ikzf2 | 3 | 0.705 | 0.182 | 0.66 | 0.0E+00 | 0.0E+00 |
| Gimap3 | 3 | 0.88 | 0.487 | 0.65 | 0.0E+00 | 0.0E+00 |
| Nfkbia | 3 | 0.856 | 0.554 | 0.62 | 0.0E+00 | 0.0E+00 |
| Hcst | 3 | 0.872 | 0.502 | 0.62 | 0.0E+00 | 0.0E+00 |
| Pim1 | 3 | 0.823 | 0.476 | 0.61 | 0.0E+00 | 0.0E+00 |
| Ctsw | 3 | 0.705 | 0.212 | 0.58 | 0.0E+00 | 0.0E+00 |
| Fcer1g | 3 | 0.904 | 0.324 | 0.58 | 0.0E+00 | 0.0E+00 |
| Cd69 | 3 | 0.726 | 0.391 | 0.56 | 0.0E+00 | 0.0E+00 |
| Stk19 | 3 | 0.567 | 0.125 | 0.56 | 0.0E+00 | 0.0E+00 |
| Bhlhe40 | 3 | 0.688 | 0.266 | 0.56 | 0.0E+00 | 0.0E+00 |
| St6galnac3 | 3 | 0.633 | 0.134 | 0.56 | 0.0E+00 | 0.0E+00 |
| S100a10 | 3 | 0.914 | 0.561 | 0.55 | 0.0E+00 | 0.0E+00 |
| Il18r1 | 3 | 0.592 | 0.116 | 0.55 | 0.0E+00 | 0.0E+00 |
| Podnl1 | 3 | 0.625 | 0.132 | 0.54 | 0.0E+00 | 0.0E+00 |
| Ltb | 3 | 0.958 | 0.812 | 0.53 | 0.0E+00 | 0.0E+00 |
| Chad | 3 | 0.55 | 0.086 | 0.52 | 0.0E+00 | 0.0E+00 |
| Cd82 | 3 | 0.817 | 0.405 | 0.51 | 0.0E+00 | 0.0E+00 |
| Gimap4 | 3 | 0.821 | 0.441 | 0.51 | 0.0E+00 | 0.0E+00 |
| Prr29 | 3 | 0.51 | 0.078 | 0.50 | 0.0E+00 | 0.0E+00 |
| Txk | 3 | 0.747 | 0.338 | 0.50 | 0.0E+00 | 0.0E+00 |
| Sla | 3 | 0.629 | 0.265 | 0.49 | 0.0E+00 | 0.0E+00 |
| Asb2 | 3 | 0.507 | 0.118 | 0.49 | 0.0E+00 | 0.0E+00 |
| Serpinb6b | 3 | 0.461 | 0.138 | 0.48 | 0.0E+00 | 0.0E+00 |
| Maf | 3 | 0.527 | 0.115 | 0.48 | 0.0E+00 | 0.0E+00 |
| Ramp3 | 3 | 0.345 | 0.086 | 0.47 | 0.0E+00 | 0.0E+00 |
| Serpinb9 | 3 | 0.41 | 0.115 | 0.47 | 0.0E+00 | 0.0E+00 |
| Maff | 3 | 0.508 | 0.156 | 0.45 | 0.0E+00 | 0.0E+00 |
| Ahcyl2 | 3 | 0.584 | 0.204 | 0.45 | 0.0E+00 | 0.0E+00 |
| Hk2 | 3 | 0.361 | 0.053 | 0.45 | 0.0E+00 | 0.0E+00 |
| Nrip1 | 3 | 0.652 | 0.303 | 0.44 | 0.0E+00 | 0.0E+00 |
| Icos | 3 | 0.468 | 0.118 | 0.44 | 0.0E+00 | 0.0E+00 |
| Tnfrsf25 | 3 | 0.527 | 0.12 | 0.44 | 0.0E+00 | 0.0E+00 |
| 2010300C02Rik | 3 | 0.455 | 0.073 | 0.43 | 0.0E+00 | 0.0E+00 |
| Cd160 | 3 | 0.503 | 0.103 | 0.42 | 0.0E+00 | 0.0E+00 |
| Rorc | 3 | 0.437 | 0.064 | 0.41 | 0.0E+00 | 0.0E+00 |
| Nrgn | 3 | 0.509 | 0.141 | 0.40 | 0.0E+00 | 0.0E+00 |
| Selplg | 3 | 0.901 | 0.574 | 0.40 | 0.0E+00 | 0.0E+00 |
| Tcrg-C4 | 3 | 0.366 | 0.079 | 0.38 | 0.0E+00 | 0.0E+00 |
| C4b | 3 | 0.288 | 0.026 | 0.38 | 0.0E+00 | 0.0E+00 |
| Ahr | 3 | 0.463 | 0.151 | 0.38 | 0.0E+00 | 0.0E+00 |
| Cxcr3 | 3 | 0.413 | 0.078 | 0.38 | 0.0E+00 | 0.0E+00 |
| Kcnk1 | 3 | 0.397 | 0.048 | 0.37 | 0.0E+00 | 0.0E+00 |
| Slc6a20a | 3 | 0.28 | 0.01 | 0.37 | 0.0E+00 | 0.0E+00 |
| Tspo | 3 | 0.864 | 0.578 | 0.36 | 0.0E+00 | 0.0E+00 |
| F2r | 3 | 0.385 | 0.076 | 0.36 | 0.0E+00 | 0.0E+00 |
| Fgl2 | 3 | 0.385 | 0.1 | 0.35 | 0.0E+00 | 0.0E+00 |
| Prelid2 | 3 | 0.444 | 0.124 | 0.34 | 0.0E+00 | 0.0E+00 |
| Kit | 3 | 0.436 | 0.109 | 0.33 | 0.0E+00 | 0.0E+00 |
| Klrb1b | 3 | 0.313 | 0.04 | 0.32 | 0.0E+00 | 0.0E+00 |
| Aqp3 | 3 | 0.345 | 0.045 | 0.32 | 0.0E+00 | 0.0E+00 |
| Dkkl1 | 3 | 0.36 | 0.071 | 0.31 | 0.0E+00 | 0.0E+00 |
| Lpar6 | 3 | 0.4 | 0.108 | 0.31 | 0.0E+00 | 0.0E+00 |
| St3gal6 | 3 | 0.441 | 0.141 | 0.31 | 0.0E+00 | 0.0E+00 |
| Cd7 | 3 | 0.733 | 0.349 | 0.31 | 0.0E+00 | 0.0E+00 |
| Mdfic | 3 | 0.388 | 0.108 | 0.31 | 0.0E+00 | 0.0E+00 |
| Ppt2 | 3 | 0.359 | 0.081 | 0.31 | 0.0E+00 | 0.0E+00 |
| Klrb1f | 3 | 0.373 | 0.084 | 0.30 | 0.0E+00 | 0.0E+00 |
| Il2ra | 3 | 0.357 | 0.084 | 0.30 | 0.0E+00 | 0.0E+00 |
| Tmem64 | 3 | 0.396 | 0.12 | 0.30 | 0.0E+00 | 0.0E+00 |
| Prrt1 | 3 | 0.337 | 0.051 | 0.30 | 0.0E+00 | 0.0E+00 |
| Cited4 | 3 | 0.408 | 0.11 | 0.30 | 0.0E+00 | 0.0E+00 |
| Eef2k | 3 | 0.371 | 0.082 | 0.30 | 0.0E+00 | 0.0E+00 |
| Actn2 | 3 | 0.25 | 0.028 | 0.29 | 0.0E+00 | 0.0E+00 |
| Lysmd2 | 3 | 0.361 | 0.09 | 0.29 | 0.0E+00 | 0.0E+00 |
| Clnk | 3 | 0.311 | 0.052 | 0.27 | 0.0E+00 | 0.0E+00 |
| Rbpms2 | 3 | 0.267 | 0.016 | 0.27 | 0.0E+00 | 0.0E+00 |
| Gstm5 | 3 | 0.25 | 0.025 | 0.27 | 0.0E+00 | 0.0E+00 |
| Xlr4a | 3 | 0.338 | 0.078 | 0.27 | 0.0E+00 | 0.0E+00 |
| Plekhg1 | 3 | 0.285 | 0.037 | 0.26 | 0.0E+00 | 0.0E+00 |
| Tasp1 | 3 | 0.326 | 0.079 | 0.26 | 0.0E+00 | 0.0E+00 |
| Gjb2 | 3 | 0.237 | 0.023 | 0.26 | 0.0E+00 | 0.0E+00 |
| Trbc1 | 3 | 0.862 | 0.426 | 0.26 | 0.0E+00 | 0.0E+00 |
| Bcl2a1d | 3 | 0.45 | 0.153 | 0.26 | 0.0E+00 | 0.0E+00 |
| Rnase4 | 3 | 0.303 | 0.056 | 0.25 | 0.0E+00 | 0.0E+00 |
| Fhl2 | 3 | 0.291 | 0.055 | 0.25 | 0.0E+00 | 0.0E+00 |
| Acot7 | 3 | 0.471 | 0.177 | 0.29 | 3.4E−313 | 0.0E+00 |
| Il2rb | 3 | 0.514 | 0.195 | 0.30 | 1.1E−311 | 2.3E−307 |
| Gabarapl1 | 3 | 0.318 | 0.082 | 0.25 | 5.9E−311 | 1.1E−306 |
| Shisa5 | 3 | 0.991 | 0.869 | 0.35 | 0.0E+00 | 4.1E−305 |
| Tnf | 3 | 0.272 | 0.052 | 0.31 | 0.0E+00 | 3.0E−304 |
| Il22 | 3 | 0.264 | 0.049 | 1.13 | 3.3E−302 | 6.4E−298 |
| Nabp1 | 3 | 0.569 | 0.255 | 0.46 | 3.5E−298 | 6.7E−294 |
| Gem | 3 | 0.587 | 0.274 | 0.59 | 2.1E−297 | 4.1E−293 |
| Socs2 | 3 | 0.301 | 0.068 | 0.29 | 1.1E−293 | 2.1E−289 |
| Gpr183 | 3 | 0.635 | 0.313 | 0.36 | 5.8E−288 | 1.1E−283 |
| Uhrf2 | 3 | 0.564 | 0.271 | 0.44 | 1.3E−286 | 2.5E−282 |
| Ccdc184 | 3 | 0.27 | 0.054 | 0.35 | 9.5E−286 | 1.8E−281 |
| Wls | 3 | 0.329 | 0.086 | 0.25 | 3.4E−283 | 6.6E−279 |
| Cers4 | 3 | 0.419 | 0.149 | 0.30 | 2.4E−282 | 4.6E−278 |
| Mast4 | 3 | 0.396 | 0.131 | 0.29 | 2.5E−282 | 4.8E−278 |
| Stk24 | 3 | 0.593 | 0.325 | 0.31 | 1.9E−281 | 3.7E−277 |
| Hbegf | 3 | 0.196 | 0.027 | 0.26 | 5.8E−279 | 1.1E−274 |
| Dusp5 | 3 | 0.616 | 0.313 | 0.39 | 4.6E−275 | 8.8E−271 |
| Nr4a1 | 3 | 0.684 | 0.362 | 0.41 | 2.1E−273 | 4.0E−269 |
| Il22 | 4 | 0.358 | 0.044 | 1.64 | 0.0E+00 | 0.0E+00 |
| Tmem176a | 4 | 0.979 | 0.209 | 1.42 | 0.0E+00 | 0.0E+00 |
| Odc1 | 4 | 0.803 | 0.24 | 1.38 | 0.0E+00 | 0.0E+00 |
| S100a4 | 4 | 0.884 | 0.11 | 1.37 | 0.0E+00 | 0.0E+00 |
| Fos | 4 | 0.874 | 0.472 | 1.33 | 0.0E+00 | 0.0E+00 |
| Tmem176b | 4 | 0.976 | 0.229 | 1.26 | 0.0E+00 | 0.0E+00 |
| Gem | 4 | 0.777 | 0.262 | 1.20 | 0.0E+00 | 0.0E+00 |
| Junb | 4 | 0.97 | 0.738 | 1.15 | 0.0E+00 | 0.0E+00 |
| Ckb | 4 | 0.891 | 0.188 | 1.05 | 0.0E+00 | 0.0E+00 |
| Prr29 | 4 | 0.784 | 0.061 | 1.05 | 0.0E+00 | 0.0E+00 |
| Rasl11a | 4 | 0.603 | 0.042 | 1.02 | 0.0E+00 | 0.0E+00 |
| Ncoa7 | 4 | 0.798 | 0.151 | 1.01 | 0.0E+00 | 0.0E+00 |
| Itm2b | 4 | 0.999 | 0.832 | 1.01 | 0.0E+00 | 0.0E+00 |
| Il7r | 4 | 0.951 | 0.351 | 1.01 | 0.0E+00 | 0.0E+00 |
| Fosb | 4 | 0.623 | 0.152 | 0.99 | 0.0E+00 | 0.0E+00 |
| Jun | 4 | 0.795 | 0.38 | 0.95 | 0.0E+00 | 0.0E+00 |
| Sepp1 | 4 | 0.944 | 0.352 | 0.94 | 0.0E+00 | 0.0E+00 |
| Fcer1g | 4 | 0.98 | 0.324 | 0.91 | 0.0E+00 | 0.0E+00 |
| Cd82 | 4 | 0.901 | 0.403 | 0.90 | 0.0E+00 | 0.0E+00 |
| Ltb | 4 | 0.982 | 0.812 | 0.89 | 0.0E+00 | 0.0E+00 |
| Id2 | 4 | 0.854 | 0.323 | 0.89 | 0.0E+00 | 0.0E+00 |
| Cd7 | 4 | 0.876 | 0.342 | 0.88 | 0.0E+00 | 0.0E+00 |
| Chad | 4 | 0.724 | 0.078 | 0.87 | 0.0E+00 | 0.0E+00 |
| Uhrf2 | 4 | 0.715 | 0.262 | 0.87 | 0.0E+00 | 0.0E+00 |
| Cxcr6 | 4 | 0.861 | 0.179 | 0.87 | 0.0E+00 | 0.0E+00 |
| Sdc4 | 4 | 0.748 | 0.168 | 0.86 | 0.0E+00 | 0.0E+00 |
| Cited4 | 4 | 0.614 | 0.097 | 0.81 | 0.0E+00 | 0.0E+00 |
| Hebp1 | 4 | 0.565 | 0.061 | 0.79 | 0.0E+00 | 0.0E+00 |
| Rora | 4 | 0.73 | 0.162 | 0.75 | 0.0E+00 | 0.0E+00 |
| Nrgn | 4 | 0.719 | 0.128 | 0.74 | 0.0E+00 | 0.0E+00 |
| Nr4a1 | 4 | 0.774 | 0.359 | 0.74 | 0.0E+00 | 0.0E+00 |
| Ier2 | 4 | 0.789 | 0.512 | 0.73 | 0.0E+00 | 0.0E+00 |
| Dusp1 | 4 | 0.749 | 0.444 | 0.72 | 0.0E+00 | 0.0E+00 |
| Sla | 4 | 0.697 | 0.263 | 0.71 | 0.0E+00 | 0.0E+00 |
| Fam110a | 4 | 0.55 | 0.084 | 0.70 | 0.0E+00 | 0.0E+00 |
| Maff | 4 | 0.631 | 0.15 | 0.69 | 0.0E+00 | 0.0E+00 |
| Gadd45b | 4 | 0.623 | 0.273 | 0.68 | 0.0E+00 | 0.0E+00 |
| Nfkbia | 4 | 0.914 | 0.552 | 0.68 | 0.0E+00 | 0.0E+00 |
| Car2 | 4 | 0.803 | 0.215 | 0.67 | 0.0E+00 | 0.0E+00 |
| Zfp36l1 | 4 | 0.935 | 0.587 | 0.67 | 0.0E+00 | 0.0E+00 |
| Dhrs3 | 4 | 0.674 | 0.135 | 0.67 | 0.0E+00 | 0.0E+00 |
| Espn | 4 | 0.591 | 0.063 | 0.65 | 0.0E+00 | 0.0E+00 |
| Jund | 4 | 0.94 | 0.813 | 0.65 | 0.0E+00 | 0.0E+00 |
| Ramp1 | 4 | 0.719 | 0.294 | 0.65 | 0.0E+00 | 0.0E+00 |
| Ccr6 | 4 | 0.618 | 0.077 | 0.64 | 0.0E+00 | 0.0E+00 |
| Cd69 | 4 | 0.761 | 0.391 | 0.63 | 0.0E+00 | 0.0E+00 |
| Tnfsf11 | 4 | 0.359 | 0.046 | 0.63 | 0.0E+00 | 0.0E+00 |
| Oser1 | 4 | 0.707 | 0.349 | 0.63 | 0.0E+00 | 0.0E+00 |
| Bhlhe40 | 4 | 0.709 | 0.269 | 0.62 | 0.0E+00 | 0.0E+00 |
| Ccdc184 | 4 | 0.47 | 0.041 | 0.62 | 0.0E+00 | 0.0E+00 |
| Lmna | 4 | 0.393 | 0.072 | 0.62 | 0.0E+00 | 0.0E+00 |
| Rgs2 | 4 | 0.811 | 0.482 | 0.61 | 0.0E+00 | 0.0E+00 |
| Lmo4 | 4 | 0.817 | 0.295 | 0.60 | 0.0E+00 | 0.0E+00 |
| Rorc | 4 | 0.557 | 0.059 | 0.60 | 0.0E+00 | 0.0E+00 |
| Egr1 | 4 | 0.526 | 0.186 | 0.59 | 0.0E+00 | 0.0E+00 |
| Cited2 | 4 | 0.57 | 0.23 | 0.59 | 0.0E+00 | 0.0E+00 |
| Lgals3 | 4 | 0.742 | 0.255 | 0.58 | 0.0E+00 | 0.0E+00 |
| Klf4 | 4 | 0.457 | 0.123 | 0.58 | 0.0E+00 | 0.0E+00 |
| Nrip1 | 4 | 0.715 | 0.302 | 0.57 | 0.0E+00 | 0.0E+00 |
| Ffar2 | 4 | 0.563 | 0.107 | 0.56 | 0.0E+00 | 0.0E+00 |
| Emb | 4 | 0.846 | 0.424 | 0.56 | 0.0E+00 | 0.0E+00 |
| Sfr1 | 4 | 0.779 | 0.346 | 0.56 | 0.0E+00 | 0.0E+00 |
| Cd81 | 4 | 0.65 | 0.232 | 0.56 | 0.0E+00 | 0.0E+00 |
| Tmem59 | 4 | 0.848 | 0.483 | 0.55 | 0.0E+00 | 0.0E+00 |
| Serpina3g | 4 | 0.543 | 0.142 | 0.55 | 0.0E+00 | 0.0E+00 |
| Tnfrsf25 | 4 | 0.607 | 0.118 | 0.54 | 0.0E+00 | 0.0E+00 |
| Podnl1 | 4 | 0.606 | 0.139 | 0.53 | 0.0E+00 | 0.0E+00 |
| Ube2e3 | 4 | 0.589 | 0.179 | 0.53 | 0.0E+00 | 0.0E+00 |
| Ldhb | 4 | 0.466 | 0.092 | 0.53 | 0.0E+00 | 0.0E+00 |
| Gpr132 | 4 | 0.66 | 0.261 | 0.52 | 0.0E+00 | 0.0E+00 |
| Upp1 | 4 | 0.507 | 0.091 | 0.52 | 0.0E+00 | 0.0E+00 |
| Kit | 4 | 0.551 | 0.103 | 0.51 | 0.0E+00 | 0.0E+00 |
| Dgat1 | 4 | 0.504 | 0.116 | 0.51 | 0.0E+00 | 0.0E+00 |
| Arrdc4 | 4 | 0.499 | 0.094 | 0.50 | 0.0E+00 | 0.0E+00 |
| Znrf1 | 4 | 0.632 | 0.219 | 0.49 | 0.0E+00 | 0.0E+00 |
| Slc6a13 | 4 | 0.41 | 0.032 | 0.49 | 0.0E+00 | 0.0E+00 |
| Rabac1 | 4 | 0.889 | 0.614 | 0.48 | 0.0E+00 | 0.0E+00 |
| M1ap | 4 | 0.417 | 0.037 | 0.48 | 0.0E+00 | 0.0E+00 |
| Il18r1 | 4 | 0.548 | 0.125 | 0.47 | 0.0E+00 | 0.0E+00 |
| Arg1 | 4 | 0.265 | 0.038 | 0.47 | 0.0E+00 | 0.0E+00 |
| Acot7 | 4 | 0.573 | 0.172 | 0.46 | 0.0E+00 | 0.0E+00 |
| Tspo | 4 | 0.871 | 0.58 | 0.46 | 0.0E+00 | 0.0E+00 |
| Asb2 | 4 | 0.504 | 0.122 | 0.46 | 0.0E+00 | 0.0E+00 |
| Lamp1 | 4 | 0.806 | 0.463 | 0.46 | 0.0E+00 | 0.0E+00 |
| Ostf1 | 4 | 0.844 | 0.573 | 0.44 | 0.0E+00 | 0.0E+00 |
| 2010300C02Rik | 4 | 0.456 | 0.077 | 0.44 | 0.0E+00 | 0.0E+00 |
| Fdx1 | 4 | 0.479 | 0.138 | 0.44 | 0.0E+00 | 0.0E+00 |
| Mast4 | 4 | 0.493 | 0.126 | 0.44 | 0.0E+00 | 0.0E+00 |
| Serpinb1a | 4 | 0.606 | 0.181 | 0.43 | 0.0E+00 | 0.0E+00 |
| Kcnk1 | 4 | 0.435 | 0.049 | 0.42 | 0.0E+00 | 0.0E+00 |
| Shisa5 | 4 | 0.994 | 0.87 | 0.42 | 0.0E+00 | 0.0E+00 |
| Pxdc1 | 4 | 0.379 | 0.089 | 0.41 | 0.0E+00 | 0.0E+00 |
| Anxa5 | 4 | 0.609 | 0.22 | 0.41 | 0.0E+00 | 0.0E+00 |
| Sec11c | 4 | 0.862 | 0.516 | 0.41 | 0.0E+00 | 0.0E+00 |
| Tm9sf2 | 4 | 0.484 | 0.149 | 0.40 | 0.0E+00 | 0.0E+00 |
| Itm2c | 4 | 0.733 | 0.362 | 0.40 | 0.0E+00 | 0.0E+00 |
| St6galnac3 | 4 | 0.529 | 0.147 | 0.40 | 0.0E+00 | 0.0E+00 |
| Cmc2 | 4 | 0.562 | 0.194 | 0.39 | 0.0E+00 | 0.0E+00 |
| Dkkl1 | 4 | 0.408 | 0.07 | 0.38 | 0.0E+00 | 0.0E+00 |
| Nrp1 | 4 | 0.354 | 0.042 | 0.37 | 0.0E+00 | 0.0E+00 |
| Ptges | 4 | 0.319 | 0.009 | 0.36 | 0.0E+00 | 0.0E+00 |
| Xlr4a | 4 | 0.399 | 0.076 | 0.36 | 0.0E+00 | 0.0E+00 |
| Il17re | 4 | 0.36 | 0.028 | 0.36 | 0.0E+00 | 0.0E+00 |
| Il2ra | 4 | 0.39 | 0.085 | 0.35 | 0.0E+00 | 0.0E+00 |
| S100a6 | 4 | 0.553 | 0.176 | 0.35 | 0.0E+00 | 0.0E+00 |
| Stom | 4 | 0.358 | 0.034 | 0.35 | 0.0E+00 | 0.0E+00 |
| Piwil4 | 4 | 0.291 | 0.005 | 0.35 | 0.0E+00 | 0.0E+00 |
| Scn1b | 4 | 0.347 | 0.039 | 0.35 | 0.0E+00 | 0.0E+00 |
| Nmrk1 | 4 | 0.339 | 0.086 | 0.35 | 0.0E+00 | 0.0E+00 |
| Igfbp7 | 4 | 0.515 | 0.05 | 0.34 | 0.0E+00 | 0.0E+00 |
| Slc41a3 | 4 | 0.33 | 0.031 | 0.34 | 0.0E+00 | 0.0E+00 |
| Xlr4c | 4 | 0.415 | 0.11 | 0.34 | 0.0E+00 | 0.0E+00 |
| Eef2k | 4 | 0.393 | 0.084 | 0.33 | 0.0E+00 | 0.0E+00 |
| Cntn1 | 4 | 0.312 | 0.014 | 0.33 | 0.0E+00 | 0.0E+00 |
| Xist | 4 | 0.999 | 0.871 | 0.33 | 0.0E+00 | 0.0E+00 |
| Casp4 | 4 | 0.334 | 0.069 | 0.32 | 0.0E+00 | 0.0E+00 |
| Dtx4 | 4 | 0.356 | 0.064 | 0.32 | 0.0E+00 | 0.0E+00 |
| Hmgn3 | 4 | 0.48 | 0.16 | 0.31 | 0.0E+00 | 0.0E+00 |
| Cx3cl1 | 4 | 0.267 | 0.012 | 0.31 | 0.0E+00 | 0.0E+00 |
| Oaz1 | 4 | 0.999 | 0.945 | 0.30 | 0.0E+00 | 0.0E+00 |
| Dkk3 | 4 | 0.309 | 0.033 | 0.30 | 0.0E+00 | 0.0E+00 |
| Serinc2 | 4 | 0.309 | 0.025 | 0.29 | 0.0E+00 | 0.0E+00 |
| Ret | 4 | 0.298 | 0.032 | 0.29 | 0.0E+00 | 0.0E+00 |
| Sdc1 | 4 | 0.302 | 0.048 | 0.29 | 0.0E+00 | 0.0E+00 |
| Igf1r | 4 | 0.31 | 0.053 | 0.27 | 0.0E+00 | 0.0E+00 |
| Clnk | 4 | 0.318 | 0.054 | 0.27 | 0.0E+00 | 0.0E+00 |
| Ddc | 4 | 0.222 | 0.022 | 0.27 | 0.0E+00 | 0.0E+00 |
| Cdc14a | 4 | 0.249 | 0.036 | 0.27 | 0.0E+00 | 0.0E+00 |
| Ptger2 | 4 | 0.252 | 0.03 | 0.26 | 0.0E+00 | 0.0E+00 |
| Vipr2 | 4 | 0.261 | 0.014 | 0.26 | 0.0E+00 | 0.0E+00 |
| Il1r1 | 4 | 0.259 | 0.022 | 0.25 | 0.0E+00 | 0.0E+00 |
| Bcl2 | 4 | 0.85 | 0.511 | 0.41 | 2.2E−302 | 4.2E−298 |
| S100a1 | 4 | 0.291 | 0.058 | 0.26 | 2.8E−300 | 5.3E−296 |
| Aqp3 | 4 | 0.29 | 0.052 | 0.26 | 1.1E−299 | 2.2E−295 |
| Ecm1 | 4 | 0.272 | 0.047 | 0.29 | 5.6E−298 | 1.1E−293 |
| Mrfap1 | 4 | 0.789 | 0.534 | 0.36 | 2.9E−297 | 5.5E−293 |
| Dbp | 4 | 0.306 | 0.076 | 0.31 | 1.4E−289 | 2.8E−285 |
| Rabgap1l | 4 | 0.607 | 0.294 | 0.36 | 3.5E−287 | 6.7E−283 |
| Gpx4 | 4 | 0.903 | 0.657 | 0.38 | 8.7E−286 | 1.7E−281 |
| Agtrap | 4 | 0.362 | 0.1 | 0.27 | 2.2E−282 | 4.2E−278 |
| Tgif1 | 4 | 0.558 | 0.247 | 0.41 | 1.3E−280 | 2.6E−276 |
| Fhl2 | 4 | 0.289 | 0.058 | 0.27 | 2.9E−279 | 5.6E−275 |
| Nfatc2 | 4 | 0.31 | 0.076 | 0.26 | 9.3E−278 | 1.8E−273 |
| Lta | 4 | 0.264 | 0.06 | 0.39 | 3.4E−276 | 6.6E−272 |
| Sptssa | 4 | 0.786 | 0.468 | 0.38 | 1.2E−274 | 2.4E−270 |
| Ptms | 4 | 0.618 | 0.285 | 0.30 | 6.8E−274 | 1.3E−269 |
| Cd8b1 | 5 | 0.947 | 0.092 | 1.29 | 0.0E+00 | 0.0E+00 |
| Cd8a | 5 | 0.799 | 0.06 | 0.90 | 0.0E+00 | 0.0E+00 |
| Dapl1 | 5 | 0.55 | 0.082 | 0.86 | 0.0E+00 | 0.0E+00 |
| Ms4a4b | 5 | 0.935 | 0.373 | 0.76 | 0.0E+00 | 0.0E+00 |
| Cd3d | 5 | 0.934 | 0.326 | 0.75 | 0.0E+00 | 0.0E+00 |
| Cd3e | 5 | 0.912 | 0.299 | 0.69 | 0.0E+00 | 0.0E+00 |
| Thy1 | 5 | 0.793 | 0.265 | 0.67 | 0.0E+00 | 0.0E+00 |
| Lef1 | 5 | 0.818 | 0.246 | 0.67 | 0.0E+00 | 0.0E+00 |
| Cd3g | 5 | 0.942 | 0.344 | 0.62 | 0.0E+00 | 0.0E+00 |
| Trbc2 | 5 | 0.958 | 0.549 | 0.56 | 0.0E+00 | 0.0E+00 |
| Trac | 5 | 0.796 | 0.279 | 0.54 | 0.0E+00 | 0.0E+00 |
| Wdr89 | 5 | 0.98 | 0.854 | 0.52 | 0.0E+00 | 0.0E+00 |
| Lat | 5 | 0.797 | 0.332 | 0.51 | 0.0E+00 | 0.0E+00 |
| Arl4c | 5 | 0.626 | 0.188 | 0.50 | 0.0E+00 | 0.0E+00 |
| Nkg7 | 5 | 0.818 | 0.215 | 0.50 | 0.0E+00 | 0.0E+00 |
| Ms4a6b | 5 | 0.799 | 0.365 | 0.50 | 0.0E+00 | 0.0E+00 |
| Klk8 | 5 | 0.572 | 0.162 | 0.47 | 0.0E+00 | 0.0E+00 |
| Tmsb10 | 5 | 1 | 0.977 | 0.41 | 0.0E+00 | 0.0E+00 |
| Acp5 | 5 | 0.595 | 0.246 | 0.38 | 0.0E+00 | 0.0E+00 |
| Npc2 | 5 | 0.909 | 0.61 | 0.31 | 0.0E+00 | 0.0E+00 |
| Saraf | 5 | 0.76 | 0.431 | 0.39 | 5.5E−314 | 0.0E+00 |
| Igfbp4 | 5 | 0.555 | 0.175 | 0.43 | 1.1E−313 | 0.0E+00 |
| Ccr7 | 5 | 0.678 | 0.28 | 0.38 | 9.7E−298 | 1.9E−293 |
| Klf2 | 5 | 0.931 | 0.614 | 0.40 | 1.4E−276 | 2.6E−272 |
| Bst2 | 6 | 0.998 | 0.243 | 2.99 | 0.0E+00 | 0.0E+00 |
| Ly6c2 | 6 | 0.997 | 0.082 | 2.81 | 0.0E+00 | 0.0E+00 |
| Tyrobp | 6 | 0.999 | 0.263 | 2.67 | 0.0E+00 | 0.0E+00 |
| Siglech | 6 | 0.998 | 0.053 | 2.67 | 0.0E+00 | 0.0E+00 |
| Ccl4 | 6 | 0.868 | 0.134 | 2.60 | 0.0E+00 | 0.0E+00 |
| Klk1 | 6 | 0.96 | 0.041 | 2.60 | 0.0E+00 | 0.0E+00 |
| Plac8 | 6 | 0.999 | 0.204 | 2.59 | 0.0E+00 | 0.0E+00 |
| Cox6a2 | 6 | 0.969 | 0.019 | 2.42 | 0.0E+00 | 0.0E+00 |
| Ly6c1 | 6 | 0.991 | 0.062 | 2.36 | 0.0E+00 | 0.0E+00 |
| Irf8 | 6 | 0.997 | 0.371 | 2.27 | 0.0E+00 | 0.0E+00 |
| Ctsb | 6 | 0.994 | 0.335 | 2.20 | 0.0E+00 | 0.0E+00 |
| Psap | 6 | 0.998 | 0.502 | 2.19 | 0.0E+00 | 0.0E+00 |
| Rnase6 | 6 | 0.992 | 0.124 | 2.15 | 0.0E+00 | 0.0E+00 |
| Grn | 6 | 0.986 | 0.119 | 2.11 | 0.0E+00 | 0.0E+00 |
| Ctsl | 6 | 0.96 | 0.079 | 2.01 | 0.0E+00 | 0.0E+00 |
| Mpeg1 | 6 | 0.983 | 0.048 | 1.97 | 0.0E+00 | 0.0E+00 |
| D13Ertd608e | 6 | 0.924 | 0.007 | 1.92 | 0.0E+00 | 0.0E+00 |
| Klk1b27 | 6 | 0.965 | 0.018 | 1.88 | 0.0E+00 | 0.0E+00 |
| Fth1 | 6 | 1 | 0.978 | 1.80 | 0.0E+00 | 0.0E+00 |
| Pld4 | 6 | 0.975 | 0.098 | 1.78 | 0.0E+00 | 0.0E+00 |
| Ctsh | 6 | 0.988 | 0.27 | 1.76 | 0.0E+00 | 0.0E+00 |
| Ccr9 | 6 | 0.956 | 0.11 | 1.75 | 0.0E+00 | 0.0E+00 |
| Cybb | 6 | 0.978 | 0.146 | 1.70 | 0.0E+00 | 0.0E+00 |
| Lgals1 | 6 | 0.978 | 0.292 | 1.68 | 0.0E+00 | 0.0E+00 |
| Iglc3 | 6 | 0.968 | 0.236 | 1.67 | 0.0E+00 | 0.0E+00 |
| Tcf4 | 6 | 0.977 | 0.184 | 1.67 | 0.0E+00 | 0.0E+00 |
| Dnajc7 | 6 | 0.982 | 0.321 | 1.64 | 0.0E+00 | 0.0E+00 |
| Clec12a | 6 | 0.93 | 0.053 | 1.57 | 0.0E+00 | 0.0E+00 |
| Lair1 | 6 | 0.937 | 0.034 | 1.54 | 0.0E+00 | 0.0E+00 |
| Tsc22d1 | 6 | 0.908 | 0.088 | 1.50 | 0.0E+00 | 0.0E+00 |
| Sell | 6 | 0.973 | 0.28 | 1.49 | 0.0E+00 | 0.0E+00 |
| Cd8b1 | 6 | 0.593 | 0.116 | 1.48 | 0.0E+00 | 0.0E+00 |
| Slpi | 6 | 0.789 | 0.06 | 1.47 | 0.0E+00 | 0.0E+00 |
| Pgls | 6 | 0.979 | 0.41 | 1.45 | 0.0E+00 | 0.0E+00 |
| Tspan13 | 6 | 0.982 | 0.462 | 1.42 | 0.0E+00 | 0.0E+00 |
| St8sia4 | 6 | 0.927 | 0.14 | 1.39 | 0.0E+00 | 0.0E+00 |
| Snx5 | 6 | 0.974 | 0.39 | 1.39 | 0.0E+00 | 0.0E+00 |
| Alox5ap | 6 | 0.937 | 0.052 | 1.39 | 0.0E+00 | 0.0E+00 |
| Ifi27l2a | 6 | 0.844 | 0.368 | 1.37 | 0.0E+00 | 0.0E+00 |
| P2ry14 | 6 | 0.874 | 0.029 | 1.35 | 0.0E+00 | 0.0E+00 |
| Ly6e | 6 | 0.891 | 0.331 | 1.32 | 0.0E+00 | 0.0E+00 |
| Atp1b1 | 6 | 0.897 | 0.137 | 1.31 | 0.0E+00 | 0.0E+00 |
| Bcl11a | 6 | 0.889 | 0.103 | 1.30 | 0.0E+00 | 0.0E+00 |
| Smim5 | 6 | 0.781 | 0.013 | 1.30 | 0.0E+00 | 0.0E+00 |
| Gpx1 | 6 | 0.994 | 0.56 | 1.29 | 0.0E+00 | 0.0E+00 |
| Serp1 | 6 | 0.989 | 0.518 | 1.29 | 0.0E+00 | 0.0E+00 |
| Cd209d | 6 | 0.564 | 0.009 | 1.28 | 0.0E+00 | 0.0E+00 |
| Rilpl2 | 6 | 0.857 | 0.182 | 1.28 | 0.0E+00 | 0.0E+00 |
| Tagln2 | 6 | 0.982 | 0.598 | 1.27 | 0.0E+00 | 0.0E+00 |
| Upb1 | 6 | 0.86 | 0.017 | 1.27 | 0.0E+00 | 0.0E+00 |
| Pltp | 6 | 0.842 | 0.042 | 1.26 | 0.0E+00 | 0.0E+00 |
| Ctss | 6 | 0.96 | 0.357 | 1.26 | 0.0E+00 | 0.0E+00 |
| Ncf1 | 6 | 0.861 | 0.131 | 1.25 | 0.0E+00 | 0.0E+00 |
| Cd47 | 6 | 0.981 | 0.56 | 1.24 | 0.0E+00 | 0.0E+00 |
| Gpr171 | 6 | 0.847 | 0.242 | 1.23 | 0.0E+00 | 0.0E+00 |
| Ctsz | 6 | 0.954 | 0.316 | 1.23 | 0.0E+00 | 0.0E+00 |
| Cd209a | 6 | 0.578 | 0.013 | 1.22 | 0.0E+00 | 0.0E+00 |
| Nucb2 | 6 | 0.868 | 0.035 | 1.22 | 0.0E+00 | 0.0E+00 |
| Unc93b1 | 6 | 0.934 | 0.238 | 1.20 | 0.0E+00 | 0.0E+00 |
| Xbp1 | 6 | 0.906 | 0.229 | 1.20 | 0.0E+00 | 0.0E+00 |
| Dap | 6 | 0.95 | 0.304 | 1.18 | 0.0E+00 | 0.0E+00 |
| Ppfia4 | 6 | 0.864 | 0.034 | 1.18 | 0.0E+00 | 0.0E+00 |
| Rnaset2a | 6 | 0.974 | 0.54 | 1.17 | 0.0E+00 | 0.0E+00 |
| Tmed3 | 6 | 0.896 | 0.159 | 1.16 | 0.0E+00 | 0.0E+00 |
| Syngr2 | 6 | 0.933 | 0.328 | 1.15 | 0.0E+00 | 0.0E+00 |
| Cadm1 | 6 | 0.848 | 0.02 | 1.14 | 0.0E+00 | 0.0E+00 |
| Runx2 | 6 | 0.811 | 0.053 | 1.13 | 0.0E+00 | 0.0E+00 |
| Ccr2 | 6 | 0.777 | 0.066 | 1.12 | 0.0E+00 | 0.0E+00 |
| Gnas | 6 | 0.992 | 0.818 | 1.12 | 0.0E+00 | 0.0E+00 |
| Selplg | 6 | 0.983 | 0.576 | 1.12 | 0.0E+00 | 0.0E+00 |
| Ifnar2 | 6 | 0.868 | 0.126 | 1.11 | 0.0E+00 | 0.0E+00 |
| Cd68 | 6 | 0.83 | 0.039 | 1.11 | 0.0E+00 | 0.0E+00 |
| Prkca | 6 | 0.876 | 0.282 | 1.11 | 0.0E+00 | 0.0E+00 |
| Npc2 | 6 | 0.98 | 0.605 | 1.11 | 0.0E+00 | 0.0E+00 |
| Emp3 | 6 | 0.897 | 0.307 | 1.08 | 0.0E+00 | 0.0E+00 |
| Fcrla | 6 | 0.788 | 0.136 | 1.06 | 0.0E+00 | 0.0E+00 |
| Stx7 | 6 | 0.897 | 0.213 | 1.05 | 0.0E+00 | 0.0E+00 |
| Pkib | 6 | 0.782 | 0.067 | 1.03 | 0.0E+00 | 0.0E+00 |
| Neat1 | 6 | 0.848 | 0.362 | 1.02 | 0.0E+00 | 0.0E+00 |
| Fam174a | 6 | 0.85 | 0.187 | 1.02 | 0.0E+00 | 0.0E+00 |
| Cd7 | 6 | 0.954 | 0.343 | 1.02 | 0.0E+00 | 0.0E+00 |
| Lsp1 | 6 | 0.976 | 0.665 | 1.01 | 0.0E+00 | 0.0E+00 |
| Fyb | 6 | 0.944 | 0.401 | 1.01 | 0.0E+00 | 0.0E+00 |
| Mef2c | 6 | 0.947 | 0.263 | 1.01 | 0.0E+00 | 0.0E+00 |
| Sub1 | 6 | 0.984 | 0.74 | 1.01 | 0.0E+00 | 0.0E+00 |
| S100a6 | 6 | 0.895 | 0.158 | 1.00 | 0.0E+00 | 0.0E+00 |
| Kctd12 | 6 | 0.81 | 0.113 | 1.00 | 0.0E+00 | 0.0E+00 |
| Snx18 | 6 | 0.81 | 0.128 | 1.00 | 0.0E+00 | 0.0E+00 |
| Pir | 6 | 0.67 | 0.008 | 0.99 | 0.0E+00 | 0.0E+00 |
| Trib1 | 6 | 0.722 | 0.066 | 0.98 | 0.0E+00 | 0.0E+00 |
| Irf7 | 6 | 0.712 | 0.12 | 0.98 | 0.0E+00 | 0.0E+00 |
| Kmo | 6 | 0.79 | 0.042 | 0.98 | 0.0E+00 | 0.0E+00 |
| Sec61b | 6 | 0.985 | 0.67 | 0.97 | 0.0E+00 | 0.0E+00 |
| Cst3 | 6 | 0.993 | 0.557 | 0.97 | 0.0E+00 | 0.0E+00 |
| Ier5 | 6 | 0.919 | 0.474 | 0.97 | 0.0E+00 | 0.0E+00 |
| Tubgcp5 | 6 | 0.744 | 0.043 | 0.97 | 0.0E+00 | 0.0E+00 |
| Mbnl1 | 6 | 0.97 | 0.57 | 0.97 | 0.0E+00 | 0.0E+00 |
| Abhd17b | 6 | 0.78 | 0.158 | 0.97 | 0.0E+00 | 0.0E+00 |
| Gltp | 6 | 0.917 | 0.335 | 0.96 | 0.0E+00 | 0.0E+00 |
| Spib | 6 | 0.823 | 0.149 | 0.96 | 0.0E+00 | 0.0E+00 |
| Sec61g | 6 | 0.986 | 0.745 | 0.95 | 0.0E+00 | 0.0E+00 |
| Amica1 | 6 | 0.791 | 0.117 | 0.95 | 0.0E+00 | 0.0E+00 |
| Evi2a | 6 | 0.768 | 0.135 | 0.95 | 0.0E+00 | 0.0E+00 |
| Sh3bgr | 6 | 0.7 | 0.008 | 0.95 | 0.0E+00 | 0.0E+00 |
| Pacsin1 | 6 | 0.819 | 0.133 | 0.94 | 0.0E+00 | 0.0E+00 |
| Lefty1 | 6 | 0.739 | 0.041 | 0.94 | 0.0E+00 | 0.0E+00 |
| Ramp1 | 6 | 0.879 | 0.289 | 0.92 | 0.0E+00 | 0.0E+00 |
| Plaur | 6 | 0.561 | 0.064 | 0.91 | 0.0E+00 | 0.0E+00 |
| Gm2a | 6 | 0.91 | 0.303 | 0.91 | 0.0E+00 | 0.0E+00 |
| H13 | 6 | 0.864 | 0.267 | 0.90 | 0.0E+00 | 0.0E+00 |
| Zeb2 | 6 | 0.725 | 0.072 | 0.89 | 0.0E+00 | 0.0E+00 |
| Cyth4 | 6 | 0.803 | 0.174 | 0.89 | 0.0E+00 | 0.0E+00 |
| Hpse | 6 | 0.73 | 0.079 | 0.89 | 0.0E+00 | 0.0E+00 |
| Gapt | 6 | 0.69 | 0.022 | 0.88 | 0.0E+00 | 0.0E+00 |
| Reep5 | 6 | 0.918 | 0.409 | 0.88 | 0.0E+00 | 0.0E+00 |
| Gsn | 6 | 0.819 | 0.152 | 0.88 | 0.0E+00 | 0.0E+00 |
| Rrbp1 | 6 | 0.798 | 0.202 | 0.87 | 0.0E+00 | 0.0E+00 |
| Blnk | 6 | 0.812 | 0.153 | 0.87 | 0.0E+00 | 0.0E+00 |
| Hsp90b1 | 6 | 0.885 | 0.331 | 0.86 | 0.0E+00 | 0.0E+00 |
| Ech1 | 6 | 0.843 | 0.285 | 0.86 | 0.0E+00 | 0.0E+00 |
| H2-DMb1 | 6 | 0.711 | 0.133 | 0.86 | 0.0E+00 | 0.0E+00 |
| Svbp | 6 | 0.75 | 0.225 | 0.85 | 0.0E+00 | 0.0E+00 |
| Ifnar1 | 6 | 0.758 | 0.199 | 0.84 | 0.0E+00 | 0.0E+00 |
| Manf | 6 | 0.913 | 0.432 | 0.84 | 0.0E+00 | 0.0E+00 |
| Fyn | 6 | 0.777 | 0.221 | 0.84 | 0.0E+00 | 0.0E+00 |
| Tnfrsf13b | 6 | 0.786 | 0.166 | 0.84 | 0.0E+00 | 0.0E+00 |
| Ly86 | 6 | 0.733 | 0.109 | 0.83 | 0.0E+00 | 0.0E+00 |
| Serinc3 | 6 | 0.867 | 0.317 | 0.83 | 0.0E+00 | 0.0E+00 |
| Cd164 | 6 | 0.838 | 0.271 | 0.82 | 0.0E+00 | 0.0E+00 |
| Tmem229b | 6 | 0.706 | 0.069 | 0.82 | 0.0E+00 | 0.0E+00 |
| Stk17b | 6 | 0.859 | 0.565 | 0.82 | 0.0E+00 | 0.0E+00 |
| Mtdh | 6 | 0.863 | 0.382 | 0.81 | 0.0E+00 | 0.0E+00 |
| Irf2bp2 | 6 | 0.712 | 0.106 | 0.81 | 0.0E+00 | 0.0E+00 |
| Atp2a1 | 6 | 0.654 | 0.007 | 0.81 | 0.0E+00 | 0.0E+00 |
| Slamf9 | 6 | 0.592 | 0.01 | 0.79 | 0.0E+00 | 0.0E+00 |
| Ighm | 6 | 0.993 | 0.397 | 0.77 | 0.0E+00 | 0.0E+00 |
| Vimp | 6 | 0.814 | 0.3 | 0.77 | 0.0E+00 | 0.0E+00 |
| Klra17 | 6 | 0.57 | 0.007 | 0.76 | 0.0E+00 | 0.0E+00 |
| I830077J02Rik | 6 | 0.63 | 0.029 | 0.76 | 0.0E+00 | 0.0E+00 |
| Fgfr1op2 | 6 | 0.822 | 0.264 | 0.76 | 0.0E+00 | 0.0E+00 |
| Scimp | 6 | 0.785 | 0.133 | 0.76 | 0.0E+00 | 0.0E+00 |
| Ctsa | 6 | 0.75 | 0.21 | 0.74 | 0.0E+00 | 0.0E+00 |
| R3hdm4 | 6 | 0.798 | 0.291 | 0.74 | 0.0E+00 | 0.0E+00 |
| Abhd17a | 6 | 0.804 | 0.27 | 0.74 | 0.0E+00 | 0.0E+00 |
| Smc6 | 6 | 0.845 | 0.457 | 0.73 | 0.0E+00 | 0.0E+00 |
| Sla2 | 6 | 0.67 | 0.117 | 0.73 | 0.0E+00 | 0.0E+00 |
| Ahnak | 6 | 0.688 | 0.147 | 0.73 | 0.0E+00 | 0.0E+00 |
| Litaf | 6 | 0.728 | 0.186 | 0.72 | 0.0E+00 | 0.0E+00 |
| Timp2 | 6 | 0.68 | 0.132 | 0.72 | 0.0E+00 | 0.0E+00 |
| Gpcpd1 | 6 | 0.773 | 0.264 | 0.71 | 0.0E+00 | 0.0E+00 |
| Edem2 | 6 | 0.687 | 0.145 | 0.71 | 0.0E+00 | 0.0E+00 |
| Plekhm3 | 6 | 0.635 | 0.05 | 0.70 | 0.0E+00 | 0.0E+00 |
| Slc44a2 | 6 | 0.713 | 0.154 | 0.70 | 0.0E+00 | 0.0E+00 |
| Rnf149 | 6 | 0.601 | 0.122 | 0.70 | 0.0E+00 | 0.0E+00 |
| Cdkn2d | 6 | 0.703 | 0.203 | 0.69 | 0.0E+00 | 0.0E+00 |
| Irf1 | 6 | 0.65 | 0.177 | 0.69 | 0.0E+00 | 0.0E+00 |
| Rell1 | 6 | 0.586 | 0.052 | 0.68 | 0.0E+00 | 0.0E+00 |
| Klk4 | 6 | 0.257 | 0.008 | 0.68 | 0.0E+00 | 0.0E+00 |
| Fgr | 6 | 0.623 | 0.049 | 0.68 | 0.0E+00 | 0.0E+00 |
| Clec10a | 6 | 0.48 | 0.007 | 0.68 | 0.0E+00 | 0.0E+00 |
| Tbc1d8 | 6 | 0.593 | 0.025 | 0.67 | 0.0E+00 | 0.0E+00 |
| Trim30a | 6 | 0.597 | 0.087 | 0.66 | 0.0E+00 | 0.0E+00 |
| Spns3 | 6 | 0.564 | 0.028 | 0.64 | 0.0E+00 | 0.0E+00 |
| Lifr | 6 | 0.543 | 0.012 | 0.64 | 0.0E+00 | 0.0E+00 |
| Cd180 | 6 | 0.627 | 0.103 | 0.63 | 0.0E+00 | 0.0E+00 |
| Rpgrip1 | 6 | 0.5 | 0.034 | 0.62 | 0.0E+00 | 0.0E+00 |
| Csf2rb | 6 | 0.577 | 0.039 | 0.62 | 0.0E+00 | 0.0E+00 |
| Lag3 | 6 | 0.548 | 0.06 | 0.62 | 0.0E+00 | 0.0E+00 |
| Tcf12 | 6 | 0.633 | 0.146 | 0.62 | 0.0E+00 | 0.0E+00 |
| Rnf13 | 6 | 0.633 | 0.127 | 0.62 | 0.0E+00 | 0.0E+00 |
| Ctse | 6 | 0.673 | 0.213 | 0.61 | 0.0E+00 | 0.0E+00 |
| Fam3c | 6 | 0.678 | 0.188 | 0.60 | 0.0E+00 | 0.0E+00 |
| Lpgat1 | 6 | 0.511 | 0.063 | 0.60 | 0.0E+00 | 0.0E+00 |
| Rnf122 | 6 | 0.525 | 0.049 | 0.59 | 0.0E+00 | 0.0E+00 |
| Sdc4 | 6 | 0.676 | 0.18 | 0.59 | 0.0E+00 | 0.0E+00 |
| Klrd1 | 6 | 0.685 | 0.139 | 0.59 | 0.0E+00 | 0.0E+00 |
| Havcr1 | 6 | 0.568 | 0.042 | 0.58 | 0.0E+00 | 0.0E+00 |
| Nek6 | 6 | 0.518 | 0.025 | 0.58 | 0.0E+00 | 0.0E+00 |
| Fcer1g | 6 | 0.917 | 0.336 | 0.58 | 0.0E+00 | 0.0E+00 |
| Card11 | 6 | 0.607 | 0.121 | 0.58 | 0.0E+00 | 0.0E+00 |
| Hhex | 6 | 0.553 | 0.093 | 0.58 | 0.0E+00 | 0.0E+00 |
| Arhgef6 | 6 | 0.62 | 0.123 | 0.58 | 0.0E+00 | 0.0E+00 |
| Mef2a | 6 | 0.584 | 0.11 | 0.57 | 0.0E+00 | 0.0E+00 |
| Rasgrp2 | 6 | 0.695 | 0.261 | 0.57 | 0.0E+00 | 0.0E+00 |
| Sema4b | 6 | 0.492 | 0.069 | 0.56 | 0.0E+00 | 0.0E+00 |
| Mctp2 | 6 | 0.5 | 0.025 | 0.56 | 0.0E+00 | 0.0E+00 |
| Csf2rb2 | 6 | 0.49 | 0.016 | 0.56 | 0.0E+00 | 0.0E+00 |
| Paqr5 | 6 | 0.487 | 0.008 | 0.56 | 0.0E+00 | 0.0E+00 |
| Rab33b | 6 | 0.517 | 0.061 | 0.56 | 0.0E+00 | 0.0E+00 |
| Hs3st1 | 6 | 0.551 | 0.071 | 0.55 | 0.0E+00 | 0.0E+00 |
| Scpep1 | 6 | 0.564 | 0.094 | 0.55 | 0.0E+00 | 0.0E+00 |
| Gpr137b | 6 | 0.53 | 0.032 | 0.54 | 0.0E+00 | 0.0E+00 |
| Cmah | 6 | 0.546 | 0.084 | 0.54 | 0.0E+00 | 0.0E+00 |
| Dntt | 6 | 0.392 | 0.005 | 0.54 | 0.0E+00 | 0.0E+00 |
| Man1a2 | 6 | 0.548 | 0.099 | 0.54 | 0.0E+00 | 0.0E+00 |
| Adssl1 | 6 | 0.468 | 0.036 | 0.54 | 0.0E+00 | 0.0E+00 |
| Flt3 | 6 | 0.51 | 0.043 | 0.54 | 0.0E+00 | 0.0E+00 |
| Tgfbr1 | 6 | 0.52 | 0.086 | 0.54 | 0.0E+00 | 0.0E+00 |
| Tex2 | 6 | 0.522 | 0.043 | 0.53 | 0.0E+00 | 0.0E+00 |
| Atp13a2 | 6 | 0.507 | 0.046 | 0.53 | 0.0E+00 | 0.0E+00 |
| Dirc2 | 6 | 0.493 | 0.029 | 0.53 | 0.0E+00 | 0.0E+00 |
| Gas7 | 6 | 0.499 | 0.032 | 0.53 | 0.0E+00 | 0.0E+00 |
| Net1 | 6 | 0.485 | 0.044 | 0.53 | 0.0E+00 | 0.0E+00 |
| Npc1 | 6 | 0.484 | 0.029 | 0.53 | 0.0E+00 | 0.0E+00 |
| Il10rb | 6 | 0.59 | 0.14 | 0.52 | 0.0E+00 | 0.0E+00 |
| Clec9a | 6 | 0.445 | 0.01 | 0.52 | 0.0E+00 | 0.0E+00 |
| Lgals3 | 6 | 0.756 | 0.26 | 0.52 | 0.0E+00 | 0.0E+00 |
| Grina | 6 | 0.578 | 0.145 | 0.51 | 0.0E+00 | 0.0E+00 |
| Gna15 | 6 | 0.474 | 0.041 | 0.51 | 0.0E+00 | 0.0E+00 |
| Cdh5 | 6 | 0.413 | 0.009 | 0.50 | 0.0E+00 | 0.0E+00 |
| Ptprs | 6 | 0.458 | 0.021 | 0.50 | 0.0E+00 | 0.0E+00 |
| Mzb1 | 6 | 0.81 | 0.219 | 0.50 | 0.0E+00 | 0.0E+00 |
| BC147527 | 6 | 0.456 | 0.026 | 0.49 | 0.0E+00 | 0.0E+00 |
| Polr3gl | 6 | 0.515 | 0.105 | 0.49 | 0.0E+00 | 0.0E+00 |
| Ddr1 | 6 | 0.44 | 0.017 | 0.49 | 0.0E+00 | 0.0E+00 |
| Rassf4 | 6 | 0.458 | 0.035 | 0.48 | 0.0E+00 | 0.0E+00 |
| Fbxl13 | 6 | 0.435 | 0.004 | 0.48 | 0.0E+00 | 0.0E+00 |
| Rgs18 | 6 | 0.443 | 0.034 | 0.47 | 0.0E+00 | 0.0E+00 |
| Ccdc162 | 6 | 0.424 | 0.006 | 0.47 | 0.0E+00 | 0.0E+00 |
| Ccr5 | 6 | 0.442 | 0.049 | 0.46 | 0.0E+00 | 0.0E+00 |
| Nceh1 | 6 | 0.441 | 0.037 | 0.46 | 0.0E+00 | 0.0E+00 |
| 5430427O19Rik | 6 | 0.417 | 0.042 | 0.46 | 0.0E+00 | 0.0E+00 |
| Cd33 | 6 | 0.301 | 0.01 | 0.46 | 0.0E+00 | 0.0E+00 |
| Cacna1e | 6 | 0.409 | 0.013 | 0.45 | 0.0E+00 | 0.0E+00 |
| B3gnt8 | 6 | 0.424 | 0.033 | 0.45 | 0.0E+00 | 0.0E+00 |
| Stambpl1 | 6 | 0.433 | 0.054 | 0.45 | 0.0E+00 | 0.0E+00 |
| Mycl | 6 | 0.39 | 0.013 | 0.45 | 0.0E+00 | 0.0E+00 |
| Tlr7 | 6 | 0.405 | 0.009 | 0.45 | 0.0E+00 | 0.0E+00 |
| Slc29a3 | 6 | 0.424 | 0.024 | 0.45 | 0.0E+00 | 0.0E+00 |
| Rilpl1 | 6 | 0.418 | 0.013 | 0.44 | 0.0E+00 | 0.0E+00 |
| Lrrc16a | 6 | 0.415 | 0.014 | 0.44 | 0.0E+00 | 0.0E+00 |
| Eepd1 | 6 | 0.414 | 0.025 | 0.43 | 0.0E+00 | 0.0E+00 |
| Klhl42 | 6 | 0.417 | 0.053 | 0.42 | 0.0E+00 | 0.0E+00 |
| Tifab | 6 | 0.393 | 0.019 | 0.41 | 0.0E+00 | 0.0E+00 |
| Cd300lf | 6 | 0.414 | 0.031 | 0.40 | 0.0E+00 | 0.0E+00 |
| Pdzd4 | 6 | 0.368 | 0.004 | 0.39 | 0.0E+00 | 0.0E+00 |
| Tnni2 | 6 | 0.416 | 0.038 | 0.39 | 0.0E+00 | 0.0E+00 |
| Arhgap27os2 | 6 | 0.354 | 0.011 | 0.39 | 0.0E+00 | 0.0E+00 |
| Tmem163 | 6 | 0.368 | 0.012 | 0.38 | 0.0E+00 | 0.0E+00 |
| Tom1 | 6 | 0.391 | 0.036 | 0.38 | 0.0E+00 | 0.0E+00 |
| Pirb | 6 | 0.374 | 0.023 | 0.37 | 0.0E+00 | 0.0E+00 |
| Zeb2os | 6 | 0.351 | 0.014 | 0.37 | 0.0E+00 | 0.0E+00 |
| Atp1a3 | 6 | 0.339 | 0.017 | 0.37 | 0.0E+00 | 0.0E+00 |
| Rbm47 | 6 | 0.364 | 0.024 | 0.37 | 0.0E+00 | 0.0E+00 |
| Tcf7l2 | 6 | 0.287 | 0.012 | 0.32 | 0.0E+00 | 0.0E+00 |
| Plxdc1 | 6 | 0.295 | 0.007 | 0.31 | 0.0E+00 | 0.0E+00 |
| Ldlrad3 | 6 | 0.291 | 0.009 | 0.31 | 0.0E+00 | 0.0E+00 |
| Slco4a1 | 6 | 0.279 | 0.008 | 0.30 | 0.0E+00 | 0.0E+00 |
| Pnck | 6 | 0.268 | 0.005 | 0.29 | 0.0E+00 | 0.0E+00 |
| P2ry13 | 6 | 0.246 | 0.004 | 0.28 | 0.0E+00 | 0.0E+00 |
| P2ry6 | 6 | 0.277 | 0.01 | 0.28 | 0.0E+00 | 0.0E+00 |
| Cpne2 | 6 | 0.273 | 0.012 | 0.28 | 0.0E+00 | 0.0E+00 |
| Epha2 | 6 | 0.252 | 0.004 | 0.28 | 0.0E+00 | 0.0E+00 |
| B3galnt1 | 6 | 0.258 | 0.005 | 0.27 | 0.0E+00 | 0.0E+00 |
| Nbl1 | 6 | 0.256 | 0.01 | 0.27 | 0.0E+00 | 0.0E+00 |
| Rap1a | 6 | 0.94 | 0.466 | 0.90 | 1.3E−313 | 0.0E+00 |
| 9-Sep | 6 | 0.721 | 0.272 | 0.60 | 5.6E−313 | 0.0E+00 |
| Dclre1c | 6 | 0.31 | 0.024 | 0.33 | 2.7E−312 | 5.2E−308 |
| Ptpn6 | 6 | 0.892 | 0.426 | 0.89 | 2.0E−311 | 3.9E−307 |
| Cd8a | 6 | 0.436 | 0.084 | 0.78 | 2.9E−310 | 5.6E−306 |
| Snx9 | 6 | 0.417 | 0.05 | 0.40 | 0.0E+00 | 8.7E−305 |
| Ly6a | 6 | 0.275 | 0.019 | 0.83 | 0.0E+00 | 1.1E−304 |
| N4bp3 | 6 | 0.377 | 0.041 | 0.38 | 2.1E−307 | 4.1E−303 |
| Cxxc5 | 6 | 0.58 | 0.135 | 0.54 | 3.6E−306 | 6.8E−302 |
| Il7r | 6 | 0.757 | 0.371 | 0.30 | 1.6E−305 | 3.1E−301 |
| Asah1 | 6 | 0.433 | 0.053 | 0.42 | 1.5E−302 | 2.9E−298 |
| Ftl1 | 6 | 0.999 | 0.963 | 0.91 | 1.2E−301 | 2.4E−297 |
| Mgat1 | 6 | 0.589 | 0.125 | 0.54 | 1.4E−301 | 2.6E−297 |
| Cd14 | 6 | 0.262 | 0.017 | 0.25 | 1.7E−301 | 3.3E−297 |
| Itga4 | 6 | 0.716 | 0.214 | 0.69 | 1.8E−301 | 3.5E−297 |
| Bloc1s2 | 6 | 0.739 | 0.189 | 0.71 | 1.8E−299 | 3.5E−295 |
| Ifi44 | 6 | 0.248 | 0.011 | 0.35 | 1.6E−297 | 3.0E−293 |
| Rab7b | 6 | 0.24 | 0.011 | 0.28 | 2.9E−297 | 5.6E−293 |
| Zfp691 | 6 | 0.281 | 0.021 | 0.32 | 1.9E−296 | 3.7E−292 |
| Cib1 | 6 | 0.742 | 0.233 | 0.69 | 3.3E−295 | 6.3E−291 |
| Zyx | 6 | 0.623 | 0.157 | 0.61 | 3.3E−291 | 6.3E−287 |
| Ubc | 6 | 0.763 | 0.341 | 0.75 | 2.1E−290 | 4.0E−286 |
| Rabgap1l | 6 | 0.743 | 0.288 | 0.60 | 1.3E−289 | 2.6E−285 |
| Trf | 6 | 0.513 | 0.106 | 0.45 | 2.6E−289 | 4.9E−285 |
| Gpr137b-ps | 6 | 0.323 | 0.028 | 0.34 | 2.7E−289 | 5.1E−285 |
| Eps15 | 6 | 0.53 | 0.119 | 0.47 | 2.5E−288 | 4.8E−284 |
| Tgfb1 | 6 | 0.876 | 0.434 | 0.83 | 3.4E−288 | 6.6E−284 |
| Gns | 6 | 0.455 | 0.073 | 0.43 | 7.1E−288 | 1.4E−283 |
| Mapkapk2 | 6 | 0.574 | 0.138 | 0.55 | 5.4E−286 | 1.0E−281 |
| Adpgk | 6 | 0.409 | 0.066 | 0.37 | 9.8E−286 | 1.9E−281 |
| Tifa | 6 | 0.409 | 0.056 | 0.44 | 7.0E−285 | 1.3E−280 |
| Traf4 | 6 | 0.563 | 0.129 | 0.57 | 1.9E−284 | 3.7E−280 |
| Gne | 6 | 0.344 | 0.037 | 0.33 | 3.9E−284 | 7.6E−280 |
| Cd44 | 6 | 0.609 | 0.148 | 0.58 | 9.1E−284 | 1.7E−279 |
| Tbxa2r | 6 | 0.312 | 0.044 | 0.32 | 2.9E−283 | 5.5E−279 |
| Slc41a2 | 6 | 0.327 | 0.026 | 0.33 | 1.1E−280 | 2.0E−276 |
| Fdps | 6 | 0.637 | 0.156 | 0.80 | 1.1E−278 | 2.1E−274 |
| Nptn | 6 | 0.521 | 0.113 | 0.49 | 2.1E−276 | 4.0E−272 |
| 5730508B09Rik | 6 | 0.29 | 0.026 | 0.31 | 3.7E−276 | 7.2E−272 |
| Ifi44l | 6 | 0.229 | 0.01 | 0.27 | 1.8E−275 | 3.6E−271 |
| Lmo2 | 6 | 0.563 | 0.105 | 0.50 | 1.2E−274 | 2.4E−270 |
| Xist | 6 | 0.997 | 0.873 | 0.89 | 2.1E−274 | 4.0E−270 |
| Arhgap17 | 6 | 0.597 | 0.156 | 0.54 | 3.9E−273 | 7.5E−269 |
| Izumo1r | 7 | 0.861 | 0.073 | 1.51 | 0.0E+00 | 0.0E+00 |
| Trbc2 | 7 | 0.977 | 0.548 | 1.12 | 0.0E+00 | 0.0E+00 |
| Cd3g | 7 | 0.968 | 0.343 | 1.03 | 0.0E+00 | 0.0E+00 |
| Trac | 7 | 0.883 | 0.274 | 1.02 | 0.0E+00 | 0.0E+00 |
| Cd3e | 7 | 0.94 | 0.298 | 1.02 | 0.0E+00 | 0.0E+00 |
| Cd3d | 7 | 0.94 | 0.327 | 0.98 | 0.0E+00 | 0.0E+00 |
| Rgs10 | 7 | 0.857 | 0.472 | 0.87 | 0.0E+00 | 0.0E+00 |
| Tnfrsf4 | 7 | 0.47 | 0.038 | 0.80 | 0.0E+00 | 0.0E+00 |
| Ifi27l2a | 7 | 0.802 | 0.372 | 0.76 | 0.0E+00 | 0.0E+00 |
| Lat | 7 | 0.829 | 0.331 | 0.75 | 0.0E+00 | 0.0E+00 |
| Tnfsf8 | 7 | 0.365 | 0.064 | 0.69 | 0.0E+00 | 0.0E+00 |
| Lck | 7 | 0.882 | 0.477 | 0.69 | 0.0E+00 | 0.0E+00 |
| Ctla4 | 7 | 0.45 | 0.043 | 0.66 | 0.0E+00 | 0.0E+00 |
| Cd28 | 7 | 0.704 | 0.207 | 0.66 | 0.0E+00 | 0.0E+00 |
| Cd2 | 7 | 0.741 | 0.309 | 0.63 | 0.0E+00 | 0.0E+00 |
| Pdcd1 | 7 | 0.336 | 0.037 | 0.63 | 0.0E+00 | 0.0E+00 |
| Cd27 | 7 | 0.771 | 0.343 | 0.63 | 0.0E+00 | 0.0E+00 |
| Sh2d1a | 7 | 0.489 | 0.092 | 0.60 | 0.0E+00 | 0.0E+00 |
| Cdk2ap2 | 7 | 0.863 | 0.601 | 0.56 | 0.0E+00 | 0.0E+00 |
| Shisa5 | 7 | 0.991 | 0.872 | 0.54 | 0.0E+00 | 0.0E+00 |
| Fyb | 7 | 0.809 | 0.411 | 0.53 | 0.0E+00 | 0.0E+00 |
| Maf | 7 | 0.476 | 0.128 | 0.51 | 0.0E+00 | 0.0E+00 |
| Cd6 | 7 | 0.494 | 0.09 | 0.50 | 0.0E+00 | 0.0E+00 |
| Skap1 | 7 | 0.739 | 0.352 | 0.50 | 0.0E+00 | 0.0E+00 |
| Cd5 | 7 | 0.459 | 0.081 | 0.49 | 0.0E+00 | 0.0E+00 |
| Cd247 | 7 | 0.56 | 0.183 | 0.49 | 0.0E+00 | 0.0E+00 |
| Tbc1d4 | 7 | 0.413 | 0.055 | 0.42 | 0.0E+00 | 0.0E+00 |
| Hif1a | 7 | 0.444 | 0.185 | 0.41 | 0.0E+00 | 0.0E+00 |
| Cd4 | 7 | 0.485 | 0.145 | 0.38 | 2.3E−308 | 4.3E−304 |
| Tox | 7 | 0.435 | 0.155 | 0.38 | 3.5E−293 | 6.8E−289 |
| Foxp3 | 7 | 0.174 | 0.01 | 0.25 | 1.0E−286 | 2.0E−282 |
| Slamf6 | 7 | 0.506 | 0.215 | 0.42 | 1.8E−286 | 3.5E−282 |
| Ccl5 | 8 | 0.993 | 0.181 | 2.74 | 0.0E+00 | 0.0E+00 |
| Gzma | 8 | 0.745 | 0.069 | 2.38 | 0.0E+00 | 0.0E+00 |
| Nkg7 | 8 | 0.983 | 0.216 | 1.80 | 0.0E+00 | 0.0E+00 |
| AW112010 | 8 | 0.996 | 0.569 | 1.52 | 0.0E+00 | 0.0E+00 |
| Ncr1 | 8 | 0.951 | 0.125 | 1.42 | 0.0E+00 | 0.0E+00 |
| Ctla2a | 8 | 0.697 | 0.051 | 1.24 | 0.0E+00 | 0.0E+00 |
| Klrd1 | 8 | 0.908 | 0.135 | 1.20 | 0.0E+00 | 0.0E+00 |
| Ms4a4b | 8 | 0.932 | 0.382 | 0.93 | 0.0E+00 | 0.0E+00 |
| Klre1 | 8 | 0.703 | 0.016 | 0.93 | 0.0E+00 | 0.0E+00 |
| Ccl3 | 8 | 0.426 | 0.063 | 0.92 | 0.0E+00 | 0.0E+00 |
| Xcl1 | 8 | 0.532 | 0.077 | 0.90 | 0.0E+00 | 0.0E+00 |
| Klrb1c | 8 | 0.688 | 0.043 | 0.88 | 0.0E+00 | 0.0E+00 |
| Klrc1 | 8 | 0.548 | 0.045 | 0.87 | 0.0E+00 | 0.0E+00 |
| Il2rb | 8 | 0.802 | 0.192 | 0.86 | 0.0E+00 | 0.0E+00 |
| Klrk1 | 8 | 0.828 | 0.171 | 0.84 | 0.0E+00 | 0.0E+00 |
| Klra3 | 8 | 0.455 | 0.011 | 0.83 | 0.0E+00 | 0.0E+00 |
| Klri2 | 8 | 0.542 | 0.052 | 0.80 | 0.0E+00 | 0.0E+00 |
| Serpinb9 | 8 | 0.596 | 0.116 | 0.79 | 0.0E+00 | 0.0E+00 |
| Tyrobp | 8 | 0.989 | 0.275 | 0.74 | 0.0E+00 | 0.0E+00 |
| Vps37b | 8 | 0.804 | 0.398 | 0.73 | 0.0E+00 | 0.0E+00 |
| Ctsw | 8 | 0.789 | 0.226 | 0.73 | 0.0E+00 | 0.0E+00 |
| Gzmb | 8 | 0.336 | 0.029 | 0.71 | 0.0E+00 | 0.0E+00 |
| Eomes | 8 | 0.578 | 0.021 | 0.71 | 0.0E+00 | 0.0E+00 |
| Fcer1g | 8 | 0.921 | 0.344 | 0.69 | 0.0E+00 | 0.0E+00 |
| Ccl4 | 8 | 0.579 | 0.159 | 0.66 | 0.0E+00 | 0.0E+00 |
| Serpinb6b | 8 | 0.598 | 0.143 | 0.66 | 0.0E+00 | 0.0E+00 |
| Ctsd | 8 | 0.764 | 0.316 | 0.65 | 0.0E+00 | 0.0E+00 |
| Nabp1 | 8 | 0.694 | 0.261 | 0.63 | 0.0E+00 | 0.0E+00 |
| Gimap4 | 8 | 0.861 | 0.453 | 0.62 | 0.0E+00 | 0.0E+00 |
| Ugcg | 8 | 0.625 | 0.122 | 0.62 | 0.0E+00 | 0.0E+00 |
| Fasl | 8 | 0.552 | 0.061 | 0.59 | 0.0E+00 | 0.0E+00 |
| Klra7 | 8 | 0.284 | 0.006 | 0.58 | 0.0E+00 | 0.0E+00 |
| Dok2 | 8 | 0.648 | 0.192 | 0.57 | 0.0E+00 | 0.0E+00 |
| Jak1 | 8 | 0.781 | 0.445 | 0.56 | 0.0E+00 | 0.0E+00 |
| Klra9 | 8 | 0.364 | 0.005 | 0.56 | 0.0E+00 | 0.0E+00 |
| Irf8 | 8 | 0.899 | 0.385 | 0.55 | 0.0E+00 | 0.0E+00 |
| Klrc2 | 8 | 0.452 | 0.036 | 0.54 | 0.0E+00 | 0.0E+00 |
| Sh2d2a | 8 | 0.537 | 0.17 | 0.54 | 0.0E+00 | 0.0E+00 |
| Ifngr1 | 8 | 0.833 | 0.423 | 0.51 | 0.0E+00 | 0.0E+00 |
| Ctla2b | 8 | 0.418 | 0.019 | 0.49 | 0.0E+00 | 0.0E+00 |
| Klrb1f | 8 | 0.491 | 0.089 | 0.48 | 0.0E+00 | 0.0E+00 |
| Ccr2 | 8 | 0.475 | 0.092 | 0.46 | 0.0E+00 | 0.0E+00 |
| Anxa2 | 8 | 0.625 | 0.16 | 0.46 | 0.0E+00 | 0.0E+00 |
| Ptprc | 8 | 0.904 | 0.632 | 0.46 | 0.0E+00 | 0.0E+00 |
| Samd3 | 8 | 0.402 | 0.018 | 0.43 | 0.0E+00 | 0.0E+00 |
| Ccr5 | 8 | 0.413 | 0.056 | 0.43 | 0.0E+00 | 0.0E+00 |
| Pglyrp1 | 8 | 0.51 | 0.108 | 0.42 | 0.0E+00 | 0.0E+00 |
| Klrb1a | 8 | 0.338 | 0.026 | 0.40 | 0.0E+00 | 0.0E+00 |
| Tm6sf1 | 8 | 0.404 | 0.07 | 0.36 | 0.0E+00 | 0.0E+00 |
| Trdc | 8 | 0.37 | 0.032 | 0.35 | 0.0E+00 | 0.0E+00 |
| Ifitm10 | 8 | 0.339 | 0.016 | 0.35 | 0.0E+00 | 0.0E+00 |
| Metrnl | 8 | 0.325 | 0.037 | 0.35 | 0.0E+00 | 0.0E+00 |
| 1700025G04Rik | 8 | 0.346 | 0.048 | 0.32 | 0.0E+00 | 0.0E+00 |
| H2afz | 8 | 0.965 | 0.731 | 0.32 | 0.0E+00 | 0.0E+00 |
| Cma1 | 8 | 0.215 | 0.006 | 0.30 | 0.0E+00 | 0.0E+00 |
| Prf1 | 8 | 0.232 | 0.011 | 0.28 | 0.0E+00 | 0.0E+00 |
| Chsy1 | 8 | 0.33 | 0.056 | 0.30 | 7.6E−311 | 1.5E−306 |
| Selplg | 8 | 0.918 | 0.585 | 0.46 | 0.0E+00 | 4.6E−305 |
| Cd7 | 8 | 0.779 | 0.361 | 0.56 | 2.7E−297 | 5.3E−293 |
| Ahnak | 8 | 0.519 | 0.164 | 0.37 | 4.3E−292 | 8.2E−288 |
| Calm2 | 8 | 0.916 | 0.691 | 0.31 | 5.5E−284 | 1.1E−279 |
| Txk | 8 | 0.751 | 0.353 | 0.52 | 2.1E−278 | 4.0E−274 |
| S100a10 | 8 | 0.899 | 0.574 | 0.47 | 6.0E−273 | 1.2E−268 |
| Sh2d1a | 8 | 0.438 | 0.1 | 0.38 | 8.2E−272 | 1.6E−267 |
| Hmgb2 | 9 | 0.999 | 0.513 | 2.75 | 0.0E+00 | 0.0E+00 |
| 2810417H13Rik | 9 | 0.919 | 0.038 | 2.64 | 0.0E+00 | 0.0E+00 |
| Stmn1 | 9 | 0.977 | 0.103 | 2.49 | 0.0E+00 | 0.0E+00 |
| Tuba1b | 9 | 0.948 | 0.469 | 2.38 | 0.0E+00 | 0.0E+00 |
| Tubb5 | 9 | 0.971 | 0.589 | 2.28 | 0.0E+00 | 0.0E+00 |
| Ptma | 9 | 1 | 0.958 | 2.21 | 0.0E+00 | 0.0E+00 |
| H2afz | 9 | 0.998 | 0.73 | 2.19 | 0.0E+00 | 0.0E+00 |
| Ms4a1 | 9 | 0.995 | 0.178 | 2.07 | 0.0E+00 | 0.0E+00 |
| H2afx | 9 | 0.861 | 0.17 | 2.05 | 0.0E+00 | 0.0E+00 |
| Hmgn2 | 9 | 0.99 | 0.328 | 2.03 | 0.0E+00 | 0.0E+00 |
| Cd24a | 9 | 0.976 | 0.187 | 1.92 | 0.0E+00 | 0.0E+00 |
| Hmgn1 | 9 | 0.997 | 0.485 | 1.89 | 0.0E+00 | 0.0E+00 |
| Cd79b | 9 | 0.998 | 0.272 | 1.87 | 0.0E+00 | 0.0E+00 |
| Ran | 9 | 0.995 | 0.586 | 1.86 | 0.0E+00 | 0.0E+00 |
| Nap1l1 | 9 | 0.997 | 0.432 | 1.85 | 0.0E+00 | 0.0E+00 |
| Ighg1 | 9 | 0.891 | 0.111 | 1.84 | 0.0E+00 | 0.0E+00 |
| Hmces | 9 | 0.915 | 0.131 | 1.84 | 0.0E+00 | 0.0E+00 |
| Hmgb1 | 9 | 0.995 | 0.764 | 1.83 | 0.0E+00 | 0.0E+00 |
| Rgs13 | 9 | 0.948 | 0.065 | 1.83 | 0.0E+00 | 0.0E+00 |
| Gapdh | 9 | 0.998 | 0.746 | 1.83 | 0.0E+00 | 0.0E+00 |
| Cd79a | 9 | 0.997 | 0.232 | 1.81 | 0.0E+00 | 0.0E+00 |
| Pou2af1 | 9 | 0.963 | 0.124 | 1.81 | 0.0E+00 | 0.0E+00 |
| Anp32b | 9 | 0.994 | 0.537 | 1.81 | 0.0E+00 | 0.0E+00 |
| Mzb1 | 9 | 0.985 | 0.22 | 1.76 | 0.0E+00 | 0.0E+00 |
| Cks2 | 9 | 0.807 | 0.116 | 1.74 | 0.0E+00 | 0.0E+00 |
| Arpc5l | 9 | 0.991 | 0.393 | 1.74 | 0.0E+00 | 0.0E+00 |
| Vpreb3 | 9 | 0.894 | 0.128 | 1.69 | 0.0E+00 | 0.0E+00 |
| Ebf1 | 9 | 0.987 | 0.193 | 1.69 | 0.0E+00 | 0.0E+00 |
| Ube2s | 9 | 0.947 | 0.464 | 1.66 | 0.0E+00 | 0.0E+00 |
| H3f3a | 9 | 1 | 0.925 | 1.64 | 0.0E+00 | 0.0E+00 |
| Dut | 9 | 0.883 | 0.181 | 1.63 | 0.0E+00 | 0.0E+00 |
| Pold4 | 9 | 0.988 | 0.343 | 1.63 | 0.0E+00 | 0.0E+00 |
| Slc25a5 | 9 | 0.997 | 0.7 | 1.62 | 0.0E+00 | 0.0E+00 |
| H2afv | 9 | 0.952 | 0.406 | 1.61 | 0.0E+00 | 0.0E+00 |
| Txn1 | 9 | 0.987 | 0.501 | 1.60 | 0.0E+00 | 0.0E+00 |
| Rbm3 | 9 | 0.999 | 0.819 | 1.59 | 0.0E+00 | 0.0E+00 |
| Hnrnpa2b1 | 9 | 0.996 | 0.712 | 1.59 | 0.0E+00 | 0.0E+00 |
| Chchd10 | 9 | 0.916 | 0.214 | 1.58 | 0.0E+00 | 0.0E+00 |
| Crip1 | 9 | 0.997 | 0.578 | 1.58 | 0.0E+00 | 0.0E+00 |
| Erh | 9 | 0.985 | 0.542 | 1.56 | 0.0E+00 | 0.0E+00 |
| Cks1b | 9 | 0.843 | 0.066 | 1.55 | 0.0E+00 | 0.0E+00 |
| Glrx3 | 9 | 0.971 | 0.316 | 1.54 | 0.0E+00 | 0.0E+00 |
| Prdx1 | 9 | 0.994 | 0.646 | 1.53 | 0.0E+00 | 0.0E+00 |
| Basp1 | 9 | 0.922 | 0.129 | 1.53 | 0.0E+00 | 0.0E+00 |
| Ranbp1 | 9 | 0.927 | 0.449 | 1.53 | 0.0E+00 | 0.0E+00 |
| Klhl6 | 9 | 0.934 | 0.239 | 1.52 | 0.0E+00 | 0.0E+00 |
| Birc5 | 9 | 0.688 | 0.019 | 1.51 | 0.0E+00 | 0.0E+00 |
| Tubb4b | 9 | 0.79 | 0.249 | 1.50 | 0.0E+00 | 0.0E+00 |
| Gcsam | 9 | 0.874 | 0.055 | 1.50 | 0.0E+00 | 0.0E+00 |
| Hnrnpa3 | 9 | 0.985 | 0.603 | 1.49 | 0.0E+00 | 0.0E+00 |
| Cmpk1 | 9 | 0.929 | 0.319 | 1.47 | 0.0E+00 | 0.0E+00 |
| Snrpe | 9 | 0.993 | 0.614 | 1.47 | 0.0E+00 | 0.0E+00 |
| 2700094K13Rik | 9 | 0.918 | 0.267 | 1.47 | 0.0E+00 | 0.0E+00 |
| Rrm2 | 9 | 0.591 | 0.013 | 1.47 | 0.0E+00 | 0.0E+00 |
| Slbp | 9 | 0.89 | 0.299 | 1.47 | 0.0E+00 | 0.0E+00 |
| Eaf2 | 9 | 0.9 | 0.061 | 1.46 | 0.0E+00 | 0.0E+00 |
| Clic1 | 9 | 0.997 | 0.716 | 1.43 | 0.0E+00 | 0.0E+00 |
| Pkig | 9 | 0.934 | 0.231 | 1.43 | 0.0E+00 | 0.0E+00 |
| Uchl3 | 9 | 0.922 | 0.226 | 1.42 | 0.0E+00 | 0.0E+00 |
| Hnrnpk | 9 | 0.994 | 0.73 | 1.42 | 0.0E+00 | 0.0E+00 |
| Ube2c | 9 | 0.546 | 0.017 | 1.42 | 0.0E+00 | 0.0E+00 |
| Srsf3 | 9 | 0.991 | 0.627 | 1.41 | 0.0E+00 | 0.0E+00 |
| Sypl | 9 | 0.962 | 0.201 | 1.41 | 0.0E+00 | 0.0E+00 |
| Snrpd1 | 9 | 0.95 | 0.327 | 1.40 | 0.0E+00 | 0.0E+00 |
| Ppp1ca | 9 | 0.993 | 0.734 | 1.40 | 0.0E+00 | 0.0E+00 |
| Atp5b | 9 | 0.99 | 0.727 | 1.40 | 0.0E+00 | 0.0E+00 |
| Aicda | 9 | 0.88 | 0.053 | 1.39 | 0.0E+00 | 0.0E+00 |
| Top1 | 9 | 0.962 | 0.383 | 1.39 | 0.0E+00 | 0.0E+00 |
| Dek | 9 | 0.91 | 0.325 | 1.39 | 0.0E+00 | 0.0E+00 |
| Lrmp | 9 | 0.924 | 0.245 | 1.38 | 0.0E+00 | 0.0E+00 |
| Snrpg | 9 | 0.995 | 0.784 | 1.38 | 0.0E+00 | 0.0E+00 |
| Hsp90aa1 | 9 | 0.976 | 0.629 | 1.37 | 0.0E+00 | 0.0E+00 |
| Polr2g | 9 | 0.938 | 0.316 | 1.37 | 0.0E+00 | 0.0E+00 |
| Apitd1 | 9 | 0.894 | 0.08 | 1.36 | 0.0E+00 | 0.0E+00 |
| Tnfrsf13c | 9 | 0.906 | 0.147 | 1.35 | 0.0E+00 | 0.0E+00 |
| Anp32e | 9 | 0.9 | 0.249 | 1.35 | 0.0E+00 | 0.0E+00 |
| Lmnb1 | 9 | 0.847 | 0.111 | 1.33 | 0.0E+00 | 0.0E+00 |
| Gtf2a2 | 9 | 0.942 | 0.298 | 1.33 | 0.0E+00 | 0.0E+00 |
| Top2a | 9 | 0.667 | 0.023 | 1.32 | 0.0E+00 | 0.0E+00 |
| H1fx | 9 | 0.675 | 0.019 | 1.32 | 0.0E+00 | 0.0E+00 |
| Cdc20 | 9 | 0.522 | 0.021 | 1.32 | 0.0E+00 | 0.0E+00 |
| Psip1 | 9 | 0.918 | 0.232 | 1.32 | 0.0E+00 | 0.0E+00 |
| Tcp1 | 9 | 0.955 | 0.36 | 1.32 | 0.0E+00 | 0.0E+00 |
| Srsf2 | 9 | 0.962 | 0.554 | 1.32 | 0.0E+00 | 0.0E+00 |
| Atp5a1 | 9 | 0.986 | 0.663 | 1.31 | 0.0E+00 | 0.0E+00 |
| Mcm5 | 9 | 0.77 | 0.065 | 1.31 | 0.0E+00 | 0.0E+00 |
| 2700029M09Rik | 9 | 0.883 | 0.183 | 1.31 | 0.0E+00 | 0.0E+00 |
| Rhoh | 9 | 0.969 | 0.478 | 1.30 | 0.0E+00 | 0.0E+00 |
| Scimp | 9 | 0.933 | 0.137 | 1.30 | 0.0E+00 | 0.0E+00 |
| Bfsp2 | 9 | 0.775 | 0.057 | 1.30 | 0.0E+00 | 0.0E+00 |
| Smagp | 9 | 0.866 | 0.055 | 1.28 | 0.0E+00 | 0.0E+00 |
| Cdca8 | 9 | 0.672 | 0.02 | 1.28 | 0.0E+00 | 0.0E+00 |
| Hnrnpf | 9 | 0.998 | 0.826 | 1.28 | 0.0E+00 | 0.0E+00 |
| Dynll1 | 9 | 0.988 | 0.68 | 1.27 | 0.0E+00 | 0.0E+00 |
| Ccnb2 | 9 | 0.623 | 0.013 | 1.27 | 0.0E+00 | 0.0E+00 |
| Grb2 | 9 | 0.941 | 0.347 | 1.26 | 0.0E+00 | 0.0E+00 |
| Pcna | 9 | 0.759 | 0.171 | 1.26 | 0.0E+00 | 0.0E+00 |
| Dbi | 9 | 0.947 | 0.301 | 1.26 | 0.0E+00 | 0.0E+00 |
| Rfc2 | 9 | 0.876 | 0.241 | 1.25 | 0.0E+00 | 0.0E+00 |
| Snrpf | 9 | 0.962 | 0.514 | 1.25 | 0.0E+00 | 0.0E+00 |
| Calm3 | 9 | 0.938 | 0.429 | 1.25 | 0.0E+00 | 0.0E+00 |
| Asf1b | 9 | 0.75 | 0.034 | 1.24 | 0.0E+00 | 0.0E+00 |
| Dnajc9 | 9 | 0.868 | 0.22 | 1.24 | 0.0E+00 | 0.0E+00 |
| Lsm5 | 9 | 0.938 | 0.398 | 1.22 | 0.0E+00 | 0.0E+00 |
| Snrpb | 9 | 0.977 | 0.641 | 1.21 | 0.0E+00 | 0.0E+00 |
| Mcm6 | 9 | 0.72 | 0.057 | 1.20 | 0.0E+00 | 0.0E+00 |
| Srsf7 | 9 | 0.92 | 0.365 | 1.20 | 0.0E+00 | 0.0E+00 |
| Pttg1 | 9 | 0.642 | 0.043 | 1.19 | 0.0E+00 | 0.0E+00 |
| Tipin | 9 | 0.781 | 0.103 | 1.19 | 0.0E+00 | 0.0E+00 |
| Nudt21 | 9 | 0.94 | 0.338 | 1.19 | 0.0E+00 | 0.0E+00 |
| Lsm6 | 9 | 0.926 | 0.337 | 1.19 | 0.0E+00 | 0.0E+00 |
| Hnrnpu | 9 | 0.916 | 0.399 | 1.18 | 0.0E+00 | 0.0E+00 |
| Ube2d2a | 9 | 0.981 | 0.56 | 1.18 | 0.0E+00 | 0.0E+00 |
| Lsm4 | 9 | 0.927 | 0.417 | 1.18 | 0.0E+00 | 0.0E+00 |
| Zfp706 | 9 | 0.956 | 0.492 | 1.16 | 0.0E+00 | 0.0E+00 |
| Neil1 | 9 | 0.792 | 0.078 | 1.15 | 0.0E+00 | 0.0E+00 |
| Alyref | 9 | 0.807 | 0.21 | 1.15 | 0.0E+00 | 0.0E+00 |
| Nusap1 | 9 | 0.568 | 0.009 | 1.15 | 0.0E+00 | 0.0E+00 |
| Myl4 | 9 | 0.714 | 0.052 | 1.15 | 0.0E+00 | 0.0E+00 |
| Smc4 | 9 | 0.795 | 0.286 | 1.15 | 0.0E+00 | 0.0E+00 |
| Ube2n | 9 | 0.94 | 0.393 | 1.13 | 0.0E+00 | 0.0E+00 |
| Erp44 | 9 | 0.891 | 0.257 | 1.13 | 0.0E+00 | 0.0E+00 |
| Ppp1cc | 9 | 0.943 | 0.485 | 1.13 | 0.0E+00 | 0.0E+00 |
| Lig1 | 9 | 0.693 | 0.056 | 1.12 | 0.0E+00 | 0.0E+00 |
| U2af1 | 9 | 0.916 | 0.393 | 1.12 | 0.0E+00 | 0.0E+00 |
| Tmpo | 9 | 0.832 | 0.185 | 1.12 | 0.0E+00 | 0.0E+00 |
| Ddx39 | 9 | 0.853 | 0.215 | 1.12 | 0.0E+00 | 0.0E+00 |
| Vdac3 | 9 | 0.896 | 0.271 | 1.11 | 0.0E+00 | 0.0E+00 |
| Mcm7 | 9 | 0.739 | 0.075 | 1.11 | 0.0E+00 | 0.0E+00 |
| Ewsr1 | 9 | 0.923 | 0.386 | 1.11 | 0.0E+00 | 0.0E+00 |
| Rbbp7 | 9 | 0.828 | 0.259 | 1.11 | 0.0E+00 | 0.0E+00 |
| Sfpq | 9 | 0.916 | 0.393 | 1.08 | 0.0E+00 | 0.0E+00 |
| Bub3 | 9 | 0.841 | 0.301 | 1.08 | 0.0E+00 | 0.0E+00 |
| Nasp | 9 | 0.764 | 0.129 | 1.08 | 0.0E+00 | 0.0E+00 |
| Csk | 9 | 0.939 | 0.449 | 1.07 | 0.0E+00 | 0.0E+00 |
| Mif4gd | 9 | 0.887 | 0.301 | 1.07 | 0.0E+00 | 0.0E+00 |
| Spc24 | 9 | 0.642 | 0.025 | 1.07 | 0.0E+00 | 0.0E+00 |
| Mtf2 | 9 | 0.823 | 0.142 | 1.07 | 0.0E+00 | 0.0E+00 |
| Tcf3 | 9 | 0.844 | 0.182 | 1.07 | 0.0E+00 | 0.0E+00 |
| Mcm3 | 9 | 0.7 | 0.051 | 1.06 | 0.0E+00 | 0.0E+00 |
| Iglc3 | 9 | 0.948 | 0.249 | 1.05 | 0.0E+00 | 0.0E+00 |
| Gmnn | 9 | 0.713 | 0.062 | 1.04 | 0.0E+00 | 0.0E+00 |
| Nucks1 | 9 | 0.808 | 0.273 | 1.04 | 0.0E+00 | 0.0E+00 |
| Ccna2 | 9 | 0.533 | 0.007 | 1.03 | 0.0E+00 | 0.0E+00 |
| Dck | 9 | 0.774 | 0.069 | 1.03 | 0.0E+00 | 0.0E+00 |
| Phf5a | 9 | 0.865 | 0.262 | 1.03 | 0.0E+00 | 0.0E+00 |
| Dazap1 | 9 | 0.878 | 0.307 | 1.02 | 0.0E+00 | 0.0E+00 |
| Hprt | 9 | 0.862 | 0.329 | 1.02 | 0.0E+00 | 0.0E+00 |
| Mki67 | 9 | 0.631 | 0.017 | 1.01 | 0.0E+00 | 0.0E+00 |
| Bzw2 | 9 | 0.854 | 0.231 | 1.01 | 0.0E+00 | 0.0E+00 |
| Ezh2 | 9 | 0.732 | 0.083 | 1.01 | 0.0E+00 | 0.0E+00 |
| Akr1b3 | 9 | 0.896 | 0.396 | 0.99 | 0.0E+00 | 0.0E+00 |
| Rbmxl1 | 9 | 0.802 | 0.187 | 0.99 | 0.0E+00 | 0.0E+00 |
| Lsm3 | 9 | 0.838 | 0.251 | 0.99 | 0.0E+00 | 0.0E+00 |
| Cdca3 | 9 | 0.597 | 0.013 | 0.98 | 0.0E+00 | 0.0E+00 |
| Ssrp1 | 9 | 0.818 | 0.282 | 0.98 | 0.0E+00 | 0.0E+00 |
| Lsm2 | 9 | 0.789 | 0.195 | 0.98 | 0.0E+00 | 0.0E+00 |
| Tk1 | 9 | 0.631 | 0.02 | 0.98 | 0.0E+00 | 0.0E+00 |
| Tmem243 | 9 | 0.86 | 0.28 | 0.97 | 0.0E+00 | 0.0E+00 |
| Cdk1 | 9 | 0.53 | 0.01 | 0.97 | 0.0E+00 | 0.0E+00 |
| Tcea1 | 9 | 0.848 | 0.264 | 0.97 | 0.0E+00 | 0.0E+00 |
| Csrp2 | 9 | 0.742 | 0.043 | 0.96 | 0.0E+00 | 0.0E+00 |
| Tra2b | 9 | 0.956 | 0.568 | 0.96 | 0.0E+00 | 0.0E+00 |
| Elof1 | 9 | 0.828 | 0.2 | 0.96 | 0.0E+00 | 0.0E+00 |
| Apobec1 | 9 | 0.788 | 0.092 | 0.96 | 0.0E+00 | 0.0E+00 |
| Mcm2 | 9 | 0.674 | 0.074 | 0.94 | 0.0E+00 | 0.0E+00 |
| Cd19 | 9 | 0.801 | 0.106 | 0.94 | 0.0E+00 | 0.0E+00 |
| Rfc1 | 9 | 0.765 | 0.125 | 0.94 | 0.0E+00 | 0.0E+00 |
| Rbbp4 | 9 | 0.815 | 0.333 | 0.94 | 0.0E+00 | 0.0E+00 |
| Sh2b2 | 9 | 0.724 | 0.061 | 0.91 | 0.0E+00 | 0.0E+00 |
| H2-Ob | 9 | 0.804 | 0.141 | 0.89 | 0.0E+00 | 0.0E+00 |
| Rfc3 | 9 | 0.657 | 0.052 | 0.89 | 0.0E+00 | 0.0E+00 |
| Rrm1 | 9 | 0.616 | 0.041 | 0.89 | 0.0E+00 | 0.0E+00 |
| S1pr2 | 9 | 0.705 | 0.047 | 0.87 | 0.0E+00 | 0.0E+00 |
| Dhfr | 9 | 0.601 | 0.024 | 0.87 | 0.0E+00 | 0.0E+00 |
| Cenpm | 9 | 0.615 | 0.017 | 0.85 | 0.0E+00 | 0.0E+00 |
| Lipc | 9 | 0.66 | 0.032 | 0.83 | 0.0E+00 | 0.0E+00 |
| Lbr | 9 | 0.751 | 0.23 | 0.81 | 0.0E+00 | 0.0E+00 |
| Igkc | 9 | 1 | 0.693 | 0.77 | 0.0E+00 | 0.0E+00 |
| Jchain | 9 | 0.99 | 0.207 | 0.76 | 0.0E+00 | 0.0E+00 |
| Tpx2 | 9 | 0.456 | 0.008 | 0.75 | 0.0E+00 | 0.0E+00 |
| Mad2l1 | 9 | 0.605 | 0.035 | 0.74 | 0.0E+00 | 0.0E+00 |
| Tacc3 | 9 | 0.565 | 0.018 | 0.73 | 0.0E+00 | 0.0E+00 |
| Nuggc | 9 | 0.586 | 0.026 | 0.72 | 0.0E+00 | 0.0E+00 |
| Cenpe | 9 | 0.459 | 0.008 | 0.71 | 0.0E+00 | 0.0E+00 |
| Cep55 | 9 | 0.488 | 0.006 | 0.66 | 0.0E+00 | 0.0E+00 |
| Fcmr | 9 | 0.731 | 0.145 | 0.63 | 0.0E+00 | 0.0E+00 |
| Ctsh | 9 | 0.886 | 0.287 | 0.58 | 0.0E+00 | 0.0E+00 |
| Esco2 | 9 | 0.388 | 0.003 | 0.58 | 0.0E+00 | 0.0E+00 |
| Ncapg | 9 | 0.47 | 0.01 | 0.58 | 0.0E+00 | 0.0E+00 |
| Gpx1 | 9 | 0.887 | 0.572 | 0.46 | 0.0E+00 | 0.0E+00 |
| Ftl1 | 9 | 0.999 | 0.964 | 0.44 | 0.0E+00 | 0.0E+00 |
| Cyba | 9 | 0.929 | 0.769 | 0.31 | 0.0E+00 | 0.0E+00 |
| Lsp1 | 9 | 0.889 | 0.674 | 0.31 | 0.0E+00 | 0.0E+00 |
| Dok3 | 9 | 0.723 | 0.102 | 0.87 | 1.0E−313 | 0.0E+00 |
| Ssr4 | 9 | 0.754 | 0.529 | 0.27 | 1.2E−310 | 2.4E−306 |
| Nono | 9 | 0.919 | 0.408 | 1.01 | 0.0E+00 | 8.6E−305 |
| Cdca2 | 9 | 0.411 | 0.008 | 0.60 | 0.0E+00 | 4.1E−304 |
| Gdi2 | 9 | 0.987 | 0.532 | 1.35 | 5.2E−308 | 1.0E−303 |
| Fen1 | 9 | 0.662 | 0.069 | 0.88 | 8.3E−308 | 1.6E−303 |
| Xrcc1 | 9 | 0.709 | 0.093 | 0.82 | 6.1E−307 | 1.2E−302 |
| Nedd8 | 9 | 0.981 | 0.604 | 1.18 | 1.4E−306 | 2.8E−302 |
| Iglc2 | 9 | 0.847 | 0.209 | 0.97 | 2.8E−306 | 5.4E−302 |
| Mybl2 | 9 | 0.417 | 0.005 | 0.59 | 6.4E−305 | 1.2E−300 |
| Sumo2 | 9 | 0.99 | 0.781 | 1.30 | 8.3E−305 | 1.6E−300 |
| Trp53i11 | 9 | 0.794 | 0.148 | 0.98 | 2.6E−304 | 5.0E−300 |
| Mybl1 | 9 | 0.438 | 0.016 | 0.53 | 3.0E−304 | 5.7E−300 |
| Cdkn3 | 9 | 0.425 | 0.008 | 0.74 | 3.1E−301 | 6.0E−297 |
| Ybx1 | 9 | 0.998 | 0.788 | 1.62 | 1.7E−299 | 3.3E−295 |
| Cd37 | 9 | 0.975 | 0.568 | 1.10 | 6.7E−299 | 1.3E−294 |
| Cenpa | 9 | 0.617 | 0.112 | 1.06 | 6.8E−297 | 1.3E−292 |
| Cd22 | 9 | 0.642 | 0.079 | 0.72 | 1.4E−296 | 2.8E−292 |
| Arhgap8 | 9 | 0.538 | 0.025 | 0.62 | 2.3E−295 | 4.4E−291 |
| Aurkb | 9 | 0.473 | 0.012 | 0.72 | 3.6E−295 | 6.9E−291 |
| Atp5j | 9 | 0.979 | 0.648 | 1.20 | 9.4E−294 | 1.8E−289 |
| Mtmr14 | 9 | 0.771 | 0.099 | 1.00 | 1.2E−292 | 2.3E−288 |
| Prim1 | 9 | 0.548 | 0.034 | 0.68 | 1.1E−289 | 2.2E−285 |
| Ppp4r2 | 9 | 0.765 | 0.154 | 0.85 | 1.2E−289 | 2.2E−285 |
| Rfc5 | 9 | 0.641 | 0.065 | 0.82 | 7.4E−289 | 1.4E−284 |
| Pmf1 | 9 | 0.753 | 0.118 | 0.85 | 8.4E−289 | 1.6E−284 |
| Arpp19 | 9 | 0.952 | 0.559 | 1.06 | 7.0E−286 | 1.4E−281 |
| Hnrnpa1 | 9 | 0.985 | 0.616 | 1.36 | 2.1E−285 | 4.0E−281 |
| H2afj | 9 | 0.766 | 0.568 | 0.30 | 2.7E−285 | 5.2E−281 |
| Atp5k | 9 | 0.962 | 0.518 | 1.16 | 8.7E−285 | 1.7E−280 |
| Ublcp1 | 9 | 0.695 | 0.116 | 0.83 | 2.8E−284 | 5.5E−280 |
| Raly | 9 | 0.94 | 0.526 | 1.03 | 7.7E−283 | 1.5E−278 |
| Nuf2 | 9 | 0.38 | 0.004 | 0.52 | 1.3E−281 | 2.5E−277 |
| Hist1h2ap | 9 | 0.555 | 0.062 | 2.33 | 9.2E−281 | 1.8E−276 |
| Cdc25b | 9 | 0.565 | 0.056 | 0.71 | 3.8E−280 | 7.4E−276 |
| H2-DMb2 | 9 | 0.98 | 0.275 | 1.39 | 2.0E−279 | 3.8E−275 |
| Atp6v0e | 9 | 0.84 | 0.619 | 0.38 | 4.1E−279 | 7.9E−275 |
| Id3 | 9 | 0.722 | 0.112 | 1.01 | 5.2E−279 | 1.0E−274 |
| Snrpa1 | 9 | 0.805 | 0.2 | 1.00 | 3.5E−278 | 6.8E−274 |
| Uhrf1 | 9 | 0.502 | 0.016 | 0.72 | 6.8E−278 | 1.3E−273 |
| Ccnb1 | 9 | 0.403 | 0.008 | 0.82 | 2.2E−277 | 4.3E−273 |
| Cdk4 | 9 | 0.835 | 0.3 | 1.12 | 2.2E−276 | 4.3E−272 |
| Racgap1 | 9 | 0.546 | 0.041 | 0.81 | 1.1E−275 | 2.1E−271 |
| Set | 9 | 0.936 | 0.524 | 1.22 | 8.7E−275 | 1.7E−270 |
| Irf8 | 9 | 0.963 | 0.383 | 0.56 | 1.6E−274 | 3.1E−270 |
| Smc2 | 9 | 0.594 | 0.061 | 0.84 | 3.3E−274 | 6.3E−270 |
| Mbd4 | 9 | 0.529 | 0.033 | 0.58 | 2.0E−273 | 3.8E−269 |
| Fignl1 | 9 | 0.394 | 0.007 | 0.44 | 3.5E−273 | 6.7E−269 |
| Pbk | 9 | 0.376 | 0.004 | 0.56 | 6.0E−273 | 1.1E−268 |
| Polr1d | 9 | 0.961 | 0.564 | 1.09 | 6.9E−273 | 1.3E−268 |
| Atp5g2 | 9 | 0.991 | 0.741 | 1.31 | 8.6E−273 | 1.7E−268 |
| Ucp2 | 9 | 0.996 | 0.747 | 1.40 | 1.0E−272 | 2.0E−268 |
| Ckap2l | 9 | 0.37 | 0.005 | 0.51 | 2.3E−272 | 4.4E−268 |
| Hells | 9 | 0.582 | 0.048 | 0.79 | 7.1E−272 | 1.4E−267 |
| Ms4a1 | 10 | 0.991 | 0.178 | 1.67 | 0.0E+00 | 0.0E+00 |
| Cd79b | 10 | 0.995 | 0.273 | 1.67 | 0.0E+00 | 0.0E+00 |
| Cd79a | 10 | 0.998 | 0.232 | 1.51 | 0.0E+00 | 0.0E+00 |
| Ebf1 | 10 | 0.992 | 0.193 | 1.40 | 0.0E+00 | 0.0E+00 |
| Ighg1 | 10 | 0.844 | 0.114 | 1.39 | 0.0E+00 | 0.0E+00 |
| Rgs13 | 10 | 0.907 | 0.068 | 1.31 | 0.0E+00 | 0.0E+00 |
| Cd74 | 10 | 1 | 0.655 | 1.26 | 0.0E+00 | 0.0E+00 |
| Pold4 | 10 | 0.973 | 0.344 | 1.24 | 0.0E+00 | 0.0E+00 |
| Mzb1 | 10 | 0.97 | 0.221 | 1.23 | 0.0E+00 | 0.0E+00 |
| H2-Eb1 | 10 | 0.999 | 0.43 | 1.16 | 0.0E+00 | 0.0E+00 |
| H2-DMb2 | 10 | 0.971 | 0.276 | 1.15 | 0.0E+00 | 0.0E+00 |
| Aicda | 10 | 0.833 | 0.056 | 1.15 | 0.0E+00 | 0.0E+00 |
| H2-Aa | 10 | 0.999 | 0.472 | 1.14 | 0.0E+00 | 0.0E+00 |
| Tnfrsf13c | 10 | 0.864 | 0.149 | 1.11 | 0.0E+00 | 0.0E+00 |
| Sypl | 10 | 0.903 | 0.205 | 1.11 | 0.0E+00 | 0.0E+00 |
| Crip1 | 10 | 0.982 | 0.579 | 1.10 | 0.0E+00 | 0.0E+00 |
| Rhoh | 10 | 0.96 | 0.479 | 1.04 | 0.0E+00 | 0.0E+00 |
| H2-Ab1 | 10 | 0.995 | 0.505 | 1.02 | 0.0E+00 | 0.0E+00 |
| Eaf2 | 10 | 0.798 | 0.067 | 1.00 | 0.0E+00 | 0.0E+00 |
| Ifi30 | 10 | 0.922 | 0.288 | 1.00 | 0.0E+00 | 0.0E+00 |
| Nap1l1 | 10 | 0.96 | 0.434 | 0.99 | 0.0E+00 | 0.0E+00 |
| Arpc5l | 10 | 0.944 | 0.396 | 0.99 | 0.0E+00 | 0.0E+00 |
| Scimp | 10 | 0.855 | 0.141 | 0.97 | 0.0E+00 | 0.0E+00 |
| Cd37 | 10 | 0.965 | 0.569 | 0.95 | 0.0E+00 | 0.0E+00 |
| Apoe | 10 | 0.81 | 0.211 | 0.95 | 0.0E+00 | 0.0E+00 |
| H2-DMa | 10 | 0.934 | 0.319 | 0.94 | 0.0E+00 | 0.0E+00 |
| Iglc1 | 10 | 0.827 | 0.204 | 0.93 | 0.0E+00 | 0.0E+00 |
| Basp1 | 10 | 0.821 | 0.135 | 0.93 | 0.0E+00 | 0.0E+00 |
| Pou2af1 | 10 | 0.84 | 0.131 | 0.91 | 0.0E+00 | 0.0E+00 |
| Smim14 | 10 | 0.95 | 0.468 | 0.91 | 0.0E+00 | 0.0E+00 |
| Hmces | 10 | 0.8 | 0.138 | 0.90 | 0.0E+00 | 0.0E+00 |
| Vpreb3 | 10 | 0.743 | 0.136 | 0.88 | 0.0E+00 | 0.0E+00 |
| Bfsp2 | 10 | 0.649 | 0.063 | 0.83 | 0.0E+00 | 0.0E+00 |
| Grb2 | 10 | 0.877 | 0.35 | 0.83 | 0.0E+00 | 0.0E+00 |
| Mef2c | 10 | 0.922 | 0.276 | 0.81 | 0.0E+00 | 0.0E+00 |
| Klhl6 | 10 | 0.817 | 0.245 | 0.81 | 0.0E+00 | 0.0E+00 |
| Zbtb20 | 10 | 0.829 | 0.314 | 0.78 | 0.0E+00 | 0.0E+00 |
| Neil1 | 10 | 0.705 | 0.082 | 0.78 | 0.0E+00 | 0.0E+00 |
| Erp44 | 10 | 0.801 | 0.262 | 0.78 | 0.0E+00 | 0.0E+00 |
| Glrx3 | 10 | 0.828 | 0.324 | 0.77 | 0.0E+00 | 0.0E+00 |
| Siglecg | 10 | 0.794 | 0.164 | 0.77 | 0.0E+00 | 0.0E+00 |
| H3f3a | 10 | 0.999 | 0.926 | 0.77 | 0.0E+00 | 0.0E+00 |
| Iglc2 | 10 | 0.825 | 0.21 | 0.76 | 0.0E+00 | 0.0E+00 |
| Hvcn1 | 10 | 0.747 | 0.263 | 0.76 | 0.0E+00 | 0.0E+00 |
| Smagp | 10 | 0.691 | 0.064 | 0.76 | 0.0E+00 | 0.0E+00 |
| Gcsam | 10 | 0.674 | 0.065 | 0.75 | 0.0E+00 | 0.0E+00 |
| Cd19 | 10 | 0.745 | 0.109 | 0.74 | 0.0E+00 | 0.0E+00 |
| Pou2f2 | 10 | 0.792 | 0.215 | 0.74 | 0.0E+00 | 0.0E+00 |
| Cfl1 | 10 | 0.998 | 0.94 | 0.73 | 0.0E+00 | 0.0E+00 |
| Lrmp | 10 | 0.797 | 0.252 | 0.73 | 0.0E+00 | 0.0E+00 |
| Mif4gd | 10 | 0.81 | 0.305 | 0.73 | 0.0E+00 | 0.0E+00 |
| Cd24a | 10 | 0.842 | 0.195 | 0.73 | 0.0E+00 | 0.0E+00 |
| Lat2 | 10 | 0.763 | 0.194 | 0.71 | 0.0E+00 | 0.0E+00 |
| H2-Ob | 10 | 0.741 | 0.145 | 0.70 | 0.0E+00 | 0.0E+00 |
| Hmgn1 | 10 | 0.924 | 0.489 | 0.69 | 0.0E+00 | 0.0E+00 |
| Ube2d2a | 10 | 0.897 | 0.564 | 0.69 | 0.0E+00 | 0.0E+00 |
| Coro1a | 10 | 0.989 | 0.879 | 0.68 | 0.0E+00 | 0.0E+00 |
| S1pr2 | 10 | 0.602 | 0.053 | 0.68 | 0.0E+00 | 0.0E+00 |
| Cd22 | 10 | 0.64 | 0.08 | 0.68 | 0.0E+00 | 0.0E+00 |
| Plekho1 | 10 | 0.731 | 0.155 | 0.68 | 0.0E+00 | 0.0E+00 |
| Apobec1 | 10 | 0.68 | 0.098 | 0.66 | 0.0E+00 | 0.0E+00 |
| Pkig | 10 | 0.788 | 0.239 | 0.66 | 0.0E+00 | 0.0E+00 |
| Dynll1 | 10 | 0.958 | 0.682 | 0.66 | 0.0E+00 | 0.0E+00 |
| Rftn1 | 10 | 0.768 | 0.266 | 0.64 | 0.0E+00 | 0.0E+00 |
| Clic1 | 10 | 0.958 | 0.718 | 0.64 | 0.0E+00 | 0.0E+00 |
| Arpc2 | 10 | 0.985 | 0.831 | 0.64 | 0.0E+00 | 0.0E+00 |
| Lpxn | 10 | 0.742 | 0.25 | 0.63 | 0.0E+00 | 0.0E+00 |
| Trp53i11 | 10 | 0.64 | 0.156 | 0.63 | 0.0E+00 | 0.0E+00 |
| Spib | 10 | 0.706 | 0.166 | 0.62 | 0.0E+00 | 0.0E+00 |
| Swap70 | 10 | 0.64 | 0.136 | 0.62 | 0.0E+00 | 0.0E+00 |
| Fcmr | 10 | 0.732 | 0.145 | 0.61 | 0.0E+00 | 0.0E+00 |
| Lipc | 10 | 0.523 | 0.039 | 0.60 | 0.0E+00 | 0.0E+00 |
| Iglc3 | 10 | 0.89 | 0.252 | 0.59 | 0.0E+00 | 0.0E+00 |
| Apitd1 | 10 | 0.641 | 0.093 | 0.58 | 0.0E+00 | 0.0E+00 |
| Snx29 | 10 | 0.579 | 0.105 | 0.57 | 0.0E+00 | 0.0E+00 |
| Sh2b2 | 10 | 0.575 | 0.069 | 0.55 | 0.0E+00 | 0.0E+00 |
| Blnk | 10 | 0.676 | 0.171 | 0.55 | 0.0E+00 | 0.0E+00 |
| Mtf2 | 10 | 0.631 | 0.152 | 0.55 | 0.0E+00 | 0.0E+00 |
| Dok3 | 10 | 0.602 | 0.108 | 0.55 | 0.0E+00 | 0.0E+00 |
| Mtmr14 | 10 | 0.563 | 0.109 | 0.53 | 0.0E+00 | 0.0E+00 |
| Dcaf12 | 10 | 0.632 | 0.17 | 0.52 | 0.0E+00 | 0.0E+00 |
| Lyl1 | 10 | 0.529 | 0.083 | 0.50 | 0.0E+00 | 0.0E+00 |
| Myl4 | 10 | 0.443 | 0.066 | 0.50 | 0.0E+00 | 0.0E+00 |
| Slc25a19 | 10 | 0.533 | 0.108 | 0.50 | 0.0E+00 | 0.0E+00 |
| Fcrl1 | 10 | 0.552 | 0.098 | 0.49 | 0.0E+00 | 0.0E+00 |
| Srpk3 | 10 | 0.472 | 0.048 | 0.47 | 0.0E+00 | 0.0E+00 |
| Nuggc | 10 | 0.458 | 0.033 | 0.46 | 0.0E+00 | 0.0E+00 |
| 4930597A21Rik | 10 | 0.395 | 0.049 | 0.45 | 0.0E+00 | 0.0E+00 |
| Ildr1 | 10 | 0.404 | 0.035 | 0.42 | 0.0E+00 | 0.0E+00 |
| Rab30 | 10 | 0.427 | 0.062 | 0.41 | 0.0E+00 | 0.0E+00 |
| Cd86 | 10 | 0.448 | 0.068 | 0.40 | 0.0E+00 | 0.0E+00 |
| Arhgap8 | 10 | 0.397 | 0.032 | 0.39 | 0.0E+00 | 0.0E+00 |
| Csrp2 | 10 | 0.414 | 0.059 | 0.38 | 0.0E+00 | 0.0E+00 |
| Mbd4 | 10 | 0.367 | 0.041 | 0.35 | 0.0E+00 | 0.0E+00 |
| Tmem243 | 10 | 0.732 | 0.287 | 0.59 | 1.4E−312 | 2.9E−308 |
| Anp32b | 10 | 0.927 | 0.541 | 0.62 | 1.4E−310 | 2.8E−306 |
| 1-Mar | 10 | 0.426 | 0.068 | 0.39 | 2.5E−307 | 4.9E−303 |
| Efnb1 | 10 | 0.333 | 0.038 | 0.33 | 4.7E−307 | 9.0E−303 |
| Cd83 | 10 | 0.773 | 0.277 | 0.76 | 1.9E−305 | 3.7E−301 |
| Top1 | 10 | 0.831 | 0.389 | 0.63 | 1.1E−303 | 2.1E−299 |
| Il4i1 | 10 | 0.417 | 0.065 | 0.62 | 1.0E−298 | 1.9E−294 |
| Il21r | 10 | 0.655 | 0.22 | 0.56 | 4.5E−298 | 8.7E−294 |
| Gh | 10 | 0.26 | 0.019 | 0.32 | 3.1E−297 | 6.0E−293 |
| Mybl1 | 10 | 0.274 | 0.024 | 0.28 | 2.3E−292 | 4.5E−288 |
| Eef2 | 10 | 0.998 | 0.945 | 0.64 | 1.6E−291 | 3.1E−287 |
| Blk | 10 | 0.474 | 0.09 | 0.41 | 1.8E−289 | 3.5E−285 |
| Ublcp1 | 10 | 0.536 | 0.124 | 0.45 | 1.0E−288 | 1.9E−284 |
| Bcar3 | 10 | 0.411 | 0.066 | 0.38 | 3.7E−287 | 7.2E−283 |
| Pxk | 10 | 0.515 | 0.122 | 0.44 | 1.5E−286 | 2.9E−282 |
| Cd81 | 10 | 0.722 | 0.241 | 0.53 | 3.7E−286 | 7.1E−282 |
| Ctsh | 10 | 0.816 | 0.29 | 0.31 | 4.3E−284 | 8.3E−280 |
| Zfos1 | 10 | 0.755 | 0.342 | 0.61 | 3.3E−283 | 6.3E−279 |
| Eif3h | 10 | 0.971 | 0.809 | 0.58 | 8.7E−283 | 1.7E−278 |
| Dck | 10 | 0.456 | 0.085 | 0.38 | 1.7E−282 | 3.3E−278 |
| Csk | 10 | 0.838 | 0.455 | 0.59 | 4.7E−282 | 9.0E−278 |
| Bach2 | 10 | 0.449 | 0.086 | 0.40 | 2.3E−276 | 4.3E−272 |
| Cd180 | 10 | 0.52 | 0.117 | 0.47 | 1.5E−274 | 2.9E−270 |
| Xcl1 | 11 | 0.908 | 0.077 | 1.72 | 0.0E+00 | 0.0E+00 |
| Ccl5 | 11 | 0.973 | 0.2 | 1.58 | 0.0E+00 | 0.0E+00 |
| Ncr1 | 11 | 0.952 | 0.143 | 1.39 | 0.0E+00 | 0.0E+00 |
| Nkg7 | 11 | 0.964 | 0.233 | 1.20 | 0.0E+00 | 0.0E+00 |
| AW112010 | 11 | 0.997 | 0.578 | 1.19 | 0.0E+00 | 0.0E+00 |
| Klrd1 | 11 | 0.853 | 0.154 | 1.00 | 0.0E+00 | 0.0E+00 |
| Ctsw | 11 | 0.906 | 0.235 | 0.97 | 0.0E+00 | 0.0E+00 |
| Klrk1 | 11 | 0.88 | 0.184 | 0.95 | 0.0E+00 | 0.0E+00 |
| Klrc1 | 11 | 0.595 | 0.055 | 0.92 | 0.0E+00 | 0.0E+00 |
| Cd7 | 11 | 0.924 | 0.366 | 0.90 | 0.0E+00 | 0.0E+00 |
| Gimap4 | 11 | 0.944 | 0.46 | 0.89 | 0.0E+00 | 0.0E+00 |
| Ikzf2 | 11 | 0.759 | 0.211 | 0.87 | 0.0E+00 | 0.0E+00 |
| Il2rb | 11 | 0.779 | 0.207 | 0.78 | 0.0E+00 | 0.0E+00 |
| Cd160 | 11 | 0.681 | 0.121 | 0.75 | 0.0E+00 | 0.0E+00 |
| Gimap3 | 11 | 0.928 | 0.509 | 0.74 | 0.0E+00 | 0.0E+00 |
| Fcer1g | 11 | 0.966 | 0.356 | 0.69 | 0.0E+00 | 0.0E+00 |
| Fasl | 11 | 0.598 | 0.071 | 0.61 | 0.0E+00 | 0.0E+00 |
| Klrc2 | 11 | 0.484 | 0.045 | 0.60 | 0.0E+00 | 0.0E+00 |
| Klrb1f | 11 | 0.579 | 0.095 | 0.60 | 0.0E+00 | 0.0E+00 |
| Klrb1a | 11 | 0.432 | 0.03 | 0.57 | 0.0E+00 | 0.0E+00 |
| Hopx | 11 | 0.493 | 0.044 | 0.51 | 0.0E+00 | 0.0E+00 |
| Tyrobp | 11 | 0.945 | 0.292 | 0.38 | 0.0E+00 | 0.0E+00 |
| Serpinb6b | 11 | 0.642 | 0.152 | 0.72 | 7.4E−288 | 1.4E−283 |
| Car2 | 11 | 0.783 | 0.243 | 0.82 | 3.9E−283 | 7.4E−279 |
| Bcl2a1d | 11 | 0.643 | 0.165 | 0.61 | 1.2E−282 | 2.3E−278 |
| Hcst | 11 | 0.921 | 0.522 | 0.69 | 3.4E−276 | 6.5E−272 |
| Klrb1c | 11 | 0.484 | 0.063 | 0.56 | 1.2E−275 | 2.3E−271 |
| Hes1 | 12 | 0.772 | 0.06 | 2.21 | 0.0E+00 | 0.0E+00 |
| Lmo4 | 12 | 0.988 | 0.317 | 2.02 | 0.0E+00 | 0.0E+00 |
| Hilpda | 12 | 0.86 | 0.158 | 1.90 | 0.0E+00 | 0.0E+00 |
| Calca | 12 | 0.616 | 0.014 | 1.87 | 0.0E+00 | 0.0E+00 |
| Il13 | 12 | 0.444 | 0.01 | 1.68 | 0.0E+00 | 0.0E+00 |
| Gata3 | 12 | 0.946 | 0.128 | 1.68 | 0.0E+00 | 0.0E+00 |
| Hs3st1 | 12 | 0.846 | 0.082 | 1.55 | 0.0E+00 | 0.0E+00 |
| Areg | 12 | 0.448 | 0.018 | 1.41 | 0.0E+00 | 0.0E+00 |
| Tcrg-C1 | 12 | 0.786 | 0.108 | 1.34 | 0.0E+00 | 0.0E+00 |
| Junb | 12 | 0.989 | 0.749 | 1.28 | 0.0E+00 | 0.0E+00 |
| Nfkb1 | 12 | 0.834 | 0.322 | 1.22 | 0.0E+00 | 0.0E+00 |
| Nr4a1 | 12 | 0.894 | 0.376 | 1.16 | 0.0E+00 | 0.0E+00 |
| Il4 | 12 | 0.546 | 0.018 | 1.13 | 0.0E+00 | 0.0E+00 |
| Furin | 12 | 0.662 | 0.153 | 1.11 | 0.0E+00 | 0.0E+00 |
| Nfkbiz | 12 | 0.813 | 0.236 | 1.03 | 0.0E+00 | 0.0E+00 |
| Nrip1 | 12 | 0.878 | 0.319 | 0.99 | 0.0E+00 | 0.0E+00 |
| Dnaja1 | 12 | 0.903 | 0.61 | 0.98 | 0.0E+00 | 0.0E+00 |
| Samsn1 | 12 | 0.891 | 0.328 | 0.95 | 0.0E+00 | 0.0E+00 |
| Spty2d1 | 12 | 0.618 | 0.157 | 0.94 | 0.0E+00 | 0.0E+00 |
| Sptssa | 12 | 0.903 | 0.481 | 0.94 | 0.0E+00 | 0.0E+00 |
| Rora | 12 | 0.788 | 0.189 | 0.92 | 0.0E+00 | 0.0E+00 |
| Zfp36l1 | 12 | 0.956 | 0.604 | 0.92 | 0.0E+00 | 0.0E+00 |
| Emb | 12 | 0.926 | 0.443 | 0.89 | 0.0E+00 | 0.0E+00 |
| Gpr65 | 12 | 0.758 | 0.174 | 0.83 | 0.0E+00 | 0.0E+00 |
| H3f3b | 12 | 0.997 | 0.953 | 0.76 | 0.0E+00 | 0.0E+00 |
| Il17rb | 12 | 0.643 | 0.013 | 0.76 | 0.0E+00 | 0.0E+00 |
| Deptor | 12 | 0.59 | 0.015 | 0.74 | 0.0E+00 | 0.0E+00 |
| 4930523C07Rik | 12 | 0.769 | 0.416 | 0.72 | 0.0E+00 | 0.0E+00 |
| Frmd4b | 12 | 0.6 | 0.072 | 0.68 | 0.0E+00 | 0.0E+00 |
| Itk | 12 | 0.613 | 0.173 | 0.65 | 0.0E+00 | 0.0E+00 |
| Epas1 | 12 | 0.605 | 0.055 | 0.64 | 0.0E+00 | 0.0E+00 |
| 1810011H11Rik | 12 | 0.536 | 0.041 | 0.59 | 0.0E+00 | 0.0E+00 |
| Homer2 | 12 | 0.446 | 0.007 | 0.54 | 0.0E+00 | 0.0E+00 |
| AA467197 | 12 | 0.416 | 0.016 | 0.51 | 0.0E+00 | 0.0E+00 |
| Ret | 12 | 0.493 | 0.041 | 0.50 | 0.0E+00 | 0.0E+00 |
| Ptpn13 | 12 | 0.431 | 0.009 | 0.50 | 0.0E+00 | 0.0E+00 |
| Rxrg | 12 | 0.232 | 0.001 | 0.26 | 2.1E−305 | 4.1E−301 |
| Icos | 12 | 0.637 | 0.135 | 0.70 | 2.1E−301 | 4.0E−297 |
| Ccr4 | 12 | 0.304 | 0.009 | 0.37 | 5.9E−293 | 1.1E−288 |
| Btg2 | 12 | 0.959 | 0.72 | 0.94 | 3.7E−291 | 7.0E−287 |
| Rgs2 | 12 | 0.835 | 0.498 | 1.02 | 1.8E−283 | 3.5E−279 |
| Vps37b | 12 | 0.812 | 0.408 | 1.03 | 1.9E−282 | 3.7E−278 |
| S100a4 | 14 | 0.928 | 0.154 | 2.33 | 0.0E+00 | 0.0E+00 |
| S100a11 | 14 | 0.983 | 0.494 | 2.33 | 0.0E+00 | 0.0E+00 |
| Mdh2 | 14 | 0.973 | 0.515 | 2.27 | 0.0E+00 | 0.0E+00 |
| S100a6 | 14 | 0.953 | 0.191 | 2.25 | 0.0E+00 | 0.0E+00 |
| Atox1 | 14 | 0.962 | 0.498 | 2.08 | 0.0E+00 | 0.0E+00 |
| Cfp | 14 | 0.908 | 0.056 | 1.98 | 0.0E+00 | 0.0E+00 |
| Napsa | 14 | 0.981 | 0.271 | 1.83 | 0.0E+00 | 0.0E+00 |
| Cst3 | 14 | 0.989 | 0.577 | 1.81 | 0.0E+00 | 0.0E+00 |
| Bcl2a1b | 14 | 0.883 | 0.173 | 1.78 | 0.0E+00 | 0.0E+00 |
| Gm2a | 14 | 0.964 | 0.33 | 1.75 | 0.0E+00 | 0.0E+00 |
| Gpx1 | 14 | 0.995 | 0.58 | 1.72 | 0.0E+00 | 0.0E+00 |
| Vim | 14 | 0.925 | 0.324 | 1.67 | 0.0E+00 | 0.0E+00 |
| Rogdi | 14 | 0.917 | 0.093 | 1.67 | 0.0E+00 | 0.0E+00 |
| Spi1 | 14 | 0.934 | 0.146 | 1.66 | 0.0E+00 | 0.0E+00 |
| Taldo1 | 14 | 0.962 | 0.506 | 1.61 | 0.0E+00 | 0.0E+00 |
| Bcl2a1d | 14 | 0.901 | 0.165 | 1.59 | 0.0E+00 | 0.0E+00 |
| Tbc1d4 | 14 | 0.793 | 0.064 | 1.58 | 0.0E+00 | 0.0E+00 |
| Gsn | 14 | 0.909 | 0.181 | 1.55 | 0.0E+00 | 0.0E+00 |
| Lsp1 | 14 | 0.992 | 0.679 | 1.46 | 0.0E+00 | 0.0E+00 |
| Grasp | 14 | 0.723 | 0.059 | 1.44 | 0.0E+00 | 0.0E+00 |
| Vasp | 14 | 0.964 | 0.614 | 1.43 | 0.0E+00 | 0.0E+00 |
| Ppp1r14a | 14 | 0.718 | 0.009 | 1.40 | 0.0E+00 | 0.0E+00 |
| Pglyrp1 | 14 | 0.786 | 0.116 | 1.39 | 0.0E+00 | 0.0E+00 |
| Ms4a6c | 14 | 0.861 | 0.085 | 1.36 | 0.0E+00 | 0.0E+00 |
| Il1r2 | 14 | 0.603 | 0.016 | 1.34 | 0.0E+00 | 0.0E+00 |
| Plbd1 | 14 | 0.833 | 0.051 | 1.33 | 0.0E+00 | 0.0E+00 |
| Ywhah | 14 | 0.915 | 0.429 | 1.29 | 0.0E+00 | 0.0E+00 |
| Avpi1 | 14 | 0.787 | 0.086 | 1.26 | 0.0E+00 | 0.0E+00 |
| Efhd2 | 14 | 0.839 | 0.215 | 1.10 | 0.0E+00 | 0.0E+00 |
| Tctex1d2 | 14 | 0.792 | 0.104 | 1.07 | 0.0E+00 | 0.0E+00 |
| Snx20 | 14 | 0.801 | 0.19 | 1.04 | 0.0E+00 | 0.0E+00 |
| M6pr | 14 | 0.854 | 0.333 | 1.03 | 0.0E+00 | 0.0E+00 |
| Cyp4f16 | 14 | 0.671 | 0.075 | 1.02 | 0.0E+00 | 0.0E+00 |
| Fam105a | 14 | 0.776 | 0.194 | 0.98 | 0.0E+00 | 0.0E+00 |
| Pak1 | 14 | 0.598 | 0.015 | 0.80 | 0.0E+00 | 0.0E+00 |
| Clec4a2 | 14 | 0.512 | 0.009 | 0.77 | 3.5E−303 | 6.7E−299 |
| 2900052N01Rik | 14 | 0.433 | 0.004 | 0.63 | 5.0E−302 | 9.6E−298 |
| Bcl2a1a | 14 | 0.66 | 0.031 | 1.28 | 6.0E−298 | 1.1E−293 |
| Arpc1b | 14 | 0.995 | 0.846 | 1.35 | 8.5E−295 | 1.6E−290 |
| Ostf1 | 14 | 0.945 | 0.586 | 1.11 | 3.3E−293 | 6.3E−289 |
| H2-Eb1 | 14 | 0.994 | 0.447 | 2.18 | 7.1E−289 | 1.4E−284 |
| Btg2 | 14 | 0.984 | 0.722 | 1.34 | 6.3E−288 | 1.2E−283 |
| Rel | 14 | 0.826 | 0.204 | 1.14 | 1.3E−286 | 2.5E−282 |
| Wdfy4 | 14 | 0.742 | 0.062 | 0.92 | 2.6E−285 | 5.0E−281 |
| Mkrn1 | 14 | 0.833 | 0.344 | 0.93 | 3.3E−281 | 6.4E−277 |
| Anpep | 14 | 0.51 | 0.012 | 0.65 | l.0E−277 | 2.0E−273 |
| Ccnd1 | 14 | 0.651 | 0.052 | 1.13 | 2.8E−275 | 5.4E−271 |
| H2-Ab1 | 14 | 0.995 | 0.519 | 2.12 | 1.7E−271 | 3.2E−267 |
| Cd7 | 17 | 0.988 | 0.375 | 2.02 | 0.0E+00 | 0.0E+00 |
| Cd160 | 17 | 0.924 | 0.128 | 1.50 | 0.0E+00 | 0.0E+00 |
| Dapk2 | 17 | 0.786 | 0.067 | 1.16 | 0.0E+00 | 0.0E+00 |
| Ctsw | 17 | 0.956 | 0.246 | 1.26 | 3.1E−308 | 6.0E−304 |
| Cd3g | 17 | 0.83 | 0.377 | 1.58 | 8.6E−307 | 1.7E−302 |
| Igha | 20 | 0.924 | 0.291 | 6.73 | 0.0E+00 | 0.0E+00 |
| Igkc | 20 | 0.996 | 0.706 | 5.98 | 0.0E+00 | 0.0E+00 |
| Jchain | 20 | 0.996 | 0.239 | 5.84 | 0.0E+00 | 0.0E+00 |
| Iglv1 | 20 | 0.75 | 0.034 | 3.48 | 0.0E+00 | 0.0E+00 |
| Mzb1 | 20 | 0.962 | 0.251 | 3.03 | 0.0E+00 | 0.0E+00 |
| Txndc5 | 20 | 0.873 | 0.188 | 2.71 | 0.0E+00 | 0.0E+00 |
| Hsp90b1 | 20 | 0.907 | 0.362 | 2.68 | 0.0E+00 | 0.0E+00 |
| Ssr4 | 20 | 0.919 | 0.537 | 2.39 | 0.0E+00 | 0.0E+00 |
| Sec11c | 20 | 0.928 | 0.538 | 2.19 | 0.0E+00 | 0.0E+00 |
| Pdia4 | 20 | 0.839 | 0.186 | 2.11 | 0.0E+00 | 0.0E+00 |
| Xbp1 | 20 | 0.86 | 0.268 | 2.07 | 0.0E+00 | 0.0E+00 |
| Edem1 | 20 | 0.843 | 0.167 | 2.04 | 0.0E+00 | 0.0E+00 |
| Manf | 20 | 0.898 | 0.459 | 1.99 | 0.0E+00 | 0.0E+00 |
| H13 | 20 | 0.869 | 0.301 | 1.94 | 0.0E+00 | 0.0E+00 |
| Spcs1 | 20 | 0.886 | 0.484 | 1.78 | 0.0E+00 | 0.0E+00 |
| Ppib | 20 | 0.877 | 0.627 | 1.76 | 0.0E+00 | 0.0E+00 |
| Sdf2l1 | 20 | 0.843 | 0.194 | 1.76 | 0.0E+00 | 0.0E+00 |
| Sec61b | 20 | 0.915 | 0.688 | 1.75 | 0.0E+00 | 0.0E+00 |
| Pdia6 | 20 | 0.852 | 0.332 | 1.73 | 0.0E+00 | 0.0E+00 |
| Derl3 | 20 | 0.805 | 0.017 | 1.70 | 0.0E+00 | 0.0E+00 |
| Hspa5 | 20 | 0.86 | 0.503 | 1.68 | 0.0E+00 | 0.0E+00 |
| Calr | 20 | 0.881 | 0.524 | 1.67 | 0.0E+00 | 0.0E+00 |
| Fkbp2 | 20 | 0.835 | 0.201 | 1.64 | 0.0E+00 | 0.0E+00 |
| Rexo2 | 20 | 0.839 | 0.387 | 1.63 | 0.0E+00 | 0.0E+00 |
| Edem2 | 20 | 0.835 | 0.174 | 1.62 | 0.0E+00 | 0.0E+00 |
| Prdx4 | 20 | 0.771 | 0.085 | 1.60 | 0.0E+00 | 0.0E+00 |
| Spcs2 | 20 | 0.898 | 0.599 | 1.60 | 0.0E+00 | 0.0E+00 |
| Krtcap2 | 20 | 0.898 | 0.556 | 1.58 | 0.0E+00 | 0.0E+00 |
| Creld2 | 20 | 0.767 | 0.128 | 1.55 | 0.0E+00 | 0.0E+00 |
| Trp53inp1 | 20 | 0.746 | 0.115 | 1.49 | 0.0E+00 | 0.0E+00 |
| Txndc11 | 20 | 0.792 | 0.136 | 1.46 | 0.0E+00 | 0.0E+00 |
| Selk | 20 | 0.877 | 0.596 | 1.41 | 0.0E+00 | 0.0E+00 |
| Lman1 | 20 | 0.788 | 0.098 | 1.40 | 0.0E+00 | 0.0E+00 |
| Dnajc3 | 20 | 0.784 | 0.268 | 1.38 | 0.0E+00 | 0.0E+00 |
| Dnajb11 | 20 | 0.78 | 0.252 | 1.38 | 0.0E+00 | 0.0E+00 |
| Srp9 | 20 | 0.869 | 0.534 | 1.34 | 0.0E+00 | 0.0E+00 |
| Ssr2 | 20 | 0.805 | 0.325 | 1.33 | 0.0E+00 | 0.0E+00 |
| Tmed10 | 20 | 0.86 | 0.388 | 1.30 | 0.0E+00 | 0.0E+00 |
| Ddost | 20 | 0.788 | 0.314 | 1.29 | 0.0E+00 | 0.0E+00 |
| Ckap4 | 20 | 0.754 | 0.012 | 1.28 | 0.0E+00 | 0.0E+00 |
| Ndufa1 | 20 | 0.814 | 0.502 | 1.27 | 0.0E+00 | 0.0E+00 |
| Ssr3 | 20 | 0.754 | 0.207 | 1.24 | 0.0E+00 | 0.0E+00 |
| Clptm1l | 20 | 0.763 | 0.227 | 1.15 | 0.0E+00 | 0.0E+00 |
| Ift20 | 20 | 0.763 | 0.283 | 1.14 | 0.0E+00 | 0.0E+00 |
| Tmem248 | 20 | 0.703 | 0.148 | 0.92 | 0.0E+00 | 0.0E+00 |
| Tnfrsf17 | 20 | 0.61 | 0.001 | 1.41 | 1.9E−307 | 3.6E−303 |
| Spcs3 | 20 | 0.758 | 0.185 | 1.12 | 9.6E−307 | 1.9E−302 |
| Cope | 20 | 0.826 | 0.417 | 1.15 | 2.0E−303 | 3.8E−299 |
| Fkbp11 | 20 | 0.682 | 0.005 | 1.36 | 3.1E−301 | 5.9E−297 |
| Ccr10 | 20 | 0.602 | 0.001 | 1.39 | 1.6E−297 | 3.2E−293 |
| Serp1 | 20 | 0.898 | 0.545 | 1.64 | 1.1E−290 | 2.1E−286 |
| Derl1 | 20 | 0.733 | 0.292 | 1.00 | 1.4E−289 | 2.7E−285 |
| Sdc1 | 20 | 0.729 | 0.062 | 1.25 | 1.1E−283 | 2.1E−279 |
| Rpn2 | 20 | 0.75 | 0.306 | 1.13 | 4.4E−280 | 8.4E−276 |
| Reep5 | 20 | 0.826 | 0.438 | 1.30 | 3.1E−279 | 6.0E−275 |
| Slc35b1 | 20 | 0.674 | 0.133 | 0.96 | 3.3E−279 | 6.3E−275 |
| Sec61g | 20 | 0.881 | 0.759 | 1.35 | 1.3E−277 | 2.5E−273 |
| Iglc2 | 20 | 0.907 | 0.234 | 3.07 | 1.0E−274 | 2.0E−270 |
| Cst3 | 21 | 1 | 0.582 | 4.06 | 0.0E+00 | 0.0E+00 |
| Ppt1 | 21 | 0.897 | 0.177 | 2.41 | 0.0E+00 | 0.0E+00 |
| Vim | 21 | 0.979 | 0.331 | 2.29 | 0.0E+00 | 0.0E+00 |
| H2-DMb1 | 21 | 0.987 | 0.164 | 2.27 | 0.0E+00 | 0.0E+00 |
| Naaa | 21 | 0.838 | 0.034 | 2.27 | 0.0E+00 | 0.0E+00 |
| Atox1 | 21 | 0.983 | 0.503 | 2.11 | 0.0E+00 | 0.0E+00 |
| Fuca1 | 21 | 0.859 | 0.231 | 1.38 | 0.0E+00 | 0.0E+00 |
| Cxx1a | 21 | 0.778 | 0.09 | 1.28 | 4.7E−286 | 9.0E−282 |
| Psmb9 | 21 | 0.944 | 0.441 | 1.57 | 3.6E−280 | 7.0E−276 |
| Fgd2 | 21 | 0.876 | 0.051 | 1.29 | 2.8E−277 | 5.3E−273 |
| Aif1 | 21 | 0.915 | 0.05 | 1.96 | 2.5E−274 | 4.8E−270 |
| Lyz2 | 22 | 0.991 | 0.073 | 4.40 | 0.0E+00 | 0.0E+00 |
| Ifitm3 | 22 | 0.961 | 0.058 | 2.97 | 0.0E+00 | 0.0E+00 |
| Cebpb | 22 | 0.953 | 0.256 | 2.44 | 0.0E+00 | 0.0E+00 |
| Lst1 | 22 | 0.953 | 0.146 | 2.34 | 0.0E+00 | 0.0E+00 |
| Psap | 22 | 1 | 0.531 | 2.07 | 0.0E+00 | 0.0E+00 |
| Ms4a6c | 22 | 0.935 | 0.093 | 2.01 | 0.0E+00 | 0.0E+00 |
| Ctss | 22 | 0.957 | 0.391 | 1.80 | 0.0E+00 | 0.0E+00 |
| Mafb | 22 | 0.75 | 0.008 | 1.77 | 0.0E+00 | 0.0E+00 |
| Msrb1 | 22 | 0.875 | 0.206 | 1.75 | 0.0E+00 | 0.0E+00 |
| Sat1 | 22 | 0.953 | 0.458 | 1.67 | 0.0E+00 | 0.0E+00 |
| Prdx5 | 22 | 0.901 | 0.466 | 1.59 | 0.0E+00 | 0.0E+00 |
| Csf1r | 22 | 0.832 | 0.007 | 1.31 | 0.0E+00 | 0.0E+00 |
| Clec4a3 | 22 | 0.776 | 0.005 | 1.27 | 0.0E+00 | 0.0E+00 |
| Ccl6 | 22 | 0.836 | 0.019 | 1.92 | 3.9E−305 | 7.5E−301 |
| Ccl9 | 22 | 0.728 | 0.007 | 1.71 | 5.0E−293 | 9.6E−289 |
| Sirpb1b | 22 | 0.724 | 0.006 | 1.05 | 5.7E−293 | 1.1E−288 |
| Sirpb1c | 22 | 0.741 | 0.008 | 1.08 | 4.9E−289 | 9.4E−285 |
| Lamp1 | 22 | 0.931 | 0.485 | 1.34 | 6.3E−288 | 1.2E−283 |
| Clec4a1 | 22 | 0.69 | 0.006 | 1.00 | 1.4E−282 | 2.7E−278 |
| Fscn1 | 24 | 0.883 | 0.029 | 2.62 | 0.0E+00 | 0.0E+00 |
| Ccr7 | 24 | 0.906 | 0.302 | 2.44 | 0.0E+00 | 0.0E+00 |
| Tbc1d4 | 24 | 0.928 | 0.071 | 2.07 | 0.0E+00 | 0.0E+00 |
| Tmem123 | 24 | 0.924 | 0.376 | 1.83 | 0.0E+00 | 0.0E+00 |
| Marcks | 24 | 0.919 | 0.076 | 1.66 | 0.0E+00 | 0.0E+00 |
| Tspan3 | 24 | 0.78 | 0.09 | 1.54 | 0.0E+00 | 0.0E+00 |
| Lamp1 | 24 | 0.933 | 0.485 | 1.48 | 0.0E+00 | 0.0E+00 |
| Ogfrl1 | 24 | 0.861 | 0.094 | 1.46 | 0.0E+00 | 0.0E+00 |
| Iscu | 24 | 0.915 | 0.32 | 1.44 | 0.0E+00 | 0.0E+00 |
| Relb | 24 | 0.924 | 0.189 | 1.43 | 0.0E+00 | 0.0E+00 |
| Cacnb3 | 24 | 0.771 | 0.004 | 1.14 | 0.0E+00 | 0.0E+00 |
| Birc2 | 24 | 0.78 | 0.101 | 1.09 | 0.0E+00 | 0.0E+00 |
| Tmcc3 | 24 | 0.812 | 0.049 | 1.19 | 4.7E−307 | 9.1E−303 |
| Marcksl1 | 24 | 0.951 | 0.177 | 2.06 | 1.0E−297 | 1.9E−293 |
| Traf1 | 24 | 0.906 | 0.328 | 1.53 | 2.2E−294 | 4.3E−290 |
| Cxcl16 | 24 | 0.839 | 0.026 | 1.73 | 1.3E−285 | 2.6E−281 |
| Igfbp7 | 25 | 0.982 | 0.079 | 3.30 | 0.0E+00 | 0.0E+00 |
| Fabp4 | 25 | 0.937 | 0.006 | 2.63 | 0.0E+00 | 0.0E+00 |
| Plvap | 25 | 0.878 | 0.01 | 2.31 | 0.0E+00 | 0.0E+00 |
| Tm4sf1 | 25 | 0.869 | 0.003 | 1.92 | 0.0E+00 | 0.0E+00 |
| Ramp2 | 25 | 0.819 | 0.004 | 1.53 | 0.0E+00 | 0.0E+00 |
| Egfl7 | 25 | 0.837 | 0.004 | 1.30 | 0.0E+00 | 0.0E+00 |
| Esam | 25 | 0.801 | 0.007 | 1.27 | 0.0E+00 | 0.0E+00 |
| Cav1 | 25 | 0.729 | 0.003 | 1.15 | 0.0E+00 | 0.0E+00 |
| Flt1 | 25 | 0.783 | 0.002 | 1.11 | 0.0E+00 | 0.0E+00 |
| Emcn | 25 | 0.692 | 0.001 | 1.02 | 0.0E+00 | 0.0E+00 |
| Crip2 | 25 | 0.792 | 0.019 | 1.22 | 6.3E−311 | 1.2E−306 |
| Apold1 | 25 | 0.715 | 0.004 | 1.47 | 0.0E+00 | 1.1E−304 |
| Ifitm3 | 25 | 0.95 | 0.059 | 2.03 | 2.1E−299 | 4.0E−295 |
| Mgll | 25 | 0.706 | 0.021 | 1.07 | 1.9E−296 | 3.6E−292 |
| Ecscr | 25 | 0.674 | 0.004 | 0.97 | 5.8E−291 | 1.1E−286 |
| Tmem252 | 25 | 0.584 | 0.001 | 1.19 | 4.8E−282 | 9.2E−278 |
| Ptprb | 25 | 0.597 | 0.002 | 0.82 | 1.5E−279 | 2.8E−275 |
| Mcpt1 | 26 | 0.899 | 0.071 | 6.05 | 0.0E+00 | 0.0E+00 |
| Mcpt2 | 26 | 0.894 | 0.041 | 5.51 | 0.0E+00 | 0.0E+00 |
| Ifitm1 | 26 | 0.991 | 0.034 | 3.82 | 0.0E+00 | 0.0E+00 |
| Cpa3 | 26 | 0.991 | 0.011 | 3.73 | 0.0E+00 | 0.0E+00 |
| Cd63 | 26 | 0.991 | 0.053 | 3.36 | 0.0E+00 | 0.0E+00 |
| Fcer1a | 26 | 0.982 | 0.008 | 3.13 | 0.0E+00 | 0.0E+00 |
| Mcpt4 | 26 | 0.77 | 0.003 | 3.06 | 0.0E+00 | 0.0E+00 |
| Ifitm2 | 26 | 0.986 | 0.085 | 2.80 | 0.0E+00 | 0.0E+00 |
| Ier3 | 26 | 0.862 | 0.035 | 2.58 | 0.0E+00 | 0.0E+00 |
| Prnp | 26 | 0.899 | 0.028 | 2.47 | 0.0E+00 | 0.0E+00 |
| Tph1 | 26 | 0.889 | 0.005 | 2.46 | 0.0E+00 | 0.0E+00 |
| Furin | 26 | 0.834 | 0.161 | 1.99 | 0.0E+00 | 0.0E+00 |
| Scin | 26 | 0.899 | 0.005 | 1.97 | 0.0E+00 | 0.0E+00 |
| Srgn | 26 | 1 | 0.784 | 1.94 | 0.0E+00 | 0.0E+00 |
| Cyp11a1 | 26 | 0.917 | 0.004 | 1.90 | 0.0E+00 | 0.0E+00 |
| Ms4a2 | 26 | 0.889 | 0.002 | 1.86 | 0.0E+00 | 0.0E+00 |
| Gata2 | 26 | 0.866 | 0.004 | 1.76 | 0.0E+00 | 0.0E+00 |
| Cmtm7 | 26 | 0.972 | 0.323 | 1.53 | 0.0E+00 | 0.0E+00 |
| Cd34 | 26 | 0.876 | 0.008 | 1.44 | 0.0E+00 | 0.0E+00 |
| Gmpr | 26 | 0.857 | 0.005 | 1.36 | 0.0E+00 | 0.0E+00 |
| Itga2b | 26 | 0.719 | 0.003 | 1.10 | 0.0E+00 | 0.0E+00 |
| Car8 | 26 | 0.677 | 0.002 | 0.95 | 1.5E−301 | 2.9E−297 |
| Npl | 26 | 0.742 | 0.02 | 1.14 | 3.3E−300 | 6.3E−296 |
| Cx3cr1 | 26 | 0.783 | 0.009 | 1.11 | 3.8E−283 | 7.2E−279 |
| Mcpt9 | 26 | 0.59 | 0.002 | 2.54 | 1.2E−275 | 2.4E−271 |
| Gpx4 | 26 | 0.968 | 0.673 | 1.37 | 2.7E−274 | 5.3E−270 |
| Il1rl1 | 26 | 0.724 | 0.007 | 0.96 | 7.9E−274 | 1.5E−269 |
| Fabp2 | 30 | 0.963 | 0.058 | 5.36 | 0.0E+00 | 0.0E+00 |
| Fabp1 | 30 | 0.904 | 0.029 | 4.81 | 0.0E+00 | 0.0E+00 |
| Rbp2 | 30 | 0.926 | 0.017 | 4.50 | 0.0E+00 | 0.0E+00 |
| Aldob | 30 | 0.956 | 0.014 | 3.33 | 0.0E+00 | 0.0E+00 |
| Crip1 | 30 | 0.978 | 0.596 | 2.94 | 0.0E+00 | 0.0E+00 |
| Lgals4 | 30 | 0.993 | 0.026 | 2.92 | 0.0E+00 | 0.0E+00 |
| 2200002D01Rik | 30 | 0.933 | 0.029 | 2.77 | 0.0E+00 | 0.0E+00 |
| Dstn | 30 | 0.956 | 0.14 | 2.44 | 0.0E+00 | 0.0E+00 |
| Cystm1 | 30 | 0.919 | 0.028 | 2.39 | 0.0E+00 | 0.0E+00 |
| Fth1 | 30 | 0.993 | 0.98 | 2.31 | 0.0E+00 | 0.0E+00 |
| Txn1 | 30 | 0.97 | 0.522 | 2.24 | 0.0E+00 | 0.0E+00 |
| Scp2 | 30 | 0.933 | 0.593 | 2.18 | 0.0E+00 | 0.0E+00 |
| Smim24 | 30 | 0.83 | 0.069 | 2.15 | 0.0E+00 | 0.0E+00 |
| Dbi | 30 | 0.941 | 0.329 | 2.08 | 0.0E+00 | 0.0E+00 |
| Mdh1 | 30 | 0.919 | 0.505 | 1.89 | 0.0E+00 | 0.0E+00 |
| Atpif1 | 30 | 0.919 | 0.359 | 1.86 | 0.0E+00 | 0.0E+00 |
| Prdx1 | 30 | 0.941 | 0.662 | 1.79 | 0.0E+00 | 0.0E+00 |
| Hadh | 30 | 0.859 | 0.137 | 1.79 | 0.0E+00 | 0.0E+00 |
| Cyb5r3 | 30 | 0.778 | 0.178 | 1.74 | 0.0E+00 | 0.0E+00 |
| Cyb5a | 30 | 0.874 | 0.382 | 1.70 | 0.0E+00 | 0.0E+00 |
| Cbr1 | 30 | 0.77 | 0.109 | 1.65 | 0.0E+00 | 0.0E+00 |
| Cycs | 30 | 0.889 | 0.505 | 1.65 | 0.0E+00 | 0.0E+00 |
| Slc9a3r1 | 30 | 0.867 | 0.407 | 1.57 | 0.0E+00 | 0.0E+00 |
| Glrx | 30 | 0.8 | 0.123 | 1.55 | 0.0E+00 | 0.0E+00 |
| Atp5o | 30 | 0.859 | 0.559 | 1.46 | 0.0E+00 | 0.0E+00 |
| Rfk | 30 | 0.77 | 0.126 | 1.43 | 0.0E+00 | 0.0E+00 |
| Txndc17 | 30 | 0.83 | 0.327 | 1.39 | 0.0E+00 | 0.0E+00 |
| Pepd | 30 | 0.711 | 0.11 | 1.24 | 0.0E+00 | 0.0E+00 |
| Edf1 | 30 | 0.926 | 0.649 | 1.38 | 1.8E−310 | 3.6E−306 |
| Phgr1 | 30 | 0.926 | 0.008 | 2.86 | 0.0E+00 | 2.9E−305 |
| Acaa1a | 30 | 0.733 | 0.153 | 1.17 | 0.0E+00 | 9.2E−305 |
| Cndp2 | 30 | 0.659 | 0.125 | 1.16 | 1.2E−307 | 2.3E−303 |
| Mdh2 | 30 | 0.889 | 0.522 | 1.78 | 4.5E−305 | 8.6E−301 |
| Cox5a | 30 | 0.933 | 0.594 | 1.41 | 8.4E−297 | 1.6E−292 |
| Slc25a5 | 30 | 0.941 | 0.713 | 1.60 | 4.3E−293 | 8.3E−289 |
| Atp5j2 | 30 | 0.933 | 0.746 | 1.31 | 4.8E−290 | 9.2E−286 |
| Uqcrq | 30 | 0.911 | 0.569 | 1.36 | 8.3E−288 | 1.6E−283 |
| Abhd11os | 30 | 0.852 | 0.003 | 1.59 | 1.5E−281 | 2.9E−277 |
| Krt20 | 30 | 0.844 | 0.004 | 2.03 | 5.4E−280 | 1.0E−275 |
| Ckmt1 | 30 | 0.837 | 0.005 | 2.24 | 4.6E−279 | 8.8E−275 |
| Sephs2 | 30 | 0.741 | 0.16 | 1.17 | 5.4E−277 | 1.0E−272 |
| Prap1 | 30 | 0.852 | 0.005 | 2.71 | 6.4E−276 | 1.2E−271 |
| Calml4 | 30 | 0.852 | 0.005 | 1.72 | 8.0E−275 | 1.5E−270 |
| Uqcrb | 30 | 0.889 | 0.585 | 1.33 | 8.6E−274 | 1.7E−269 |
| Cst3 | 31 | 0.978 | 0.583 | 2.21 | 0.0E+00 | 0.0E+00 |
| Epsti1 | 31 | 0.425 | 0.174 | 1.60 | 0.0E+00 | 0.0E+00 |
| Ccl21a | 33 | 0.695 | 0.036 | 5.74 | 0.0E+00 | 0.0E+00 |
| Mfge8 | 33 | 0.952 | 0.048 | 3.74 | 0.0E+00 | 0.0E+00 |
| Igfbp7 | 33 | 0.981 | 0.082 | 3.64 | 1.8E−272 | 3.4E−268 |
| Oat | 35 | 0.717 | 0.164 | 2.24 | 0.0E+00 | 0.0E+00 |
| Spint2 | 35 | 0.869 | 0.365 | 2.15 | 8.8E−311 | 1.7E−306 |
| Msrb1 | 36 | 0.989 | 0.208 | 2.03 | 0.0E+00 | 0.0E+00 |
| Mif | 37 | 1 | 0.593 | 2.36 | 1.2E−276 | 2.3E−272 |
| Defa24 | 39 | 0.328 | 0.068 | 7.77 | 0.0E+00 | 0.0E+00 |
| Itln1 | 39 | 0.344 | 0.027 | 5.40 | 0.0E+00 | 0.0E+00 |

TABLE 2
Differential expression analysis between pairs of clusters in PP regions, related to FIG. 1. In certain
embodiments, the genes can be used as markers for the cell types represented by each cluster. The table
disclosed here represents only the genes with adjusted p values of 0 or less than or equal to 1.0E−150
and the genes were expressed in greater than 50% of cell type 1 and less than 10% of cell type 2.

| | cluster ID | cluster ID | percentage of | percentage of | | | |
|---|---|---|---|---|---|---|---|
| | of cell | of cell | expressing cells | expressing cells | LN (average | | p value, |
| gene | type 1 | type 2 | in cell type 1 | in cell type 2 | fold change) | p value | adjusted |
| Ms4a4b | 1 | 2 | 0.884 | 0.074 | 1.28E+00 | 0.00E+00 | 0.00E+00 |
| Trbc1 | 1 | 2 | 0.612 | 0.061 | 1.26E+00 | 0.00E+00 | 0.00E+00 |
| Cd3g | 1 | 2 | 0.907 | 0.052 | 1.26E+00 | 0.00E+00 | 0.00E+00 |
| Igfbp4 | 1 | 2 | 0.632 | 0.03 | 1.23E+00 | 0.00E+00 | 0.00E+00 |
| Cd3d | 1 | 2 | 0.892 | 0.073 | 1.19E+00 | 0.00E+00 | 0.00E+00 |
| Trac | 1 | 2 | 0.803 | 0.034 | 1.09E+00 | 0.00E+00 | 0.00E+00 |
| Cd3e | 1 | 2 | 0.846 | 0.057 | 1.09E+00 | 0.00E+00 | 0.00E+00 |
| Lef1 | 1 | 2 | 0.804 | 0.046 | 1.09E+00 | 0.00E+00 | 0.00E+00 |
| Lat | 1 | 2 | 0.755 | 0.055 | 8.95E−01 | 0.00E+00 | 0.00E+00 |
| Skap1 | 1 | 2 | 0.674 | 0.038 | 7.44E−01 | 0.00E+00 | 0.00E+00 |
| Fyb | 1 | 2 | 0.646 | 0.05 | 7.11E−01 | 0.00E+00 | 0.00E+00 |
| Thy1 | 1 | 2 | 0.546 | 0.033 | 6.87E−01 | 0.00E+00 | 0.00E+00 |
| Tcf7 | 1 | 2 | 0.531 | 0.026 | 5.85E−01 | 0.00E+00 | 0.00E+00 |
| Txk | 1 | 2 | 0.509 | 0.032 | 5.65E−01 | 0.00E+00 | 0.00E+00 |
| Igfbp4 | 1 | 3 | 0.632 | 0.022 | 1.23E+00 | 0.00E+00 | 0.00E+00 |
| Ms4a4b | 1 | 3 | 0.884 | 0.088 | 1.22E+00 | 0.00E+00 | 0.00E+00 |
| Cd3d | 1 | 3 | 0.892 | 0.031 | 1.22E+00 | 0.00E+00 | 0.00E+00 |
| Cd3g | 1 | 3 | 0.907 | 0.081 | 1.12E+00 | 0.00E+00 | 0.00E+00 |
| Trac | 1 | 3 | 0.803 | 0.062 | 1.06E+00 | 0.00E+00 | 0.00E+00 |
| Cd3e | 1 | 3 | 0.846 | 0.047 | 1.01E+00 | 0.00E+00 | 0.00E+00 |
| Ccr7 | 1 | 3 | 0.617 | 0.03 | 8.61E−01 | 0.00E+00 | 0.00E+00 |
| Ifi27l2a | 1 | 3 | 0.578 | 0.043 | 8.38E−01 | 0.00E+00 | 0.00E+00 |
| Cd2 | 1 | 3 | 0.651 | 0.023 | 7.46E−01 | 0.00E+00 | 0.00E+00 |
| Cd27 | 1 | 3 | 0.632 | 0.097 | 6.41E−01 | 0.00E+00 | 0.00E+00 |
| Emp3 | 1 | 3 | 0.56 | 0.076 | 5.74E−01 | 0.00E+00 | 0.00E+00 |
| Sell | 1 | 3 | 0.548 | 0.064 | 5.65E−01 | 0.00E+00 | 0.00E+00 |
| Ms4a4b | 1 | 4 | 0.884 | 0.027 | 1.33E+00 | 0.00E+00 | 0.00E+00 |
| Cd3g | 1 | 4 | 0.907 | 0.032 | 1.25E+00 | 0.00E+00 | 0.00E+00 |
| Cd3d | 1 | 4 | 0.892 | 0.016 | 1.24E+00 | 0.00E+00 | 0.00E+00 |
| Igfbp4 | 1 | 4 | 0.632 | 0.09 | 1.14E+00 | 0.00E+00 | 0.00E+00 |
| Cd3e | 1 | 4 | 0.846 | 0.012 | 1.12E+00 | 0.00E+00 | 0.00E+00 |
| Trac | 1 | 4 | 0.803 | 0.012 | 1.11E+00 | 0.00E+00 | 0.00E+00 |
| Lef1 | 1 | 4 | 0.804 | 0.029 | 1.10E+00 | 0.00E+00 | 0.00E+00 |
| Ms4a6b | 1 | 4 | 0.745 | 0.027 | 9.17E−01 | 0.00E+00 | 0.00E+00 |
| Ifi27l2a | 1 | 4 | 0.578 | 0.038 | 8.50E−01 | 0.00E+00 | 0.00E+00 |
| Ccr7 | 1 | 4 | 0.617 | 0.066 | 8.01E−01 | 0.00E+00 | 0.00E+00 |
| Cd2 | 1 | 4 | 0.651 | 0.011 | 7.59E−01 | 0.00E+00 | 0.00E+00 |
| Cd27 | 1 | 4 | 0.632 | 0.087 | 6.50E−01 | 0.00E+00 | 0.00E+00 |
| Sell | 1 | 4 | 0.548 | 0.064 | 5.66E−01 | 0.00E+00 | 0.00E+00 |
| Trbc2 | 1 | 6 | 0.962 | 0.084 | 1.76E+00 | 0.00E+00 | 0.00E+00 |
| Ms4a4b | 1 | 6 | 0.884 | 0.057 | 1.29E+00 | 0.00E+00 | 0.00E+00 |
| Cd3g | 1 | 6 | 0.907 | 0.024 | 1.28E+00 | 0.00E+00 | 0.00E+00 |
| Igfbp4 | 1 | 6 | 0.632 | 0.014 | 1.24E+00 | 0.00E+00 | 0.00E+00 |
| Cd3d | 1 | 6 | 0.892 | 0.026 | 1.23E+00 | 0.00E+00 | 0.00E+00 |
| Lef1 | 1 | 6 | 0.804 | 0.011 | 1.12E+00 | 0.00E+00 | 0.00E+00 |
| Cd3e | 1 | 6 | 0.846 | 0.02 | 1.12E+00 | 0.00E+00 | 0.00E+00 |
| Trac | 1 | 6 | 0.803 | 0.013 | 1.11E+00 | 0.00E+00 | 0.00E+00 |
| Lat | 1 | 6 | 0.755 | 0.098 | 8.34E−01 | 0.00E+00 | 0.00E+00 |
| Lck | 1 | 6 | 0.762 | 0.098 | 8.33E−01 | 0.00E+00 | 0.00E+00 |
| Skap1 | 1 | 6 | 0.674 | 0.033 | 7.48E−01 | 0.00E+00 | 0.00E+00 |
| Cd2 | 1 | 6 | 0.651 | 0.031 | 7.37E−01 | 0.00E+00 | 0.00E+00 |
| Thy1 | 1 | 6 | 0.546 | 0.092 | 6.14E−01 | 0.00E+00 | 0.00E+00 |
| Tcf7 | 1 | 6 | 0.531 | 0.058 | 5.47E−01 | 3.02E−285 | 5.35E−278 |
| Trbc1 | 1 | 6 | 0.612 | 0.07 | 1.23E+00 | 1.52E−280 | 2.68E−273 |
| Txk | 1 | 6 | 0.509 | 0.076 | 5.15E−01 | 5.04E−207 | 8.93E−200 |
| Igfbp4 | 1 | 7 | 0.632 | 0.058 | 1.15E+00 | 0.00E+00 | 0.00E+00 |
| Igfbp4 | 1 | 8 | 0.632 | 0.013 | 1.23E+00 | 0.00E+00 | 0.00E+00 |
| Cd3e | 1 | 8 | 0.846 | 0.016 | 1.12E+00 | 0.00E+00 | 0.00E+00 |
| Trac | 1 | 8 | 0.803 | 0.056 | 1.06E+00 | 0.00E+00 | 0.00E+00 |
| Ifi27l2a | 1 | 8 | 0.578 | 0.054 | 8.03E−01 | 0.00E+00 | 0.00E+00 |
| Rgs10 | 1 | 8 | 0.583 | 0.099 | 6.06E−01 | 0.00E+00 | 0.00E+00 |
| Bcl2 | 1 | 9 | 0.744 | 0.095 | 1.20E+00 | 0.00E+00 | 0.00E+00 |
| Igfbp4 | 1 | 9 | 0.632 | 0.066 | 1.19E+00 | 0.00E+00 | 0.00E+00 |
| Lef1 | 1 | 9 | 0.804 | 0.057 | 1.07E+00 | 0.00E+00 | 0.00E+00 |
| Trac | 1 | 9 | 0.803 | 0.083 | 1.04E+00 | 0.00E+00 | 0.00E+00 |
| Skap1 | 1 | 9 | 0.674 | 0.088 | 6.92E−01 | 0.00E+00 | 0.00E+00 |
| Thy1 | 1 | 9 | 0.546 | 0.086 | 6.26E−01 | 4.2E−310 | 7.37E−303 |
| Satb1 | 1 | 9 | 0.622 | 0.069 | 6.80E−01 | 1.35E−297 | 2.40E−290 |
| Itgb7 | 1 | 9 | 0.545 | 0.075 | 5.40E−01 | 2.71E−259 | 4.80E−252 |
| Tcf7 | 1 | 9 | 0.531 | 0.052 | 5.52E−01 | 6.98E−216 | 1.24E−208 |
| Sell | 1 | 9 | 0.548 | 0.062 | 5.69E−01 | 1.72E−207 | 3.05E−200 |
| S100a10 | 1 | 9 | 0.545 | 0.093 | 4.84E−01 | 5.57E−181 | 9.87E−174 |
| Ms4a4b | 1 | 10 | 0.884 | 0.07 | 1.28E+00 | 0.00E+00 | 0.00E+00 |
| Trbcl | 1 | 10 | 0.612 | 0.064 | 1.26E+00 | 0.00E+00 | 0.00E+00 |
| Bcl2 | 1 | 10 | 0.744 | 0.05 | 1.26E+00 | 0.00E+00 | 0.00E+00 |
| Cd3g | 1 | 10 | 0.907 | 0.055 | 1.25E+00 | 0.00E+00 | 0.00E+00 |
| Igfbp4 | 1 | 10 | 0.632 | 0.037 | 1.23E+00 | 0.00E+00 | 0.00E+00 |
| Cd3d | 1 | 10 | 0.892 | 0.083 | 1.18E+00 | 0.00E+00 | 0.00E+00 |
| Lef1 | 1 | 10 | 0.804 | 0.031 | 1.10E+00 | 0.00E+00 | 0.00E+00 |
| Trac | 1 | 10 | 0.803 | 0.05 | 1.08E+00 | 0.00E+00 | 0.00E+00 |
| Cd3e | 1 | 10 | 0.846 | 0.07 | 1.07E+00 | 0.00E+00 | 0.00E+00 |
| Lat | 1 | 10 | 0.755 | 0.076 | 8.77E−01 | 0.00E+00 | 0.00E+00 |
| Ccnd2 | 1 | 10 | 0.669 | 0.044 | 8.10E−01 | 0.00E+00 | 0.00E+00 |
| Ccr7 | 1 | 10 | 0.617 | 0.069 | 8.07E−01 | 0.00E+00 | 0.00E+00 |
| Selplg | 1 | 10 | 0.677 | 0.09 | 7.56E−01 | 0.00E+00 | 0.00E+00 |
| Skap1 | 1 | 10 | 0.674 | 0.035 | 7.47E−01 | 0.00E+00 | 0.00E+00 |
| Hcst | 1 | 10 | 0.662 | 0.089 | 7.41E−01 | 0.00E+00 | 0.00E+00 |
| Fyb | 1 | 10 | 0.646 | 0.059 | 7.02E−01 | 0.00E+00 | 0.00E+00 |
| Satb1 | 1 | 10 | 0.622 | 0.061 | 6.88E−01 | 0.00E+00 | 0.00E+00 |
| Cd2 | 1 | 10 | 0.651 | 0.074 | 6.77E−01 | 0.00E+00 | 0.00E+00 |
| Thy1 | 1 | 10 | 0.546 | 0.049 | 6.71E−01 | 0.00E+00 | 0.00E+00 |
| Sell | 1 | 10 | 0.548 | 0.042 | 5.90E−01 | 0.00E+00 | 0.00E+00 |
| Tcf7 | 1 | 10 | 0.531 | 0.029 | 5.83E−01 | 0.00E+00 | 0.00E+00 |
| Emp3 | 1 | 10 | 0.56 | 0.071 | 5.80E−01 | 0.00E+00 | 0.00E+00 |
| Itgb7 | 1 | 10 | 0.545 | 0.058 | 5.68E−01 | 0.00E+00 | 0.00E+00 |
| Txk | 1 | 10 | 0.509 | 0.03 | 5.69E−01 | 2.54E−292 | 4.50E−285 |
| Igfbp4 | 1 | 11 | 0.632 | 0.009 | 1.25E+00 | 0.00E+00 | 0.00E+00 |
| Cd3d | 1 | 11 | 0.892 | 0.047 | 1.21E+00 | 0.00E+00 | 0.00E+00 |
| Cd3g | 1 | 11 | 0.907 | 0.069 | 1.17E+00 | 0.00E+00 | 0.00E+00 |
| Cd3e | 1 | 11 | 0.846 | 0.021 | 1.11E+00 | 0.00E+00 | 0.00E+00 |
| Lef1 | 1 | 11 | 0.804 | 0.058 | 1.07E+00 | 0.00E+00 | 0.00E+00 |
| Ccr7 | 1 | 11 | 0.617 | 0.035 | 8.56E−01 | 0.00E+00 | 0.00E+00 |
| Ifi27l2a | 1 | 11 | 0.578 | 0.07 | 8.00E−01 | 7.21E−255 | 1.28E−247 |
| Sell | 1 | 11 | 0.548 | 0.094 | 5.24E−01 | 8.98E−225 | 1.59E−217 |
| Ms4a4b | 1 | 12 | 0.884 | 0.051 | 1.29E+00 | 0.00E+00 | 0.00E+00 |
| Cd3d | 1 | 12 | 0.892 | 0.028 | 1.23E+00 | 0.00E+00 | 0.00E+00 |
| Igfbp4 | 1 | 12 | 0.632 | 0.03 | 1.20E+00 | 0.00E+00 | 0.00E+00 |
| Trac | 1 | 12 | 0.803 | 0.014 | 1.12E+00 | 0.00E+00 | 0.00E+00 |
| Cd3e | 1 | 12 | 0.846 | 0.057 | 1.07E+00 | 0.00E+00 | 0.00E+00 |
| Lef1 | 1 | 12 | 0.804 | 0.073 | 1.06E+00 | 0.00E+00 | 0.00E+00 |
| Ms4a6b | 1 | 12 | 0.745 | 0.08 | 8.58E−01 | 0.00E+00 | 0.00E+00 |
| Ccr7 | 1 | 12 | 0.617 | 0.047 | 8.25E−01 | 0.00E+00 | 0.00E+00 |
| Cd2 | 1 | 12 | 0.651 | 0.026 | 7.43E−01 | 0.00E+00 | 0.00E+00 |
| Ifi27l2a | 1 | 12 | 0.578 | 0.05 | 8.36E−01 | 4.79E−257 | 8.48E−250 |
| Sell | 1 | 12 | 0.548 | 0.081 | 5.45E−01 | 3.43E−226 | 6.07E−219 |
| Cd3d | 1 | 14 | 0.892 | 0.061 | 1.16E+00 | 0.00E+00 | 0.00E+00 |
| Igfbp4 | 1 | 14 | 0.632 | 0.066 | 1.13E+00 | 0.00E+00 | 0.00E+00 |
| Cd3e | 1 | 14 | 0.846 | 0.038 | 1.07E+00 | 0.00E+00 | 0.00E+00 |
| Lat | 1 | 14 | 0.755 | 0.053 | 8.72E−01 | 0.00E+00 | 0.00E+00 |
| Trac | 1 | 14 | 0.803 | 0.033 | 1.07E+00 | 3.82E−308 | 6.76E−301 |
| Cd3g | 1 | 14 | 0.907 | 0.058 | 1.19E+00 | 7.15E−303 | 1.27E−295 |
| Skap1 | 1 | 14 | 0.674 | 0.072 | 6.92E−01 | 4.35E−271 | 7.69E−264 |
| Cd2 | 1 | 14 | 0.651 | 0.058 | 6.94E−01 | 6.52E−249 | 1.16E−241 |
| Thy1 | 1 | 14 | 0.546 | 0.085 | 6.21E−01 | 2.23E−247 | 3.95E−240 |
| Cd3d | 1 | 15 | 0.892 | 0.013 | 1.25E+00 | 5.73E−259 | 1.01E−251 |
| Cd3g | 1 | 15 | 0.907 | 0.04 | 1.26E+00 | 9.38E−259 | 1.66E−251 |
| Ms4a4b | 1 | 15 | 0.884 | 0.032 | 1.32E+00 | 4.71E−243 | 8.34E−236 |
| Cd3e | 1 | 15 | 0.846 | 0.025 | 1.12E+00 | 1.87E−198 | 3.31E−191 |
| Trac | 1 | 15 | 0.803 | 0.013 | 1.11E+00 | 1.47E−192 | 2.61E−185 |
| Lef1 | 1 | 15 | 0.804 | 0.034 | 1.10E+00 | 3.09E−170 | 5.47E−163 |
| Igfbp4 | 1 | 17 | 0.632 | 0.023 | 1.19E+00 | 1.92E−274 | 3.40E−267 |
| Lef1 | 1 | 17 | 0.804 | 0.062 | 1.05E+00 | 3.70E−268 | 6.54E−261 |
| Ccr7 | 1 | 17 | 0.617 | 0.062 | 8.27E−01 | 1.49E−178 | 2.63E−171 |
| Cd2 | 1 | 17 | 0.651 | 0.082 | 6.70E−01 | 4.20E−168 | 7.43E−161 |
| Igfbp4 | 1 | 20 | 0.632 | 0.042 | 1.20E+00 | 2.40E−263 | 4.25E−256 |
| Cd3e | 1 | 20 | 0.846 | 0.064 | 1.00E+00 | 5.78E−250 | 1.02E−242 |
| Cd3d | 1 | 20 | 0.892 | 0.093 | 1.09E+00 | 9.39E−239 | 1.66E−231 |
| Cd3g | 1 | 20 | 0.907 | 0.081 | 1.17E+00 | 7.45E−235 | 1.32E−227 |
| Lef1 | 1 | 20 | 0.804 | 0.034 | 1.10E+00 | 1.92E−233 | 3.40E−226 |
| Trac | 1 | 20 | 0.803 | 0.064 | 1.05E+00 | 1.52E−211 | 2.68E−204 |
| Skap1 | 1 | 20 | 0.674 | 0.047 | 7.29E−01 | 3.49E−167 | 6.17E−160 |
| Igfbp4 | 1 | 21 | 0.632 | 0.073 | 1.13E+00 | 0.00E+00 | 0.00E+00 |
| Cd3e | 1 | 21 | 0.846 | 0.064 | 1.04E+00 | 1.92E−234 | 3.39E−227 |
| Lat | 1 | 21 | 0.755 | 0.098 | 8.26E−01 | 5.52E−234 | 9.77E−227 |
| Lef1 | 1 | 21 | 0.804 | 0.038 | 1.05E+00 | 5.55E−206 | 9.82E−199 |
| Cd3d | 1 | 21 | 0.892 | 0.064 | 1.14E+00 | 4.12E−196 | 7.29E−189 |
| Trac | 1 | 21 | 0.803 | 0.034 | 1.06E+00 | 4.30E−184 | 7.61E−177 |
| Skap1 | 1 | 21 | 0.674 | 0.073 | 6.97E−01 | 2.20E−169 | 3.89E−162 |
| Cd3g | 1 | 21 | 0.907 | 0.098 | 1.11E+00 | 8.39E−168 | 1.49E−160 |
| Cd2 | 1 | 21 | 0.651 | 0.06 | 7.07E−01 | 1.01E−167 | 1.78E−160 |
| Igfbp4 | 1 | 22 | 0.632 | 0.065 | 1.19E+00 | 2.64E−257 | 4.68E−250 |
| Cd3d | 1 | 22 | 0.892 | 0.022 | 1.24E+00 | 5.10E−251 | 9.02E−244 |
| Cd3e | 1 | 22 | 0.846 | 0.039 | 1.10E+00 | 1.11E−249 | 1.97E−242 |
| Lef1 | 1 | 22 | 0.804 | 0.034 | 1.10E+00 | 8.02E−233 | 1.42E−225 |
| Cd3g | 1 | 22 | 0.907 | 0.039 | 1.27E+00 | 9.46E−231 | 1.68E−223 |
| Lat | 1 | 22 | 0.755 | 0.047 | 9.04E−01 | 3.23E−219 | 5.72E−212 |
| Trac | 1 | 22 | 0.803 | 0.013 | 1.12E+00 | 3.49E−218 | 6.17E−211 |
| Lck | 1 | 22 | 0.762 | 0.082 | 8.19E−01 | 4.80E−202 | 8.51E−195 |
| Skap1 | 1 | 22 | 0.674 | 0.047 | 7.37E−01 | 3.47E−169 | 6.14E−162 |
| Igfbp4 | 1 | 24 | 0.632 | 0.031 | 1.23E+00 | 4.23E−268 | 7.50E−261 |
| Cd3e | 1 | 24 | 0.846 | 0.004 | 1.14E+00 | 6.11E−261 | 1.08E−253 |
| Cd3d | 1 | 24 | 0.892 | 0.018 | 1.25E+00 | 5.34E−244 | 9.46E−237 |
| Cd3g | 1 | 24 | 0.907 | 0.027 | 1.22E+00 | 4.55E−231 | 8.06E−224 |
| Trbc2 | 1 | 24 | 0.962 | 0.081 | 1.73E+00 | 8.05E−228 | 1.43E−220 |
| Bcl2 | 1 | 24 | 0.744 | 0.094 | 1.11E+00 | 8.83E−227 | 1.56E−219 |
| Ms4a4b | 1 | 24 | 0.884 | 0.031 | 1.31E+00 | 1.08E−223 | 1.91E−216 |
| Lat | 1 | 24 | 0.755 | 0.027 | 9.28E−01 | 6.40E−222 | 1.13E−214 |
| Lef1 | 1 | 24 | 0.804 | 0.04 | 1.07E+00 | 2.05E−221 | 3.63E−214 |
| Trac | 1 | 24 | 0.803 | 0.013 | 1.11E+00 | 4.43E−212 | 7.84E−205 |
| Lck | 1 | 24 | 0.762 | 0.058 | 8.80E−01 | 1.94E−209 | 3.43E−202 |
| Gimap4 | 1 | 24 | 0.684 | 0.058 | 7.56E−01 | 1.39E−171 | 2.46E−164 |
| Skap1 | 1 | 24 | 0.674 | 0.031 | 7.43E−01 | 5.48E−164 | 9.70E−157 |
| Cd2 | 1 | 24 | 0.651 | 0.027 | 7.47E−01 | 4.77E−161 | 8.44E−154 |
| Coro1a | 1 | 25 | 0.969 | 0.1 | 1.56E+00 | 2.73E−307 | 4.83E−300 |
| Trbc2 | 1 | 25 | 0.962 | 0.063 | 1.78E+00 | 3.50E−290 | 6.20E−283 |
| Limd2 | 1 | 25 | 0.928 | 0.1 | 1.37E+00 | 3.75E−282 | 6.63E−275 |
| Cd3d | 1 | 25 | 0.892 | 0.018 | 1.25E+00 | 5.85E−242 | 1.04E−234 |
| Cd3g | 1 | 25 | 0.907 | 0.027 | 1.29E+00 | 3.89E−237 | 6.88E−230 |
| Cd3e | 1 | 25 | 0.846 | 0.009 | 1.13E+00 | 1.41E−226 | 2.49E−219 |
| Ms4a4b | 1 | 25 | 0.884 | 0.018 | 1.33E+00 | 1.38E−223 | 2.44E−216 |
| Lef1 | 1 | 25 | 0.804 | 0.014 | 1.12E+00 | 2.08E−194 | 3.68E−187 |
| Trac | 1 | 25 | 0.803 | 0.009 | 1.12E+00 | 1.07E−189 | 1.89E−182 |
| Lat | 1 | 25 | 0.755 | 0.018 | 9.36E−01 | 1.97E−170 | 3.48E−163 |
| Ptprcap | 1 | 25 | 0.766 | 0.063 | 9.42E−01 | 2.29E−169 | 4.05E−162 |
| Lck | 1 | 25 | 0.762 | 0.045 | 8.80E−01 | 1.18E−166 | 2.08E−159 |
| Cd3d | 1 | 26 | 0.892 | 0.032 | 1.23E+00 | 1.65E−246 | 2.93E−239 |
| Cd3e | 1 | 26 | 0.846 | 0.078 | 1.04E+00 | 1.45E−240 | 2.56E−233 |
| Cd3g | 1 | 26 | 0.907 | 0.014 | 1.28E+00 | 7.36E−237 | 1.30E−229 |
| Trbc2 | 1 | 26 | 0.962 | 0.083 | 1.73E+00 | 6.15E−236 | 1.09E−228 |
| Igfbp4 | 1 | 26 | 0.632 | 0.083 | 1.04E+00 | 1.95E−234 | 3.46E−227 |
| Ms4a4b | 1 | 26 | 0.884 | 0.06 | 1.27E+00 | 1.53E−227 | 2.71E−220 |
| Lef1 | 1 | 26 | 0.804 | 0.014 | 1.11E+00 | 2.28E−225 | 4.04E−218 |
| Trac | 1 | 26 | 0.803 | 0.005 | 1.12E+00 | 7.72E−214 | 1.37E−206 |
| Lck | 1 | 26 | 0.762 | 0.032 | 9.05E−01 | 9.29E−203 | 1.64E−195 |
| Ms4a6b | 1 | 26 | 0.745 | 0.028 | 8.94E−01 | 5.75E−166 | 1.02E−158 |
| Trbc2 | 1 | 30 | 0.962 | 0.059 | 1.78E+00 | 8.60E−194 | 1.52E−186 |
| Cd52 | 1 | 32 | 0.968 | 0.094 | 1.85E+00 | 1.35E−213 | 2.39E−206 |
| Coro1a | 1 | 32 | 0.969 | 0.085 | 1.59E+00 | 1.86E−190 | 3.29E−183 |
| Trbc2 | 1 | 32 | 0.962 | 0.017 | 1.86E+00 | 8.07E−175 | 1.43E−167 |
| Rac2 | 1 | 32 | 0.946 | 0.085 | 1.40E+00 | 2.49E−164 | 4.41E−157 |
| Cd52 | 1 | 33 | 0.968 | 0.076 | 1.91E+00 | 5.65E−191 | l.OOE−183 |
| Igfbp4 | 1 | 34 | 0.632 | 0.03 | 1.24E+00 | 2.01E−242 | 3.56E−235 |
| Cd3e | 1 | 34 | 0.846 | 0.04 | 1.11E+00 | 4.59E−179 | 8.13E−172 |
| Lat | 1 | 34 | 0.755 | 0.06 | 8.96E−01 | 1.61E−176 | 2.85E−169 |
| Cd3e | 1 | 35 | 0.846 | 0.051 | 1.08E+00 | 3.66E−180 | 6.47E−173 |
| Cd79a | 2 | 3 | 0.995 | 0.025 | 2.38E+00 | 0.00E+00 | 0.00E+00 |
| Iglc2 | 2 | 3 | 0.897 | 0.025 | 1.91E+00 | 0.00E+00 | 0.00E+00 |
| Apoe | 2 | 3 | 0.618 | 0.058 | 1.83E+00 | 0.00E+00 | 0.00E+00 |
| H2-DMb2 | 2 | 3 | 0.966 | 0.032 | 1.80E+00 | 0.00E+00 | 0.00E+00 |
| Ebf1 | 2 | 3 | 0.971 | 0.009 | 1.77E+00 | 0.00E+00 | 0.00E+00 |
| Iglc3 | 2 | 3 | 0.886 | 0.023 | 1.56E+00 | 0.00E+00 | 0.00E+00 |
| Iglc1 | 2 | 3 | 0.54 | 0.06 | 1.51E+00 | 0.00E+00 | 0.00E+00 |
| Ms4a1 | 2 | 3 | 0.876 | 0.01 | 1.38E+00 | 0.00E+00 | 0.00E+00 |
| Fcmr | 2 | 3 | 0.817 | 0.006 | 1.37E+00 | 0.00E+00 | 0.00E+00 |
| Mef2c | 2 | 3 | 0.811 | 0.025 | 1.20E+00 | 0.00E+00 | 0.00E+00 |
| Ifi30 | 2 | 3 | 0.787 | 0.087 | 1.18E+00 | 0.00E+00 | 0.00E+00 |
| Ighd | 2 | 3 | 0.738 | 0.005 | 1.17E+00 | 0.00E+00 | 0.00E+00 |
| Mzb1 | 2 | 3 | 0.671 | 0.032 | 1.01E+00 | 0.00E+00 | 0.00E+00 |
| Vpreb3 | 2 | 3 | 0.635 | 0.006 | 9.94E−01 | 0.00E+00 | 0.00E+00 |
| Tnfrsf13c | 2 | 3 | 0.687 | 0.008 | 9.26E−01 | 0.00E+00 | 0.00E+00 |
| Ccr7 | 2 | 3 | 0.544 | 0.03 | 8.89E−01 | 0.00E+00 | 0.00E+00 |
| Fcer2a | 2 | 3 | 0.613 | 0.004 | 8.78E−01 | 0.00E+00 | 0.00E+00 |
| Hvcn1 | 2 | 3 | 0.712 | 0.089 | 8.71E−01 | 0.00E+00 | 0.00E+00 |
| Cd24a | 2 | 3 | 0.59 | 0.011 | 8.58E−01 | 0.00E+00 | 0.00E+00 |
| Napsa | 2 | 3 | 0.686 | 0.071 | 8.44E−01 | 0.00E+00 | 0.00E+00 |
| Bank1 | 2 | 3 | 0.628 | 0.01 | 7.60E−01 | 0.00E+00 | 0.00E+00 |
| Pou2f2 | 2 | 3 | 0.578 | 0.026 | 7.26E−01 | 0.00E+00 | 0.00E+00 |
| Pkig | 2 | 3 | 0.611 | 0.057 | 7.05E−01 | 0.00E+00 | 0.00E+00 |
| Siglecg | 2 | 3 | 0.569 | 0.01 | 6.85E−01 | 0.00E+00 | 0.00E+00 |
| H2-Oa | 2 | 3 | 0.545 | 0.027 | 6.66E−01 | 0.00E+00 | 0.00E+00 |
| Fcrla | 2 | 3 | 0.535 | 0.022 | 6.62E−01 | 0.00E+00 | 0.00E+00 |
| Chchd10 | 2 | 3 | 0.549 | 0.09 | 6.54E−01 | 0.00E+00 | 0.00E+00 |
| H2-Ob | 2 | 3 | 0.542 | 0.006 | 6.30E−01 | 0.00E+00 | 0.00E+00 |
| Snn | 2 | 3 | 0.524 | 0.015 | 6.08E−01 | 0.00E+00 | 0.00E+00 |
| Cd79a | 2 | 4 | 0.995 | 0.027 | 2.36E+00 | 0.00E+00 | 0.00E+00 |
| Iglc2 | 2 | 4 | 0.897 | 0.024 | 1.90E+00 | 0.00E+00 | 0.00E+00 |
| Apoe | 2 | 4 | 0.618 | 0.057 | 1.84E+00 | 0.00E+00 | 0.00E+00 |
| H2-DMb2 | 2 | 4 | 0.966 | 0.07 | 1.70E+00 | 0.00E+00 | 0.00E+00 |
| Ebf1 | 2 | 4 | 0.971 | 0.062 | 1.67E+00 | 0.00E+00 | 0.00E+00 |
| Iglc3 | 2 | 4 | 0.886 | 0.029 | 1.55E+00 | 0.00E+00 | 0.00E+00 |
| Iglc1 | 2 | 4 | 0.54 | 0.071 | 1.50E+00 | 0.00E+00 | 0.00E+00 |
| Ms4a1 | 2 | 4 | 0.876 | 0.014 | 1.38E+00 | 0.00E+00 | 0.00E+00 |
| Fcmr | 2 | 4 | 0.817 | 0.007 | 1.37E+00 | 0.00E+00 | 0.00E+00 |
| Ifi30 | 2 | 4 | 0.787 | 0.071 | 1.20E+00 | 0.00E+00 | 0.00E+00 |
| Ighd | 2 | 4 | 0.738 | 0.009 | 1.16E+00 | 0.00E+00 | 0.00E+00 |
| Mzb1 | 2 | 4 | 0.671 | 0.033 | 1.00E+00 | 0.00E+00 | 0.00E+00 |
| Vpreb3 | 2 | 4 | 0.635 | 0.005 | 9.93E−01 | 0.00E+00 | 0.00E+00 |
| Dusp2 | 2 | 4 | 0.616 | 0.054 | 9.49E−01 | 0.00E+00 | 0.00E+00 |
| Tnfrsf13c | 2 | 4 | 0.687 | 0.007 | 9.28E−01 | 0.00E+00 | 0.00E+00 |
| Fcer2a | 2 | 4 | 0.613 | 0.006 | 8.73E−01 | 0.00E+00 | 0.00E+00 |
| Cd24a | 2 | 4 | 0.59 | 0.014 | 8.54E−01 | 0.00E+00 | 0.00E+00 |
| Ccr7 | 2 | 4 | 0.544 | 0.066 | 8.29E−01 | 0.00E+00 | 0.00E+00 |
| Bank1 | 2 | 4 | 0.628 | 0.008 | 7.60E−01 | 0.00E+00 | 0.00E+00 |
| Pou2f2 | 2 | 4 | 0.578 | 0.026 | 7.23E−01 | 0.00E+00 | 0.00E+00 |
| Chchd10 | 2 | 4 | 0.549 | 0.061 | 6.85E−01 | 0.00E+00 | 0.00E+00 |
| Fcrla | 2 | 4 | 0.535 | 0.015 | 6.69E−01 | 0.00E+00 | 0.00E+00 |
| Siglecg | 2 | 4 | 0.569 | 0.038 | 6.43E−01 | 0.00E+00 | 0.00E+00 |
| H2-Ob | 2 | 4 | 0.542 | 0.011 | 6.26E−01 | 0.00E+00 | 0.00E+00 |
| Snn | 2 | 4 | 0.524 | 0.022 | 6.01E−01 | 0.00E+00 | 0.00E+00 |
| Iglc2 | 2 | 5 | 0.897 | 0.071 | 1.86E+00 | 0.00E+00 | 0.00E+00 |
| Apoe | 2 | 5 | 0.618 | 0.085 | 1.78E+00 | 0.00E+00 | 0.00E+00 |
| H2-DMb2 | 2 | 5 | 0.966 | 0.068 | 1.76E+00 | 0.00E+00 | 0.00E+00 |
| Ebf1 | 2 | 5 | 0.971 | 0.032 | 1.75E+00 | 0.00E+00 | 0.00E+00 |
| Iglc3 | 2 | 5 | 0.886 | 0.045 | 1.54E+00 | 0.00E+00 | 0.00E+00 |
| Fcmr | 2 | 5 | 0.817 | 0.034 | 1.34E+00 | 0.00E+00 | 0.00E+00 |
| Ms4a1 | 2 | 5 | 0.876 | 0.05 | 1.34E+00 | 0.00E+00 | 0.00E+00 |
| Ifi30 | 2 | 5 | 0.787 | 0.059 | 1.21E+00 | 0.00E+00 | 0.00E+00 |
| Mef2c | 2 | 5 | 0.811 | 0.014 | 1.21E+00 | 0.00E+00 | 0.00E+00 |
| Cd83 | 2 | 5 | 0.735 | 0.037 | 1.19E+00 | 0.00E+00 | 0.00E+00 |
| Ighd | 2 | 5 | 0.738 | 0.015 | 1.16E+00 | 0.00E+00 | 0.00E+00 |
| Mzb1 | 2 | 5 | 0.671 | 0.048 | 9.91E−01 | 0.00E+00 | 0.00E+00 |
| Vpreb3 | 2 | 5 | 0.635 | 0.03 | 9.70E−01 | 0.00E+00 | 0.00E+00 |
| Napsa | 2 | 5 | 0.686 | 0.018 | 9.02E−01 | 0.00E+00 | 0.00E+00 |
| Tnfrsf13c | 2 | 5 | 0.687 | 0.034 | 9.01E−01 | 0.00E+00 | 0.00E+00 |
| Irf8 | 2 | 5 | 0.701 | 0.045 | 8.98E−01 | 0.00E+00 | 0.00E+00 |
| Fcer2a | 2 | 5 | 0.613 | 0.016 | 8.65E−01 | 0.00E+00 | 0.00E+00 |
| Cd24a | 2 | 5 | 0.59 | 0.043 | 8.26E−01 | 0.00E+00 | 0.00E+00 |
| Capg | 2 | 5 | 0.635 | 0.076 | 8.01E−01 | 0.00E+00 | 0.00E+00 |
| Bank1 | 2 | 5 | 0.628 | 0.02 | 7.50E−01 | 0.00E+00 | 0.00E+00 |
| Pkig | 2 | 5 | 0.611 | 0.02 | 7.43E−01 | 0.00E+00 | 0.00E+00 |
| Ctsh | 2 | 5 | 0.61 | 0.028 | 7.37E−01 | 0.00E+00 | 0.00E+00 |
| Unc93b1 | 2 | 5 | 0.593 | 0.029 | 6.96E−01 | 0.00E+00 | 0.00E+00 |
| Siglecg | 2 | 5 | 0.569 | 0.018 | 6.77E−01 | 0.00E+00 | 0.00E+00 |
| Fcrla | 2 | 5 | 0.535 | 0.016 | 6.69E−01 | 0.00E+00 | 0.00E+00 |
| Snn | 2 | 5 | 0.524 | 0.013 | 6.10E−01 | 0.00E+00 | 0.00E+00 |
| H2-Ob | 2 | 5 | 0.542 | 0.071 | 5.65E−01 | 0.00E+00 | 0.00E+00 |
| Cd79a | 2 | 6 | 0.995 | 0.056 | 2.29E+00 | 0.00E+00 | 0.00E+00 |
| Ebf1 | 2 | 6 | 0.971 | 0.023 | 1.73E+00 | 0.00E+00 | 0.00E+00 |
| Ms4a1 | 2 | 6 | 0.876 | 0.022 | 1.35E+00 | 0.00E+00 | 0.00E+00 |
| Fcmr | 2 | 6 | 0.817 | 0.019 | 1.35E+00 | 0.00E+00 | 0.00E+00 |
| Vpreb3 | 2 | 6 | 0.635 | 0.01 | 9.81E−01 | 0.00E+00 | 0.00E+00 |
| H2-Ob | 2 | 6 | 0.542 | 0.031 | 6.01E−01 | 5.5E−312 | 9.68E−305 |
| Ighd | 2 | 6 | 0.738 | 0.058 | 1.11E+00 | 6.49E−296 | 1.15E−288 |
| Fcer2a | 2 | 6 | 0.613 | 0.014 | 8.65E−01 | 8.28E−282 | 1.47E−274 |
| Dusp2 | 2 | 6 | 0.616 | 0.098 | 8.92E−01 | 3.32E−226 | 5.88E−219 |
| Iglc2 | 2 | 7 | 0.897 | 0.084 | 1.84E+00 | 0.00E+00 | 0.00E+00 |
| Ebf1 | 2 | 7 | 0.971 | 0.029 | 1.74E+00 | 0.00E+00 | 0.00E+00 |
| H2-DMb2 | 2 | 7 | 0.966 | 0.091 | 1.73E+00 | 0.00E+00 | 0.00E+00 |
| Iglc3 | 2 | 7 | 0.886 | 0.055 | 1.52E+00 | 0.00E+00 | 0.00E+00 |
| Iglc1 | 2 | 7 | 0.54 | 0.096 | 1.47E+00 | 0.00E+00 | 0.00E+00 |
| Fcmr | 2 | 7 | 0.817 | 0.039 | 1.34E+00 | 0.00E+00 | 0.00E+00 |
| Ms4a1 | 2 | 7 | 0.876 | 0.059 | 1.34E+00 | 0.00E+00 | 0.00E+00 |
| Mef2c | 2 | 7 | 0.811 | 0.019 | 1.21E+00 | 0.00E+00 | 0.00E+00 |
| Ifi30 | 2 | 7 | 0.787 | 0.072 | 1.20E+00 | 0.00E+00 | 0.00E+00 |
| Ighd | 2 | 7 | 0.738 | 0.033 | 1.14E+00 | 0.00E+00 | 0.00E+00 |
| Mzb1 | 2 | 7 | 0.671 | 0.056 | 9.76E−01 | 0.00E+00 | 0.00E+00 |
| Vpreb3 | 2 | 7 | 0.635 | 0.037 | 9.61E−01 | 0.00E+00 | 0.00E+00 |
| Tnfrsf13c | 2 | 7 | 0.687 | 0.031 | 9.03E−01 | 0.00E+00 | 0.00E+00 |
| Napsa | 2 | 7 | 0.686 | 0.019 | 9.01E−01 | 0.00E+00 | 0.00E+00 |
| Fcer2a | 2 | 7 | 0.613 | 0.018 | 8.60E−01 | 0.00E+00 | 0.00E+00 |
| Irf8 | 2 | 7 | 0.701 | 0.07 | 8.56E−01 | 0.00E+00 | 0.00E+00 |
| Cd24a | 2 | 7 | 0.59 | 0.063 | 7.87E−01 | 0.00E+00 | 0.00E+00 |
| Cd72 | 2 | 7 | 0.628 | 0.067 | 7.60E−01 | 0.00E+00 | 0.00E+00 |
| Bank1 | 2 | 7 | 0.628 | 0.023 | 7.46E−01 | 0.00E+00 | 0.00E+00 |
| Ctsh | 2 | 7 | 0.61 | 0.03 | 7.35E−01 | 0.00E+00 | 0.00E+00 |
| Pkig | 2 | 7 | 0.611 | 0.041 | 7.21E−01 | 0.00E+00 | 0.00E+00 |
| Siglecg | 2 | 7 | 0.569 | 0.012 | 6.82E−01 | 0.00E+00 | 0.00E+00 |
| Fcrla | 2 | 7 | 0.535 | 0.02 | 6.63E−01 | 0.00E+00 | 0.00E+00 |
| Unc93b1 | 2 | 7 | 0.593 | 0.065 | 6.58E−01 | 0.00E+00 | 0.00E+00 |
| H2-Oa | 2 | 7 | 0.545 | 0.094 | 5.88E−01 | 0.00E+00 | 0.00E+00 |
| Snn | 2 | 7 | 0.524 | 0.043 | 5.79E−01 | 0.00E+00 | 0.00E+00 |
| H2-Ob | 2 | 7 | 0.542 | 0.089 | 5.44E−01 | 0.00E+00 | 0.00E+00 |
| Cd79a | 2 | 8 | 0.995 | 0.022 | 2.36E+00 | 0.00E+00 | 0.00E+00 |
| Iglc2 | 2 | 8 | 0.897 | 0.029 | 1.91E+00 | 0.00E+00 | 0.00E+00 |
| Apoe | 2 | 8 | 0.618 | 0.058 | 1.84E+00 | 0.00E+00 | 0.00E+00 |
| H2-DMb2 | 2 | 8 | 0.966 | 0.029 | 1.79E+00 | 0.00E+00 | 0.00E+00 |
| Cd79b | 2 | 8 | 0.952 | 0.025 | 1.78E+00 | 0.00E+00 | 0.00E+00 |
| Ebf1 | 2 | 8 | 0.971 | 0.016 | 1.76E+00 | 0.00E+00 | 0.00E+00 |
| Iglc1 | 2 | 8 | 0.54 | 0.071 | 1.49E+00 | 0.00E+00 | 0.00E+00 |
| Iglc3 | 2 | 8 | 0.886 | 0.04 | 1.47E+00 | 0.00E+00 | 0.00E+00 |
| Ms4a1 | 2 | 8 | 0.876 | 0.008 | 1.38E+00 | 0.00E+00 | 0.00E+00 |
| Fcmr | 2 | 8 | 0.817 | 0.009 | 1.37E+00 | 0.00E+00 | 0.00E+00 |
| Ifi30 | 2 | 8 | 0.787 | 0.058 | 1.21E+00 | 0.00E+00 | 0.00E+00 |
| Ighd | 2 | 8 | 0.738 | 0.016 | 1.15E+00 | 0.00E+00 | 0.00E+00 |
| Mzb1 | 2 | 8 | 0.671 | 0.025 | 1.01E+00 | 0.00E+00 | 0.00E+00 |
| Vpreb3 | 2 | 8 | 0.635 | 0.007 | 9.89E−01 | 0.00E+00 | 0.00E+00 |
| Tnfrsf13c | 2 | 8 | 0.687 | 0.018 | 9.15E−01 | 0.00E+00 | 0.00E+00 |
| Fcer2a | 2 | 8 | 0.613 | 0.004 | 8.75E−01 | 0.00E+00 | 0.00E+00 |
| Cd24a | 2 | 8 | 0.59 | 0.013 | 8.54E−01 | 0.00E+00 | 0.00E+00 |
| Capg | 2 | 8 | 0.635 | 0.089 | 7.74E−01 | 0.00E+00 | 0.00E+00 |
| Bank1 | 2 | 8 | 0.628 | 0.01 | 7.60E−01 | 0.00E+00 | 0.00E+00 |
| Pou2f2 | 2 | 8 | 0.578 | 0.049 | 6.98E−01 | 0.00E+00 | 0.00E+00 |
| Siglecg | 2 | 8 | 0.569 | 0.013 | 6.81E−01 | 0.00E+00 | 0.00E+00 |
| Chchd10 | 2 | 8 | 0.549 | 0.07 | 6.79E−01 | 0.00E+00 | 0.00E+00 |
| Ctsh | 2 | 8 | 0.61 | 0.087 | 6.76E−01 | 0.00E+00 | 0.00E+00 |
| Fcrla | 2 | 8 | 0.535 | 0.015 | 6.69E−01 | 0.00E+00 | 0.00E+00 |
| H2-Ob | 2 | 8 | 0.542 | 0.01 | 6.27E−01 | 0.00E+00 | 0.00E+00 |
| H2-Oa | 2 | 8 | 0.545 | 0.062 | 6.24E−01 | 0.00E+00 | 0.00E+00 |
| Snn | 2 | 8 | 0.524 | 0.013 | 6.09E−01 | 0.00E+00 | 0.00E+00 |
| S100a10 | 2 | 9 | 0.557 | 0.093 | 6.13E−01 | 5.99E−251 | 1.06E−243 |
| Ighd | 2 | 9 | 0.738 | 0.067 | 1.08E+00 | 6.91E−174 | 1.22E−166 |
| Ighd | 2 | 10 | 0.738 | 0.046 | 1.09E+00 | 0.00E+00 | 0.00E+00 |
| S100a10 | 2 | 10 | 0.557 | 0.053 | 6.71E−01 | 0.00E+00 | 0.00E+00 |
| S100a11 | 2 | 10 | 0.534 | 0.09 | 5.49E−01 | 0.00E+00 | 0.00E+00 |
| Ccr7 | 2 | 10 | 0.544 | 0.069 | 8.35E−01 | 4.09E−304 | 7.25E−297 |
| Cd79a | 2 | 11 | 0.995 | 0.019 | 2.38E+00 | 0.00E+00 | 0.00E+00 |
| Iglc2 | 2 | 11 | 0.897 | 0.027 | 1.91E+00 | 0.00E+00 | 0.00E+00 |
| H2-DMb2 | 2 | 11 | 0.966 | 0.036 | 1.79E+00 | 0.00E+00 | 0.00E+00 |
| Ebf1 | 2 | 11 | 0.971 | 0.004 | 1.78E+00 | 0.00E+00 | 0.00E+00 |
| Cd79b | 2 | 11 | 0.952 | 0.048 | 1.76E+00 | 0.00E+00 | 0.00E+00 |
| Iglc3 | 2 | 11 | 0.886 | 0.024 | 1.56E+00 | 0.00E+00 | 0.00E+00 |
| Ms4a1 | 2 | 11 | 0.876 | 0.007 | 1.39E+00 | 0.00E+00 | 0.00E+00 |
| Fcmr | 2 | 11 | 0.817 | 0.004 | 1.38E+00 | 0.00E+00 | 0.00E+00 |
| Ifi30 | 2 | 11 | 0.787 | 0.064 | 1.21E+00 | 0.00E+00 | 0.00E+00 |
| Mef2c | 2 | 11 | 0.811 | 0.048 | 1.17E+00 | 0.00E+00 | 0.00E+00 |
| Ighd | 2 | 11 | 0.738 | 0.008 | 1.16E+00 | 0.00E+00 | 0.00E+00 |
| Mzb1 | 2 | 11 | 0.671 | 0.029 | 1.01E+00 | 0.00E+00 | 0.00E+00 |
| Vpreb3 | 2 | 11 | 0.635 | 0.012 | 9.88E−01 | 0.00E+00 | 0.00E+00 |
| Tnfrsf13c | 2 | 11 | 0.687 | 0.016 | 9.17E−01 | 0.00E+00 | 0.00E+00 |
| Bank1 | 2 | 11 | 0.628 | 0.001 | 7.69E−01 | 0.00E+00 | 0.00E+00 |
| Fcer2a | 2 | 11 | 0.613 | 0.004 | 8.77E−01 | 4.1E−315 | 7.22E−308 |
| Pkig | 2 | 11 | 0.611 | 0.055 | 7.09E−01 | 1.75E−306 | 3.09E−299 |
| Apoe | 2 | 11 | 0.618 | 0.037 | 1.86E+00 | 1.07E−298 | 1.90E−291 |
| Siglecg | 2 | 11 | 0.569 | 0.01 | 6.85E−01 | 5.52E−298 | 9.77E−291 |
| Ctsh | 2 | 11 | 0.61 | 0.07 | 6.93E−01 | 1.24E−285 | 2.19E−278 |
| H2-Ob | 2 | 11 | 0.542 | 0.007 | 6.30E−01 | 1.78E−281 | 3.16E−274 |
| Snn | 2 | 11 | 0.524 | 0.007 | 6.17E−01 | 4.96E−272 | 8.78E−265 |
| Fcrla | 2 | 11 | 0.535 | 0.017 | 6.67E−01 | 2.76E−271 | 4.89E−264 |
| Iglc1 | 2 | 11 | 0.54 | 0.05 | 1.52E+00 | 3.25E−257 | 5.76E−250 |
| H2-Oa | 2 | 11 | 0.545 | 0.039 | 6.53E−01 | 9.48E−246 | 1.68E−238 |
| Ccr7 | 2 | 11 | 0.544 | 0.035 | 8.84E−01 | 2.87E−242 | 5.09E−235 |
| Chchd10 | 2 | 11 | 0.549 | 0.058 | 6.90E−01 | 1.12E−236 | 1.99E−229 |
| Pou2f2 | 2 | 11 | 0.578 | 0.092 | 6.49E−01 | 1.25E−223 | 2.21E−216 |
| Cd79a | 2 | 12 | 0.995 | 0.036 | 2.36E+00 | 0.00E+00 | 0.00E+00 |
| Iglc2 | 2 | 12 | 0.897 | 0.037 | 1.90E+00 | 0.00E+00 | 0.00E+00 |
| H2-DMb2 | 2 | 12 | 0.966 | 0.043 | 1.78E+00 | 0.00E+00 | 0.00E+00 |
| Ebf1 | 2 | 12 | 0.971 | 0.009 | 1.77E+00 | 0.00E+00 | 0.00E+00 |
| Iglc3 | 2 | 12 | 0.886 | 0.019 | 1.56E+00 | 0.00E+00 | 0.00E+00 |
| Ms4a1 | 2 | 12 | 0.876 | 0.02 | 1.37E+00 | 0.00E+00 | 0.00E+00 |
| Fcmr | 2 | 12 | 0.817 | 0.012 | 1.37E+00 | 0.00E+00 | 0.00E+00 |
| Mef2c | 2 | 12 | 0.811 | 0.021 | 1.20E+00 | 0.00E+00 | 0.00E+00 |
| Ifi30 | 2 | 12 | 0.787 | 0.069 | 1.20E+00 | 0.00E+00 | 0.00E+00 |
| Ighd | 2 | 12 | 0.738 | 0.005 | 1.17E+00 | 0.00E+00 | 0.00E+00 |
| Mzb1 | 2 | 12 | 0.671 | 0.034 | 1.00E+00 | 0.00E+00 | 0.00E+00 |
| Vpreb3 | 2 | 12 | 0.635 | 0.003 | 9.96E−01 | 0.00E+00 | 0.00E+00 |
| Tnfrsf13c | 2 | 12 | 0.687 | 0.009 | 9.26E−01 | 0.00E+00 | 0.00E+00 |
| Napsa | 2 | 12 | 0.686 | 0.068 | 8.50E−01 | 0.00E+00 | 0.00E+00 |
| Bank1 | 2 | 12 | 0.628 | 0.003 | 7.67E−01 | 0.00E+00 | 6.42E−302 |
| Pkig | 2 | 12 | 0.611 | 0.064 | 6.98E−01 | 7.21E−288 | 1.28E−280 |
| Fcer2a | 2 | 12 | 0.613 | 0.008 | 8.74E−01 | 8.49E−284 | 1.50E−276 |
| Ly6e | 2 | 12 | 0.673 | 0.088 | 1.03E+00 | 2.85E−281 | 5.05E−274 |
| Siglecg | 2 | 12 | 0.569 | 0.012 | 6.83E−01 | 2.14E−275 | 3.79E−268 |
| Apoe | 2 | 12 | 0.618 | 0.053 | 1.84E+00 | 1.70E−267 | 3.01E−260 |
| Fcrla | 2 | 12 | 0.535 | 0.012 | 6.72E−01 | 4.54E−263 | 8.05E−256 |
| H2-Ob | 2 | 12 | 0.542 | 0.01 | 6.27E−01 | 5.35E−258 | 9.48E−251 |
| Ctsh | 2 | 12 | 0.61 | 0.098 | 6.63E−01 | 3.85E−247 | 6.81E−240 |
| Snn | 2 | 12 | 0.524 | 0.012 | 6.11E−01 | 3.77E−245 | 6.68E−238 |
| Ctss | 2 | 12 | 0.601 | 0.087 | 6.12E−01 | 8.84E−239 | 1.57E−231 |
| Cd24a | 2 | 12 | 0.59 | 0.069 | 7.66E−01 | 2.56E−238 | 4.53E−231 |
| Iglc1 | 2 | 12 | 0.54 | 0.07 | 1.50E+00 | 5.13E−224 | 9.09E−217 |
| Chchd10 | 2 | 12 | 0.549 | 0.067 | 6.81E−01 | 8.46E−217 | 1.50E−209 |
| Ccr7 | 2 | 12 | 0.544 | 0.047 | 8.53E−01 | 7.54E−211 | 1.33E−203 |
| Ighd | 2 | 13 | 0.738 | 0.006 | 1.17E+00 | 9.31E−288 | 1.65E−280 |
| H2-Aa | 2 | 13 | 0.994 | 0.076 | 3.19E+00 | 1.58E−267 | 2.80E−260 |
| Mef2c | 2 | 13 | 0.811 | 0.009 | 1.22E+00 | 8.09E−185 | 1.43E−177 |
| H2-Eb1 | 2 | 13 | 0.989 | 0.059 | 2.65E+00 | 2.70E−178 | 4.77E−171 |
| Fcmr | 2 | 13 | 0.817 | 0.014 | 1.37E+00 | 3.39E−176 | 6.01E−169 |
| Fcer2a | 2 | 13 | 0.613 | 0.007 | 8.74E−01 | 1.56E−175 | 2.75E−168 |
| Ebf1 | 2 | 13 | 0.971 | 0.004 | 1.78E+00 | 4.68E−172 | 8.29E−165 |
| Bank1 | 2 | 13 | 0.628 | 0.004 | 7.66E−01 | 7.24E−166 | 1.28E−158 |
| Cd79a | 2 | 14 | 0.995 | 0.081 | 2.25E+00 | 0.00E+00 | 0.00E+00 |
| Ebf1 | 2 | 14 | 0.971 | 0.034 | 1.71E+00 | 2.31E−299 | 4.09E−292 |
| Ms4a1 | 2 | 14 | 0.876 | 0.053 | 1.31E+00 | 1.80E−295 | 3.19E−288 |
| Iglc2 | 2 | 14 | 0.897 | 0.063 | 1.79E+00 | 5.45E−295 | 9.65E−288 |
| Mzb1 | 2 | 14 | 0.671 | 0.094 | 8.58E−01 | 1.05E−239 | 1.86E−232 |
| Fcmr | 2 | 14 | 0.817 | 0.023 | 1.34E+00 | 2.25E−231 | 3.98E−224 |
| Tnfrsf13c | 2 | 14 | 0.687 | 0.091 | 8.31E−01 | 1.51E−209 | 2.67E−202 |
| Vpreb3 | 2 | 14 | 0.635 | 0.014 | 9.81E−01 | 1.89E−184 | 3.34E−177 |
| Chchd10 | 2 | 14 | 0.549 | 0.085 | 6.44E−01 | 4.45E−175 | 7.87E−168 |
| Fcrla | 2 | 14 | 0.535 | 0.061 | 6.16E−01 | 3.76E−165 | 6.66E−158 |
| Cd79a | 2 | 15 | 0.995 | 0.021 | 2.38E+00 | 0.00E+00 | 0.00E+00 |
| Ighm | 2 | 15 | 0.923 | 0.07 | 2.19E+00 | 9.9E−315 | 1.75E−307 |
| Ebf1 | 2 | 15 | 0.971 | 0.013 | 1.76E+00 | 9.9E−310 | 1.77E−302 |
| H2-DMb2 | 2 | 15 | 0.966 | 0.027 | 1.80E+00 | 2.09E−279 | 3.70E−272 |
| Ighd | 2 | 15 | 0.738 | 0 | 1.17E+00 | 7.89E−252 | 1.40E−244 |
| Cd79b | 2 | 15 | 0.952 | 0.061 | 1.74E+00 | 4.07E−241 | 7.21E−234 |
| Iglc2 | 2 | 15 | 0.897 | 0.019 | 1.92E+00 | 5.23E−228 | 9.27E−221 |
| Ms4a1 | 2 | 15 | 0.876 | 0 | 1.39E+00 | 7.94E−214 | 1.41E−206 |
| Fcmr | 2 | 15 | 0.817 | 0.008 | 1.37E+00 | 9.13E−206 | 1.62E−198 |
| Iglc3 | 2 | 15 | 0.886 | 0.006 | 1.58E+00 | 4.90E−205 | 8.68E−198 |
| Mef2c | 2 | 15 | 0.811 | 0.038 | 1.19E+00 | 1.72E−183 | 3.05E−176 |
| Ighm | 2 | 16 | 0.923 | 0.049 | 2.20E+00 | 3.66E−252 | 6.49E−245 |
| Cd79a | 2 | 16 | 0.995 | 0.028 | 2.37E+00 | 2.35E−217 | 4.16E−210 |
| Ighd | 2 | 16 | 0.738 | 0.008 | 1.16E+00 | 1.14E−200 | 2.02E−193 |
| Ebf1 | 2 | 16 | 0.971 | 0.036 | 1.73E+00 | 4.09E−185 | 7.24E−178 |
| H2-DMb2 | 2 | 16 | 0.966 | 0.015 | 1.81E+00 | 1.88E−177 | 3.32E−170 |
| Cd79a | 2 | 17 | 0.995 | 0.082 | 2.30E+00 | 0.00E+00 | 0.00E+00 |
| H2-DMb2 | 2 | 17 | 0.966 | 0.059 | 1.76E+00 | 0.00E+00 | 0.00E+00 |
| Ebf1 | 2 | 17 | 0.971 | 0.018 | 1.75E+00 | 0.00E+00 | 0.00E+00 |
| Iglc3 | 2 | 17 | 0.886 | 0.035 | 1.54E+00 | 7.17E−291 | 1.27E−283 |
| Ms4a1 | 2 | 17 | 0.876 | 0.035 | 1.36E+00 | 5.25E−281 | 9.30E−274 |
| Iglc2 | 2 | 17 | 0.897 | 0.05 | 1.88E+00 | 4.90E−280 | 8.68E−273 |
| Fcmr | 2 | 17 | 0.817 | 0.021 | 1.36E+00 | 1.61E−219 | 2.85E−212 |
| Mef2c | 2 | 17 | 0.811 | 0.041 | 1.17E+00 | 3.32E−204 | 5.88E−197 |
| Cd83 | 2 | 17 | 0.735 | 0.065 | 1.16E+00 | 1.16E−168 | 2.06E−161 |
| Tnfrsf13c | 2 | 17 | 0.687 | 0.038 | 8.98E−01 | 1.02E−163 | 1.80E−156 |
| Ebf1 | 2 | 18 | 0.971 | 0.036 | 1.74E+00 | 0.00E+00 | 0.00E+00 |
| Iglc3 | 2 | 18 | 0.886 | 0.053 | 1.53E+00 | 1.07E−244 | 1.89E−237 |
| Iglc2 | 2 | 18 | 0.897 | 0.085 | 1.85E+00 | 2.09E−227 | 3.71E−220 |
| Ms4a1 | 2 | 18 | 0.876 | 0.078 | 1.31E+00 | 6.40E−223 | 1.13E−215 |
| Mef2c | 2 | 18 | 0.811 | 0.021 | 1.21E+00 | 1.58E−182 | 2.79E−175 |
| Fcmr | 2 | 18 | 0.817 | 0.039 | 1.34E+00 | 2.44E−182 | 4.32E−175 |
| Ifi30 | 2 | 18 | 0.787 | 0.078 | 1.19E+00 | 1.51E−169 | 2.68E−162 |
| Cd79a | 2 | 19 | 0.995 | 0.072 | 2.33E+00 | 0.00E+00 | 0.00E+00 |
| Ebf1 | 2 | 19 | 0.971 | 0.016 | 1.76E+00 | 8.53E−302 | 1.51E−294 |
| H2-DMb2 | 2 | 19 | 0.966 | 0.04 | 1.78E+00 | 1.62E−288 | 2.88E−281 |
| Cd79b | 2 | 19 | 0.952 | 0.064 | 1.74E+00 | 1.02E−252 | 1.81E−245 |
| Iglc3 | 2 | 19 | 0.886 | 0.02 | 1.56E+00 | 2.01E−206 | 3.55E−199 |
| Iglc2 | 2 | 19 | 0.897 | 0.036 | 1.90E+00 | 7.14E−204 | 1.26E−196 |
| Ms4a1 | 2 | 19 | 0.876 | 0.028 | 1.37E+00 | 8.31E−192 | 1.47E−184 |
| Fcmr | 2 | 19 | 0.817 | 0.012 | 1.37E+00 | 1.78E−162 | 3.16E−155 |
| Cd79a | 2 | 21 | 0.995 | 0.098 | 2.28E+00 | 3.60E−235 | 6.37E−228 |
| Iglc3 | 2 | 21 | 0.886 | 0.081 | 1.49E+00 | 3.39E−181 | 5.99E−174 |
| Ms4a1 | 2 | 21 | 0.876 | 0.034 | 1.36E+00 | 3.37E−174 | 5.96E−167 |
| Cd79a | 2 | 22 | 0.995 | 0.06 | 2.27E+00 | 1.90E−282 | 3.36E−275 |
| Ebf1 | 2 | 22 | 0.971 | 0.043 | 1.70E+00 | 9.75E−224 | 1.73E−216 |
| Iglc3 | 2 | 22 | 0.886 | 0.069 | 1.49E+00 | 1.76E−207 | 3.11E−200 |
| Ms4a1 | 2 | 22 | 0.876 | 0.039 | 1.34E+00 | 3.73E−194 | 6.61E−187 |
| Iglc2 | 2 | 22 | 0.897 | 0.056 | 1.80E+00 | 5.88E−186 | 1.04E−178 |
| Cd79a | 2 | 24 | 0.995 | 0.049 | 2.35E+00 | 1.67E−304 | 2.95E−297 |
| Iglc3 | 2 | 24 | 0.886 | 0.081 | 1.45E+00 | 9.13E−201 | 1.62E−193 |
| Ms4a1 | 2 | 24 | 0.876 | 0.045 | 1.35E+00 | 7.25E−194 | 1.28E−186 |
| Iglc2 | 2 | 24 | 0.897 | 0.04 | 1.89E+00 | 2.69E−190 | 4.77E−183 |
| Cd79a | 2 | 25 | 0.995 | 0.027 | 2.38E+00 | 0.00E+00 | 0.00E+00 |
| H2-DMb2 | 2 | 25 | 0.966 | 0.045 | 1.78E+00 | 8.70E−269 | 1.54E−261 |
| Cd79b | 2 | 25 | 0.952 | 0.009 | 1.80E+00 | 8.96E−260 | 1.59E−252 |
| Coro1a | 2 | 25 | 0.958 | 0.1 | 1.62E+00 | 1.35E−250 | 2.40E−243 |
| Cd37 | 2 | 25 | 0.902 | 0.014 | 1.40E+00 | 1.55E−210 | 2.74E−203 |
| Ptprcap | 2 | 25 | 0.902 | 0.063 | 1.31E+00 | 5.36E−209 | 9.49E−202 |
| Ms4a1 | 2 | 25 | 0.876 | 0 | 1.39E+00 | 4.12E−203 | 7.29E−196 |
| Ighm | 2 | 25 | 0.923 | 0.077 | 2.18E+00 | 2.75E−199 | 4.86E−192 |
| Iglc3 | 2 | 25 | 0.886 | 0.027 | 1.55E+00 | 1.18E−195 | 2.08E−188 |
| Iglc2 | 2 | 25 | 0.897 | 0.036 | 1.90E+00 | 2.77E−191 | 4.90E−184 |
| Fcmr | 2 | 25 | 0.817 | 0 | 1.38E+00 | 5.24E−159 | 9.27E−152 |
| Cd79a | 2 | 26 | 0.995 | 0.018 | 2.38E+00 | 5.72E−297 | 1.01E−289 |
| H2-DMb2 | 2 | 26 | 0.966 | 0.069 | 1.73E+00 | 5.83E−248 | 1.03E−240 |
| Ebf1 | 2 | 26 | 0.971 | 0.005 | 1.78E+00 | 3.67E−244 | 6.50E−237 |
| Cd79b | 2 | 26 | 0.952 | 0.092 | 1.70E+00 | 1.03E−224 | 1.83E−217 |
| Iglc3 | 2 | 26 | 0.886 | 0.069 | 1.47E+00 | 1.20E−205 | 2.13E−198 |
| Ms4a1 | 2 | 26 | 0.876 | 0.014 | 1.38E+00 | 1.91E−200 | 3.38E−193 |
| Iglc2 | 2 | 26 | 0.897 | 0.055 | 1.86E+00 | 3.31E−189 | 5.87E−182 |
| Cd79a | 2 | 30 | 0.995 | 0.052 | 2.32E+00 | 5.67E−240 | l.OOE−232 |
| H2-DMb2 | 2 | 30 | 0.966 | 0.03 | 1.79E+00 | 3.43E−182 | 6.07E−175 |
| Ebf1 | 2 | 30 | 0.971 | 0.022 | 1.76E+00 | 4.17E−181 | 7.38E−174 |
| Cd79b | 2 | 30 | 0.952 | 0.059 | 1.71E+00 | 2.66E−160 | 4.71E−153 |
| Cd79a | 2 | 31 | 0.995 | 0.037 | 2.37E+00 | 2.47E−182 | 4.38E−175 |
| Cd52 | 2 | 32 | 0.991 | 0.094 | 2.51E+00 | 1.96E−209 | 3.48E−202 |
| Cd79a | 2 | 32 | 0.995 | 0.009 | 2.39E+00 | 1.10E−204 | 1.95E−197 |
| Cd52 | 2 | 33 | 0.991 | 0.076 | 2.57E+00 | 3.71E−173 | 6.56E−166 |
| Cd79a | 2 | 33 | 0.995 | 0.057 | 2.34E+00 | 2.24E−169 | 3.96E−162 |
| Ncr1 | 3 | 4 | 0.709 | 0.093 | 9.33E−01 | 0.00E+00 | 0.00E+00 |
| Tmem176b | 3 | 5 | 0.975 | 0.016 | 1.86E+00 | 0.00E+00 | 0.00E+00 |
| Lgals3 | 3 | 5 | 0.938 | 0.012 | 1.72E+00 | 0.00E+00 | 0.00E+00 |
| Tmem176a | 3 | 5 | 0.952 | 0.018 | 1.69E+00 | 0.00E+00 | 0.00E+00 |
| Car2 | 3 | 5 | 0.915 | 0.041 | 1.57E+00 | 0.00E+00 | 0.00E+00 |
| Fcer1g | 3 | 5 | 0.904 | 0.02 | 1.53E+00 | 0.00E+00 | 0.00E+00 |
| Lmo4 | 3 | 5 | 0.86 | 0.101 | 1.35E+00 | 0.00E+00 | 0.00E+00 |
| Gadd45b | 3 | 5 | 0.621 | 0.051 | 1.32E+00 | 0.00E+00 | 0.00E+00 |
| Serpinb1a | 3 | 5 | 0.777 | 0.01 | 1.31E+00 | 0.00E+00 | 0.00E+00 |
| Gem | 3 | 5 | 0.587 | 0.028 | 1.21E+00 | 0.00E+00 | 0.00E+00 |
| Nr4a1 | 3 | 5 | 0.684 | 0.095 | 1.14E+00 | 0.00E+00 | 0.00E+00 |
| Klrk1 | 3 | 5 | 0.742 | 0.013 | 1.12E+00 | 0.00E+00 | 0.00E+00 |
| Ckb | 3 | 5 | 0.802 | 0.022 | 1.10E+00 | 0.00E+00 | 0.00E+00 |
| Ncr1 | 3 | 5 | 0.709 | 0.002 | 1.05E+00 | 0.00E+00 | 0.00E+00 |
| Bhlhe40 | 3 | 5 | 0.688 | 0.015 | 1.04E+00 | 0.00E+00 | 0.00E+00 |
| Upp1 | 3 | 5 | 0.722 | 0.001 | 1.04E+00 | 0.00E+00 | 0.00E+00 |
| Rora | 3 | 5 | 0.756 | 0.016 | 1.03E+00 | 0.00E+00 | 0.00E+00 |
| Ffar2 | 3 | 5 | 0.693 | 0.002 | 9.97E−01 | 0.00E+00 | 0.00E+00 |
| Ncoa7 | 3 | 5 | 0.687 | 0.048 | 9.89E−01 | 0.00E+00 | 0.00E+00 |
| Tcrg-C1 | 3 | 5 | 0.637 | 0.012 | 9.84E−01 | 0.00E+00 | 0.00E+00 |
| Serpina3g | 3 | 5 | 0.578 | 0.005 | 9.43E−01 | 0.00E+00 | 0.00E+00 |
| Ikzf2 | 3 | 5 | 0.705 | 0.046 | 9.32E−01 | 0.00E+00 | 0.00E+00 |
| St6galnac3 | 3 | 5 | 0.633 | 0.022 | 7.02E−01 | 0.00E+00 | 0.00E+00 |
| Prr29 | 3 | 5 | 0.51 | 0.002 | 6.80E−01 | 0.00E+00 | 0.00E+00 |
| Chad | 3 | 5 | 0.55 | 0.003 | 6.78E−01 | 0.00E+00 | 0.00E+00 |
| Stk19 | 3 | 5 | 0.567 | 0.035 | 6.78E−01 | 0.00E+00 | 0.00E+00 |
| Podnl1 | 3 | 5 | 0.625 | 0.042 | 6.72E−01 | 0.00E+00 | 0.00E+00 |
| Nrgn | 3 | 5 | 0.509 | 0.004 | 6.68E−01 | 0.00E+00 | 0.00E+00 |
| Asb2 | 3 | 5 | 0.507 | 0.006 | 6.62E−01 | 0.00E+00 | 0.00E+00 |
| Maf | 3 | 5 | 0.527 | 0.008 | 6.40E−01 | 0.00E+00 | 0.00E+00 |
| Maff | 3 | 5 | 0.508 | 0.023 | 7.09E−01 | 2.12E−304 | 3.75E−297 |
| Tnfrsf25 | 3 | 5 | 0.527 | 0.034 | 5.52E−01 | 1.58E−289 | 2.79E−282 |
| Cd160 | 3 | 5 | 0.503 | 0.039 | 5.57E−01 | 2.92E−248 | 5.17E−241 |
| Samsn1 | 3 | 5 | 0.531 | 0.06 | 6.13E−01 | 9.53E−242 | 1.69E−234 |
| Nfkbiz | 3 | 5 | 0.511 | 0.074 | 6.23E−01 | 1.15E−204 | 2.03E−197 |
| Capg | 3 | 5 | 0.536 | 0.076 | 6.29E−01 | 2.48E−204 | 4.39E−197 |
| Cxcr6 | 3 | 6 | 0.935 | 0.078 | 1.57E+00 | 0.00E+00 | 0.00E+00 |
| Id2 | 3 | 6 | 0.824 | 0.096 | 1.50E+00 | 0.00E+00 | 0.00E+00 |
| Car2 | 3 | 6 | 0.915 | 0.101 | 1.50E+00 | 0.00E+00 | 0.00E+00 |
| Trbc2 | 3 | 6 | 0.818 | 0.084 | 1.33E+00 | 0.00E+00 | 0.00E+00 |
| Serpinb1a | 3 | 6 | 0.777 | 0.062 | 1.26E+00 | 0.00E+00 | 0.00E+00 |
| Trbc1 | 3 | 6 | 0.862 | 0.07 | 1.24E+00 | 0.00E+00 | 0.00E+00 |
| Upp1 | 3 | 6 | 0.722 | 0.044 | 9.97E−01 | 0.00E+00 | 0.00E+00 |
| Ncr1 | 3 | 6 | 0.709 | 0.067 | 9.84E−01 | 0.00E+00 | 0.00E+00 |
| Ffar2 | 3 | 6 | 0.693 | 0.035 | 9.63E−01 | 0.00E+00 | 0.00E+00 |
| Tcrg-C1 | 3 | 6 | 0.637 | 0.037 | 9.61E−01 | 0.00E+00 | 0.00E+00 |
| Rora | 3 | 6 | 0.756 | 0.09 | 9.48E−01 | 0.00E+00 | 0.00E+00 |
| Txk | 3 | 6 | 0.747 | 0.076 | 8.89E−01 | 0.00E+00 | 0.00E+00 |
| Prr29 | 3 | 6 | 0.51 | 0.02 | 6.62E−01 | 5.50E−298 | 9.75E−291 |
| Gimap7 | 3 | 6 | 0.556 | 0.043 | 6.16E−01 | 6.91E−293 | 1.22E−285 |
| Cd160 | 3 | 6 | 0.503 | 0.021 | 5.76E−01 | 9.52E−293 | 1.69E−285 |
| Serpina3g | 3 | 6 | 0.578 | 0.064 | 8.84E−01 | 4.05E−282 | 7.17E−275 |
| Maf | 3 | 6 | 0.527 | 0.015 | 6.33E−01 | 1.63E−281 | 2.89E−274 |
| Lck | 3 | 6 | 0.584 | 0.098 | 5.60E−01 | 5.02E−279 | 8.89E−272 |
| Nrgn | 3 | 6 | 0.509 | 0.04 | 6.29E−01 | 6.28E−276 | 1.11E−268 |
| Tnfrsf25 | 3 | 6 | 0.527 | 0.027 | 5.58E−01 | 6.33E−269 | 1.12E−261 |
| Ptpn22 | 3 | 6 | 0.552 | 0.057 | 5.86E−01 | 1.22E−268 | 2.16E−261 |
| Asb2 | 3 | 6 | 0.507 | 0.05 | 6.16E−01 | 3.58E−264 | 6.35E−257 |
| Skap1 | 3 | 6 | 0.5 | 0.033 | 5.10E−01 | 6.03E−260 | 1.07E−252 |
| Il18r1 | 3 | 6 | 0.592 | 0.048 | 6.50E−01 | 2.01E−252 | 3.56E−245 |
| Gimap5 | 3 | 6 | 0.5 | 0.058 | 5.21E−01 | 2.77E−245 | 4.90E−238 |
| Chad | 3 | 6 | 0.55 | 0.051 | 6.24E−01 | 1.34E−231 | 2.37E−224 |
| Maff | 3 | 6 | 0.508 | 0.072 | 6.55E−01 | 2.85E−204 | 5.05E−197 |
| Tmem176b | 3 | 7 | 0.975 | 0.031 | 1.83E+00 | 0.00E+00 | 0.00E+00 |
| Lgals3 | 3 | 7 | 0.938 | 0.03 | 1.68E+00 | 0.00E+00 | 0.00E+00 |
| Tmem176a | 3 | 7 | 0.952 | 0.021 | 1.68E+00 | 0.00E+00 | 0.00E+00 |
| Car2 | 3 | 7 | 0.915 | 0.019 | 1.58E+00 | 0.00E+00 | 0.00E+00 |
| Fcer1g | 3 | 7 | 0.904 | 0.019 | 1.53E+00 | 0.00E+00 | 0.00E+00 |
| Cxcr6 | 3 | 7 | 0.935 | 0.099 | 1.50E+00 | 0.00E+00 | 0.00E+00 |
| Serpinb1a | 3 | 7 | 0.777 | 0.022 | 1.30E+00 | 0.00E+00 | 0.00E+00 |
| Gem | 3 | 7 | 0.587 | 0.088 | 1.12E+00 | 0.00E+00 | 0.00E+00 |
| Klrk1 | 3 | 7 | 0.742 | 0.009 | 1.12E+00 | 0.00E+00 | 0.00E+00 |
| Ncr1 | 3 | 7 | 0.709 | 0.006 | 1.05E+00 | 0.00E+00 | 0.00E+00 |
| Upp1 | 3 | 7 | 0.722 | 0.004 | 1.04E+00 | 0.00E+00 | 0.00E+00 |
| Ffar2 | 3 | 7 | 0.693 | 0.014 | 9.79E−01 | 0.00E+00 | 0.00E+00 |
| Tcrg-C1 | 3 | 7 | 0.637 | 0.014 | 9.79E−01 | 0.00E+00 | 0.00E+00 |
| Serpina3g | 3 | 7 | 0.578 | 0.045 | 8.73E−01 | 0.00E+00 | 0.00E+00 |
| Prr29 | 3 | 7 | 0.51 | 0.001 | 6.80E−01 | 0.00E+00 | 0.00E+00 |
| Chad | 3 | 7 | 0.55 | 0.005 | 6.75E−01 | 0.00E+00 | 0.00E+00 |
| Il18r1 | 3 | 7 | 0.592 | 0.034 | 6.64E−01 | 0.00E+00 | 0.00E+00 |
| Stk19 | 3 | 7 | 0.567 | 0.066 | 6.47E−01 | 0.00E+00 | 0.00E+00 |
| Nrgn | 3 | 7 | 0.509 | 0.042 | 5.78E−01 | 0.00E+00 | 0.00E+00 |
| Cd160 | 3 | 7 | 0.503 | 0.08 | 4.93E−01 | 4.88E−261 | 8.64E−254 |
| Runx3 | 3 | 7 | 0.518 | 0.101 | 4.63E−01 | 9.65E−246 | 1.71E−238 |
| Cxcr6 | 3 | 8 | 0.935 | 0.085 | 1.56E+00 | 0.00E+00 | 0.00E+00 |
| Serpinb1a | 3 | 8 | 0.777 | 0.052 | 1.24E+00 | 0.00E+00 | 0.00E+00 |
| Upp1 | 3 | 8 | 0.722 | 0.026 | 1.02E+00 | 0.00E+00 | 0.00E+00 |
| Ffar2 | 3 | 8 | 0.693 | 0.021 | 9.74E−01 | 0.00E+00 | 0.00E+00 |
| Ncoa7 | 3 | 8 | 0.687 | 0.08 | 9.54E−01 | 0.00E+00 | 0.00E+00 |
| Tcrg-C1 | 3 | 8 | 0.637 | 0.036 | 9.43E−01 | 0.00E+00 | 0.00E+00 |
| Prr29 | 3 | 8 | 0.51 | 0.008 | 6.74E−01 | 0.00E+00 | 0.00E+00 |
| Chad | 3 | 8 | 0.55 | 0.042 | 6.37E−01 | 0.00E+00 | 0.00E+00 |
| Tnfrsf25 | 3 | 8 | 0.527 | 0.048 | 5.34E−01 | 1.65E−292 | 2.92E−285 |
| Stk19 | 3 | 8 | 0.567 | 0.08 | 6.28E−01 | 6.16E−288 | 1.09E−280 |
| Maf | 3 | 8 | 0.527 | 0.079 | 5.54E−01 | 1.24E−241 | 2.20E−234 |
| Capg | 3 | 8 | 0.536 | 0.089 | 6.01E−01 | 5.04E−241 | 8.92E−234 |
| Asb2 | 3 | 8 | 0.507 | 0.088 | 5.67E−01 | 3.60E−224 | 6.37E−217 |
| Tmem176b | 3 | 9 | 0.975 | 0.039 | 1.83E+00 | 0.00E+00 | 0.00E+00 |
| Lgals3 | 3 | 9 | 0.938 | 0.035 | 1.68E+00 | 0.00E+00 | 0.00E+00 |
| Tmem176a | 3 | 9 | 0.952 | 0.034 | 1.67E+00 | 0.00E+00 | 0.00E+00 |
| Cxcr6 | 3 | 9 | 0.935 | 0.017 | 1.64E+00 | 0.00E+00 | 0.00E+00 |
| Fcer1g | 3 | 9 | 0.904 | 0.038 | 1.51E+00 | 0.00E+00 | 0.00E+00 |
| Il7r | 3 | 9 | 0.911 | 0.043 | 1.48E+00 | 0.00E+00 | 0.00E+00 |
| S100a10 | 3 | 9 | 0.914 | 0.093 | 1.29E+00 | 0.00E+00 | 0.00E+00 |
| Ckb | 3 | 9 | 0.802 | 0.052 | 1.05E+00 | 0.00E+00 | 0.00E+00 |
| Serpinb1a | 3 | 9 | 0.777 | 0.067 | 1.22E+00 | 1.71E−278 | 3.03E−271 |
| Ctsw | 3 | 9 | 0.705 | 0.04 | 8.99E−01 | 9.95E−276 | 1.76E−268 |
| Ncr1 | 3 | 9 | 0.709 | 0.008 | 1.05E+00 | 2.71E−258 | 4.80E−251 |
| Txk | 3 | 9 | 0.747 | 0.055 | 9.10E−01 | 1.58E−257 | 2.79E−250 |
| Upp1 | 3 | 9 | 0.722 | 0.015 | 1.03E+00 | 3.55E−250 | 6.29E−243 |
| Cd7 | 3 | 9 | 0.733 | 0.077 | 1.31E+00 | 1.84E−249 | 3.27E−242 |
| Rora | 3 | 9 | 0.756 | 0.013 | 1.04E+00 | 1.38E−247 | 2.45E−240 |
| Tcrg-C1 | 3 | 9 | 0.637 | 0.013 | 9.88E−01 | 8.95E−242 | 1.58E−234 |
| Klrk1 | 3 | 9 | 0.742 | 0.005 | 1.13E+00 | 1.02E−238 | 1.81E−231 |
| Ffar2 | 3 | 9 | 0.693 | 0.014 | 9.81E−01 | 3.26E−234 | 5.77E−227 |
| Bcl2 | 3 | 9 | 0.602 | 0.095 | 8.37E−01 | 1.69E−220 | 2.99E−213 |
| Sla | 3 | 9 | 0.629 | 0.063 | 8.19E−01 | 3.88E−220 | 6.88E−213 |
| Bhlhe40 | 3 | 9 | 0.688 | 0.05 | 9.97E−01 | 7.34E−214 | 1.30E−206 |
| Il2rb | 3 | 9 | 0.514 | 0.041 | 5.57E−01 | 3.47E−213 | 6.14E−206 |
| Gpr183 | 3 | 9 | 0.635 | 0.07 | 7.46E−01 | 2.56E−212 | 4.53E−205 |
| Podnl1 | 3 | 9 | 0.625 | 0.016 | 7.00E−01 | 1.20E−211 | 2.13E−204 |
| Ikzf2 | 3 | 9 | 0.705 | 0.015 | 9.81E−01 | 9.17E−205 | 1.62E−197 |
| Prr29 | 3 | 9 | 0.51 | 0.008 | 6.73E−01 | 1.19E−202 | 2.11E−195 |
| Cd160 | 3 | 9 | 0.503 | 0.006 | 5.92E−01 | 2.80E−193 | 4.96E−186 |
| Serpina3g | 3 | 9 | 0.578 | 0.021 | 9.25E−01 | 3.16E−191 | 5.60E−184 |
| Nabp1 | 3 | 9 | 0.569 | 0.082 | 7.92E−01 | 1.76E−189 | 3.11E−182 |
| Ptpn22 | 3 | 9 | 0.552 | 0.038 | 6.06E−01 | 1.34E−186 | 2.37E−179 |
| Tnfrsf25 | 3 | 9 | 0.527 | 0.012 | 5.74E−01 | 7.36E−183 | 1.30E−175 |
| St6galnac3 | 3 | 9 | 0.633 | 0.019 | 7.05E−01 | 8.09E−183 | 1.43E−175 |
| Asb2 | 3 | 9 | 0.507 | 0.025 | 6.37E−01 | 9.64E−182 | 1.71E−174 |
| Il18r1 | 3 | 9 | 0.592 | 0.009 | 6.95E−01 | 1.07E−168 | 1.89E−161 |
| Maf | 3 | 9 | 0.527 | 0.016 | 6.27E−01 | 4.18E−166 | 7.39E−159 |
| Runx3 | 3 | 9 | 0.518 | 0.097 | 4.50E−01 | 3.68E−162 | 6.51E−155 |
| Maff | 3 | 9 | 0.508 | 0.015 | 7.17E−01 | 5.96E−161 | 1.05E−153 |
| Tmem176b | 3 | 10 | 0.975 | 0.025 | 1.83E+00 | 0.00E+00 | 0.00E+00 |
| Lgals3 | 3 | 10 | 0.938 | 0.03 | 1.70E+00 | 0.00E+00 | 0.00E+00 |
| Tmem176a | 3 | 10 | 0.952 | 0.023 | 1.67E+00 | 0.00E+00 | 0.00E+00 |
| Cxcr6 | 3 | 10 | 0.935 | 0.013 | 1.64E+00 | 0.00E+00 | 0.00E+00 |
| Id2 | 3 | 10 | 0.824 | 0.051 | 1.56E+00 | 0.00E+00 | 0.00E+00 |
| Fcer1g | 3 | 10 | 0.904 | 0.032 | 1.51E+00 | 0.00E+00 | 0.00E+00 |
| Car2 | 3 | 10 | 0.915 | 0.1 | 1.50E+00 | 0.00E+00 | 0.00E+00 |
| Il7r | 3 | 10 | 0.911 | 0.026 | 1.49E+00 | 0.00E+00 | 0.00E+00 |
| Emb | 3 | 10 | 0.914 | 0.057 | 1.37E+00 | 0.00E+00 | 0.00E+00 |
| Sepp1 | 3 | 10 | 0.904 | 0.075 | 1.36E+00 | 0.00E+00 | 0.00E+00 |
| S100a10 | 3 | 10 | 0.914 | 0.053 | 1.35E+00 | 0.00E+00 | 0.00E+00 |
| Cd7 | 3 | 10 | 0.733 | 0.043 | 1.34E+00 | 0.00E+00 | 0.00E+00 |
| Selplg | 3 | 10 | 0.901 | 0.09 | 1.28E+00 | 0.00E+00 | 0.00E+00 |
| Trbc1 | 3 | 10 | 0.862 | 0.064 | 1.27E+00 | 0.00E+00 | 0.00E+00 |
| Hcst | 3 | 10 | 0.872 | 0.089 | 1.27E+00 | 0.00E+00 | 0.00E+00 |
| Serpinb1a | 3 | 10 | 0.777 | 0.088 | 1.18E+00 | 0.00E+00 | 0.00E+00 |
| Klrk1 | 3 | 10 | 0.742 | 0.005 | 1.13E+00 | 0.00E+00 | 0.00E+00 |
| Nr4a1 | 3 | 10 | 0.684 | 0.093 | 1.12E+00 | 0.00E+00 | 0.00E+00 |
| Ckb | 3 | 10 | 0.802 | 0.027 | 1.09E+00 | 0.00E+00 | 0.00E+00 |
| Ncr1 | 3 | 10 | 0.709 | 0.009 | 1.05E+00 | 0.00E+00 | 0.00E+00 |
| Rora | 3 | 10 | 0.756 | 0.008 | 1.04E+00 | 0.00E+00 | 0.00E+00 |
| Upp1 | 3 | 10 | 0.722 | 0.009 | 1.04E+00 | 0.00E+00 | 0.00E+00 |
| Vps37b | 3 | 10 | 0.607 | 0.074 | 1.03E+00 | 0.00E+00 | 0.00E+00 |
| Bhlhe40 | 3 | 10 | 0.688 | 0.04 | 1.00E+00 | 0.00E+00 | 0.00E+00 |
| Tcrg-C1 | 3 | 10 | 0.637 | 0.009 | 9.92E−01 | 0.00E+00 | 0.00E+00 |
| Ikzf2 | 3 | 10 | 0.705 | 0.009 | 9.89E−01 | 0.00E+00 | 0.00E+00 |
| Ffar2 | 3 | 10 | 0.693 | 0.009 | 9.88E−01 | 0.00E+00 | 0.00E+00 |
| Ncoa7 | 3 | 10 | 0.687 | 0.074 | 9.61E−01 | 0.00E+00 | 0.00E+00 |
| Txk | 3 | 10 | 0.747 | 0.03 | 9.42E−01 | 0.00E+00 | 0.00E+00 |
| Serpina3g | 3 | 10 | 0.578 | 0.014 | 9.34E−01 | 0.00E+00 | 0.00E+00 |
| Ctsw | 3 | 10 | 0.705 | 0.018 | 9.21E−01 | 0.00E+00 | 0.00E+00 |
| Bcl2 | 3 | 10 | 0.602 | 0.05 | 8.89E−01 | 0.00E+00 | 0.00E+00 |
| Nabp1 | 3 | 10 | 0.569 | 0.04 | 8.35E−01 | 0.00E+00 | 0.00E+00 |
| Sla | 3 | 10 | 0.629 | 0.05 | 8.30E−01 | 0.00E+00 | 0.00E+00 |
| S100a11 | 3 | 10 | 0.707 | 0.09 | 8.06E−01 | 0.00E+00 | 0.00E+00 |
| Gpr183 | 3 | 10 | 0.635 | 0.048 | 7.69E−01 | 0.00E+00 | 0.00E+00 |
| Maff | 3 | 10 | 0.508 | 0.009 | 7.23E−01 | 0.00E+00 | 0.00E+00 |
| Podnl1 | 3 | 10 | 0.625 | 0.008 | 7.07E−01 | 0.00E+00 | 0.00E+00 |
| St6galnac3 | 3 | 10 | 0.633 | 0.019 | 7.04E−01 | 0.00E+00 | 0.00E+00 |
| Il18r1 | 3 | 10 | 0.592 | 0.005 | 6.98E−01 | 0.00E+00 | 0.00E+00 |
| Prr29 | 3 | 10 | 0.51 | 0.007 | 6.75E−01 | 0.00E+00 | 0.00E+00 |
| Chad | 3 | 10 | 0.55 | 0.005 | 6.73E−01 | 0.00E+00 | 0.00E+00 |
| Asb2 | 3 | 10 | 0.507 | 0.01 | 6.57E−01 | 0.00E+00 | 0.00E+00 |
| Maf | 3 | 10 | 0.527 | 0.01 | 6.39E−01 | 0.00E+00 | 0.00E+00 |
| Ptpn22 | 3 | 10 | 0.552 | 0.024 | 6.18E−01 | 0.00E+00 | 0.00E+00 |
| Cd160 | 3 | 10 | 0.503 | 0.005 | 5.94E−01 | 0.00E+00 | 0.00E+00 |
| Il2rb | 3 | 10 | 0.514 | 0.021 | 5.82E−01 | 0.00E+00 | 0.00E+00 |
| Tnfrsf25 | 3 | 10 | 0.527 | 0.011 | 5.76E−01 | 0.00E+00 | 0.00E+00 |
| Skap1 | 3 | 10 | 0.5 | 0.035 | 5.09E−01 | 4.43E−306 | 7.85E−299 |
| Gpr132 | 3 | 10 | 0.506 | 0.025 | 5.69E−01 | 1.88E−305 | 3.32E−298 |
| Ccnd2 | 3 | 10 | 0.501 | 0.044 | 5.65E−01 | 2.22E−282 | 3.93E−275 |
| Tnfaip3 | 3 | 10 | 0.518 | 0.07 | 6.70E−01 | 3.65E−282 | 6.46E−275 |
| Runx3 | 3 | 10 | 0.518 | 0.049 | 5.27E−01 | 3.49E−277 | 6.17E−270 |
| Ahcyl2 | 3 | 10 | 0.584 | 0.095 | 5.88E−01 | 1.87E−273 | 3.31E−266 |
| Nfkbiz | 3 | 10 | 0.511 | 0.082 | 6.08E−01 | 2.20E−254 | 3.90E−247 |
| Prr29 | 3 | 11 | 0.51 | 0.026 | 6.44E−01 | 2.46E−199 | 4.35E−192 |
| Car2 | 3 | 12 | 0.915 | 0.1 | 1.48E+00 | 0.00E+00 | 0.00E+00 |
| Klrk1 | 3 | 12 | 0.742 | 0.059 | 1.06E+00 | 0.00E+00 | 0.00E+00 |
| Upp1 | 3 | 12 | 0.722 | 0.039 | 1.00E+00 | 0.00E+00 | 0.00E+00 |
| Ncr1 | 3 | 12 | 0.709 | 0.076 | 9.46E−01 | 2.27E−278 | 4.03E−271 |
| Ncoa7 | 3 | 12 | 0.687 | 0.096 | 9.31E−01 | 3.69E−246 | 6.53E−239 |
| Il18r1 | 3 | 12 | 0.592 | 0.07 | 6.10E−01 | 8.42E−191 | 1.49E−183 |
| Chad | 3 | 12 | 0.55 | 0.072 | 6.07E−01 | 5.93E−169 | 1.05E−161 |
| Prr29 | 3 | 12 | 0.51 | 0.054 | 6.21E−01 | 3.24E−165 | 5.74E−158 |
| Cxcr6 | 3 | 14 | 0.935 | 0.091 | 1.52E+00 | 1.09E−274 | 1.92E−267 |
| Upp1 | 3 | 14 | 0.722 | 0.052 | 9.88E−01 | 3.46E−173 | 6.12E−166 |
| Ncr1 | 3 | 14 | 0.709 | 0.077 | 9.39E−01 | 2.37E−171 | 4.20E−164 |
| Tmem176b | 3 | 17 | 0.975 | 0.094 | 1.74E+00 | 0.00E+00 | 0.00E+00 |
| Serpinb1a | 3 | 17 | 0.777 | 0.026 | 1.30E+00 | 5.39E−195 | 9.53E−188 |
| Upp1 | 3 | 17 | 0.722 | 0.009 | 1.04E+00 | 4.52E−171 | 8.01E−164 |
| Ffar2 | 3 | 17 | 0.693 | 0.009 | 9.78E−01 | 3.09E−158 | 5.47E−151 |
| Tmem176b | 3 | 18 | 0.975 | 0.032 | 1.84E+00 | 0.00E+00 | 0.00E+00 |
| Tmem176a | 3 | 18 | 0.952 | 0.011 | 1.69E+00 | 7.52E−299 | 1.33E−291 |
| Lgals3 | 3 | 18 | 0.938 | 0.025 | 1.71E+00 | 4.73E−275 | 8.37E−268 |
| Cxcr6 | 3 | 18 | 0.935 | 0.06 | 1.57E+00 | 1.03E−253 | 1.82E−246 |
| Car2 | 3 | 18 | 0.915 | 0.028 | 1.59E+00 | 1.22E−247 | 2.17E−240 |
| Fcer1g | 3 | 18 | 0.904 | 0.05 | 1.50E+00 | 2.29E−229 | 4.06E−222 |
| Lmo4 | 3 | 18 | 0.86 | 0.089 | 1.36E+00 | 3.31E−171 | 5.86E−164 |
| Serpinb1a | 3 | 18 | 0.777 | 0.018 | 1.31E+00 | 5.90E−164 | 1.04E−156 |
| Tmem176b | 3 | 19 | 0.975 | 0.096 | 1.77E+00 | 8.87E−200 | 1.57E−192 |
| Tmem176a | 3 | 19 | 0.952 | 0.052 | 1.65E+00 | 1.42E−185 | 2.52E−178 |
| Lgals3 | 3 | 19 | 0.938 | 0.076 | 1.63E+00 | 3.13E−161 | 5.54E−154 |
| Id2 | 3 | 20 | 0.824 | 0.059 | 1.41E+00 | 1.24E−160 | 2.19E−153 |
| Cxcr6 | 3 | 22 | 0.935 | 0.086 | 1.54E+00 | 2.08E−206 | 3.69E−199 |
| Cxcr6 | 3 | 24 | 0.935 | 0.049 | 1.56E+00 | 5.53E−210 | 9.78E−203 |
| Sepp1 | 3 | 24 | 0.904 | 0.09 | 1.35E+00 | 6.20E−174 | 1.10E−166 |
| Gimap3 | 3 | 24 | 0.88 | 0.049 | 1.21E+00 | 3.21E−165 | 5.69E−158 |
| Cxcr6 | 3 | 25 | 0.935 | 0.054 | 1.56E+00 | 1.35E−189 | 2.40E−182 |
| Lgals3 | 3 | 25 | 0.938 | 0.086 | 1.65E+00 | 1.46E−189 | 2.59E−182 |
| Car2 | 3 | 25 | 0.915 | 0.054 | 1.49E+00 | 4.23E−172 | 7.49E−165 |
| Il7r | 3 | 25 | 0.911 | 0.081 | 1.40E+00 | 2.80E−161 | 4.96E−154 |
| Emb | 3 | 25 | 0.914 | 0.086 | 1.32E+00 | 9.93E−160 | 1.76E−152 |
| Tmem176b | 3 | 26 | 0.975 | 0.101 | 1.75E+00 | 6.96E−260 | 1.23E−252 |
| Cxcr6 | 3 | 26 | 0.935 | 0.055 | 1.54E+00 | 1.21E−199 | 2.15E−192 |
| Tmem176b | 3 | 29 | 0.975 | 0.047 | 1.81E+00 | 9.29E−181 | 1.64E−173 |
| Tmem176a | 3 | 29 | 0.952 | 0.034 | 1.67E+00 | 5.29E−161 | 9.37E−154 |
| Tmem176a | 4 | 5 | 0.979 | 0.018 | 1.97E+00 | 0.00E+00 | 0.00E+00 |
| Tmem176b | 4 | 5 | 0.976 | 0.016 | 1.95E+00 | 0.00E+00 | 0.00E+00 |
| S100a4 | 4 | 5 | 0.884 | 0.006 | 1.83E+00 | 0.00E+00 | 0.00E+00 |
| Fcer1g | 4 | 5 | 0.98 | 0.02 | 1.82E+00 | 0.00E+00 | 0.00E+00 |
| Gem | 4 | 5 | 0.777 | 0.028 | 1.72E+00 | 0.00E+00 | 0.00E+00 |
| Nr4a1 | 4 | 5 | 0.774 | 0.095 | 1.43E+00 | 0.00E+00 | 0.00E+00 |
| Ckb | 4 | 5 | 0.891 | 0.022 | 1.42E+00 | 0.00E+00 | 0.00E+00 |
| Fosb | 4 | 5 | 0.623 | 0.011 | 1.32E+00 | 0.00E+00 | 0.00E+00 |
| Cd83 | 4 | 5 | 0.578 | 0.037 | 1.27E+00 | 0.00E+00 | 0.00E+00 |
| Lgals3 | 4 | 5 | 0.742 | 0.012 | 1.25E+00 | 0.00E+00 | 0.00E+00 |
| Ncoa7 | 4 | 5 | 0.798 | 0.048 | 1.22E+00 | 0.00E+00 | 0.00E+00 |
| Gadd45b | 4 | 5 | 0.623 | 0.051 | 1.22E+00 | 0.00E+00 | 0.00E+00 |
| Lmo4 | 4 | 5 | 0.817 | 0.101 | 1.17E+00 | 0.00E+00 | 0.00E+00 |
| Prr29 | 4 | 5 | 0.784 | 0.002 | 1.15E+00 | 0.00E+00 | 0.00E+00 |
| Car2 | 4 | 5 | 0.803 | 0.041 | 1.15E+00 | 0.00E+00 | 0.00E+00 |
| Sdc4 | 4 | 5 | 0.748 | 0.004 | 1.12E+00 | 0.00E+00 | 0.00E+00 |
| Bhlhe40 | 4 | 5 | 0.709 | 0.015 | 1.11E+00 | 0.00E+00 | 0.00E+00 |
| Rasl11a | 4 | 5 | 0.603 | 0.002 | 1.10E+00 | 0.00E+00 | 0.00E+00 |
| Rora | 4 | 5 | 0.73 | 0.016 | 1.03E+00 | 0.00E+00 | 0.00E+00 |
| Chad | 4 | 5 | 0.724 | 0.003 | 9.89E−01 | 0.00E+00 | 0.00E+00 |
| Cited4 | 4 | 5 | 0.614 | 0.003 | 9.82E−01 | 0.00E+00 | 0.00E+00 |
| Nrgn | 4 | 5 | 0.719 | 0.004 | 9.65E−01 | 0.00E+00 | 0.00E+00 |
| Maff | 4 | 5 | 0.631 | 0.023 | 9.24E−01 | 0.00E+00 | 0.00E+00 |
| Cd81 | 4 | 5 | 0.65 | 0.035 | 9.17E−01 | 0.00E+00 | 0.00E+00 |
| Dhrs3 | 4 | 5 | 0.674 | 0.009 | 8.22E−01 | 0.00E+00 | 0.00E+00 |
| Serpinb1a | 4 | 5 | 0.606 | 0.01 | 8.13E−01 | 0.00E+00 | 0.00E+00 |
| Fam110a | 4 | 5 | 0.55 | 0.011 | 8.09E−01 | 0.00E+00 | 0.00E+00 |
| Ffar2 | 4 | 5 | 0.563 | 0.002 | 7.72E−01 | 0.00E+00 | 0.00E+00 |
| Ccr6 | 4 | 5 | 0.618 | 0.003 | 7.34E−01 | 0.00E+00 | 0.00E+00 |
| Espn | 4 | 5 | 0.591 | 0.008 | 7.30E−01 | 0.00E+00 | 0.00E+00 |
| Rorc | 4 | 5 | 0.557 | 0.003 | 6.76E−01 | 0.00E+00 | 0.00E+00 |
| Kit | 4 | 5 | 0.551 | 0.005 | 6.47E−01 | 0.00E+00 | 0.00E+00 |
| Serpina3g | 4 | 5 | 0.543 | 0.005 | 8.53E−01 | 1.9E−313 | 3.38E−306 |
| Upp1 | 4 | 5 | 0.507 | 0.001 | 7.04E−01 | 3.2E−313 | 5.80E−306 |
| Tnfrsf25 | 4 | 5 | 0.607 | 0.034 | 6.52E−01 | 7.1E−313 | 1.27E−305 |
| Hebp1 | 4 | 5 | 0.565 | 0.013 | 8.70E−01 | 0.00E+00 | 3.61E−302 |
| S100a6 | 4 | 5 | 0.553 | 0.019 | 1.02E+00 | 1.72E−294 | 3.05E−287 |
| St6galnac3 | 4 | 5 | 0.529 | 0.022 | 5.66E−01 | 3.73E−294 | 6.61E−287 |
| Podnl1 | 4 | 5 | 0.606 | 0.042 | 6.73E−01 | 3.90E−287 | 6.91E−280 |
| Asb2 | 4 | 5 | 0.504 | 0.006 | 6.39E−01 | 7.62E−284 | 1.35E−276 |
| Igfbp7 | 4 | 5 | 0.515 | 0.005 | 7.54E−01 | 9.67E−280 | 1.71E−272 |
| Ube2e3 | 4 | 5 | 0.589 | 0.049 | 6.87E−01 | 2.18E−273 | 3.87E−266 |
| Ptms | 4 | 5 | 0.618 | 0.05 | 7.02E−01 | 8.80E−265 | 1.56E−257 |
| Acot7 | 4 | 5 | 0.573 | 0.037 | 6.36E−01 | 6.45E−254 | 1.14E−246 |
| Dgat1 | 4 | 5 | 0.504 | 0.025 | 6.34E−01 | 1.02E−248 | 1.81E−241 |
| Egr1 | 4 | 5 | 0.526 | 0.055 | 9.05E−01 | 2.39E−244 | 4.23E−237 |
| Il18r1 | 4 | 5 | 0.548 | 0.065 | 5.74E−01 | 8.58E−226 | 1.52E−218 |
| Samsn1 | 4 | 5 | 0.543 | 0.06 | 5.85E−01 | 1.32E−212 | 2.34E−205 |
| Cmc2 | 4 | 5 | 0.562 | 0.1 | 5.33E−01 | 6.39E−176 | 1.13E−168 |
| S100a4 | 4 | 6 | 0.884 | 0.085 | 1.74E+00 | 0.00E+00 | 0.00E+00 |
| Id2 | 4 | 6 | 0.854 | 0.096 | 1.56E+00 | 0.00E+00 | 0.00E+00 |
| Cxcr6 | 4 | 6 | 0.861 | 0.078 | 1.26E+00 | 0.00E+00 | 0.00E+00 |
| Prr29 | 4 | 6 | 0.784 | 0.02 | 1.13E+00 | 0.00E+00 | 0.00E+00 |
| Rasl11a | 4 | 6 | 0.603 | 0.023 | 1.08E+00 | 0.00E+00 | 0.00E+00 |
| Car2 | 4 | 6 | 0.803 | 0.101 | 1.07E+00 | 0.00E+00 | 0.00E+00 |
| Rora | 4 | 6 | 0.73 | 0.09 | 9.45E−01 | 0.00E+00 | 0.00E+00 |
| Cited4 | 4 | 6 | 0.614 | 0.043 | 9.36E−01 | 0.00E+00 | 0.00E+00 |
| Chad | 4 | 6 | 0.724 | 0.051 | 9.35E−01 | 0.00E+00 | 0.00E+00 |
| Nrgn | 4 | 6 | 0.719 | 0.04 | 9.26E−01 | 0.00E+00 | 0.00E+00 |
| Cd81 | 4 | 6 | 0.65 | 0.031 | 9.17E−01 | 0.00E+00 | 0.00E+00 |
| Dhrs3 | 4 | 6 | 0.674 | 0.04 | 7.90E−01 | 0.00E+00 | 0.00E+00 |
| Espn | 4 | 6 | 0.591 | 0.025 | 7.13E−01 | 0.00E+00 | 0.00E+00 |
| Igfbp7 | 4 | 6 | 0.515 | 0.053 | 7.06E−01 | 0.00E+00 | 0.00E+00 |
| Podnl1 | 4 | 6 | 0.606 | 0.047 | 6.67E−01 | 0.00E+00 | 0.00E+00 |
| Tnfrsf25 | 4 | 6 | 0.607 | 0.027 | 6.58E−01 | 0.00E+00 | 0.00E+00 |
| Acot7 | 4 | 6 | 0.573 | 0.062 | 6.12E−01 | 0.00E+00 | 0.00E+00 |
| Serpinb1a | 4 | 6 | 0.606 | 0.062 | 7.56E−01 | 2.3E−315 | 4.18E−308 |
| Rorc | 4 | 6 | 0.557 | 0.034 | 6.43E−01 | l.OE−313 | 1.85E−306 |
| Tcf7 | 4 | 6 | 0.617 | 0.058 | 6.93E−01 | 1.1E−311 | 2.00E−304 |
| Serpina3g | 4 | 6 | 0.543 | 0.064 | 7.94E−01 | 1.48E−301 | 2.62E−294 |
| Upp1 | 4 | 6 | 0.507 | 0.044 | 6.57E−01 | 1.60E−284 | 2.84E−277 |
| Asb2 | 4 | 6 | 0.504 | 0.05 | 5.92E−01 | 1.76E−279 | 3.11E−272 |
| Maff | 4 | 6 | 0.631 | 0.072 | 8.70E−01 | 2.21E−273 | 3.91E−266 |
| St6galnac3 | 4 | 6 | 0.529 | 0.033 | 5.53E−01 | 3.37E−264 | 5.97E−257 |
| Trbc2 | 4 | 6 | 0.544 | 0.084 | 9.70E−01 | 2.49E−262 | 4.40E−255 |
| Il18r1 | 4 | 6 | 0.548 | 0.048 | 5.90E−01 | 2.56E−251 | 4.53E−244 |
| Txk | 4 | 6 | 0.564 | 0.076 | 6.13E−01 | 4.15E−247 | 7.36E−240 |
| Kit | 4 | 6 | 0.551 | 0.082 | 5.65E−01 | 5.62E−233 | 9.96E−226 |
| Tmem176a | 4 | 7 | 0.979 | 0.021 | 1.96E+00 | 0.00E+00 | 0.00E+00 |
| Tmem176b | 4 | 7 | 0.976 | 0.031 | 1.92E+00 | 0.00E+00 | 0.00E+00 |
| Fcer1g | 4 | 7 | 0.98 | 0.019 | 1.82E+00 | 0.00E+00 | 0.00E+00 |
| S100a4 | 4 | 7 | 0.884 | 0.063 | 1.68E+00 | 0.00E+00 | 0.00E+00 |
| Gem | 4 | 7 | 0.777 | 0.088 | 1.64E+00 | 0.00E+00 | 0.00E+00 |
| Fosb | 4 | 7 | 0.623 | 0.023 | 1.31E+00 | 0.00E+00 | 0.00E+00 |
| Lgals3 | 4 | 7 | 0.742 | 0.03 | 1.21E+00 | 0.00E+00 | 0.00E+00 |
| Cxcr6 | 4 | 7 | 0.861 | 0.099 | 1.20E+00 | 0.00E+00 | 0.00E+00 |
| Car2 | 4 | 7 | 0.803 | 0.019 | 1.16E+00 | 0.00E+00 | 0.00E+00 |
| Prr29 | 4 | 7 | 0.784 | 0.001 | 1.15E+00 | 0.00E+00 | 0.00E+00 |
| Sdc4 | 4 | 7 | 0.748 | 0.041 | 1.08E+00 | 0.00E+00 | 0.00E+00 |
| Rasl11a | 4 | 7 | 0.603 | 0.028 | 1.07E+00 | 0.00E+00 | 0.00E+00 |
| Chad | 4 | 7 | 0.724 | 0.005 | 9.87E−01 | 0.00E+00 | 0.00E+00 |
| Cited4 | 4 | 7 | 0.614 | 0.006 | 9.76E−01 | 0.00E+00 | 0.00E+00 |
| Maff | 4 | 7 | 0.631 | 0.052 | 8.78E−01 | 0.00E+00 | 0.00E+00 |
| Nrgn | 4 | 7 | 0.719 | 0.042 | 8.74E−01 | 0.00E+00 | 0.00E+00 |
| Hebp1 | 4 | 7 | 0.565 | 0.016 | 8.67E−01 | 0.00E+00 | 0.00E+00 |
| Serpinb1a | 4 | 7 | 0.606 | 0.022 | 7.97E−01 | 0.00E+00 | 0.00E+00 |
| Serpina3g | 4 | 7 | 0.543 | 0.045 | 7.83E−01 | 0.00E+00 | 0.00E+00 |
| Fam110a | 4 | 7 | 0.55 | 0.049 | 7.65E−01 | 0.00E+00 | 0.00E+00 |
| Ffar2 | 4 | 7 | 0.563 | 0.014 | 7.54E−01 | 0.00E+00 | 0.00E+00 |
| Igfbp7 | 4 | 7 | 0.515 | 0.01 | 7.45E−01 | 0.00E+00 | 0.00E+00 |
| Upp1 | 4 | 7 | 0.507 | 0.004 | 7.01E−01 | 0.00E+00 | 0.00E+00 |
| Espn | 4 | 7 | 0.591 | 0.043 | 6.93E−01 | 0.00E+00 | 0.00E+00 |
| Ccr6 | 4 | 7 | 0.618 | 0.052 | 6.75E−01 | 0.00E+00 | 0.00E+00 |
| Rorc | 4 | 7 | 0.557 | 0.019 | 6.58E−01 | 0.00E+00 | 0.00E+00 |
| Kit | 4 | 7 | 0.551 | 0.006 | 6.44E−01 | 0.00E+00 | 0.00E+00 |
| Il18r1 | 4 | 7 | 0.548 | 0.034 | 6.04E−01 | 0.00E+00 | 0.00E+00 |
| Ube2e3 | 4 | 7 | 0.589 | 0.094 | 6.32E−01 | 8.0E−313 | 1.42E−305 |
| Dgat1 | 4 | 7 | 0.504 | 0.063 | 5.90E−01 | 3.12E−273 | 5.52E−266 |
| Runx3 | 4 | 7 | 0.508 | 0.101 | 4.70E−01 | 6.20E−216 | 1.10E−208 |
| S100a4 | 4 | 8 | 0.884 | 0.101 | 1.65E+00 | 0.00E+00 | 0.00E+00 |
| Cxcr6 | 4 | 8 | 0.861 | 0.085 | 1.26E+00 | 0.00E+00 | 0.00E+00 |
| Ncoa7 | 4 | 8 | 0.798 | 0.08 | 1.18E+00 | 0.00E+00 | 0.00E+00 |
| Prr29 | 4 | 8 | 0.784 | 0.008 | 1.14E+00 | 0.00E+00 | 0.00E+00 |
| Rasl11a | 4 | 8 | 0.603 | 0.019 | 1.08E+00 | 0.00E+00 | 0.00E+00 |
| Chad | 4 | 8 | 0.724 | 0.042 | 9.48E−01 | 0.00E+00 | 0.00E+00 |
| Cd81 | 4 | 8 | 0.65 | 0.027 | 9.21E−01 | 0.00E+00 | 0.00E+00 |
| Hebp1 | 4 | 8 | 0.565 | 0.025 | 8.58E−01 | 0.00E+00 | 0.00E+00 |
| Ffar2 | 4 | 8 | 0.563 | 0.021 | 7.50E−01 | 0.00E+00 | 0.00E+00 |
| Serpinb1a | 4 | 8 | 0.606 | 0.052 | 7.43E−01 | 0.00E+00 | 0.00E+00 |
| Ccr6 | 4 | 8 | 0.618 | 0.019 | 7.17E−01 | 0.00E+00 | 0.00E+00 |
| Espn | 4 | 8 | 0.591 | 0.027 | 7.10E−01 | 0.00E+00 | 0.00E+00 |
| Rorc | 4 | 8 | 0.557 | 0.025 | 6.54E−01 | 0.00E+00 | 0.00E+00 |
| Tnfrsf25 | 4 | 8 | 0.607 | 0.048 | 6.34E−01 | 0.00E+00 | 0.00E+00 |
| Fosb | 4 | 8 | 0.623 | 0.091 | 1.18E+00 | 2.12E−305 | 3.76E−298 |
| Upp1 | 4 | 8 | 0.507 | 0.026 | 6.78E−01 | 5.62E−296 | 9.96E−289 |
| Igfbp7 | 4 | 8 | 0.515 | 0.032 | 7.20E−01 | 2.28E−288 | 4.04E−281 |
| Fam110a | 4 | 8 | 0.55 | 0.053 | 7.49E−01 | 4.44E−285 | 7.86E−278 |
| Anxa5 | 4 | 8 | 0.609 | 0.088 | 6.17E−01 | 4.30E−283 | 7.62E−276 |
| Rgs10 | 4 | 8 | 0.564 | 0.099 | 5.69E−01 | 2.60E−228 | 4.60E−221 |
| Asb2 | 4 | 8 | 0.504 | 0.088 | 5.43E−01 | 1.25E−194 | 2.21E−187 |
| Tmem176a | 4 | 9 | 0.979 | 0.034 | 1.95E+00 | 0.00E+00 | 0.00E+00 |
| Tmem176b | 4 | 9 | 0.976 | 0.039 | 1.92E+00 | 0.00E+00 | 0.00E+00 |
| Cd7 | 4 | 9 | 0.876 | 0.077 | 1.81E+00 | 0.00E+00 | 0.00E+00 |
| S100a4 | 4 | 9 | 0.884 | 0.02 | 1.81E+00 | 0.00E+00 | 0.00E+00 |
| Fcer1g | 4 | 9 | 0.98 | 0.038 | 1.80E+00 | 0.00E+00 | 0.00E+00 |
| Il7r | 4 | 9 | 0.951 | 0.043 | 1.59E+00 | 0.00E+00 | 0.00E+00 |
| Ckb | 4 | 9 | 0.891 | 0.052 | 1.38E+00 | 0.00E+00 | 0.00E+00 |
| Cxcr6 | 4 | 9 | 0.861 | 0.017 | 1.34E+00 | 0.00E+00 | 0.00E+00 |
| Bcl2 | 4 | 9 | 0.85 | 0.095 | 1.24E+00 | 0.00E+00 | 0.00E+00 |
| Lgals3 | 4 | 9 | 0.742 | 0.035 | 1.21E+00 | 0.00E+00 | 0.00E+00 |
| Prr29 | 4 | 9 | 0.784 | 0.008 | 1.14E+00 | 0.00E+00 | 0.00E+00 |
| S100a10 | 4 | 9 | 0.824 | 0.093 | 1.14E+00 | 0.00E+00 | 0.00E+00 |
| Ramp1 | 4 | 9 | 0.719 | 0.053 | 1.04E+00 | 6.49E−304 | 1.15E−296 |
| Sdc4 | 4 | 9 | 0.748 | 0.038 | 1.07E+00 | 1.97E−283 | 3.48E−276 |
| Chad | 4 | 9 | 0.724 | 0.009 | 9.83E−01 | 5.16E−265 | 9.14E−258 |
| Bst2 | 4 | 9 | 0.687 | 0.099 | 7.89E−01 | 6.89E−259 | 1.22E−251 |
| Cited4 | 4 | 9 | 0.614 | 0.009 | 9.72E−01 | 8.64E−251 | 1.53E−243 |
| Rora | 4 | 9 | 0.73 | 0.013 | 1.04E+00 | 4.19E−243 | 7.43E−236 |
| Sla | 4 | 9 | 0.697 | 0.063 | 1.01E+00 | 1.41E−239 | 2.50E−232 |
| Igfbp7 | 4 | 9 | 0.515 | 0.01 | 7.49E−01 | 1.42E−239 | 2.52E−232 |
| S100a6 | 4 | 9 | 0.553 | 0.029 | 1.00E+00 | 1.52E−235 | 2.69E−228 |
| Gpr183 | 4 | 9 | 0.675 | 0.07 | 8.23E−01 | 1.14E−234 | 2.02E−227 |
| Hebp1 | 4 | 9 | 0.565 | 0.024 | 8.60E−01 | 9.49E−234 | 1.68E−226 |
| Podnl1 | 4 | 9 | 0.606 | 0.016 | 7.01E−01 | 5.77E−230 | 1.02E−222 |
| Tnfrsf25 | 4 | 9 | 0.607 | 0.012 | 6.74E−01 | 5.55E−228 | 9.83E−221 |
| Bhlhe40 | 4 | 9 | 0.709 | 0.05 | 1.06E+00 | 4.15E−226 | 7.35E−219 |
| Ffar2 | 4 | 9 | 0.563 | 0.014 | 7.57E−01 | 5.97E−226 | 1.06E−218 |
| Serpina3g | 4 | 9 | 0.543 | 0.021 | 8.35E−01 | 8.93E−226 | 1.58E−218 |
| Rorc | 4 | 9 | 0.557 | 0.006 | 6.71E−01 | 2.53E−224 | 4.48E−217 |
| Rasl11a | 4 | 9 | 0.603 | 0.004 | 1.10E+00 | 7.17E−221 | 1.27E−213 |
| Ccr6 | 4 | 9 | 0.618 | 0.015 | 7.19E−01 | 6.70E−205 | 1.19E−197 |
| Tcf7 | 4 | 9 | 0.617 | 0.052 | 6.98E−01 | 1.71E−203 | 3.03E−196 |
| Anxa5 | 4 | 9 | 0.609 | 0.077 | 6.29E−01 | 5.35E−203 | 9.46E−196 |
| Gpr132 | 4 | 9 | 0.66 | 0.038 | 8.02E−01 | 1.11E−200 | 1.96E−193 |
| Asb2 | 4 | 9 | 0.504 | 0.025 | 6.13E−01 | 1.60E−197 | 2.83E−190 |
| Espn | 4 | 9 | 0.591 | 0.023 | 7.16E−01 | 3.83E−197 | 6.79E−190 |
| Serpinb1a | 4 | 9 | 0.606 | 0.067 | 7.16E−01 | 2.30E−194 | 4.08E−187 |
| Upp1 | 4 | 9 | 0.507 | 0.015 | 6.89E−01 | 2.77E−191 | 4.90E−184 |
| Maff | 4 | 9 | 0.631 | 0.015 | 9.33E−01 | 8.58E−187 | 1.52E−179 |
| Il18r1 | 4 | 9 | 0.548 | 0.009 | 6.35E−01 | 1.22E−178 | 2.17E−171 |
| Kit | 4 | 9 | 0.551 | 0.007 | 6.43E−01 | 1.46E−175 | 2.58E−168 |
| Runx3 | 4 | 9 | 0.508 | 0.097 | 4.57E−01 | 5.54E−169 | 9.81E−162 |
| Dgat1 | 4 | 9 | 0.504 | 0.042 | 6.19E−01 | 2.01E−167 | 3.56E−160 |
| St6galnac3 | 4 | 9 | 0.529 | 0.019 | 5.69E−01 | 8.27E−160 | 1.46E−152 |
| Txk | 4 | 9 | 0.564 | 0.055 | 6.34E−01 | 4.75E−159 | 8.41E−152 |
| Tmem176a | 4 | 10 | 0.979 | 0.023 | 1.96E+00 | 0.00E+00 | 0.00E+00 |
| Tmem176b | 4 | 10 | 0.976 | 0.025 | 1.92E+00 | 0.00E+00 | 0.00E+00 |
| Cd7 | 4 | 10 | 0.876 | 0.043 | 1.84E+00 | 0.00E+00 | 0.00E+00 |
| S100a4 | 4 | 10 | 0.884 | 0.014 | 1.83E+00 | 0.00E+00 | 0.00E+00 |
| Fcer1g | 4 | 10 | 0.98 | 0.032 | 1.80E+00 | 0.00E+00 | 0.00E+00 |
| Id2 | 4 | 10 | 0.854 | 0.051 | 1.61E+00 | 0.00E+00 | 0.00E+00 |
| Il7r | 4 | 10 | 0.951 | 0.026 | 1.60E+00 | 0.00E+00 | 0.00E+00 |
| Sepp1 | 4 | 10 | 0.944 | 0.075 | 1.42E+00 | 0.00E+00 | 0.00E+00 |
| Ckb | 4 | 10 | 0.891 | 0.027 | 1.42E+00 | 0.00E+00 | 0.00E+00 |
| Nr4a1 | 4 | 10 | 0.774 | 0.093 | 1.41E+00 | 0.00E+00 | 0.00E+00 |
| Cxcr6 | 4 | 10 | 0.861 | 0.013 | 1.34E+00 | 0.00E+00 | 0.00E+00 |
| Bcl2 | 4 | 10 | 0.85 | 0.05 | 1.30E+00 | 0.00E+00 | 0.00E+00 |
| Fosb | 4 | 10 | 0.623 | 0.041 | 1.28E+00 | 0.00E+00 | 0.00E+00 |
| Lgals3 | 4 | 10 | 0.742 | 0.03 | 1.22E+00 | 0.00E+00 | 0.00E+00 |
| S100a10 | 4 | 10 | 0.824 | 0.053 | 1.20E+00 | 0.00E+00 | 0.00E+00 |
| Ncoa7 | 4 | 10 | 0.798 | 0.074 | 1.19E+00 | 0.00E+00 | 0.00E+00 |
| Emb | 4 | 10 | 0.846 | 0.057 | 1.17E+00 | 0.00E+00 | 0.00E+00 |
| Prr29 | 4 | 10 | 0.784 | 0.007 | 1.14E+00 | 0.00E+00 | 0.00E+00 |
| Rasl11a | 4 | 10 | 0.603 | 0.005 | 1.10E+00 | 0.00E+00 | 0.00E+00 |
| Ramp1 | 4 | 10 | 0.719 | 0.019 | 1.08E+00 | 0.00E+00 | 0.00E+00 |
| Car2 | 4 | 10 | 0.803 | 0.1 | 1.08E+00 | 0.00E+00 | 0.00E+00 |
| Bhlhe40 | 4 | 10 | 0.709 | 0.04 | 1.07E+00 | 0.00E+00 | 0.00E+00 |
| Sdc4 | 4 | 10 | 0.748 | 0.044 | 1.05E+00 | 0.00E+00 | 0.00E+00 |
| Rora | 4 | 10 | 0.73 | 0.008 | 1.04E+00 | 0.00E+00 | 0.00E+00 |
| S100a6 | 4 | 10 | 0.553 | 0.01 | 1.03E+00 | 0.00E+00 | 0.00E+00 |
| Sla | 4 | 10 | 0.697 | 0.05 | 1.02E+00 | 0.00E+00 | 0.00E+00 |
| Chad | 4 | 10 | 0.724 | 0.005 | 9.84E−01 | 0.00E+00 | 0.00E+00 |
| Cited4 | 4 | 10 | 0.614 | 0.006 | 9.76E−01 | 0.00E+00 | 0.00E+00 |
| Maff | 4 | 10 | 0.631 | 0.009 | 9.38E−01 | 0.00E+00 | 0.00E+00 |
| Hebp1 | 4 | 10 | 0.565 | 0.016 | 8.67E−01 | 0.00E+00 | 0.00E+00 |
| Gpr183 | 4 | 10 | 0.675 | 0.048 | 8.46E−01 | 0.00E+00 | 0.00E+00 |
| Serpina3g | 4 | 10 | 0.543 | 0.014 | 8.44E−01 | 0.00E+00 | 0.00E+00 |
| Bst2 | 4 | 10 | 0.687 | 0.051 | 8.44E−01 | 0.00E+00 | 0.00E+00 |
| Selplg | 4 | 10 | 0.712 | 0.09 | 8.21E−01 | 0.00E+00 | 0.00E+00 |
| Gpr132 | 4 | 10 | 0.66 | 0.025 | 8.18E−01 | 0.00E+00 | 0.00E+00 |
| Ffar2 | 4 | 10 | 0.563 | 0.009 | 7.63E−01 | 0.00E+00 | 0.00E+00 |
| Igfbp7 | 4 | 10 | 0.515 | 0.012 | 7.47E−01 | 0.00E+00 | 0.00E+00 |
| Tcf7 | 4 | 10 | 0.617 | 0.029 | 7.29E−01 | 0.00E+00 | 0.00E+00 |
| Ccr6 | 4 | 10 | 0.618 | 0.014 | 7.20E−01 | 0.00E+00 | 0.00E+00 |
| Podnl1 | 4 | 10 | 0.606 | 0.008 | 7.08E−01 | 0.00E+00 | 0.00E+00 |
| Espn | 4 | 10 | 0.591 | 0.029 | 7.08E−01 | 0.00E+00 | 0.00E+00 |
| Upp1 | 4 | 10 | 0.507 | 0.009 | 6.97E−01 | 0.00E+00 | 0.00E+00 |
| Tnfrsf25 | 4 | 10 | 0.607 | 0.011 | 6.76E−01 | 0.00E+00 | 0.00E+00 |
| Rorc | 4 | 10 | 0.557 | 0.002 | 6.76E−01 | 0.00E+00 | 0.00E+00 |
| Znrf1 | 4 | 10 | 0.632 | 0.057 | 6.75E−01 | 0.00E+00 | 0.00E+00 |
| Anxa5 | 4 | 10 | 0.609 | 0.043 | 6.67E−01 | 0.00E+00 | 0.00E+00 |
| Txk | 4 | 10 | 0.564 | 0.03 | 6.66E−01 | 0.00E+00 | 0.00E+00 |
| Kit | 4 | 10 | 0.551 | 0.007 | 6.44E−01 | 0.00E+00 | 0.00E+00 |
| Il18r1 | 4 | 10 | 0.548 | 0.005 | 6.38E−01 | 0.00E+00 | 0.00E+00 |
| Asb2 | 4 | 10 | 0.504 | 0.01 | 6.34E−01 | 0.00E+00 | 0.00E+00 |
| St6galnac3 | 4 | 10 | 0.529 | 0.019 | 5.68E−01 | 0.00E+00 | 0.00E+00 |
| Acot7 | 4 | 10 | 0.573 | 0.099 | 5.72E−01 | 2.1E−316 | 0.00E+00 |
| Serpinb1a | 4 | 10 | 0.606 | 0.088 | 6.82E−01 | 3.77E−307 | 6.68E−300 |
| S100a11 | 4 | 10 | 0.563 | 0.09 | 6.10E−01 | 3.58E−302 | 6.34E−295 |
| Tnfaip3 | 4 | 10 | 0.573 | 0.07 | 7.74E−01 | 1.16E−298 | 2.05E−291 |
| Vps37b | 4 | 10 | 0.549 | 0.074 | 9.19E−01 | 2.39E−275 | 4.23E−268 |
| Runx3 | 4 | 10 | 0.508 | 0.049 | 5.35E−01 | 2.40E−275 | 4.25E−268 |
| Hcst | 4 | 10 | 0.511 | 0.089 | 5.47E−01 | 8.65E−248 | 1.53E−240 |
| Prr29 | 4 | 11 | 0.784 | 0.026 | 1.11E+00 | 0.00E+00 | 0.00E+00 |
| Cd81 | 4 | 11 | 0.65 | 0.042 | 8.99E−01 | 3.13E−256 | 5.54E−249 |
| Ccr6 | 4 | 11 | 0.618 | 0.032 | 7.01E−01 | 2.96E−246 | 5.24E−239 |
| Rasl11a | 4 | 11 | 0.603 | 0.038 | 1.06E+00 | 1.56E−229 | 2.77E−222 |
| Hebp1 | 4 | 11 | 0.565 | 0.044 | 8.35E−01 | 1.32E−195 | 2.33E−188 |
| Rorc | 4 | 11 | 0.557 | 0.052 | 6.21E−01 | 1.54E−181 | 2.73E−174 |
| Igfbp7 | 4 | 11 | 0.515 | 0.041 | 7.13E−01 | 2.65E−166 | 4.69E−159 |
| Ncoa7 | 4 | 12 | 0.798 | 0.096 | 1.16E+00 | 0.00E+00 | 0.00E+00 |
| Prr29 | 4 | 12 | 0.784 | 0.054 | 1.09E+00 | 0.00E+00 | 0.00E+00 |
| Chad | 4 | 12 | 0.724 | 0.072 | 9.18E−01 | 8.94E−278 | 1.58E−270 |
| Sdc4 | 4 | 12 | 0.748 | 0.095 | 1.02E+00 | 4.64E−275 | 8.21E−268 |
| Ccr6 | 4 | 12 | 0.618 | 0.02 | 7.15E−01 | 6.12E−252 | 1.08E−244 |
| Rasl11a | 4 | 12 | 0.603 | 0.041 | 1.04E+00 | 1.99E−216 | 3.52E−209 |
| Hebp1 | 4 | 12 | 0.565 | 0.042 | 8.35E−01 | 8.58E−194 | 1.52E−186 |
| Rorc | 4 | 12 | 0.557 | 0.044 | 6.31E−01 | 2.96E−186 | 5.24E−179 |
| Upp1 | 4 | 12 | 0.507 | 0.039 | 6.61E−01 | 1.53E−164 | 2.71E−157 |
| Igfbp7 | 4 | 12 | 0.515 | 0.045 | 6.86E−01 | 4.77E−160 | 8.44E−153 |
| Cxcr6 | 4 | 14 | 0.861 | 0.091 | 1.22E+00 | 6.27E−239 | 1.11E−231 |
| Prr29 | 4 | 14 | 0.784 | 0.063 | 9.79E−01 | 1.67E−222 | 2.95E−215 |
| Chad | 4 | 14 | 0.724 | 0.078 | 9.04E−01 | 9.40E−171 | 1.66E−163 |
| Tmem176a | 4 | 17 | 0.979 | 0.059 | 1.91E+00 | 0.00E+00 | 0.00E+00 |
| Tmem176b | 4 | 17 | 0.976 | 0.094 | 1.83E+00 | 0.00E+00 | 0.00E+00 |
| S100a4 | 4 | 17 | 0.884 | 0.012 | 1.83E+00 | 9.45E−277 | 1.67E−269 |
| Prr29 | 4 | 17 | 0.784 | 0.006 | 1.14E+00 | 2.32E−207 | 4.11E−200 |
| Sdc4 | 4 | 17 | 0.748 | 0.023 | 1.10E+00 | 1.01E−170 | 1.78E−163 |
| Ncoa7 | 4 | 17 | 0.798 | 0.094 | 1.16E+00 | 2.00E−162 | 3.54E−155 |
| Ramp1 | 4 | 17 | 0.719 | 0.032 | 1.05E+00 | 2.36E−159 | 4.18E−152 |
| Tmem176a | 4 | 18 | 0.979 | 0.011 | 1.98E+00 | 0.00E+00 | 0.00E+00 |
| S100a4 | 4 | 18 | 0.884 | 0.028 | 1.79E+00 | 9.91E−215 | 1.75E−207 |
| Cd7 | 4 | 18 | 0.876 | 0.05 | 1.84E+00 | 2.91E−195 | 5.15E−188 |
| Ckb | 4 | 18 | 0.891 | 0.1 | 1.34E+00 | 1.20E−194 | 2.13E−187 |
| Cxcr6 | 4 | 18 | 0.861 | 0.06 | 1.26E+00 | 2.34E−179 | 4.14E−172 |
| Prr29 | 4 | 18 | 0.784 | 0.004 | 1.15E+00 | 9.47E−171 | 1.68E−163 |
| Car2 | 4 | 18 | 0.803 | 0.028 | 1.16E+00 | 2.62E−160 | 4.64E−153 |
| Tmem176a | 4 | 19 | 0.979 | 0.052 | 1.93E+00 | 1.02E−193 | 1.81E−186 |
| Tmem176b | 4 | 19 | 0.976 | 0.096 | 1.87E+00 | 2.33E−175 | 4.12E−168 |
| Id2 | 4 | 20 | 0.854 | 0.059 | 1.47E+00 | 1.98E−183 | 3.50E−176 |
| Prr29 | 4 | 20 | 0.784 | 0.025 | 1.13E+00 | 1.18E−165 | 2.09E−158 |
| Prr29 | 4 | 22 | 0.784 | 0.022 | 1.12E+00 | 1.40E−168 | 2.48E−161 |
| Cxcr6 | 4 | 22 | 0.861 | 0.086 | 1.24E+00 | 3.90E−164 | 6.90E−157 |
| Sepp1 | 4 | 24 | 0.944 | 0.09 | 1.41E+00 | 5.72E−219 | 1.01E−211 |
| Cxcr6 | 4 | 24 | 0.861 | 0.049 | 1.26E+00 | 1.18E−169 | 2.09E−162 |
| Bcl2 | 4 | 24 | 0.85 | 0.094 | 1.15E+00 | 9.74E−163 | 1.72E−155 |
| Fcer1g | 4 | 25 | 0.98 | 0.086 | 1.71E+00 | 1.44E−228 | 2.56E−221 |
| Il7r | 4 | 25 | 0.951 | 0.081 | 1.51E+00 | 2.87E−186 | 5.07E−179 |
| Tmem176b | 4 | 26 | 0.976 | 0.101 | 1.84E+00 | 1.93E−273 | 3.41E−266 |
| S100a4 | 4 | 26 | 0.884 | 0.051 | 1.78E+00 | 6.55E−191 | 1.16E−183 |
| Ckb | 4 | 26 | 0.891 | 0.097 | 1.33E+00 | 4.88E−179 | 8.64E−172 |
| Tmem176a | 4 | 29 | 0.979 | 0.034 | 1.96E+00 | 8.35E−204 | 1.48E−196 |
| Tmem176b | 4 | 29 | 0.976 | 0.047 | 1.90E+00 | 1.75E−193 | 3.10E−186 |
| Fcer1g | 4 | 29 | 0.98 | 0.027 | 1.78E+00 | 2.57E−174 | 4.54E−167 |
| Fcer1g | 4 | 30 | 0.98 | 0.074 | 1.73E+00 | 1.68E−162 | 2.97E−155 |
| Tmem176a | 4 | 30 | 0.979 | 0.081 | 1.91E+00 | 1.28E−161 | 2.27E−154 |
| Trbc2 | 5 | 6 | 0.958 | 0.084 | 1.80E+00 | 0.00E+00 | 0.00E+00 |
| Ms4a4b | 5 | 6 | 0.935 | 0.057 | 1.49E+00 | 0.00E+00 | 0.00E+00 |
| Cd3d | 5 | 6 | 0.934 | 0.026 | 1.40E+00 | 0.00E+00 | 0.00E+00 |
| Cd3g | 5 | 6 | 0.942 | 0.024 | 1.39E+00 | 0.00E+00 | 0.00E+00 |
| Cd3e | 5 | 6 | 0.912 | 0.02 | 1.29E+00 | 0.00E+00 | 0.00E+00 |
| Lef1 | 5 | 6 | 0.818 | 0.011 | 1.10E+00 | 0.00E+00 | 0.00E+00 |
| Trac | 5 | 6 | 0.796 | 0.013 | 1.10E+00 | 0.00E+00 | 0.00E+00 |
| Lck | 5 | 6 | 0.802 | 0.098 | 9.17E−01 | 0.00E+00 | 0.00E+00 |
| Lat | 5 | 6 | 0.797 | 0.098 | 9.15E−01 | 0.00E+00 | 0.00E+00 |
| Thy1 | 5 | 6 | 0.793 | 0.092 | 9.78E−01 | 6.4E−316 | 0.00E+00 |
| Skap1 | 5 | 6 | 0.706 | 0.033 | 7.74E−01 | 1.37E−278 | 2.43E−271 |
| Igfbp4 | 5 | 6 | 0.555 | 0.014 | 8.95E−01 | 5.40E−273 | 9.57E−266 |
| Dapl1 | 5 | 6 | 0.55 | 0.006 | 1.04E+00 | 1.26E−256 | 2.23E−249 |
| Cd2 | 5 | 6 | 0.613 | 0.031 | 6.73E−01 | 1.79E−253 | 3.16E−246 |
| Klk8 | 5 | 6 | 0.572 | 0.03 | 6.42E−01 | 2.89E−242 | 5.11E−235 |
| Arl4c | 5 | 6 | 0.626 | 0.062 | 6.86E−01 | 2.80E−232 | 4.95E−225 |
| Trbc1 | 5 | 6 | 0.631 | 0.07 | 1.29E+00 | 1.70E−208 | 3.00E−201 |
| Cd247 | 5 | 6 | 0.514 | 0.011 | 5.38E−01 | 2.34E−203 | 4.14E−196 |
| Tcf7 | 5 | 6 | 0.555 | 0.058 | 5.79E−01 | 3.71E−177 | 6.57E−170 |
| Dusp2 | 5 | 6 | 0.5 | 0.098 | 6.04E−01 | 3.25E−168 | 5.76E−161 |
| Txk | 5 | 6 | 0.547 | 0.076 | 5.71E−01 | 3.11E−162 | 5.50E−155 |
| Cd8b1 | 5 | 7 | 0.947 | 0.082 | 1.49E+00 | 0.00E+00 | 0.00E+00 |
| Cd8a | 5 | 7 | 0.799 | 0.035 | 1.00E+00 | 0.00E+00 | 0.00E+00 |
| Nkg7 | 5 | 7 | 0.818 | 0.095 | 9.24E−01 | 0.00E+00 | 0.00E+00 |
| Dapl1 | 5 | 7 | 0.55 | 0.047 | 8.85E−01 | 0.00E+00 | 0.00E+00 |
| Igfbp4 | 5 | 7 | 0.555 | 0.058 | 7.98E−01 | 0.00E+00 | 0.00E+00 |
| Cd8b1 | 5 | 8 | 0.947 | 0.033 | 1.63E+00 | 0.00E+00 | 0.00E+00 |
| Cd3e | 5 | 8 | 0.912 | 0.016 | 1.30E+00 | 0.00E+00 | 0.00E+00 |
| Cd8a | 5 | 8 | 0.799 | 0.014 | 1.07E+00 | 0.00E+00 | 0.00E+00 |
| Trac | 5 | 8 | 0.796 | 0.056 | 1.05E+00 | 0.00E+00 | 0.00E+00 |
| Dapl1 | 5 | 8 | 0.55 | 0.001 | 1.04E+00 | 0.00E+00 | 0.00E+00 |
| Igfbp4 | 5 | 8 | 0.555 | 0.013 | 8.79E−01 | 0.00E+00 | 0.00E+00 |
| Rgs10 | 5 | 8 | 0.626 | 0.099 | 6.67E−01 | 0.00E+00 | 0.00E+00 |
| Klk8 | 5 | 8 | 0.572 | 0.076 | 5.86E−01 | 0.00E+00 | 0.00E+00 |
| Bcl2 | 5 | 9 | 0.762 | 0.095 | 1.27E+00 | 7.72E−296 | 1.37E−288 |
| Nkg7 | 5 | 9 | 0.818 | 0.068 | 1.15E+00 | 3.37E−277 | 5.96E−270 |
| Trac | 5 | 9 | 0.796 | 0.083 | 1.02E+00 | 7.53E−267 | 1.33E−259 |
| Lef1 | 5 | 9 | 0.818 | 0.057 | 1.04E+00 | 8.04E−257 | 1.42E−249 |
| Thy1 | 5 | 9 | 0.793 | 0.086 | 9.90E−01 | 1.90E−238 | 3.37E−231 |
| Cd8a | 5 | 9 | 0.799 | 0.031 | 1.05E+00 | 4.61E−213 | 8.17E−206 |
| Skap1 | 5 | 9 | 0.706 | 0.088 | 7.18E−01 | 2.66E−183 | 4.71E−176 |
| Igfbp4 | 5 | 9 | 0.555 | 0.066 | 8.38E−01 | 1.30E−179 | 2.29E−172 |
| Arl4c | 5 | 9 | 0.626 | 0.037 | 7.19E−01 | 4.23E−179 | 7.49E−172 |
| Dapl1 | 5 | 9 | 0.55 | 0.036 | 1.01E+00 | 4.26E−167 | 7.54E−160 |
| Satb1 | 5 | 9 | 0.618 | 0.069 | 6.56E−01 | 2.82E−164 | 4.98E−157 |
| Sell | 5 | 9 | 0.604 | 0.062 | 6.59E−01 | 8.62E−163 | 1.53E−155 |
| Klk8 | 5 | 9 | 0.572 | 0.034 | 6.35E−01 | 1.97E−160 | 3.48E−153 |
| S100a10 | 5 | 9 | 0.656 | 0.093 | 6.51E−01 | 3.53E−160 | 6.25E−153 |
| Cd8b1 | 5 | 10 | 0.947 | 0.027 | 1.63E+00 | 0.00E+00 | 0.00E+00 |
| Ms4a4b | 5 | 10 | 0.935 | 0.07 | 1.48E+00 | 0.00E+00 | 0.00E+00 |
| Cd3g | 5 | 10 | 0.942 | 0.055 | 1.36E+00 | 0.00E+00 | 0.00E+00 |
| Cd3d | 5 | 10 | 0.934 | 0.083 | 1.35E+00 | 0.00E+00 | 0.00E+00 |
| Bcl2 | 5 | 10 | 0.762 | 0.05 | 1.33E+00 | 0.00E+00 | 0.00E+00 |
| Cd3e | 5 | 10 | 0.912 | 0.07 | 1.25E+00 | 0.00E+00 | 0.00E+00 |
| Nkg7 | 5 | 10 | 0.818 | 0.045 | 1.18E+00 | 0.00E+00 | 0.00E+00 |
| Lef1 | 5 | 10 | 0.818 | 0.031 | 1.08E+00 | 0.00E+00 | 0.00E+00 |
| Trac | 5 | 10 | 0.796 | 0.05 | 1.07E+00 | 0.00E+00 | 0.00E+00 |
| Cd8a | 5 | 10 | 0.799 | 0.023 | 1.06E+00 | 0.00E+00 | 0.00E+00 |
| Thy1 | 5 | 10 | 0.793 | 0.049 | 1.04E+00 | 0.00E+00 | 0.00E+00 |
| Selplg | 5 | 10 | 0.825 | 0.09 | 1.01E+00 | 0.00E+00 | 0.00E+00 |
| Lat | 5 | 10 | 0.797 | 0.076 | 9.57E−01 | 0.00E+00 | 0.00E+00 |
| Ccnd2 | 5 | 10 | 0.75 | 0.044 | 9.54E−01 | 0.00E+00 | 0.00E+00 |
| Hcst | 5 | 10 | 0.782 | 0.089 | 9.37E−01 | 0.00E+00 | 0.00E+00 |
| Ccr7 | 5 | 10 | 0.678 | 0.069 | 9.00E−01 | 0.00E+00 | 0.00E+00 |
| Skap1 | 5 | 10 | 0.706 | 0.035 | 7.73E−01 | 0.00E+00 | 0.00E+00 |
| Arl4c | 5 | 10 | 0.626 | 0.017 | 7.42E−01 | 0.00E+00 | 0.00E+00 |
| Igfbp4 | 5 | 10 | 0.555 | 0.037 | 8.78E−01 | 7.8E−317 | 0.00E+00 |
| Sell | 5 | 10 | 0.604 | 0.042 | 6.80E−01 | 2.0E−315 | 3.62E−308 |
| Dapl1 | 5 | 10 | 0.55 | 0.013 | 1.03E+00 | 4.5E−315 | 8.06E−308 |
| Satb1 | 5 | 10 | 0.618 | 0.061 | 6.64E−01 | 3.05E−300 | 5.40E−293 |
| Fyb | 5 | 10 | 0.633 | 0.059 | 6.85E−01 | 3.34E−299 | 5.92E−292 |
| Trbc1 | 5 | 10 | 0.631 | 0.064 | 1.32E+00 | 2.06E−298 | 3.65E−291 |
| Itgb7 | 5 | 10 | 0.608 | 0.058 | 6.22E−01 | 1.31E−284 | 2.31E−277 |
| Cd2 | 5 | 10 | 0.613 | 0.074 | 6.13E−01 | 9.74E−273 | 1.72E−265 |
| Ramp1 | 5 | 10 | 0.528 | 0.019 | 5.78E−01 | 7.85E−268 | 1.39E−260 |
| Tcf7 | 5 | 10 | 0.555 | 0.029 | 6.15E−01 | 3.02E−264 | 5.34E−257 |
| Txk | 5 | 10 | 0.547 | 0.03 | 6.25E−01 | 1.63E−243 | 2.88E−236 |
| Cd247 | 5 | 10 | 0.514 | 0.025 | 5.25E−01 | 6.23E−243 | 1.10E−235 |
| Ifi203 | 5 | 10 | 0.515 | 0.023 | 5.53E−01 | 1.57E−230 | 2.77E−223 |
| Fam189b | 5 | 10 | 0.506 | 0.025 | 5.28E−01 | 1.71E−229 | 3.03E−222 |
| Mndal | 5 | 10 | 0.506 | 0.024 | 5.35E−01 | 1.07E−223 | 1.90E−216 |
| Emp3 | 5 | 10 | 0.574 | 0.071 | 5.97E−01 | 2.13E−222 | 3.77E−215 |
| H2-Q7 | 5 | 10 | 0.549 | 0.101 | 5.17E−01 | 1.25E−210 | 2.21E−203 |
| Cd8b1 | 5 | 11 | 0.947 | 0.018 | 1.64E+00 | 0.00E+00 | 0.00E+00 |
| Cd3d | 5 | 11 | 0.934 | 0.047 | 1.37E+00 | 0.00E+00 | 0.00E+00 |
| Cd3e | 5 | 11 | 0.912 | 0.021 | 1.28E+00 | 0.00E+00 | 0.00E+00 |
| Cd3g | 5 | 11 | 0.942 | 0.069 | 1.27E+00 | 0.00E+00 | 0.00E+00 |
| Lef1 | 5 | 11 | 0.818 | 0.058 | 1.05E+00 | 0.00E+00 | 0.00E+00 |
| Cd8a | 5 | 11 | 0.799 | 0.014 | 1.05E+00 | 0.00E+00 | 0.00E+00 |
| Ccr7 | 5 | 11 | 0.678 | 0.035 | 9.49E−01 | 0.00E+00 | 0.00E+00 |
| Dapl1 | 5 | 11 | 0.55 | 0 | 1.05E+00 | 9.92E−289 | 1.76E−281 |
| Igfbp4 | 5 | 11 | 0.555 | 0.009 | 9.05E−01 | 7.67E−283 | 1.36E−275 |
| Sell | 5 | 11 | 0.604 | 0.094 | 6.14E−01 | 8.04E−216 | 1.42E−208 |
| Cd8b1 | 5 | 12 | 0.947 | 0.03 | 1.62E+00 | 0.00E+00 | 0.00E+00 |
| Ms4a4b | 5 | 12 | 0.935 | 0.051 | 1.49E+00 | 0.00E+00 | 0.00E+00 |
| Cd3d | 5 | 12 | 0.934 | 0.028 | 1.40E+00 | 0.00E+00 | 0.00E+00 |
| Cd3e | 5 | 12 | 0.912 | 0.057 | 1.25E+00 | 0.00E+00 | 0.00E+00 |
| Trac | 5 | 12 | 0.796 | 0.014 | 1.10E+00 | 0.00E+00 | 0.00E+00 |
| Cd8a | 5 | 12 | 0.799 | 0.02 | 1.06E+00 | 0.00E+00 | 0.00E+00 |
| Lef1 | 5 | 12 | 0.818 | 0.073 | 1.03E+00 | 0.00E+00 | 0.00E+00 |
| Ms4a6b | 5 | 12 | 0.799 | 0.08 | 9.20E−01 | 0.00E+00 | 0.00E+00 |
| Ccr7 | 5 | 12 | 0.678 | 0.047 | 9.18E−01 | 5.14E−295 | 9.10E−288 |
| Cd2 | 5 | 12 | 0.613 | 0.026 | 6.79E−01 | 1.75E−271 | 3.09E−264 |
| Dapl1 | 5 | 12 | 0.55 | 0.003 | 1.04E+00 | 9.79E−262 | 1.73E−254 |
| Arl4c | 5 | 12 | 0.626 | 0.048 | 7.07E−01 | 1.32E−252 | 2.34E−245 |
| Igfbp4 | 5 | 12 | 0.555 | 0.03 | 8.47E−01 | 2.74E−243 | 4.85E−236 |
| Sell | 5 | 12 | 0.604 | 0.081 | 6.35E−01 | 9.27E−220 | 1.64E−212 |
| Cd247 | 5 | 12 | 0.514 | 0.027 | 5.20E−01 | 9.38E−193 | 1.66E−185 |
| Cd3d | 5 | 14 | 0.934 | 0.061 | 1.33E+00 | 1.69E−223 | 2.99E−216 |
| Cd8b1 | 5 | 14 | 0.947 | 0.061 | 1.58E+00 | 1.25E−210 | 2.22E−203 |
| Cd3e | 5 | 14 | 0.912 | 0.038 | 1.24E+00 | 1.22E−208 | 2.15E−201 |
| Cd3g | 5 | 14 | 0.942 | 0.058 | 1.30E+00 | 2.71E−192 | 4.79E−185 |
| Lat | 5 | 14 | 0.797 | 0.053 | 9.52E−01 | 1.02E−187 | 1.80E−180 |
| Trac | 5 | 14 | 0.796 | 0.033 | 1.06E+00 | 8.61E−187 | 1.52E−179 |
| Lef1 | 5 | 14 | 0.818 | 0.033 | 1.06E+00 | 2.95E−168 | 5.22E−161 |
| Cd8a | 5 | 14 | 0.799 | 0.019 | 1.06E+00 | 2.46E−164 | 4.35E−157 |
| Thy1 | 5 | 14 | 0.793 | 0.085 | 9.85E−01 | 2.27E−162 | 4.01E−155 |
| Cd8b1 | 5 | 15 | 0.947 | 0.008 | 1.65E+00 | 1.82E−197 | 3.22E−190 |
| Cd3d | 5 | 15 | 0.934 | 0.013 | 1.42E+00 | 2.40E−165 | 4.25E−158 |
| Cd3g | 5 | 15 | 0.942 | 0.04 | 1.36E+00 | 9.49E−165 | 1.68E−157 |
| Cd8b1 | 5 | 17 | 0.947 | 0.062 | 1.52E+00 | 2.23E−258 | 3.95E−251 |
| Lef1 | 5 | 17 | 0.818 | 0.062 | 1.03E+00 | 8.96E−196 | 1.59E−188 |
| Cd8b1 | 5 | 20 | 0.947 | 0.064 | 1.59E+00 | 1.42E−204 | 2.52E−197 |
| Cd3d | 5 | 20 | 0.934 | 0.093 | 1.25E+00 | 2.63E−193 | 4.66E−186 |
| Cd3e | 5 | 20 | 0.912 | 0.064 | 1.18E+00 | 2.15E−191 | 3.80E−184 |
| Cd3g | 5 | 20 | 0.942 | 0.081 | 1.28E+00 | 1.05E−188 | 1.86E−181 |
| Cd3d | 5 | 22 | 0.934 | 0.022 | 1.41E+00 | 1.13E−183 | 2.00E−176 |
| Cd8b1 | 5 | 22 | 0.947 | 0.043 | 1.62E+00 | 5.11E−174 | 9.05E−167 |
| Cd3e | 5 | 22 | 0.912 | 0.039 | 1.28E+00 | 1.04E−169 | 1.84E−162 |
| Cd3g | 5 | 22 | 0.942 | 0.039 | 1.37E+00 | 2.02E−166 | 3.58E−159 |
| Cd8b1 | 5 | 24 | 0.947 | 0.031 | 1.62E+00 | 6.07E−184 | 1.07E−176 |
| Ms4a4b | 5 | 24 | 0.935 | 0.031 | 1.52E+00 | 1.46E−180 | 2.58E−173 |
| Cd3e | 5 | 24 | 0.912 | 0.004 | 1.32E+00 | 5.08E−180 | 8.99E−173 |
| Cd3d | 5 | 24 | 0.934 | 0.018 | 1.42E+00 | 4.87E−179 | 8.62E−172 |
| Trbc2 | 5 | 24 | 0.958 | 0.081 | 1.76E+00 | 5.81E−171 | 1.03E−163 |
| Cd3g | 5 | 24 | 0.942 | 0.027 | 1.33E+00 | 3.96E−170 | 7.02E−163 |
| Nkg7 | 5 | 24 | 0.818 | 0.076 | 1.10E+00 | 1.16E−163 | 2.05E−156 |
| Coro1a | 5 | 25 | 0.982 | 0.1 | 1.63E+00 | 5.40E−263 | 9.56E−256 |
| Trbc2 | 5 | 25 | 0.958 | 0.063 | 1.81E+00 | 1.45E−233 | 2.56E−226 |
| Limd2 | 5 | 25 | 0.951 | 0.1 | 1.46E+00 | 1.06E−232 | 1.88E−225 |
| Cd3d | 5 | 25 | 0.934 | 0.018 | 1.42E+00 | 1.37E−225 | 2.43E−218 |
| Cd8b1 | 5 | 25 | 0.947 | 0.023 | 1.61E+00 | 2.35E−225 | 4.15E−218 |
| Ms4a4b | 5 | 25 | 0.935 | 0.018 | 1.54E+00 | 6.53E−221 | 1.16E−213 |
| Cd3g | 5 | 25 | 0.942 | 0.027 | 1.39E+00 | 1.93E−218 | 3.42E−211 |
| Cd3e | 5 | 25 | 0.912 | 0.009 | 1.31E+00 | 1.51E−212 | 2.67E−205 |
| Ptprcap | 5 | 25 | 0.844 | 0.063 | 1.11E+00 | 2.09E−160 | 3.69E−153 |
| Ms4a4b | 5 | 26 | 0.935 | 0.06 | 1.48E+00 | 2.56E−197 | 4.52E−190 |
| Cd3d | 5 | 26 | 0.934 | 0.032 | 1.39E+00 | 5.62E−191 | 9.95E−184 |
| Cd3g | 5 | 26 | 0.942 | 0.014 | 1.39E+00 | 1.36E−180 | 2.40E−173 |
| Cd3e | 5 | 26 | 0.912 | 0.078 | 1.22E+00 | 2.76E−180 | 4.89E−173 |
| Trbc2 | 5 | 26 | 0.958 | 0.083 | 1.77E+00 | 8.90E−180 | 1.58E−172 |
| Cd8b1 | 5 | 26 | 0.947 | 0.046 | 1.55E+00 | 9.54E−180 | 1.69E−172 |
| Trbc2 | 5 | 30 | 0.958 | 0.059 | 1.82E+00 | 2.56E−158 | 4.53E−151 |
| Cd52 | 5 | 32 | 0.992 | 0.094 | 2.05E+00 | 8.42E−188 | 1.49E−180 |
| Coro1a | 5 | 32 | 0.982 | 0.085 | 1.66E+00 | 4.24E−162 | 7.51E−155 |
| Tyrobp | 6 | 7 | 0.999 | 0.04 | 3.69E+00 | 0.00E+00 | 0.00E+00 |
| Plac8 | 6 | 7 | 0.999 | 0.054 | 3.41E+00 | 0.00E+00 | 0.00E+00 |
| Ccl4 | 6 | 7 | 0.868 | 0.016 | 3.40E+00 | 0.00E+00 | 0.00E+00 |
| Ly6c2 | 6 | 7 | 0.997 | 0.015 | 3.18E+00 | 0.00E+00 | 0.00E+00 |
| Irf8 | 6 | 7 | 0.997 | 0.07 | 3.02E+00 | 0.00E+00 | 0.00E+00 |
| Siglech | 6 | 7 | 0.998 | 0.008 | 2.78E+00 | 0.00E+00 | 0.00E+00 |
| Klk1 | 6 | 7 | 0.96 | 0.01 | 2.68E+00 | 0.00E+00 | 0.00E+00 |
| Ly6c1 | 6 | 7 | 0.991 | 0.007 | 2.62E+00 | 0.00E+00 | 0.00E+00 |
| Cox6a2 | 6 | 7 | 0.969 | 0.005 | 2.48E+00 | 0.00E+00 | 0.00E+00 |
| Rnase6 | 6 | 7 | 0.992 | 0.009 | 2.35E+00 | 0.00E+00 | 0.00E+00 |
| Iglc3 | 6 | 7 | 0.968 | 0.055 | 2.34E+00 | 0.00E+00 | 0.00E+00 |
| Ctsh | 6 | 7 | 0.988 | 0.03 | 2.27E+00 | 0.00E+00 | 0.00E+00 |
| Mpeg1 | 6 | 7 | 0.983 | 0.004 | 2.09E+00 | 0.00E+00 | 0.00E+00 |
| Ctsl | 6 | 7 | 0.96 | 0.098 | 2.01E+00 | 0.00E+00 | 0.00E+00 |
| D13Ertd608e | 6 | 7 | 0.924 | 0.001 | 1.94E+00 | 0.00E+00 | 0.00E+00 |
| Pld4 | 6 | 7 | 0.975 | 0.013 | 1.94E+00 | 0.00E+00 | 0.00E+00 |
| Tcf4 | 6 | 7 | 0.977 | 0.018 | 1.92E+00 | 0.00E+00 | 0.00E+00 |
| Klk1b27 | 6 | 7 | 0.965 | 0.012 | 1.91E+00 | 0.00E+00 | 0.00E+00 |
| Cybb | 6 | 7 | 0.978 | 0.09 | 1.87E+00 | 0.00E+00 | 0.00E+00 |
| Ccr9 | 6 | 7 | 0.956 | 0.09 | 1.76E+00 | 0.00E+00 | 0.00E+00 |
| Slpi | 6 | 7 | 0.789 | 0.013 | 1.68E+00 | 0.00E+00 | 0.00E+00 |
| Clec12a | 6 | 7 | 0.93 | 0.002 | 1.66E+00 | 0.00E+00 | 0.00E+00 |
| Tsc22d1 | 6 | 7 | 0.908 | 0.032 | 1.64E+00 | 0.00E+00 | 0.00E+00 |
| Mef2c | 6 | 7 | 0.947 | 0.019 | 1.63E+00 | 0.00E+00 | 0.00E+00 |
| Lair1 | 6 | 7 | 0.937 | 0.008 | 1.59E+00 | 0.00E+00 | 0.00E+00 |
| Alox5ap | 6 | 7 | 0.937 | 0.004 | 1.58E+00 | 0.00E+00 | 0.00E+00 |
| Fcer1g | 6 | 7 | 0.917 | 0.019 | 1.55E+00 | 0.00E+00 | 0.00E+00 |
| Unc93b1 | 6 | 7 | 0.934 | 0.065 | 1.50E+00 | 0.00E+00 | 0.00E+00 |
| Bcl11a | 6 | 7 | 0.889 | 0.007 | 1.44E+00 | 0.00E+00 | 0.00E+00 |
| Ncf1 | 6 | 7 | 0.861 | 0.019 | 1.43E+00 | 0.00E+00 | 0.00E+00 |
| P2ry14 | 6 | 7 | 0.874 | 0.006 | 1.40E+00 | 0.00E+00 | 0.00E+00 |
| Pltp | 6 | 7 | 0.842 | 0.004 | 1.32E+00 | 0.00E+00 | 0.00E+00 |
| Smim5 | 6 | 7 | 0.781 | 0.001 | 1.32E+00 | 0.00E+00 | 0.00E+00 |
| Upb1 | 6 | 7 | 0.86 | 0.008 | 1.29E+00 | 0.00E+00 | 0.00E+00 |
| Napsa | 6 | 7 | 0.849 | 0.019 | 1.28E+00 | 0.00E+00 | 0.00E+00 |
| Ppfia4 | 6 | 7 | 0.864 | 0.002 | 1.24E+00 | 0.00E+00 | 0.00E+00 |
| Nucb2 | 6 | 7 | 0.868 | 0.043 | 1.22E+00 | 0.00E+00 | 0.00E+00 |
| Spib | 6 | 7 | 0.823 | 0.021 | 1.21E+00 | 0.00E+00 | 0.00E+00 |
| Cadm1 | 6 | 7 | 0.848 | 0.001 | 1.18E+00 | 0.00E+00 | 0.00E+00 |
| Cd68 | 6 | 7 | 0.83 | 0.01 | 1.17E+00 | 0.00E+00 | 0.00E+00 |
| Runx2 | 6 | 7 | 0.811 | 0.023 | 1.17E+00 | 0.00E+00 | 0.00E+00 |
| Pkib | 6 | 7 | 0.782 | 0.004 | 1.15E+00 | 0.00E+00 | 0.00E+00 |
| Kctd12 | 6 | 7 | 0.81 | 0.02 | 1.15E+00 | 0.00E+00 | 0.00E+00 |
| Blnk | 6 | 7 | 0.812 | 0.022 | 1.12E+00 | 0.00E+00 | 0.00E+00 |
| Amica1 | 6 | 7 | 0.791 | 0.007 | 1.11E+00 | 0.00E+00 | 0.00E+00 |
| Kmo | 6 | 7 | 0.79 | 0.003 | 1.03E+00 | 0.00E+00 | 0.00E+00 |
| Gapt | 6 | 7 | 0.69 | 0.002 | 9.05E−01 | 0.00E+00 | 0.00E+00 |
| Sh3bgr | 6 | 7 | 0.7 | 0 | 9.59E−01 | 4.1E−312 | 7.41E−305 |
| Pkig | 6 | 7 | 0.839 | 0.041 | 1.13E+00 | 1.6E−311 | 2.85E−304 |
| Scimp | 6 | 7 | 0.785 | 0.019 | 1.06E+00 | 8.50E−308 | 1.51E−300 |
| Fcrla | 6 | 7 | 0.788 | 0.02 | 1.26E+00 | 8.04E−303 | 1.42E−295 |
| Lefty1 | 6 | 7 | 0.739 | 0.01 | 9.87E−01 | 5.71E−302 | 1.01E−294 |
| H2-DMb1 | 6 | 7 | 0.711 | 0.007 | 1.12E+00 | 1.26E−294 | 2.23E−287 |
| Hpse | 6 | 7 | 0.73 | 0.008 | 9.91E−01 | 1.52E−294 | 2.68E−287 |
| Ccr2 | 6 | 7 | 0.777 | 0.046 | 1.17E+00 | 4.98E−290 | 8.82E−283 |
| Zeb2 | 6 | 7 | 0.725 | 0.022 | 9.78E−01 | 2.15E−287 | 3.81E−280 |
| Pir | 6 | 7 | 0.67 | 0.001 | 1.01E+00 | 4.22E−276 | 7.48E−269 |
| Atp2a1 | 6 | 7 | 0.654 | 0.002 | 8.16E−01 | 5.80E−275 | 1.03E−267 |
| Klrd1 | 6 | 7 | 0.685 | 0.007 | 8.81E−01 | 8.69E−275 | 1.54E−267 |
| Mzb1 | 6 | 7 | 0.81 | 0.056 | 1.35E+00 | 5.34E−273 | 9.45E−266 |
| Ifi30 | 6 | 7 | 0.846 | 0.072 | 1.40E+00 | 3.49E−267 | 6.18E−260 |
| Lgals3 | 6 | 7 | 0.756 | 0.03 | 1.16E+00 | 7.20E−261 | 1.27E−253 |
| I830077J02Rik | 6 | 7 | 0.63 | 0 | 8.08E−01 | 3.03E−260 | 5.36E−253 |
| Cd209a | 6 | 7 | 0.578 | 0 | 1.25E+00 | 4.68E−251 | 8.29E−244 |
| Tubgcp5 | 6 | 7 | 0.744 | 0.04 | 9.82E−01 | 1.35E−243 | 2.39E−236 |
| Cd209d | 6 | 7 | 0.564 | 0.001 | 1.30E+00 | 1.68E−242 | 2.98E−235 |
| Tbc1d8 | 6 | 7 | 0.593 | 0.002 | 7.06E−01 | 7.43E−239 | 1.32E−231 |
| Plekhm3 | 6 | 7 | 0.635 | 0.013 | 7.47E−01 | 1.32E−238 | 2.33E−231 |
| Cd180 | 6 | 7 | 0.627 | 0.012 | 7.71E−01 | 4.96E−238 | 8.78E−231 |
| Lyn | 6 | 7 | 0.681 | 0.017 | 8.20E−01 | 7.23E−237 | 1.28E−229 |
| Abhd17b | 6 | 7 | 0.78 | 0.076 | 1.07E+00 | 8.27E−237 | 1.46E−229 |
| Slamf9 | 6 | 7 | 0.592 | 0.002 | 8.00E−01 | 9.18E−236 | 1.62E−228 |
| Sdc4 | 6 | 7 | 0.676 | 0.041 | 8.52E−01 | 4.11E−234 | 7.28E−227 |
| Klra17 | 6 | 7 | 0.57 | 0.001 | 7.70E−01 | 2.06E−231 | 3.65E−224 |
| Csf2rb | 6 | 7 | 0.577 | 0.001 | 6.97E−01 | 6.95E−228 | 1.23E−220 |
| Plek | 6 | 7 | 0.634 | 0.017 | 8.31E−01 | 7.06E−225 | 1.25E−217 |
| Lifr | 6 | 7 | 0.543 | 0 | 6.51E−01 | 1.41E−224 | 2.50E−217 |
| Rell1 | 6 | 7 | 0.586 | 0.005 | 7.41E−01 | 3.01E−223 | 5.32E−216 |
| Pgr | 6 | 7 | 0.623 | 0.01 | 7.38E−01 | 1.35E−222 | 2.38E−215 |
| Lat2 | 6 | 7 | 0.644 | 0.019 | 7.70E−01 | 3.62E−215 | 6.41E−208 |
| Gsn | 6 | 7 | 0.819 | 0.08 | 1.09E+00 | 5.35E−215 | 9.47E−208 |
| Havcr1 | 6 | 7 | 0.568 | 0.005 | 6.33E−01 | 1.48E−214 | 2.62E−207 |
| Spns3 | 6 | 7 | 0.564 | 0.002 | 6.76E−01 | 2.36E−213 | 4.17E−206 |
| Hs3st1 | 6 | 7 | 0.551 | 0.006 | 7.40E−01 | 4.11E−201 | 7.28E−194 |
| Lmo2 | 6 | 7 | 0.563 | 0.011 | 6.36E−01 | 6.60E−200 | 1.17E−192 |
| Pafah1b3 | 6 | 7 | 0.776 | 0.086 | 9.60E−01 | 2.03E−195 | 3.60E−188 |
| Rpgrip1 | 6 | 7 | 0.5 | 0.005 | 6.57E−01 | 2.36E−194 | 4.17E−187 |
| Plaur | 6 | 7 | 0.561 | 0.019 | 9.97E−01 | 1.31E−193 | 2.32E−186 |
| Flt3 | 6 | 7 | 0.51 | 0.002 | 5.96E−01 | 3.20E−190 | 5.67E−183 |
| Hhex | 6 | 7 | 0.553 | 0.011 | 6.88E−01 | 2.52E−187 | 4.46E−180 |
| Klrk1 | 6 | 7 | 0.522 | 0.009 | 6.98E−01 | 8.33E−183 | 1.47E−175 |
| Nek6 | 6 | 7 | 0.518 | 0.005 | 6.07E−01 | 3.08E−179 | 5.45E−172 |
| Mctp2 | 6 | 7 | 0.5 | 0.009 | 5.78E−01 | 1.98E−176 | 3.51E−169 |
| Hmgn3 | 6 | 7 | 0.6 | 0.033 | 6.78E−01 | 2.45E−171 | 4.33E−164 |
| Syk | 6 | 7 | 0.5 | 0.01 | 5.68E−01 | 5.95E−168 | 1.05E−160 |
| Tmem229b | 6 | 7 | 0.706 | 0.071 | 8.24E−01 | 3.39E−167 | 6.00E−160 |
| Gpr137b | 6 | 7 | 0.53 | 0.019 | 5.62E−01 | 3.22E−165 | 5.70E−158 |
| Irf5 | 6 | 7 | 0.61 | 0.038 | 6.73E−01 | 1.51E−164 | 2.68E−157 |
| Irf7 | 6 | 7 | 0.712 | 0.076 | 1.06E+00 | 2.37E−163 | 4.19E−156 |
| Trf | 6 | 7 | 0.513 | 0.017 | 6.03E−01 | 1.96E−158 | 3.47E−151 |
| Klk1 | 6 | 8 | 0.96 | 0.074 | 2.61E+00 | 0.00E+00 | 0.00E+00 |
| Cox6a2 | 6 | 8 | 0.969 | 0.044 | 2.41E+00 | 0.00E+00 | 0.00E+00 |
| Rnase6 | 6 | 8 | 0.992 | 0.035 | 2.33E+00 | 0.00E+00 | 0.00E+00 |
| Iglc3 | 6 | 8 | 0.968 | 0.04 | 2.28E+00 | 0.00E+00 | 0.00E+00 |
| Ctsh | 6 | 8 | 0.988 | 0.087 | 2.22E+00 | 0.00E+00 | 0.00E+00 |
| Ctsl | 6 | 8 | 0.96 | 0.053 | 2.11E+00 | 0.00E+00 | 0.00E+00 |
| Mpeg1 | 6 | 8 | 0.983 | 0.041 | 2.06E+00 | 0.00E+00 | 0.00E+00 |
| Cybb | 6 | 8 | 0.978 | 0.026 | 1.96E+00 | 0.00E+00 | 0.00E+00 |
| D13Ertd608e | 6 | 8 | 0.924 | 0.008 | 1.94E+00 | 0.00E+00 | 0.00E+00 |
| Klk1b27 | 6 | 8 | 0.965 | 0.016 | 1.90E+00 | 0.00E+00 | 0.00E+00 |
| Pld4 | 6 | 8 | 0.975 | 0.057 | 1.89E+00 | 0.00E+00 | 0.00E+00 |
| Ccr9 | 6 | 8 | 0.956 | 0.051 | 1.88E+00 | 0.00E+00 | 0.00E+00 |
| Clec12a | 6 | 8 | 0.93 | 0.02 | 1.64E+00 | 0.00E+00 | 0.00E+00 |
| Alox5ap | 6 | 8 | 0.937 | 0.029 | 1.55E+00 | 0.00E+00 | 0.00E+00 |
| Lair1 | 6 | 8 | 0.937 | 0.071 | 1.52E+00 | 0.00E+00 | 0.00E+00 |
| Bcl11a | 6 | 8 | 0.889 | 0.015 | 1.43E+00 | 0.00E+00 | 0.00E+00 |
| Nucb2 | 6 | 8 | 0.868 | 0.01 | 1.25E+00 | 0.00E+00 | 0.00E+00 |
| Ppfia4 | 6 | 8 | 0.864 | 0.014 | 1.23E+00 | 0.00E+00 | 0.00E+00 |
| Upb1 | 6 | 8 | 0.86 | 0.016 | 1.28E+00 | 1.67E−306 | 2.96E−299 |
| Cadm1 | 6 | 8 | 0.848 | 0.016 | 1.16E+00 | 2.18E−304 | 3.86E−297 |
| Spib | 6 | 8 | 0.823 | 0.017 | 1.21E+00 | 6.90E−290 | 1.22E−282 |
| Smim5 | 6 | 8 | 0.781 | 0.01 | 1.31E+00 | 2.86E−281 | 5.07E−274 |
| Gm2a | 6 | 8 | 0.91 | 0.092 | 1.37E+00 | 2.30E−274 | 4.08E−267 |
| Ifi27l2a | 6 | 8 | 0.844 | 0.054 | 2.11E+00 | 4.32E−272 | 7.66E−265 |
| P2ry14 | 6 | 8 | 0.874 | 0.058 | 1.34E+00 | 1.44E−261 | 2.55E−254 |
| Cd68 | 6 | 8 | 0.83 | 0.022 | 1.16E+00 | 1.42E−258 | 2.51E−251 |
| Blnk | 6 | 8 | 0.812 | 0.02 | 1.12E+00 | 1.20E−256 | 2.13E−249 |
| Mzb1 | 6 | 8 | 0.81 | 0.025 | 1.39E+00 | 2.40E−255 | 4.26E−248 |
| Pkib | 6 | 8 | 0.782 | 0.013 | 1.14E+00 | 1.14E−254 | 2.01E−247 |
| Slpi | 6 | 8 | 0.789 | 0.029 | 1.66E+00 | 1.77E−242 | 3.13E−235 |
| Fcrla | 6 | 8 | 0.788 | 0.015 | 1.27E+00 | 1.46E−237 | 2.58E−230 |
| Pltp | 6 | 8 | 0.842 | 0.043 | 1.28E+00 | 3.11E−233 | 5.52E−226 |
| Pacsin1 | 6 | 8 | 0.819 | 0.03 | 1.08E+00 | 2.15E−232 | 3.81E−225 |
| Lefty1 | 6 | 8 | 0.739 | 0.009 | 9.89E−01 | 5.65E−232 | 1.00E−224 |
| Kmo | 6 | 8 | 0.79 | 0.02 | 1.01E+00 | 5.62E−230 | 9.96E−223 |
| Sh3bgr | 6 | 8 | 0.7 | 0.005 | 9.54E−01 | 7.57E−224 | 1.34E−216 |
| Ly86 | 6 | 8 | 0.733 | 0.014 | 9.74E−01 | 1.93E−218 | 3.42E−211 |
| Gapt | 6 | 8 | 0.69 | 0.023 | 8.82E−01 | 1.65E−215 | 2.92E−208 |
| Ifi30 | 6 | 8 | 0.846 | 0.058 | 1.42E+00 | 1.18E−213 | 2.09E−206 |
| Amica1 | 6 | 8 | 0.791 | 0.04 | 1.07E+00 | 3.89E−205 | 6.90E−198 |
| Tnfrsf13b | 6 | 8 | 0.786 | 0.037 | 1.02E+00 | 5.71E−204 | 1.01E−196 |
| Bmyc | 6 | 8 | 0.807 | 0.041 | 1.07E+00 | 9.53E−204 | 1.69E−196 |
| H2-DMb1 | 6 | 8 | 0.711 | 0.018 | 1.11E+00 | 7.48E−201 | 1.32E−193 |
| Trib1 | 6 | 8 | 0.722 | 0.027 | 1.05E+00 | 1.24E−197 | 2.20E−190 |
| Gsn | 6 | 8 | 0.819 | 0.038 | 1.14E+00 | 8.13E−194 | 1.44E−186 |
| Atp2a1 | 6 | 8 | 0.654 | 0.01 | 8.08E−01 | 1.34E−189 | 2.38E−182 |
| Pir | 6 | 8 | 0.67 | 0.007 | 1.00E+00 | 5.75E−186 | 1.02E−178 |
| Cd180 | 6 | 8 | 0.627 | 0.011 | 7.71E−01 | 1.18E−182 | 2.09E−175 |
| Tubgcp5 | 6 | 8 | 0.744 | 0.046 | 9.75E−01 | 8.31E−181 | 1.47E−173 |
| Cd200 | 6 | 8 | 0.609 | 0.007 | 7.81E−01 | 1.45E−172 | 2.57E−165 |
| Slamf9 | 6 | 8 | 0.592 | 0.004 | 7.97E−01 | 3.45E−171 | 6.11E−164 |
| Cd209d | 6 | 8 | 0.564 | 0.011 | 1.29E+00 | 3.78E−166 | 6.69E−159 |
| Hpse | 6 | 8 | 0.73 | 0.041 | 9.58E−01 | 1.70E−165 | 3.00E−158 |
| I830077J02Rik | 6 | 8 | 0.63 | 0.012 | 7.96E−01 | 3.02E−162 | 5.34E−155 |
| Havcr1 | 6 | 8 | 0.568 | 0.007 | 6.31E−01 | 2.53E−159 | 4.47E−152 |
| Tbc1d8 | 6 | 8 | 0.593 | 0.011 | 6.96E−01 | 5.35E−159 | 9.47E−152 |
| Tyrobp | 6 | 9 | 0.999 | 0.093 | 3.64E+00 | 0.00E+00 | 0.00E+00 |
| Ccl4 | 6 | 9 | 0.868 | 0.033 | 3.40E+00 | 0.00E+00 | 0.00E+00 |
| Bst2 | 6 | 9 | 0.998 | 0.099 | 3.31E+00 | 0.00E+00 | 0.00E+00 |
| Ly6c2 | 6 | 9 | 0.997 | 0.036 | 3.16E+00 | 0.00E+00 | 0.00E+00 |
| Siglech | 6 | 9 | 0.998 | 0.022 | 2.77E+00 | 0.00E+00 | 0.00E+00 |
| Klk1 | 6 | 9 | 0.96 | 0.021 | 2.67E+00 | 0.00E+00 | 0.00E+00 |
| Ly6c1 | 6 | 9 | 0.991 | 0.008 | 2.63E+00 | 0.00E+00 | 0.00E+00 |
| Cox6a2 | 6 | 9 | 0.969 | 0.013 | 2.48E+00 | 0.00E+00 | 0.00E+00 |
| Grn | 6 | 9 | 0.986 | 0.1 | 2.19E+00 | 0.00E+00 | 0.00E+00 |
| Ctsl | 6 | 9 | 0.96 | 0.021 | 2.14E+00 | 0.00E+00 | 0.00E+00 |
| Mpeg1 | 6 | 9 | 0.983 | 0.02 | 2.07E+00 | 0.00E+00 | 0.00E+00 |
| Cd7 | 6 | 9 | 0.954 | 0.077 | 1.94E+00 | 0.00E+00 | 0.00E+00 |
| D13Ertd608e | 6 | 9 | 0.924 | 0.002 | 1.94E+00 | 0.00E+00 | 0.00E+00 |
| Klk1b27 | 6 | 9 | 0.965 | 0.008 | 1.91E+00 | 0.00E+00 | 0.00E+00 |
| Ccr9 | 6 | 9 | 0.956 | 0.017 | 1.91E+00 | 0.00E+00 | 0.00E+00 |
| Sell | 6 | 9 | 0.973 | 0.062 | 1.82E+00 | 0.00E+00 | 0.00E+00 |
| Cd8b1 | 6 | 9 | 0.593 | 0.057 | 1.75E+00 | 0.00E+00 | 0.00E+00 |
| Tsc22d1 | 6 | 9 | 0.908 | 0.021 | 1.66E+00 | 0.00E+00 | 0.00E+00 |
| Lair1 | 6 | 9 | 0.937 | 0.008 | 1.59E+00 | 0.00E+00 | 0.00E+00 |
| S100a6 | 6 | 9 | 0.895 | 0.029 | 1.58E+00 | 0.00E+00 | 0.00E+00 |
| Alox5ap | 6 | 9 | 0.937 | 0.005 | 1.58E+00 | 0.00E+00 | 0.00E+00 |
| Fcer1g | 6 | 9 | 0.917 | 0.038 | 1.52E+00 | 0.00E+00 | 0.00E+00 |
| Atp1b1 | 6 | 9 | 0.897 | 0.016 | 1.50E+00 | 0.00E+00 | 0.00E+00 |
| Gpr171 | 6 | 9 | 0.847 | 0.081 | 1.47E+00 | 0.00E+00 | 0.00E+00 |
| Rilpl2 | 6 | 9 | 0.857 | 0.078 | 1.45E+00 | 0.00E+00 | 0.00E+00 |
| P2ry14 | 6 | 9 | 0.874 | 0.008 | 1.40E+00 | 0.00E+00 | 0.00E+00 |
| Smim5 | 6 | 9 | 0.781 | 0.003 | 1.32E+00 | 0.00E+00 | 0.00E+00 |
| Pltp | 6 | 9 | 0.842 | 0.015 | 1.31E+00 | 0.00E+00 | 0.00E+00 |
| Cd209d | 6 | 9 | 0.564 | 0.005 | 1.29E+00 | 0.00E+00 | 0.00E+00 |
| Ramp1 | 6 | 9 | 0.879 | 0.053 | 1.29E+00 | 0.00E+00 | 0.00E+00 |
| Upb1 | 6 | 9 | 0.86 | 0.005 | 1.29E+00 | 0.00E+00 | 0.00E+00 |
| Ccr2 | 6 | 9 | 0.777 | 0.004 | 1.26E+00 | 0.00E+00 | 0.00E+00 |
| Cd209a | 6 | 9 | 0.578 | 0.004 | 1.25E+00 | 0.00E+00 | 0.00E+00 |
| Ifnar2 | 6 | 9 | 0.868 | 0.023 | 1.24E+00 | 0.00E+00 | 0.00E+00 |
| Ppfia4 | 6 | 9 | 0.864 | 0.005 | 1.24E+00 | 0.00E+00 | 0.00E+00 |
| Nucb2 | 6 | 9 | 0.868 | 0.046 | 1.21E+00 | 0.00E+00 | 0.00E+00 |
| Runx2 | 6 | 9 | 0.811 | 0.009 | 1.19E+00 | 0.00E+00 | 0.00E+00 |
| Cadm1 | 6 | 9 | 0.848 | 0.003 | 1.18E+00 | 0.00E+00 | 0.00E+00 |
| Lgals3 | 6 | 9 | 0.756 | 0.035 | 1.15E+00 | 0.00E+00 | 0.00E+00 |
| Kctd12 | 6 | 9 | 0.81 | 0.017 | 1.15E+00 | 0.00E+00 | 0.00E+00 |
| Cd68 | 6 | 9 | 0.83 | 0.042 | 1.14E+00 | 0.00E+00 | 0.00E+00 |
| Amica1 | 6 | 9 | 0.791 | 0.009 | 1.11E+00 | 0.00E+00 | 0.00E+00 |
| Snx18 | 6 | 9 | 0.81 | 0.075 | 1.08E+00 | 0.00E+00 | 0.00E+00 |
| Plaur | 6 | 9 | 0.561 | 0.009 | 1.01E+00 | 0.00E+00 | 0.00E+00 |
| Pir | 6 | 9 | 0.67 | 0.003 | 1.00E+00 | 0.00E+00 | 0.00E+00 |
| Trib1 | 6 | 9 | 0.722 | 0.072 | 1.00E+00 | 0.00E+00 | 0.00E+00 |
| Il7r | 6 | 9 | 0.757 | 0.043 | 9.84E−01 | 0.00E+00 | 0.00E+00 |
| Zeb2 | 6 | 9 | 0.725 | 0.02 | 9.80E−01 | 0.00E+00 | 0.00E+00 |
| Sh3bgr | 6 | 9 | 0.7 | 0.005 | 9.53E−01 | 0.00E+00 | 0.00E+00 |
| Socs3 | 6 | 9 | 0.52 | 0.033 | 9.25E−01 | 0.00E+00 | 0.00E+00 |
| Mndal | 6 | 9 | 0.694 | 0.035 | 9.07E−01 | 0.00E+00 | 0.00E+00 |
| Ahnak | 6 | 9 | 0.688 | 0.022 | 8.99E−01 | 0.00E+00 | 0.00E+00 |
| Klrd1 | 6 | 9 | 0.685 | 0.015 | 8.78E−01 | 0.00E+00 | 0.00E+00 |
| Timp2 | 6 | 9 | 0.68 | 0.019 | 8.59E−01 | 0.00E+00 | 0.00E+00 |
| Gapt | 6 | 9 | 0.69 | 0.048 | 8.58E−01 | 0.00E+00 | 0.00E+00 |
| Sdc4 | 6 | 9 | 0.676 | 0.038 | 8.50E−01 | 0.00E+00 | 0.00E+00 |
| Gpr183 | 6 | 9 | 0.624 | 0.07 | 8.40E−01 | 0.00E+00 | 0.00E+00 |
| Sla2 | 6 | 9 | 0.67 | 0.026 | 8.33E−01 | 0.00E+00 | 0.00E+00 |
| Irf2bp2 | 6 | 9 | 0.712 | 0.101 | 8.18E−01 | 0.00E+00 | 0.00E+00 |
| Atp2a1 | 6 | 9 | 0.654 | 0.002 | 8.16E−01 | 0.00E+00 | 0.00E+00 |
| I830077J02Rik | 6 | 9 | 0.63 | 0.004 | 8.03E−01 | 0.00E+00 | 0.00E+00 |
| Slamf9 | 6 | 9 | 0.592 | 0.003 | 7.98E−01 | 0.00E+00 | 0.00E+00 |
| Irf1 | 6 | 9 | 0.65 | 0.092 | 7.96E−01 | 0.00E+00 | 0.00E+00 |
| Tmem229b | 6 | 9 | 0.706 | 0.1 | 7.91E−01 | 0.00E+00 | 0.00E+00 |
| Rnf149 | 6 | 9 | 0.601 | 0.075 | 7.79E−01 | 0.00E+00 | 0.00E+00 |
| Klra17 | 6 | 9 | 0.57 | 0.003 | 7.68E−01 | 0.00E+00 | 0.00E+00 |
| Zyx | 6 | 9 | 0.623 | 0.036 | 7.62E−01 | 0.00E+00 | 0.00E+00 |
| Adgre5 | 6 | 9 | 0.617 | 0.052 | 7.57E−01 | 0.00E+00 | 0.00E+00 |
| Ifi203 | 6 | 9 | 0.573 | 0.048 | 7.44E−01 | 0.00E+00 | 0.00E+00 |
| Rell1 | 6 | 9 | 0.586 | 0.021 | 7.24E−01 | 0.00E+00 | 0.00E+00 |
| Klrk1 | 6 | 9 | 0.522 | 0.005 | 7.05E−01 | 0.00E+00 | 0.00E+00 |
| Tbc1d8 | 6 | 9 | 0.593 | 0.003 | 7.04E−01 | 0.00E+00 | 0.00E+00 |
| Lag3 | 6 | 9 | 0.548 | 0.016 | 7.01E−01 | 0.00E+00 | 0.00E+00 |
| Itgb7 | 6 | 9 | 0.622 | 0.075 | 7.00E−01 | 0.00E+00 | 0.00E+00 |
| Csf2rb | 6 | 9 | 0.577 | 0.006 | 6.92E−01 | 0.00E+00 | 0.00E+00 |
| Trim30a | 6 | 9 | 0.597 | 0.067 | 6.84E−01 | 0.00E+00 | 0.00E+00 |
| Plekhm3 | 6 | 9 | 0.635 | 0.074 | 6.76E−01 | 0.00E+00 | 0.00E+00 |
| Spns3 | 6 | 9 | 0.564 | 0.009 | 6.69E−01 | 0.00E+00 | 0.00E+00 |
| Fgr | 6 | 9 | 0.623 | 0.081 | 6.65E−01 | 0.00E+00 | 0.00E+00 |
| Lifr | 6 | 9 | 0.543 | 0.006 | 6.45E−01 | 0.00E+00 | 0.00E+00 |
| Il10rb | 6 | 9 | 0.59 | 0.045 | 6.29E−01 | 0.00E+00 | 0.00E+00 |
| Trf | 6 | 9 | 0.513 | 0.005 | 6.19E−01 | 0.00E+00 | 0.00E+00 |
| Tex2 | 6 | 9 | 0.522 | 0.012 | 5.68E−01 | 0.00E+00 | 0.00E+00 |
| Runx3 | 6 | 9 | 0.567 | 0.097 | 6.09E−01 | 1.44E−305 | 2.54E−298 |
| Cmah | 6 | 9 | 0.546 | 0.077 | 5.36E−01 | 8.39E−296 | 1.48E−288 |
| Mctp2 | 6 | 9 | 0.5 | 0.038 | 5.49E−01 | 1.53E−293 | 2.71E−286 |
| Nek6 | 6 | 9 | 0.518 | 0.089 | 5.18E−01 | 3.80E−284 | 6.73E−277 |
| Rnf122 | 6 | 9 | 0.525 | 0.058 | 5.87E−01 | 1.18E−279 | 2.08E−272 |
| Rpgrip1 | 6 | 9 | 0.5 | 0.039 | 6.19E−01 | 8.44E−279 | 1.50E−271 |
| Bhlhe40 | 6 | 9 | 0.504 | 0.05 | 6.30E−01 | 2.55E−276 | 4.51E−269 |
| Tyrobp | 6 | 10 | 0.999 | 0.064 | 3.65E+00 | 0.00E+00 | 0.00E+00 |
| Ccl4 | 6 | 10 | 0.868 | 0.025 | 3.41E+00 | 0.00E+00 | 0.00E+00 |
| Plac8 | 6 | 10 | 0.999 | 0.063 | 3.39E+00 | 0.00E+00 | 0.00E+00 |
| Bst2 | 6 | 10 | 0.998 | 0.051 | 3.36E+00 | 0.00E+00 | 0.00E+00 |
| Ly6c2 | 6 | 10 | 0.997 | 0.019 | 3.17E+00 | 0.00E+00 | 0.00E+00 |
| Siglech | 6 | 10 | 0.998 | 0.011 | 2.78E+00 | 0.00E+00 | 0.00E+00 |
| Klk1 | 6 | 10 | 0.96 | 0.014 | 2.67E+00 | 0.00E+00 | 0.00E+00 |
| Ly6c1 | 6 | 10 | 0.991 | 0.007 | 2.62E+00 | 0.00E+00 | 0.00E+00 |
| Cox6a2 | 6 | 10 | 0.969 | 0.008 | 2.48E+00 | 0.00E+00 | 0.00E+00 |
| Grn | 6 | 10 | 0.986 | 0.044 | 2.26E+00 | 0.00E+00 | 0.00E+00 |
| Ctsl | 6 | 10 | 0.96 | 0.016 | 2.14E+00 | 0.00E+00 | 0.00E+00 |
| Mpeg1 | 6 | 10 | 0.983 | 0.015 | 2.08E+00 | 0.00E+00 | 0.00E+00 |
| Cd7 | 6 | 10 | 0.954 | 0.043 | 1.97E+00 | 0.00E+00 | 0.00E+00 |
| D13Ertd608e | 6 | 10 | 0.924 | 0.001 | 1.94E+00 | 0.00E+00 | 0.00E+00 |
| Ccr9 | 6 | 10 | 0.956 | 0.008 | 1.93E+00 | 0.00E+00 | 0.00E+00 |
| Selplg | 6 | 10 | 0.983 | 0.09 | 1.92E+00 | 0.00E+00 | 0.00E+00 |
| Klk1b27 | 6 | 10 | 0.965 | 0.005 | 1.92E+00 | 0.00E+00 | 0.00E+00 |
| Sell | 6 | 10 | 0.973 | 0.042 | 1.84E+00 | 0.00E+00 | 0.00E+00 |
| Tsc22d1 | 6 | 10 | 0.908 | 0.015 | 1.67E+00 | 0.00E+00 | 0.00E+00 |
| S100a6 | 6 | 10 | 0.895 | 0.01 | 1.61E+00 | 0.00E+00 | 0.00E+00 |
| Lair1 | 6 | 10 | 0.937 | 0.004 | 1.59E+00 | 0.00E+00 | 0.00E+00 |
| Alox5ap | 6 | 10 | 0.937 | 0.005 | 1.58E+00 | 0.00E+00 | 0.00E+00 |
| Fyb | 6 | 10 | 0.944 | 0.059 | 1.52E+00 | 0.00E+00 | 0.00E+00 |
| Fcer1g | 6 | 10 | 0.917 | 0.032 | 1.52E+00 | 0.00E+00 | 0.00E+00 |
| Atp1b1 | 6 | 10 | 0.897 | 0.009 | 1.51E+00 | 0.00E+00 | 0.00E+00 |
| Rilpl2 | 6 | 10 | 0.857 | 0.049 | 1.49E+00 | 0.00E+00 | 0.00E+00 |
| Gpr171 | 6 | 10 | 0.847 | 0.076 | 1.48E+00 | 0.00E+00 | 0.00E+00 |
| Emp3 | 6 | 10 | 0.897 | 0.071 | 1.43E+00 | 0.00E+00 | 0.00E+00 |
| P2ry14 | 6 | 10 | 0.874 | 0.005 | 1.40E+00 | 0.00E+00 | 0.00E+00 |
| Ramp1 | 6 | 10 | 0.879 | 0.019 | 1.33E+00 | 0.00E+00 | 0.00E+00 |
| Smim5 | 6 | 10 | 0.781 | 0.001 | 1.32E+00 | 0.00E+00 | 0.00E+00 |
| Pltp | 6 | 10 | 0.842 | 0.012 | 1.31E+00 | 0.00E+00 | 0.00E+00 |
| Upb1 | 6 | 10 | 0.86 | 0.001 | 1.29E+00 | 0.00E+00 | 0.00E+00 |
| Ccr2 | 6 | 10 | 0.777 | 0.002 | 1.26E+00 | 0.00E+00 | 0.00E+00 |
| Ifnar2 | 6 | 10 | 0.868 | 0.015 | 1.25E+00 | 0.00E+00 | 0.00E+00 |
| Ppfia4 | 6 | 10 | 0.864 | 0.006 | 1.24E+00 | 0.00E+00 | 0.00E+00 |
| Nucb2 | 6 | 10 | 0.868 | 0.034 | 1.23E+00 | 0.00E+00 | 0.00E+00 |
| S100a11 | 6 | 10 | 0.877 | 0.09 | 1.22E+00 | 0.00E+00 | 0.00E+00 |
| Runx2 | 6 | 10 | 0.811 | 0.005 | 1.19E+00 | 0.00E+00 | 0.00E+00 |
| Cadm1 | 6 | 10 | 0.848 | 0.001 | 1.18E+00 | 0.00E+00 | 0.00E+00 |
| Lgals3 | 6 | 10 | 0.756 | 0.03 | 1.17E+00 | 0.00E+00 | 0.00E+00 |
| Cd68 | 6 | 10 | 0.83 | 0.019 | 1.17E+00 | 0.00E+00 | 0.00E+00 |
| Kctd12 | 6 | 10 | 0.81 | 0.017 | 1.15E+00 | 0.00E+00 | 0.00E+00 |
| Snx18 | 6 | 10 | 0.81 | 0.046 | 1.11E+00 | 0.00E+00 | 0.00E+00 |
| Amica1 | 6 | 10 | 0.791 | 0.009 | 1.11E+00 | 0.00E+00 | 0.00E+00 |
| Evi2a | 6 | 10 | 0.768 | 0.076 | 1.05E+00 | 0.00E+00 | 0.00E+00 |
| Trib1 | 6 | 10 | 0.722 | 0.046 | 1.03E+00 | 0.00E+00 | 0.00E+00 |
| Pir | 6 | 10 | 0.67 | 0.002 | 1.01E+00 | 0.00E+00 | 0.00E+00 |
| Il7r | 6 | 10 | 0.757 | 0.026 | 1.00E+00 | 0.00E+00 | 0.00E+00 |
| Zeb2 | 6 | 10 | 0.725 | 0.015 | 9.84E−01 | 0.00E+00 | 0.00E+00 |
| Tubgcp5 | 6 | 10 | 0.744 | 0.051 | 9.72E−01 | 0.00E+00 | 0.00E+00 |
| Sh3bgr | 6 | 10 | 0.7 | 0.004 | 9.55E−01 | 0.00E+00 | 0.00E+00 |
| Kmo | 6 | 10 | 0.79 | 0.075 | 9.52E−01 | 0.00E+00 | 0.00E+00 |
| Litaf | 6 | 10 | 0.728 | 0.049 | 9.47E−01 | 0.00E+00 | 0.00E+00 |
| Mndal | 6 | 10 | 0.694 | 0.024 | 9.27E−01 | 0.00E+00 | 0.00E+00 |
| Ahnak | 6 | 10 | 0.688 | 0.018 | 9.02E−01 | 0.00E+00 | 0.00E+00 |
| Klrd1 | 6 | 10 | 0.685 | 0.007 | 8.85E−01 | 0.00E+00 | 0.00E+00 |
| Timp2 | 6 | 10 | 0.68 | 0.014 | 8.66E−01 | 0.00E+00 | 0.00E+00 |
| Sla2 | 6 | 10 | 0.67 | 0.016 | 8.43E−01 | 0.00E+00 | 0.00E+00 |
| Atp2a1 | 6 | 10 | 0.654 | 0.001 | 8.17E−01 | 0.00E+00 | 0.00E+00 |
| I830077J02Rik | 6 | 10 | 0.63 | 0.002 | 8.07E−01 | 0.00E+00 | 0.00E+00 |
| Tbc1d8 | 6 | 10 | 0.593 | 0.001 | 7.06E−01 | 3.7E−313 | 6.72E−306 |
| Cd209a | 6 | 10 | 0.578 | 0.002 | 1.25E+00 | 5.29E−306 | 9.37E−299 |
| Slamf9 | 6 | 10 | 0.592 | 0.004 | 7.98E−01 | 7.25E−302 | 1.28E−294 |
| Sdc4 | 6 | 10 | 0.676 | 0.044 | 8.31E−01 | 3.09E−300 | 5.47E−293 |
| Klra17 | 6 | 10 | 0.57 | 0.001 | 7.71E−01 | 6.75E−298 | 1.20E−290 |
| Tmem229b | 6 | 10 | 0.706 | 0.059 | 8.33E−01 | 1.66E−295 | 2.94E−288 |
| Cd209d | 6 | 10 | 0.564 | 0.006 | 1.29E+00 | 1.24E−287 | 2.20E−280 |
| Csf2rb | 6 | 10 | 0.577 | 0.005 | 6.92E−01 | 4.51E−283 | 7.98E−276 |
| Plaur | 6 | 10 | 0.561 | 0.006 | 1.01E+00 | 5.25E−282 | 9.31E−275 |
| Rell1 | 6 | 10 | 0.586 | 0.01 | 7.35E−01 | 6.41E−281 | 1.14E−273 |
| Irf2bp2 | 6 | 10 | 0.712 | 0.073 | 8.55E−01 | 4.60E−278 | 8.14E−271 |
| Zyx | 6 | 10 | 0.623 | 0.024 | 7.76E−01 | 6.37E−273 | 1.13E−265 |
| Cd8b1 | 6 | 10 | 0.593 | 0.027 | 1.78E+00 | 9.61E−270 | 1.70E−262 |
| Lifr | 6 | 10 | 0.543 | 0.005 | 6.46E−01 | 2.42E−266 | 4.28E−259 |
| Tex2 | 6 | 10 | 0.522 | 0.002 | 5.78E−01 | 1.30E−261 | 2.30E−254 |
| Adgre5 | 6 | 10 | 0.617 | 0.03 | 7.77E−01 | 3.51E−260 | 6.22E−253 |
| Lag3 | 6 | 10 | 0.548 | 0.007 | 7.10E−01 | 1.09E−255 | 1.93E−248 |
| Spns3 | 6 | 10 | 0.564 | 0.011 | 6.66E−01 | 2.50E−254 | 4.43E−247 |
| Ifi203 | 6 | 10 | 0.573 | 0.023 | 7.73E−01 | 9.81E−254 | 1.74E−246 |
| Klrk1 | 6 | 10 | 0.522 | 0.005 | 7.03E−01 | 1.72E−251 | 3.05E−244 |
| Irf7 | 6 | 10 | 0.712 | 0.1 | 1.04E+00 | 5.55E−251 | 9.83E−244 |
| Il10rb | 6 | 10 | 0.59 | 0.026 | 6.48E−01 | 2.77E−249 | 4.90E−242 |
| Leprotl1 | 6 | 10 | 0.634 | 0.045 | 6.91E−01 | 8.56E−245 | 1.52E−237 |
| Trf | 6 | 10 | 0.513 | 0.006 | 6.20E−01 | 1.30E−239 | 2.30E−232 |
| Irf1 | 6 | 10 | 0.65 | 0.065 | 8.30E−01 | 3.67E−239 | 6.51E−232 |
| Rnf149 | 6 | 10 | 0.601 | 0.041 | 8.20E−01 | 3.18E−228 | 5.63E−221 |
| Trim30a | 6 | 10 | 0.597 | 0.045 | 7.07E−01 | 1.57E−226 | 2.78E−219 |
| Fgr | 6 | 10 | 0.623 | 0.055 | 6.93E−01 | 1.46E−225 | 2.59E−218 |
| Gpr183 | 6 | 10 | 0.624 | 0.048 | 8.64E−01 | 1.35E−223 | 2.38E−216 |
| Itgb7 | 6 | 10 | 0.622 | 0.058 | 7.28E−01 | 3.41E−217 | 6.04E−210 |
| Socs3 | 6 | 10 | 0.52 | 0.019 | 9.44E−01 | 2.02E−212 | 3.58E−205 |
| Mctp2 | 6 | 10 | 0.5 | 0.02 | 5.67E−01 | 6.79E−202 | 1.20E−194 |
| Runx3 | 6 | 10 | 0.567 | 0.049 | 6.86E−01 | 2.44E−196 | 4.33E−189 |
| Cmah | 6 | 10 | 0.546 | 0.038 | 5.94E−01 | 3.91E−195 | 6.93E−188 |
| Plekhm3 | 6 | 10 | 0.635 | 0.097 | 6.60E−01 | 3.70E−191 | 6.55E−184 |
| Hhex | 6 | 10 | 0.553 | 0.048 | 6.41E−01 | 3.86E−186 | 6.83E−179 |
| Rnf13 | 6 | 10 | 0.633 | 0.101 | 6.50E−01 | 2.27E−184 | 4.02E−177 |
| Cd44 | 6 | 10 | 0.609 | 0.085 | 6.75E−01 | 3.87E−179 | 6.84E−172 |
| Nek6 | 6 | 10 | 0.518 | 0.037 | 5.74E−01 | 1.56E−174 | 2.77E−167 |
| Man1a2 | 6 | 10 | 0.548 | 0.063 | 5.81E−01 | 6.02E−172 | 1.06E−164 |
| Gpr137b | 6 | 10 | 0.53 | 0.053 | 5.28E−01 | 3.43E−170 | 6.07E−163 |
| Bhlhe40 | 6 | 10 | 0.504 | 0.04 | 6.37E−01 | 3.39E−163 | 6.00E−156 |
| Rnf122 | 6 | 10 | 0.525 | 0.064 | 5.80E−01 | 3.97E−159 | 7.02E−152 |
| Ly6c2 | 6 | 11 | 0.997 | 0.069 | 3.10E+00 | 0.00E+00 | 0.00E+00 |
| Siglech | 6 | 11 | 0.998 | 0.093 | 2.69E+00 | 0.00E+00 | 0.00E+00 |
| Ly6c1 | 6 | 11 | 0.991 | 0.028 | 2.59E+00 | 0.00E+00 | 0.00E+00 |
| Cox6a2 | 6 | 11 | 0.969 | 0.037 | 2.45E+00 | 0.00E+00 | 0.00E+00 |
| Iglc3 | 6 | 11 | 0.968 | 0.024 | 2.38E+00 | 0.00E+00 | 0.00E+00 |
| Rnase6 | 6 | 11 | 0.992 | 0.031 | 2.33E+00 | 0.00E+00 | 0.00E+00 |
| Ctsh | 6 | 11 | 0.988 | 0.07 | 2.23E+00 | 0.00E+00 | 0.00E+00 |
| Mpeg1 | 6 | 11 | 0.983 | 0.048 | 2.05E+00 | 0.00E+00 | 0.00E+00 |
| Cybb | 6 | 11 | 0.978 | 0.028 | 1.95E+00 | 0.00E+00 | 0.00E+00 |
| Klk1b27 | 6 | 11 | 0.965 | 0.011 | 1.91E+00 | 5.08E−307 | 9.00E−300 |
| Pld4 | 6 | 11 | 0.975 | 0.053 | 1.90E+00 | 4.35E−294 | 7.70E−287 |
| Klk1 | 6 | 11 | 0.96 | 0.066 | 2.62E+00 | 6.06E−290 | 1.07E−282 |
| Ctsl | 6 | 11 | 0.96 | 0.046 | 2.11E+00 | 5.69E−268 | 1.01E−260 |
| Sell | 6 | 11 | 0.973 | 0.094 | 1.77E+00 | 1.66E−265 | 2.95E−258 |
| Lair1 | 6 | 11 | 0.937 | 0.021 | 1.57E+00 | 1.26E−262 | 2.23E−255 |
| Mef2c | 6 | 11 | 0.947 | 0.048 | 1.59E+00 | 1.86E−250 | 3.30E−243 |
| Clec12a | 6 | 11 | 0.93 | 0.025 | 1.63E+00 | 1.14E−244 | 2.02E−237 |
| D13Ertd608e | 6 | 11 | 0.924 | 0.007 | 1.94E+00 | 7.09E−243 | 1.26E−235 |
| Alox5ap | 6 | 11 | 0.937 | 0.031 | 1.55E+00 | 9.83E−238 | 1.74E−230 |
| Bcl11a | 6 | 11 | 0.889 | 0.018 | 1.43E+00 | 5.00E−214 | 8.85E−207 |
| Ppfia4 | 6 | 11 | 0.864 | 0.014 | 1.23E+00 | 3.73E−208 | 6.60E−201 |
| Tsc22d1 | 6 | 11 | 0.908 | 0.075 | 1.60E+00 | 2.65E−201 | 4.69E−194 |
| Nucb2 | 6 | 11 | 0.868 | 0.01 | 1.25E+00 | 1.19E−199 | 2.11E−192 |
| P2ry14 | 6 | 11 | 0.874 | 0.024 | 1.38E+00 | 2.54E−193 | 4.50E−186 |
| Cadm1 | 6 | 11 | 0.848 | 0.012 | 1.17E+00 | 3.48E−192 | 6.15E−185 |
| Upb1 | 6 | 11 | 0.86 | 0.015 | 1.28E+00 | 4.89E−185 | 8.66E−178 |
| Ifi27l2a | 6 | 11 | 0.844 | 0.07 | 2.10E+00 | 6.88E−179 | 1.22E−171 |
| Spib | 6 | 11 | 0.823 | 0.022 | 1.21E+00 | 7.33E−177 | 1.30E−169 |
| Smim5 | 6 | 11 | 0.781 | 0.012 | 1.31E+00 | 2.16E−174 | 3.82E−167 |
| Cd68 | 6 | 11 | 0.83 | 0.016 | 1.17E+00 | 5.67E−160 | l.OOE−152 |
| Slpi | 6 | 11 | 0.789 | 0.024 | 1.67E+00 | 4.23E−159 | 7.48E−152 |
| Ly6c2 | 6 | 12 | 0.997 | 0.048 | 3.15E+00 | 0.00E+00 | 0.00E+00 |
| Ly6c1 | 6 | 12 | 0.991 | 0.017 | 2.61E+00 | 0.00E+00 | 0.00E+00 |
| Cox6a2 | 6 | 12 | 0.969 | 0.027 | 2.46E+00 | 0.00E+00 | 0.00E+00 |
| Iglc3 | 6 | 12 | 0.968 | 0.019 | 2.38E+00 | 0.00E+00 | 0.00E+00 |
| Rnase6 | 6 | 12 | 0.992 | 0.032 | 2.33E+00 | 0.00E+00 | 0.00E+00 |
| Ctsh | 6 | 12 | 0.988 | 0.098 | 2.20E+00 | 0.00E+00 | 0.00E+00 |
| Mpeg1 | 6 | 12 | 0.983 | 0.036 | 2.06E+00 | 0.00E+00 | 0.00E+00 |
| Klk1b27 | 6 | 12 | 0.965 | 0.018 | 1.90E+00 | 5.0E−315 | 8.89E−308 |
| Pld4 | 6 | 12 | 0.975 | 0.061 | 1.90E+00 | 1.48E−307 | 2.62E−300 |
| Tcf4 | 6 | 12 | 0.977 | 0.096 | 1.84E+00 | 1.50E−304 | 2.65E−297 |
| Klk1 | 6 | 12 | 0.96 | 0.072 | 2.61E+00 | 4.78E−300 | 8.46E−293 |
| Lgals1 | 6 | 12 | 0.978 | 0.085 | 2.13E+00 | 7.50E−297 | 1.33E−289 |
| Sell | 6 | 12 | 0.973 | 0.081 | 1.79E+00 | 9.66E−291 | 1.71E−283 |
| Mef2c | 6 | 12 | 0.947 | 0.021 | 1.62E+00 | 1.14E−287 | 2.02E−280 |
| Lair1 | 6 | 12 | 0.937 | 0.023 | 1.57E+00 | 2.43E−274 | 4.30E−267 |
| Ctsl | 6 | 12 | 0.96 | 0.067 | 2.09E+00 | 5.61E−273 | 9.93E−266 |
| Clec12a | 6 | 12 | 0.93 | 0.017 | 1.64E+00 | 2.53E−265 | 4.47E−258 |
| D13Ertd608e | 6 | 12 | 0.924 | 0.007 | 1.94E+00 | 5.62E−257 | 9.96E−250 |
| Alox5ap | 6 | 12 | 0.937 | 0.032 | 1.55E+00 | 1.67E−253 | 2.95E−246 |
| Ctss | 6 | 12 | 0.96 | 0.087 | 1.71E+00 | 1.39E−238 | 2.46E−231 |
| Bcl11a | 6 | 12 | 0.889 | 0.023 | 1.43E+00 | 1.66E−217 | 2.95E−210 |
| Atp1b1 | 6 | 12 | 0.897 | 0.033 | 1.48E+00 | 8.29E−213 | 1.47E−205 |
| Ppfia4 | 6 | 12 | 0.864 | 0.017 | 1.23E+00 | 3.85E−212 | 6.82E−205 |
| Cadm1 | 6 | 12 | 0.848 | 0.011 | 1.17E+00 | 3.92E−202 | 6.93E−195 |
| Ifi27l2a | 6 | 12 | 0.844 | 0.05 | 2.14E+00 | 5.33E−199 | 9.43E−192 |
| Tsc22d1 | 6 | 12 | 0.908 | 0.099 | 1.55E+00 | 7.63E−195 | 1.35E−187 |
| Nucb2 | 6 | 12 | 0.868 | 0.024 | 1.24E+00 | 2.42E−192 | 4.29E−185 |
| Spib | 6 | 12 | 0.823 | 0.021 | 1.21E+00 | 5.83E−184 | 1.03E−176 |
| Upb1 | 6 | 12 | 0.86 | 0.026 | 1.27E+00 | 4.32E−182 | 7.65E−175 |
| Ly6e | 6 | 12 | 0.891 | 0.088 | 1.85E+00 | 9.81E−178 | 1.74E−170 |
| Smim5 | 6 | 12 | 0.781 | 0.018 | 1.30E+00 | 5.63E−173 | 9.97E−166 |
| Slpi | 6 | 12 | 0.789 | 0.024 | 1.67E+00 | 4.60E−164 | 8.15E−157 |
| Ncf1 | 6 | 12 | 0.861 | 0.072 | 1.38E+00 | 1.18E−163 | 2.08E−156 |
| Cd68 | 6 | 12 | 0.83 | 0.024 | 1.16E+00 | 1.24E−160 | 2.19E−153 |
| Scimp | 6 | 12 | 0.785 | 0.008 | 1.07E+00 | 3.82E−159 | 6.75E−152 |
| Pkib | 6 | 12 | 0.782 | 0.019 | 1.14E+00 | 4.90E−158 | 8.67E−151 |
| Ly6c2 | 6 | 14 | 0.997 | 0.094 | 3.04E+00 | 0.00E+00 | 0.00E+00 |
| Ly6c1 | 6 | 14 | 0.991 | 0.041 | 2.56E+00 | 0.00E+00 | 0.00E+00 |
| Cox6a2 | 6 | 14 | 0.969 | 0.049 | 2.44E+00 | 0.00E+00 | 0.00E+00 |
| Iglc3 | 6 | 14 | 0.968 | 0.092 | 2.23E+00 | 0.00E+00 | 0.00E+00 |
| D13Ertd608e | 6 | 14 | 0.924 | 0.014 | 1.93E+00 | 0.00E+00 | 0.00E+00 |
| P2ry14 | 6 | 14 | 0.874 | 0.059 | 1.34E+00 | 0.00E+00 | 0.00E+00 |
| Nucb2 | 6 | 14 | 0.868 | 0.08 | 1.17E+00 | 0.00E+00 | 0.00E+00 |
| Mzb1 | 6 | 14 | 0.81 | 0.094 | 1.23E+00 | 2.0E−316 | 0.00E+00 |
| Slpi | 6 | 14 | 0.789 | 0.056 | 1.63E+00 | 3.1E−316 | 0.00E+00 |
| Lefty1 | 6 | 14 | 0.739 | 0.033 | 9.62E−01 | 2.90E−292 | 5.14E−285 |
| Ccr2 | 6 | 14 | 0.777 | 0.055 | 1.15E+00 | 8.71E−281 | 1.54E−273 |
| Sh3bgr | 6 | 14 | 0.7 | 0.022 | 9.38E−01 | 6.49E−278 | 1.15E−270 |
| Pir | 6 | 14 | 0.67 | 0.02 | 9.80E−01 | 3.81E−261 | 6.75E−254 |
| Atp2a1 | 6 | 14 | 0.654 | 0.019 | 7.98E−01 | 9.82E−251 | 1.74E−243 |
| Tubgcp5 | 6 | 14 | 0.744 | 0.095 | 9.24E−01 | 2.08E−249 | 3.69E−242 |
| Hpse | 6 | 14 | 0.73 | 0.099 | 8.88E−01 | 7.92E−242 | 1.40E−234 |
| Timp2 | 6 | 14 | 0.68 | 0.049 | 8.30E−01 | 3.17E−239 | 5.61E−232 |
| Havcr1 | 6 | 14 | 0.568 | 0.009 | 6.29E−01 | 2.65E−209 | 4.68E−202 |
| Ctse | 6 | 14 | 0.673 | 0.091 | 7.43E−01 | 1.66E−203 | 2.94E−196 |
| Klra17 | 6 | 14 | 0.57 | 0.013 | 7.54E−01 | 4.42E−203 | 7.83E−196 |
| Sla2 | 6 | 14 | 0.67 | 0.085 | 7.65E−01 | 1.99E−198 | 3.53E−191 |
| Slamf9 | 6 | 14 | 0.592 | 0.05 | 7.44E−01 | 1.63E−188 | 2.89E−181 |
| Cd209d | 6 | 14 | 0.564 | 0.02 | 1.28E+00 | 3.15E−188 | 5.57E−181 |
| Cd8b1 | 6 | 14 | 0.593 | 0.061 | 1.73E+00 | 2.83E−181 | 5.01E−174 |
| Lifr | 6 | 14 | 0.543 | 0.025 | 6.26E−01 | 3.74E−175 | 6.63E−168 |
| Cmah | 6 | 14 | 0.546 | 0.033 | 6.01E−01 | 6.25E−171 | 1.11E−163 |
| Plac8 | 6 | 15 | 0.999 | 0.101 | 3.39E+00 | 1.33E−188 | 2.35E−181 |
| Siglech | 6 | 17 | 0.998 | 0.053 | 2.74E+00 | 0.00E+00 | 0.00E+00 |
| Ly6c2 | 6 | 17 | 0.997 | 0.038 | 3.13E+00 | 7.09E−290 | 1.25E−282 |
| Rnase6 | 6 | 17 | 0.992 | 0.032 | 2.33E+00 | 1.18E−267 | 2.10E−260 |
| Ly6c1 | 6 | 17 | 0.991 | 0.009 | 2.61E+00 | 2.52E−265 | 4.47E−258 |
| Ctsh | 6 | 17 | 0.988 | 0.044 | 2.26E+00 | 9.57E−255 | 1.69E−247 |
| Mpeg1 | 6 | 17 | 0.983 | 0.018 | 2.08E+00 | 1.59E−245 | 2.81E−238 |
| Cybb | 6 | 17 | 0.978 | 0.023 | 1.95E+00 | 1.45E−229 | 2.57E−222 |
| Cox6a2 | 6 | 17 | 0.969 | 0.015 | 2.47E+00 | 8.65E−223 | 1.53E−215 |
| Lgals1 | 6 | 17 | 0.978 | 0.065 | 2.24E+00 | 1.20E−213 | 2.12E−206 |
| Iglc3 | 6 | 17 | 0.968 | 0.035 | 2.36E+00 | 1.61E−211 | 2.85E−204 |
| Pld4 | 6 | 17 | 0.975 | 0.038 | 1.92E+00 | 3.79E−211 | 6.71E−204 |
| Klk1b27 | 6 | 17 | 0.965 | 0.015 | 1.90E+00 | 1.19E−204 | 2.10E−197 |
| Sell | 6 | 17 | 0.973 | 0.062 | 1.80E+00 | 1.53E−198 | 2.70E−191 |
| Klk1 | 6 | 17 | 0.96 | 0.059 | 2.63E+00 | 4.21E−192 | 7.45E−185 |
| Ctsl | 6 | 17 | 0.96 | 0.032 | 2.13E+00 | 2.12E−188 | 3.74E−181 |
| Clec12a | 6 | 17 | 0.93 | 0 | 1.66E+00 | 3.48E−180 | 6.16E−173 |
| Alox5ap | 6 | 17 | 0.937 | 0.006 | 1.58E+00 | 3.24E−177 | 5.73E−170 |
| Mef2c | 6 | 17 | 0.947 | 0.041 | 1.59E+00 | 9.11E−172 | 1.61E−164 |
| Lair1 | 6 | 17 | 0.937 | 0.026 | 1.57E+00 | 6.35E−171 | 1.12E−163 |
| D13Ertd608e | 6 | 17 | 0.924 | 0.003 | 1.94E+00 | 1.23E−163 | 2.17E−156 |
| Tyrobp | 6 | 18 | 0.999 | 0.06 | 3.68E+00 | 0.00E+00 | 0.00E+00 |
| Plac8 | 6 | 18 | 0.999 | 0.06 | 3.43E+00 | 0.00E+00 | 0.00E+00 |
| Siglech | 6 | 18 | 0.998 | 0.011 | 2.78E+00 | 1.41E−259 | 2.49E−252 |
| Ly6c2 | 6 | 18 | 0.997 | 0.064 | 3.07E+00 | 1.48E−245 | 2.62E−238 |
| Rnase6 | 6 | 18 | 0.992 | 0.007 | 2.36E+00 | 1.88E−233 | 3.34E−226 |
| Ctsh | 6 | 18 | 0.988 | 0.021 | 2.29E+00 | 1.17E−220 | 2.07E−213 |
| Ly6c1 | 6 | 18 | 0.991 | 0.039 | 2.56E+00 | 1.84E−214 | 3.25E−207 |
| Mpeg1 | 6 | 18 | 0.983 | 0.007 | 2.09E+00 | 2.41E−212 | 4.26E−205 |
| Grn | 6 | 18 | 0.986 | 0.046 | 2.27E+00 | 8.35E−202 | 1.48E−194 |
| Tcf4 | 6 | 18 | 0.977 | 0.011 | 1.93E+00 | 1.24E−196 | 2.20E−189 |
| Cybb | 6 | 18 | 0.978 | 0.025 | 1.96E+00 | 2.11E−192 | 3.73E−185 |
| Pld4 | 6 | 18 | 0.975 | 0.004 | 1.95E+00 | 1.18E−191 | 2.10E−184 |
| Cox6a2 | 6 | 18 | 0.969 | 0.004 | 2.49E+00 | 4.26E−191 | 7.54E−184 |
| Klk1b27 | 6 | 18 | 0.965 | 0.004 | 1.92E+00 | 6.06E−177 | 1.07E−169 |
| Iglc3 | 6 | 18 | 0.968 | 0.053 | 2.35E+00 | 2.30E−174 | 4.08E−167 |
| Klk1 | 6 | 18 | 0.96 | 0.018 | 2.67E+00 | 7.26E−171 | 1.29E−163 |
| Siglech | 6 | 19 | 0.998 | 0.048 | 2.74E+00 | 6.67E−163 | 1.18E−155 |
| Mpeg1 | 6 | 20 | 0.983 | 0.076 | 2.01E+00 | 2.84E−257 | 5.03E−250 |
| Cox6a2 | 6 | 20 | 0.969 | 0.068 | 2.42E+00 | 7.10E−237 | 1.26E−229 |
| Klk1b27 | 6 | 20 | 0.965 | 0.059 | 1.86E+00 | 2.63E−218 | 4.66E−211 |
| D13Ertd608e | 6 | 20 | 0.924 | 0.013 | 1.93E+00 | 1.81E−197 | 3.21E−190 |
| Lair1 | 6 | 20 | 0.937 | 0.034 | 1.56E+00 | 2.85E−195 | 5.05E−188 |
| Alox5ap | 6 | 20 | 0.937 | 0.068 | 1.50E+00 | 5.83E−181 | 1.03E−173 |
| Clec12a | 6 | 20 | 0.93 | 0.064 | 1.59E+00 | 2.00E−178 | 3.54E−171 |
| Fyb | 6 | 20 | 0.944 | 0.097 | 1.45E+00 | 1.49E−171 | 2.64E−164 |
| Cox6a2 | 6 | 21 | 0.969 | 0.064 | 2.41E+00 | 1.26E−286 | 2.23E−279 |
| Iglc3 | 6 | 21 | 0.968 | 0.081 | 2.31E+00 | 1.72E−274 | 3.05E−267 |
| D13Ertd608e | 6 | 21 | 0.924 | 0.021 | 1.92E+00 | 3.39E−254 | 6.01E−247 |
| Atp1b1 | 6 | 21 | 0.897 | 0.081 | 1.42E+00 | 2.06E−204 | 3.64E−197 |
| Pltp | 6 | 21 | 0.842 | 0.094 | 1.17E+00 | 5.48E−176 | 9.70E−169 |
| Pacsin1 | 6 | 21 | 0.819 | 0.06 | 1.04E+00 | 5.47E−173 | 9.69E−166 |
| Cox6a2 | 6 | 22 | 0.969 | 0.039 | 2.45E+00 | 1.15E−237 | 2.04E−230 |
| Klk1b27 | 6 | 22 | 0.965 | 0.017 | 1.90E+00 | 2.03E−237 | 3.59E−230 |
| Iglc3 | 6 | 22 | 0.968 | 0.069 | 2.30E+00 | 2.07E−228 | 3.67E−221 |
| Ccr9 | 6 | 22 | 0.956 | 0.06 | 1.86E+00 | 7.64E−206 | 1.35E−198 |
| D13Ertd608e | 6 | 22 | 0.924 | 0.004 | 1.94E+00 | 1.52E−198 | 2.70E−191 |
| Siglech | 6 | 24 | 0.998 | 0.085 | 2.66E+00 | 0.00E+00 | 0.00E+00 |
| Ly6c2 | 6 | 24 | 0.997 | 0.072 | 2.99E+00 | 2.84E−299 | 5.03E−292 |
| Ly6c1 | 6 | 24 | 0.991 | 0.013 | 2.57E+00 | 1.26E−280 | 2.23E−273 |
| Mpeg1 | 6 | 24 | 0.983 | 0.067 | 1.94E+00 | 4.29E−240 | 7.60E−233 |
| Cox6a2 | 6 | 24 | 0.969 | 0.031 | 2.42E+00 | 7.94E−234 | 1.41E−226 |
| Sell | 6 | 24 | 0.973 | 0.063 | 1.80E+00 | 2.73E−224 | 4.84E−217 |
| Iglc3 | 6 | 24 | 0.968 | 0.081 | 2.27E+00 | 2.72E−220 | 4.81E−213 |
| Klk1b27 | 6 | 24 | 0.965 | 0.054 | 1.86E+00 | 3.66E−216 | 6.49E−209 |
| D13Ertd608e | 6 | 24 | 0.924 | 0.004 | 1.93E+00 | 1.09E−194 | 1.93E−187 |
| Clec12a | 6 | 24 | 0.93 | 0.036 | 1.57E+00 | 5.33E−180 | 9.44E−173 |
| Rnase6 | 6 | 25 | 0.992 | 0.023 | 2.34E+00 | 6.71E−176 | 1.19E−168 |
| Ighm | 6 | 25 | 0.993 | 0.077 | 2.22E+00 | 4.96E−173 | 8.78E−166 |
| Ly6c2 | 6 | 26 | 0.997 | 0.074 | 3.02E+00 | 1.89E−279 | 3.35E−272 |
| Ly6c1 | 6 | 26 | 0.991 | 0.014 | 2.58E+00 | 1.16E−259 | 2.05E−252 |
| Mpeg1 | 6 | 26 | 0.983 | 0.065 | 2.01E+00 | 7.29E−228 | 1.29E−220 |
| Cybb | 6 | 26 | 0.978 | 0.055 | 1.89E+00 | 5.08E−215 | 8.99E−208 |
| Pld4 | 6 | 26 | 0.975 | 0.083 | 1.84E+00 | 3.94E−209 | 6.97E−202 |
| Cox6a2 | 6 | 26 | 0.969 | 0.055 | 2.38E+00 | 6.24E−207 | 1.10E−199 |
| Tcf4 | 6 | 26 | 0.977 | 0.097 | 1.81E+00 | 1.15E−206 | 2.03E−199 |
| Iglc3 | 6 | 26 | 0.968 | 0.069 | 2.29E+00 | 1.39E−204 | 2.45E−197 |
| Klk1b27 | 6 | 26 | 0.965 | 0.065 | 1.83E+00 | 3.67E−190 | 6.49E−183 |
| Ctss | 6 | 26 | 0.96 | 0.088 | 1.68E+00 | 2.68E−184 | 4.75E−177 |
| Mef2c | 6 | 26 | 0.947 | 0.037 | 1.59E+00 | 2.52E−178 | 4.46E−171 |
| Ccr9 | 6 | 26 | 0.956 | 0.065 | 1.83E+00 | 1.24E−177 | 2.19E−170 |
| D13Ertd608e | 6 | 26 | 0.924 | 0.014 | 1.90E+00 | 2.13E−166 | 3.77E−159 |
| Clec12a | 6 | 26 | 0.93 | 0.046 | 1.60E+00 | 9.21E−160 | 1.63E−152 |
| Siglech | 6 | 29 | 0.998 | 0.014 | 2.74E+00 | 9.57E−222 | 1.69E−214 |
| Ly6c2 | 6 | 29 | 0.997 | 0.101 | 2.92E+00 | 1.64E−203 | 2.90E−196 |
| Grn | 6 | 29 | 0.986 | 0.095 | 2.18E+00 | 5.73E−184 | 1.02E−176 |
| Ly6c1 | 6 | 29 | 0.991 | 0.074 | 2.45E+00 | 2.29E−183 | 4.05E−176 |
| Mpeg1 | 6 | 29 | 0.983 | 0.047 | 2.03E+00 | 4.62E−183 | 8.17E−176 |
| Cox6a2 | 6 | 29 | 0.969 | 0.014 | 2.48E+00 | 8.33E−165 | 1.47E−157 |
| Klk1b27 | 6 | 29 | 0.965 | 0.014 | 1.91E+00 | 2.04E−160 | 3.61E−153 |
| Siglech | 6 | 30 | 0.998 | 0.067 | 2.69E+00 | 2.05E−162 | 3.62E−155 |
| Siglech | 6 | 33 | 0.998 | 0.076 | 2.68E+00 | 6.01E−198 | 1.06E−190 |
| Ly6c2 | 6 | 33 | 0.997 | 0.019 | 3.18E+00 | 4.19E−173 | 7.41E−166 |
| Rnase6 | 6 | 35 | 0.992 | 0.071 | 2.24E+00 | 4.20E−180 | 7.44E−173 |
| Ly6c1 | 6 | 35 | 0.991 | 0.02 | 2.53E+00 | 6.21E−175 | 1.10E−167 |
| Pld4 | 6 | 35 | 0.975 | 0.101 | 1.80E+00 | 1.27E−163 | 2.24E−156 |
| Siglech | 6 | 37 | 0.998 | 0.034 | 2.76E+00 | 1.07E−186 | 1.89E−179 |
| Ctsh | 6 | 37 | 0.988 | 0.057 | 2.25E+00 | 7.37E−162 | 1.30E−154 |
| Ly6c2 | 6 | 37 | 0.997 | 0.08 | 2.93E+00 | 1.15E−160 | 2.03E−153 |
| Grn | 6 | 37 | 0.986 | 0.068 | 2.21E+00 | 4.72E−159 | 8.35E−152 |
| Rnase6 | 6 | 37 | 0.992 | 0.023 | 2.34E+00 | 8.48E−159 | 1.50E−151 |
| Izumo1r | 7 | 8 | 0.861 | 0.008 | 1.63E+00 | 0.00E+00 | 0.00E+00 |
| Ifi27l2a | 7 | 8 | 0.802 | 0.054 | 1.61E+00 | 0.00E+00 | 0.00E+00 |
| Cd3e | 7 | 8 | 0.94 | 0.016 | 1.59E+00 | 0.00E+00 | 0.00E+00 |
| Trac | 7 | 8 | 0.883 | 0.056 | 1.47E+00 | 0.00E+00 | 0.00E+00 |
| Rgs10 | 7 | 8 | 0.857 | 0.099 | 1.46E+00 | 0.00E+00 | 0.00E+00 |
| Izumo1r | 7 | 9 | 0.861 | 0.062 | 1.57E+00 | 0.00E+00 | 0.00E+00 |
| Trac | 7 | 9 | 0.883 | 0.083 | 1.45E+00 | 0.00E+00 | 0.00E+00 |
| S100a10 | 7 | 9 | 0.741 | 0.093 | 1.14E+00 | 0.00E+00 | 0.00E+00 |
| Skap1 | 7 | 9 | 0.739 | 0.088 | 8.68E−01 | 0.00E+00 | 0.00E+00 |
| Cd28 | 7 | 9 | 0.704 | 0.037 | 9.08E−01 | 3.10E−291 | 5.48E−284 |
| Cd247 | 7 | 9 | 0.56 | 0.055 | 6.59E−01 | 7.39E−279 | 1.31E−271 |
| Tcf7 | 7 | 9 | 0.567 | 0.052 | 7.85E−01 | 2.87E−250 | 5.08E−243 |
| Thy1 | 7 | 9 | 0.548 | 0.086 | 7.66E−01 | 7.64E−240 | 1.35E−232 |
| Tnfrsf18 | 7 | 9 | 0.511 | 0.052 | 6.81E−01 | 3.25E−186 | 5.74E−179 |
| Trbc1 | 7 | 10 | 0.666 | 0.064 | 1.83E+00 | 0.00E+00 | 0.00E+00 |
| Cd3g | 7 | 10 | 0.968 | 0.055 | 1.72E+00 | 0.00E+00 | 0.00E+00 |
| Izumo1r | 7 | 10 | 0.861 | 0.029 | 1.62E+00 | 0.00E+00 | 0.00E+00 |
| Cd3d | 7 | 10 | 0.94 | 0.083 | 1.54E+00 | 0.00E+00 | 0.00E+00 |
| Cd3e | 7 | 10 | 0.94 | 0.07 | 1.54E+00 | 0.00E+00 | 0.00E+00 |
| Trac | 7 | 10 | 0.883 | 0.05 | 1.49E+00 | 0.00E+00 | 0.00E+00 |
| S100a10 | 7 | 10 | 0.741 | 0.053 | 1.19E+00 | 0.00E+00 | 0.00E+00 |
| Ms4a4b | 7 | 10 | 0.695 | 0.07 | 1.19E+00 | 0.00E+00 | 0.00E+00 |
| Lat | 7 | 10 | 0.829 | 0.076 | 1.17E+00 | 0.00E+00 | 0.00E+00 |
| Fyb | 7 | 10 | 0.809 | 0.059 | 1.11E+00 | 0.00E+00 | 0.00E+00 |
| S100a11 | 7 | 10 | 0.675 | 0.09 | 9.97E−01 | 0.00E+00 | 0.00E+00 |
| Cd2 | 7 | 10 | 0.741 | 0.074 | 9.65E−01 | 0.00E+00 | 0.00E+00 |
| Hcst | 7 | 10 | 0.746 | 0.089 | 9.60E−01 | 0.00E+00 | 0.00E+00 |
| Cd28 | 7 | 10 | 0.704 | 0.013 | 9.39E−01 | 0.00E+00 | 0.00E+00 |
| Skap1 | 7 | 10 | 0.739 | 0.035 | 9.24E−01 | 0.00E+00 | 0.00E+00 |
| Tcf7 | 7 | 10 | 0.567 | 0.029 | 8.17E−01 | 0.00E+00 | 0.00E+00 |
| Thy1 | 7 | 10 | 0.548 | 0.049 | 8.12E−01 | 0.00E+00 | 0.00E+00 |
| Cd247 | 7 | 10 | 0.56 | 0.025 | 6.92E−01 | 0.00E+00 | 0.00E+00 |
| Tnfrsf18 | 7 | 10 | 0.511 | 0.024 | 7.11E−01 | 8.12E−305 | 1.44E−297 |
| Ccnd2 | 7 | 10 | 0.508 | 0.044 | 6.89E−01 | 3.76E−300 | 6.66E−293 |
| Izumo1r | 7 | 11 | 0.861 | 0.005 | 1.64E+00 | 0.00E+00 | 0.00E+00 |
| Cd3g | 7 | 11 | 0.968 | 0.069 | 1.64E+00 | 0.00E+00 | 0.00E+00 |
| Ifi27l2a | 7 | 11 | 0.802 | 0.07 | 1.60E+00 | 0.00E+00 | 0.00E+00 |
| Cd3e | 7 | 11 | 0.94 | 0.021 | 1.57E+00 | 0.00E+00 | 0.00E+00 |
| Cd3d | 7 | 11 | 0.94 | 0.047 | 1.57E+00 | 0.00E+00 | 0.00E+00 |
| Cd247 | 7 | 11 | 0.56 | 0.097 | 6.18E−01 | 6.59E−169 | 1.17E−161 |
| Slamf6 | 7 | 11 | 0.506 | 0.058 | 6.26E−01 | 2.89E−160 | 5.12E−153 |
| Ifi27l2a | 7 | 12 | 0.802 | 0.05 | 1.64E+00 | 0.00E+00 | 0.00E+00 |
| Izumo1r | 7 | 12 | 0.861 | 0.019 | 1.62E+00 | 0.00E+00 | 0.00E+00 |
| Cd3d | 7 | 12 | 0.94 | 0.028 | 1.59E+00 | 0.00E+00 | 0.00E+00 |
| Cd3e | 7 | 12 | 0.94 | 0.057 | 1.53E+00 | 0.00E+00 | 0.00E+00 |
| Trac | 7 | 12 | 0.883 | 0.014 | 1.53E+00 | 0.00E+00 | 0.00E+00 |
| Cd2 | 7 | 12 | 0.741 | 0.026 | 1.03E+00 | 0.00E+00 | 0.00E+00 |
| Ms4a4b | 7 | 12 | 0.695 | 0.051 | 1.20E+00 | 3.69E−294 | 6.54E−287 |
| Ms4a6b | 7 | 12 | 0.713 | 0.08 | 8.79E−01 | 1.14E−278 | 2.01E−271 |
| Cd247 | 7 | 12 | 0.56 | 0.027 | 6.88E−01 | 2.73E−226 | 4.83E−219 |
| Slamf6 | 7 | 12 | 0.506 | 0.023 | 6.66E−01 | 2.62E−187 | 4.64E−180 |
| Cd3g | 7 | 14 | 0.968 | 0.058 | 1.66E+00 | 0.00E+00 | 0.00E+00 |
| Cd3e | 7 | 14 | 0.94 | 0.038 | 1.53E+00 | 0.00E+00 | 0.00E+00 |
| Cd3d | 7 | 14 | 0.94 | 0.061 | 1.53E+00 | 0.00E+00 | 0.00E+00 |
| Trac | 7 | 14 | 0.883 | 0.033 | 1.48E+00 | 2.64E−306 | 4.67E−299 |
| Lat | 7 | 14 | 0.829 | 0.053 | 1.16E+00 | 1.88E−298 | 3.32E−291 |
| Izumo1r | 7 | 14 | 0.861 | 0.019 | 1.60E+00 | 6.48E−242 | 1.15E−234 |
| Skap1 | 7 | 14 | 0.739 | 0.072 | 8.69E−01 | 3.49E−239 | 6.17E−232 |
| Cd2 | 7 | 14 | 0.741 | 0.058 | 9.82E−01 | 3.58E−217 | 6.34E−210 |
| Cd28 | 7 | 14 | 0.704 | 0.03 | 9.15E−01 | 1.08E−195 | 1.92E−188 |
| Cd247 | 7 | 14 | 0.56 | 0.041 | 6.62E−01 | 9.06E−191 | 1.60E−183 |
| Izumo1r | 7 | 15 | 0.861 | 0 | 1.65E+00 | 7.11E−193 | 1.26E−185 |
| Cd3g | 7 | 15 | 0.968 | 0.04 | 1.73E+00 | 3.70E−172 | 6.55E−165 |
| Cd3d | 7 | 15 | 0.94 | 0.013 | 1.62E+00 | 2.96E−160 | 5.24E−153 |
| Izumo1r | 7 | 17 | 0.861 | 0.018 | 1.63E+00 | 5.44E−235 | 9.63E−228 |
| Cd3g | 7 | 20 | 0.968 | 0.081 | 1.64E+00 | 6.23E−254 | 1.10E−246 |
| Cd3e | 7 | 20 | 0.94 | 0.064 | 1.47E+00 | 9.72E−237 | 1.72E−229 |
| Cd3d | 7 | 20 | 0.94 | 0.093 | 1.45E+00 | 1.67E−228 | 2.96E−221 |
| Trac | 7 | 20 | 0.883 | 0.064 | 1.46E+00 | 7.54E−197 | 1.33E−189 |
| Izumo1r | 7 | 20 | 0.861 | 0.034 | 1.55E+00 | 1.71E−160 | 3.02E−153 |
| Cd3e | 7 | 21 | 0.94 | 0.064 | 1.51E+00 | 1.45E−185 | 2.56E−178 |
| Cd3d | 7 | 21 | 0.94 | 0.064 | 1.50E+00 | 1.11E−179 | 1.96E−172 |
| Lat | 7 | 21 | 0.829 | 0.098 | 1.12E+00 | 8.93E−178 | 1.58E−170 |
| Cd3g | 7 | 21 | 0.968 | 0.098 | 1.58E+00 | 1.06E−171 | 1.88E−164 |
| Cd3g | 7 | 22 | 0.968 | 0.039 | 1.73E+00 | 3.01E−248 | 5.34E−241 |
| Cd3d | 7 | 22 | 0.94 | 0.022 | 1.60E+00 | 2.68E−239 | 4.74E−232 |
| Cd3e | 7 | 22 | 0.94 | 0.039 | 1.56E+00 | 1.40E−230 | 2.48E−223 |
| Lck | 7 | 22 | 0.882 | 0.082 | 1.22E+00 | 3.03E−206 | 5.37E−199 |
| Trac | 7 | 22 | 0.883 | 0.013 | 1.53E+00 | 6.99E−202 | 1.24E−194 |
| Lat | 7 | 22 | 0.829 | 0.047 | 1.20E+00 | 7.06E−182 | 1.25E−174 |
| Izumo1r | 7 | 22 | 0.861 | 0.004 | 1.64E+00 | 2.55E−168 | 4.52E−161 |
| Trbc2 | 7 | 24 | 0.977 | 0.081 | 2.25E+00 | 3.84E−252 | 6.81E−245 |
| Cd3g | 7 | 24 | 0.968 | 0.027 | 1.69E+00 | 6.76E−247 | 1.20E−239 |
| Cd3e | 7 | 24 | 0.94 | 0.004 | 1.60E+00 | 2.10E−240 | 3.71E−233 |
| Cd3d | 7 | 24 | 0.94 | 0.018 | 1.61E+00 | 2.76E−231 | 4.89E−224 |
| Lck | 7 | 24 | 0.882 | 0.058 | 1.29E+00 | 1.39E−211 | 2.45E−204 |
| Trac | 7 | 24 | 0.883 | 0.013 | 1.52E+00 | 1.54E−195 | 2.73E−188 |
| Lat | 7 | 24 | 0.829 | 0.027 | 1.22E+00 | 8.60E−185 | 1.52E−177 |
| Izumo1r | 7 | 24 | 0.861 | 0.013 | 1.63E+00 | 3.74E−160 | 6.63E−153 |
| Trbc2 | 7 | 25 | 0.977 | 0.063 | 2.30E+00 | 5.05E−261 | 8.93E−254 |
| Cd3g | 7 | 25 | 0.968 | 0.027 | 1.75E+00 | 3.98E−246 | 7.05E−239 |
| Cd3e | 7 | 25 | 0.94 | 0.009 | 1.60E+00 | 7.30E−229 | 1.29E−221 |
| Cd3d | 7 | 25 | 0.94 | 0.018 | 1.61E+00 | 7.24E−224 | 1.28E−216 |
| Coro1a | 7 | 25 | 0.957 | 0.1 | 1.76E+00 | 4.86E−220 | 8.61E−213 |
| Limd2 | 7 | 25 | 0.919 | 0.1 | 1.57E+00 | 5.21E−191 | 9.22E−184 |
| Ptprcap | 7 | 25 | 0.91 | 0.063 | 1.50E+00 | 3.38E−190 | 5.98E−183 |
| Trac | 7 | 25 | 0.883 | 0.009 | 1.53E+00 | 9.75E−182 | 1.73E−174 |
| Lck | 7 | 25 | 0.882 | 0.045 | 1.28E+00 | 6.13E−180 | 1.09E−172 |
| Izumo1r | 7 | 25 | 0.861 | 0 | 1.65E+00 | 2.79E−167 | 4.94E−160 |
| Laptm5 | 7 | 25 | 0.9 | 0.1 | 1.28E+00 | 5.99E−166 | 1.06E−158 |
| Trbc2 | 7 | 26 | 0.977 | 0.083 | 2.26E+00 | 3.43E−253 | 6.06E−246 |
| Cd3g | 7 | 26 | 0.968 | 0.014 | 1.75E+00 | 3.10E−251 | 5.50E−244 |
| Cd3d | 7 | 26 | 0.94 | 0.032 | 1.59E+00 | 7.47E−233 | 1.32E−225 |
| Cd3e | 7 | 26 | 0.94 | 0.078 | 1.50E+00 | 1.33E−225 | 2.35E−218 |
| Lck | 7 | 26 | 0.882 | 0.032 | 1.31E+00 | 6.43E−206 | 1.14E−198 |
| Trac | 7 | 26 | 0.883 | 0.005 | 1.53E+00 | 3.70E−198 | 6.55E−191 |
| Izumo1r | 7 | 26 | 0.861 | 0.005 | 1.64E+00 | 2.77E−163 | 4.90E−156 |
| Trbc2 | 7 | 30 | 0.977 | 0.059 | 2.31E+00 | 2.58E−180 | 4.56E−173 |
| Cd3g | 7 | 30 | 0.968 | 0.044 | 1.73E+00 | 1.44E−168 | 2.55E−161 |
| Trbc2 | 7 | 32 | 0.977 | 0.017 | 2.38E+00 | 1.60E−167 | 2.83E−160 |
| Nkg7 | 8 | 9 | 0.983 | 0.068 | 2.28E+00 | 0.00E+00 | 0.00E+00 |
| Tyrobp | 8 | 9 | 0.989 | 0.093 | 2.27E+00 | 0.00E+00 | 0.00E+00 |
| Cd7 | 8 | 9 | 0.779 | 0.077 | 1.55E+00 | 0.00E+00 | 0.00E+00 |
| Klrd1 | 8 | 9 | 0.908 | 0.015 | 1.43E+00 | 0.00E+00 | 0.00E+00 |
| S100a10 | 8 | 9 | 0.899 | 0.093 | 1.24E+00 | 0.00E+00 | 0.00E+00 |
| Il2rb | 8 | 9 | 0.802 | 0.041 | 1.09E+00 | 0.00E+00 | 0.00E+00 |
| Klrk1 | 8 | 9 | 0.828 | 0.005 | 1.19E+00 | 2.02E−283 | 3.58E−276 |
| Ctla2a | 8 | 9 | 0.697 | 0.01 | 1.34E+00 | 6.60E−282 | 1.17E−274 |
| Fcer1g | 8 | 9 | 0.921 | 0.038 | 1.63E+00 | 3.24E−266 | 5.74E−259 |
| Satb1 | 8 | 9 | 0.607 | 0.069 | 7.14E−01 | 2.09E−236 | 3.70E−229 |
| Txk | 8 | 9 | 0.751 | 0.055 | 9.45E−01 | 1.70E−235 | 3.01E−228 |
| Nabp1 | 8 | 9 | 0.694 | 0.082 | 9.67E−01 | 5.57E−233 | 9.86E−226 |
| Klri2 | 8 | 9 | 0.542 | 0.002 | 8.86E−01 | 2.65E−232 | 4.69E−225 |
| Xcl1 | 8 | 9 | 0.532 | 0.01 | 1.15E+00 | 5.71E−226 | 1.01E−218 |
| Sh2d2a | 8 | 9 | 0.537 | 0.052 | 6.93E−01 | 3.54E−199 | 6.26E−192 |
| Dok2 | 8 | 9 | 0.648 | 0.046 | 7.46E−01 | 2.58E−198 | 4.57E−191 |
| Ptpn22 | 8 | 9 | 0.528 | 0.038 | 5.75E−01 | 5.06E−195 | 8.95E−188 |
| Bhlhe40 | 8 | 9 | 0.591 | 0.05 | 7.18E−01 | 1.55E−189 | 2.75E−182 |
| Klrb1c | 8 | 9 | 0.688 | 0.012 | 9.32E−01 | 6.00E−187 | 1.06E−179 |
| Eomes | 8 | 9 | 0.578 | 0.002 | 7.33E−01 | 1.91E−186 | 3.38E−179 |
| Klre1 | 8 | 9 | 0.703 | 0 | 9.51E−01 | 1.52E−185 | 2.70E−178 |
| Pglyrp1 | 8 | 9 | 0.51 | 0.027 | 5.95E−01 | 5.35E−177 | 9.48E−170 |
| Sell | 8 | 9 | 0.575 | 0.062 | 6.79E−01 | 2.79E−169 | 4.93E−162 |
| Skap1 | 8 | 9 | 0.554 | 0.088 | 5.45E−01 | 8.74E−169 | 1.55E−161 |
| Ccl5 | 8 | 10 | 0.993 | 0.084 | 3.97E+00 | 0.00E+00 | 0.00E+00 |
| Gzma | 8 | 10 | 0.745 | 0.05 | 2.96E+00 | 0.00E+00 | 0.00E+00 |
| Nkg7 | 8 | 10 | 0.983 | 0.045 | 2.31E+00 | 0.00E+00 | 0.00E+00 |
| Tyrobp | 8 | 10 | 0.989 | 0.064 | 2.28E+00 | 0.00E+00 | 0.00E+00 |
| Ncr1 | 8 | 10 | 0.951 | 0.009 | 1.72E+00 | 0.00E+00 | 0.00E+00 |
| Ms4a4b | 8 | 10 | 0.932 | 0.07 | 1.65E+00 | 0.00E+00 | 0.00E+00 |
| Fcer1g | 8 | 10 | 0.921 | 0.032 | 1.63E+00 | 0.00E+00 | 0.00E+00 |
| Cd7 | 8 | 10 | 0.779 | 0.043 | 1.58E+00 | 0.00E+00 | 0.00E+00 |
| Klrd1 | 8 | 10 | 0.908 | 0.007 | 1.44E+00 | 0.00E+00 | 0.00E+00 |
| Trbc1 | 8 | 10 | 0.852 | 0.064 | 1.39E+00 | 0.00E+00 | 0.00E+00 |
| Vps37b | 8 | 10 | 0.804 | 0.074 | 1.39E+00 | 0.00E+00 | 0.00E+00 |
| Selplg | 8 | 10 | 0.918 | 0.09 | 1.35E+00 | 0.00E+00 | 0.00E+00 |
| Ctla2a | 8 | 10 | 0.697 | 0.008 | 1.35E+00 | 0.00E+00 | 0.00E+00 |
| S100a10 | 8 | 10 | 0.899 | 0.053 | 1.30E+00 | 0.00E+00 | 0.00E+00 |
| Bcl2 | 8 | 10 | 0.799 | 0.05 | 1.30E+00 | 0.00E+00 | 0.00E+00 |
| Xcl1 | 8 | 10 | 0.532 | 0.002 | 1.20E+00 | 0.00E+00 | 0.00E+00 |
| Klrk1 | 8 | 10 | 0.828 | 0.005 | 1.18E+00 | 0.00E+00 | 0.00E+00 |
| Hcst | 8 | 10 | 0.861 | 0.089 | 1.17E+00 | 0.00E+00 | 0.00E+00 |
| Il2rb | 8 | 10 | 0.802 | 0.021 | 1.11E+00 | 0.00E+00 | 0.00E+00 |
| Ctsw | 8 | 10 | 0.789 | 0.018 | 1.08E+00 | 0.00E+00 | 0.00E+00 |
| Nabp1 | 8 | 10 | 0.694 | 0.04 | 1.01E+00 | 0.00E+00 | 0.00E+00 |
| Txk | 8 | 10 | 0.751 | 0.03 | 9.77E−01 | 0.00E+00 | 0.00E+00 |
| Klre1 | 8 | 10 | 0.703 | 0.001 | 9.50E−01 | 0.00E+00 | 0.00E+00 |
| Klrb1c | 8 | 10 | 0.688 | 0.004 | 9.41E−01 | 0.00E+00 | 0.00E+00 |
| Klri2 | 8 | 10 | 0.542 | 0.001 | 8.87E−01 | 0.00E+00 | 0.00E+00 |
| S100a11 | 8 | 10 | 0.706 | 0.09 | 7.72E−01 | 0.00E+00 | 0.00E+00 |
| Dok2 | 8 | 10 | 0.648 | 0.024 | 7.71E−01 | 0.00E+00 | 0.00E+00 |
| Eomes | 8 | 10 | 0.578 | 0.002 | 7.35E−01 | 0.00E+00 | 0.00E+00 |
| Emb | 8 | 10 | 0.52 | 0.057 | 7.30E−01 | 0.00E+00 | 0.00E+00 |
| Satb1 | 8 | 10 | 0.607 | 0.061 | 7.22E−01 | 0.00E+00 | 0.00E+00 |
| Skap1 | 8 | 10 | 0.554 | 0.035 | 6.01E−01 | 1.09E−305 | 1.92E−298 |
| Ptpn22 | 8 | 10 | 0.528 | 0.024 | 5.87E−01 | 1.50E−305 | 2.66E−298 |
| Serpinb9 | 8 | 10 | 0.596 | 0.011 | 1.00E+00 | 1.50E−302 | 2.65E−295 |
| Car2 | 8 | 10 | 0.578 | 0.1 | 8.79E−01 | 2.96E−302 | 5.24E−295 |
| Fasl | 8 | 10 | 0.552 | 0.002 | 6.73E−01 | 1.01E−298 | 1.79E−291 |
| Klre1 | 8 | 10 | 0.548 | 0.001 | 9.66E−01 | 3.41E−295 | 6.03E−288 |
| Sell | 8 | 10 | 0.575 | 0.042 | 7.00E−01 | 7.32E−295 | 1.30E−287 |
| Nr4a1 | 8 | 10 | 0.618 | 0.093 | 9.83E−01 | 1.89E−294 | 3.34E−287 |
| Id2 | 8 | 10 | 0.606 | 0.051 | 1.02E+00 | 1.75E−285 | 3.10E−278 |
| Ccl4 | 8 | 10 | 0.579 | 0.025 | 2.00E+00 | 1.26E−281 | 2.23E−274 |
| Pglyrp1 | 8 | 10 | 0.51 | 0.034 | 5.87E−01 | 5.32E−271 | 9.43E−264 |
| Serpinb6b | 8 | 10 | 0.598 | 0.09 | 8.01E−01 | 3.38E−256 | 5.99E−249 |
| Ccnd2 | 8 | 10 | 0.519 | 0.044 | 6.49E−01 | 1.88E−255 | 3.34E−248 |
| Ahnak | 8 | 10 | 0.519 | 0.018 | 5.86E−01 | 3.92E−238 | 6.95E−231 |
| Tnfaip3 | 8 | 10 | 0.534 | 0.07 | 6.06E−01 | 2.50E−236 | 4.42E−229 |
| H2-Q7 | 8 | 10 | 0.546 | 0.101 | 5.50E−01 | 7.70E−230 | 1.36E−222 |
| Gpr171 | 8 | 10 | 0.501 | 0.076 | 5.05E−01 | 7.53E−213 | 1.33E−205 |
| Eomes | 8 | 11 | 0.578 | 0.062 | 6.59E−01 | 3.01E−188 | 5.33E−181 |
| Sell | 8 | 11 | 0.575 | 0.094 | 6.33E−01 | 3.69E−161 | 6.53E−154 |
| Ms4a4b | 8 | 12 | 0.932 | 0.051 | 1.66E+00 | 0.00E+00 | 0.00E+00 |
| Ncr1 | 8 | 12 | 0.951 | 0.076 | 1.62E+00 | 0.00E+00 | 0.00E+00 |
| Klrd1 | 8 | 12 | 0.908 | 0.02 | 1.41E+00 | 0.00E+00 | 0.00E+00 |
| Klrk1 | 8 | 12 | 0.828 | 0.059 | 1.12E+00 | 0.00E+00 | 0.00E+00 |
| Cd2 | 8 | 12 | 0.681 | 0.026 | 8.63E−01 | 1.17E−278 | 2.07E−271 |
| Klrb1c | 8 | 12 | 0.688 | 0.035 | 9.02E−01 | 3.42E−270 | 6.05E−263 |
| Anxa2 | 8 | 12 | 0.625 | 0.038 | 7.75E−01 | 6.86E−225 | 1.21E−217 |
| Eomes | 8 | 12 | 0.578 | 0.021 | 7.15E−01 | 3.08E−219 | 5.46E−212 |
| Klri2 | 8 | 12 | 0.542 | 0.025 | 8.63E−01 | 1.06E−209 | 1.88E−202 |
| Klrc1 | 8 | 12 | 0.548 | 0.017 | 9.45E−01 | 5.87E−203 | 1.04E−195 |
| Xcl1 | 8 | 12 | 0.532 | 0.033 | 1.16E+00 | 1.02E−189 | 1.80E−182 |
| Fasl | 8 | 12 | 0.552 | 0.035 | 6.33E−01 | 4.99E−182 | 8.83E−175 |
| Pglyrp1 | 8 | 12 | 0.51 | 0.024 | 5.95E−01 | 2.59E−180 | 4.59E−173 |
| Car2 | 8 | 12 | 0.578 | 0.1 | 8.61E−01 | 1.15E−174 | 2.04E−167 |
| Sell | 8 | 12 | 0.575 | 0.081 | 6.54E−01 | 3.90E−166 | 6.90E−159 |
| Gzma | 8 | 13 | 0.745 | 0.003 | 3.01E+00 | 2.11E−279 | 3.73E−272 |
| Fcer1g | 8 | 13 | 0.921 | 0.017 | 1.66E+00 | 5.50E−219 | 9.75E−212 |
| Ncr1 | 8 | 14 | 0.951 | 0.077 | 1.61E+00 | 3.85E−245 | 6.82E−238 |
| Ctla2a | 8 | 14 | 0.697 | 0.052 | 1.27E+00 | 2.60E−192 | 4.60E−185 |
| Ctsw | 8 | 14 | 0.789 | 0.1 | 9.56E−01 | 1.88E−190 | 3.33E−183 |
| Ccl5 | 8 | 15 | 0.993 | 0.085 | 3.90E+00 | 7.57E−296 | 1.34E−288 |
| Gzma | 8 | 15 | 0.745 | 0.032 | 2.97E+00 | 3.99E−238 | 7.07E−231 |
| Gzma | 8 | 16 | 0.745 | 0.098 | 2.26E+00 | 1.41E−181 | 2.49E−174 |
| Ccl5 | 8 | 18 | 0.993 | 0.085 | 3.77E+00 | 0.00E+00 | 0.00E+00 |
| Tyrobp | 8 | 18 | 0.989 | 0.06 | 2.30E+00 | 0.00E+00 | 0.00E+00 |
| Ncr1 | 8 | 18 | 0.951 | 0.007 | 1.72E+00 | 7.58E−263 | 1.34E−255 |
| Klrd1 | 8 | 18 | 0.908 | 0.028 | 1.40E+00 | 4.84E−222 | 8.57E−215 |
| Fcer1g | 8 | 18 | 0.921 | 0.05 | 1.63E+00 | 3.56E−217 | 6.30E−210 |
| Klrk1 | 8 | 18 | 0.828 | 0.021 | 1.17E+00 | 6.13E−178 | 1.09E−170 |
| Cd7 | 8 | 18 | 0.779 | 0.05 | 1.58E+00 | 1.99E−172 | 3.52E−165 |
| Ncr1 | 8 | 20 | 0.951 | 0.089 | 1.61E+00 | 1.17E−194 | 2.08E−187 |
| Klrd1 | 8 | 20 | 0.908 | 0.093 | 1.34E+00 | 5.68E−177 | 1.00E−169 |
| Ncr1 | 8 | 22 | 0.951 | 0.086 | 1.60E+00 | 5.24E−178 | 9.28E−171 |
| Klrd1 | 8 | 22 | 0.908 | 0.043 | 1.37E+00 | 8.53E−175 | 1.51E−167 |
| Nkg7 | 8 | 24 | 0.983 | 0.076 | 2.23E+00 | 2.96E−230 | 5.24E−223 |
| Ms4a4b | 8 | 24 | 0.932 | 0.031 | 1.69E+00 | 4.56E−188 | 8.07E−181 |
| Trbc1 | 8 | 24 | 0.852 | 0.076 | 1.34E+00 | 6.70E−188 | 1.19E−180 |
| Ncr1 | 8 | 24 | 0.951 | 0.036 | 1.67E+00 | 3.31E−182 | 5.86E−175 |
| Gimap4 | 8 | 24 | 0.861 | 0.058 | 1.23E+00 | 4.15E−179 | 7.34E−172 |
| Klrd1 | 8 | 24 | 0.908 | 0.076 | 1.32E+00 | 1.93E−169 | 3.41E−162 |
| Nkg7 | 8 | 25 | 0.983 | 0.063 | 2.28E+00 | 1.49E−252 | 2.64E−245 |
| Ncr1 | 8 | 25 | 0.951 | 0.036 | 1.69E+00 | 1.45E−208 | 2.57E−201 |
| Ms4a4b | 8 | 25 | 0.932 | 0.018 | 1.70E+00 | 6.98E−198 | 1.24E−190 |
| Klrd1 | 8 | 25 | 0.908 | 0.014 | 1.43E+00 | 7.14E−184 | 1.26E−176 |
| Fcer1g | 8 | 25 | 0.921 | 0.086 | 1.54E+00 | 1.71E−170 | 3.02E−163 |
| Selplg | 8 | 25 | 0.918 | 0.09 | 1.35E+00 | 2.29E−165 | 4.06E−158 |
| Coro1a | 8 | 25 | 0.91 | 0.1 | 1.36E+00 | 1.65E−164 | 2.93E−157 |
| Ptprcap | 8 | 25 | 0.894 | 0.063 | 1.25E+00 | 1.64E−162 | 2.90E−155 |
| Ms4a4b | 8 | 26 | 0.932 | 0.06 | 1.64E+00 | 5.99E−191 | 1.06E−183 |
| Klrd1 | 8 | 26 | 0.908 | 0.037 | 1.40E+00 | 8.99E−178 | 1.59E−170 |
| Trbc1 | 8 | 26 | 0.852 | 0.069 | 1.32E+00 | 1.40E−176 | 2.48E−169 |
| Gimap4 | 8 | 26 | 0.861 | 0.078 | 1.22E+00 | 3.37E−163 | 5.97E−156 |
| Ccl5 | 8 | 29 | 0.993 | 0.095 | 3.39E+00 | 1.57E−163 | 2.79E−156 |
| Nkg7 | 8 | 30 | 0.983 | 0.044 | 2.29E+00 | 3.81E−172 | 6.74E−165 |
| Ccl5 | 8 | 32 | 0.993 | 0.085 | 3.98E+00 | 1.18E−179 | 2.09E−172 |
| Cd52 | 8 | 32 | 0.989 | 0.094 | 2.16E+00 | 1.45E−164 | 2.56E−157 |
| Cd79b | 9 | 11 | 0.998 | 0.048 | 2.66E+00 | 3.93E−245 | 6.95E−238 |
| Cd79a | 9 | 11 | 0.997 | 0.019 | 2.87E+00 | 5.18E−227 | 9.17E−220 |
| Aicda | 9 | 11 | 0.88 | 0.002 | 1.53E+00 | 6.56E−218 | 1.16E−210 |
| Ms4a1 | 9 | 11 | 0.995 | 0.007 | 2.69E+00 | 5.05E−211 | 8.94E−204 |
| Ebf1 | 9 | 11 | 0.987 | 0.004 | 2.37E+00 | 4.99E−196 | 8.83E−189 |
| Mzb1 | 9 | 11 | 0.985 | 0.029 | 2.48E+00 | 1.05E−174 | 1.85E−167 |
| Rgsl3 | 9 | 11 | 0.948 | 0.002 | 2.04E+00 | 8.72E−171 | 1.54E−163 |
| Hlfx | 9 | 11 | 0.675 | 0.002 | 1.35E+00 | 1.81E−164 | 3.21E−157 |
| Siglecg | 9 | 11 | 0.79 | 0.01 | 1.09E+00 | 6.20E−163 | 1.10E−155 |
| H2-DMb2 | 9 | 11 | 0.98 | 0.036 | 2.23E+00 | 1.30E−160 | 2.31E−153 |
| Smagp | 9 | 11 | 0.866 | 0.007 | 1.37E+00 | 2.76E−159 | 4.88E−152 |
| Cd79a | 9 | 12 | 0.997 | 0.036 | 2.85E+00 | 1.89E−254 | 3.35E−247 |
| Ms4a1 | 9 | 12 | 0.995 | 0.02 | 2.68E+00 | 3.27E−226 | 5.79E−219 |
| Aicda | 9 | 12 | 0.88 | 0.006 | 1.53E+00 | 2.31E−213 | 4.10E−206 |
| Ebf1 | 9 | 12 | 0.987 | 0.009 | 2.36E+00 | 1.32E−206 | 2.34E−199 |
| Mzb1 | 9 | 12 | 0.985 | 0.034 | 2.48E+00 | 3.75E−189 | 6.64E−182 |
| Rgs13 | 9 | 12 | 0.948 | 0.003 | 2.04E+00 | 8.16E−178 | 1.44E−170 |
| H2-DMb2 | 9 | 12 | 0.98 | 0.043 | 2.22E+00 | 1.50E−169 | 2.66E−162 |
| Smagp | 9 | 12 | 0.866 | 0.002 | 1.38E+00 | 6.56E−168 | 1.16E−160 |
| Stmn1 | 9 | 12 | 0.977 | 0.036 | 2.66E+00 | 2.00E−165 | 3.54E−158 |
| Vpreb3 | 9 | 12 | 0.894 | 0.003 | 2.00E+00 | 1.88E−163 | 3.32E−156 |
| Scimp | 9 | 12 | 0.933 | 0.008 | 1.56E+00 | 1.46E−159 | 2.58E−152 |
| Siglecg | 9 | 12 | 0.79 | 0.012 | 1.09E+00 | 4.77E−159 | 8.45E−152 |
| Cd79a | 9 | 14 | 0.997 | 0.081 | 2.74E+00 | 0.00E+00 | 0.00E+00 |
| Ms4a1 | 9 | 14 | 0.995 | 0.053 | 2.62E+00 | 0.00E+00 | 0.00E+00 |
| Mzb1 | 9 | 14 | 0.985 | 0.094 | 2.33E+00 | 0.00E+00 | 0.00E+00 |
| Ebf1 | 9 | 14 | 0.987 | 0.034 | 2.30E+00 | 0.00E+00 | 0.00E+00 |
| Rgs13 | 9 | 14 | 0.948 | 0.014 | 2.02E+00 | 0.00E+00 | 0.00E+00 |
| Pou2af1 | 9 | 14 | 0.963 | 0.023 | 2.01E+00 | 0.00E+00 | 0.00E+00 |
| Vpreb3 | 9 | 14 | 0.894 | 0.014 | 1.98E+00 | 0.00E+00 | 0.00E+00 |
| Chchd10 | 9 | 14 | 0.916 | 0.085 | 1.81E+00 | 0.00E+00 | 0.00E+00 |
| Iglc3 | 9 | 14 | 0.948 | 0.092 | 1.76E+00 | 0.00E+00 | 0.00E+00 |
| Eaf2 | 9 | 14 | 0.9 | 0.034 | 1.56E+00 | 0.00E+00 | 0.00E+00 |
| Aicda | 9 | 14 | 0.88 | 0.008 | 1.52E+00 | 0.00E+00 | 0.00E+00 |
| Apitd1 | 9 | 14 | 0.894 | 0.099 | 1.35E+00 | 3.0E−315 | 5.33E−308 |
| Gcsam | 9 | 14 | 0.874 | 0.056 | 1.48E+00 | 6.17E−300 | 1.09E−292 |
| Smagp | 9 | 14 | 0.866 | 0.085 | 1.29E+00 | 8.32E−283 | 1.47E−275 |
| Cdl9 | 9 | 14 | 0.801 | 0.014 | 1.09E+00 | 2.81E−278 | 4.98E−271 |
| Iglc2 | 9 | 14 | 0.847 | 0.063 | 1.66E+00 | 2.23E−276 | 3.94E−269 |
| Blnk | 9 | 14 | 0.848 | 0.088 | 1.22E+00 | 2.20E−259 | 3.90E−252 |
| Bfsp2 | 9 | 14 | 0.775 | 0.033 | 1.36E+00 | 1.88E−247 | 3.34E−240 |
| Neil1 | 9 | 14 | 0.792 | 0.058 | 1.20E+00 | 4.58E−239 | 8.10E−232 |
| Cd81 | 9 | 14 | 0.838 | 0.094 | 1.12E+00 | 6.41E−239 | 1.14E−231 |
| Csrp2 | 9 | 14 | 0.742 | 0.027 | 1.00E+00 | 5.87E−225 | 1.04E−217 |
| Fcmr | 9 | 14 | 0.731 | 0.023 | 9.44E−01 | 3.68E−222 | 6.52E−215 |
| Hlfx | 9 | 14 | 0.675 | 0.006 | 1.34E+00 | 1.16E−219 | 2.05E−212 |
| Apobec1 | 9 | 14 | 0.788 | 0.081 | 1.00E+00 | 1.33E−215 | 2.35E−208 |
| Id3 | 9 | 14 | 0.722 | 0.031 | 1.16E+00 | 2.68E−207 | 4.75E−200 |
| Slpr2 | 9 | 14 | 0.705 | 0.02 | 9.16E−01 | 9.00E−207 | 1.59E−199 |
| Lipc | 9 | 14 | 0.66 | 0.003 | 8.78E−01 | 1.64E−203 | 2.90E−196 |
| Myl4 | 9 | 14 | 0.714 | 0.044 | 1.18E+00 | 5.61E−199 | 9.93E−192 |
| Asf1b | 9 | 14 | 0.75 | 0.075 | 1.16E+00 | 3.18E−196 | 5.63E−189 |
| Sh2b2 | 9 | 14 | 0.724 | 0.049 | 9.44E−01 | 1.11E−195 | 1.97E−188 |
| Nuggc | 9 | 14 | 0.586 | 0.002 | 7.58E−01 | 1.75E−170 | 3.10E−163 |
| Cd22 | 9 | 14 | 0.642 | 0.023 | 8.07E−01 | 8.94E−170 | 1.58E−162 |
| Cd79a | 9 | 17 | 0.997 | 0.082 | 2.78E+00 | 9.84E−183 | 1.74E−175 |
| Ms4a1 | 9 | 17 | 0.995 | 0.035 | 2.66E+00 | 5.67E−160 | l.OOE−152 |
| Stmn1 | 9 | 20 | 0.977 | 0.081 | 2.52E+00 | 2.71E−162 | 4.80E−155 |
| Cd79a | 9 | 21 | 0.997 | 0.098 | 2.76E+00 | 0.00E+00 | 0.00E+00 |
| Ms4a1 | 9 | 21 | 0.995 | 0.034 | 2.66E+00 | 4.15E−303 | 7.35E−296 |
| Ebf1 | 9 | 21 | 0.987 | 0.021 | 2.35E+00 | 5.78E−277 | 1.02E−269 |
| Pou2af1 | 9 | 21 | 0.963 | 0.038 | 1.99E+00 | 9.91E−246 | 1.75E−238 |
| Rgs13 | 9 | 21 | 0.948 | 0.021 | 2.02E+00 | 1.90E−221 | 3.36E−214 |
| Iglc3 | 9 | 21 | 0.948 | 0.081 | 1.84E+00 | 2.35E−204 | 4.16E−197 |
| Tnfrsf13c | 9 | 21 | 0.906 | 0.043 | 1.62E+00 | 5.72E−184 | 1.01E−176 |
| Eaf2 | 9 | 21 | 0.9 | 0.026 | 1.58E+00 | 8.29E−184 | 1.47E−176 |
| Aicda | 9 | 21 | 0.88 | 0.004 | 1.53E+00 | 2.66E−176 | 4.72E−169 |
| Vpreb3 | 9 | 21 | 0.894 | 0.034 | 1.96E+00 | 1.48E−174 | 2.61E−167 |
| Cd79a | 9 | 22 | 0.997 | 0.06 | 2.75E+00 | 5.18E−216 | 9.16E−209 |
| Ms4a1 | 9 | 22 | 0.995 | 0.039 | 2.64E+00 | 6.47E−211 | 1.14E−203 |
| Ebf1 | 9 | 22 | 0.987 | 0.043 | 2.29E+00 | 1.98E−188 | 3.51E−181 |
| Mzb1 | 9 | 22 | 0.985 | 0.065 | 2.45E+00 | 2.95E−179 | 5.21E−172 |
| Stmn1 | 9 | 22 | 0.977 | 0.034 | 2.67E+00 | 8.71E−174 | 1.54E−166 |
| Cd24a | 9 | 22 | 0.976 | 0.047 | 2.30E+00 | 1.89E−161 | 3.35E−154 |
| Pou2af1 | 9 | 22 | 0.963 | 0.013 | 2.04E+00 | 2.98E−158 | 5.27E−151 |
| Cd79a | 9 | 24 | 0.997 | 0.049 | 2.84E+00 | 8.48E−229 | 1.50E−221 |
| Ms4a1 | 9 | 24 | 0.995 | 0.045 | 2.65E+00 | 2.20E−211 | 3.89E−204 |
| Mzb1 | 9 | 24 | 0.985 | 0.058 | 2.46E+00 | 1.11E−181 | 1.97E−174 |
| Stmn1 | 9 | 24 | 0.977 | 0.058 | 2.59E+00 | 6.50E−162 | 1.15E−154 |
| Pou2af1 | 9 | 24 | 0.963 | 0.009 | 2.04E+00 | 2.88E−159 | 5.10E−152 |
| Cd79b | 9 | 26 | 0.998 | 0.092 | 2.60E+00 | 4.40E−200 | 7.79E−193 |
| Cd79a | 9 | 26 | 0.997 | 0.018 | 2.86E+00 | 3.10E−193 | 5.48E−186 |
| Ms4a1 | 9 | 26 | 0.995 | 0.014 | 2.68E+00 | 3.48E−188 | 6.15E−181 |
| Ebf1 | 9 | 26 | 0.987 | 0.005 | 2.37E+00 | 1.71E−174 | 3.02E−167 |
| Mzb1 | 9 | 26 | 0.985 | 0.041 | 2.46E+00 | 5.42E−159 | 9.58E−152 |
| Cd79a | 9 | 34 | 0.997 | 0.05 | 2.82E+00 | 5.35E−169 | 9.48E−162 |
| Cd79b | 9 | 35 | 0.998 | 0.081 | 2.61E+00 | 5.26E−166 | 9.31E−159 |
| Cd79a | 10 | 11 | 0.998 | 0.019 | 2.63E+00 | 0.00E+00 | 0.00E+00 |
| Cd79b | 10 | 11 | 0.995 | 0.048 | 2.50E+00 | 0.00E+00 | 0.00E+00 |
| Ms4a1 | 10 | 11 | 0.991 | 0.007 | 2.39E+00 | 0.00E+00 | 0.00E+00 |
| Ighg1 | 10 | 11 | 0.844 | 0.072 | 2.28E+00 | 0.00E+00 | 0.00E+00 |
| Ebf1 | 10 | 11 | 0.992 | 0.004 | 2.14E+00 | 0.00E+00 | 0.00E+00 |
| Mzb1 | 10 | 11 | 0.97 | 0.029 | 2.05E+00 | 0.00E+00 | 0.00E+00 |
| H2-DMb2 | 10 | 11 | 0.971 | 0.036 | 2.04E+00 | 0.00E+00 | 0.00E+00 |
| Ifi30 | 10 | 11 | 0.922 | 0.064 | 1.75E+00 | 0.00E+00 | 0.00E+00 |
| Rgs13 | 10 | 11 | 0.907 | 0.002 | 1.62E+00 | 0.00E+00 | 0.00E+00 |
| Iglc3 | 10 | 11 | 0.89 | 0.024 | 1.50E+00 | 0.00E+00 | 0.00E+00 |
| Mef2c | 10 | 11 | 0.922 | 0.048 | 1.45E+00 | 0.00E+00 | 0.00E+00 |
| Tnfrsf13c | 10 | 11 | 0.864 | 0.016 | 1.44E+00 | 0.00E+00 | 0.00E+00 |
| Sypl | 10 | 11 | 0.903 | 0.037 | 1.42E+00 | 0.00E+00 | 0.00E+00 |
| Cd24a | 10 | 11 | 0.842 | 0.015 | 1.33E+00 | 0.00E+00 | 0.00E+00 |
| Aicda | 10 | 11 | 0.833 | 0.002 | 1.33E+00 | 0.00E+00 | 0.00E+00 |
| Pou2af1 | 10 | 11 | 0.84 | 0.007 | 1.29E+00 | 0.00E+00 | 0.00E+00 |
| Scimp | 10 | 11 | 0.855 | 0.017 | 1.26E+00 | 0.00E+00 | 0.00E+00 |
| Siglecg | 10 | 11 | 0.794 | 0.01 | 1.04E+00 | 3.4E−316 | 0.00E+00 |
| Basp1 | 10 | 11 | 0.821 | 0.026 | 1.24E+00 | 1.0E−310 | 1.88E−303 |
| Iglc2 | 10 | 11 | 0.825 | 0.027 | 1.58E+00 | 6.08E−307 | 1.08E−299 |
| Eaf2 | 10 | 11 | 0.798 | 0.006 | 1.21E+00 | 1.05E−301 | 1.86E−294 |
| Apoe | 10 | 11 | 0.81 | 0.037 | 1.86E+00 | 6.28E−297 | 1.11E−289 |
| Iglc1 | 10 | 11 | 0.827 | 0.05 | 2.19E+00 | 1.73E−277 | 3.06E−270 |
| H2-Ob | 10 | 11 | 0.741 | 0.007 | 9.24E−01 | 3.68E−269 | 6.53E−262 |
| Cd19 | 10 | 11 | 0.745 | 0.004 | 9.18E−01 | 1.38E−266 | 2.45E−259 |
| Vpreb3 | 10 | 11 | 0.743 | 0.012 | 1.31E+00 | 5.68E−263 | 1.01E−255 |
| Plekho1 | 10 | 11 | 0.731 | 0.008 | 9.07E−01 | 2.71E−261 | 4.80E−254 |
| Fcmr | 10 | 11 | 0.732 | 0.004 | 9.61E−01 | 9.71E−259 | 1.72E−251 |
| Ctsh | 10 | 11 | 0.816 | 0.07 | 1.03E+00 | 6.46E−243 | 1.14E−235 |
| Pkig | 10 | 11 | 0.788 | 0.055 | 1.09E+00 | 7.22E−230 | 1.28E−222 |
| Smagp | 10 | 11 | 0.691 | 0.007 | 9.10E−01 | 1.52E−229 | 2.68E−222 |
| Gcsam | 10 | 11 | 0.674 | 0.008 | 9.54E−01 | 8.41E−220 | 1.49E−212 |
| Cd22 | 10 | 11 | 0.64 | 0.002 | 7.95E−01 | 2.04E−212 | 3.61E−205 |
| Pou2f2 | 10 | 11 | 0.792 | 0.092 | 9.93E−01 | 1.12E−211 | 1.98E−204 |
| Spib | 10 | 11 | 0.706 | 0.022 | 9.31E−01 | 1.39E−209 | 2.45E−202 |
| Neil1 | 10 | 11 | 0.705 | 0.037 | 9.03E−01 | 2.01E−201 | 3.56E−194 |
| Apobec1 | 10 | 11 | 0.68 | 0.018 | 7.94E−01 | 9.30E−201 | 1.65E−193 |
| Cd81 | 10 | 11 | 0.722 | 0.042 | 8.90E−01 | 9.65E−196 | 1.71E−188 |
| Blnk | 10 | 11 | 0.676 | 0.024 | 8.51E−01 | 5.58E−191 | 9.88E−184 |
| Sh2b2 | 10 | 11 | 0.575 | 0.004 | 6.58E−01 | 8.74E−185 | 1.55E−177 |
| Dok3 | 10 | 11 | 0.602 | 0.004 | 7.14E−01 | 2.91E−181 | 5.15E−174 |
| Slpr2 | 10 | 11 | 0.602 | 0.01 | 7.65E−01 | 1.97E−176 | 3.49E−169 |
| Bfsp2 | 10 | 11 | 0.649 | 0.03 | 9.62E−01 | 1.58E−171 | 2.80E−164 |
| Apitd1 | 10 | 11 | 0.641 | 0.023 | 7.57E−01 | 3.86E−169 | 6.83E−162 |
| H2-Oa | 10 | 11 | 0.647 | 0.039 | 7.65E−01 | 2.66E−166 | 4.71E−159 |
| Cd79a | 10 | 12 | 0.998 | 0.036 | 2.61E+00 | 0.00E+00 | 0.00E+00 |
| Ms4a1 | 10 | 12 | 0.991 | 0.02 | 2.38E+00 | 0.00E+00 | 0.00E+00 |
| Ighg1 | 10 | 12 | 0.844 | 0.073 | 2.27E+00 | 0.00E+00 | 0.00E+00 |
| Ebf1 | 10 | 12 | 0.992 | 0.009 | 2.13E+00 | 0.00E+00 | 0.00E+00 |
| Mzb1 | 10 | 12 | 0.97 | 0.034 | 2.04E+00 | 0.00E+00 | 0.00E+00 |
| H2-DMb2 | 10 | 12 | 0.971 | 0.043 | 2.02E+00 | 0.00E+00 | 0.00E+00 |
| Ifi30 | 10 | 12 | 0.922 | 0.069 | 1.73E+00 | 0.00E+00 | 0.00E+00 |
| Rgs13 | 10 | 12 | 0.907 | 0.003 | 1.62E+00 | 0.00E+00 | 0.00E+00 |
| Iglc3 | 10 | 12 | 0.89 | 0.019 | 1.50E+00 | 0.00E+00 | 0.00E+00 |
| Mef2c | 10 | 12 | 0.922 | 0.021 | 1.47E+00 | 0.00E+00 | 0.00E+00 |
| Tnfrsf13c | 10 | 12 | 0.864 | 0.009 | 1.45E+00 | 0.00E+00 | 0.00E+00 |
| Sypl | 10 | 12 | 0.903 | 0.089 | 1.36E+00 | 0.00E+00 | 0.00E+00 |
| Aicda | 10 | 12 | 0.833 | 0.006 | 1.32E+00 | 0.00E+00 | 0.00E+00 |
| Pou2af1 | 10 | 12 | 0.84 | 0.006 | 1.29E+00 | 0.00E+00 | 0.00E+00 |
| Scimp | 10 | 12 | 0.855 | 0.008 | 1.27E+00 | 0.00E+00 | 0.00E+00 |
| Siglecg | 10 | 12 | 0.794 | 0.012 | 1.04E+00 | 2.28E−307 | 4.03E−300 |
| Eaf2 | 10 | 12 | 0.798 | 0.005 | 1.21E+00 | 5.39E−306 | 9.55E−299 |
| Iglc2 | 10 | 12 | 0.825 | 0.037 | 1.57E+00 | 1.75E−295 | 3.10E−288 |
| Apoe | 10 | 12 | 0.81 | 0.053 | 1.84E+00 | 1.86E−279 | 3.29E−272 |
| Vpreb3 | 10 | 12 | 0.743 | 0.003 | 1.31E+00 | 2.87E−275 | 5.08E−268 |
| Cd24a | 10 | 12 | 0.842 | 0.069 | 1.25E+00 | 7.23E−268 | 1.28E−260 |
| H2-Ob | 10 | 12 | 0.741 | 0.01 | 9.21E−01 | 9.22E−262 | 1.63E−254 |
| Iglc1 | 10 | 12 | 0.827 | 0.07 | 2.18E+00 | 1.01E−261 | 1.79E−254 |
| Plekho1 | 10 | 12 | 0.731 | 0.01 | 9.05E−01 | 1.95E−255 | 3.44E−248 |
| Cd19 | 10 | 12 | 0.745 | 0.012 | 9.10E−01 | 3.19E−253 | 5.65E−246 |
| Fcmr | 10 | 12 | 0.732 | 0.012 | 9.52E−01 | 1.68E−244 | 2.97E−237 |
| Smagp | 10 | 12 | 0.691 | 0.002 | 9.15E−01 | 2.09E−237 | 3.70E−230 |
| Pkig | 10 | 12 | 0.788 | 0.064 | 1.08E+00 | 1.18E−226 | 2.08E−219 |
| Ctsh | 10 | 12 | 0.816 | 0.098 | 1.00E+00 | 2.17E−223 | 3.84E−216 |
| Lat2 | 10 | 12 | 0.763 | 0.047 | 9.49E−01 | 7.05E−223 | 1.25E−215 |
| Gcsam | 10 | 12 | 0.674 | 0.007 | 9.55E−01 | 4.03E−221 | 7.13E−214 |
| Spib | 10 | 12 | 0.706 | 0.021 | 9.31E−01 | 2.91E−212 | 5.15E−205 |
| Apobec1 | 10 | 12 | 0.68 | 0.012 | 8.04E−01 | 1.39E−211 | 2.47E−204 |
| Cd22 | 10 | 12 | 0.64 | 0.007 | 7.91E−01 | 2.53E−202 | 4.48E−195 |
| Neil1 | 10 | 12 | 0.705 | 0.042 | 8.98E−01 | 2.33E−197 | 4.12E−190 |
| Il21r | 10 | 12 | 0.655 | 0.016 | 8.25E−01 | 1.03E−190 | 1.82E−183 |
| Bfsp2 | 10 | 12 | 0.649 | 0.015 | 9.76E−01 | 4.45E−190 | 7.87E−183 |
| Blnk | 10 | 12 | 0.676 | 0.03 | 8.44E−01 | 2.70E−185 | 4.78E−178 |
| Dok3 | 10 | 12 | 0.602 | 0.006 | 7.12E−01 | 1.02E−178 | 1.81E−171 |
| Sh2b2 | 10 | 12 | 0.575 | 0.009 | 6.54E−01 | 2.16E−173 | 3.83E−166 |
| Slpr2 | 10 | 12 | 0.602 | 0.016 | 7.59E−01 | 2.93E−168 | 5.18E−161 |
| Apitd1 | 10 | 12 | 0.641 | 0.033 | 7.47E−01 | 4.53E−161 | 8.02E−154 |
| Napsa | 10 | 12 | 0.687 | 0.068 | 8.57E−01 | 1.52E−158 | 2.69E−151 |
| H2-Aa | 10 | 13 | 0.999 | 0.076 | 3.49E+00 | 3.61E−168 | 6.40E−161 |
| Cd79a | 10 | 14 | 0.998 | 0.081 | 2.50E+00 | 0.00E+00 | 0.00E+00 |
| Ms4a1 | 10 | 14 | 0.991 | 0.053 | 2.31E+00 | 0.00E+00 | 0.00E+00 |
| Ebf1 | 10 | 14 | 0.992 | 0.034 | 2.07E+00 | 0.00E+00 | 0.00E+00 |
| Mzb1 | 10 | 14 | 0.97 | 0.094 | 1.90E+00 | 0.00E+00 | 0.00E+00 |
| Rgs13 | 10 | 14 | 0.907 | 0.014 | 1.60E+00 | 0.00E+00 | 0.00E+00 |
| Iglc3 | 10 | 14 | 0.89 | 0.092 | 1.36E+00 | 0.00E+00 | 0.00E+00 |
| Aicda | 10 | 14 | 0.833 | 0.008 | 1.31E+00 | 0.00E+00 | 0.00E+00 |
| Pou2af1 | 10 | 14 | 0.84 | 0.023 | 1.26E+00 | 0.00E+00 | 0.00E+00 |
| Eaf2 | 10 | 14 | 0.798 | 0.034 | 1.17E+00 | 2.4E−310 | 4.25E−303 |
| Tnfrsf13c | 10 | 14 | 0.864 | 0.091 | 1.35E+00 | 0.00E+00 | 6.31E−302 |
| Iglc2 | 10 | 14 | 0.825 | 0.063 | 1.47E+00 | 3.70E−287 | 6.55E−280 |
| Cd19 | 10 | 14 | 0.745 | 0.014 | 9.08E−01 | 2.99E−273 | 5.30E−266 |
| Vpreb3 | 10 | 14 | 0.743 | 0.014 | 1.30E+00 | 1.62E−259 | 2.88E−252 |
| Fcmr | 10 | 14 | 0.732 | 0.023 | 9.28E−01 | 2.95E−258 | 5.22E−251 |
| Neil1 | 10 | 14 | 0.705 | 0.058 | 8.77E−01 | 8.25E−220 | 1.46E−212 |
| Bfsp2 | 10 | 14 | 0.649 | 0.033 | 9.55E−01 | 2.51E−218 | 4.44E−211 |
| Blnk | 10 | 14 | 0.676 | 0.088 | 7.73E−01 | 1.36E−210 | 2.41E−203 |
| Smagp | 10 | 14 | 0.691 | 0.085 | 8.24E−01 | 2.47E−207 | 4.38E−200 |
| Cd81 | 10 | 14 | 0.722 | 0.094 | 8.11E−01 | 2.30E−205 | 4.07E−198 |
| Cd22 | 10 | 14 | 0.64 | 0.023 | 7.68E−01 | 1.11E−204 | 1.96E−197 |
| Apitd1 | 10 | 14 | 0.641 | 0.099 | 6.56E−01 | 5.55E−200 | 9.83E−193 |
| Slpr2 | 10 | 14 | 0.602 | 0.02 | 7.49E−01 | 8.78E−195 | 1.55E−187 |
| Gcsam | 10 | 14 | 0.674 | 0.056 | 8.38E−01 | 1.22E−194 | 2.15E−187 |
| Apobec1 | 10 | 14 | 0.68 | 0.081 | 7.29E−01 | 2.83E−193 | 5.00E−186 |
| Lipc | 10 | 14 | 0.523 | 0.003 | 6.71E−01 | 3.47E−184 | 6.15E−177 |
| Chchd10 | 10 | 14 | 0.594 | 0.085 | 7.19E−01 | 1.96E−159 | 3.46E−152 |
| Cd79a | 10 | 17 | 0.998 | 0.082 | 2.54E+00 | 0.00E+00 | 0.00E+00 |
| Ms4a1 | 10 | 17 | 0.991 | 0.035 | 2.36E+00 | 0.00E+00 | 0.00E+00 |
| Ebf1 | 10 | 17 | 0.992 | 0.018 | 2.11E+00 | 0.00E+00 | 0.00E+00 |
| H2-DMb2 | 10 | 17 | 0.971 | 0.059 | 2.00E+00 | 2.87E−294 | 5.09E−287 |
| Mzb1 | 10 | 17 | 0.97 | 0.041 | 2.04E+00 | 9.13E−292 | 1.62E−284 |
| Rgs13 | 10 | 17 | 0.907 | 0.006 | 1.62E+00 | 8.52E−237 | 1.51E−229 |
| Mef2c | 10 | 17 | 0.922 | 0.041 | 1.44E+00 | 5.71E−228 | 1.01E−220 |
| Iglc3 | 10 | 17 | 0.89 | 0.035 | 1.48E+00 | 4.72E−203 | 8.36E−196 |
| Scimp | 10 | 17 | 0.855 | 0.018 | 1.26E+00 | 9.09E−189 | 1.61E−181 |
| Aicda | 10 | 17 | 0.833 | 0.006 | 1.32E+00 | 1.73E−186 | 3.07E−179 |
| Tnfrsf13c | 10 | 17 | 0.864 | 0.038 | 1.42E+00 | l.OOE−183 | 1.78E−176 |
| Ighg1 | 10 | 17 | 0.844 | 0.082 | 2.27E+00 | 1.20E−177 | 2.12E−170 |
| Pou2af1 | 10 | 17 | 0.84 | 0.023 | 1.27E+00 | 5.60E−177 | 9.91E−170 |
| Cd24a | 10 | 17 | 0.842 | 0.035 | 1.31E+00 | 1.10E−170 | 1.94E−163 |
| Siglecg | 10 | 17 | 0.794 | 0.009 | 1.04E+00 | 9.13E−166 | 1.62E−158 |
| Eaf2 | 10 | 17 | 0.798 | 0.009 | 1.20E+00 | 9.41E−163 | 1.66E−155 |
| Ebf1 | 10 | 18 | 0.992 | 0.036 | 2.11E+00 | 1.11E−295 | 1.97E−288 |
| Ms4a1 | 10 | 18 | 0.991 | 0.078 | 2.32E+00 | 8.46E−294 | 1.50E−286 |
| H2-DMb2 | 10 | 18 | 0.971 | 0.096 | 1.95E+00 | 1.38E−246 | 2.44E−239 |
| Mzb1 | 10 | 18 | 0.97 | 0.053 | 2.03E+00 | 4.86E−245 | 8.61E−238 |
| Mef2c | 10 | 18 | 0.922 | 0.021 | 1.48E+00 | 7.94E−201 | 1.41E−193 |
| Rgs13 | 10 | 18 | 0.907 | 0.018 | 1.60E+00 | 5.24E−190 | 9.27E−183 |
| Ifi30 | 10 | 18 | 0.922 | 0.078 | 1.73E+00 | 2.12E−178 | 3.75E−171 |
| Iglc3 | 10 | 18 | 0.89 | 0.053 | 1.47E+00 | 1.78E−161 | 3.15E−154 |
| Cd79a | 10 | 19 | 0.998 | 0.072 | 2.58E+00 | 7.16E−230 | 1.27E−222 |
| Cd79b | 10 | 19 | 0.995 | 0.064 | 2.49E+00 | 6.93E−205 | 1.23E−197 |
| Ebf1 | 10 | 19 | 0.992 | 0.016 | 2.13E+00 | 4.79E−199 | 8.48E−192 |
| Ms4a1 | 10 | 19 | 0.991 | 0.028 | 2.37E+00 | 1.22E−192 | 2.17E−185 |
| Rgs13 | 10 | 20 | 0.907 | 0.064 | 1.48E+00 | 2.65E−185 | 4.70E−178 |
| Aicda | 10 | 20 | 0.833 | 0.017 | 1.31E+00 | 4.12E−180 | 7.29E−173 |
| Scimp | 10 | 20 | 0.855 | 0.059 | 1.22E+00 | 2.59E−161 | 4.59E−154 |
| Cd79a | 10 | 21 | 0.998 | 0.098 | 2.53E+00 | 5.07E−300 | 8.97E−293 |
| Ms4a1 | 10 | 21 | 0.991 | 0.034 | 2.36E+00 | 1.40E−227 | 2.48E−220 |
| Aicda | 10 | 21 | 0.833 | 0.004 | 1.32E+00 | 1.38E−198 | 2.45E−191 |
| Ebf1 | 10 | 21 | 0.992 | 0.021 | 2.12E+00 | 1.28E−193 | 2.27E−186 |
| Rgs13 | 10 | 21 | 0.907 | 0.021 | 1.60E+00 | 3.72E−188 | 6.58E−181 |
| Iglc3 | 10 | 21 | 0.89 | 0.081 | 1.43E+00 | 7.41E−186 | 1.31E−178 |
| Pou2af1 | 10 | 21 | 0.84 | 0.038 | 1.24E+00 | 5.44E−183 | 9.63E−176 |
| Eaf2 | 10 | 21 | 0.798 | 0.026 | 1.19E+00 | 1.06E−168 | 1.88E−161 |
| Tnfrsf13c | 10 | 21 | 0.864 | 0.043 | 1.41E+00 | 8.31E−166 | 1.47E−158 |
| Cd79a | 10 | 22 | 0.998 | 0.06 | 2.51E+00 | 5.54E−304 | 9.80E−297 |
| Ms4a1 | 10 | 22 | 0.991 | 0.039 | 2.34E+00 | 8.01E−280 | 1.42E−272 |
| Ebf1 | 10 | 22 | 0.992 | 0.043 | 2.06E+00 | 2.12E−267 | 3.75E−260 |
| Mzb1 | 10 | 22 | 0.97 | 0.065 | 2.01E+00 | 3.91E−244 | 6.92E−237 |
| Rgs13 | 10 | 22 | 0.907 | 0.013 | 1.61E+00 | 5.35E−209 | 9.47E−202 |
| Iglc3 | 10 | 22 | 0.89 | 0.069 | 1.43E+00 | 3.11E−182 | 5.51E−175 |
| Aicda | 10 | 22 | 0.833 | 0.017 | 1.31E+00 | 2.75E−180 | 4.88E−173 |
| Pou2af1 | 10 | 22 | 0.84 | 0.013 | 1.28E+00 | 1.97E−176 | 3.50E−169 |
| Tnfrsf13c | 10 | 22 | 0.864 | 0.017 | 1.43E+00 | 1.11E−175 | 1.97E−168 |
| Cd24a | 10 | 22 | 0.842 | 0.047 | 1.30E+00 | 6.21E−160 | 1.10E−152 |
| Cd79a | 10 | 24 | 0.998 | 0.049 | 2.60E+00 | 0.00E+00 | 0.00E+00 |
| Ms4a1 | 10 | 24 | 0.991 | 0.045 | 2.35E+00 | 5.18E−283 | 9.17E−276 |
| Mzb1 | 10 | 24 | 0.97 | 0.058 | 2.02E+00 | 1.75E−255 | 3.10E−248 |
| Rgs13 | 10 | 24 | 0.907 | 0.013 | 1.60E+00 | 3.34E−202 | 5.91E−195 |
| Aicda | 10 | 24 | 0.833 | 0.013 | 1.30E+00 | 5.11E−175 | 9.05E−168 |
| Pou2af1 | 10 | 24 | 0.84 | 0.009 | 1.29E+00 | 2.44E−174 | 4.31E−167 |
| Iglc3 | 10 | 24 | 0.89 | 0.081 | 1.39E+00 | 7.47E−174 | 1.32E−166 |
| Tnfrsf13c | 10 | 24 | 0.864 | 0.013 | 1.44E+00 | 2.27E−173 | 4.01E−166 |
| Scimp | 10 | 24 | 0.855 | 0.031 | 1.25E+00 | 4.54E−170 | 8.04E−163 |
| Eaf2 | 10 | 24 | 0.798 | 0 | 1.21E+00 | 2.57E−161 | 4.54E−154 |
| Cd79a | 10 | 25 | 0.998 | 0.027 | 2.62E+00 | 3.18E−255 | 5.64E−248 |
| Cd79b | 10 | 25 | 0.995 | 0.009 | 2.54E+00 | 3.64E−242 | 6.45E−235 |
| Ms4a1 | 10 | 25 | 0.991 | 0 | 2.40E+00 | 3.99E−234 | 7.06E−227 |
| Coro1a | 10 | 25 | 0.989 | 0.1 | 2.32E+00 | 2.55E−223 | 4.51E−216 |
| Mzb1 | 10 | 25 | 0.97 | 0.023 | 2.05E+00 | 3.06E−190 | 5.42E−183 |
| Cd37 | 10 | 25 | 0.965 | 0.014 | 1.79E+00 | 4.75E−190 | 8.40E−183 |
| H2-DMb2 | 10 | 25 | 0.971 | 0.045 | 2.03E+00 | 5.72E−189 | 1.01E−181 |
| Rhoh | 10 | 25 | 0.96 | 0.036 | 1.69E+00 | 2.97E−177 | 5.26E−170 |
| Laptm5 | 10 | 25 | 0.96 | 0.1 | 1.69E+00 | 4.12E−159 | 7.28E−152 |
| Limd2 | 10 | 25 | 0.962 | 0.1 | 1.72E+00 | 1.90E−158 | 3.36E−151 |
| Cd79a | 10 | 26 | 0.998 | 0.018 | 2.62E+00 | 8.82E−302 | 1.56E−294 |
| Cd79b | 10 | 26 | 0.995 | 0.092 | 2.44E+00 | 1.67E−298 | 2.95E−291 |
| Ms4a1 | 10 | 26 | 0.991 | 0.014 | 2.38E+00 | 7.48E−275 | 1.32E−267 |
| Ebf1 | 10 | 26 | 0.992 | 0.005 | 2.14E+00 | 5.80E−273 | 1.03E−265 |
| H2-DMb2 | 10 | 26 | 0.971 | 0.069 | 1.98E+00 | 1.09E−246 | 1.94E−239 |
| Mzb1 | 10 | 26 | 0.97 | 0.041 | 2.03E+00 | 1.91E−245 | 3.38E−238 |
| Mef2c | 10 | 26 | 0.922 | 0.037 | 1.44E+00 | 8.93E−198 | 1.58E−190 |
| Ifi30 | 10 | 26 | 0.922 | 0.092 | 1.69E+00 | 1.66E−187 | 2.94E−180 |
| Iglc3 | 10 | 26 | 0.89 | 0.069 | 1.41E+00 | 8.92E−169 | 1.58E−161 |
| Aicda | 10 | 26 | 0.833 | 0.005 | 1.32E+00 | 3.12E−167 | 5.52E−160 |
| Tnfrsf13c | 10 | 26 | 0.864 | 0.009 | 1.44E+00 | 7.36E−167 | 1.30E−159 |
| Pou2af1 | 10 | 26 | 0.84 | 0.009 | 1.29E+00 | 2.00E−162 | 3.53E−155 |
| Scimp | 10 | 26 | 0.855 | 0.018 | 1.26E+00 | 1.38E−161 | 2.44E−154 |
| Cd79a | 10 | 30 | 0.998 | 0.052 | 2.57E+00 | 3.89E−191 | 6.88E−184 |
| Cd79b | 10 | 30 | 0.995 | 0.059 | 2.45E+00 | 3.12E−172 | 5.52E−165 |
| Ms4a1 | 10 | 30 | 0.991 | 0.022 | 2.35E+00 | 1.41E−162 | 2.50E−155 |
| Ebf1 | 10 | 30 | 0.992 | 0.022 | 2.12E+00 | 8.97E−162 | 1.59E−154 |
| Cd79a | 10 | 32 | 0.998 | 0.009 | 2.64E+00 | 2.54E−167 | 4.50E−160 |
| Cd79a | 10 | 33 | 0.998 | 0.057 | 2.59E+00 | 3.57E−182 | 6.31E−175 |
| Cd79b | 10 | 33 | 0.995 | 0.076 | 2.48E+00 | 1.51E−174 | 2.67E−167 |
| Ms4a1 | 10 | 33 | 0.991 | 0.01 | 2.39E+00 | 8.34E−160 | 1.48E−152 |
| Cd79b | 10 | 35 | 0.995 | 0.081 | 2.46E+00 | 1.84E−177 | 3.26E−170 |
| H2-DMb2 | 10 | 35 | 0.971 | 0.101 | 1.94E+00 | 1.09E−160 | 1.93E−153 |
| Xcl1 | 11 | 12 | 0.908 | 0.033 | 1.93E+00 | 0.00E+00 | 0.00E+00 |
| Ncr1 | 11 | 12 | 0.952 | 0.076 | 1.65E+00 | 0.00E+00 | 0.00E+00 |
| Klrd1 | 11 | 12 | 0.853 | 0.02 | 1.28E+00 | 0.00E+00 | 0.00E+00 |
| Klrk1 | 11 | 12 | 0.88 | 0.059 | 1.25E+00 | 0.00E+00 | 0.00E+00 |
| Ms4a4b | 11 | 12 | 0.817 | 0.051 | 1.21E+00 | 4.74E−300 | 8.39E−293 |
| Car2 | 11 | 12 | 0.783 | 0.1 | 1.23E+00 | 7.03E−250 | 1.24E−242 |
| Klrc1 | 11 | 12 | 0.595 | 0.017 | 1.02E+00 | 2.45E−194 | 4.34E−187 |
| Klrblf | 11 | 12 | 0.579 | 0.025 | 7.00E−01 | 1.51E−182 | 2.67E−175 |
| Fasl | 11 | 12 | 0.598 | 0.035 | 6.67E−01 | 3.16E−176 | 5.60E−169 |
| Ncr1 | 11 | 14 | 0.952 | 0.077 | 1.64E+00 | 1.55E−194 | 2.74E−187 |
| Ccl5 | 11 | 18 | 0.973 | 0.085 | 3.04E+00 | 7.30E−233 | 1.29E−225 |
| Ncr1 | 11 | 18 | 0.952 | 0.007 | 1.75E+00 | 1.45E−231 | 2.57E−224 |
| Fcer1g | 11 | 18 | 0.966 | 0.05 | 1.65E+00 | 4.66E−229 | 8.25E−222 |
| Tyrobp | 11 | 18 | 0.945 | 0.06 | 1.98E+00 | 3.97E−219 | 7.03E−212 |
| Cd7 | 11 | 18 | 0.924 | 0.05 | 1.91E+00 | 5.81E−203 | 1.03E−195 |
| Xcl1 | 11 | 18 | 0.908 | 0.039 | 1.63E+00 | 2.51E−179 | 4.45E−172 |
| Klrk1 | 11 | 18 | 0.88 | 0.021 | 1.30E+00 | 6.65E−177 | 1.18E−169 |
| Klrd1 | 11 | 18 | 0.853 | 0.028 | 1.26E+00 | 6.33E−159 | 1.12E−151 |
| Ncr1 | 11 | 20 | 0.952 | 0.089 | 1.64E+00 | 2.56E−176 | 4.53E−169 |
| Nkg7 | 11 | 24 | 0.964 | 0.076 | 1.79E+00 | 6.88E−184 | 1.22E−176 |
| Ncr1 | 11 | 24 | 0.952 | 0.036 | 1.70E+00 | 1.22E−160 | 2.16E−153 |
| Gimap4 | 11 | 24 | 0.944 | 0.058 | 1.50E+00 | 5.77E−159 | 1.02E−151 |
| Nkg7 | 11 | 25 | 0.964 | 0.063 | 1.84E+00 | 2.29E−193 | 4.06E−186 |
| Ncr1 | 11 | 25 | 0.952 | 0.036 | 1.72E+00 | 8.48E−185 | 1.50E−177 |
| Fcer1g | 11 | 25 | 0.966 | 0.086 | 1.57E+00 | 4.05E−177 | 7.18E−170 |
| Xcl1 | 11 | 25 | 0.908 | 0.009 | 1.96E+00 | 4.57E−161 | 8.09E−154 |
| Gata3 | 12 | 14 | 0.946 | 0.066 | 1.76E+00 | 1.57E−197 | 2.78E−190 |
| Hs3st1 | 12 | 17 | 0.846 | 0.003 | 1.70E+00 | 2.51E−189 | 4.44E−182 |
| Lmo4 | 12 | 18 | 0.988 | 0.089 | 2.52E+00 | 4.76E−262 | 8.43E−255 |
| Hs3st1 | 12 | 18 | 0.846 | 0.007 | 1.69E+00 | 2.17E−162 | 3.85E−155 |
| Lmo4 | 12 | 19 | 0.988 | 0.084 | 2.51E+00 | 1.10E−206 | 1.95E−199 |
| Gata3 | 12 | 20 | 0.946 | 0.076 | 1.70E+00 | 6.31E−171 | 1.12E−163 |
| Gata3 | 12 | 22 | 0.946 | 0.082 | 1.70E+00 | 2.26E−159 | 4.00E−152 |
| Trbc1 | 12 | 24 | 0.946 | 0.076 | 1.59E+00 | 9.97E−173 | 1.76E−165 |
| Gata3 | 12 | 24 | 0.946 | 0.031 | 1.81E+00 | 2.45E−167 | 4.34E−160 |
| Gata3 | 12 | 25 | 0.946 | 0.027 | 1.81E+00 | 1.05E−175 | 1.86E−168 |
| Trbc1 | 12 | 25 | 0.946 | 0.059 | 1.58E+00 | 1.28E−165 | 2.27E−158 |
| Trbc1 | 12 | 26 | 0.946 | 0.069 | 1.57E+00 | 1.27E−169 | 2.25E−162 |
| Gata3 | 12 | 26 | 0.946 | 0.028 | 1.77E+00 | 1.10E−163 | 1.94E−156 |
| Cd7 | 17 | 18 | 0.988 | 0.05 | 3.01E+00 | 2.24E−179 | 3.96E−172 |

TABLE 3
Differential expression analysis between cells from mice treated with OVA or PBS
in each cell type in PP (Table 3A) and LP (Table 3B) regions, related to FIG. 2.
A

| | | percentage of | percentage of | | | |
|---|---|---|---|---|---|---|
| | | expressing | expressing | | | |
| | cluster | cells in OVA- | cells in PBS- | LN (average | | p value, |
| gene | ID | treated group | treated group | fold change) | p value | adjusted |
| Tsc22d3 | 1 | 0.811 | 0.538 | 0.720 | 9.66E−129 | 7.61E−123 |
| Wdr89 | 1 | 0.983 | 0.941 | 0.366 | 3.01E−124 | 1.13E−118 |
| Txnip | 1 | 0.617 | 0.378 | 0.759 | 8.02E−98 | 1.05E−92 |
| Uba52 | 1 | 0.97 | 0.917 | 0.334 | 1.80E−65 | 1.42E−60 |
| Ddit4 | 1 | 0.348 | 0.209 | 0.532 | 7.46E−61 | 4.19E−56 |
| Bcl2 | 1 | 0.806 | 0.701 | 0.510 | 3.76E−55 | 1.64E−50 |
| Cd74 | 1 | 0.428 | 0.525 | −0.311 | 1.07E−49 | 3.67E−45 |
| Jund | 1 | 0.667 | 0.761 | −0.352 | 2.35E−45 | 6.62E−41 |
| Zfp36l2 | 1 | 0.566 | 0.302 | 0.837 | 1.47E−42 | 3.31E−38 |
| Il7r | 1 | 0.406 | 0.248 | 0.548 | 6.56E−39 | 1.03E−34 |
| Pik3ip1 | 1 | 0.495 | 0.361 | 0.321 | 2.62E−28 | 2.46E−24 |
| Hspa8 | 1 | 0.965 | 0.967 | −0.313 | 6.56E−25 | 4.61E−21 |
| Hsp90ab1 | 1 | 0.82 | 0.867 | −0.340 | 3.66E−23 | 2.34E−19 |
| Pdcd4 | 1 | 0.626 | 0.487 | 0.353 | 3.23E−22 | 1.93E−18 |
| Klf6 | 1 | 0.388 | 0.474 | −0.370 | 1.86E−20 | 9.96E−17 |
| Igha | 1 | 0.166 | 0.1 | 0.552 | 8.23E−20 | 4.26E−16 |
| Ifngr1 | 1 | 0.434 | 0.268 | 0.579 | 8.75E−19 | 3.92E−15 |
| Ltb | 1 | 0.826 | 0.847 | −0.221 | 6.06E−18 | 2.54E−14 |
| Dnaja1 | 1 | 0.551 | 0.652 | −0.413 | 8.11E−18 | 3.32E−14 |
| Iglc1 | 1 | 0.072 | 0.158 | −0.884 | 1.12E−16 | 4.05E−13 |
| Gramd3 | 1 | 0.386 | 0.222 | 0.593 | 1.12E−16 | 4.05E−13 |
| Klhl6 | 1 | 0.299 | 0.211 | 0.353 | 1.21E−16 | 4.37E−13 |
| DynIl1 | 1 | 0.64 | 0.721 | −0.268 | 1.48E−16 | 5.28E−13 |
| Rnaset2a | 1 | 0.583 | 0.437 | 0.311 | 1.65E−15 | 5.21E−12 |
| Ube2s | 1 | 0.472 | 0.361 | 0.296 | 1.79E−15 | 5.56E−12 |
| Ucp2 | 1 | 0.764 | 0.636 | 0.295 | 4.56E−15 | 1.35E−11 |
| Igkc | 1 | 0.531 | 0.601 | −0.321 | 5.17E−15 | 1.52E−11 |
| Grcc10 | 1 | 0.557 | 0.414 | 0.357 | 1.95E−14 | 5.45E−11 |
| Ubb | 1 | 0.989 | 0.983 | −0.168 | 2.36E−14 | 6.45E−11 |
| Srsf3 | 1 | 0.505 | 0.555 | −0.236 | 5.37E−14 | 1.44E−10 |
| VgIl4 | 1 | 0.274 | 0.322 | −0.350 | 2.14E−13 | 5.48E−10 |
| H2-Aa | 1 | 0.211 | 0.228 | −0.202 | 8.81E−13 | 2.04E−09 |
| Tnfrsf18 | 1 | 0.392 | 0.27 | 0.456 | 1.03E−12 | 2.35E−09 |
| Dnajb1 | 1 | 0.222 | 0.347 | −0.700 | 1.25E−12 | 2.85E−09 |
| Cd40lg | 1 | 0.138 | 0.194 | −0.506 | 2.21E−12 | 4.84E−09 |
| Tpm3 | 1 | 0.621 | 0.5 | 0.239 | 6.64E−12 | 1.38E−08 |
| Hsp90aa1 | 1 | 0.558 | 0.661 | −0.444 | 1.07E−10 | 1.97E−07 |
| Tnfsf8 | 1 | 0.181 | 0.099 | 0.471 | 2.28E−10 | 3.97E−07 |
| Ppp1r15a | 1 | 0.281 | 0.311 | −0.226 | 2.29E−10 | 3.98E−07 |
| Atf4 | 1 | 0.218 | 0.261 | −0.316 | 2.32E−10 | 4.01E−07 |
| Ms4a4b | 1 | 0.921 | 0.858 | 0.189 | 2.65E−10 | 4.50E−07 |
| Ahsa1 | 1 | 0.247 | 0.307 | −0.359 | 2.91E−10 | 4.92E−07 |
| Fos | 1 | 0.22 | 0.261 | −0.255 | 4.30E−10 | 7.12E−07 |
| Klhdc1 | 1 | 0.327 | 0.225 | 0.342 | 6.23E−10 | 1.00E−06 |
| Kbtbd11 | 1 | 0.314 | 0.219 | 0.355 | 1.78E−09 | 2.69E−06 |
| Neat1 | 1 | 0.274 | 0.3 | −0.269 | 1.80E−09 | 2.72E−06 |
| Nrip1 | 1 | 0.247 | 0.16 | 0.455 | 2.74E−09 | 4.01E−06 |
| Lrrc58 | 1 | 0.437 | 0.35 | 0.238 | 3.24E−09 | 4.69E−06 |
| Klhdc2 | 1 | 0.281 | 0.201 | 0.399 | 4.01E−09 | 5.75E−06 |
| Rcsd1 | 1 | 0.336 | 0.254 | 0.283 | 4.20E−09 | 6.01E−06 |
| Ptpn22 | 1 | 0.215 | 0.132 | 0.489 | 4.66E−09 | 6.61E−06 |
| Cacybp | 1 | 0.305 | 0.393 | −0.386 | 4.92E−09 | 6.94E−06 |
| Ier5 | 1 | 0.244 | 0.268 | −0.208 | 6.69E−09 | 9.22E−06 |
| Socs1 | 1 | 0.226 | 0.134 | 0.523 | 1.68E−08 | 2.16E−05 |
| Arl4c | 1 | 0.459 | 0.495 | −0.184 | 1.76E−08 | 2.26E−05 |
| Bcl11b | 1 | 0.431 | 0.474 | −0.226 | 2.69E−08 | 3.36E−05 |
| Hspe1 | 1 | 0.659 | 0.677 | −0.196 | 3.26E−08 | 4.03E−05 |
| Ctsc | 1 | 0.155 | 0.113 | 0.285 | 3.64E−08 | 4.48E−05 |
| Gstp1 | 1 | 0.332 | 0.258 | 0.204 | 5.44E−08 | 6.48E−05 |
| Acp5 | 1 | 0.414 | 0.325 | 0.201 | 6.23E−08 | 7.30E−05 |
| Tagap | 1 | 0.183 | 0.207 | −0.202 | 6.30E−08 | 7.35E−05 |
| Slbp | 1 | 0.129 | 0.164 | −0.301 | 1.11E−07 | 1.23E−04 |
| Ets1 | 1 | 0.625 | 0.519 | 0.186 | 1.19E−07 | 1.33E−04 |
| Rbm3 | 1 | 0.838 | 0.758 | 0.201 | 1.41E−07 | 1.54E−04 |
| Cetn3 | 1 | 0.259 | 0.188 | 0.240 | 2.79E−07 | 2.89E−04 |
| Cd3e | 1 | 0.891 | 0.815 | 0.170 | 3.56E−07 | 3.58E−04 |
| Bloc1s1 | 1 | 0.162 | 0.107 | 0.349 | 4.83E−07 | 4.76E−04 |
| Jun | 1 | 0.23 | 0.297 | −0.389 | 6.84E−07 | 6.50E−04 |
| Crlf3 | 1 | 0.594 | 0.469 | 0.317 | 7.03E−07 | 6.67E−04 |
| Ywhah | 1 | 0.377 | 0.408 | −0.167 | 1.38E−06 | 1.24E−03 |
| Cnp | 1 | 0.487 | 0.409 | 0.157 | 1.53E−06 | 1.37E−03 |
| Dusp2 | 1 | 0.367 | 0.292 | 0.319 | 1.56E−06 | 1.39E−03 |
| Slfn2 | 1 | 0.55 | 0.432 | 0.289 | 2.16E−06 | 1.88E−03 |
| Sfpq | 1 | 0.311 | 0.341 | −0.216 | 2.48E−06 | 2.14E−03 |
| 2810474O19Rik | 1 | 0.318 | 0.262 | 0.171 | 2.93E−06 | 2.47E−03 |
| Stip1 | 1 | 0.177 | 0.221 | −0.355 | 3.30E−06 | 2.76E−03 |
| Mfap1b | 1 | 0.157 | 0.123 | 0.161 | 4.60E−06 | 3.70E−03 |
| Tubb4b | 1 | 0.183 | 0.206 | −0.198 | 4.76E−06 | 3.80E−03 |
| Stt3b | 1 | 0.281 | 0.206 | 0.252 | 5.25E−06 | 4.14E−03 |
| Tmem50a | 1 | 0.595 | 0.485 | 0.195 | 6.05E−06 | 4.69E−03 |
| Sh3bp5 | 1 | 0.239 | 0.171 | 0.302 | 8.96E−06 | 6.65E−03 |
| Smad7 | 1 | 0.301 | 0.322 | −0.160 | 1.15E−05 | 8.28E−03 |
| Zfp36 | 1 | 0.196 | 0.223 | −0.174 | 1.54E−05 | 1.07E−02 |
| Atp1b3 | 1 | 0.692 | 0.595 | 0.188 | 1.56E−05 | 1.09E−02 |
| Sdhaf1 | 1 | 0.167 | 0.097 | 0.511 | 1.61E−05 | 1.12E−02 |
| Grb2 | 1 | 0.202 | 0.266 | −0.357 | 1.74E−05 | 1.20E−02 |
| Rhof | 1 | 0.195 | 0.214 | −0.180 | 1.98E−05 | 1.34E−02 |
| Rheb | 1 | 0.208 | 0.229 | −0.179 | 2.01E−05 | 1.36E−02 |
| H2-T23 | 1 | 0.252 | 0.173 | 0.288 | 3.34E−05 | 2.16E−02 |
| Fkbp4 | 1 | 0.273 | 0.315 | −0.251 | 4.39E−05 | 2.76E−02 |
| Zfos1 | 1 | 0.299 | 0.316 | −0.155 | 6.66E−05 | 3.94E−02 |
| Anapc16 | 1 | 0.199 | 0.144 | 0.279 | 7.73E−05 | 4.49E−02 |
| Ywhab | 1 | 0.328 | 0.358 | −0.172 | 8.66E−05 | 4.93E−02 |
| Irf1 | 1 | 0.155 | 0.187 | −0.208 | 8.82E−05 | 5.00E−02 |
| Zfp36l2 | 2 | 0.64 | 0.218 | 1.626 | 2.64E−112 | 4.65E−107 |
| Tsc22d3 | 2 | 0.806 | 0.526 | 0.816 | 3.02E−56 | 1.40E−51 |
| Sesn1 | 2 | 0.32 | 0.099 | 1.317 | 1.40E−32 | 1.62E−28 |
| Ptpn22 | 2 | 0.213 | 0.093 | 0.999 | 2.68E−21 | 1.51E−17 |
| Ddit4 | 2 | 0.216 | 0.126 | 0.714 | 5.00E−20 | 2.62E−16 |
| Fam107b | 2 | 0.53 | 0.334 | 0.653 | 7.67E−20 | 4.00E−16 |
| Iglc2 | 2 | 0.861 | 0.924 | −0.275 | 1.01E−17 | 4.06E−14 |
| Hs3st1 | 2 | 0.183 | 0.072 | 0.958 | 4.47E−16 | 1.49E−12 |
| Iglc3 | 2 | 0.86 | 0.905 | −0.196 | 9.41E−16 | 3.05E−12 |
| Pik3ip1 | 2 | 0.231 | 0.131 | 0.568 | 1.42E−15 | 4.49E−12 |
| Ccnd3 | 2 | 0.321 | 0.171 | 0.710 | 1.41E−14 | 4.00E−11 |
| Plaur | 2 | 0.273 | 0.093 | 1.207 | 2.91E−14 | 7.91E−11 |
| Pdcd4 | 2 | 0.426 | 0.32 | 0.482 | 1.82E−11 | 3.57E−08 |
| Btg1 | 2 | 0.996 | 0.99 | 0.251 | 5.67E−11 | 1.07E−07 |
| Cd69 | 2 | 0.502 | 0.367 | 0.462 | 7.31E−11 | 1.37E−07 |
| Swap70 | 2 | 0.326 | 0.373 | −0.259 | 1.59E−10 | 2.83E−07 |
| Srgn | 2 | 0.867 | 0.745 | 0.347 | 1.93E−10 | 3.39E−07 |
| Cd24a | 2 | 0.582 | 0.596 | −0.234 | 4.21E−10 | 6.98E−07 |
| Egln2 | 2 | 0.311 | 0.169 | 0.709 | 1.07E−09 | 1.67E−06 |
| Rsrp1 | 2 | 0.481 | 0.564 | −0.342 | 4.13E−08 | 5.03E−05 |
| Ltb | 2 | 0.837 | 0.853 | −0.251 | 5.43E−08 | 6.48E−05 |
| Cxcr4 | 2 | 0.671 | 0.477 | 0.491 | 7.26E−08 | 8.37E−05 |
| Sorl1 | 2 | 0.4 | 0.267 | 0.459 | 2.08E−07 | 2.21E−04 |
| Sla | 2 | 0.251 | 0.145 | 0.642 | 2.53E−07 | 2.64E−04 |
| Smad7 | 2 | 0.212 | 0.13 | 0.479 | 2.58E−07 | 2.68E−04 |
| Cd180 | 2 | 0.281 | 0.334 | −0.252 | 3.15E−07 | 3.23E−04 |
| Sdhaf1 | 2 | 0.31 | 0.228 | 0.473 | 3.64E−07 | 3.66E−04 |
| Txnip | 2 | 0.403 | 0.255 | 0.611 | 4.70E−07 | 4.65E−04 |
| Egr1 | 2 | 0.289 | 0.198 | 0.594 | 6.79E−07 | 6.46E−04 |
| Gpr171 | 2 | 0.445 | 0.272 | 0.554 | 7.35E−07 | 6.95E−04 |
| Smc6 | 2 | 0.673 | 0.58 | 0.212 | 8.29E−07 | 7.73E−04 |
| Malat1 | 2 | 1 | 0.999 | −0.189 | 1.92E−06 | 1.69E−03 |
| Ly6d | 2 | 0.253 | 0.339 | −0.495 | 2.27E−06 | 1.97E−03 |
| Sypl | 2 | 0.434 | 0.307 | 0.405 | 4.65E−06 | 3.73E−03 |
| Napsa | 2 | 0.749 | 0.638 | 0.242 | 4.95E−06 | 3.92E−03 |
| Acp5 | 2 | 0.411 | 0.313 | 0.289 | 5.31E−06 | 4.18E−03 |
| P2ry10 | 2 | 0.308 | 0.167 | 0.629 | 8.14E−06 | 6.11E−03 |
| Hhex | 2 | 0.277 | 0.309 | −0.177 | 1.12E−05 | 8.07E−03 |
| Il10ra | 2 | 0.155 | 0.081 | 0.643 | 1.96E−05 | 1.34E−02 |
| 1110059E24Rik | 2 | 0.313 | 0.204 | 0.447 | 2.73E−05 | 1.80E−02 |
| Scd1 | 2 | 0.48 | 0.345 | 0.338 | 2.75E−05 | 1.81E−02 |
| Jmjd1c | 2 | 0.222 | 0.177 | 0.252 | 3.83E−05 | 2.45E−02 |
| Stk17b | 2 | 0.77 | 0.63 | 0.315 | 3.85E−05 | 2.45E−02 |
| Cd47 | 2 | 0.587 | 0.464 | 0.328 | 4.50E−05 | 2.82E−02 |
| Btg2 | 2 | 0.767 | 0.632 | 0.450 | 5.96E−05 | 3.59E−02 |
| Zfand5 | 2 | 0.161 | 0.213 | −0.399 | 6.22E−05 | 3.72E−02 |
| Bend5 | 2 | 0.21 | 0.151 | 0.306 | 6.81E−05 | 4.02E−02 |
| Sgms1 | 2 | 0.263 | 0.159 | 0.537 | 8.69E−05 | 4.94E−02 |
| Jund | 3 | 0.82 | 0.94 | −0.733 | 2.95E−112 | 4.65E−107 |
| Id2 | 3 | 0.923 | 0.738 | 0.591 | 1.26E−87 | 1.42E−82 |
| Pim1 | 3 | 0.754 | 0.884 | −0.496 | 4.49E−72 | 4.42E−67 |
| Nfkbia | 3 | 0.801 | 0.905 | −0.415 | 5.12E−63 | 3.36E−58 |
| Klf4 | 3 | 0.19 | 0.407 | −1.015 | 1.81E−52 | 7.14E−48 |
| Tgif1 | 3 | 0.407 | 0.6 | −0.586 | 4.81E−50 | 1.72E−45 |
| Hk2 | 3 | 0.253 | 0.457 | −0.836 | 1.71E−49 | 5.61E−45 |
| Junb | 3 | 0.957 | 0.971 | −0.156 | 8.61E−48 | 2.71E−43 |
| Gimap1 | 3 | 0.793 | 0.61 | 0.500 | 1.66E−47 | 5.02E−43 |
| Dusp5 | 3 | 0.535 | 0.687 | −0.410 | 7.77E−45 | 2.04E−40 |
| Nr4a1 | 3 | 0.607 | 0.752 | −0.338 | 8.83E−43 | 2.05E−38 |
| Dusp1 | 3 | 0.628 | 0.769 | −0.335 | 5.48E−42 | 1.17E−37 |
| Sla | 3 | 0.745 | 0.528 | 0.626 | 2.36E−41 | 4.77E−37 |
| Nfkbiz | 3 | 0.425 | 0.586 | −0.520 | 7.49E−41 | 1.47E−36 |
| Prr7 | 3 | 0.176 | 0.343 | −0.936 | 9.56E−41 | 1.84E−36 |
| AY036118 | 3 | 0.388 | 0.411 | −0.405 | 1.72E−40 | 3.23E−36 |
| Mcpt1 | 3 | 0.19 | 0.036 | 1.670 | 2.37E−40 | 4.33E−36 |
| Defa24 | 3 | 0.04 | 0.178 | −1.549 | 5.34E−40 | 9.56E−36 |
| Ifrd1 | 3 | 0.255 | 0.436 | −0.719 | 1.46E−39 | 2.55E−35 |
| Kdm6b | 3 | 0.291 | 0.454 | −0.672 | 3.57E−39 | 6.12E−35 |
| Ubb | 3 | 0.996 | 0.999 | −0.215 | 3.92E−39 | 6.56E−35 |
| Fam110a | 3 | 0.22 | 0.394 | −0.748 | 4.57E−38 | 6.92E−34 |
| Gadd45b | 3 | 0.533 | 0.698 | −0.196 | 2.97E−37 | 4.33E−33 |
| Maff | 3 | 0.435 | 0.573 | −0.425 | 2.69E−32 | 3.07E−28 |
| Bhlhe40 | 3 | 0.635 | 0.734 | −0.329 | 1.81E−31 | 2.00E−27 |
| Ppp1r15a | 3 | 0.312 | 0.473 | −0.550 | 2.34E−31 | 2.55E−27 |
| Tnfaip3 | 3 | 0.453 | 0.575 | −0.311 | 4.47E−31 | 4.82E−27 |
| Txnip | 3 | 0.222 | 0.08 | 1.243 | 2.01E−30 | 2.09E−26 |
| Vps37b | 3 | 0.551 | 0.657 | −0.438 | 4.05E−30 | 4.09E−26 |
| Lmna | 3 | 0.101 | 0.226 | −1.191 | 5.81E−30 | 5.79E−26 |
| Gimap6 | 3 | 0.841 | 0.722 | 0.343 | 1.67E−29 | 1.63E−25 |
| Fosb | 3 | 0.272 | 0.413 | −0.583 | 1.90E−29 | 1.82E−25 |
| Il22 | 3 | 0.188 | 0.33 | −0.772 | 4.09E−28 | 3.79E−24 |
| Cd69 | 3 | 0.68 | 0.767 | −0.330 | 7.25E−28 | 6.42E−24 |
| Bcl2 | 3 | 0.679 | 0.535 | 0.594 | 2.16E−27 | 1.83E−23 |
| Irf7 | 3 | 0.155 | 0.294 | −0.904 | 3.28E−26 | 2.56E−22 |
| Uhrf2 | 3 | 0.495 | 0.624 | −0.452 | 3.96E−26 | 3.03E−22 |
| Nfkb1 | 3 | 0.352 | 0.463 | −0.558 | 6.87E−26 | 5.15E−22 |
| Igha | 3 | 0.477 | 0.282 | 0.516 | 8.76E−26 | 6.45E−22 |
| Nfkbid | 3 | 0.316 | 0.454 | −0.489 | 1.85E−24 | 1.28E−20 |
| Dok1 | 3 | 0.367 | 0.198 | 0.611 | 4.60E−24 | 3.09E−20 |
| Pnrc1 | 3 | 0.752 | 0.826 | −0.247 | 1.44E−23 | 9.52E−20 |
| Rel | 3 | 0.129 | 0.253 | −0.749 | 2.98E−23 | 1.93E−19 |
| Litaf | 3 | 0.246 | 0.372 | −0.544 | 4.98E−23 | 3.16E−19 |
| Gimap9 | 3 | 0.472 | 0.304 | 0.525 | 2.44E−22 | 1.48E−18 |
| Nmrk1 | 3 | 0.092 | 0.204 | −0.763 | 6.05E−22 | 3.55E−18 |
| Atf3 | 3 | 0.086 | 0.19 | −0.803 | 6.89E−22 | 4.02E−18 |
| Coq10b | 3 | 0.268 | 0.384 | −0.483 | 7.50E−21 | 4.07E−17 |
| Taf10 | 3 | 0.26 | 0.402 | −0.563 | 8.81E−20 | 4.50E−16 |
| Gimap5 | 3 | 0.579 | 0.431 | 0.446 | 8.97E−20 | 4.54E−16 |
| Sik1 | 3 | 0.17 | 0.281 | −0.640 | 9.96E−20 | 4.99E−16 |
| Btg1 | 3 | 0.977 | 0.978 | −0.163 | 1.28E−19 | 6.33E−16 |
| Tsc22d3 | 3 | 0.514 | 0.347 | 0.622 | 1.43E−19 | 6.97E−16 |
| Ikzf2 | 3 | 0.663 | 0.742 | −0.369 | 3.39E−19 | 1.60E−15 |
| Rsrp1 | 3 | 0.623 | 0.492 | 0.268 | 3.47E−19 | 1.62E−15 |
| Nr4a3 | 3 | 0.197 | 0.3 | −0.600 | 3.69E−19 | 1.72E−15 |
| Ahr | 3 | 0.538 | 0.397 | 0.447 | 5.05E−19 | 2.34E−15 |
| Rgs2 | 3 | 0.535 | 0.633 | −0.297 | 5.75E−19 | 2.62E−15 |
| Cdkn1a | 3 | 0.082 | 0.177 | −0.870 | 8.51E−19 | 3.83E−15 |
| Phlda1 | 3 | 0.248 | 0.38 | −0.522 | 2.57E−18 | 1.12E−14 |
| Ncoa7 | 3 | 0.755 | 0.627 | 0.345 | 2.84E−18 | 1.23E−14 |
| D16Ertd472e | 3 | 0.223 | 0.311 | −0.484 | 5.45E−18 | 2.29E−14 |
| Ffar2 | 3 | 0.752 | 0.642 | 0.294 | 7.23E−18 | 2.98E−14 |
| Egr1 | 3 | 0.192 | 0.294 | −0.541 | 1.27E−17 | 5.02E−14 |
| Zc3h12a | 3 | 0.237 | 0.338 | −0.483 | 1.53E−17 | 6.00E−14 |
| Rasl11a | 3 | 0.346 | 0.216 | 0.766 | 1.77E−17 | 6.86E−14 |
| Cited4 | 3 | 0.479 | 0.346 | 0.361 | 4.36E−17 | 1.65E−13 |
| Serpinb9 | 3 | 0.367 | 0.448 | −0.428 | 5.58E−17 | 2.08E−13 |
| Ier5l | 3 | 0.067 | 0.153 | −1.214 | 9.93E−17 | 3.62E−13 |
| Mir142hg | 3 | 0.077 | 0.161 | −0.709 | 1.29E−16 | 4.60E−13 |
| Nabp1 | 3 | 0.544 | 0.591 | −0.340 | 1.87E−16 | 6.64E−13 |
| Zfp36l2 | 3 | 0.523 | 0.364 | 0.563 | 3.72E−16 | 1.27E−12 |
| Igkc | 3 | 0.661 | 0.527 | 0.304 | 4.32E−16 | 1.45E−12 |
| Ubc | 3 | 0.347 | 0.457 | −0.378 | 1.66E−15 | 5.22E−12 |
| Bcl2a1d | 3 | 0.407 | 0.488 | −0.357 | 2.09E−15 | 6.44E−12 |
| Slc16a6 | 3 | 0.176 | 0.267 | −0.490 | 4.79E−15 | 1.41E−11 |
| Crem | 3 | 0.291 | 0.372 | −0.456 | 5.75E−15 | 1.68E−11 |
| Cd74 | 3 | 0.43 | 0.53 | −0.422 | 1.31E−14 | 3.73E−11 |
| Il2rg | 3 | 0.76 | 0.791 | −0.199 | 1.95E−14 | 5.45E−11 |
| Furin | 3 | 0.367 | 0.454 | −0.360 | 6.03E−14 | 1.62E−10 |
| Fosl2 | 3 | 0.337 | 0.427 | −0.340 | 6.29E−14 | 1.67E−10 |
| Slfn2 | 3 | 0.435 | 0.311 | 0.459 | 7.64E−14 | 2.01E−10 |
| Icos | 3 | 0.43 | 0.502 | −0.325 | 1.02E−13 | 2.67E−10 |
| Emb | 3 | 0.903 | 0.922 | −0.182 | 1.09E−13 | 2.83E−10 |
| Nr4a2 | 3 | 0.112 | 0.186 | −0.662 | 1.32E−13 | 3.42E−10 |
| Cd83 | 3 | 0.211 | 0.303 | −0.369 | 1.43E−13 | 3.68E−10 |
| Traf1 | 3 | 0.557 | 0.627 | −0.266 | 3.07E−13 | 7.70E−10 |
| Alyref | 3 | 0.127 | 0.23 | −0.688 | 3.57E−13 | 8.86E−10 |
| Dusp2 | 3 | 0.313 | 0.194 | 0.870 | 7.85E−13 | 1.86E−09 |
| Sqstm1 | 3 | 0.365 | 0.456 | −0.328 | 8.72E−13 | 2.03E−09 |
| Rora | 3 | 0.734 | 0.775 | −0.228 | 1.57E−12 | 3.51E−09 |
| Gnai2 | 3 | 0.583 | 0.673 | −0.341 | 1.87E−12 | 4.17E−09 |
| Il7r | 3 | 0.934 | 0.891 | 0.206 | 3.05E−12 | 6.56E−09 |
| Serpinb6b | 3 | 0.421 | 0.497 | −0.223 | 4.37E−12 | 9.26E−09 |
| Ramp3 | 3 | 0.316 | 0.37 | −0.470 | 9.85E−12 | 2.00E−08 |
| Ptp4a2 | 3 | 0.538 | 0.618 | −0.235 | 1.77E−11 | 3.51E−08 |
| Gna13 | 3 | 0.394 | 0.45 | −0.291 | 1.92E−11 | 3.76E−08 |
| Sla2 | 3 | 0.295 | 0.19 | 0.465 | 2.62E−11 | 5.11E−08 |
| Slpr4 | 3 | 0.182 | 0.097 | 0.601 | 6.96E−11 | 1.31E−07 |
| Cish | 3 | 0.39 | 0.287 | 0.423 | 9.27E−11 | 1.72E−07 |
| Neat1 | 3 | 0.46 | 0.349 | 0.291 | 1.37E−10 | 2.45E−07 |
| Fcer1g | 3 | 0.914 | 0.895 | 0.188 | 2.11E−10 | 3.69E−07 |
| Tagap | 3 | 0.277 | 0.352 | −0.196 | 2.30E−10 | 3.98E−07 |
| Rnf19b | 3 | 0.287 | 0.351 | −0.411 | 4.10E−10 | 6.82E−07 |
| 2010300C02Rik | 3 | 0.509 | 0.407 | 0.315 | 4.65E−10 | 7.65E−07 |
| Icam1 | 3 | 0.144 | 0.219 | −0.479 | 4.80E−10 | 7.88E−07 |
| Amica1 | 3 | 0.405 | 0.306 | 0.371 | 5.80E−10 | 9.38E−07 |
| Odc1 | 3 | 0.227 | 0.284 | −0.409 | 6.27E−10 | 1.01E−06 |
| Actr3 | 3 | 0.72 | 0.67 | 0.227 | 8.43E−10 | 1.34E−06 |
| Ppp2ca | 3 | 0.307 | 0.405 | −0.395 | 1.04E−09 | 1.63E−06 |
| Klrk1 | 3 | 0.733 | 0.75 | −0.160 | 1.05E−09 | 1.65E−06 |
| Hist1h1c | 3 | 0.3 | 0.204 | 0.664 | 1.05E−09 | 1.65E−06 |
| Sec11a | 3 | 0.441 | 0.351 | 0.300 | 1.14E−09 | 1.78E−06 |
| Csrnp1 | 3 | 0.258 | 0.326 | −0.236 | 1.25E−09 | 1.92E−06 |
| Nrip1 | 3 | 0.627 | 0.673 | −0.162 | 1.31E−09 | 2.01E−06 |
| Rilpl2 | 3 | 0.284 | 0.183 | 0.478 | 2.33E−09 | 3.46E−06 |
| Rinl | 3 | 0.624 | 0.521 | 0.233 | 2.45E−09 | 3.61E−06 |
| H2-Q7 | 3 | 0.393 | 0.459 | −0.237 | 2.58E−09 | 3.78E−06 |
| Ptpn7 | 3 | 0.355 | 0.266 | 0.326 | 3.15E−09 | 4.57E−06 |
| Neurl3 | 3 | 0.446 | 0.516 | −0.209 | 5.65E−09 | 7.93E−06 |
| Pdrg1 | 3 | 0.265 | 0.174 | 0.377 | 6.42E−09 | 8.88E−06 |
| Rnf125 | 3 | 0.099 | 0.158 | −0.583 | 6.81E−09 | 9.36E−06 |
| Chmp4b | 3 | 0.356 | 0.432 | −0.295 | 6.87E−09 | 9.42E−06 |
| Cd160 | 3 | 0.564 | 0.449 | 0.275 | 8.12E−09 | 1.10E−05 |
| Ifi47 | 3 | 0.265 | 0.172 | 0.465 | 8.87E−09 | 1.19E−05 |
| Arpc5l | 3 | 0.353 | 0.261 | 0.341 | 9.63E−09 | 1.29E−05 |
| Ifi203 | 3 | 0.47 | 0.376 | 0.352 | 1.02E−08 | 1.35E−05 |
| Stom | 3 | 0.165 | 0.091 | 0.580 | 1.15E−08 | 1.52E−05 |
| Cytip | 3 | 0.516 | 0.551 | −0.185 | 1.60E−08 | 2.07E−05 |
| Rab19 | 3 | 0.178 | 0.11 | 0.484 | 2.36E−08 | 2.99E−05 |
| Kcnk1 | 3 | 0.45 | 0.35 | 0.325 | 2.49E−08 | 3.12E−05 |
| Ccrl2 | 3 | 0.146 | 0.204 | −0.395 | 2.92E−08 | 3.64E−05 |
| Jagn1 | 3 | 0.156 | 0.087 | 0.602 | 3.06E−08 | 3.80E−05 |
| Orai1 | 3 | 0.293 | 0.354 | −0.265 | 3.72E−08 | 4.57E−05 |
| Amd1 | 3 | 0.169 | 0.232 | −0.397 | 3.85E−08 | 4.72E−05 |
| Dbp | 3 | 0.188 | 0.126 | 0.465 | 4.22E−08 | 5.12E−05 |
| Gimap7 | 3 | 0.598 | 0.519 | 0.236 | 4.58E−08 | 5.52E−05 |
| Nfil3 | 3 | 0.154 | 0.21 | −0.381 | 4.99E−08 | 5.98E−05 |
| Cmtm6 | 3 | 0.27 | 0.18 | 0.417 | 5.69E−08 | 6.77E−05 |
| Tmem243 | 3 | 0.246 | 0.315 | −0.347 | 6.06E−08 | 7.14E−05 |
| Gimap3 | 3 | 0.905 | 0.859 | 0.189 | 6.58E−08 | 7.66E−05 |
| Ptger2 | 3 | 0.204 | 0.139 | 0.460 | 6.94E−08 | 8.05E−05 |
| Fam195b | 3 | 0.287 | 0.201 | 0.360 | 7.37E−08 | 8.47E−05 |
| Asb2 | 3 | 0.551 | 0.469 | 0.246 | 8.68E−08 | 9.91E−05 |
| Tspan32 | 3 | 0.178 | 0.108 | 0.515 | 8.87E−08 | 1.01E−04 |
| Frmd4b | 3 | 0.169 | 0.23 | −0.389 | 9.08E−08 | 1.03E−04 |
| Tob2 | 3 | 0.253 | 0.306 | −0.243 | 9.74E−08 | 1.10E−04 |
| Ssna1 | 3 | 0.299 | 0.21 | 0.352 | 1.38E−07 | 1.52E−04 |
| Klf2 | 3 | 0.272 | 0.327 | −0.271 | 1.54E−07 | 1.69E−04 |
| Tvp23b | 3 | 0.17 | 0.095 | 0.546 | 1.78E−07 | 1.93E−04 |
| Sdhd | 3 | 0.276 | 0.186 | 0.419 | 2.51E−07 | 2.63E−04 |
| Tbx21 | 3 | 0.169 | 0.109 | 0.524 | 2.57E−07 | 2.68E−04 |
| Gem | 3 | 0.561 | 0.61 | −0.167 | 2.98E−07 | 3.07E−04 |
| Map3k14 | 3 | 0.155 | 0.217 | −0.422 | 3.09E−07 | 3.18E−04 |
| Sat1 | 3 | 0.665 | 0.599 | 0.179 | 3.33E−07 | 3.38E−04 |
| Tomm5 | 3 | 0.372 | 0.282 | 0.314 | 3.78E−07 | 3.79E−04 |
| 4921524J17Rik | 3 | 0.153 | 0.091 | 0.521 | 4.49E−07 | 4.45E−04 |
| Prex1 | 3 | 0.365 | 0.271 | 0.372 | 4.96E−07 | 4.86E−04 |
| Trpc4ap | 3 | 0.344 | 0.263 | 0.320 | 5.24E−07 | 5.11E−04 |
| Ube2g2 | 3 | 0.252 | 0.172 | 0.369 | 5.59E−07 | 5.41E−04 |
| Gimap8 | 3 | 0.238 | 0.151 | 0.472 | 5.81E−07 | 5.61E−04 |
| Smap2 | 3 | 0.369 | 0.442 | −0.258 | 6.66E−07 | 6.36E−04 |
| Kit | 3 | 0.411 | 0.458 | −0.204 | 6.69E−07 | 6.38E−04 |
| Tifa | 3 | 0.196 | 0.124 | 0.459 | 7.52E−07 | 7.10E−04 |
| P2ry10 | 3 | 0.261 | 0.318 | −0.229 | 7.55E−07 | 7.11E−04 |
| Ccnl1 | 3 | 0.379 | 0.44 | −0.180 | 8.06E−07 | 7.54E−04 |
| Tox | 3 | 0.253 | 0.171 | 0.492 | 8.57E−07 | 7.97E−04 |
| Mdfic | 3 | 0.437 | 0.345 | 0.292 | 1.03E−06 | 9.48E−04 |
| Serpinb1a | 3 | 0.768 | 0.784 | −0.178 | 1.09E−06 | 9.93E−04 |
| Rlim | 3 | 0.148 | 0.199 | −0.386 | 1.30E−06 | 1.18E−03 |
| Timm10b | 3 | 0.351 | 0.254 | 0.295 | 1.36E−06 | 1.22E−03 |
| F2r | 3 | 0.355 | 0.412 | −0.242 | 1.52E−06 | 1.35E−03 |
| Tgoln1 | 3 | 0.262 | 0.322 | −0.234 | 1.71E−06 | 1.51E−03 |
| Gpcpd1 | 3 | 0.187 | 0.246 | −0.328 | 1.83E−06 | 1.62E−03 |
| Bcl2a1b | 3 | 0.15 | 0.194 | −0.350 | 2.38E−06 | 2.06E−03 |
| VgIl4 | 3 | 0.24 | 0.29 | −0.332 | 2.43E−06 | 2.09E−03 |
| Ccdc115 | 3 | 0.218 | 0.151 | 0.408 | 2.71E−06 | 2.31E−03 |
| Arl6ip5 | 3 | 0.476 | 0.388 | 0.229 | 2.79E−06 | 2.37E−03 |
| Ddx3x | 3 | 0.353 | 0.403 | −0.175 | 2.88E−06 | 2.43E−03 |
| Polr2a | 3 | 0.209 | 0.26 | −0.277 | 3.08E−06 | 2.59E−03 |
| Gcnt2 | 3 | 0.258 | 0.305 | −0.265 | 3.87E−06 | 3.16E−03 |
| Ahcyl2 | 3 | 0.623 | 0.551 | 0.192 | 4.48E−06 | 3.61E−03 |
| H2-Eb1 | 3 | 0.162 | 0.212 | −0.312 | 4.63E−06 | 3.72E−03 |
| Dusp4 | 3 | 0.158 | 0.211 | −0.322 | 4.87E−06 | 3.87E−03 |
| Oard1 | 3 | 0.176 | 0.122 | 0.412 | 5.55E−06 | 4.36E−03 |
| Ormdl3 | 3 | 0.195 | 0.129 | 0.363 | 6.00E−06 | 4.66E−03 |
| Gpr171 | 3 | 0.382 | 0.279 | 0.299 | 6.36E−06 | 4.91E−03 |
| Zc3hav1 | 3 | 0.223 | 0.271 | −0.209 | 6.73E−06 | 5.15E−03 |
| Ezr | 3 | 0.322 | 0.365 | −0.189 | 6.82E−06 | 5.21E−03 |
| Clk1 | 3 | 0.55 | 0.585 | −0.156 | 7.20E−06 | 5.48E−03 |
| Zkscan3 | 3 | 0.157 | 0.105 | 0.422 | 7.50E−06 | 5.68E−03 |
| Klrc1 | 3 | 0.225 | 0.156 | 0.505 | 8.30E−06 | 6.21E−03 |
| Plekhg1 | 3 | 0.255 | 0.312 | −0.320 | 8.53E−06 | 6.37E−03 |
| Tbc1d10c | 3 | 0.471 | 0.415 | 0.204 | 8.69E−06 | 6.48E−03 |
| Gjb2 | 3 | 0.209 | 0.263 | −0.286 | 8.75E−06 | 6.52E−03 |
| Rnf166 | 3 | 0.351 | 0.271 | 0.250 | 8.89E−06 | 6.61E−03 |
| Tcrg-C1 | 3 | 0.671 | 0.607 | 0.219 | 9.69E−06 | 7.15E−03 |
| Clec2d | 3 | 0.243 | 0.172 | 0.363 | 9.72E−06 | 7.17E−03 |
| Snx20 | 3 | 0.194 | 0.129 | 0.403 | 1.02E−05 | 7.47E−03 |
| Rnf114 | 3 | 0.251 | 0.188 | 0.288 | 1.04E−05 | 7.63E−03 |
| Smim11 | 3 | 0.365 | 0.286 | 0.307 | 1.10E−05 | 8.01E−03 |
| Ptprcap | 3 | 0.818 | 0.753 | 0.186 | 1.17E−05 | 8.39E−03 |
| Timm13 | 3 | 0.556 | 0.475 | 0.211 | 1.23E−05 | 8.79E−03 |
| Ndel1 | 3 | 0.167 | 0.212 | −0.336 | 1.31E−05 | 9.33E−03 |
| Supt4a | 3 | 0.672 | 0.601 | 0.195 | 1.37E−05 | 9.70E−03 |
| Emd | 3 | 0.293 | 0.343 | −0.185 | 1.41E−05 | 9.94E−03 |
| Rnf5 | 3 | 0.194 | 0.129 | 0.419 | 1.44E−05 | 1.01E−02 |
| BC031181 | 3 | 0.502 | 0.543 | −0.166 | 1.60E−05 | 1.12E−02 |
| Mxd1 | 3 | 0.269 | 0.32 | −0.211 | 1.63E−05 | 1.13E−02 |
| Rgs3 | 3 | 0.25 | 0.178 | 0.356 | 1.79E−05 | 1.23E−02 |
| Gatad1 | 3 | 0.153 | 0.208 | −0.322 | 1.83E−05 | 1.26E−02 |
| Stat4 | 3 | 0.297 | 0.321 | −0.222 | 1.92E−05 | 1.31E−02 |
| Tmx1 | 3 | 0.199 | 0.153 | 0.318 | 2.53E−05 | 1.69E−02 |
| Nudt16l1 | 3 | 0.183 | 0.121 | 0.403 | 2.76E−05 | 1.82E−02 |
| Gnb1 | 3 | 0.395 | 0.452 | −0.269 | 2.88E−05 | 1.89E−02 |
| Fam184b | 3 | 0.232 | 0.183 | 0.317 | 3.00E−05 | 1.97E−02 |
| Tuba1c | 3 | 0.125 | 0.162 | −0.301 | 3.03E−05 | 1.99E−02 |
| Pten | 3 | 0.205 | 0.263 | −0.336 | 3.04E−05 | 1.99E−02 |
| Klrb1f | 3 | 0.413 | 0.338 | 0.210 | 3.35E−05 | 2.17E−02 |
| Fyn | 3 | 0.169 | 0.223 | −0.338 | 3.65E−05 | 2.34E−02 |
| Bloc1s2 | 3 | 0.159 | 0.106 | 0.414 | 3.90E−05 | 2.49E−02 |
| Commd6 | 3 | 0.238 | 0.171 | 0.343 | 4.08E−05 | 2.58E−02 |
| Tuba1a | 3 | 0.214 | 0.258 | −0.209 | 4.09E−05 | 2.59E−02 |
| Ppt2 | 3 | 0.389 | 0.332 | 0.224 | 4.28E−05 | 2.70E−02 |
| Scn1b | 3 | 0.138 | 0.186 | −0.340 | 4.92E−05 | 3.05E−02 |
| Birc3 | 3 | 0.297 | 0.337 | −0.173 | 4.99E−05 | 3.09E−02 |
| Skil | 3 | 0.198 | 0.247 | −0.266 | 5.04E−05 | 3.11E−02 |
| Tmem261 | 3 | 0.211 | 0.142 | 0.365 | 5.28E−05 | 3.24E−02 |
| Ube2s | 3 | 0.433 | 0.47 | −0.154 | 5.28E−05 | 3.24E−02 |
| 4930523C07Rik | 3 | 0.386 | 0.426 | −0.187 | 5.83E−05 | 3.53E−02 |
| C4b | 3 | 0.321 | 0.26 | 0.336 | 5.89E−05 | 3.56E−02 |
| Traf2 | 3 | 0.19 | 0.137 | 0.321 | 5.89E−05 | 3.56E−02 |
| Rbm4b | 3 | 0.114 | 0.159 | −0.349 | 5.90E−05 | 3.56E−02 |
| 4930453N24Rik | 3 | 0.237 | 0.183 | 0.309 | 5.97E−05 | 3.59E−02 |
| Triap1 | 3 | 0.167 | 0.116 | 0.346 | 6.00E−05 | 3.60E−02 |
| Ptpn22 | 3 | 0.603 | 0.508 | 0.266 | 6.06E−05 | 3.64E−02 |
| Il18r1 | 3 | 0.626 | 0.561 | 0.165 | 6.22E−05 | 3.72E−02 |
| Podnl1 | 3 | 0.66 | 0.594 | 0.171 | 6.29E−05 | 3.75E−02 |
| Nt5e | 3 | 0.216 | 0.164 | 0.329 | 7.59E−05 | 4.42E−02 |
| Pnrc2 | 3 | 0.191 | 0.148 | 0.224 | 7.74E−05 | 4.49E−02 |
| Ppp1r3b | 3 | 0.16 | 0.195 | −0.172 | 8.39E−05 | 4.81E−02 |
| Jund | 4 | 0.888 | 0.978 | −0.773 | 4.29E−124 | 1.13E−118 |
| Klf4 | 4 | 0.284 | 0.583 | −1.032 | 4.37E−69 | 3.82E−64 |
| Junb | 4 | 0.957 | 0.98 | −0.249 | 7.45E−65 | 5.33E−60 |
| Lmna | 4 | 0.241 | 0.503 | −1.267 | 2.12E−60 | 1.11E−55 |
| Ncoa7 | 4 | 0.899 | 0.725 | 0.635 | 3.60E−60 | 1.77E−55 |
| Dusp1 | 4 | 0.657 | 0.816 | −0.419 | 4.71E−45 | 1.28E−40 |
| Pim1 | 4 | 0.688 | 0.839 | −0.372 | 8.88E−45 | 2.26E−40 |
| Rasl11a | 4 | 0.737 | 0.504 | 0.766 | 2.69E−42 | 5.89E−38 |
| Fam110a | 4 | 0.453 | 0.62 | −0.604 | 4.10E−39 | 6.72E−35 |
| Sla | 4 | 0.819 | 0.608 | 0.624 | 1.04E−36 | 1.50E−32 |
| B930036N10Rik | 4 | 0.377 | 0.159 | 0.957 | 9.56E−34 | 1.26E−29 |
| Ifrd1 | 4 | 0.37 | 0.551 | −0.531 | 1.21E−33 | 1.50E−29 |
| Ubb | 4 | 0.997 | 1 | −0.219 | 1.22E−33 | 1.50E−29 |
| Nfkbia | 4 | 0.882 | 0.937 | −0.305 | 3.74E−33 | 4.46E−29 |
| Gimap1 | 4 | 0.735 | 0.537 | 0.488 | 6.73E−33 | 7.91E−29 |
| Maff | 4 | 0.554 | 0.688 | −0.378 | 8.53E−32 | 9.60E−28 |
| Fosb | 4 | 0.561 | 0.668 | −0.453 | 4.03E−30 | 4.09E−26 |
| Bcl2 | 4 | 0.912 | 0.804 | 0.403 | 1.05E−29 | 1.04E−25 |
| Kdm6b | 4 | 0.324 | 0.491 | −0.629 | 2.70E−27 | 2.26E−23 |
| Emb | 4 | 0.822 | 0.864 | −0.325 | 3.20E−27 | 2.65E−23 |
| Ahr | 4 | 0.448 | 0.264 | 0.772 | 1.57E−26 | 1.25E−22 |
| Prr7 | 4 | 0.192 | 0.354 | −0.719 | 1.91E−26 | 1.51E−22 |
| Ffar2 | 4 | 0.669 | 0.487 | 0.537 | 3.71E−26 | 2.86E−22 |
| AY036118 | 4 | 0.316 | 0.415 | −0.746 | 8.43E−26 | 6.26E−22 |
| Rora | 4 | 0.674 | 0.77 | −0.391 | 1.57E−25 | 1.14E−21 |
| Hspa1a | 4 | 0.308 | 0.173 | 0.397 | 5.71E−25 | 4.05E−21 |
| Pnrc1 | 4 | 0.805 | 0.864 | −0.272 | 1.65E−24 | 1.15E−20 |
| Phlda1 | 4 | 0.292 | 0.469 | −0.447 | 1.97E−22 | 1.21E−18 |
| Gimap6 | 4 | 0.721 | 0.573 | 0.417 | 3.67E−22 | 2.17E−18 |
| Cd83 | 4 | 0.504 | 0.632 | −0.288 | 5.17E−21 | 2.83E−17 |
| Igha | 4 | 0.463 | 0.305 | 0.385 | 8.55E−20 | 4.40E−16 |
| Nmrk1 | 4 | 0.262 | 0.395 | −0.442 | 2.64E−19 | 1.25E−15 |
| Rilpl2 | 4 | 0.356 | 0.189 | 0.762 | 5.33E−19 | 2.45E−15 |
| Tnfaip3 | 4 | 0.51 | 0.62 | −0.220 | 5.42E−19 | 2.48E−15 |
| Dusp5 | 4 | 0.443 | 0.57 | −0.400 | 5.87E−19 | 2.66E−15 |
| Actg1 | 4 | 0.938 | 0.964 | −0.209 | 1.09E−18 | 4.84E−15 |
| Id2 | 4 | 0.921 | 0.805 | 0.376 | 3.64E−18 | 1.56E−14 |
| Odc1 | 4 | 0.786 | 0.816 | −0.296 | 4.30E−18 | 1.83E−14 |
| Ppp1r15a | 4 | 0.312 | 0.424 | −0.441 | 7.06E−18 | 2.93E−14 |
| Igkc | 4 | 0.718 | 0.569 | 0.306 | 8.21E−18 | 3.35E−14 |
| Avpi1 | 4 | 0.203 | 0.313 | −0.575 | 1.09E−17 | 4.34E−14 |
| Vps37b | 4 | 0.489 | 0.593 | −0.376 | 1.12E−17 | 4.47E−14 |
| Gem | 4 | 0.727 | 0.813 | −0.217 | 5.78E−17 | 2.15E−13 |
| Nfkb1 | 4 | 0.415 | 0.523 | −0.507 | 4.11E−16 | 1.40E−12 |
| Atf3 | 4 | 0.19 | 0.308 | −0.487 | 5.16E−16 | 1.71E−12 |
| Traf1 | 4 | 0.59 | 0.663 | −0.296 | 6.90E−16 | 2.26E−12 |
| Rrad | 4 | 0.294 | 0.412 | −0.763 | 1.10E−15 | 3.50E−12 |
| Sertad1 | 4 | 0.091 | 0.185 | −0.838 | 1.72E−15 | 5.37E−12 |
| Tnfsf14 | 4 | 0.172 | 0.074 | 1.043 | 3.02E−15 | 9.14E−12 |
| Sqstm1 | 4 | 0.467 | 0.557 | −0.345 | 3.13E−15 | 9.45E−12 |
| Crem | 4 | 0.343 | 0.431 | −0.494 | 4.12E−15 | 1.23E−11 |
| Hk2 | 4 | 0.184 | 0.286 | −0.731 | 4.51E−15 | 1.34E−11 |
| Neurl3 | 4 | 0.301 | 0.412 | −0.324 | 1.14E−14 | 3.28E−11 |
| Defa24 | 4 | 0.076 | 0.169 | −0.827 | 1.37E−14 | 3.89E−11 |
| Ier5l | 4 | 0.09 | 0.202 | −1.300 | 1.65E−14 | 4.66E−11 |
| Ubc | 4 | 0.417 | 0.508 | −0.308 | 2.10E−14 | 5.80E−11 |
| Zc3h12a | 4 | 0.336 | 0.43 | −0.358 | 3.83E−14 | 1.04E−10 |
| Ube2s | 4 | 0.511 | 0.581 | −0.264 | 3.26E−13 | 8.14E−10 |
| H2-Q7 | 4 | 0.321 | 0.439 | −0.400 | 4.20E−13 | 1.03E−09 |
| Tgif1 | 4 | 0.522 | 0.585 | −0.211 | 4.99E−13 | 1.21E−09 |
| Uhrf2 | 4 | 0.677 | 0.743 | −0.213 | 5.36E−13 | 1.30E−09 |
| Gnai2 | 4 | 0.596 | 0.701 | −0.411 | 8.35E−13 | 1.96E−09 |
| Pcyt1a | 4 | 0.101 | 0.19 | −0.727 | 9.33E−13 | 2.16E−09 |
| Il22 | 4 | 0.3 | 0.4 | −0.341 | 1.71E−12 | 3.83E−09 |
| Pdrg1 | 4 | 0.255 | 0.154 | 0.546 | 2.24E−12 | 4.89E−09 |
| Cish | 4 | 0.393 | 0.255 | 0.530 | 2.75E−12 | 5.93E−09 |
| Ccng2 | 4 | 0.257 | 0.316 | −0.410 | 1.27E−11 | 2.56E−08 |
| Nfkbiz | 4 | 0.422 | 0.524 | −0.247 | 1.63E−11 | 3.25E−08 |
| Skil | 4 | 0.293 | 0.365 | −0.374 | 1.64E−11 | 3.26E−08 |
| Gimap5 | 4 | 0.488 | 0.339 | 0.470 | 4.63E−11 | 8.83E−08 |
| Rsrp1 | 4 | 0.613 | 0.53 | 0.273 | 1.19E−10 | 2.18E−07 |
| Tpm4 | 4 | 0.338 | 0.432 | −0.351 | 1.20E−10 | 2.18E−07 |
| Mir142hg | 4 | 0.1 | 0.164 | −0.659 | 1.28E−10 | 2.32E−07 |
| Sik1 | 4 | 0.221 | 0.322 | −0.485 | 1.49E−10 | 2.67E−07 |
| Ifngr1 | 4 | 0.594 | 0.65 | −0.210 | 3.17E−10 | 5.29E−07 |
| Socs1 | 4 | 0.38 | 0.452 | −0.304 | 3.94E−10 | 6.58E−07 |
| Nfkbie | 4 | 0.242 | 0.325 | −0.368 | 4.10E−10 | 6.82E−07 |
| Dok1 | 4 | 0.336 | 0.214 | 0.446 | 4.37E−10 | 7.21E−07 |
| Tuba1a | 4 | 0.187 | 0.275 | −0.471 | 4.88E−10 | 7.97E−07 |
| Sdhaf1 | 4 | 0.466 | 0.343 | 0.404 | 7.78E−10 | 1.24E−06 |
| Slc15a3 | 4 | 0.199 | 0.275 | −0.484 | 1.00E−09 | 1.58E−06 |
| Egr1 | 4 | 0.483 | 0.557 | −0.338 | 1.14E−09 | 1.78E−06 |
| Slfn2 | 4 | 0.243 | 0.152 | 0.583 | 1.17E−09 | 1.81E−06 |
| Opa3 | 4 | 0.091 | 0.16 | −0.602 | 1.50E−09 | 2.28E−06 |
| Cotl1 | 4 | 0.674 | 0.736 | −0.196 | 1.66E−09 | 2.51E−06 |
| Isy1 | 4 | 0.245 | 0.326 | −0.344 | 2.01E−09 | 3.00E−06 |
| Litaf | 4 | 0.223 | 0.305 | −0.470 | 2.17E−09 | 3.22E−06 |
| Ccdc71l | 4 | 0.154 | 0.257 | −0.629 | 3.10E−09 | 4.51E−06 |
| Ptp4a1 | 4 | 0.268 | 0.305 | −0.253 | 3.57E−09 | 5.14E−06 |
| Birc3 | 4 | 0.361 | 0.426 | −0.276 | 4.40E−09 | 6.28E−06 |
| Cryaa | 4 | 0.104 | 0.162 | −0.447 | 4.59E−09 | 6.53E−06 |
| Lta | 4 | 0.333 | 0.213 | 0.712 | 5.92E−09 | 8.28E−06 |
| Hist1h1c | 4 | 0.285 | 0.182 | 0.597 | 6.38E−09 | 8.85E−06 |
| Dbp | 4 | 0.38 | 0.252 | 0.474 | 6.96E−09 | 9.53E−06 |
| Coq10b | 4 | 0.313 | 0.386 | −0.298 | 7.17E−09 | 9.80E−06 |
| Atf4 | 4 | 0.396 | 0.454 | −0.263 | 7.42E−09 | 1.01E−05 |
| Cytip | 4 | 0.386 | 0.428 | −0.282 | 7.84E−09 | 1.06E−05 |
| Gna13 | 4 | 0.348 | 0.412 | −0.293 | 8.52E−09 | 1.15E−05 |
| Emd | 4 | 0.387 | 0.439 | −0.261 | 9.35E−09 | 1.25E−05 |
| Socs3 | 4 | 0.293 | 0.362 | −0.202 | 9.71E−09 | 1.30E−05 |
| Mdfic | 4 | 0.402 | 0.267 | 0.439 | 1.17E−08 | 1.55E−05 |
| Csrp1 | 4 | 0.158 | 0.21 | −0.549 | 1.30E−08 | 1.71E−05 |
| Mmd | 4 | 0.476 | 0.339 | 0.473 | 1.54E−08 | 1.99E−05 |
| Amica1 | 4 | 0.313 | 0.206 | 0.422 | 1.88E−08 | 2.41E−05 |
| Tsix | 4 | 0.153 | 0.28 | −0.771 | 2.38E−08 | 3.01E−05 |
| Klf2 | 4 | 0.265 | 0.366 | −0.198 | 2.73E−08 | 3.42E−05 |
| D16Ertd472e | 4 | 0.175 | 0.248 | −0.412 | 3.09E−08 | 3.83E−05 |
| Klrk1 | 4 | 0.133 | 0.219 | −0.613 | 6.25E−08 | 7.30E−05 |
| Ttc19 | 4 | 0.152 | 0.23 | −0.499 | 6.38E−08 | 7.43E−05 |
| S100a6 | 4 | 0.603 | 0.517 | 0.370 | 6.87E−08 | 7.98E−05 |
| Rgs3 | 4 | 0.282 | 0.187 | 0.447 | 7.43E−08 | 8.53E−05 |
| Clec2d | 4 | 0.261 | 0.165 | 0.489 | 9.09E−08 | 1.03E−04 |
| Serpinb6b | 4 | 0.2 | 0.26 | −0.432 | 1.10E−07 | 1.22E−04 |
| Neat1 | 4 | 0.502 | 0.378 | 0.447 | 1.25E−07 | 1.38E−04 |
| Ppp2ca | 4 | 0.336 | 0.429 | −0.432 | 1.27E−07 | 1.40E−04 |
| Cdkn1a | 4 | 0.256 | 0.319 | −0.202 | 1.72E−07 | 1.87E−04 |
| Icos | 4 | 0.236 | 0.288 | −0.291 | 2.08E−07 | 2.21E−04 |
| Tubb4b | 4 | 0.347 | 0.396 | −0.252 | 2.30E−07 | 2.42E−04 |
| Nedd8 | 4 | 0.781 | 0.67 | 0.234 | 2.57E−07 | 2.68E−04 |
| Tspan32 | 4 | 0.395 | 0.298 | 0.324 | 2.84E−07 | 2.93E−04 |
| Map3k14 | 4 | 0.133 | 0.202 | −0.515 | 3.33E−07 | 3.39E−04 |
| Dnaja1 | 4 | 0.705 | 0.747 | −0.300 | 3.53E−07 | 3.57E−04 |
| Saraf | 4 | 0.557 | 0.581 | −0.187 | 4.03E−07 | 4.01E−04 |
| Tuba4a | 4 | 0.156 | 0.206 | −0.342 | 4.91E−07 | 4.83E−04 |
| Cd69 | 4 | 0.737 | 0.779 | −0.153 | 5.93E−07 | 5.72E−04 |
| Azin1 | 4 | 0.135 | 0.192 | −0.472 | 6.34E−07 | 6.08 E−04 |
| Minos1 | 4 | 0.768 | 0.696 | 0.218 | 6.36E−07 | 6.08 E−04 |
| Klf13 | 4 | 0.417 | 0.498 | −0.255 | 6.70E−07 | 6.38E−04 |
| Rassf5 | 4 | 0.31 | 0.193 | 0.506 | 7.28E−07 | 6.89E−04 |
| 2310001H17Rik | 4 | 0.214 | 0.133 | 0.484 | 7.78E−07 | 7.30E−04 |
| 4921524J17Rik | 4 | 0.161 | 0.097 | 0.531 | 7.82E−07 | 7.33E−04 |
| Tcrg-C1 | 4 | 0.36 | 0.255 | 0.484 | 9.86E−07 | 9.09E−04 |
| 2010300C02Rik | 4 | 0.518 | 0.411 | 0.310 | 1.02E−06 | 9.35E−04 |
| Alyref | 4 | 0.141 | 0.239 | −0.564 | 1.04E−06 | 9.54E−04 |
| Ramp3 | 4 | 0.224 | 0.289 | −0.380 | 1.14E−06 | 1.04E−03 |
| VgIl4 | 4 | 0.338 | 0.398 | −0.302 | 1.16E−06 | 1.05E−03 |
| Gnb1 | 4 | 0.419 | 0.506 | −0.255 | 1.44E−06 | 1.29E−03 |
| Asb2 | 4 | 0.548 | 0.471 | 0.320 | 1.45E−06 | 1.30E−03 |
| Taf10 | 4 | 0.297 | 0.403 | −0.422 | 1.48E−06 | 1.32E−03 |
| Trps1 | 4 | 0.17 | 0.228 | −0.444 | 1.58E−06 | 1.40E−03 |
| Cacybp | 4 | 0.293 | 0.208 | 0.291 | 2.10E−06 | 1.83E−03 |
| Taf7 | 4 | 0.144 | 0.2 | −0.409 | 2.16E−06 | 1.88E−03 |
| Scn1b | 4 | 0.314 | 0.371 | −0.273 | 2.37E−06 | 2.05E−03 |
| Mast4 | 4 | 0.467 | 0.512 | −0.207 | 2.54E−06 | 2.18E−03 |
| Bin3 | 4 | 0.235 | 0.155 | 0.452 | 2.67E−06 | 2.29E−03 |
| Ppp1r16b | 4 | 0.302 | 0.327 | −0.157 | 2.84E−06 | 2.41E−03 |
| Furin | 4 | 0.382 | 0.433 | −0.203 | 2.91E−06 | 2.46E−03 |
| Rgs1 | 4 | 0.498 | 0.536 | −0.221 | 2.98E−06 | 2.51E−03 |
| Amd1 | 4 | 0.192 | 0.265 | −0.353 | 4.81E−06 | 3.83E−03 |
| Itgav | 4 | 0.175 | 0.227 | −0.394 | 4.91E−06 | 3.90E−03 |
| H2-T23 | 4 | 0.264 | 0.322 | −0.260 | 6.24E−06 | 4.82E−03 |
| S100a10 | 4 | 0.866 | 0.794 | 0.215 | 6.58E−06 | 5.06E−03 |
| Plekhg1 | 4 | 0.231 | 0.295 | −0.229 | 7.07E−06 | 5.39E−03 |
| Ptpn7 | 4 | 0.337 | 0.238 | 0.323 | 7.78E−06 | 5.86E−03 |
| Git2 | 4 | 0.3 | 0.215 | 0.350 | 9.21E−06 | 6.83E−03 |
| Tagap | 4 | 0.461 | 0.366 | 0.423 | 1.02E−05 | 7.46E−03 |
| Morf4l2 | 4 | 0.317 | 0.366 | −0.252 | 1.10E−05 | 8.01E−03 |
| Egln3 | 4 | 0.172 | 0.217 | −0.350 | 1.10E−05 | 8.01E−03 |
| Mbnl2 | 4 | 0.162 | 0.233 | −0.436 | 1.10E−05 | 8.01E−03 |
| Gimap3 | 4 | 0.536 | 0.43 | 0.275 | 1.12E−05 | 8.07E−03 |
| Zfand5 | 4 | 0.252 | 0.306 | −0.245 | 1.12E−05 | 8.09E−03 |
| Ssna1 | 4 | 0.294 | 0.21 | 0.346 | 1.32E−05 | 9.39E−03 |
| Raf1 | 4 | 0.232 | 0.149 | 0.457 | 1.36E−05 | 9.64E−03 |
| Tgoln1 | 4 | 0.286 | 0.324 | −0.209 | 1.42E−05 | 1.00E−02 |
| Trim8 | 4 | 0.223 | 0.138 | 0.530 | 1.48E−05 | 1.04E−02 |
| Rel | 4 | 0.19 | 0.252 | −0.354 | 1.67E−05 | 1.16E−02 |
| Cwc25 | 4 | 0.12 | 0.163 | −0.463 | 1.74E−05 | 1.20E−02 |
| Sat1 | 4 | 0.744 | 0.652 | 0.222 | 1.78E−05 | 1.23E−02 |
| Gatad1 | 4 | 0.155 | 0.2 | −0.316 | 1.89E−05 | 1.29E−02 |
| Fibp | 4 | 0.178 | 0.212 | −0.268 | 2.16E−05 | 1.46E−02 |
| Il2ra | 4 | 0.457 | 0.342 | 0.378 | 2.23E−05 | 1.50E−02 |
| Gcnt2 | 4 | 0.144 | 0.201 | −0.352 | 2.58E−05 | 1.72E−02 |
| Vegfa | 4 | 0.131 | 0.174 | −0.252 | 2.61E−05 | 1.73E−02 |
| Il18r1 | 4 | 0.605 | 0.507 | 0.193 | 2.62E−05 | 1.74E−02 |
| Timm13 | 4 | 0.592 | 0.522 | 0.230 | 3.06E−05 | 2.00E−02 |
| Rcn2 | 4 | 0.258 | 0.302 | −0.225 | 3.10E−05 | 2.02E−02 |
| Irs2 | 4 | 0.149 | 0.201 | −0.372 | 3.24E−05 | 2.10E−02 |
| Fam46a | 4 | 0.099 | 0.152 | −0.500 | 3.45E−05 | 2.22E−02 |
| Leng9 | 4 | 0.128 | 0.179 | −0.375 | 4.41E−05 | 2.77E−02 |
| Herpud1 | 4 | 0.33 | 0.387 | −0.214 | 5.16E−05 | 3.18E−02 |
| Dnajb9 | 4 | 0.18 | 0.236 | −0.322 | 5.37E−05 | 3.29E−02 |
| Zc3hav1 | 4 | 0.145 | 0.184 | −0.303 | 5.44E−05 | 3.33E−02 |
| 1600014C10Rik | 4 | 0.185 | 0.117 | 0.467 | 5.46E−05 | 3.34E−02 |
| Tsc22d3 | 4 | 0.489 | 0.413 | 0.359 | 5.46E−05 | 3.34E−02 |
| Ptger2 | 4 | 0.299 | 0.218 | 0.347 | 5.75E−05 | 3.49E−02 |
| Rpa3 | 4 | 0.175 | 0.101 | 0.541 | 6.42E−05 | 3.82E−02 |
| Tnf | 4 | 0.245 | 0.172 | 0.465 | 6.52E−05 | 3.86E−02 |
| Txk | 4 | 0.529 | 0.59 | −0.188 | 6.82E−05 | 4.02E−02 |
| Sec11a | 4 | 0.376 | 0.297 | 0.283 | 7.54E−05 | 4.40E−02 |
| Serpina3g | 4 | 0.587 | 0.512 | 0.347 | 7.67E−05 | 4.46E−02 |
| Bzw1 | 4 | 0.321 | 0.354 | −0.190 | 8.62E−05 | 4.92E−02 |
| Rnf19b | 4 | 0.427 | 0.454 | −0.242 | 8.70E−05 | 4.94E−02 |
| Irf2 | 4 | 0.306 | 0.216 | 0.312 | 8.71E−05 | 4.94E−02 |
| Tsc22d3 | 5 | 0.84 | 0.565 | 0.772 | 1.41E−53 | 5.84E−49 |
| Bcl2 | 5 | 0.863 | 0.694 | 0.658 | 9.92E−34 | 1.28E−29 |
| Wdr89 | 5 | 0.996 | 0.97 | 0.291 | 1.59E−33 | 1.93E−29 |
| Txnip | 5 | 0.605 | 0.38 | 0.660 | 4.21E−27 | 3.45E−23 |
| Ddit4 | 5 | 0.369 | 0.253 | 0.426 | 2.88E−20 | 1.53E−16 |
| Zfp36l2 | 5 | 0.617 | 0.332 | 0.865 | 1.31E−19 | 6.44E−16 |
| Il7r | 5 | 0.48 | 0.286 | 0.612 | 2.61E−19 | 1.24E−15 |
| Uba52 | 5 | 0.984 | 0.939 | 0.277 | 1.81E−18 | 7.96E−15 |
| Pik3ip1 | 5 | 0.556 | 0.411 | 0.361 | 3.91E−13 | 9.67E−10 |
| Cd74 | 5 | 0.501 | 0.562 | −0.211 | 3.20E−11 | 6.20E−08 |
| DynIl1 | 5 | 0.7 | 0.79 | −0.370 | 4.66E−11 | 8.86E−08 |
| Ifngr1 | 5 | 0.563 | 0.377 | 0.536 | 2.38E−10 | 4.10E−07 |
| Jund | 5 | 0.654 | 0.749 | −0.319 | 1.15E−09 | 1.78E−06 |
| Klhl6 | 5 | 0.332 | 0.208 | 0.485 | 2.58E−09 | 3.78E−06 |
| Pdcd4 | 5 | 0.66 | 0.524 | 0.394 | 2.81E−09 | 4.09E−06 |
| Lfng | 5 | 0.333 | 0.379 | −0.281 | 6.06E−09 | 8.46E−06 |
| Ier2 | 5 | 0.428 | 0.464 | −0.174 | 5.80E−08 | 6.87E−05 |
| Ctsc | 5 | 0.268 | 0.168 | 0.402 | 1.05E−07 | 1.17E−04 |
| Dnaja1 | 5 | 0.591 | 0.71 | −0.416 | 1.91E−07 | 2.05E−04 |
| Ucp2 | 5 | 0.773 | 0.643 | 0.325 | 2.69E−07 | 2.79E−04 |
| Tpm3 | 5 | 0.665 | 0.528 | 0.282 | 3.17E−07 | 3.25E−04 |
| Igha | 5 | 0.197 | 0.127 | 0.503 | 4.85E−07 | 4.77E−04 |
| Tuba1a | 5 | 0.302 | 0.345 | −0.250 | 5.21E−07 | 5.08E−04 |
| Dnajb1 | 5 | 0.206 | 0.351 | −0.867 | 9.86E−07 | 9.09E−04 |
| Dusp1 | 5 | 0.206 | 0.235 | −0.321 | 3.53E−06 | 2.92E−03 |
| Slc9a3r1 | 5 | 0.428 | 0.507 | −0.256 | 5.20E−06 | 4.11E−03 |
| Hspa8 | 5 | 0.977 | 0.975 | −0.255 | 6.72E−06 | 5.15E−03 |
| Sfpq | 5 | 0.284 | 0.366 | −0.373 | 6.74E−06 | 5.15E−03 |
| Tdrp | 5 | 0.228 | 0.277 | −0.354 | 9.01E−06 | 6.69E−03 |
| Ppp2ca | 5 | 0.277 | 0.376 | −0.469 | 1.12E−05 | 8.10E−03 |
| Gramd3 | 5 | 0.475 | 0.302 | 0.481 | 1.65E−05 | 1.14E−02 |
| Calm1 | 5 | 0.868 | 0.891 | −0.192 | 1.69E−05 | 1.17E−02 |
| Ppp3cc | 5 | 0.152 | 0.074 | 0.621 | 1.87E−05 | 1.29E−02 |
| Klf6 | 5 | 0.368 | 0.437 | −0.374 | 2.48E−05 | 1.66E−02 |
| 1810037l17Rik | 5 | 0.446 | 0.473 | −0.158 | 2.48E−05 | 1.66E−02 |
| Crlf3 | 5 | 0.569 | 0.421 | 0.370 | 3.69E−05 | 2.36E−02 |
| Rnaset2a | 5 | 0.709 | 0.583 | 0.225 | 4.18E−05 | 2.64E−02 |
| Ppp1r15a | 5 | 0.219 | 0.257 | −0.287 | 4.73E−05 | 2.95E−02 |
| Ltb | 5 | 0.865 | 0.893 | −0.238 | 5.43E−05 | 3.33E−02 |
| Ilk | 5 | 0.2 | 0.15 | 0.159 | 7.23E−05 | 4.22E−02 |
| Racgap1 | 5 | 0.21 | 0.135 | 0.436 | 8.16E−05 | 4.70E−02 |
| Mcpt1 | 6 | 0.35 | 0.058 | 0.479 | 8.41E−63 | 5.09E−58 |
| Defa24 | 6 | 0.099 | 0.298 | −1.145 | 2.72E−44 | 6.70E−40 |
| Jund | 6 | 0.92 | 0.964 | −0.422 | 2.90E−43 | 6.92E−39 |
| Igha | 6 | 0.681 | 0.424 | 0.596 | 4.93E−39 | 7.92E−35 |
| Mcpt2 | 6 | 0.212 | 0.03 | 0.749 | 2.04E−38 | 3.16E−34 |
| Lyz1 | 6 | 0.035 | 0.174 | −1.858 | 2.01E−37 | 2.99E−33 |
| Ppia | 6 | 0.998 | 0.998 | 0.186 | 9.41E−36 | 1.30E−31 |
| Ddit4 | 6 | 0.221 | 0.077 | 1.322 | 7.53E−31 | 8.01E−27 |
| Junb | 6 | 0.745 | 0.856 | −0.174 | 1.91E−30 | 2.01E−26 |
| Fabp2 | 6 | 0.048 | 0.187 | −1.495 | 4.37E−28 | 4.00E−24 |
| Psap | 6 | 0.997 | 0.999 | 0.202 | 6.20E−28 | 5.55E−24 |
| Nr4a1 | 6 | 0.508 | 0.62 | −0.422 | 8.34E−28 | 7.30E−24 |
| Egr1 | 6 | 0.143 | 0.243 | −0.987 | 2.00E−27 | 1.72E−23 |
| Fosb | 6 | 0.332 | 0.475 | −0.556 | 3.19E−25 | 2.28E−21 |
| Klf4 | 6 | 0.147 | 0.286 | −0.763 | 1.96E−23 | 1.28E−19 |
| Jun | 6 | 0.769 | 0.66 | 0.499 | 9.00E−20 | 4.54E−16 |
| Ptpre | 6 | 0.392 | 0.534 | −0.452 | 2.12E−19 | 1.02E−15 |
| Neat1 | 6 | 0.885 | 0.81 | 0.364 | 1.57E−17 | 6.12E−14 |
| Hist1h1c | 6 | 0.522 | 0.364 | 0.792 | 2.52E−17 | 9.66E−14 |
| Mir142hg | 6 | 0.169 | 0.289 | −0.649 | 6.19E−17 | 2.29E−13 |
| Pnrc1 | 6 | 0.706 | 0.761 | −0.212 | 2.17E−16 | 7.64E−13 |
| Grasp | 6 | 0.193 | 0.309 | −0.617 | 2.21E−16 | 7.72E−13 |
| Rilpl2 | 6 | 0.851 | 0.863 | −0.158 | 3.71E−16 | 1.27E−12 |
| Dusp5 | 6 | 0.668 | 0.711 | −0.167 | 6.13E−16 | 2.02E−12 |
| Kdm6b | 6 | 0.307 | 0.397 | −0.488 | 1.04E−15 | 3.34E−12 |
| Bcl2l11 | 6 | 0.257 | 0.35 | −0.499 | 2.99E−15 | 9.09E−12 |
| Smc6 | 6 | 0.849 | 0.841 | −0.180 | 1.25E−14 | 3.59E−11 |
| Bhlhe40 | 6 | 0.465 | 0.543 | −0.297 | 1.25E−14 | 3.59E−11 |
| Ier2 | 6 | 0.432 | 0.538 | −0.265 | 2.19E−14 | 6.02E−11 |
| Dnajc7 | 6 | 0.987 | 0.976 | 0.197 | 6.20E−14 | 1.65E−10 |
| Ier5 | 6 | 0.937 | 0.9 | 0.332 | 2.06E−13 | 5.27E−10 |
| Hist2h4 | 6 | 0.165 | 0.066 | 1.036 | 3.04E−13 | 7.66E−10 |
| Mpeg1 | 6 | 0.992 | 0.975 | 0.185 | 3.52E−13 | 8.76E−10 |
| VgIl4 | 6 | 0.34 | 0.455 | −0.419 | 4.36E−13 | 1.06E−09 |
| Ifrd1 | 6 | 0.241 | 0.319 | −0.459 | 4.97E−13 | 1.21E−09 |
| Igkc | 6 | 0.944 | 0.856 | 0.194 | 5.84E−13 | 1.40E−09 |
| Ppp2ca | 6 | 0.539 | 0.637 | −0.356 | 6.35E−13 | 1.51E−09 |
| Napsa | 6 | 0.888 | 0.809 | 0.245 | 7.31E−13 | 1.73E−09 |
| Klk1 | 6 | 0.964 | 0.956 | 0.243 | 8.31E−13 | 1.96E−09 |
| Dusp18 | 6 | 0.091 | 0.161 | −0.789 | 1.03E−12 | 2.35E−09 |
| Rnase6 | 6 | 0.991 | 0.994 | 0.160 | 1.39E−12 | 3.13E−09 |
| Filip1l | 6 | 0.384 | 0.471 | −0.295 | 2.43E−12 | 5.31E−09 |
| Spsb2 | 6 | 0.187 | 0.275 | −0.508 | 3.32E−12 | 7.11E−09 |
| Vps37b | 6 | 0.424 | 0.489 | −0.292 | 6.60E−12 | 1.38E−08 |
| Ccl4 | 6 | 0.848 | 0.889 | 0.241 | 7.42E−12 | 1.52E−08 |
| Odc1 | 6 | 0.366 | 0.44 | −0.325 | 9.58E−12 | 1.95E−08 |
| Clec2d | 6 | 0.578 | 0.438 | 0.409 | 1.12E−11 | 2.28E−08 |
| H2-Ab1 | 6 | 0.949 | 0.947 | 0.238 | 1.36E−11 | 2.75E−08 |
| Cxcr4 | 6 | 0.561 | 0.407 | 0.491 | 1.64E−11 | 3.26E−08 |
| Pltp | 6 | 0.878 | 0.806 | 0.260 | 1.78E−11 | 3.52E−08 |
| D13Ertd608e | 6 | 0.947 | 0.901 | 0.276 | 3.28E−11 | 6.34E−08 |
| Dusp1 | 6 | 0.406 | 0.494 | −0.366 | 3.41E−11 | 6.58E−08 |
| Nr4a3 | 6 | 0.134 | 0.197 | −0.556 | 4.12E−11 | 7.90E−08 |
| Plek | 6 | 0.617 | 0.652 | −0.205 | 4.40E−11 | 8.40E−08 |
| Fyn | 6 | 0.756 | 0.799 | −0.230 | 9.87E−11 | 1.83E−07 |
| Rel | 6 | 0.381 | 0.454 | −0.305 | 1.01E−10 | 1.87E−07 |
| Fam46c | 6 | 0.221 | 0.306 | −0.440 | 1.30E−10 | 2.35E−07 |
| Wnk1 | 6 | 0.605 | 0.645 | −0.262 | 1.60E−10 | 2.85E−07 |
| Rpn2 | 6 | 0.664 | 0.569 | 0.272 | 1.79E−10 | 3.18E−07 |
| H2-Aa | 6 | 0.947 | 0.949 | 0.220 | 1.83E−10 | 3.24E−07 |
| Pim1 | 6 | 0.509 | 0.58 | −0.188 | 2.16E−10 | 3.77E−07 |
| Rgs1 | 6 | 0.466 | 0.351 | 0.548 | 5.04E−10 | 8.21E−07 |
| Pik3ip1 | 6 | 0.423 | 0.307 | 0.445 | 5.80E−10 | 9.38E−07 |
| Runx3 | 6 | 0.53 | 0.603 | −0.200 | 1.09E−09 | 1.70E−06 |
| B3gnt8 | 6 | 0.486 | 0.362 | 0.265 | 1.17E−09 | 1.81E−06 |
| Ppp1r16b | 6 | 0.237 | 0.315 | −0.419 | 1.41E−09 | 2.15E−06 |
| Selplg | 6 | 0.992 | 0.973 | 0.183 | 1.76E−09 | 2.67E−06 |
| Atf3 | 6 | 0.091 | 0.161 | −0.693 | 1.95E−09 | 2.92E−06 |
| Npc2 | 6 | 0.985 | 0.975 | 0.166 | 2.14E−09 | 3.18E−06 |
| Skil | 6 | 0.435 | 0.477 | −0.246 | 2.74E−09 | 4.01E−06 |
| Traf4 | 6 | 0.535 | 0.592 | −0.199 | 2.88E−09 | 4.19E−06 |
| Emp3 | 6 | 0.936 | 0.856 | 0.172 | 4.65E−09 | 6.61E−06 |
| Foxp1 | 6 | 0.719 | 0.741 | −0.169 | 4.72E−09 | 6.68E−06 |
| Ly6c1 | 6 | 0.991 | 0.99 | −0.173 | 5.07E−09 | 7.14E−06 |
| Zfp36l2 | 6 | 0.783 | 0.66 | 0.365 | 7.84E−09 | 1.06E−05 |
| Ifi30 | 6 | 0.883 | 0.807 | 0.256 | 1.09E−08 | 1.45E−05 |
| Zc3h12a | 6 | 0.135 | 0.198 | −0.495 | 1.43E−08 | 1.86E−05 |
| Lag3 | 6 | 0.605 | 0.489 | 0.315 | 2.39E−08 | 3.01E−05 |
| Plekho2 | 6 | 0.201 | 0.271 | −0.406 | 2.76E−08 | 3.44E−05 |
| S1pr4 | 6 | 0.351 | 0.27 | 0.331 | 3.95E−08 | 4.83E−05 |
| Trim25 | 6 | 0.339 | 0.394 | −0.239 | 4.20E−08 | 5.10E−05 |
| Lgals1 | 6 | 0.985 | 0.97 | 0.174 | 4.90E−08 | 5.89E−05 |
| Ptp4a1 | 6 | 0.4 | 0.412 | −0.201 | 5.43E−08 | 6.48E−05 |
| Erdr1 | 6 | 0.149 | 0.2 | −0.520 | 5.84E−08 | 6.91E−05 |
| Tgif1 | 6 | 0.115 | 0.174 | −0.548 | 6.25E−08 | 7.30E−05 |
| H2-Eb1 | 6 | 0.907 | 0.891 | 0.228 | 8.18E−08 | 9.36E−05 |
| Clec10a | 6 | 0.532 | 0.426 | 0.327 | 8.43E−08 | 9.63E−05 |
| Gapdh | 6 | 0.911 | 0.848 | 0.206 | 9.39E−08 | 1.06E−04 |
| Gtf2i | 6 | 0.542 | 0.484 | 0.283 | 9.52E−08 | 1.08E−04 |
| Atp5h | 6 | 0.963 | 0.92 | 0.168 | 9.81E−08 | 1.10E−04 |
| Cd68 | 6 | 0.861 | 0.798 | 0.204 | 1.25E−07 | 1.38E−04 |
| Itpkb | 6 | 0.296 | 0.363 | −0.230 | 1.78E−07 | 1.93E−04 |
| Rnf19b | 6 | 0.255 | 0.303 | −0.314 | 1.88E−07 | 2.02E−04 |
| Kmt2e | 6 | 0.596 | 0.634 | −0.156 | 1.91E−07 | 2.05E−04 |
| Ddx3x | 6 | 0.436 | 0.489 | −0.202 | 2.38E−07 | 2.50E−04 |
| Crem | 6 | 0.34 | 0.38 | −0.279 | 2.77E−07 | 2.87E−04 |
| Zfand5 | 6 | 0.423 | 0.496 | −0.220 | 3.13E−07 | 3.21E−04 |
| Sell | 6 | 0.983 | 0.962 | 0.172 | 3.31E−07 | 3.38E−04 |
| Nfkbiz | 6 | 0.336 | 0.381 | −0.288 | 3.37E−07 | 3.42E−04 |
| Coq10b | 6 | 0.242 | 0.311 | −0.336 | 3.65E−07 | 3.66E−04 |
| Cd83 | 6 | 0.092 | 0.155 | −0.472 | 3.80E−07 | 3.80E−04 |
| Lmnb1 | 6 | 0.189 | 0.257 | −0.415 | 4.02E−07 | 4.01E−04 |
| Sla2 | 6 | 0.735 | 0.605 | 0.243 | 4.24E−07 | 4.23E−04 |
| Cxcr3 | 6 | 0.516 | 0.405 | 0.302 | 4.51E−07 | 4.47E−04 |
| Hes6 | 6 | 0.474 | 0.367 | 0.290 | 5.14E−07 | 5.04E−04 |
| Sf3b1 | 6 | 0.706 | 0.627 | 0.237 | 5.35E−07 | 5.21E−04 |
| Gnb1 | 6 | 0.515 | 0.574 | −0.247 | 5.60E−07 | 5.41E−04 |
| AY036118 | 6 | 0.426 | 0.414 | −0.256 | 6.27E−07 | 6.02E−04 |
| Siah2 | 6 | 0.253 | 0.318 | −0.303 | 7.71E−07 | 7.24E−04 |
| Fosl2 | 6 | 0.141 | 0.176 | −0.387 | 9.82E−07 | 9.08E−04 |
| Gapt | 6 | 0.657 | 0.723 | −0.204 | 1.23E−06 | 1.11E−03 |
| Fkbp5 | 6 | 0.161 | 0.093 | 0.573 | 1.31E−06 | 1.18E−03 |
| Cdv3 | 6 | 0.414 | 0.479 | −0.219 | 1.33E−06 | 1.20E−03 |
| Tnfaip8l2 | 6 | 0.512 | 0.419 | 0.307 | 1.42E−06 | 1.27E−03 |
| Btg2 | 6 | 0.801 | 0.841 | −0.156 | 1.51E−06 | 1.34E−03 |
| Mcl1 | 6 | 0.459 | 0.495 | −0.199 | 1.94E−06 | 1.71E−03 |
| Socs3 | 6 | 0.55 | 0.49 | 0.385 | 2.68E−06 | 2.29E−03 |
| Bloc1s2 | 6 | 0.784 | 0.694 | 0.218 | 2.78E−06 | 2.37E−03 |
| Nfkbia | 6 | 0.661 | 0.695 | −0.191 | 2.85E−06 | 2.41E−03 |
| Os9 | 6 | 0.441 | 0.365 | 0.265 | 2.87E−06 | 2.43E−03 |
| Clcn3 | 6 | 0.35 | 0.383 | −0.239 | 3.30E−06 | 2.76E−03 |
| H2afj | 6 | 0.935 | 0.878 | 0.166 | 3.66E−06 | 3.01E−03 |
| Pink1 | 6 | 0.282 | 0.337 | −0.291 | 4.12E−06 | 3.34E−03 |
| Sema4b | 6 | 0.531 | 0.453 | 0.221 | 4.49E−06 | 3.62E−03 |
| Ablim1 | 6 | 0.403 | 0.451 | −0.197 | 4.87E−06 | 3.87E−03 |
| Rsrp1 | 6 | 0.754 | 0.701 | 0.195 | 5.54E−06 | 4.36E−03 |
| Map3k1 | 6 | 0.346 | 0.397 | −0.227 | 5.60E−06 | 4.39E−03 |
| Ctsz | 6 | 0.97 | 0.939 | 0.156 | 5.69E−06 | 4.45E−03 |
| Gadd45b | 6 | 0.342 | 0.413 | −0.220 | 6.21E−06 | 4.80E−03 |
| H2-DMa | 6 | 0.878 | 0.801 | 0.190 | 6.66E−06 | 5.12E−03 |
| Gnai2 | 6 | 0.932 | 0.939 | −0.175 | 6.87E−06 | 5.24E−03 |
| Irf4 | 6 | 0.256 | 0.304 | −0.248 | 9.39E−06 | 6.95E−03 |
| Ptprcap | 6 | 0.941 | 0.894 | 0.159 | 1.09E−05 | 7.99E−03 |
| Pten | 6 | 0.429 | 0.475 | −0.226 | 1.11E−05 | 8.01E−03 |
| Tmem86a | 6 | 0.204 | 0.151 | 0.323 | 1.21E−05 | 8.69E−03 |
| Grb2 | 6 | 0.614 | 0.678 | −0.222 | 1.23E−05 | 8.79E−03 |
| Orai1 | 6 | 0.274 | 0.339 | −0.245 | 1.48E−05 | 1.04E−02 |
| Itgb7 | 6 | 0.669 | 0.574 | 0.238 | 1.65E−05 | 1.14E−02 |
| Paqr5 | 6 | 0.52 | 0.453 | 0.279 | 1.81E−05 | 1.25E−02 |
| Snrnp70 | 6 | 0.43 | 0.347 | 0.220 | 1.88E−05 | 1.29E−02 |
| Sertad1 | 6 | 0.12 | 0.173 | −0.396 | 1.94E−05 | 1.32E−02 |
| Klf3 | 6 | 0.125 | 0.177 | −0.428 | 2.01E−05 | 1.36E−02 |
| Hnrnpa3 | 6 | 0.851 | 0.799 | 0.164 | 2.53E−05 | 1.69E−02 |
| Tsix | 6 | 0.23 | 0.271 | −0.418 | 2.64E−05 | 1.75E−02 |
| Cdkn2d | 6 | 0.747 | 0.658 | 0.244 | 2.68E−05 | 1.77E−02 |
| Rab7b | 6 | 0.223 | 0.257 | −0.285 | 3.09E−05 | 2.02E−02 |
| Fcer1g | 6 | 0.94 | 0.895 | 0.160 | 3.14E−05 | 2.05E−02 |
| Tmem59 | 6 | 0.833 | 0.779 | 0.179 | 3.23E−05 | 2.10E−02 |
| Klk1b27 | 6 | 0.974 | 0.955 | 0.170 | 3.69E−05 | 2.36E−02 |
| Tmem50a | 6 | 0.854 | 0.801 | 0.153 | 3.90E−05 | 2.48E−02 |
| Txndc5 | 6 | 0.699 | 0.623 | 0.226 | 4.00E−05 | 2.54E−02 |
| Lifr | 6 | 0.595 | 0.489 | 0.265 | 4.03E−05 | 2.55E−02 |
| Arid3a | 6 | 0.212 | 0.247 | −0.329 | 4.35E−05 | 2.74E−02 |
| Pde4b | 6 | 0.177 | 0.227 | −0.282 | 4.84E−05 | 3.01E−02 |
| Mark3 | 6 | 0.139 | 0.184 | −0.383 | 4.92E−05 | 3.05E−02 |
| Pdia3 | 6 | 0.844 | 0.792 | 0.157 | 5.00E−05 | 3.09E−02 |
| Gatad1 | 6 | 0.34 | 0.391 | −0.199 | 5.83E−05 | 3.53E−02 |
| Arl4a | 6 | 0.228 | 0.289 | −0.276 | 6.07E−05 | 3.64E−02 |
| Asph | 6 | 0.377 | 0.405 | −0.195 | 7.17E−05 | 4.20E−02 |
| Net1 | 6 | 0.53 | 0.44 | 0.285 | 7.94E−05 | 4.59E−02 |
| Cfp | 6 | 0.422 | 0.358 | 0.312 | 8.22E−05 | 4.72E−02 |
| 5730508B09Rik | 6 | 0.268 | 0.313 | −0.218 | 8.68E−05 | 4.94E−02 |
| Zfp36l2 | 7 | 0.757 | 0.354 | 1.239 | 4.40E−35 | 5.97E−31 |
| Tsc22d3 | 7 | 0.565 | 0.294 | 1.003 | 1.83E−19 | 8.83E−16 |
| Ddit4 | 7 | 0.359 | 0.273 | 0.463 | 2.18E−14 | 5.99E−11 |
| Lmo4 | 7 | 0.204 | 0.135 | 0.711 | 1.35E−10 | 2.43E−07 |
| Dusp2 | 7 | 0.465 | 0.327 | 0.542 | 4.00E−09 | 5.74E−06 |
| Txnip | 7 | 0.415 | 0.275 | 0.551 | 7.43E−09 | 1.01E−05 |
| Ptpn22 | 7 | 0.53 | 0.429 | 0.340 | 3.19E−06 | 2.67E−03 |
| Fkbp5 | 7 | 0.167 | 0.091 | 0.540 | 4.79E−06 | 3.82E−03 |
| Il7r | 7 | 0.191 | 0.123 | 0.526 | 7.22E−06 | 5.48E−03 |
| Hsp90ab1 | 7 | 0.834 | 0.891 | −0.450 | 7.82E−06 | 5.88E−03 |
| Matk | 7 | 0.284 | 0.173 | 0.513 | 3.61E−05 | 2.32E−02 |
| Gabarapl2 | 7 | 0.643 | 0.554 | 0.253 | 6.23E−05 | 3.72E−02 |
| Svbp | 7 | 0.246 | 0.178 | 0.261 | 7.76E−05 | 4.50E−02 |
| Jund | 8 | 0.846 | 0.905 | −0.703 | 1.54E−45 | 4.48E−41 |
| Txnip | 8 | 0.398 | 0.146 | 1.190 | 8.77E−36 | 1.23E−31 |
| Kdm6b | 8 | 0.274 | 0.451 | −0.833 | 9.22E−28 | 7.97E−24 |
| Nfkbia | 8 | 0.614 | 0.778 | −0.579 | 6.04E−27 | 4.90E−23 |
| Crem | 8 | 0.371 | 0.502 | −0.681 | 1.50E−23 | 9.85E−20 |
| Mcpt1 | 8 | 0.209 | 0.037 | 1.756 | 5.20E−23 | 3.28E−19 |
| Nfkbid | 8 | 0.146 | 0.323 | −1.010 | 7.52E−23 | 4.70E−19 |
| Ifrd1 | 8 | 0.276 | 0.434 | −0.708 | 7.62E−22 | 4.41E−18 |
| Defa24 | 8 | 0.044 | 0.172 | −0.967 | 8.62E−22 | 4.95E−18 |
| Nr4a1 | 8 | 0.561 | 0.698 | −0.415 | 3.63E−21 | 2.02E−17 |
| Nfkbiz | 8 | 0.358 | 0.471 | −0.640 | 1.02E−19 | 5.07E−16 |
| Litaf | 8 | 0.339 | 0.488 | −0.518 | 1.52E−18 | 6.74E−15 |
| Rgs1 | 8 | 0.699 | 0.507 | 0.795 | 2.13E−18 | 9.31E−15 |
| Nr4a3 | 8 | 0.157 | 0.272 | −0.808 | 5.15E−18 | 2.18E−14 |
| Gadd45b | 8 | 0.17 | 0.326 | −0.563 | 8.88E−18 | 3.60E−14 |
| Ier5l | 8 | 0.057 | 0.16 | −1.422 | 4.06E−17 | 1.54E−13 |
| H3f3b | 8 | 0.993 | 0.996 | −0.185 | 5.58E−17 | 2.08E−13 |
| AW112010 | 8 | 0.995 | 0.996 | 0.208 | 2.63E−16 | 9.12E−13 |
| Id2 | 8 | 0.684 | 0.497 | 0.467 | 2.67E−16 | 9.22E−13 |
| Ncr1 | 8 | 0.964 | 0.931 | 0.265 | 4.30E−16 | 1.45E−12 |
| Lmna | 8 | 0.089 | 0.207 | −1.022 | 1.00E−15 | 3.23E−12 |
| Nr4a2 | 8 | 0.348 | 0.486 | −0.530 | 1.28E−15 | 4.07E−12 |
| Prr7 | 8 | 0.093 | 0.208 | −0.910 | 2.04E−15 | 6.31E−12 |
| VgIl4 | 8 | 0.47 | 0.602 | −0.452 | 2.25E−15 | 6.92E−12 |
| Tsc22d3 | 8 | 0.664 | 0.475 | 0.472 | 3.58E−15 | 1.07E−11 |
| Tgif1 | 8 | 0.305 | 0.437 | −0.580 | 4.29E−15 | 1.28E−11 |
| Cd74 | 8 | 0.448 | 0.563 | −0.187 | 5.48E−15 | 1.60E−11 |
| Hist1h1e | 8 | 0.176 | 0.047 | 1.277 | 1.14E−14 | 3.28E−11 |
| Nkg7 | 8 | 0.985 | 0.981 | 0.194 | 6.12E−14 | 1.63E−10 |
| Gzma | 8 | 0.764 | 0.719 | 0.340 | 9.80E−14 | 2.57E−10 |
| AY036118 | 8 | 0.431 | 0.444 | −0.339 | 1.27E−13 | 3.31E−10 |
| Mir142hg | 8 | 0.161 | 0.29 | −0.715 | 2.41E−13 | 6.14E−10 |
| Zfp36l2 | 8 | 0.84 | 0.66 | 0.382 | 4.30E−13 | 1.05E−09 |
| Traf1 | 8 | 0.464 | 0.552 | −0.355 | 4.30E−13 | 1.05E−09 |
| Cxcr4 | 8 | 0.577 | 0.349 | 0.544 | 5.87E−13 | 1.40E−09 |
| Egr1 | 8 | 0.156 | 0.256 | −0.640 | 5.89E−13 | 1.40E−09 |
| Zc3h12a | 8 | 0.182 | 0.288 | −0.680 | 1.19E−12 | 2.71E−09 |
| Klrc1 | 8 | 0.608 | 0.463 | 0.392 | 2.12E−12 | 4.69E−09 |
| Klrb1f | 8 | 0.56 | 0.394 | 0.487 | 2.48E−12 | 5.40E−09 |
| Cdkn1a | 8 | 0.084 | 0.191 | −0.980 | 2.74E−12 | 5.93E−09 |
| Ftl1 | 8 | 0.963 | 0.977 | −0.171 | 4.25E−12 | 9.04E−09 |
| Fosl2 | 8 | 0.378 | 0.418 | −0.358 | 6.97E−12 | 1.44E−08 |
| Icam1 | 8 | 0.175 | 0.304 | −0.819 | 7.07E−12 | 1.46E−08 |
| Pnrc1 | 8 | 0.769 | 0.836 | −0.302 | 8.05E−12 | 1.65E−08 |
| Odc1 | 8 | 0.202 | 0.284 | −0.540 | 1.51E−11 | 3.04E−08 |
| Pim1 | 8 | 0.677 | 0.77 | −0.358 | 2.02E−11 | 3.95E−08 |
| Igha | 8 | 0.488 | 0.302 | 0.514 | 6.38E−11 | 1.21E−07 |
| Emb | 8 | 0.506 | 0.54 | −0.223 | 9.10E−11 | 1.69E−07 |
| Zfp512 | 8 | 0.446 | 0.273 | 0.523 | 1.36E−10 | 2.44E−07 |
| Hist1h1c | 8 | 0.348 | 0.217 | 0.586 | 2.05E−10 | 3.61E−07 |
| Ptprcap | 8 | 0.929 | 0.844 | 0.276 | 2.51E−10 | 4.30E−07 |
| Vps37b | 8 | 0.79 | 0.825 | −0.178 | 2.53E−10 | 4.33E−07 |
| Rel | 8 | 0.139 | 0.222 | −0.595 | 2.67E−10 | 4.54E−07 |
| Gramd3 | 8 | 0.227 | 0.338 | −0.502 | 3.02E−10 | 5.06E−07 |
| Klre1 | 8 | 0.744 | 0.645 | 0.295 | 4.38E−10 | 7.22E−07 |
| Ms4a6b | 8 | 0.419 | 0.257 | 0.490 | 5.22E−10 | 8.47E−07 |
| Igkc | 8 | 0.677 | 0.522 | 0.390 | 8.49E−10 | 1.35E−06 |
| Klrd1 | 8 | 0.939 | 0.866 | 0.261 | 1.17E−09 | 1.81E−06 |
| H2-K1 | 8 | 0.978 | 0.991 | 0.192 | 1.37E−09 | 2.10E−06 |
| Ikzf2 | 8 | 0.216 | 0.283 | −0.467 | 1.82E−09 | 2.74E−06 |
| Ptp4a2 | 8 | 0.606 | 0.7 | −0.243 | 2.08E−09 | 3.11E−06 |
| Ms4a4b | 8 | 0.953 | 0.903 | 0.271 | 2.28E−09 | 3.39E−06 |
| Rrad | 8 | 0.191 | 0.264 | −0.602 | 3.81E−09 | 5.49E−06 |
| Maff | 8 | 0.251 | 0.326 | −0.445 | 6.29E−09 | 8.76E−06 |
| Coq10b | 8 | 0.305 | 0.401 | −0.426 | 1.31E−08 | 1.72E−05 |
| Tfam | 8 | 0.19 | 0.248 | −0.555 | 1.44E−08 | 1.87E−05 |
| Phlda1 | 8 | 0.134 | 0.234 | −0.867 | 1.86E−08 | 2.38E−05 |
| Klrc2 | 8 | 0.505 | 0.376 | 0.396 | 1.92E−08 | 2.45E−05 |
| B2m | 8 | 0.999 | 0.997 | 0.159 | 2.10E−08 | 2.66E−05 |
| Fcer1g | 8 | 0.927 | 0.913 | 0.170 | 3.13E−08 | 3.88E−05 |
| Arl6ip5 | 8 | 0.618 | 0.461 | 0.344 | 3.31E−08 | 4.08E−05 |
| Gimap9 | 8 | 0.511 | 0.364 | 0.367 | 3.43E−08 | 4.23E−05 |
| Ddx21 | 8 | 0.248 | 0.322 | −0.379 | 4.05E−08 | 4.94E−05 |
| Ifi203 | 8 | 0.547 | 0.387 | 0.402 | 4.11E−08 | 5.01E−05 |
| Arl6ip1 | 8 | 0.61 | 0.447 | 0.367 | 4.47E−08 | 5.40E−05 |
| Eif4a2 | 8 | 0.622 | 0.453 | 0.300 | 6.14E−08 | 7.21E−05 |
| Myl6 | 8 | 0.987 | 0.955 | 0.199 | 7.12E−08 | 8.23E−05 |
| Ier2 | 8 | 0.781 | 0.851 | −0.235 | 9.00E−08 | 1.02E−04 |
| Eef2 | 8 | 0.957 | 0.896 | 0.257 | 1.06E−07 | 1.19E−04 |
| Txndc5 | 8 | 0.239 | 0.137 | 0.592 | 1.21E−07 | 1.34E−04 |
| Gimap1 | 8 | 0.776 | 0.641 | 0.283 | 1.45E−07 | 1.59E−04 |
| Klrb1c | 8 | 0.735 | 0.621 | 0.233 | 1.78E−07 | 1.93E−04 |
| Actr3 | 8 | 0.771 | 0.639 | 0.315 | 2.06E−07 | 2.20E−04 |
| Gnai2 | 8 | 0.661 | 0.685 | −0.261 | 2.16E−07 | 2.30E−04 |
| Ppp1r16b | 8 | 0.206 | 0.28 | −0.367 | 2.26E−07 | 2.39E−04 |
| Fasl | 8 | 0.598 | 0.488 | 0.296 | 2.30E−07 | 2.42E−04 |
| Ube2s | 8 | 0.478 | 0.535 | −0.248 | 2.30E−07 | 2.42E−04 |
| Ppp1r15a | 8 | 0.406 | 0.499 | −0.355 | 2.52E−07 | 2.63E−04 |
| Gimap6 | 8 | 0.815 | 0.682 | 0.272 | 3.06E−07 | 3.15E−04 |
| Amd1 | 8 | 0.265 | 0.34 | −0.426 | 3.32E−07 | 3.38E−04 |
| Dusp1 | 8 | 0.614 | 0.702 | −0.244 | 4.56E−07 | 4.51E−04 |
| Sh2d1a | 8 | 0.488 | 0.367 | 0.388 | 6.06E−07 | 5.83E−04 |
| Nfkb1 | 8 | 0.374 | 0.425 | −0.244 | 6.15E−07 | 5.91E−04 |
| Fam105a | 8 | 0.376 | 0.26 | 0.376 | 7.53E−07 | 7.10E−04 |
| Bcl2 | 8 | 0.839 | 0.743 | 0.286 | 7.94E−07 | 7.43E−04 |
| Alyref | 8 | 0.137 | 0.207 | −0.597 | 8.08E−07 | 7.55E−04 |
| Arfrp1 | 8 | 0.21 | 0.119 | 0.609 | 8.64E−07 | 8.02E−04 |
| Taf10 | 8 | 0.347 | 0.414 | −0.372 | 9.27E−07 | 8.59E−04 |
| Sik1 | 8 | 0.161 | 0.229 | −0.527 | 1.00E−06 | 9.23E−04 |
| Cdk2ap1 | 8 | 0.132 | 0.215 | −0.673 | 1.04E−06 | 9.53E−04 |
| Rac2 | 8 | 0.97 | 0.949 | 0.169 | 1.22E−06 | 1.11E−03 |
| Ctla2a | 8 | 0.74 | 0.637 | 0.372 | 1.39E−06 | 1.25E−03 |
| Skil | 8 | 0.207 | 0.271 | −0.410 | 1.77E−06 | 1.56E−03 |
| Ccr2 | 8 | 0.536 | 0.39 | 0.330 | 2.01E−06 | 1.76E−03 |
| Sla | 8 | 0.335 | 0.218 | 0.486 | 2.10E−06 | 1.83E−03 |
| Cd47 | 8 | 0.833 | 0.739 | 0.262 | 2.56E−06 | 2.20E−03 |
| Dgat1 | 8 | 0.175 | 0.229 | −0.352 | 2.67E−06 | 2.29E−03 |
| Tm6sf1 | 8 | 0.454 | 0.334 | 0.360 | 2.71E−06 | 2.31E−03 |
| Gramd1a | 8 | 0.336 | 0.214 | 0.460 | 3.06E−06 | 2.57E−03 |
| Il2rg | 8 | 0.681 | 0.727 | −0.226 | 3.62E−06 | 2.98E−03 |
| Sp100 | 8 | 0.537 | 0.411 | 0.330 | 3.65E−06 | 3.00E−03 |
| Ninj1 | 8 | 0.125 | 0.168 | −0.512 | 3.92E−06 | 3.19E−03 |
| Klrk1 | 8 | 0.873 | 0.763 | 0.213 | 4.14E−06 | 3.36E−03 |
| Rnf125 | 8 | 0.167 | 0.241 | −0.483 | 4.94E−06 | 3.91E−03 |
| Bcl2l11 | 8 | 0.383 | 0.425 | −0.208 | 5.91E−06 | 4.60E−03 |
| Neurl3 | 8 | 0.388 | 0.429 | −0.244 | 6.01E−06 | 4.67E−03 |
| Rora | 8 | 0.161 | 0.227 | −0.455 | 6.55E−06 | 5.05E−03 |
| Nabp1 | 8 | 0.702 | 0.683 | −0.167 | 8.55E−06 | 6.38E−03 |
| Csrp1 | 8 | 0.129 | 0.179 | −0.437 | 1.12E−05 | 8.10E−03 |
| Cks2 | 8 | 0.108 | 0.156 | −0.601 | 1.12E−05 | 8.10E−03 |
| Rnf157 | 8 | 0.188 | 0.24 | −0.362 | 1.20E−05 | 8.62E−03 |
| Arpc5l | 8 | 0.381 | 0.254 | 0.383 | 2.08E−05 | 1.40E−02 |
| Slfn2 | 8 | 0.408 | 0.3 | 0.342 | 2.15E−05 | 1.45E−02 |
| Bcl2a1b | 8 | 0.151 | 0.215 | −0.507 | 2.23E−05 | 1.50E−02 |
| Rsrp1 | 8 | 0.559 | 0.436 | 0.318 | 2.24E−05 | 1.50E−02 |
| Il16 | 8 | 0.336 | 0.223 | 0.455 | 2.52E−05 | 1.68E−02 |
| Arf4 | 8 | 0.478 | 0.495 | −0.186 | 2.66E−05 | 1.76E−02 |
| Rab35 | 8 | 0.207 | 0.123 | 0.478 | 2.81E−05 | 1.85E−02 |
| Kdm2b | 8 | 0.158 | 0.203 | −0.379 | 3.08E−05 | 2.01E−02 |
| Ndel1 | 8 | 0.216 | 0.281 | −0.337 | 3.09E−05 | 2.02E−02 |
| Ppp3cc | 8 | 0.268 | 0.156 | 0.528 | 3.78E−05 | 2.41E−02 |
| Cdv3 | 8 | 0.255 | 0.294 | −0.281 | 4.42E−05 | 2.77E−02 |
| Nfkbie | 8 | 0.111 | 0.158 | −0.483 | 4.49E−05 | 2.82E−02 |
| Klf6 | 8 | 0.589 | 0.544 | 0.166 | 4.89E−05 | 3.04E−02 |
| Neat1 | 8 | 0.503 | 0.415 | 0.275 | 5.11E−05 | 3.15E−02 |
| Samd9l | 8 | 0.176 | 0.106 | 0.479 | 5.18E−05 | 3.19E−02 |
| Arrb2 | 8 | 0.32 | 0.246 | 0.295 | 5.19E−05 | 3.19E−02 |
| Jun | 8 | 0.453 | 0.384 | 0.210 | 5.29E−05 | 3.25E−02 |
| P2ry10 | 8 | 0.374 | 0.396 | −0.196 | 5.34E−05 | 3.27E−02 |
| Nfil3 | 8 | 0.365 | 0.244 | 0.490 | 5.82E−05 | 3.53E−02 |
| Ash1l | 8 | 0.235 | 0.139 | 0.491 | 5.93E−05 | 3.57E−02 |
| Hcst | 8 | 0.898 | 0.809 | 0.174 | 6.28E−05 | 3.75E−02 |
| Klf4 | 8 | 0.122 | 0.175 | −0.524 | 6.42E−05 | 3.82E−02 |
| Cap1 | 8 | 0.695 | 0.601 | 0.235 | 6.52E−05 | 3.86E−02 |
| Bhlhe40 | 8 | 0.571 | 0.62 | −0.159 | 6.54E−05 | 3.87E−02 |
| Lax1 | 8 | 0.286 | 0.18 | 0.486 | 7.05E−05 | 4.14E−02 |
| Clec2d | 8 | 0.289 | 0.194 | 0.391 | 7.08E−05 | 4.15E−02 |
| Cd69 | 8 | 0.306 | 0.357 | −0.386 | 7.60E−05 | 4.42E−02 |
| Samsn1 | 8 | 0.324 | 0.369 | −0.280 | 8.02E−05 | 4.63E−02 |
| Tuba4a | 8 | 0.191 | 0.222 | −0.361 | 8.56E−05 | 4.89E−02 |
| Igha | 9 | 0.566 | 0.317 | 0.227 | 2.42E−50 | 9.06E−46 |
| Ighg3 | 9 | 0.211 | 0.027 | 1.831 | 5.18E−28 | 4.69E−24 |
| Wdr89 | 9 | 0.993 | 0.989 | 0.469 | 4.18E−24 | 2.84E−20 |
| Zfp36l2 | 9 | 0.758 | 0.305 | 1.590 | 8.84E−23 | 5.48E−19 |
| Mcpt1 | 9 | 0.156 | 0.003 | 2.708 | 2.58E−21 | 1.46E−17 |
| Uba52 | 9 | 0.996 | 0.992 | 0.365 | 9.80E−13 | 2.26E−09 |
| Hspa8 | 9 | 0.995 | 0.998 | −0.426 | 4.24E−12 | 9.04E−09 |
| Rsrp1 | 9 | 0.796 | 0.802 | −0.274 | 1.03E−10 | 1.91E−07 |
| Tsc22d3 | 9 | 0.669 | 0.347 | 1.031 | 2.68E−10 | 4.54E−07 |
| Ddit4 | 9 | 0.241 | 0.105 | 0.791 | 7.45E−09 | 1.01E−05 |
| Nme2 | 9 | 0.4 | 0.228 | 0.703 | 1.33E−08 | 1.75E−05 |
| Serinc3 | 9 | 0.874 | 0.576 | 0.784 | 4.83E−08 | 5.82E−05 |
| Ubb | 9 | 0.998 | 1 | −0.190 | 4.77E−07 | 4.71E−04 |
| Basp1 | 9 | 0.871 | 0.947 | −0.559 | 1.11E−06 | 1.01E−03 |
| Il2 | 9 | 0.244 | 0.092 | 0.918 | 2.19E−06 | 1.90E−03 |
| Cdk1 | 9 | 0.478 | 0.555 | −0.396 | 3.14E−06 | 2.63E−03 |
| Cks1brt | 9 | 0.262 | 0.111 | 1.033 | 3.83E−06 | 3.13E−03 |
| Actg1 | 9 | 1 | 1 | −0.161 | 3.88E−06 | 3.16E−03 |
| Tubb5 | 9 | 0.966 | 0.973 | −0.244 | 4.06E−06 | 3.30E−03 |
| Malat1 | 9 | 1 | 0.998 | −0.269 | 4.52E−06 | 3.64E−03 |
| Lnx2 | 9 | 0.191 | 0.075 | 0.811 | 8.25E−06 | 6.18E−03 |
| Narf | 9 | 0.441 | 0.28 | 0.449 | 1.29E−05 | 9.17E−03 |
| Apobec1 | 9 | 0.86 | 0.753 | 0.201 | 3.28E−05 | 2.13E−02 |
| AC125149.3 | 9 | 0.619 | 0.477 | 0.342 | 5.12E−05 | 3.16E−02 |
| Eif2ak3 | 9 | 0.384 | 0.189 | 0.581 | 7.28E−05 | 4.25E−02 |
| Zfp36l2 | 10 | 0.68 | 0.182 | 1.856 | 5.31E−26 | 4.02E−22 |
| Igha | 10 | 0.405 | 0.229 | 0.162 | 2.16E−16 | 7.61E−13 |
| Tsc22d3 | 10 | 0.689 | 0.322 | 1.088 | 2.41E−14 | 6.57E−11 |
| Ttc7 | 10 | 0.244 | 0.061 | 1.338 | 4.72E−12 | 9.91E−09 |
| Sesn1 | 10 | 0.402 | 0.141 | 1.071 | 7.32E−11 | 1.37E−07 |
| 1110059E24Rik | 10 | 0.507 | 0.266 | 0.715 | 2.47E−10 | 4.25E−07 |
| Apobec1 | 10 | 0.807 | 0.618 | 0.309 | 1.04E−07 | 1.17E−04 |
| Ddit4 | 10 | 0.296 | 0.126 | 0.864 | 2.21E−07 | 2.34E−04 |
| Uba52 | 10 | 0.985 | 0.948 | 0.287 | 2.96E−07 | 3.05E−04 |
| Rsrp1 | 10 | 0.722 | 0.702 | −0.187 | 7.68E−07 | 7.23E−04 |
| Hspa8 | 10 | 0.975 | 0.984 | −0.286 | 3.38E−06 | 2.80E−03 |
| Dusp1 | 10 | 0.451 | 0.253 | 0.614 | 3.45E−06 | 2.85E−03 |
| Napsa | 10 | 0.776 | 0.644 | 0.374 | 4.92E−06 | 3.90E−03 |
| Cd47 | 10 | 0.602 | 0.368 | 0.638 | 6.15E−06 | 4.76E−03 |
| Egln2 | 10 | 0.558 | 0.321 | 0.624 | 7.51E−06 | 5.69E−03 |
| Btg1 | 10 | 0.985 | 0.95 | 0.438 | 1.03E−05 | 7.53E−03 |
| Serinc3 | 10 | 0.764 | 0.397 | 0.883 | 1.09E−05 | 7.93E−03 |
| Ppp1r21 | 10 | 0.165 | 0.052 | 1.045 | 1.38E−05 | 9.74E−03 |
| Crlf3 | 10 | 0.658 | 0.479 | 0.396 | 2.36E−05 | 1.59E−02 |
| Basp1 | 10 | 0.755 | 0.852 | −0.516 | 3.20E−05 | 2.08E−02 |
| Sh3glb2 | 10 | 0.187 | 0.134 | 0.383 | 6.45E−05 | 3.83E−02 |
| Jund | 11 | 0.855 | 0.944 | −0.624 | 3.24E−34 | 4.33E−30 |
| Id2 | 11 | 0.879 | 0.641 | 0.666 | 6.36E−27 | 5.11E−23 |
| Egr1 | 11 | 0.191 | 0.33 | −1.192 | 1.14E−17 | 4.52E−14 |
| Fabp2 | 11 | 0.021 | 0.151 | −2.142 | 3.09E−17 | 1.18E−13 |
| AY036118 | 11 | 0.447 | 0.432 | −0.357 | 5.25E−17 | 1.98E−13 |
| Crem | 11 | 0.344 | 0.551 | −0.785 | 7.48E−17 | 2.75E−13 |
| Nr4a1 | 11 | 0.625 | 0.775 | −0.424 | 7.83E−17 | 2.87E−13 |
| Junb | 11 | 0.938 | 0.981 | −0.194 | 2.59E−16 | 9.03E−13 |
| Nr4a3 | 11 | 0.195 | 0.378 | −0.968 | 8.68E−16 | 2.84E−12 |
| Mcpt1 | 11 | 0.217 | 0.041 | 1.668 | 1.94E−14 | 5.45E−11 |
| Pim1 | 11 | 0.799 | 0.92 | −0.269 | 2.06E−14 | 5.72E−11 |
| Txnip | 11 | 0.434 | 0.21 | 0.976 | 2.08E−14 | 5.75E−11 |
| Kdm6b | 11 | 0.346 | 0.499 | −0.703 | 4.22E−14 | 1.14E−10 |
| Litaf | 11 | 0.387 | 0.553 | −0.562 | 5.84E−13 | 1.40E−09 |
| Tgif1 | 11 | 0.408 | 0.575 | −0.505 | 8.44E−13 | 1.98E−09 |
| Vps37b | 11 | 0.732 | 0.829 | −0.394 | 1.30E−12 | 2.95E−09 |
| Ier5l | 11 | 0.047 | 0.162 | −1.609 | 1.53E−12 | 3.43E−09 |
| Rel | 11 | 0.195 | 0.361 | −0.837 | 2.10E−12 | 4.67E−09 |
| Zfp36l2 | 11 | 0.803 | 0.6 | 0.464 | 2.16E−12 | 4.76E−09 |
| Nr4a2 | 11 | 0.236 | 0.4 | −0.777 | 3.96E−12 | 8.47E−09 |
| Lmna | 11 | 0.074 | 0.19 | −1.198 | 4.31E−12 | 9.15E−09 |
| Nfkbid | 11 | 0.287 | 0.46 | −0.759 | 4.43E−12 | 9.36E−09 |
| Ier2 | 11 | 0.707 | 0.814 | −0.331 | 4.56E−12 | 9.60E−09 |
| Traf1 | 11 | 0.57 | 0.698 | −0.462 | 5.06E−12 | 1.06E−08 |
| Defa24 | 11 | 0.035 | 0.16 | −1.545 | 1.58E−11 | 3.17E−08 |
| Phlda1 | 11 | 0.205 | 0.376 | −0.883 | 1.59E−11 | 3.18E−08 |
| Rgs1 | 11 | 0.857 | 0.711 | 0.486 | 2.93E−11 | 5.70E−08 |
| Nfkb1 | 11 | 0.301 | 0.419 | −0.649 | 3.42E−11 | 6.58E−08 |
| Igkc | 11 | 0.691 | 0.508 | 0.474 | 3.59E−11 | 6.90E−08 |
| Klrc1 | 11 | 0.676 | 0.505 | 0.477 | 8.20E−11 | 1.53E−07 |
| Tpm4 | 11 | 0.453 | 0.583 | −0.477 | 1.04E−10 | 1.91E−07 |
| Fam110a | 11 | 0.105 | 0.22 | −0.925 | 2.58E−10 | 4.40E−07 |
| Fosl2 | 11 | 0.35 | 0.492 | −0.566 | 3.03E−10 | 5.08E−07 |
| Nfkbiz | 11 | 0.545 | 0.665 | −0.342 | 5.04E−10 | 8.21E−07 |
| Irf8 | 11 | 0.447 | 0.553 | −0.411 | 6.67E−10 | 1.07E−06 |
| Maff | 11 | 0.404 | 0.562 | −0.400 | 7.21E−10 | 1.16E−06 |
| Rora | 11 | 0.5 | 0.603 | −0.466 | 8.69E−10 | 1.38E−06 |
| Sla | 11 | 0.498 | 0.274 | 0.612 | 1.41E−09 | 2.15E−06 |
| Ifrd1 | 11 | 0.32 | 0.458 | −0.514 | 2.53E−09 | 3.72E−06 |
| Klrb1f | 11 | 0.656 | 0.495 | 0.457 | 9.35E−09 | 1.25E−05 |
| Gadd45b | 11 | 0.447 | 0.598 | −0.389 | 1.26E−08 | 1.65E−05 |
| Ikzf2 | 11 | 0.721 | 0.801 | −0.383 | 1.39E−08 | 1.82E−05 |
| Bcl2a1d | 11 | 0.607 | 0.683 | −0.405 | 1.45E−08 | 1.88E−05 |
| Hist1h1c | 11 | 0.402 | 0.21 | 0.662 | 1.81E−08 | 2.32E−05 |
| Bcl2 | 11 | 0.916 | 0.814 | 0.380 | 1.82E−08 | 2.33E−05 |
| Tsc22d3 | 11 | 0.582 | 0.393 | 0.564 | 1.97E−08 | 2.51E−05 |
| Ptp4a2 | 11 | 0.652 | 0.739 | −0.322 | 2.31E−08 | 2.92E−05 |
| Nfkbia | 11 | 0.82 | 0.892 | −0.211 | 2.33E−08 | 2.95E−05 |
| Dusp5 | 11 | 0.691 | 0.771 | −0.198 | 3.02E−08 | 3.76E−05 |
| Bcl2a1b | 11 | 0.518 | 0.6 | −0.492 | 5.15E−08 | 6.17E−05 |
| Sik1 | 11 | 0.146 | 0.231 | −0.679 | 6.10E−08 | 7.18E−05 |
| Il7r | 11 | 0.824 | 0.734 | 0.359 | 7.33E−08 | 8.43E−05 |
| Nabp1 | 11 | 0.754 | 0.771 | −0.335 | 7.69E−08 | 8.82E−05 |
| Cd74 | 11 | 0.498 | 0.581 | −0.289 | 1.12E−07 | 1.24E−04 |
| Btg2 | 11 | 0.947 | 0.976 | −0.166 | 1.57E−07 | 1.72E−04 |
| Sub1 | 11 | 0.898 | 0.94 | −0.239 | 2.03E−07 | 2.17E−04 |
| Coq10b | 11 | 0.287 | 0.406 | −0.527 | 2.42E−07 | 2.54E−04 |
| Pnrc1 | 11 | 0.826 | 0.89 | −0.214 | 2.63E−07 | 2.74E−04 |
| Skil | 11 | 0.166 | 0.281 | −0.626 | 3.25E−07 | 3.32E−04 |
| Samsn1 | 11 | 0.455 | 0.54 | −0.404 | 3.54E−07 | 3.57E−04 |
| Ltb | 11 | 0.975 | 0.946 | 0.288 | 4.72E−07 | 4.66E−04 |
| Ubb | 11 | 0.998 | 1 | −0.186 | 5.50E−07 | 5.34E−04 |
| Ahr | 11 | 0.395 | 0.248 | 0.630 | 5.85E−07 | 5.64E−04 |
| Mir142hg | 11 | 0.148 | 0.253 | −0.656 | 6.33E−07 | 6.07E−04 |
| Map3k14 | 11 | 0.141 | 0.24 | −0.737 | 8.20E−07 | 7.65E−04 |
| Cited4 | 11 | 0.512 | 0.346 | 0.292 | 9.52E−07 | 8.81E−04 |
| Gimap9 | 11 | 0.5 | 0.354 | 0.467 | 3.22E−06 | 2.69E−03 |
| Gimap6 | 11 | 0.854 | 0.749 | 0.279 | 3.36E−06 | 2.79E−03 |
| Gimap7 | 11 | 0.719 | 0.59 | 0.349 | 3.56E−06 | 2.94E−03 |
| Dok1 | 11 | 0.215 | 0.102 | 0.756 | 3.75E−06 | 3.07E−03 |
| B2m | 11 | 0.998 | 1 | 0.156 | 4.36E−06 | 3.54E−03 |
| Zc3h12a | 11 | 0.246 | 0.363 | −0.506 | 4.37E−06 | 3.54E−03 |
| AW112010 | 11 | 1 | 0.994 | 0.167 | 5.49E−06 | 4.32E−03 |
| Igha | 11 | 0.461 | 0.283 | 0.479 | 5.99E−06 | 4.66E−03 |
| Gnb1 | 11 | 0.32 | 0.419 | −0.404 | 7.40E−06 | 5.61E−03 |
| Hexim1 | 11 | 0.09 | 0.175 | −0.822 | 7.63E−06 | 5.75E−03 |
| Gimap3 | 11 | 0.941 | 0.914 | 0.224 | 7.97E−06 | 5.98E−03 |
| Rhog | 11 | 0.213 | 0.313 | −0.491 | 1.00E−05 | 7.37E−03 |
| Bhlhe40 | 11 | 0.623 | 0.693 | −0.227 | 1.05E−05 | 7.66E−03 |
| Tmem176a | 11 | 0.701 | 0.536 | 0.383 | 1.47E−05 | 1.03E−02 |
| Tsix | 11 | 0.133 | 0.201 | −0.615 | 1.53E−05 | 1.07E−02 |
| VgIl4 | 11 | 0.387 | 0.479 | −0.446 | 1.82E−05 | 1.25E−02 |
| Gimap1 | 11 | 0.754 | 0.652 | 0.275 | 2.92E−05 | 1.91E−02 |
| Gna13 | 11 | 0.371 | 0.449 | −0.345 | 3.23E−05 | 2.10E−02 |
| Cd69 | 11 | 0.496 | 0.598 | −0.200 | 3.41E−05 | 2.20E−02 |
| Amd1 | 11 | 0.174 | 0.238 | −0.549 | 3.57E−05 | 2.29E−02 |
| Ngrn | 11 | 0.168 | 0.067 | 0.921 | 4.08E−05 | 2.58E−02 |
| Bud31 | 11 | 0.291 | 0.367 | −0.400 | 4.56E−05 | 2.85E−02 |
| Ubc | 11 | 0.369 | 0.464 | −0.354 | 4.82E−05 | 3.01E−02 |
| Gimap5 | 11 | 0.672 | 0.529 | 0.360 | 4.84E−05 | 3.01E−02 |
| 9930111J21Rik2 | 11 | 0.217 | 0.106 | 0.791 | 4.94E−05 | 3.06E−02 |
| Icam1 | 11 | 0.281 | 0.38 | −0.441 | 5.73E−05 | 3.49E−02 |
| Mxd1 | 11 | 0.523 | 0.611 | −0.273 | 6.08E−05 | 3.64E−02 |
| Asb2 | 11 | 0.441 | 0.294 | 0.482 | 6.95E−05 | 4.09E−02 |
| Eif4a1 | 11 | 0.727 | 0.754 | −0.192 | 7.81E−05 | 4.52E−02 |
| Anapc16 | 11 | 0.195 | 0.097 | 0.702 | 8.29E−05 | 4.76E−02 |
| Srgn | 12 | 0.99 | 0.876 | 0.838 | 6.70E−42 | 1.39E−37 |
| Il13 | 12 | 0.614 | 0.233 | 2.088 | 1.02E−33 | 1.30E−29 |
| Plk3 | 12 | 0.419 | 0.066 | 2.118 | 5.52E−29 | 5.24E−25 |
| Sla | 12 | 0.73 | 0.342 | 1.237 | 2.30E−25 | 1.66E−21 |
| Zfp36l1 | 12 | 0.984 | 0.922 | 0.569 | 4.00E−24 | 2.74E−20 |
| Jund | 12 | 0.888 | 0.949 | −0.733 | 1.05E−23 | 6.98E−20 |
| Hspa8 | 12 | 0.974 | 0.894 | 0.424 | 2.23E−22 | 1.36E−18 |
| Il4 | 12 | 0.701 | 0.352 | 1.591 | 3.24E−22 | 1.93E−18 |
| Cxcr6 | 12 | 0.884 | 0.684 | 0.732 | 1.40E−21 | 7.98E−18 |
| Furin | 12 | 0.789 | 0.504 | 1.089 | 4.44E−21 | 2.44E−17 |
| Ier2 | 12 | 0.785 | 0.823 | −0.913 | 1.29E−20 | 6.96E−17 |
| Pim3 | 12 | 0.551 | 0.241 | 1.184 | 4.81E−20 | 2.54E−16 |
| Nab2 | 12 | 0.309 | 0.046 | 2.136 | 1.70E−19 | 8.27E−16 |
| Areg | 12 | 0.589 | 0.271 | 1.194 | 3.57E−18 | 1.54E−14 |
| Rel | 12 | 0.476 | 0.185 | 1.320 | 9.56E−18 | 3.86E−14 |
| Ubb | 12 | 0.984 | 0.967 | −0.307 | 1.83E−17 | 7.06E−14 |
| Egr1 | 12 | 0.449 | 0.489 | −0.834 | 3.18E−16 | 1.09E−12 |
| Fos | 12 | 0.797 | 0.868 | −0.975 | 4.46E−16 | 1.49E−12 |
| Zap70 | 12 | 0.604 | 0.281 | 0.945 | 5.90E−16 | 1.95E−12 |
| Rheb | 12 | 0.738 | 0.458 | 0.668 | 9.15E−16 | 2.98E−12 |
| Dusp1 | 12 | 0.734 | 0.825 | −0.671 | 1.04E−15 | 3.33E−12 |
| Cytip | 12 | 0.858 | 0.572 | 0.629 | 2.35E−15 | 7.19E−12 |
| Lif | 12 | 0.348 | 0.099 | 1.263 | 2.71E−15 | 8.28E−12 |
| Lmna | 12 | 0.085 | 0.253 | −1.359 | 8.70E−15 | 2.53E−11 |
| Hilpda | 12 | 0.921 | 0.785 | 0.484 | 1.55E−13 | 3.99E−10 |
| Cycs | 12 | 0.695 | 0.42 | 0.799 | 2.60E−13 | 6.60E−10 |
| Sptssa | 12 | 0.898 | 0.909 | −0.295 | 2.64E−13 | 6.69E−10 |
| Nxpe3 | 12 | 0.307 | 0.076 | 1.364 | 2.71E−13 | 6.83E−10 |
| Mcpt1 | 12 | 0.242 | 0.048 | 1.458 | 3.95E−13 | 9.74E−10 |
| Prr7 | 12 | 0.118 | 0.263 | −1.304 | 8.63E−13 | 2.01E−09 |
| Pdcd1 | 12 | 0.443 | 0.147 | 1.179 | 1.90E−12 | 4.22E−09 |
| Fabp2 | 12 | 0.024 | 0.157 | −2.073 | 5.42E−12 | 1.13E−08 |
| Rap1b | 12 | 0.76 | 0.524 | 0.609 | 1.52E−11 | 3.05E−08 |
| Klf2 | 12 | 0.394 | 0.509 | −1.024 | 2.39E−11 | 4.67E−08 |
| Gadd45b | 12 | 0.807 | 0.58 | 0.474 | 8.59E−11 | 1.60E−07 |
| Nrgn | 12 | 0.427 | 0.18 | 1.027 | 1.20E−10 | 2.18E−07 |
| Adora2a | 12 | 0.398 | 0.162 | 1.022 | 1.20E−10 | 2.18E−07 |
| Plaur | 12 | 0.447 | 0.175 | 1.074 | 1.32E−10 | 2.38E−07 |
| Spty2d1 | 12 | 0.713 | 0.499 | 0.568 | 1.86E−10 | 3.28E−07 |
| Nr4a2 | 12 | 0.526 | 0.294 | 0.760 | 2.58E−10 | 4.40E−07 |
| Ccl1 | 12 | 0.285 | 0.101 | 1.634 | 2.93E−10 | 4.95E−07 |
| Kif23 | 12 | 0.138 | 0.268 | −1.060 | 2.98E−10 | 5.01E−07 |
| Cd27 | 12 | 0.693 | 0.468 | 0.632 | 4.81E−10 | 7.88E−07 |
| Lgals7 | 12 | 0.215 | 0.051 | 1.450 | 7.68E−10 | 1.23E−06 |
| Gypc | 12 | 0.358 | 0.142 | 0.911 | 8.21E−10 | 1.31E−06 |
| Bcl2 | 12 | 0.791 | 0.625 | 0.473 | 9.98E−10 | 1.57E−06 |
| Slc3a2 | 12 | 0.72 | 0.468 | 0.577 | 1.64E−09 | 2.49E−06 |
| Cisd2 | 12 | 0.638 | 0.337 | 0.655 | 1.85E−09 | 2.78E−06 |
| Abi3 | 12 | 0.159 | 0.294 | −0.824 | 2.43E−09 | 3.59E−06 |
| Defa24 | 12 | 0.061 | 0.182 | −1.210 | 2.49E−09 | 3.66E−06 |
| Cotl1 | 12 | 0.774 | 0.775 | −0.219 | 4.37E−09 | 6.24E−06 |
| Tnfaip8 | 12 | 0.638 | 0.385 | 0.663 | 4.69E−09 | 6.64E−06 |
| Gata3 | 12 | 0.982 | 0.901 | 0.334 | 5.32E−09 | 7.47E−06 |
| Clk1 | 12 | 0.427 | 0.542 | −0.574 | 5.66E−09 | 7.93E−06 |
| Dnaja1 | 12 | 0.904 | 0.901 | −0.457 | 6.32E−09 | 8.79E−06 |
| Ccr4 | 12 | 0.407 | 0.177 | 0.845 | 6.35E−09 | 8.82E−06 |
| Nfkb1 | 12 | 0.884 | 0.772 | 0.412 | 6.68E−09 | 9.22E−06 |
| Klf6 | 12 | 0.441 | 0.557 | −0.873 | 6.76E−09 | 9.31E−06 |
| Tgfb1 | 12 | 0.618 | 0.405 | 0.679 | 8.22E−09 | 1.11E−05 |
| Lilrb4a | 12 | 0.313 | 0.127 | 1.020 | 1.05E−08 | 1.40E−05 |
| Mmd | 12 | 0.28 | 0.076 | 1.490 | 1.41E−08 | 1.84E−05 |
| Ifngr2 | 12 | 0.376 | 0.167 | 0.916 | 1.46E−08 | 1.89E−05 |
| Id2 | 12 | 0.878 | 0.724 | 0.583 | 1.99E−08 | 2.53E−05 |
| Prdx6 | 12 | 0.726 | 0.506 | 0.666 | 2.46E−08 | 3.09E−05 |
| Serpina3g | 12 | 0.467 | 0.294 | 1.116 | 2.51E−08 | 3.14E−05 |
| Trf | 12 | 0.222 | 0.066 | 1.243 | 4.52E−08 | 5.46E−05 |
| Rassf5 | 12 | 0.455 | 0.205 | 0.796 | 4.94E−08 | 5.93E−05 |
| Gch1 | 12 | 0.157 | 0.028 | 1.674 | 5.78E−08 | 6.87E−05 |
| Gpcpd1 | 12 | 0.254 | 0.38 | −0.513 | 5.86E−08 | 6.91E−05 |
| Cited2 | 12 | 0.325 | 0.486 | −0.576 | 6.19E−08 | 7.26E−05 |
| Crem | 12 | 0.713 | 0.484 | 0.281 | 6.98E−08 | 8.08E−05 |
| Dtx1 | 12 | 0.268 | 0.408 | −0.557 | 7.14E−08 | 8.24E−05 |
| Gimap5 | 12 | 0.553 | 0.322 | 0.704 | 9.51E−08 | 1.08E−04 |
| Eef2 | 12 | 0.982 | 0.942 | 0.321 | 1.01E−07 | 1.14E−04 |
| Abhd17c | 12 | 0.465 | 0.248 | 0.667 | 1.02E−07 | 1.15E−04 |
| Nfkbiz | 12 | 0.874 | 0.737 | 0.340 | 1.04E−07 | 1.17E−04 |
| Tagln2 | 12 | 0.74 | 0.709 | −0.161 | 1.22E−07 | 1.35E−04 |
| Sh2d2a | 12 | 0.533 | 0.271 | 0.767 | 1.33E−07 | 1.46E−04 |
| Egr2 | 12 | 0.157 | 0.03 | 1.855 | 1.65E−07 | 1.80E−04 |
| Fosl2 | 12 | 0.677 | 0.456 | 0.481 | 1.73E−07 | 1.88E−04 |
| Ppp1r15a | 12 | 0.654 | 0.709 | −0.415 | 2.01E−07 | 2.15E−04 |
| Selplg | 12 | 0.862 | 0.706 | 0.461 | 2.20E−07 | 2.34E−04 |
| Atp2b1 | 12 | 0.524 | 0.311 | 0.573 | 2.69E−07 | 2.80E−04 |
| Eif4a1 | 12 | 0.911 | 0.775 | 0.421 | 2.98E−07 | 3.07E−04 |
| Rhob | 12 | 0.234 | 0.322 | −0.776 | 3.43E−07 | 3.47E−04 |
| Scp2 | 12 | 0.65 | 0.646 | −0.199 | 3.49E−07 | 3.52E−04 |
| Rap1a | 12 | 0.553 | 0.319 | 0.726 | 3.78E−07 | 3.79E−04 |
| Zfp36 | 12 | 0.514 | 0.491 | −0.561 | 4.30E−07 | 4.28E−04 |
| Hic1 | 12 | 0.356 | 0.144 | 0.964 | 5.04E−07 | 4.94E−04 |
| Tpm4 | 12 | 0.551 | 0.329 | 0.654 | 5.60E−07 | 5.41E−04 |
| Esm1 | 12 | 0.093 | 0.233 | −0.997 | 6.47E−07 | 6.18E−04 |
| Kcnn4 | 12 | 0.234 | 0.309 | −0.527 | 7.30E−07 | 6.91E−04 |
| Orai1 | 12 | 0.459 | 0.238 | 0.758 | 1.01E−06 | 9.32E−04 |
| Ubc | 12 | 0.404 | 0.489 | −0.472 | 1.04E−06 | 9.54E−04 |
| Txnip | 12 | 0.114 | 0.218 | −1.091 | 1.05E−06 | 9.57E−04 |
| Fosb | 12 | 0.472 | 0.496 | −0.561 | 1.54E−06 | 1.37E−03 |
| Cntnap2 | 12 | 0.083 | 0.187 | −1.029 | 1.99E−06 | 1.75E−03 |
| Nab1 | 12 | 0.246 | 0.084 | 1.142 | 2.04E−06 | 1.78E−03 |
| Traf4 | 12 | 0.301 | 0.162 | 0.878 | 2.66E−06 | 2.28E−03 |
| AY036118 | 12 | 0.429 | 0.438 | −0.381 | 2.85E−06 | 2.41E−03 |
| Igkc | 12 | 0.734 | 0.547 | 0.321 | 3.30E−06 | 2.76E−03 |
| Tsc22d3 | 12 | 0.333 | 0.375 | −0.528 | 3.31E−06 | 2.76E−03 |
| Rasa2 | 12 | 0.354 | 0.162 | 0.780 | 3.32E−06 | 2.77E−03 |
| Igha | 12 | 0.516 | 0.316 | 0.508 | 3.35E−06 | 2.78E−03 |
| Nr4a3 | 12 | 0.559 | 0.37 | 0.596 | 3.41E−06 | 2.82E−03 |
| Sub1 | 12 | 0.978 | 0.919 | 0.258 | 3.46E−06 | 2.86E−03 |
| Jun | 12 | 0.616 | 0.689 | −0.758 | 3.79E−06 | 3.10E−03 |
| DynIl1 | 12 | 0.691 | 0.499 | 0.524 | 3.80E−06 | 3.11E−03 |
| Il2ra | 12 | 0.604 | 0.382 | 0.561 | 4.84E−06 | 3.85E−03 |
| Ramp1 | 12 | 0.417 | 0.435 | −0.296 | 5.11E−06 | 4.04E−03 |
| Mapkapk2 | 12 | 0.211 | 0.066 | 1.193 | 5.83E−06 | 4.55E−03 |
| Slc2a3 | 12 | 0.157 | 0.043 | 1.265 | 6.66E−06 | 5.12E−03 |
| Btg3 | 12 | 0.266 | 0.104 | 0.862 | 6.70E−06 | 5.14E−03 |
| Arl5c | 12 | 0.179 | 0.271 | −0.886 | 7.34E−06 | 5.57E−03 |
| Lilr4b | 12 | 0.274 | 0.111 | 0.886 | 7.85E−06 | 5.90E−03 |
| Ndfip1 | 12 | 0.417 | 0.241 | 0.606 | 9.38E−06 | 6.95E−03 |
| Ptpn22 | 12 | 0.451 | 0.271 | 0.629 | 9.54E−06 | 7.05E−03 |
| Tmem59 | 12 | 0.65 | 0.623 | −0.162 | 1.10E−05 | 8.01E−03 |
| Pnrc1 | 12 | 0.768 | 0.787 | −0.254 | 1.11E−05 | 8.07E−03 |
| Phf6 | 12 | 0.278 | 0.127 | 0.764 | 1.23E−05 | 8.77E−03 |
| H2-Q6 | 12 | 0.317 | 0.435 | −0.578 | 1.40E−05 | 9.90E−03 |
| Satb1 | 12 | 0.579 | 0.37 | 0.496 | 1.41E−05 | 9.95E−03 |
| Itk | 12 | 0.685 | 0.524 | 0.442 | 1.43E−05 | 1.01E−02 |
| Fgl2 | 12 | 0.695 | 0.468 | 0.408 | 1.91E−05 | 1.31E−02 |
| Hccs | 12 | 0.26 | 0.094 | 0.922 | 1.97E−05 | 1.34E−02 |
| Klf4 | 12 | 0.461 | 0.552 | −0.505 | 2.38E−05 | 1.60E−02 |
| Gnai2 | 12 | 0.593 | 0.625 | −0.303 | 2.56E−05 | 1.70E−02 |
| Ero1l | 12 | 0.455 | 0.251 | 0.822 | 2.57E−05 | 1.71E−02 |
| Mctp1 | 12 | 0.096 | 0.162 | −0.729 | 2.67E−05 | 1.76E−02 |
| Hes1 | 12 | 0.754 | 0.795 | −0.548 | 3.77E−05 | 2.41E−02 |
| Spry2 | 12 | 0.398 | 0.238 | 0.353 | 3.92E−05 | 2.49E−02 |
| Tnfrsf1b | 12 | 0.268 | 0.124 | 0.797 | 4.02E−05 | 2.55E−02 |
| Nfatc1 | 12 | 0.274 | 0.147 | 0.680 | 5.51E−05 | 3.36E−02 |
| Fam105a | 12 | 0.445 | 0.256 | 0.498 | 5.70E−05 | 3.47E−02 |
| Lpar6 | 12 | 0.285 | 0.354 | −0.494 | 5.81E−05 | 3.53E−02 |
| Serp1 | 12 | 0.807 | 0.8 | −0.216 | 6.34E−05 | 3.77E−02 |
| Psma6 | 12 | 0.291 | 0.266 | −0.175 | 6.91E−05 | 4.07E−02 |
| Vamp2 | 12 | 0.283 | 0.134 | 0.706 | 7.05E−05 | 4.14E−02 |
| March7 | 12 | 0.278 | 0.127 | 0.738 | 7.22E−05 | 4.22E−02 |
| Nfkbie | 12 | 0.1 | 0.175 | −0.749 | 7.56E−05 | 4.41E−02 |
| Lrp10 | 12 | 0.39 | 0.18 | 0.808 | 7.58E−05 | 4.41E−02 |
| Ssbp3 | 12 | 0.244 | 0.296 | −0.425 | 7.84E−05 | 4.53E−02 |
| Plscr1 | 12 | 0.463 | 0.301 | 0.580 | 8.32E−05 | 4.77E−02 |
| Nfkbid | 12 | 0.506 | 0.337 | 0.422 | 8.52E−05 | 4.87E−02 |
| Tpt1 | 12 | 1 | 1 | 0.182 | 8.70E−05 | 4.94E−02 |
| Wdr89 | 13 | 0.812 | 0.676 | 0.488 | 2.53E−12 | 5.48E−09 |
| Uba52 | 13 | 0.753 | 0.655 | 0.423 | 1.79E−07 | 1.93E−04 |
| Mcpt1 | 14 | 0.367 | 0.057 | 1.837 | 3.69E−21 | 2.05E−17 |
| Mcpt2 | 14 | 0.234 | 0.017 | 2.435 | 6.32E−18 | 2.63E−14 |
| Lyz1 | 14 | 0.05 | 0.24 | −0.177 | 6.49E−14 | 1.71E−10 |
| Defa24 | 14 | 0.106 | 0.333 | −1.055 | 8.53E−13 | 1.99E−09 |
| Grasp | 14 | 0.633 | 0.77 | −0.498 | 1.36E−12 | 3.09E−09 |
| Id2 | 14 | 0.702 | 0.584 | 0.543 | 8.97E−09 | 1.21E−05 |
| Nrros | 14 | 0.578 | 0.325 | 0.796 | 1.23E−08 | 1.62E−05 |
| Fabp2 | 14 | 0.046 | 0.19 | −1.518 | 5.79E−08 | 6.87E−05 |
| Rgs1 | 14 | 0.615 | 0.451 | 0.651 | 1.50E−07 | 1.65E−04 |
| Ifrd1 | 14 | 0.674 | 0.751 | −0.449 | 5.16E−07 | 5.05E−04 |
| Sub1 | 14 | 1 | 0.988 | −0.155 | 1.34E−06 | 1.20E−03 |
| Cfp | 14 | 0.904 | 0.91 | −0.366 | 2.85E−06 | 2.41E−03 |
| Maff | 14 | 0.647 | 0.77 | −0.329 | 3.56E−06 | 2.94E−03 |
| Igkc | 14 | 0.917 | 0.827 | −1.522 | 4.43E−06 | 3.58E−03 |
| Jund | 14 | 0.94 | 0.95 | −0.363 | 5.67E−06 | 4.44E−03 |
| Anxa3 | 14 | 0.083 | 0.242 | −1.191 | 1.00E−05 | 7.37E−03 |
| Plac8 | 14 | 0.821 | 0.651 | 0.297 | 1.47E−05 | 1.03E−02 |
| Plscr1 | 14 | 0.307 | 0.466 | −0.634 | 1.47E−05 | 1.03E−02 |
| Elf4 | 14 | 0.239 | 0.114 | 0.665 | 1.70E−05 | 1.17E−02 |
| Igha | 14 | 0.784 | 0.599 | −0.973 | 1.97E−05 | 1.34E−02 |
| AY036118 | 14 | 0.436 | 0.451 | −0.392 | 2.01E−05 | 1.36E−02 |
| Smap2 | 14 | 0.555 | 0.727 | −0.418 | 2.43E−05 | 1.63E−02 |
| Tbc1d4 | 14 | 0.761 | 0.81 | −0.261 | 2.60E−05 | 1.73E−02 |
| Ncl | 14 | 0.872 | 0.867 | −0.226 | 2.70E−05 | 1.78E−02 |
| Elmsan1 | 14 | 0.193 | 0.323 | −0.552 | 3.16E−05 | 2.06E−02 |
| Arf4 | 14 | 0.775 | 0.824 | −0.232 | 4.43E−05 | 2.78E−02 |
| Ccr1 | 14 | 0.197 | 0.382 | −0.803 | 4.54E−05 | 2.84E−02 |
| Polr3c | 14 | 0.298 | 0.173 | 0.494 | 4.90E−05 | 3.04E−02 |
| Trps1 | 14 | 0.505 | 0.644 | −0.415 | 6.20E−05 | 3.71E−02 |
| Bex6 | 14 | 0.188 | 0.067 | 1.241 | 7.11E−05 | 4.16E−02 |
| Birc2 | 14 | 0.252 | 0.354 | −0.642 | 8.12E−05 | 4.68E−02 |
| Tuba1c | 14 | 0.367 | 0.368 | −0.242 | 8.44E−05 | 4.83E−02 |
| Jund | 15 | 0.696 | 0.871 | −0.886 | 1.14E−10 | 2.08E−07 |
| Kdm6b | 15 | 0.28 | 0.575 | −0.851 | 2.25E−10 | 3.92E−07 |
| Hk2 | 15 | 0.096 | 0.305 | −1.074 | 1.32E−05 | 9.39E−03 |
| Lmna | 15 | 0.056 | 0.239 | −1.137 | 1.33E−05 | 9.40E−03 |
| Gimap6 | 15 | 0.48 | 0.333 | 0.667 | 2.10E−05 | 1.42E−02 |
| Pnrc1 | 15 | 0.424 | 0.635 | −0.562 | 3.36E−05 | 2.17E−02 |
| Pim1 | 15 | 0.496 | 0.71 | −0.599 | 6.51E−05 | 3.86E−02 |
| Id2 | 15 | 0.728 | 0.514 | 0.579 | 8.76E−05 | 4.96E−02 |
| Hes1 | 16 | 0.084 | 0.193 | −1.843 | 5.36E−07 | 5.21E−04 |
| Gpcpd1 | 16 | 0.118 | 0.22 | −0.962 | 2.33E−06 | 2.02E−03 |
| Dnaja1 | 16 | 0.382 | 0.487 | −0.735 | 7.82E−06 | 5.88E−03 |
| Ppp1r15a | 16 | 0.189 | 0.293 | −1.206 | 1.91E−05 | 1.31E−02 |
| Sla | 16 | 0.412 | 0.173 | 1.197 | 2.54E−05 | 1.70E−02 |
| Cdk2ap2 | 16 | 0.08 | 0.167 | −1.073 | 4.59E−05 | 2.87E−02 |
| Gpr183 | 16 | 0.424 | 0.347 | −0.256 | 8.06E−05 | 4.65E−02 |
| Ptma | 16 | 0.471 | 0.533 | −0.390 | 8.17E−05 | 4.70E−02 |
| Chchd10 | 17 | 0.235 | 0.11 | 0.792 | 5.97E−05 | 3.59E−02 |
| Wdr89 | 18 | 1 | 0.96 | 0.510 | 1.74E−07 | 1.88E−04 |
| Wsb1 | 18 | 0.038 | 0.169 | −1.529 | 1.07E−05 | 7.83E−03 |
| Junb | 19 | 0.774 | 0.737 | −0.245 | 2.79E−06 | 2.37E−03 |
| Mcpt1 | 20 | 0.45 | 0.048 | 2.227 | 2.11E−10 | 3.69E−07 |
| Mcpt2 | 20 | 0.321 | 0.01 | 3.407 | 1.02E−08 | 1.35E−05 |
| Fabp2 | 20 | 0.061 | 0.276 | −2.260 | 2.15E−06 | 1.87E−03 |
| Lyz1 | 20 | 0.038 | 0.2 | −2.380 | 1.19E−05 | 8.58E−03 |
| Mcpt1 | 21 | 0.427 | 0.06 | 3.356 | 3.40E−09 | 4.92E−06 |
| H2afz | 21 | 0.983 | 0.991 | −0.307 | 3.72E−06 | 3.06E−03 |
| Defa24 | 21 | 0.248 | 0.444 | −0.656 | 9.88E−06 | 7.29E−03 |
| Ly6e | 21 | 0.65 | 0.752 | −0.726 | 1.02E−05 | 7.47E−03 |
| Efhd2 | 21 | 0.889 | 0.915 | −0.342 | 2.03E−05 | 1.37E−02 |
| Nrros | 21 | 0.658 | 0.436 | 0.861 | 2.90E−05 | 1.90E−02 |
| Sla | 21 | 0.863 | 0.744 | 0.496 | 3.49E−05 | 2.24E−02 |
| Twf2 | 21 | 0.838 | 0.667 | 0.477 | 6.58E−05 | 3.89E−02 |
| Hspa1a | 21 | 0.556 | 0.47 | 0.298 | 7.00E−05 | 4.11E−02 |
| Jund | 22 | 0.898 | 0.947 | −0.710 | 2.21E−07 | 2.34E−04 |
| Gapdh | 22 | 0.873 | 0.8 | 0.582 | 1.58E−06 | 1.40E−03 |
| Trpv2 | 22 | 0.197 | 0 | 3.860 | 3.81E−06 | 3.11E−03 |
| Pla2g2d | 22 | 0.032 | 0.187 | −2.738 | 3.41E−05 | 2.20E−02 |
| Fam213b | 22 | 0.051 | 0.173 | −1.795 | 4.19E−05 | 2.65E−02 |
| Hspa8 | 22 | 0.987 | 0.973 | 0.298 | 5.00E−05 | 3.09E−02 |
| Lgals3bp | 22 | 0.325 | 0.293 | −0.597 | 5.93E−05 | 3.57E−02 |
| Igha | 24 | 0.688 | 0.263 | 1.262 | 6.96E−12 | 1.44E−08 |
| Lyz1 | 24 | 0 | 0.175 | −3.978 | 8.81E−07 | 8.17E−04 |
| Mcpt1 | 24 | 0.257 | 0.035 | 3.554 | 1.05E−06 | 9.59E−04 |
| Mcpt2 | 24 | 0.211 | 0.009 | 4.485 | 4.73E−06 | 3.78E−03 |
| Arpp19 | 24 | 0.872 | 0.632 | 0.513 | 3.29E−05 | 2.13E−02 |
| Ppp2r2d | 24 | 0.083 | 0.254 | −0.941 | 5.57E−05 | 3.40E−02 |
| Egfl7 | 25 | 0.895 | 0.817 | 0.621 | 1.95E−07 | 2.09E−04 |
| 8430408G22Rik | 25 | 0.702 | 0.341 | 1.076 | 4.38E−06 | 3.54E−03 |
| Hspa1b | 25 | 0.193 | 0.098 | 1.118 | 6.67E−06 | 5.12E−03 |
| Fzd4 | 25 | 0.368 | 0.14 | 0.813 | 5.61E−05 | 3.42E−02 |
| Actg1 | 26 | 1 | 0.976 | 0.880 | 2.14E−12 | 4.72E−09 |
| Tph1 | 26 | 0.938 | 0.683 | 1.298 | 7.68E−12 | 1.58E−08 |
| Alox5ap | 26 | 0.926 | 0.659 | 1.357 | 5.00E−11 | 9.48E−08 |
| Fcer1a | 26 | 0.983 | 0.976 | 0.700 | 9.83E−10 | 1.55E−06 |
| Mcpt2 | 26 | 0.955 | 0.634 | 1.631 | 1.74E−08 | 2.24E−05 |
| Mcpt1 | 26 | 0.96 | 0.634 | 1.586 | 3.76E−08 | 4.61E−05 |
| Decr1 | 26 | 0.682 | 0.171 | 1.704 | 4.35E−08 | 5.26E−05 |
| Ms4a2 | 26 | 0.932 | 0.707 | 1.066 | 2.04E−07 | 2.18E−04 |
| Itga2b | 26 | 0.818 | 0.293 | 1.452 | 3.40E−07 | 3.45E−04 |
| Ncor1 | 26 | 0.534 | 0.707 | −0.936 | 3.88E−07 | 3.88E−04 |
| Ero1l | 26 | 0.739 | 0.293 | 1.562 | 5.25E−07 | 5.12E−04 |
| Mcpt4 | 26 | 0.835 | 0.488 | 1.895 | 1.56E−06 | 1.39E−03 |
| Sytl3 | 26 | 0.449 | 0.049 | 2.647 | 1.64E−06 | 1.45E−03 |
| Ptpre | 26 | 0.239 | 0.537 | −1.096 | 1.78E−06 | 1.57E−03 |
| Prnp | 26 | 0.92 | 0.805 | 0.856 | 2.01E−06 | 1.76E−03 |
| Fam105a | 26 | 0.784 | 0.293 | 1.562 | 2.65E−06 | 2.28E−03 |
| Gpr171 | 26 | 0.852 | 0.512 | 1.799 | 3.99E−06 | 3.25E−03 |
| Vasp | 26 | 0.892 | 0.537 | 1.081 | 4.68E−06 | 3.75E−03 |
| Scin | 26 | 0.966 | 0.61 | 0.960 | 4.70E−06 | 3.76E−03 |
| Rac2 | 26 | 0.994 | 0.951 | 0.643 | 5.62E−06 | 4.40E−03 |
| Igfbp4 | 26 | 0.045 | 0.244 | −0.802 | 6.08E−06 | 4.71E−03 |
| Pga5 | 26 | 0.307 | 0 | 4.663 | 7.06E−06 | 5.38E−03 |
| Psip1 | 26 | 0.284 | 0.39 | −0.818 | 7.61E−06 | 5.75E−03 |
| Stk19 | 26 | 0.631 | 0.317 | 1.122 | 8.39E−06 | 6.28E−03 |
| Atp1b1 | 26 | 0.92 | 0.659 | 0.831 | 1.53E−05 | 1.07E−02 |
| Gm2a | 26 | 0.938 | 0.854 | 0.723 | 1.63E−05 | 1.13E−02 |
| Opa3 | 26 | 0.193 | 0.22 | −0.937 | 1.68E−05 | 1.16E−02 |
| Ftl1 | 26 | 1 | 1 | 0.846 | 3.43E−05 | 2.21E−02 |
| Il4 | 26 | 0.784 | 0.659 | 1.307 | 5.53E−05 | 3.37E−02 |
| Tacc1 | 26 | 0.415 | 0.122 | 0.633 | 5.83E−05 | 3.53E−02 |
| Ptms | 26 | 0.733 | 0.78 | −0.473 | 6.46E−05 | 3.83E−02 |
| Cox4i1 | 26 | 0.966 | 0.927 | 0.528 | 6.81E−05 | 4.02E−02 |
| Sec11c | 26 | 0.898 | 0.512 | 0.801 | 6.98E−05 | 4.10E−02 |
| Zcchc10 | 26 | 0.46 | 0.122 | 0.773 | 8.08E−05 | 4.66E−02 |
| Mknk2 | 26 | 0.205 | 0.317 | −0.914 | 8.67E−05 | 4.93E−02 |
| Mcpt1 | 27 | 0.177 | 0.017 | 2.416 | 2.33E−05 | 1.57E−02 |
| Kdm6b | 27 | 0.258 | 0.554 | −1.318 | 4.79E−05 | 2.99E−02 |
| Cd84 | 27 | 0.419 | 0.124 | 1.416 | 7.77E−05 | 4.50E−02 |
| Zfp36l2 | 29 | 0.831 | 0.482 | 1.290 | 8.32E−07 | 7.75E−04 |
| Tsc22d3 | 29 | 0.877 | 0.651 | 0.697 | 4.32E−05 | 2.72E−02 |
| P2ry10 | 29 | 0.538 | 0.157 | 1.414 | 4.35E−05 | 2.74E−02 |
| Dstn | 30 | 0.857 | 0.99 | −0.625 | 2.68E−06 | 2.29E−03 |
| Ada | 30 | 0.057 | 0.2 | −4.360 | 5.72E−06 | 4.47E−03 |
| Ubb | 30 | 0.943 | 0.95 | −0.641 | 1.25E−05 | 8.92E−03 |
| Igkc | 30 | 0.743 | 0.52 | 0.553 | 1.25E−05 | 8.92E−03 |
| Apol9a | 30 | 0 | 0.18 | −4.300 | 7.19E−05 | 4.20E−02 |
| Al413582 | 31 | 0.024 | 0.272 | −2.863 | 5.52E−06 | 4.34E−03 |
| Casq2 | 32 | 0.35 | 0.021 | 2.515 | 1.16E−05 | 8.34E−03 |
| Thbs1 | 33 | 0.469 | 0.268 | 1.681 | 6.38E−05 | 3.80E−02 |
| Mcpt2 | 34 | 0.491 | 0.07 | 1.799 | 1.14E−06 | 1.04E−03 |
| Areg | 34 | 0.018 | 0.186 | 1.280 | 2.27E−06 | 1.97E−03 |
| Slc25a5 | 34 | 0.93 | 0.907 | 0.470 | 2.39E−06 | 2.06E−03 |
| Mcpt1 | 34 | 0.474 | 0.07 | 2.144 | 7.59E−06 | 5.74E−03 |
| Runx3 | 34 | 0.702 | 0.977 | −0.675 | 8.83E−06 | 6.57E−03 |
| Defa24 | 34 | 0.175 | 0.558 | −1.480 | 1.20E−05 | 8.62E−03 |
| Zmiz1 | 34 | 0.456 | 0.744 | −0.901 | 1.67E−05 | 1.15E−02 |
| Itm2b | 34 | 0.947 | 0.977 | 0.362 | 3.72E−05 | 2.38E−02 |
| Capns1 | 34 | 0.842 | 0.628 | 0.596 | 6.09E−05 | 3.65E−02 |
| Whrn | 34 | 0 | 0.186 | −3.482 | 8.47E−05 | 4.84E−02 |
| Xrcc6bp1 | 35 | 0 | 0.203 | −3.388 | 1.10E−05 | 8.01E−03 |
| Atf3 | 35 | 0.275 | 0.627 | −0.517 | 4.05E−05 | 2.57E−02 |
| Cxcl2 | 36 | 0.875 | 0.692 | 0.999 | 4.55E−06 | 3.66E−03 |
| Dusp1 | 36 | 0.946 | 0.821 | 0.719 | 5.85E−06 | 4.56E−03 |
| S100a8 | 36 | 0.982 | 0.795 | 1.265 | 7.62E−06 | 5.75E−03 |
| Actg1 | 36 | 0.964 | 1 | 0.587 | 2.04E−05 | 1.38E−02 |
| S100a9 | 36 | 0.946 | 0.923 | 1.043 | 3.43E−05 | 2.21E−02 |
| G0s2 | 36 | 0.893 | 0.846 | 0.800 | 7.82E−05 | 4.52E−02 |
| Il1b | 36 | 0.982 | 1 | 0.474 | 8.19E−05 | 4.70E−02 |
| Ptgs2 | 36 | 0.661 | 0.282 | 1.505 | 8.59E−05 | 4.90E−02 |
| Hmgcs1 | 37 | 0 | 0.306 | −4.058 | 5.86E−05 | 3.54E−02 |
| Mcpt1 | 41 | 0.458 | 0 | 4.593 | 1.64E−05 | 1.14E−02 |
| Alcam | 41 | 0.583 | 0.04 | 2.248 | 1.93E−05 | 1.32E−02 |
| Ptp4a3 | 41 | 0.625 | 1 | −0.853 | 4.79E−05 | 2.99E−02 |
| Acbd5 | 41 | 0.667 | 0.12 | 1.926 | 8.37E−05 | 4.80E−02 |
| Itgal | 42 | 0.375 | 0.125 | −0.238 | 1.65E−07 | 1.80E−04 |
| Reep5 | 43 | 0.786 | 0.5 | 0.884 | 6.46E−06 | 4.98E−03 |
| Tceb2 | 44 | 0.588 | 0 | 6.153 | 2.88E−05 | 1.89E−02 |

B

| | | percentage of | percentage of | | | |
|---|---|---|---|---|---|---|
| | | expressing | expressing | | | |
| | cluster | cells in OVA- | cells in PBS- | LN (average | | p value, |
| gene | ID | treated group | treated group | fold change) | p value | adjusted |
| Actb | 1 | 1 | 0.968 | 1.320 | 1.82E−23 | 5.92E−20 |
| Ighg1 | 1 | 0.372 | 0.008 | 3.863 | 2.30E−14 | 3.41E−11 |
| Ptprcap | 1 | 0.653 | 0.376 | 0.960 | 2.96E−09 | 2.23E−06 |
| Igha | 1 | 0.992 | 1 | 0.613 | 1.87E−08 | 1.23E−05 |
| Pfn1 | 1 | 1 | 0.944 | 0.431 | 1.51E−07 | 8.39E−05 |
| Emb | 1 | 0.545 | 0.816 | −0.648 | 4.11E−07 | 2.09E−04 |
| Igkc | 1 | 1 | 1 | 0.926 | 4.21E−07 | 2.13E−04 |
| Ddit4 | 1 | 0.157 | 0 | 3.923 | 4.72E−07 | 2.36E−04 |
| Mcpt1 | 1 | 0.157 | 0.008 | 2.804 | 1.94E−05 | 6.35E−03 |
| Maff | 1 | 0.107 | 0.32 | −0.879 | 2.13E−05 | 6.90E−03 |
| Cfl1 | 1 | 0.934 | 0.912 | 0.344 | 3.23E−05 | 9.88E−03 |
| Rras2 | 1 | 0.099 | 0.296 | −1.281 | 3.71E−05 | 1.12E−02 |
| Dusp10 | 1 | 0.612 | 0.768 | −0.557 | 4.19E−05 | 1.24E−02 |
| Cnn2 | 1 | 0.678 | 0.424 | 0.641 | 4.42E−05 | 1.30E−02 |
| Jun | 1 | 0.438 | 0.232 | 1.206 | 4.47E−05 | 1.31E−02 |
| Myl6 | 1 | 0.909 | 0.808 | 0.369 | 7.44E−05 | 2.03E−02 |
| Ppp1r16b | 1 | 0.281 | 0.072 | 1.323 | 7.72E−05 | 2.10E−02 |
| Dusp5 | 1 | 0.289 | 0.552 | −0.744 | 7.95E−05 | 2.15E−02 |
| Igkv1-135 | 1 | 0.033 | 0.2 | −1.720 | 9.18E−05 | 2.42E−02 |
| Tmem71 | 1 | 0.066 | 0.264 | −1.482 | 9.45E−05 | 2.48E−02 |
| Rgs1 | 1 | 0.306 | 0.096 | 1.424 | 1.01E−04 | 2.62E−02 |
| Hsp90ab1 | 1 | 0.76 | 0.624 | 0.546 | 1.07E−04 | 2.76E−02 |
| Fam169b | 1 | 0.033 | 0.184 | −1.553 | 1.14E−04 | 2.91E−02 |
| Cd52 | 1 | 0.893 | 0.816 | 0.412 | 1.17E−04 | 2.98E−02 |
| Cct8 | 1 | 0.347 | 0.12 | 1.026 | 1.96E−04 | 4.59E−02 |
| Mcpt1 | 11 | 0.167 | 0 | 4.314 | 3.70E−09 | 2.72E−06 |
| Ikzf2 | 11 | 0.369 | 0.724 | −0.930 | 5.16E−07 | 2.57E−04 |
| Rgs1 | 11 | 0.821 | 0.704 | 0.745 | 1.31E−06 | 5.92E−04 |
| Dusp2 | 11 | 0.464 | 0.24 | 1.175 | 2.06E−06 | 8.78E−04 |
| Igha | 11 | 0.988 | 0.913 | 0.278 | 2.41E−06 | 1.01E−03 |
| Def6 | 11 | 0.357 | 0.112 | 1.164 | 1.66E−05 | 5.56E−03 |
| Bhlhe40 | 11 | 0.429 | 0.73 | −0.539 | 4.53E−05 | 1.33E−02 |
| Top1 | 11 | 0.143 | 0.321 | −0.475 | 4.63E−05 | 1.35E−02 |
| Zfp36l2 | 11 | 0.774 | 0.663 | 0.442 | 6.65E−05 | 1.85E−02 |
| Pdcd4 | 11 | 0.393 | 0.423 | 0.429 | 7.20E−05 | 1.97E−02 |
| Rbm5 | 11 | 0.036 | 0.214 | −2.020 | 7.61E−05 | 2.08E−02 |
| Pim1 | 11 | 0.536 | 0.796 | −0.640 | 8.10E−05 | 2.18E−02 |
| Neat1 | 11 | 0.321 | 0.388 | 0.273 | 8.63E−05 | 2.30E−02 |
| Napsa | 11 | 0.25 | 0.092 | 0.833 | 1.74E−04 | 4.15E−02 |
| Bcl2 | 11 | 0.821 | 0.755 | 0.439 | 2.11E−04 | 4.89E−02 |
| Srgn | 12 | 0.935 | 0.714 | 0.951 | 2.14E−85 | 1.55E−80 |
| Hes1 | 12 | 0.487 | 0.871 | −1.225 | 1.02E−69 | 4.26E−65 |
| Sla | 12 | 0.6 | 0.182 | 1.644 | 6.69E−66 | 2.59E−61 |
| Ppp1r15a | 12 | 0.4 | 0.741 | −1.371 | 3.00E−64 | 1.03E−59 |
| Furin | 12 | 0.727 | 0.359 | 1.650 | 2.64E−63 | 7.31E−59 |
| Actg1 | 12 | 0.926 | 0.77 | 0.782 | 4.58E−63 | 1.21E−58 |
| Il13 | 12 | 0.445 | 0.093 | 2.748 | 4.80E−55 | 8.74E−51 |
| Hspa8 | 12 | 0.929 | 0.783 | 0.644 | 3.71E−49 | 5.26E−45 |
| Lilr4b | 12 | 0.345 | 0.038 | 2.439 | 6.08 E−48 | 7.86E−44 |
| 4930523C07Rik | 12 | 0.433 | 0.747 | −0.954 | 5.86E−46 | 6.69E−42 |
| Dnaja1 | 12 | 0.694 | 0.847 | −0.898 | 5.15E−45 | 5.55E−41 |
| Vps37b | 12 | 0.568 | 0.811 | −0.893 | 3.69E−43 | 3.64E−39 |
| Nr4a3 | 12 | 0.524 | 0.169 | 1.521 | 2.56E−42 | 2.48E−38 |
| Plaur | 12 | 0.374 | 0.078 | 2.052 | 2.02E−40 | 1.78E−36 |
| Fos | 12 | 0.572 | 0.82 | −1.132 | 1.82E−39 | 1.56E−35 |
| Lif | 12 | 0.263 | 0.022 | 2.855 | 5.25E−39 | 4.37E−35 |
| Zfp36l1 | 12 | 0.938 | 0.82 | 0.631 | 6.03E−39 | 4.94E−35 |
| Cxcr6 | 12 | 0.625 | 0.301 | 1.109 | 1.02E−37 | 7.81E−34 |
| Sptssa | 12 | 0.501 | 0.769 | −0.841 | 8.77E−36 | 6.38E−32 |
| Dusp1 | 12 | 0.62 | 0.867 | −0.896 | 2.80E−33 | 1.79E−29 |
| Ubb | 12 | 0.919 | 0.965 | −0.413 | 1.48E−32 | 8.78E−29 |
| Plk3 | 12 | 0.285 | 0.051 | 1.772 | 8.17E−31 | 4.40E−27 |
| Areg | 12 | 0.498 | 0.224 | 1.394 | 2.69E−30 | 1.40E−26 |
| Nab2 | 12 | 0.182 | 0.007 | 2.979 | 6.12E−30 | 3.12E−26 |
| Pdcd1 | 12 | 0.244 | 0.033 | 2.125 | 1.16E−29 | 5.83E−26 |
| Il4 | 12 | 0.519 | 0.301 | 1.409 | 2.35E−28 | 1.14E−24 |
| Gcnt1 | 12 | 0.217 | 0.024 | 2.384 | 4.66E−28 | 2.19E−24 |
| Jund | 12 | 0.864 | 0.964 | −0.512 | 5.58E−27 | 2.42E−23 |
| Fosl2 | 12 | 0.614 | 0.332 | 0.801 | 3.42E−26 | 1.41E−22 |
| H2-D1 | 12 | 0.972 | 0.995 | −0.326 | 1.13E−25 | 4.46E−22 |
| Tpt1 | 12 | 0.985 | 0.976 | 0.333 | 1.22E−25 | 4.76E−22 |
| Prr7 | 12 | 0.09 | 0.281 | −1.547 | 5.86E−25 | 2.22E−21 |
| Sh2d2a | 12 | 0.435 | 0.186 | 0.936 | 1.07E−24 | 3.90E−21 |
| Esm1 | 12 | 0.018 | 0.173 | −2.314 | 3.73E−24 | 1.29E−20 |
| Crem | 12 | 0.717 | 0.443 | 0.579 | 5.47E−24 | 1.88E−20 |
| Fosb | 12 | 0.412 | 0.616 | −0.805 | 9.76E−24 | 3.28E−20 |
| Rap1b | 12 | 0.663 | 0.444 | 0.613 | 2.09E−23 | 6.74E−20 |
| Csf2 | 12 | 0.258 | 0.067 | 1.960 | 2.13E−23 | 6.81E−20 |
| Ptpn22 | 12 | 0.313 | 0.095 | 1.251 | 3.56E−23 | 1.12E−19 |
| Lilrb4a | 12 | 0.206 | 0.029 | 2.154 | 3.70E−23 | 1.16E−19 |
| Lmo4 | 12 | 0.914 | 0.974 | −0.391 | 1.10E−22 | 3.37E−19 |
| Cdk11b | 12 | 0.133 | 0.306 | −1.282 | 2.16E−22 | 6.48E−19 |
| Lgals7 | 12 | 0.291 | 0.08 | 1.511 | 2.32E−22 | 6.93E−19 |
| Nfkbiz | 12 | 0.729 | 0.525 | 0.624 | 3.17E−20 | 8.28E−17 |
| Actb | 12 | 0.964 | 0.903 | 0.686 | 1.26E−19 | 3.07E−16 |
| Zap70 | 12 | 0.424 | 0.2 | 0.882 | 1.26E−19 | 3.07E−16 |
| Fam46a | 12 | 0.425 | 0.211 | 1.080 | 5.68E−19 | 1.32E−15 |
| Dusp10 | 12 | 0.273 | 0.448 | −0.820 | 5.80E−19 | 1.34E−15 |
| Spry2 | 12 | 0.427 | 0.204 | 1.201 | 7.86E−19 | 1.79E−15 |
| Cytip | 12 | 0.625 | 0.392 | 0.531 | 1.50E−18 | 3.34E−15 |
| Siah2 | 12 | 0.082 | 0.253 | −1.422 | 1.68E−18 | 3.71E−15 |
| Wnk1 | 12 | 0.398 | 0.197 | 0.971 | 2.95E−18 | 6.31E−15 |
| Dtx1 | 12 | 0.05 | 0.197 | −1.600 | 4.37E−18 | 9.28E−15 |
| Tnfaip8 | 12 | 0.518 | 0.299 | 0.740 | 6.98E−18 | 1.45E−14 |
| Fgl2 | 12 | 0.61 | 0.388 | 0.609 | 1.62E−17 | 3.21E−14 |
| Prdx6 | 12 | 0.507 | 0.33 | 0.908 | 2.87E−17 | 5.57E−14 |
| Tpm4 | 12 | 0.394 | 0.193 | 0.964 | 2.87E−17 | 5.57E−14 |
| Serp1 | 12 | 0.554 | 0.692 | −0.529 | 3.88E−17 | 7.46E−14 |
| Ero1l | 12 | 0.399 | 0.213 | 0.949 | 5.36E−17 | 1.01E−13 |
| Uqcrh | 12 | 0.765 | 0.596 | 0.450 | 6.48E−17 | 1.21E−13 |
| Arl5c | 12 | 0.04 | 0.173 | −1.873 | 7.55E−17 | 1.39E−13 |
| Coq10b | 12 | 0.332 | 0.521 | −0.690 | 9.27E−17 | 1.69E−13 |
| Pfn1 | 12 | 0.974 | 0.962 | 0.233 | 9.91E−17 | 1.80E−13 |
| Sepp1 | 12 | 0.254 | 0.439 | −0.824 | 1.47E−16 | 2.64E−13 |
| Ucp2 | 12 | 0.612 | 0.43 | 0.519 | 4.62E−16 | 8.09E−13 |
| Pnrc1 | 12 | 0.583 | 0.774 | −0.457 | 5.82E−16 | 1.00E−12 |
| Gpr132 | 12 | 0.399 | 0.539 | −0.573 | 1.25E−15 | 2.09E−12 |
| Tiparp | 12 | 0.18 | 0.33 | −0.904 | 1.88E−15 | 3.10E−12 |
| Hccs | 12 | 0.243 | 0.08 | 1.079 | 3.32E−15 | 5.37E−12 |
| Eef1a1 | 12 | 0.997 | 0.995 | 0.236 | 4.21E−15 | 6.72E−12 |
| Lmna | 12 | 0.045 | 0.173 | −1.627 | 4.61E−15 | 7.30E−12 |
| Cd52 | 12 | 0.581 | 0.397 | 0.644 | 4.69E−15 | 7.38E−12 |
| Cycs | 12 | 0.531 | 0.337 | 0.561 | 4.96E−15 | 7.79E−12 |
| Igha | 12 | 0.951 | 0.907 | 0.407 | 6.51E−15 | 1.02E−11 |
| Rheb | 12 | 0.536 | 0.342 | 0.631 | 1.00E−14 | 1.54E−11 |
| Igkc | 12 | 0.985 | 0.974 | 0.474 | 1.09E−14 | 1.68E−11 |
| Kif23 | 12 | 0.102 | 0.259 | −1.129 | 1.13E−14 | 1.71E−11 |
| Cited2 | 12 | 0.176 | 0.375 | −0.724 | 1.18E−14 | 1.79E−11 |
| Klf4 | 12 | 0.336 | 0.537 | −0.566 | 3.70E−14 | 5.32E−11 |
| Nfkb1 | 12 | 0.81 | 0.674 | 0.501 | 4.40E−14 | 6.26E−11 |
| BC031181 | 12 | 0.365 | 0.454 | −0.512 | 5.80E−14 | 8.17E−11 |
| Ramp3 | 12 | 0.561 | 0.342 | 0.516 | 7.44E−14 | 1.03E−10 |
| Cwc25 | 12 | 0.061 | 0.188 | −1.439 | 9.00E−14 | 1.24E−10 |
| Scp2 | 12 | 0.382 | 0.559 | −0.568 | 9.01E−14 | 1.24E−10 |
| Eif3e | 12 | 0.67 | 0.512 | 0.416 | 2.54E−13 | 3.33E−10 |
| Tnfaip3 | 12 | 0.534 | 0.643 | −0.487 | 2.72E−13 | 3.55E−10 |
| Irf1 | 12 | 0.069 | 0.206 | −1.336 | 2.81E−13 | 3.66E−10 |
| Nrgn | 12 | 0.284 | 0.12 | 1.042 | 3.57E−13 | 4.57E−10 |
| Abhd17c | 12 | 0.52 | 0.324 | 0.599 | 5.00E−13 | 6.29E−10 |
| Glul | 12 | 0.184 | 0.339 | −1.009 | 6.84E−13 | 8.52E−10 |
| Vmp1 | 12 | 0.239 | 0.093 | 1.019 | 8.14E−13 | 1.01E−09 |
| Gmfg | 12 | 0.31 | 0.142 | 0.821 | 1.34E−12 | 1.62E−09 |
| Tmem64 | 12 | 0.41 | 0.224 | 0.605 | 1.76E−12 | 2.11E−09 |
| Rnf19b | 12 | 0.279 | 0.118 | 0.971 | 3.11E−12 | 3.58E−09 |
| Naca | 12 | 0.839 | 0.723 | 0.299 | 4.17E−12 | 4.76E−09 |
| Gimap5 | 12 | 0.338 | 0.179 | 0.779 | 8.34E−12 | 9.16E−09 |
| Arf6 | 12 | 0.429 | 0.268 | 0.610 | 9.31E−12 | 1.01E−08 |
| Tmsb4x | 12 | 1 | 1 | 0.195 | 9.66E−12 | 1.05E−08 |
| Eef1b2 | 12 | 0.709 | 0.554 | 0.395 | 1.30E−11 | 1.39E−08 |
| Eif1 | 12 | 0.996 | 0.989 | 0.152 | 1.86E−11 | 1.96E−08 |
| Arpc2 | 12 | 0.769 | 0.654 | 0.318 | 2.02E−11 | 2.12E−08 |
| H2-Q4 | 12 | 0.246 | 0.41 | −0.675 | 2.07E−11 | 2.17E−08 |
| Gpx4 | 12 | 0.818 | 0.887 | −0.345 | 2.52E−11 | 2.63E−08 |
| Emd | 12 | 0.235 | 0.353 | −0.729 | 2.83E−11 | 2.94E−08 |
| Polr2e | 12 | 0.091 | 0.226 | −1.019 | 4.42E−11 | 4.49 E−08 |
| Tspan13 | 12 | 0.699 | 0.787 | −0.366 | 4.65E−11 | 4.69E−08 |
| Zc3h12a | 12 | 0.157 | 0.301 | −0.866 | 8.60E−11 | 8.39E−08 |
| Itk | 12 | 0.515 | 0.332 | 0.495 | 8.94E−11 | 8.69E−08 |
| Maff | 12 | 0.399 | 0.519 | −0.518 | 9.49E−11 | 9.18E−08 |
| Rsrp1 | 12 | 0.24 | 0.386 | −0.708 | 1.16E−10 | 1.11E−07 |
| Tgfb1 | 12 | 0.457 | 0.293 | 0.625 | 1.48E−10 | 1.39E−07 |
| Ptp4a1 | 12 | 0.349 | 0.466 | −0.514 | 1.88E−10 | 1.74E−07 |
| Gapdh | 12 | 0.561 | 0.434 | 0.420 | 2.03E−10 | 1.87E−07 |
| Ly6a | 12 | 0.055 | 0.173 | −1.208 | 2.28E−10 | 2.09E−07 |
| Iglc1 | 12 | 0.184 | 0.311 | −0.692 | 2.55E−10 | 2.32E−07 |
| Psap | 12 | 0.299 | 0.437 | −0.562 | 3.21E−10 | 2.88E−07 |
| Pole4 | 12 | 0.604 | 0.457 | 0.415 | 3.83E−10 | 3.35E−07 |
| Cox8a | 12 | 0.798 | 0.71 | 0.285 | 3.88E−10 | 3.38E−07 |
| Ndfip1 | 12 | 0.247 | 0.109 | 0.765 | 4.09E−10 | 3.56E−07 |
| Eef2 | 12 | 0.927 | 0.898 | 0.240 | 5.72E−10 | 4.91E−07 |
| Tgoln1 | 12 | 0.215 | 0.326 | −0.572 | 5.85E−10 | 5.00E−07 |
| Jarid2 | 12 | 0.156 | 0.046 | 1.269 | 6.36E−10 | 5.40E−07 |
| Galnt12 | 12 | 0.086 | 0.209 | −0.960 | 7.06E−10 | 5.97E−07 |
| Ddx5 | 12 | 0.886 | 0.931 | −0.276 | 7.94E−10 | 6.71E−07 |
| Kdm6b | 12 | 0.65 | 0.47 | 0.555 | 9.04E−10 | 7.58E−07 |
| Pgm2l1 | 12 | 0.065 | 0.177 | −0.966 | 1.03E−09 | 8.51E−07 |
| Tspan32 | 12 | 0.073 | 0.184 | −0.997 | 1.15E−09 | 9.39E−07 |
| Zfp36l2 | 12 | 0.49 | 0.448 | 0.453 | 1.17E−09 | 9.52E−07 |
| Fth1 | 12 | 0.963 | 0.989 | −0.236 | 1.23E−09 | 1.00E−06 |
| Ubc | 12 | 0.303 | 0.446 | −0.542 | 1.29E−09 | 1.04E−06 |
| Mat2a | 12 | 0.288 | 0.415 | −0.572 | 1.34E−09 | 1.08E−06 |
| Nxpe3 | 12 | 0.202 | 0.082 | 0.974 | 1.64E−09 | 1.30E−06 |
| Cbarp | 12 | 0.193 | 0.077 | 0.886 | 1.74E−09 | 1.38E−06 |
| Nr4a2 | 12 | 0.383 | 0.259 | 0.702 | 2.50E−09 | 1.92E−06 |
| H3f3b | 12 | 0.982 | 0.982 | 0.286 | 2.59E−09 | 1.98E−06 |
| Fxyd5 | 12 | 0.564 | 0.432 | 0.407 | 2.82E−09 | 2.14E−06 |
| Neurl3 | 12 | 0.328 | 0.439 | −0.470 | 3.18E−09 | 2.38E−06 |
| Arl4a | 12 | 0.256 | 0.128 | 0.781 | 3.23E−09 | 2.40E−06 |
| Psmb10 | 12 | 0.219 | 0.335 | −0.598 | 3.50E−09 | 2.60E−06 |
| Cntnap2 | 12 | 0.074 | 0.175 | −1.008 | 4.85E−09 | 3.50E−06 |
| Usmg5 | 12 | 0.509 | 0.585 | −0.414 | 5.00E−09 | 3.60E−06 |
| Zrsr1 | 12 | 0.242 | 0.337 | −0.652 | 5.12E−09 | 3.68E−06 |
| H2afj | 12 | 0.433 | 0.29 | 0.452 | 7.92E−09 | 5.53E−06 |
| Ccrl2 | 12 | 0.192 | 0.308 | −0.742 | 8.29E−09 | 5.78E−06 |
| Psmb8 | 12 | 0.49 | 0.627 | −0.441 | 8.39E−09 | 5.84E−06 |
| Tmsb10 | 12 | 0.942 | 0.976 | −0.240 | 1.08E−08 | 7.39E−06 |
| Ccl1 | 12 | 0.203 | 0.084 | 1.335 | 1.17E−08 | 7.93E−06 |
| Saraf | 12 | 0.304 | 0.435 | −0.533 | 1.19E−08 | 8.02E−06 |
| Uhrf2 | 12 | 0.296 | 0.424 | −0.506 | 1.19E−08 | 8.02E−06 |
| Eif3h | 12 | 0.686 | 0.568 | 0.298 | 1.64E−08 | 1.09E−05 |
| Srsf7 | 12 | 0.251 | 0.311 | −0.443 | 2.24E−08 | 1.45E−05 |
| Hmgb1 | 12 | 0.552 | 0.687 | −0.400 | 2.28E−08 | 1.47E−05 |
| H2-K1 | 12 | 0.921 | 0.958 | −0.243 | 2.81E−08 | 1.79E−05 |
| Gypc | 12 | 0.256 | 0.133 | 0.686 | 2.89E−08 | 1.84E−05 |
| Dbp | 12 | 0.059 | 0.151 | −1.018 | 3.11E−08 | 1.96E−05 |
| Lcp2 | 12 | 0.339 | 0.208 | 0.544 | 3.75E−08 | 2.32E−05 |
| Pnn | 12 | 0.123 | 0.235 | −0.769 | 3.80E−08 | 2.35E−05 |
| Nab1 | 12 | 0.193 | 0.082 | 0.888 | 3.94E−08 | 2.42E−05 |
| 2810474O19Rik | 12 | 0.099 | 0.195 | −0.835 | 4.39E−08 | 2.67E−05 |
| Hist1h1c | 12 | 0.205 | 0.102 | 1.035 | 4.46E−08 | 2.71E−05 |
| Pdcd1lg2 | 12 | 0.223 | 0.109 | 0.833 | 4.95E−08 | 2.98E−05 |
| Tmem59 | 12 | 0.332 | 0.463 | −0.485 | 5.65E−08 | 3.37E−05 |
| Tapbp | 12 | 0.287 | 0.403 | −0.510 | 5.78E−08 | 3.44E−05 |
| Tob2 | 12 | 0.328 | 0.434 | −0.479 | 6.05E−08 | 3.57E−05 |
| Hmha1 | 12 | 0.206 | 0.333 | −0.645 | 6.95E−08 | 4.06E−05 |
| Slc3a2 | 12 | 0.47 | 0.321 | 0.469 | 7.95E−08 | 4.59E−05 |
| Fam189b | 12 | 0.116 | 0.226 | −0.759 | 9.57E−08 | 5.44E−05 |
| Churc1 | 12 | 0.258 | 0.357 | −0.570 | 1.08E−07 | 6.12E−05 |
| Clk1 | 12 | 0.269 | 0.362 | −0.438 | 1.26E−07 | 7.07E−05 |
| Cox4i1 | 12 | 0.836 | 0.765 | 0.208 | 1.70E−07 | 9.31E−05 |
| Tprgl | 12 | 0.291 | 0.401 | −0.513 | 1.76E−07 | 9.63E−05 |
| Rnf167 | 12 | 0.098 | 0.2 | −0.794 | 1.93E−07 | 1.05E−04 |
| Eprs | 12 | 0.133 | 0.231 | −0.676 | 2.01E−07 | 1.09E−04 |
| Polr2j | 12 | 0.099 | 0.206 | −0.824 | 2.02E−07 | 1.09E−04 |
| Slc25a5 | 12 | 0.458 | 0.352 | 0.368 | 2.12E−07 | 1.14E−04 |
| Ak2 | 12 | 0.288 | 0.169 | 0.603 | 2.14E−07 | 1.15E−04 |
| Cd53 | 12 | 0.687 | 0.566 | 0.260 | 2.45E−07 | 1.30E−04 |
| Jun | 12 | 0.456 | 0.534 | −0.614 | 2.67E−07 | 1.41E−04 |
| Nr3c1 | 12 | 0.487 | 0.346 | 0.471 | 2.92E−07 | 1.53E−04 |
| Srrm2 | 12 | 0.433 | 0.455 | −0.241 | 3.14E−07 | 1.63E−04 |
| Selk | 12 | 0.749 | 0.661 | 0.258 | 3.40E−07 | 1.75E−04 |
| Cotl1 | 12 | 0.481 | 0.61 | −0.396 | 3.52E−07 | 1.80E−04 |
| Hsp90ab1 | 12 | 0.585 | 0.485 | 0.369 | 3.91E−07 | 1.99E−04 |
| Bcl11b | 12 | 0.115 | 0.215 | −0.742 | 4.36E−07 | 2.20E−04 |
| Taldo1 | 12 | 0.399 | 0.508 | −0.406 | 4.61E−07 | 2.31E−04 |
| Orai1 | 12 | 0.247 | 0.129 | 0.719 | 4.95E−07 | 2.47E−04 |
| Pfdn5 | 12 | 0.778 | 0.69 | 0.229 | 6.11E−07 | 2.99E−04 |
| Rxrg | 12 | 0.089 | 0.191 | −0.813 | 6.16E−07 | 3.01E−04 |
| Maf1 | 12 | 0.122 | 0.209 | −0.730 | 6.66E−07 | 3.23E−04 |
| Spcs2 | 12 | 0.583 | 0.709 | −0.346 | 7.47E−07 | 3.56E−04 |
| Ralbp1 | 12 | 0.26 | 0.362 | −0.471 | 7.89E−07 | 3.75E−04 |
| Hlf | 12 | 0.25 | 0.135 | 0.620 | 8.37E−07 | 3.95E−04 |
| Smco4 | 12 | 0.185 | 0.089 | 1.106 | 9.51E−07 | 4.44E−04 |
| Fam102a | 12 | 0.31 | 0.191 | 0.548 | 1.08E−06 | 5.00E−04 |
| Btg2 | 12 | 0.945 | 0.9 | 0.203 | 1.08E−06 | 5.01E−04 |
| Rab26os | 12 | 0.094 | 0.175 | −0.713 | 1.09E−06 | 5.02E−04 |
| Runx3 | 12 | 0.194 | 0.31 | −0.586 | 1.27E−06 | 5.77E−04 |
| Cd47 | 12 | 0.312 | 0.393 | −0.415 | 1.28E−06 | 5.82E−04 |
| Fam32a | 12 | 0.169 | 0.255 | −0.572 | 1.33E−06 | 5.97E−04 |
| Dusp5 | 12 | 0.563 | 0.43 | 0.426 | 1.53E−06 | 6.77E−04 |
| Rgs2 | 12 | 0.832 | 0.796 | 0.360 | 1.61E−06 | 7.11E−04 |
| Lgals9 | 12 | 0.156 | 0.244 | −0.599 | 1.68E−06 | 7.36E−04 |
| Sec11c | 12 | 0.403 | 0.51 | −0.377 | 1.84E−06 | 7.99E−04 |
| Zyx | 12 | 0.188 | 0.093 | 0.712 | 1.85E−06 | 8.02E−04 |
| Eif4a1 | 12 | 0.671 | 0.585 | 0.213 | 2.01E−06 | 8.59E−04 |
| Nkg7 | 12 | 0.445 | 0.57 | −0.379 | 2.34E−06 | 9.88E−04 |
| 1500011K16Rik | 12 | 0.135 | 0.157 | −0.378 | 2.46E−06 | 1.03E−03 |
| Rsbn1l | 12 | 0.157 | 0.2 | −0.441 | 2.47E−06 | 1.03E−03 |
| Hmgn2 | 12 | 0.091 | 0.182 | −0.797 | 2.67E−06 | 1.10E−03 |
| Npm1 | 12 | 0.704 | 0.623 | 0.255 | 2.73E−06 | 1.13E−03 |
| Srsf3 | 12 | 0.35 | 0.468 | −0.403 | 2.77E−06 | 1.14E−03 |
| Arpc5 | 12 | 0.507 | 0.386 | 0.280 | 2.97E−06 | 1.21E−03 |
| Cox7a2 | 12 | 0.621 | 0.51 | 0.217 | 2.98E−06 | 1.21E−03 |
| Cmtm6 | 12 | 0.099 | 0.195 | −0.711 | 3.16E−06 | 1.27E−03 |
| Asb2 | 12 | 0.219 | 0.131 | 0.449 | 3.36E−06 | 1.35E−03 |
| Rap1a | 12 | 0.338 | 0.226 | 0.514 | 3.50E−06 | 1.39E−03 |
| Map1lc3a | 12 | 0.217 | 0.284 | −0.448 | 3.60E−06 | 1.43E−03 |
| Ier2 | 12 | 0.663 | 0.747 | −0.498 | 3.87E−06 | 1.52E−03 |
| Eif3m | 12 | 0.406 | 0.29 | 0.403 | 3.88E−06 | 1.52E−03 |
| Hspa4 | 12 | 0.247 | 0.142 | 0.563 | 4.09E−06 | 1.60E−03 |
| Neat1 | 12 | 0.329 | 0.22 | 0.571 | 4.14E−06 | 1.62E−03 |
| 1110004F10Rik | 12 | 0.148 | 0.233 | −0.584 | 4.25E−06 | 1.66E−03 |
| Ikzf2 | 12 | 0.181 | 0.237 | −0.586 | 4.85E−06 | 1.87E−03 |
| Polr2m | 12 | 0.214 | 0.308 | −0.492 | 5.09E−06 | 1.96E−03 |
| Itpkb | 12 | 0.277 | 0.189 | 0.222 | 5.10E−06 | 1.96E−03 |
| Tubb4b | 12 | 0.086 | 0.157 | −0.768 | 5.16E−06 | 1.98E−03 |
| Tbck | 12 | 0.153 | 0.239 | −0.562 | 5.24E−06 | 2.01E−03 |
| Vrk1 | 12 | 0.144 | 0.233 | −0.650 | 5.58E−06 | 2.12E−03 |
| Ago2 | 12 | 0.207 | 0.107 | 0.728 | 5.85E−06 | 2.21E−03 |
| Tsc22d3 | 12 | 0.255 | 0.357 | −0.557 | 5.98E−06 | 2.25E−03 |
| Ppp1r16b | 12 | 0.279 | 0.162 | 0.621 | 6.17E−06 | 2.32E−03 |
| Dhx40 | 12 | 0.184 | 0.086 | 0.750 | 6.25E−06 | 2.35E−03 |
| Cmc2 | 12 | 0.203 | 0.255 | −0.410 | 6.86E−06 | 2.55E−03 |
| Lcp1 | 12 | 0.314 | 0.206 | 0.393 | 6.99E−06 | 2.59E−03 |
| Gnb2l1 | 12 | 0.76 | 0.701 | 0.257 | 7.40E−06 | 2.72E−03 |
| Taf7 | 12 | 0.09 | 0.153 | −0.639 | 7.50E−06 | 2.76E−03 |
| Faah | 12 | 0.178 | 0.288 | −0.572 | 7.99E−06 | 2.91E−03 |
| Trappc6b | 12 | 0.267 | 0.273 | −0.211 | 8.10E−06 | 2.95E−03 |
| Nsa2 | 12 | 0.299 | 0.191 | 0.379 | 8.74E−06 | 3.16E−03 |
| Ip6k1 | 12 | 0.083 | 0.157 | −0.767 | 8.97E−06 | 3.23E−03 |
| Tnfrsf1b | 12 | 0.16 | 0.073 | 0.769 | 8.99E−06 | 3.23E−03 |
| Sh3bgrl3 | 12 | 0.754 | 0.803 | −0.224 | 9.42E−06 | 3.37E−03 |
| Abhd17b | 12 | 0.091 | 0.173 | −0.752 | 9.44E−06 | 3.38E−03 |
| Calm3 | 12 | 0.329 | 0.432 | −0.423 | 9.69E−06 | 3.46E−03 |
| Hsp90aa1 | 12 | 0.387 | 0.299 | 0.543 | 9.73E−06 | 3.48E−03 |
| Ddx3x | 12 | 0.433 | 0.308 | 0.376 | 1.06E−05 | 3.75E−03 |
| Ramp1 | 12 | 0.148 | 0.244 | −0.600 | 1.13E−05 | 3.96E−03 |
| Ndel1 | 12 | 0.09 | 0.158 | −0.699 | 1.15E−05 | 4.02E−03 |
| Pebp1 | 12 | 0.255 | 0.342 | −0.433 | 1.19E−05 | 4.13E−03 |
| Tonsl | 12 | 0.402 | 0.472 | −0.339 | 1.30E−05 | 4.49E−03 |
| Kcnn4 | 12 | 0.111 | 0.2 | −0.711 | 1.35E−05 | 4.66E−03 |
| Cldnd1 | 12 | 0.238 | 0.144 | 0.683 | 1.37E−05 | 4.70E−03 |
| Ssbp2 | 12 | 0.106 | 0.164 | −0.559 | 1.61E−05 | 5.43E−03 |
| Arl2bp | 12 | 0.14 | 0.222 | −0.567 | 1.63E−05 | 5.48E−03 |
| Tgm2 | 12 | 0.116 | 0.188 | −0.651 | 1.68E−05 | 5.61E−03 |
| Lsp1 | 12 | 0.285 | 0.353 | −0.396 | 1.88E−05 | 6.19E−03 |
| Rbm5 | 12 | 0.11 | 0.171 | −0.602 | 1.91E−05 | 6.27E−03 |
| Glrx | 12 | 0.314 | 0.222 | 0.253 | 1.93E−05 | 6.33E−03 |
| Tnfrsf18 | 12 | 0.523 | 0.541 | −0.238 | 1.97E−05 | 6.44E−03 |
| Arih1 | 12 | 0.136 | 0.171 | −0.537 | 2.05E−05 | 6.70E−03 |
| Dad1 | 12 | 0.495 | 0.501 | −0.321 | 2.28E−05 | 7.31E−03 |
| Hmgb2 | 12 | 0.382 | 0.488 | −0.362 | 2.42E−05 | 7.71E−03 |
| Zcchc10 | 12 | 0.213 | 0.124 | 0.523 | 2.66E−05 | 8.33E−03 |
| Mkrn1 | 12 | 0.231 | 0.242 | −0.228 | 2.69E−05 | 8.40E−03 |
| Slc6a13 | 12 | 0.122 | 0.209 | −0.662 | 2.89E−05 | 8.96E−03 |
| Gpsm3 | 12 | 0.413 | 0.521 | −0.352 | 2.91E−05 | 9.01E−03 |
| Tmem30a | 12 | 0.326 | 0.222 | 0.395 | 3.02E−05 | 9.33E−03 |
| Klf3 | 12 | 0.147 | 0.239 | −0.583 | 3.06E−05 | 9.43E−03 |
| Cd7 | 12 | 0.075 | 0.155 | −0.714 | 3.07E−05 | 9.44E−03 |
| Eif3f | 12 | 0.769 | 0.699 | 0.189 | 3.24E−05 | 9.88E−03 |
| Alad | 12 | 0.184 | 0.271 | −0.500 | 3.57E−05 | 1.08E−02 |
| Al413582 | 12 | 0.091 | 0.168 | −0.699 | 3.74E−05 | 1.12E−02 |
| Mat2b | 12 | 0.111 | 0.2 | −0.636 | 3.84E−05 | 1.15E−02 |
| Polr2i | 12 | 0.104 | 0.171 | −0.674 | 3.93E−05 | 1.18E−02 |
| Icos | 12 | 0.281 | 0.386 | −0.356 | 4.44E−05 | 1.31E−02 |
| Rbms1 | 12 | 0.226 | 0.268 | −0.300 | 4.91E−05 | 1.42E−02 |
| Cstb | 12 | 0.197 | 0.253 | −0.548 | 4.94E−05 | 1.43E−02 |
| Atp5g2 | 12 | 0.552 | 0.448 | 0.219 | 4.99E−05 | 1.44E−02 |
| Tmem176a | 12 | 0.199 | 0.293 | −0.544 | 5.21E−05 | 1.49E−02 |
| Cd27 | 12 | 0.402 | 0.293 | 0.409 | 5.26E−05 | 1.51E−02 |
| Aprt | 12 | 0.284 | 0.182 | 0.449 | 5.37E−05 | 1.54E−02 |
| Lbr | 12 | 0.148 | 0.211 | −0.498 | 5.49E−05 | 1.57E−02 |
| Rasa2 | 12 | 0.184 | 0.098 | 0.665 | 5.87E−05 | 1.66E−02 |
| Ypel3 | 12 | 0.375 | 0.483 | −0.375 | 6.04E−05 | 1.70E−02 |
| Ndufs7 | 12 | 0.21 | 0.281 | −0.413 | 6.04E−05 | 1.70E−02 |
| Tapbpl | 12 | 0.099 | 0.162 | −0.629 | 6.06E−05 | 1.70E−02 |
| G3bp1 | 12 | 0.239 | 0.317 | −0.433 | 6.09E−05 | 1.71E−02 |
| Rassf5 | 12 | 0.247 | 0.155 | 0.458 | 6.58E−05 | 1.83E−02 |
| Zc3h15 | 12 | 0.21 | 0.124 | 0.524 | 6.80E−05 | 1.89E−02 |
| Arpc4 | 12 | 0.39 | 0.301 | 0.362 | 6.96E−05 | 1.92E−02 |
| Ppp1r10 | 12 | 0.11 | 0.164 | −0.520 | 6.99E−05 | 1.93E−02 |
| Dusp11 | 12 | 0.217 | 0.126 | 0.563 | 7.03E−05 | 1.94E−02 |
| Cd37 | 12 | 0.332 | 0.231 | 0.368 | 7.13E−05 | 1.96E−02 |
| Tmem14c | 12 | 0.127 | 0.184 | −0.538 | 7.16E−05 | 1.96E−02 |
| Hnrnpu | 12 | 0.281 | 0.193 | 0.435 | 7.90E−05 | 2.14E−02 |
| Ptprc | 12 | 0.441 | 0.333 | 0.327 | 8.06E−05 | 2.18E−02 |
| Abracl | 12 | 0.357 | 0.251 | 0.406 | 8.08E−05 | 2.18E−02 |
| Cisd2 | 12 | 0.388 | 0.288 | 0.253 | 8.08E−05 | 2.18E−02 |
| Ikzf1 | 12 | 0.226 | 0.322 | −0.460 | 8.56E−05 | 2.28E−02 |
| Hcst | 12 | 0.437 | 0.33 | 0.253 | 8.85E−05 | 2.35E−02 |
| Elovl5 | 12 | 0.32 | 0.242 | 0.152 | 8.97E−05 | 2.38E−02 |
| Rnf166 | 12 | 0.131 | 0.199 | −0.569 | 8.98E−05 | 2.38E−02 |
| Bst2 | 12 | 0.086 | 0.16 | −0.675 | 9.00E−05 | 2.38E−02 |
| Polr2k | 12 | 0.301 | 0.213 | 0.266 | 9.18E−05 | 2.42E−02 |
| Med28 | 12 | 0.32 | 0.337 | −0.202 | 9.36E−05 | 2.46E−02 |
| Baz1a | 12 | 0.256 | 0.162 | 0.496 | 9.63E−05 | 2.52E−02 |
| Ifnar1 | 12 | 0.209 | 0.308 | −0.459 | 9.91E−05 | 2.58E−02 |
| Gpcpd1 | 12 | 0.107 | 0.182 | −0.607 | 1.02E−04 | 2.66E−02 |
| Zfand5 | 12 | 0.157 | 0.242 | −0.525 | 1.03E−04 | 2.66E−02 |
| Gbp7 | 12 | 0.115 | 0.199 | −0.681 | 1.03E−04 | 2.67E−02 |
| Rsrc2 | 12 | 0.24 | 0.299 | −0.365 | 1.05E−04 | 2.72E−02 |
| Birc3 | 12 | 0.255 | 0.27 | −0.292 | 1.07E−04 | 2.75E−02 |
| Elf1 | 12 | 0.378 | 0.397 | −0.249 | 1.16E−04 | 2.95E−02 |
| Ipmk | 12 | 0.226 | 0.138 | 0.375 | 1.30E−04 | 3.26E−02 |
| Tmed9 | 12 | 0.207 | 0.282 | −0.424 | 1.36E−04 | 3.38E−02 |
| Rab37 | 12 | 0.119 | 0.188 | −0.598 | 1.42E−04 | 3.51E−02 |
| Sumo3 | 12 | 0.119 | 0.191 | −0.580 | 1.58E−04 | 3.82E−02 |
| Pura | 12 | 0.103 | 0.179 | −0.588 | 1.59E−04 | 3.84E−02 |
| Ifrd1 | 12 | 0.428 | 0.536 | −0.210 | 1.60E−04 | 3.84E−02 |
| Btf3 | 12 | 0.716 | 0.643 | 0.151 | 1.61E−04 | 3.88E−02 |
| Zc3hav1 | 12 | 0.336 | 0.393 | −0.304 | 1.62E−04 | 3.90E−02 |
| Map1lc3b | 12 | 0.415 | 0.505 | −0.322 | 1.73E−04 | 4.14E−02 |
| Bnip3l | 12 | 0.281 | 0.346 | −0.334 | 1.76E−04 | 4.18E−02 |
| Timm13 | 12 | 0.244 | 0.324 | −0.449 | 1.76E−04 | 4.19E−02 |
| Cox17 | 12 | 0.436 | 0.352 | 0.301 | 1.88E−04 | 4.42E−02 |
| Gpr171 | 12 | 0.24 | 0.151 | 0.538 | 1.92E−04 | 4.50E−02 |
| Rela | 12 | 0.122 | 0.193 | −0.563 | 1.97E−04 | 4.60E−02 |
| Pim3 | 12 | 0.196 | 0.12 | 0.737 | 2.02E−04 | 4.70E−02 |
| Ccr4 | 12 | 0.166 | 0.087 | 0.675 | 2.09E−04 | 4.84E−02 |
| Mcpt1 | 13 | 0.31 | 0.014 | 4.351 | 6.62E−07 | 3.21E−04 |
| Mcpt2 | 13 | 0.319 | 0.028 | 3.840 | 8.50E−06 | 3.08E−03 |
| Vps37b | 13 | 0.168 | 0.514 | −1.238 | 3.79E−05 | 1.14E−02 |
| Malat1 | 13 | 0.956 | 1 | −0.616 | 4.18E−05 | 1.24E−02 |
| Prkca | 13 | 0.053 | 0.292 | −1.342 | 8.86E−05 | 2.35E−02 |
| Mcpt4 | 13 | 0.212 | 0.014 | 2.758 | 1.22E−04 | 3.09E−02 |
| Btg1 | 13 | 0.664 | 0.847 | −0.619 | 1.41E−04 | 3.49E−02 |
| Actb | 13 | 0.991 | 0.986 | 0.469 | 1.42E−04 | 3.51E−02 |
| Igkc | 13 | 0.973 | 0.931 | 1.825 | 2.03E−04 | 4.72E−02 |
| Hspa1a | 14 | 0.313 | 0.039 | 3.041 | 1.60E−28 | 7.81E−25 |
| Ighg1 | 14 | 0.232 | 0.013 | 3.120 | 3.10E−26 | 1.29E−22 |
| Tnfaip2 | 14 | 0.367 | 0.703 | −1.159 | 3.56E−24 | 1.24E−20 |
| Hspa1b | 14 | 0.181 | 0.009 | 3.382 | 1.25E−20 | 3.34E−17 |
| Mcpt1 | 14 | 0.201 | 0.009 | 4.922 | 2.24E−19 | 5.41E−16 |
| Iglc1 | 14 | 0.444 | 0.672 | −0.883 | 3.05E−19 | 7.27E−16 |
| Mcpt2 | 14 | 0.17 | 0.006 | 4.456 | 7.66E−18 | 1.58E−14 |
| Btg2 | 14 | 0.977 | 0.985 | −0.289 | 3.88E−17 | 7.46E−14 |
| Actg1 | 14 | 1 | 1 | 0.419 | 6.17E−17 | 1.16E−13 |
| Igkv1-135 | 14 | 0.058 | 0.239 | −1.531 | 6.21E−14 | 8.71E−11 |
| Zfp36 | 14 | 0.938 | 0.88 | 0.502 | 3.47E−12 | 3.96E−09 |
| Dab2 | 14 | 0.201 | 0.053 | 1.466 | 9.95E−12 | 1.08E−08 |
| Pilra | 14 | 0.359 | 0.565 | −0.586 | 5.41E−11 | 5.42E−08 |
| Hspa8 | 14 | 0.992 | 0.98 | 0.479 | 6.08E−11 | 6.05E−08 |
| Xlr | 14 | 0.278 | 0.483 | −0.746 | 9.04E−11 | 8.77E−08 |
| Ccr1 | 14 | 0.317 | 0.532 | −0.504 | 1.54E−10 | 1.45E−07 |
| Fosb | 14 | 0.564 | 0.746 | −0.494 | 1.88E−10 | 1.74E−07 |
| Nr4a2 | 14 | 0.483 | 0.68 | −0.509 | 3.73E−10 | 3.27E−07 |
| Ppia | 14 | 0.996 | 0.982 | 0.386 | 3.86E−10 | 3.38E−07 |
| Neat1 | 14 | 0.834 | 0.674 | 0.555 | 5.52E−10 | 4.76E−07 |
| Jun | 14 | 0.687 | 0.453 | 0.769 | 5.88E−10 | 5.02E−07 |
| Id2 | 14 | 0.514 | 0.324 | 0.997 | 6.84E−10 | 5.79E−07 |
| Hsp90aa1 | 14 | 0.714 | 0.562 | 0.738 | 9.01E−10 | 7.57E−07 |
| Arl4c | 14 | 0.344 | 0.51 | −0.709 | 1.01E−09 | 8.37E−07 |
| Srsf5 | 14 | 0.822 | 0.851 | −0.304 | 1.24E−09 | 1.01E−06 |
| Lgals3 | 14 | 0.892 | 0.913 | −0.323 | 1.36E−09 | 1.09E−06 |
| Agpat4 | 14 | 0.436 | 0.593 | −0.568 | 2.57E−09 | 1.96E−06 |
| Vps37b | 14 | 0.27 | 0.459 | −0.748 | 2.60E−09 | 1.99E−06 |
| Il1b | 14 | 0.749 | 0.891 | −0.287 | 3.12E−09 | 2.33E−06 |
| Cxcr4 | 14 | 0.456 | 0.223 | 1.002 | 4.01E−09 | 2.93E−06 |
| S100a6 | 14 | 0.707 | 0.448 | 0.622 | 8.77E−09 | 6.07E−06 |
| Arid5a | 14 | 0.251 | 0.411 | −0.640 | 9.84E−09 | 6.75E−06 |
| Hsp90ab1 | 14 | 0.961 | 0.893 | 0.435 | 1.37E−08 | 9.20E−06 |
| Fam110a | 14 | 0.212 | 0.343 | −0.815 | 2.00E−08 | 1.31E−05 |
| Zfp36l1 | 14 | 0.629 | 0.468 | 0.618 | 2.43E−08 | 1.56E−05 |
| Ebi3 | 14 | 0.166 | 0.32 | −0.957 | 2.66E−08 | 1.70E−05 |
| Fam43a | 14 | 0.066 | 0.201 | −1.331 | 2.73E−08 | 1.74E−05 |
| Il1r2 | 14 | 0.618 | 0.781 | −0.400 | 2.84E−08 | 1.81E−05 |
| Ifi30 | 14 | 0.911 | 0.812 | 0.500 | 3.14E−08 | 1.98E−05 |
| Gp2 | 14 | 0.089 | 0.225 | −1.351 | 3.20E−08 | 2.01E−05 |
| Lin54 | 14 | 0.131 | 0.256 | −0.872 | 3.81E−08 | 2.35E−05 |
| Igkv12-44 | 14 | 0.174 | 0.042 | 1.266 | 3.88E−08 | 2.39E−05 |
| Napsa | 14 | 0.942 | 0.858 | 0.442 | 4.76E−08 | 2.89E−05 |
| Cfp | 14 | 0.873 | 0.72 | 0.517 | 5.80E−08 | 3.45E−05 |
| Zfand5 | 14 | 0.66 | 0.766 | −0.528 | 6.61E−08 | 3.89E−05 |
| Ppp1r1a | 14 | 0.166 | 0.319 | −0.969 | 7.77E−08 | 4.51E−05 |
| Ifrd1 | 14 | 0.707 | 0.792 | −0.477 | 7.82E−08 | 4.54E−05 |
| Lacc1 | 14 | 0.104 | 0.215 | −0.999 | 8.10E−08 | 4.67E−05 |
| Syngr2 | 14 | 0.903 | 0.75 | 0.460 | 1.23E−07 | 6.93E−05 |
| Rgs2 | 14 | 0.622 | 0.751 | −0.310 | 1.59E−07 | 8.79E−05 |
| Gapdh | 14 | 0.946 | 0.904 | 0.411 | 2.77E−07 | 1.45E−04 |
| Fth1 | 14 | 1 | 1 | −0.206 | 4.16E−07 | 2.11E−04 |
| Icam1 | 14 | 0.44 | 0.486 | −0.618 | 5.47E−07 | 2.70E−04 |
| Ptp4a1 | 14 | 0.595 | 0.696 | −0.383 | 8.24E−07 | 3.90E−04 |
| Tyrobp | 14 | 0.961 | 0.971 | −0.247 | 9.57E−07 | 4.47E−04 |
| Klf4 | 14 | 0.32 | 0.462 | −0.399 | 1.19E−06 | 5.44E−04 |
| Hck | 14 | 0.336 | 0.44 | −0.460 | 1.48E−06 | 6.57E−04 |
| S100a4 | 14 | 0.324 | 0.151 | 0.620 | 1.50E−06 | 6.67E−04 |
| Ltc4s | 14 | 0.124 | 0.252 | −0.891 | 1.75E−06 | 7.63E−04 |
| Ms4a4c | 14 | 0.189 | 0.083 | 1.398 | 1.80E−06 | 7.85E−04 |
| Tiparp | 14 | 0.151 | 0.295 | −0.903 | 1.82E−06 | 7.90E−04 |
| Cxcl16 | 14 | 0.506 | 0.643 | −0.436 | 1.87E−06 | 8.11E−04 |
| Cd33 | 14 | 0.247 | 0.383 | −0.680 | 1.93E−06 | 8.30E−04 |
| Hepacam2 | 14 | 0.054 | 0.164 | −1.349 | 2.42E−06 | 1.02E−03 |
| Prdm1 | 14 | 0.042 | 0.16 | −1.404 | 2.46E−06 | 1.03E−03 |
| Atp5b | 14 | 0.919 | 0.805 | 0.377 | 2.48E−06 | 1.04E−03 |
| DynIl1 | 14 | 0.9 | 0.777 | 0.382 | 2.55E−06 | 1.06E−03 |
| Ckb | 14 | 0.784 | 0.606 | 0.493 | 2.87E−06 | 1.17E−03 |
| Clec4n | 14 | 0.247 | 0.413 | −0.710 | 3.02E−06 | 1.23E−03 |
| Slc25a5 | 14 | 0.938 | 0.88 | 0.383 | 3.32E−06 | 1.33E−03 |
| Atp5c1 | 14 | 0.88 | 0.775 | 0.382 | 3.73E−06 | 1.47E−03 |
| Plet1 | 14 | 0.293 | 0.438 | −0.497 | 3.83E−06 | 1.51E−03 |
| Hspd1 | 14 | 0.556 | 0.359 | 0.665 | 3.83E−06 | 1.51E−03 |
| Atp5h | 14 | 0.923 | 0.827 | 0.331 | 3.98E−06 | 1.56E−03 |
| Zfp263 | 14 | 0.398 | 0.514 | −0.502 | 4.63E−06 | 1.79E−03 |
| AW112010 | 14 | 0.382 | 0.516 | −0.201 | 4.80E−06 | 1.86E−03 |
| AF251705 | 14 | 0.363 | 0.49 | −0.523 | 5.32E−06 | 2.03E−03 |
| Nfkbiz | 14 | 0.436 | 0.556 | −0.644 | 6.23E−06 | 2.34E−03 |
| Ndel1 | 14 | 0.363 | 0.481 | −0.517 | 6.57E−06 | 2.45E−03 |
| Klf6 | 14 | 0.718 | 0.61 | 0.429 | 7.19E−06 | 2.66E−03 |
| Tubb5 | 14 | 0.768 | 0.6 | 0.542 | 7.30E−06 | 2.70E−03 |
| Spg21 | 14 | 0.486 | 0.567 | −0.361 | 7.69E−06 | 2.82E−03 |
| Tmem50b | 14 | 0.29 | 0.413 | −0.480 | 9.27E−06 | 3.33E−03 |
| Ak2 | 14 | 0.537 | 0.394 | 0.597 | 1.05E−05 | 3.72E−03 |
| Lair1 | 14 | 0.12 | 0.206 | −0.772 | 1.12E−05 | 3.95E−03 |
| Atf3 | 14 | 0.548 | 0.698 | −0.156 | 1.17E−05 | 4.10E−03 |
| Irf1 | 14 | 0.135 | 0.262 | −1.045 | 1.19E−05 | 4.13E−03 |
| Tnni2 | 14 | 0.722 | 0.803 | −0.164 | 1.33E−05 | 4.59E−03 |
| Sdc4 | 14 | 0.205 | 0.087 | 0.976 | 1.42E−05 | 4.85E−03 |
| Zbtb46 | 14 | 0.22 | 0.094 | 0.654 | 1.43E−05 | 4.87E−03 |
| Ninj1 | 14 | 0.201 | 0.348 | −0.685 | 1.47E−05 | 5.01E−03 |
| Tuba1b | 14 | 0.761 | 0.545 | 0.479 | 1.50E−05 | 5.10E−03 |
| Clta | 14 | 0.884 | 0.807 | 0.346 | 1.52E−05 | 5.16E−03 |
| Nlrp3 | 14 | 0.301 | 0.433 | −0.656 | 1.62E−05 | 5.46E−03 |
| Neurl3 | 14 | 0.479 | 0.597 | −0.385 | 1.73E−05 | 5.76E−03 |
| Mt1 | 14 | 0.448 | 0.617 | −0.720 | 1.73E−05 | 5.76E−03 |
| Tkt | 14 | 0.672 | 0.499 | 0.513 | 1.91E−05 | 6.27E−03 |
| Erlin1 | 14 | 0.158 | 0.274 | −0.606 | 1.96E−05 | 6.43E−03 |
| Dph3 | 14 | 0.336 | 0.416 | −0.452 | 2.08E−05 | 6.76E−03 |
| Rnaset2a | 14 | 0.757 | 0.775 | −0.247 | 2.21E−05 | 7.10E−03 |
| Rasgef1b | 14 | 0.541 | 0.348 | 0.369 | 2.26E−05 | 7.23E−03 |
| Rhob | 14 | 0.255 | 0.129 | 1.055 | 2.32E−05 | 7.43E−03 |
| Iglv1 | 14 | 0.093 | 0.186 | −0.608 | 2.33E−05 | 7.44E−03 |
| Fgl2 | 14 | 0.452 | 0.571 | −0.427 | 2.51E−05 | 7.94E−03 |
| Gsg1 | 14 | 0.093 | 0.204 | −0.956 | 2.58E−05 | 8.14E−03 |
| Rap1b | 14 | 0.842 | 0.816 | −0.198 | 2.62E−05 | 8.21E−03 |
| Npm1 | 14 | 0.958 | 0.906 | 0.310 | 2.79E−05 | 8.67E−03 |
| Tm2d2 | 14 | 0.502 | 0.558 | −0.386 | 2.87E−05 | 8.93E−03 |
| Lmo2 | 14 | 0.49 | 0.578 | −0.320 | 2.90E−05 | 9.00E−03 |
| Pcyt2 | 14 | 0.189 | 0.293 | −0.536 | 2.94E−05 | 9.12E−03 |
| Runx1 | 14 | 0.274 | 0.379 | −0.623 | 3.24E−05 | 9.89E−03 |
| Ppp1r15a | 14 | 0.653 | 0.595 | −0.405 | 3.28E−05 | 1.00E−02 |
| Sla | 14 | 0.633 | 0.49 | 0.504 | 3.29E−05 | 1.00E−02 |
| Crem | 14 | 0.355 | 0.455 | −0.536 | 3.78E−05 | 1.14E−02 |
| Pa2g4 | 14 | 0.583 | 0.372 | 0.564 | 3.90E−05 | 1.17E−02 |
| Vim | 14 | 0.923 | 0.84 | 0.314 | 4.07E−05 | 1.21E−02 |
| Dusp1 | 14 | 0.838 | 0.759 | 0.380 | 4.34E−05 | 1.28E−02 |
| Ltb | 14 | 0.618 | 0.573 | 0.582 | 4.68E−05 | 1.36E−02 |
| Ctage5 | 14 | 0.498 | 0.6 | −0.293 | 4.91E−05 | 1.42E−02 |
| Nuak2 | 14 | 0.208 | 0.331 | −0.566 | 5.87E−05 | 1.66E−02 |
| Btg1 | 14 | 0.973 | 0.989 | −0.221 | 5.87E−05 | 1.66E−02 |
| Cxx1b | 14 | 0.228 | 0.368 | −0.572 | 6.28E−05 | 1.75E−02 |
| Aif1 | 14 | 0.363 | 0.495 | −0.259 | 6.54E−05 | 1.82E−02 |
| Herpud1 | 14 | 0.703 | 0.737 | −0.254 | 6.61E−05 | 1.84E−02 |
| Ifnar1 | 14 | 0.309 | 0.401 | −0.505 | 7.81E−05 | 2.12E−02 |
| Atp2c1 | 14 | 0.236 | 0.103 | 0.828 | 7.87E−05 | 2.13E−02 |
| Mmp12 | 14 | 0.097 | 0.214 | −0.837 | 7.92E−05 | 2.14E−02 |
| Ntpcr | 14 | 0.166 | 0.274 | −0.631 | 8.29E−05 | 2.22E−02 |
| Cd209a | 14 | 0.436 | 0.622 | −0.315 | 8.53E−05 | 2.28E−02 |
| Slc25a33 | 14 | 0.154 | 0.247 | −0.661 | 1.07E−04 | 2.75E−02 |
| Smim3 | 14 | 0.568 | 0.606 | −0.317 | 1.09E−04 | 2.81E−02 |
| Nfkbia | 14 | 0.896 | 0.775 | 0.243 | 1.12E−04 | 2.87E−02 |
| Baiap2 | 14 | 0.197 | 0.308 | −0.610 | 1.16E−04 | 2.97E−02 |
| Atf4 | 14 | 0.672 | 0.654 | −0.231 | 1.18E−04 | 3.01E−02 |
| Cd300a | 14 | 0.571 | 0.635 | −0.305 | 1.24E−04 | 3.14E−02 |
| Csnk2b | 14 | 0.591 | 0.435 | 0.458 | 1.26E−04 | 3.19E−02 |
| Mgl2 | 14 | 0.212 | 0.173 | 0.811 | 1.34E−04 | 3.36E−02 |
| Dnase1l3 | 14 | 0.232 | 0.365 | −0.624 | 1.37E−04 | 3.41E−02 |
| Arih1 | 14 | 0.32 | 0.396 | −0.585 | 1.40E−04 | 3.46E−02 |
| Serpinb1a | 14 | 0.224 | 0.088 | 0.860 | 1.44E−04 | 3.55E−02 |
| Cd7 | 14 | 0.351 | 0.249 | 0.893 | 1.45E−04 | 3.56E−02 |
| Csf2rb | 14 | 0.436 | 0.573 | −0.453 | 1.51E−04 | 3.68E−02 |
| Fndc5 | 14 | 0.151 | 0.252 | −0.668 | 1.52E−04 | 3.70E−02 |
| Tspan13 | 14 | 0.745 | 0.814 | −0.189 | 1.56E−04 | 3.78E−02 |
| Tifab | 14 | 0.228 | 0.319 | −0.483 | 1.59E−04 | 3.84E−02 |
| Rnf149 | 14 | 0.625 | 0.7 | −0.352 | 1.75E−04 | 4.16E−02 |
| Ccl3 | 14 | 0.174 | 0.077 | 1.191 | 1.75E−04 | 4.18E−02 |
| St13 | 14 | 0.614 | 0.418 | 0.430 | 1.85E−04 | 4.35E−02 |
| Pglyrp1 | 14 | 0.398 | 0.245 | 0.794 | 1.97E−04 | 4.60E−02 |
| Gsdmd | 14 | 0.228 | 0.147 | 0.638 | 2.13E−04 | 4.94E−02 |
| Sla | 15 | 0.617 | 0.335 | 0.970 | 3.56E−64 | 1.15E−59 |
| Jund | 15 | 0.797 | 0.928 | −0.492 | 2.47E−59 | 5.13E−55 |
| Klf4 | 15 | 0.104 | 0.319 | −1.292 | 2.04E−46 | 2.47E−42 |
| Dusp1 | 15 | 0.492 | 0.729 | −0.680 | 1.33E−45 | 1.49E−41 |
| Btg2 | 15 | 0.779 | 0.897 | −0.347 | 5.17E−33 | 3.27E−29 |
| Fosb | 15 | 0.212 | 0.416 | −0.826 | 5.80E−33 | 3.63E−29 |
| Pim1 | 15 | 0.406 | 0.576 | −0.561 | 3.11E−26 | 1.29E−22 |
| Kdm6b | 15 | 0.315 | 0.496 | −0.670 | 7.03E−25 | 2.62E−21 |
| Irf7 | 15 | 0.06 | 0.176 | −1.344 | 1.60E−23 | 5.23E−20 |
| Junb | 15 | 0.938 | 0.982 | −0.162 | 3.33E−23 | 1.05E−19 |
| Bcl2 | 15 | 0.434 | 0.288 | 0.691 | 1.02E−22 | 3.14E−19 |
| Igkc | 15 | 0.967 | 0.933 | 0.581 | 1.53E−22 | 4.65E−19 |
| Hk2 | 15 | 0.158 | 0.283 | −0.866 | 1.65E−21 | 4.65E−18 |
| Ptpn22 | 15 | 0.473 | 0.305 | 0.582 | 1.12E−20 | 3.02E−17 |
| Pnrc1 | 15 | 0.506 | 0.648 | −0.383 | 1.20E−20 | 3.21E−17 |
| Fos | 15 | 0.627 | 0.765 | −0.368 | 3.15E−20 | 8.25E−17 |
| Ppp1r15a | 15 | 0.183 | 0.323 | −0.710 | 4.02E−19 | 9.44E−16 |
| Phlda1 | 15 | 0.157 | 0.287 | −0.769 | 1.45E−18 | 3.24E−15 |
| Srgn | 15 | 0.568 | 0.422 | 0.478 | 7.07E−18 | 1.46E−14 |
| Zfp36l2 | 15 | 0.364 | 0.219 | 0.671 | 8.48E−18 | 1.74E−14 |
| Prr7 | 15 | 0.08 | 0.19 | −0.972 | 1.57E−17 | 3.13E−14 |
| Rsrp1 | 15 | 0.299 | 0.421 | −0.508 | 2.68E−17 | 5.24E−14 |
| Igha | 15 | 0.905 | 0.83 | 0.540 | 5.48E−16 | 9.49E−13 |
| Iglc1 | 15 | 0.104 | 0.217 | −0.765 | 6.61E−16 | 1.14E−12 |
| Hsp90aa1 | 15 | 0.283 | 0.163 | 0.761 | 7.31E−16 | 1.25E−12 |
| Vps37b | 15 | 0.387 | 0.497 | −0.461 | 8.21E−16 | 1.39E−12 |
| Emb | 15 | 0.563 | 0.676 | −0.344 | 1.93E−15 | 3.16E−12 |
| Cxcr6 | 15 | 0.651 | 0.512 | 0.363 | 2.72E−15 | 4.45E−12 |
| Klrk1 | 15 | 0.319 | 0.446 | −0.502 | 2.95E−15 | 4.79E−12 |
| Slc7a6os | 15 | 0.074 | 0.16 | −0.957 | 6.70E−15 | 1.04E−11 |
| Ifrd1 | 15 | 0.195 | 0.304 | −0.605 | 1.36E−14 | 2.05E−11 |
| Tgif1 | 15 | 0.263 | 0.391 | −0.476 | 4.15E−14 | 5.91E−11 |
| Tnfaip3 | 15 | 0.31 | 0.44 | −0.457 | 7.00E−14 | 9.75E−11 |
| Rgcc | 15 | 0.238 | 0.14 | 0.905 | 9.36E−14 | 1.28E−10 |
| Pabpc1 | 15 | 0.486 | 0.574 | −0.348 | 2.94E−13 | 3.80E−10 |
| BC031181 | 15 | 0.293 | 0.376 | −0.437 | 3.22E−13 | 4.13E−10 |
| Il22 | 15 | 0.119 | 0.211 | −1.132 | 4.47E−13 | 5.66E−10 |
| Ikzf2 | 15 | 0.26 | 0.335 | −0.468 | 2.23E−12 | 2.64E−09 |
| Rasl11a | 15 | 0.225 | 0.13 | 0.776 | 3.43E−12 | 3.93E−09 |
| 4930523C07Rik | 15 | 0.196 | 0.284 | −0.548 | 5.47E−12 | 6.12E−09 |
| Cd69 | 15 | 0.371 | 0.49 | −0.392 | 5.61E−12 | 6.27E−09 |
| Dok1 | 15 | 0.18 | 0.086 | 0.749 | 1.82E−11 | 1.93E−08 |
| Actg1 | 15 | 0.675 | 0.591 | 0.327 | 1.45E−10 | 1.37E−07 |
| Sqstm1 | 15 | 0.205 | 0.274 | −0.446 | 1.83E−10 | 1.70E−07 |
| Gimap1 | 15 | 0.403 | 0.287 | 0.434 | 2.95E−10 | 2.66E−07 |
| Ptp4a2 | 15 | 0.31 | 0.409 | −0.387 | 3.33E−10 | 2.97E−07 |
| Maff | 15 | 0.382 | 0.456 | −0.358 | 3.93E−10 | 3.42E−07 |
| Ubb | 15 | 0.85 | 0.837 | −0.164 | 1.26E−09 | 1.02E−06 |
| Btg1 | 15 | 0.778 | 0.832 | −0.196 | 1.30E−09 | 1.04E−06 |
| Icos | 15 | 0.169 | 0.25 | −0.490 | 4.08 E−09 | 2.98E−06 |
| Fcer1g | 15 | 0.714 | 0.606 | 0.250 | 5.49E−09 | 3.93E−06 |
| Slc3a2 | 15 | 0.308 | 0.21 | 0.482 | 6.20E−09 | 4.42E−06 |
| Il2rg | 15 | 0.43 | 0.508 | −0.294 | 7.60E−09 | 5.32E−06 |
| Uhrf2 | 15 | 0.335 | 0.429 | −0.336 | 8.61E−09 | 5.97E−06 |
| Sepp1 | 15 | 0.639 | 0.689 | −0.213 | 1.11E−08 | 7.59E−06 |
| Nfkb1 | 15 | 0.162 | 0.247 | −0.489 | 1.53E−08 | 1.02E−05 |
| Sdhaf1 | 15 | 0.239 | 0.147 | 0.471 | 1.57E−08 | 1.05E−05 |
| Txk | 15 | 0.336 | 0.425 | −0.354 | 1.83E−08 | 1.21E−05 |
| Gimap5 | 15 | 0.277 | 0.184 | 0.508 | 1.85E−08 | 1.22E−05 |
| Mmd | 15 | 0.206 | 0.122 | 0.613 | 2.05E−08 | 1.34E−05 |
| Gatad1 | 15 | 0.098 | 0.151 | −0.587 | 3.89E−08 | 2.39E−05 |
| Gpr157 | 15 | 0.161 | 0.086 | 0.658 | 3.97E−08 | 2.44E−05 |
| Ncoa7 | 15 | 0.388 | 0.281 | 0.372 | 4.04E−08 | 2.47E−05 |
| Nfkbia | 15 | 0.488 | 0.583 | −0.217 | 4.79E−08 | 2.89E−05 |
| Bhlhe40 | 15 | 0.428 | 0.512 | −0.275 | 5.09E−08 | 3.06E−05 |
| Zc3h12a | 15 | 0.146 | 0.215 | −0.484 | 8.73E−08 | 5.01E−05 |
| Tmem176a | 15 | 0.554 | 0.465 | 0.304 | 1.11E−07 | 6.27E−05 |
| Ptp4a1 | 15 | 0.152 | 0.202 | −0.423 | 1.23E−07 | 6.90E−05 |
| Ffar2 | 15 | 0.307 | 0.219 | 0.436 | 1.42E−07 | 7.92E−05 |
| Fam110a | 15 | 0.128 | 0.195 | −0.569 | 2.16E−07 | 1.16E−04 |
| Saraf | 15 | 0.267 | 0.331 | −0.335 | 2.39E−07 | 1.27E−04 |
| Gpr171 | 15 | 0.193 | 0.119 | 0.561 | 3.35E−07 | 1.73E−04 |
| Gem | 15 | 0.442 | 0.498 | −0.291 | 6.28E−07 | 3.06E−04 |
| Zfp36l1 | 15 | 0.797 | 0.734 | 0.223 | 7.29E−07 | 3.48E−04 |
| Tgoln1 | 15 | 0.171 | 0.22 | −0.390 | 7.70E−07 | 3.66E−04 |
| Abhd2 | 15 | 0.26 | 0.351 | −0.399 | 8.29E−07 | 3.92E−04 |
| Clcn3 | 15 | 0.159 | 0.222 | −0.424 | 1.02E−06 | 4.72E−04 |
| Lgals3 | 15 | 0.529 | 0.572 | −0.219 | 1.19E−06 | 5.41E−04 |
| H2-K1 | 15 | 0.593 | 0.622 | −0.181 | 1.25E−06 | 5.67E−04 |
| Nr4a3 | 15 | 0.176 | 0.106 | 0.618 | 1.38E−06 | 6.15E−04 |
| Ahr | 15 | 0.183 | 0.111 | 0.510 | 1.82E−06 | 7.92E−04 |
| Tmem176b | 15 | 0.612 | 0.517 | 0.252 | 2.19E−06 | 9.28E−04 |
| Nfkbiz | 15 | 0.183 | 0.244 | −0.394 | 2.22E−06 | 9.41E−04 |
| Litaf | 15 | 0.247 | 0.165 | 0.430 | 2.65E−06 | 1.09E−03 |
| Gimap9 | 15 | 0.158 | 0.095 | 0.576 | 2.96E−06 | 1.21E−03 |
| Clk1 | 15 | 0.296 | 0.364 | −0.309 | 3.26E−06 | 1.31E−03 |
| B930036N10Rik | 15 | 0.107 | 0.153 | −0.603 | 3.47E−06 | 1.39E−03 |
| Mast4 | 15 | 0.166 | 0.219 | −0.371 | 3.57E−06 | 1.42E−03 |
| Gltscr2 | 15 | 0.354 | 0.408 | −0.254 | 4.91E−06 | 1.89E−03 |
| Slc25a3 | 15 | 0.224 | 0.286 | −0.333 | 5.24E−06 | 2.01E−03 |
| Asb2 | 15 | 0.183 | 0.238 | −0.386 | 6.86E−06 | 2.55E−03 |
| Orai1 | 15 | 0.155 | 0.093 | 0.486 | 8.96E−06 | 3.23E−03 |
| Ubald2 | 15 | 0.13 | 0.173 | −0.455 | 9.14E−06 | 3.29E−03 |
| Pcbp2 | 15 | 0.361 | 0.419 | −0.234 | 9.64E−06 | 3.45E−03 |
| Fam107b | 15 | 0.186 | 0.118 | 0.509 | 1.09E−05 | 3.83E−03 |
| Xlr4c | 15 | 0.146 | 0.195 | −0.381 | 1.18E−05 | 4.12E−03 |
| Srsf5 | 15 | 0.48 | 0.538 | −0.179 | 1.23E−05 | 4.28E−03 |
| AW112010 | 15 | 0.776 | 0.706 | 0.183 | 1.26E−05 | 4.35E−03 |
| Dusp5 | 15 | 0.329 | 0.408 | −0.257 | 1.41E−05 | 4.81E−03 |
| Hspe1 | 15 | 0.253 | 0.178 | 0.384 | 1.69E−05 | 5.65E−03 |
| Mapkapk2 | 15 | 0.162 | 0.1 | 0.528 | 1.74E−05 | 5.78E−03 |
| Cd7 | 15 | 0.397 | 0.314 | 0.305 | 1.79E−05 | 5.93E−03 |
| Ccnl1 | 15 | 0.2 | 0.252 | −0.331 | 1.98E−05 | 6.48E−03 |
| Nfkbid | 15 | 0.15 | 0.212 | −0.427 | 2.15E−05 | 6.95E−03 |
| Prdx2 | 15 | 0.165 | 0.104 | 0.451 | 2.23E−05 | 7.14E−03 |
| Tnfsf11 | 15 | 0.204 | 0.135 | 0.422 | 2.38E−05 | 7.60E−03 |
| Ybx1 | 15 | 0.43 | 0.493 | −0.206 | 2.58E−05 | 8.13E−03 |
| Actb | 15 | 0.881 | 0.917 | −0.206 | 3.83E−05 | 1.15E−02 |
| Samsn1 | 15 | 0.334 | 0.252 | 0.264 | 4.01E−05 | 1.19E−02 |
| Hspa8 | 15 | 0.619 | 0.566 | 0.228 | 4.14E−05 | 1.23E−02 |
| Lcp1 | 15 | 0.399 | 0.321 | 0.335 | 4.25E−05 | 1.26E−02 |
| Dhrs3 | 15 | 0.224 | 0.155 | 0.385 | 4.92E−05 | 1.42E−02 |
| Kit | 15 | 0.295 | 0.337 | −0.236 | 5.11E−05 | 1.47E−02 |
| Cox17 | 15 | 0.349 | 0.282 | 0.293 | 5.34E−05 | 1.53E−02 |
| Ppm1h | 15 | 0.101 | 0.151 | −0.498 | 6.23E−05 | 1.75E−02 |
| Gna13 | 15 | 0.203 | 0.254 | −0.319 | 6.38E−05 | 1.78E−02 |
| Egr1 | 15 | 0.17 | 0.112 | 0.477 | 6.39E−05 | 1.78E−02 |
| Ccng2 | 15 | 0.143 | 0.194 | −0.396 | 7.04E−05 | 1.94E−02 |
| Tmem59 | 15 | 0.368 | 0.286 | 0.266 | 7.78E−05 | 2.11E−02 |
| Nabp1 | 15 | 0.355 | 0.422 | −0.242 | 8.55E−05 | 2.28E−02 |
| Pfdn5 | 15 | 0.603 | 0.523 | 0.204 | 9.03E−05 | 2.39E−02 |
| Selplg | 15 | 0.504 | 0.433 | 0.275 | 9.18E−05 | 2.42E−02 |
| H2-Q7 | 15 | 0.13 | 0.166 | −0.354 | 9.97E−05 | 2.59E−02 |
| Spop | 15 | 0.14 | 0.191 | −0.355 | 1.04E−04 | 2.70E−02 |
| Serp1 | 15 | 0.224 | 0.275 | −0.272 | 1.06E−04 | 2.75E−02 |
| Ptprcap | 15 | 0.327 | 0.252 | 0.323 | 1.08E−04 | 2.77E−02 |
| Ier5 | 15 | 0.25 | 0.313 | −0.262 | 1.23E−04 | 3.12E−02 |
| Calm2 | 15 | 0.41 | 0.444 | −0.186 | 1.25E−04 | 3.16E−02 |
| Coq10b | 15 | 0.192 | 0.255 | −0.308 | 1.35E−04 | 3.37E−02 |
| Kcnk1 | 15 | 0.22 | 0.158 | 0.396 | 1.37E−04 | 3.40E−02 |
| Zap70 | 15 | 0.243 | 0.182 | 0.350 | 1.38E−04 | 3.43E−02 |
| C4b | 15 | 0.227 | 0.161 | 0.410 | 1.51E−04 | 3.68E−02 |
| Rbm3 | 15 | 0.414 | 0.458 | −0.186 | 1.53E−04 | 3.72E−02 |
| Eif4a1 | 15 | 0.404 | 0.437 | −0.177 | 1.56E−04 | 3.77E−02 |
| Tmed2 | 15 | 0.296 | 0.348 | −0.228 | 1.56E−04 | 3.78E−02 |
| Hsp90ab1 | 15 | 0.363 | 0.31 | 0.316 | 1.84E−04 | 4.35E−02 |
| Dph3 | 15 | 0.137 | 0.175 | −0.315 | 2.00E−04 | 4.67E−02 |
| Gm2a | 15 | 0.226 | 0.279 | −0.336 | 2.01E−04 | 4.69E−02 |
| Rsrp1 | 16 | 0.328 | 0.676 | −1.081 | 6.67E−08 | 3.91E−05 |
| Btg2 | 16 | 0.681 | 0.901 | −0.858 | 8.99E−08 | 5.14E−05 |
| Traf1 | 16 | 0.138 | 0.341 | −0.795 | 1.19E−04 | 3.04E−02 |
| H2-D1 | 16 | 0.94 | 0.951 | −0.358 | 1.71E−04 | 4.10E−02 |
| Pnrc1 | 16 | 0.25 | 0.577 | −0.914 | 1.92E−04 | 4.50E−02 |
| Ifngr1 | 17 | 0.267 | 0.857 | −1.121 | 1.01E−04 | 2.62E−02 |
| Cd28 | 19 | 0.588 | 0.458 | −0.588 | 2.92E−06 | 1.19E−03 |
| Cd74 | 19 | 0.118 | 0.5 | −0.250 | 9.75E−06 | 3.48E−03 |
| Trbc1 | 19 | 0.706 | 0.333 | 2.193 | 1.69E−05 | 5.65E−03 |
| Igkv1-135 | 2 | 0.004 | 0.175 | −3.217 | 6.34E−08 | 3.74E−05 |
| Fosl2 | 2 | 0.032 | 0.175 | −1.947 | 2.21E−05 | 7.10E−03 |
| Nr4a2 | 2 | 0.035 | 0.175 | −2.145 | 4.72E−05 | 1.38E−02 |
| Gapdh | 2 | 0.603 | 0.54 | 0.677 | 1.04E−04 | 2.69E−02 |
| Txnip | 20 | 0.205 | 0.582 | −1.669 | 1.31E−184 | 7.60E−179 |
| Mcpt1 | 20 | 0.252 | 0.02 | 2.731 | 7.68E−163 | 2.23E−157 |
| Igkv1-135 | 20 | 0.091 | 0.365 | −1.523 | 1.15E−110 | 1.67E−105 |
| Mcpt2 | 20 | 0.159 | 0.007 | 3.011 | 5.12E−109 | 5.96E−104 |
| Ighg1 | 20 | 0.381 | 0.13 | 2.328 | 2.92E−108 | 2.83E−103 |
| Igkv12-44 | 20 | 0.243 | 0.072 | 1.451 | 3.22E−88 | 2.68E−83 |
| Ifi27l2a | 20 | 0.354 | 0.622 | −1.074 | 1.90E−84 | 1.10E−79 |
| Ubald2 | 20 | 0.484 | 0.734 | −0.802 | 1.92E−82 | 1.01E−77 |
| Cacna1s | 20 | 0.419 | 0.695 | −0.890 | 5.33E−79 | 2.59E−74 |
| Sepp1 | 20 | 0.235 | 0.501 | −0.909 | 6.73E−77 | 3.01E−72 |
| Pdia6 | 20 | 0.767 | 0.833 | 0.189 | 5.39E−64 | 1.65E−59 |
| Rilpl2 | 20 | 0.48 | 0.686 | −0.725 | 2.17E−63 | 6.31E−59 |
| Iglc2 | 20 | 0.892 | 0.96 | −0.559 | 1.71E−62 | 4.32E−58 |
| Dusp5 | 20 | 0.402 | 0.65 | −0.790 | 8.28E−62 | 1.93E−57 |
| Fosb | 20 | 0.527 | 0.761 | −0.671 | 5.45E−60 | 1.22E−55 |
| Serp1 | 20 | 0.88 | 0.975 | −0.376 | 5.65E−60 | 1.22E−55 |
| Grasp | 20 | 0.091 | 0.269 | −1.223 | 1.80E−55 | 3.37E−51 |
| Ccr10 | 20 | 0.711 | 0.681 | 0.296 | 5.19E−55 | 9.15E−51 |
| Ddit4 | 20 | 0.174 | 0.058 | 1.315 | 2.50E−53 | 4.15E−49 |
| Malat1 | 20 | 0.976 | 0.99 | −0.406 | 9.30E−53 | 1.46E−48 |
| Pim1 | 20 | 0.767 | 0.916 | −0.469 | 5.00E−49 | 6.93E−45 |
| Hsp90b1 | 20 | 0.896 | 0.96 | 0.166 | 1.25E−46 | 1.55E−42 |
| Trf | 20 | 0.566 | 0.779 | −0.516 | 3.72E−46 | 4.32E−42 |
| Ifi27 | 20 | 0.102 | 0.273 | −1.059 | 2.93E−45 | 3.22E−41 |
| Odc1 | 20 | 0.297 | 0.485 | −0.890 | 3.02E−44 | 3.14E−40 |
| Xbp1 | 20 | 0.862 | 0.962 | −0.460 | 3.30E−44 | 3.37E−40 |
| Rgcc | 20 | 0.18 | 0.372 | −0.945 | 5.83E−44 | 5.85E−40 |
| Nrip1 | 20 | 0.281 | 0.507 | −0.727 | 3.65E−42 | 3.48E−38 |
| Cd274 | 20 | 0.175 | 0.355 | −0.938 | 8.53E−42 | 8.00E−38 |
| Eif1 | 20 | 0.902 | 0.979 | −0.352 | 3.22E−41 | 2.93E−37 |
| Chac1 | 20 | 0.042 | 0.165 | −1.345 | 4.88E−40 | 4.24E−36 |
| Gmfg | 20 | 0.204 | 0.113 | 0.842 | 2.80E−39 | 2.36E−35 |
| Ly6a | 20 | 0.052 | 0.173 | −1.632 | 1.26E−38 | 1.02E−34 |
| Pycard | 20 | 0.669 | 0.725 | 0.345 | 6.97E−38 | 5.48E−34 |
| Iglc1 | 20 | 0.831 | 0.956 | −0.986 | 8.37E−38 | 6.50E−34 |
| Actg1 | 20 | 0.624 | 0.633 | 0.359 | 2.29E−37 | 1.73E−33 |
| Lmna | 20 | 0.094 | 0.233 | −1.156 | 5.66E−37 | 4.22E−33 |
| Mdh1 | 20 | 0.535 | 0.505 | 0.218 | 3.80E−35 | 2.63E−31 |
| Trib1 | 20 | 0.3 | 0.494 | −0.641 | 4.86E−35 | 3.33E−31 |
| Coro1a | 20 | 0.534 | 0.513 | 0.294 | 7.57E−35 | 5.12E−31 |
| Fth1 | 20 | 0.835 | 0.96 | −0.389 | 1.87E−34 | 1.25E−30 |
| Ifitm3 | 20 | 0.221 | 0.396 | −0.837 | 1.27E−32 | 7.64E−29 |
| Ly6d | 20 | 0.307 | 0.499 | −0.610 | 1.21E−31 | 6.96E−28 |
| Cdc42 | 20 | 0.601 | 0.587 | 0.173 | 1.82E−31 | 1.03E−27 |
| Ahnak | 20 | 0.13 | 0.274 | −0.857 | 2.40E−31 | 1.34E−27 |
| Clic4 | 20 | 0.322 | 0.513 | −0.601 | 6.74E−31 | 3.74E−27 |
| Iglc3 | 20 | 0.8 | 0.938 | −1.260 | 7.73E−31 | 4.20E−27 |
| Zfp36l2 | 20 | 0.37 | 0.292 | 0.651 | 1.30E−30 | 6.93E−27 |
| Mtdh | 20 | 0.65 | 0.847 | −0.428 | 1.89E−30 | 1.00E−26 |
| Atf4 | 20 | 0.537 | 0.711 | −0.482 | 6.56E−30 | 3.32E−26 |
| Rpn1 | 20 | 0.548 | 0.565 | 0.164 | 3.86E−29 | 1.92E−25 |
| Hsp90aa1 | 20 | 0.371 | 0.343 | 0.506 | 4.23E−28 | 2.02E−24 |
| Fkbp11 | 20 | 0.542 | 0.549 | 0.240 | 4.27E−28 | 2.02E−24 |
| Pkm | 20 | 0.53 | 0.547 | 0.177 | 4.79E−28 | 2.23E−24 |
| Txndc5 | 20 | 0.912 | 0.977 | −0.302 | 1.02E−27 | 4.58E−24 |
| 5031425E22Rik | 20 | 0.319 | 0.487 | −0.660 | 1.51E−27 | 6.75E−24 |
| Ralgds | 20 | 0.079 | 0.195 | −0.829 | 1.84E−26 | 7.80E−23 |
| Vps37b | 20 | 0.078 | 0.196 | −0.936 | 2.42E−26 | 1.02E−22 |
| Zfp706 | 20 | 0.689 | 0.852 | −0.388 | 3.43E−26 | 1.41E−22 |
| Eif4ebp1 | 20 | 0.149 | 0.279 | −0.838 | 4.71E−26 | 1.92E−22 |
| Il10rb | 20 | 0.181 | 0.316 | −0.652 | 5.08E−26 | 2.05E−22 |
| Mcl1 | 20 | 0.505 | 0.683 | −0.512 | 7.71E−26 | 3.05E−22 |
| Ifnar2 | 20 | 0.216 | 0.359 | −0.632 | 1.85E−25 | 7.18E−22 |
| Tmem154 | 20 | 0.256 | 0.429 | −0.595 | 2.32E−25 | 8.89E−22 |
| Eno1 | 20 | 0.278 | 0.22 | 0.377 | 6.47E−25 | 2.43E−21 |
| Ccser2 | 20 | 0.199 | 0.344 | −0.660 | 1.25E−24 | 4.54E−21 |
| Gm | 20 | 0.2 | 0.373 | −0.547 | 1.41E−24 | 5.09E−21 |
| Taldo1 | 20 | 0.497 | 0.488 | 0.159 | 1.59E−24 | 5.73E−21 |
| Ost4 | 20 | 0.756 | 0.895 | −0.347 | 2.35E−24 | 8.39E−21 |
| Ifi30 | 20 | 0.201 | 0.133 | 0.502 | 2.46E−24 | 8.72E−21 |
| Bst2 | 20 | 0.546 | 0.71 | −0.454 | 2.90E−24 | 1.02E−20 |
| Igkv3-7 | 20 | 0.183 | 0.1 | 0.872 | 3.30E−24 | 1.16E−20 |
| Slc7a5 | 20 | 0.125 | 0.259 | −0.798 | 8.26E−24 | 2.83E−20 |
| Kcnmb4os2 | 20 | 0.224 | 0.382 | −0.548 | 1.35E−23 | 4.47E−20 |
| Ptprcap | 20 | 0.545 | 0.528 | 0.192 | 1.47E−23 | 4.82E−20 |
| Ifnar1 | 20 | 0.139 | 0.27 | −0.725 | 1.83E−23 | 5.92E−20 |
| Plac8 | 20 | 0.119 | 0.252 | −0.724 | 2.61E−23 | 8.29E−20 |
| Ly6c1 | 20 | 0.858 | 0.94 | −0.411 | 3.92E−23 | 1.22E−19 |
| Tnfrsf13b | 20 | 0.562 | 0.75 | −0.406 | 6.67E−23 | 2.07E−19 |
| Uqcrc1 | 20 | 0.477 | 0.459 | 0.175 | 2.42E−22 | 7.19E−19 |
| Gimap5 | 20 | 0.306 | 0.461 | −0.561 | 3.33E−22 | 9.85E−19 |
| Ssbp1 | 20 | 0.254 | 0.378 | −0.558 | 3.79E−22 | 1.11E−18 |
| Prg2 | 20 | 0.147 | 0.293 | −0.555 | 3.92E−22 | 1.15E−18 |
| Peli1 | 20 | 0.273 | 0.423 | −0.593 | 4.10E−22 | 1.19E−18 |
| Ftl1 | 20 | 0.942 | 0.991 | −0.270 | 4.52E−22 | 1.31E−18 |
| Limd2 | 20 | 0.509 | 0.685 | −0.439 | 1.09E−21 | 3.12E−18 |
| Bckdk | 20 | 0.399 | 0.589 | −0.476 | 1.60E−21 | 4.51E−18 |
| Nfkbia | 20 | 0.691 | 0.829 | −0.460 | 6.97E−21 | 1.90E−17 |
| Saraf | 20 | 0.421 | 0.579 | −0.444 | 1.89E−20 | 4.99E−17 |
| Ly6c2 | 20 | 0.909 | 0.967 | −0.370 | 2.15E−20 | 5.67E−17 |
| Fxyd5 | 20 | 0.499 | 0.468 | 0.193 | 3.27E−20 | 8.49E−17 |
| P2rx4 | 20 | 0.136 | 0.263 | −0.715 | 4.18E−20 | 1.07E−16 |
| Cd69 | 20 | 0.557 | 0.755 | −0.375 | 5.37E−20 | 1.37E−16 |
| Ppp1r15a | 20 | 0.372 | 0.508 | −0.541 | 5.72E−20 | 1.45E−16 |
| Ifrd1 | 20 | 0.202 | 0.351 | −0.601 | 7.49E−20 | 1.89E−16 |
| Itgb7 | 20 | 0.507 | 0.503 | 0.210 | 8.04E−20 | 2.02E−16 |
| Irf7 | 20 | 0.075 | 0.173 | −0.969 | 1.00E−19 | 2.49E−16 |
| Clic1 | 20 | 0.367 | 0.339 | 0.257 | 1.35E−19 | 3.28E−16 |
| Sepw1 | 20 | 0.835 | 0.942 | −0.289 | 3.77E−19 | 8.92E−16 |
| Cirbp | 20 | 0.334 | 0.499 | −0.454 | 3.82E−19 | 9.01E−16 |
| Skil | 20 | 0.354 | 0.516 | −0.443 | 5.85E−19 | 1.35E−15 |
| Nfkb2 | 20 | 0.123 | 0.229 | −0.681 | 1.60E−18 | 3.54E−15 |
| Tmed2 | 20 | 0.786 | 0.936 | −0.322 | 2.01E−18 | 4.41E−15 |
| Mthfd2 | 20 | 0.139 | 0.262 | −0.725 | 2.09E−18 | 4.56E−15 |
| Trp53i11 | 20 | 0.271 | 0.421 | −0.527 | 2.52E−18 | 5.43E−15 |
| Cybb | 20 | 0.421 | 0.581 | −0.445 | 5.99E−18 | 1.25E−14 |
| Cebpg | 20 | 0.17 | 0.27 | −0.597 | 9.54E−18 | 1.95E−14 |
| Hist1h2ap | 20 | 0.165 | 0.13 | 0.704 | 1.11E−17 | 2.24E−14 |
| Ccng2 | 20 | 0.185 | 0.309 | −0.592 | 1.30E−17 | 2.60E−14 |
| S100a10 | 20 | 0.355 | 0.321 | 0.361 | 1.83E−17 | 3.60E−14 |
| Ctss | 20 | 0.493 | 0.658 | −0.370 | 3.29E−17 | 6.36E−14 |
| Pou2f1 | 20 | 0.103 | 0.207 | −0.733 | 4.59E−17 | 8.75E−14 |
| Dusp4 | 20 | 0.119 | 0.217 | −0.870 | 5.91E−17 | 1.11E−13 |
| Rrad | 20 | 0.165 | 0.261 | −0.972 | 6.96E−17 | 1.29E−13 |
| Sdc1 | 20 | 0.726 | 0.857 | −0.341 | 8.01E−17 | 1.47E−13 |
| Pcbp2 | 20 | 0.585 | 0.758 | −0.388 | 8.14E−17 | 1.49E−13 |
| Junb | 20 | 0.514 | 0.686 | −0.400 | 1.46E−16 | 2.64E−13 |
| Cd48 | 20 | 0.319 | 0.286 | 0.218 | 1.91E−16 | 3.41E−13 |
| Rbm7 | 20 | 0.299 | 0.421 | −0.465 | 1.98E−16 | 3.53E−13 |
| Vim | 20 | 0.254 | 0.215 | 0.525 | 2.46E−16 | 4.37E−13 |
| Zbp1 | 20 | 0.302 | 0.433 | −0.472 | 2.67E−16 | 4.71E−13 |
| Prkcsh | 20 | 0.267 | 0.223 | 0.244 | 3.33E−16 | 5.88E−13 |
| Polr2m | 20 | 0.258 | 0.41 | −0.544 | 5.09E−16 | 8.89E−13 |
| Crem | 20 | 0.186 | 0.303 | −0.549 | 5.43E−16 | 9.43E−13 |
| Serpina3g | 20 | 0.078 | 0.171 | −0.805 | 6.85E−16 | 1.17E−12 |
| Cd79a | 20 | 0.83 | 0.935 | −0.268 | 1.69E−15 | 2.81E−12 |
| Pink1 | 20 | 0.29 | 0.43 | −0.454 | 1.80E−15 | 2.97E−12 |
| Kctd12 | 20 | 0.185 | 0.307 | −0.609 | 1.88E−15 | 3.10E−12 |
| Ypel3 | 20 | 0.394 | 0.562 | −0.391 | 2.91E−15 | 4.72E−12 |
| Elf1 | 20 | 0.292 | 0.404 | −0.469 | 3.95E−15 | 6.32E−12 |
| Smap2 | 20 | 0.254 | 0.397 | −0.512 | 4.54E−15 | 7.20E−12 |
| Jtb | 20 | 0.456 | 0.61 | −0.370 | 4.65E−15 | 7.33E−12 |
| Tram1 | 20 | 0.708 | 0.86 | −0.340 | 5.23E−15 | 8.18E−12 |
| A530040E14Rik | 20 | 0.465 | 0.619 | −0.421 | 6.80E−15 | 1.05E−11 |
| P2ry10 | 20 | 0.108 | 0.206 | −0.645 | 8.23E−15 | 1.27E−11 |
| Pls1 | 20 | 0.14 | 0.243 | −0.573 | 1.11E−14 | 1.70E−11 |
| Srsf9 | 20 | 0.453 | 0.606 | −0.409 | 1.32E−14 | 2.00E−11 |
| Hmgn3 | 20 | 0.373 | 0.502 | −0.399 | 1.82E−14 | 2.72E−11 |
| AC125149.3 | 20 | 0.449 | 0.624 | −0.432 | 1.82E−14 | 2.72E−11 |
| Idh3g | 20 | 0.312 | 0.286 | 0.185 | 2.25E−14 | 3.35E−11 |
| H3f3b | 20 | 0.873 | 0.976 | −0.345 | 2.30E−14 | 3.41E−11 |
| Pqlc3 | 20 | 0.48 | 0.629 | −0.390 | 2.65E−14 | 3.89E−11 |
| Psmc4 | 20 | 0.269 | 0.232 | 0.193 | 2.69E−14 | 3.94E−11 |
| Tyrobp | 20 | 0.144 | 0.252 | −0.592 | 2.99E−14 | 4.36E−11 |
| Hist1h4i | 20 | 0.293 | 0.249 | 0.394 | 3.01E−14 | 4.38E−11 |
| Filip1l | 20 | 0.121 | 0.221 | −0.625 | 3.48E−14 | 5.03E−11 |
| B2m | 20 | 0.965 | 0.984 | −0.278 | 3.56E−14 | 5.13E−11 |
| Irf8 | 20 | 0.087 | 0.173 | −0.833 | 3.80E−14 | 5.44E−11 |
| D16Ertd472e | 20 | 0.142 | 0.24 | −0.595 | 4.87E−14 | 6.90E−11 |
| Dyrk3 | 20 | 0.088 | 0.166 | −0.777 | 5.10E−14 | 7.20E−11 |
| Jund | 20 | 0.875 | 0.978 | −0.243 | 5.84E−14 | 8.21E−11 |
| AW112010 | 20 | 0.287 | 0.443 | −0.396 | 6.42E−14 | 8.98E−11 |
| Jchain | 20 | 0.993 | 0.991 | −0.214 | 9.15E−14 | 1.25E−10 |
| Ormdl3 | 20 | 0.156 | 0.265 | −0.550 | 9.16E−14 | 1.25E−10 |
| Nacc2 | 20 | 0.264 | 0.41 | −0.454 | 1.05E−13 | 1.42E−10 |
| Ldha | 20 | 0.344 | 0.331 | 0.166 | 1.09E−13 | 1.47E−10 |
| Al413582 | 20 | 0.283 | 0.407 | −0.451 | 1.47E−13 | 1.96E−10 |
| Agpat4 | 20 | 0.174 | 0.281 | −0.579 | 1.50E−13 | 2.00E−10 |
| Slc39a7 | 20 | 0.465 | 0.624 | −0.389 | 1.86E−13 | 2.46E−10 |
| Cdv3 | 20 | 0.565 | 0.702 | −0.359 | 2.36E−13 | 3.10E−10 |
| Tgif1 | 20 | 0.109 | 0.199 | −0.659 | 2.49E−13 | 3.27E−10 |
| Bcl10 | 20 | 0.251 | 0.368 | −0.464 | 2.56E−13 | 3.34E−10 |
| Arid5a | 20 | 0.079 | 0.154 | −0.725 | 2.85E−13 | 3.70E−10 |
| Shisa5 | 20 | 0.824 | 0.936 | −0.287 | 2.91E−13 | 3.77E−10 |
| Rheb | 20 | 0.409 | 0.547 | −0.420 | 3.02E−13 | 3.89E−10 |
| Ring1 | 20 | 0.117 | 0.214 | −0.648 | 3.68E−13 | 4.70E−10 |
| Med25 | 20 | 0.126 | 0.19 | −0.712 | 4.62E−13 | 5.83E−10 |
| Aes | 20 | 0.463 | 0.62 | −0.371 | 7.23E−13 | 8.99E−10 |
| Hmgb2 | 20 | 0.272 | 0.307 | 0.336 | 1.06E−12 | 1.30E−09 |
| Tmem184b | 20 | 0.093 | 0.18 | −0.634 | 1.12E−12 | 1.37E−09 |
| Insl6 | 20 | 0.186 | 0.292 | −0.482 | 1.28E−12 | 1.56E−09 |
| Siah2 | 20 | 0.235 | 0.358 | −0.461 | 1.29E−12 | 1.57E−09 |
| Reep3 | 20 | 0.132 | 0.235 | −0.574 | 1.35E−12 | 1.63E−09 |
| Ergic1 | 20 | 0.334 | 0.485 | −0.461 | 1.61E−12 | 1.94E−09 |
| Sumo3 | 20 | 0.206 | 0.327 | −0.531 | 1.95E−12 | 2.33E−09 |
| Azin1 | 20 | 0.33 | 0.473 | −0.426 | 2.08E−12 | 2.47E−09 |
| Psmc2 | 20 | 0.24 | 0.203 | 0.230 | 2.25E−12 | 2.67E−09 |
| Pnrc1 | 20 | 0.607 | 0.767 | −0.334 | 2.26E−12 | 2.67E−09 |
| Rab3d | 20 | 0.162 | 0.267 | −0.517 | 2.34E−12 | 2.75E−09 |
| Cd93 | 20 | 0.137 | 0.24 | −0.527 | 2.87E−12 | 3.35E−09 |
| Gramd3 | 20 | 0.147 | 0.249 | −0.495 | 2.88E−12 | 3.36E−09 |
| Lbh | 20 | 0.177 | 0.283 | −0.502 | 2.97E−12 | 3.45E−09 |
| Arl4a | 20 | 0.124 | 0.221 | −0.585 | 2.98E−12 | 3.46E−09 |
| Ctso | 20 | 0.212 | 0.331 | −0.480 | 3.32E−12 | 3.82E−09 |
| Ier5 | 20 | 0.515 | 0.665 | −0.365 | 3.43E−12 | 3.93E−09 |
| VgIl4 | 20 | 0.142 | 0.238 | −0.587 | 5.08E−12 | 5.73E−09 |
| Map7d1 | 20 | 0.203 | 0.313 | −0.465 | 5.28E−12 | 5.94E−09 |
| Usf1 | 20 | 0.138 | 0.226 | −0.559 | 5.34E−12 | 6.00E−09 |
| Tmem123 | 20 | 0.52 | 0.686 | −0.333 | 5.40E−12 | 6.05E−09 |
| Ctsa | 20 | 0.263 | 0.225 | 0.220 | 6.34E−12 | 7.05E−09 |
| Ginm1 | 20 | 0.181 | 0.281 | −0.479 | 6.54E−12 | 7.26E−09 |
| Tmsb4x | 20 | 0.9 | 0.945 | 0.168 | 7.61E−12 | 8.41E−09 |
| Vmp1 | 20 | 0.346 | 0.481 | −0.432 | 8.85E−12 | 9.68E−09 |
| Dnajb9 | 20 | 0.487 | 0.653 | −0.360 | 9.38E−12 | 1.02E−08 |
| Man1b1 | 20 | 0.334 | 0.478 | −0.422 | 9.99E−12 | 1.08E−08 |
| Rassf3 | 20 | 0.109 | 0.178 | −0.553 | 1.12E−11 | 1.21E−08 |
| Tpst1 | 20 | 0.178 | 0.288 | −0.523 | 1.14E−11 | 1.23E−08 |
| Copb2 | 20 | 0.267 | 0.24 | 0.214 | 1.25E−11 | 1.34E−08 |
| Ube2d3 | 20 | 0.651 | 0.818 | −0.333 | 1.48E−11 | 1.58E−08 |
| Cnn2 | 20 | 0.202 | 0.175 | 0.293 | 1.51E−11 | 1.61E−08 |
| Eif4a2 | 20 | 0.459 | 0.616 | −0.370 | 1.53E−11 | 1.62E−08 |
| Tmem192 | 20 | 0.249 | 0.372 | −0.444 | 1.96E−11 | 2.07E−08 |
| Sec11c | 20 | 0.898 | 0.972 | −0.233 | 2.74E−11 | 2.86E−08 |
| Casp4 | 20 | 0.202 | 0.309 | −0.472 | 2.87E−11 | 2.97E−08 |
| St6gal1 | 20 | 0.583 | 0.738 | −0.339 | 3.02E−11 | 3.11E−08 |
| Kdm2b | 20 | 0.1 | 0.186 | −0.646 | 3.10E−11 | 3.19E−08 |
| Ckb | 20 | 0.179 | 0.141 | 0.331 | 3.72E−11 | 3.81E−08 |
| Scand1 | 20 | 0.513 | 0.651 | −0.340 | 4.15E−11 | 4.22E−08 |
| Rab28 | 20 | 0.124 | 0.213 | −0.548 | 4.19E−11 | 4.26E−08 |
| Bmi1 | 20 | 0.095 | 0.179 | −0.579 | 4.52E−11 | 4.57E−08 |
| Cers2 | 20 | 0.319 | 0.443 | −0.412 | 4.80E−11 | 4.82E−08 |
| Sdha | 20 | 0.255 | 0.22 | 0.151 | 5.60E−11 | 5.59E−08 |
| Vapa | 20 | 0.403 | 0.547 | −0.378 | 6.08E−11 | 6.05E−08 |
| Stoml1 | 20 | 0.091 | 0.163 | −0.634 | 6.48E−11 | 6.43E−08 |
| Idnk | 20 | 0.277 | 0.393 | −0.387 | 7.20E−11 | 7.11E−08 |
| Tmem219 | 20 | 0.309 | 0.44 | −0.399 | 8.75E−11 | 8.51E−08 |
| Commd3 | 20 | 0.317 | 0.448 | −0.400 | 9.50E−11 | 9.18E−08 |
| Kif5b | 20 | 0.356 | 0.5 | −0.423 | 1.17E−10 | 1.12E−07 |
| Klhdc1 | 20 | 0.083 | 0.154 | −0.601 | 1.81E−10 | 1.69E−07 |
| Pml | 20 | 0.184 | 0.284 | −0.515 | 1.85E−10 | 1.72E−07 |
| Tcf4 | 20 | 0.42 | 0.551 | −0.385 | 1.87E−10 | 1.74E−07 |
| Tram2 | 20 | 0.287 | 0.426 | −0.395 | 2.12E−10 | 1.95E−07 |
| Ppp1r11 | 20 | 0.404 | 0.535 | −0.368 | 2.34E−10 | 2.14E−07 |
| Smim19 | 20 | 0.123 | 0.201 | −0.511 | 2.40E−10 | 2.19E−07 |
| Limd1 | 20 | 0.164 | 0.256 | −0.529 | 3.52E−10 | 3.13E−07 |
| Cd200 | 20 | 0.428 | 0.595 | −0.279 | 3.54E−10 | 3.14E−07 |
| PigP | 20 | 0.142 | 0.217 | −0.492 | 3.88E−10 | 3.38E−07 |
| A630072M18Rik | 20 | 0.085 | 0.16 | −0.686 | 4.35E−10 | 3.77E−07 |
| Pabpc1 | 20 | 0.708 | 0.867 | −0.300 | 5.39E−10 | 4.66E−07 |
| Orai1 | 20 | 0.151 | 0.233 | −0.466 | 8.79E−10 | 7.39E−07 |
| Spg21 | 20 | 0.176 | 0.276 | −0.489 | 9.10E−10 | 7.62E−07 |
| Actb | 20 | 0.953 | 0.968 | 0.250 | 9.34E−10 | 7.80E−07 |
| Ddrgk1 | 20 | 0.533 | 0.672 | −0.321 | 9.80E−10 | 8.16E−07 |
| Cebpb | 20 | 0.27 | 0.382 | −0.374 | 1.06E−09 | 8.76E−07 |
| Hiat1 | 20 | 0.132 | 0.204 | −0.490 | 1.06E−09 | 8.76E−07 |
| Ubxn6 | 20 | 0.201 | 0.296 | −0.426 | 1.07E−09 | 8.78E−07 |
| Fam214a | 20 | 0.228 | 0.346 | −0.415 | 1.10E−09 | 9.05E−07 |
| Stub1 | 20 | 0.22 | 0.33 | −0.454 | 1.33E−09 | 1.07E−06 |
| Chtf8 | 20 | 0.09 | 0.154 | −0.560 | 1.41E−09 | 1.13E−06 |
| Fcer1g | 20 | 0.293 | 0.407 | −0.335 | 1.51E−09 | 1.21E−06 |
| Plbd2 | 20 | 0.156 | 0.251 | −0.479 | 1.69E−09 | 1.34E−06 |
| Drap1 | 20 | 0.285 | 0.392 | −0.390 | 1.74E−09 | 1.38E−06 |
| 1500011B03Rik | 20 | 0.166 | 0.257 | −0.506 | 1.75E−09 | 1.38E−06 |
| Gpx4 | 20 | 0.626 | 0.759 | −0.299 | 1.94E−09 | 1.52E−06 |
| Gng5 | 20 | 0.572 | 0.709 | −0.307 | 1.94E−09 | 1.52E−06 |
| Bcl2l11 | 20 | 0.269 | 0.369 | −0.489 | 1.96E−09 | 1.53E−06 |
| Cflar | 20 | 0.222 | 0.328 | −0.458 | 1.98E−09 | 1.55E−06 |
| Ddx39 | 20 | 0.169 | 0.142 | 0.237 | 2.16E−09 | 1.68E−06 |
| Tspan13 | 20 | 0.744 | 0.886 | −0.288 | 2.17E−09 | 1.68E−06 |
| Ppp2r5a | 20 | 0.332 | 0.453 | −0.392 | 2.37E−09 | 1.84E−06 |
| Lrrfip2 | 20 | 0.091 | 0.159 | −0.580 | 2.38E−09 | 1.84E−06 |
| EIl2 | 20 | 0.543 | 0.697 | −0.344 | 2.41E−09 | 1.85E−06 |
| Btg3 | 20 | 0.251 | 0.358 | −0.417 | 2.42E−09 | 1.86E−06 |
| Scamp3 | 20 | 0.126 | 0.209 | −0.532 | 2.46E−09 | 1.89E−06 |
| Ptp4a1 | 20 | 0.208 | 0.313 | −0.448 | 2.76E−09 | 2.10E−06 |
| Mettl1 | 20 | 0.278 | 0.383 | −0.404 | 2.85E−09 | 2.16E−06 |
| Relb | 20 | 0.127 | 0.203 | −0.501 | 2.90E−09 | 2.19E−06 |
| Mxd1 | 20 | 0.126 | 0.207 | −0.515 | 3.08E−09 | 2.31E−06 |
| Bcl2 | 20 | 0.221 | 0.336 | −0.342 | 3.47E−09 | 2.58E−06 |
| Akirin1 | 20 | 0.293 | 0.408 | −0.379 | 3.60E−09 | 2.67E−06 |
| 4930453N24Rik | 20 | 0.111 | 0.159 | −0.464 | 3.66E−09 | 2.70E−06 |
| Nxf1 | 20 | 0.286 | 0.401 | −0.389 | 4.18E−09 | 3.05E−06 |
| Fam174a | 20 | 0.181 | 0.274 | −0.391 | 4.23E−09 | 3.07E−06 |
| Rad23a | 20 | 0.147 | 0.245 | −0.460 | 4.23E−09 | 3.07E−06 |
| Ptp4a2 | 20 | 0.666 | 0.82 | −0.304 | 4.54E−09 | 3.29E−06 |
| Rad23b | 20 | 0.353 | 0.486 | −0.376 | 4.99E−09 | 3.60E−06 |
| Arrdc3 | 20 | 0.136 | 0.226 | −0.504 | 5.05E−09 | 3.63E−06 |
| Tsg101 | 20 | 0.193 | 0.297 | −0.456 | 5.65E−09 | 4.04E−06 |
| Blmh | 20 | 0.177 | 0.148 | 0.173 | 6.02E−09 | 4.29E−06 |
| H2-T22 | 20 | 0.165 | 0.249 | −0.450 | 6.32E−09 | 4.49E−06 |
| Uqcc2 | 20 | 0.365 | 0.504 | −0.382 | 6.66E−09 | 4.73E−06 |
| Tmem251 | 20 | 0.139 | 0.225 | −0.487 | 6.79E−09 | 4.80E−06 |
| Tor2a | 20 | 0.313 | 0.425 | −0.372 | 6.85E−09 | 4.84E−06 |
| Sel1l | 20 | 0.473 | 0.608 | −0.346 | 7.02E−09 | 4.95E−06 |
| Tmc6 | 20 | 0.169 | 0.247 | −0.401 | 7.42E−09 | 5.22E−06 |
| Psip1 | 20 | 0.341 | 0.469 | −0.395 | 7.44E−09 | 5.22E−06 |
| Btg1 | 20 | 0.208 | 0.313 | −0.369 | 7.53E−09 | 5.28E−06 |
| Gimap1 | 20 | 0.649 | 0.789 | −0.305 | 7.54E−09 | 5.28E−06 |
| Tuba1b | 20 | 0.209 | 0.225 | 0.341 | 8.54E−09 | 5.93E−06 |
| Cib2 | 20 | 0.126 | 0.195 | −0.474 | 9.05E−09 | 6.24E−06 |
| Tapbpl | 20 | 0.276 | 0.4 | −0.376 | 9.07E−09 | 6.25E−06 |
| Smarca2 | 20 | 0.113 | 0.187 | −0.560 | 9.48E−09 | 6.51E−06 |
| Wnk1 | 20 | 0.26 | 0.357 | −0.421 | 9.80E−09 | 6.73E−06 |
| H2afj | 20 | 0.583 | 0.73 | −0.310 | 1.01E−08 | 6.88E−06 |
| Ikzf3 | 20 | 0.167 | 0.263 | −0.464 | 1.41E−08 | 9.43E−06 |
| Zc3h14 | 20 | 0.133 | 0.218 | −0.523 | 1.51E−08 | 1.01E−05 |
| Lrp10 | 20 | 0.336 | 0.461 | −0.368 | 1.73E−08 | 1.15E−05 |
| Foxp1 | 20 | 0.218 | 0.313 | −0.425 | 1.98E−08 | 1.30E−05 |
| Rap1b | 20 | 0.479 | 0.617 | −0.343 | 2.06E−08 | 1.34E−05 |
| Nfkbie | 20 | 0.089 | 0.159 | −0.540 | 2.07E−08 | 1.35E−05 |
| Pkig | 20 | 0.578 | 0.733 | −0.314 | 2.08E−08 | 1.36E−05 |
| Laptm5 | 20 | 0.422 | 0.56 | −0.343 | 2.13E−08 | 1.39E−05 |
| Mast4 | 20 | 0.129 | 0.2 | −0.492 | 2.15E−08 | 1.40E−05 |
| Cd83 | 20 | 0.106 | 0.184 | −0.492 | 2.47E−08 | 1.59E−05 |
| Dnajc7 | 20 | 0.582 | 0.713 | −0.312 | 2.69E−08 | 1.72E−05 |
| Gnl3 | 20 | 0.142 | 0.237 | −0.503 | 2.92E−08 | 1.85E−05 |
| Nudt18 | 20 | 0.252 | 0.35 | −0.398 | 2.96E−08 | 1.87E−05 |
| Rftn1 | 20 | 0.378 | 0.5 | −0.347 | 3.03E−08 | 1.92E−05 |
| 1810043H04Rik | 20 | 0.265 | 0.385 | −0.384 | 3.09E−08 | 1.95E−05 |
| Desi1 | 20 | 0.487 | 0.62 | −0.330 | 3.28E−08 | 2.06E−05 |
| Ppp1r2 | 20 | 0.211 | 0.311 | −0.443 | 3.31E−08 | 2.07E−05 |
| Napsa | 20 | 0.202 | 0.171 | 0.207 | 3.51E−08 | 2.19E−05 |
| Slc3a2 | 20 | 0.528 | 0.67 | −0.297 | 3.59E−08 | 2.22E−05 |
| Pfdn5 | 20 | 0.784 | 0.906 | −0.267 | 3.67E−08 | 2.28E−05 |
| Scarb2 | 20 | 0.189 | 0.284 | −0.412 | 3.89E−08 | 2.39E−05 |
| Sh3glb1 | 20 | 0.483 | 0.621 | −0.334 | 4.12E−08 | 2.52E−05 |
| Ddit3 | 20 | 0.181 | 0.275 | −0.432 | 4.32E−08 | 2.63E−05 |
| Cars | 20 | 0.113 | 0.187 | −0.508 | 4.65E−08 | 2.82E−05 |
| 4933434E20Rik | 20 | 0.35 | 0.473 | −0.366 | 4.78E−08 | 2.89E−05 |
| Pcif1 | 20 | 0.248 | 0.358 | −0.379 | 4.80E−08 | 2.89E−05 |
| Tpst2 | 20 | 0.51 | 0.654 | −0.309 | 5.01E−08 | 3.01E−05 |
| Rab10 | 20 | 0.31 | 0.438 | −0.364 | 5.12E−08 | 3.07E−05 |
| Edem2 | 20 | 0.813 | 0.935 | −0.257 | 5.31E−08 | 3.18E−05 |
| Pofut2 | 20 | 0.137 | 0.216 | −0.467 | 5.38E−08 | 3.22E−05 |
| Mapkapk2 | 20 | 0.142 | 0.218 | −0.459 | 5.38E−08 | 3.22E−05 |
| Brcc3 | 20 | 0.163 | 0.138 | 0.202 | 5.90E−08 | 3.49E−05 |
| Pex16 | 20 | 0.161 | 0.242 | −0.386 | 6.63E−08 | 3.90E−05 |
| Tpd52 | 20 | 0.128 | 0.207 | −0.463 | 7.96E−08 | 4.60E−05 |
| Anapc5 | 20 | 0.469 | 0.621 | −0.357 | 8.19E−08 | 4.71E−05 |
| Smc4 | 20 | 0.186 | 0.169 | 0.183 | 8.60E−08 | 4.94E−05 |
| Arsg | 20 | 0.092 | 0.153 | −0.506 | 1.25E−07 | 7.00E−05 |
| Ltb | 20 | 0.183 | 0.259 | −0.485 | 1.34E−07 | 7.50E−05 |
| Ap3s1 | 20 | 0.402 | 0.505 | −0.304 | 1.36E−07 | 7.60E−05 |
| Hmg20b | 20 | 0.123 | 0.192 | −0.504 | 1.50E−07 | 8.34E−05 |
| Rnf19b | 20 | 0.172 | 0.264 | −0.414 | 1.52E−07 | 8.39E−05 |
| Ptpra | 20 | 0.097 | 0.161 | −0.511 | 1.55E−07 | 8.58E−05 |
| Tm2d1 | 20 | 0.195 | 0.291 | −0.423 | 1.65E−07 | 9.08E−05 |
| Amfr | 20 | 0.276 | 0.382 | −0.349 | 1.66E−07 | 9.10E−05 |
| Mgat2 | 20 | 0.54 | 0.669 | −0.315 | 1.81E−07 | 9.86E−05 |
| Ccz1 | 20 | 0.219 | 0.303 | −0.410 | 1.87E−07 | 1.02E−04 |
| Btf3l4 | 20 | 0.18 | 0.271 | −0.397 | 1.92E−07 | 1.04E−04 |
| Dnajc19 | 20 | 0.386 | 0.535 | −0.346 | 1.93E−07 | 1.05E−04 |
| Cyth2 | 20 | 0.161 | 0.242 | −0.430 | 2.05E−07 | 1.11E−04 |
| Ggta1 | 20 | 0.11 | 0.178 | −0.538 | 2.15E−07 | 1.15E−04 |
| Fam177a | 20 | 0.103 | 0.173 | −0.530 | 2.43E−07 | 1.29E−04 |
| Bet1l | 20 | 0.242 | 0.322 | −0.349 | 2.48E−07 | 1.31E−04 |
| Cdipt | 20 | 0.238 | 0.34 | −0.416 | 2.56E−07 | 1.35E−04 |
| Zfp560 | 20 | 0.13 | 0.198 | −0.422 | 2.67E−07 | 1.41E−04 |
| Wipi1 | 20 | 0.23 | 0.329 | −0.392 | 2.72E−07 | 1.43E−04 |
| Ninj1 | 20 | 0.273 | 0.387 | −0.364 | 2.77E−07 | 1.45E−04 |
| Ndufaf4 | 20 | 0.092 | 0.16 | −0.555 | 2.86E−07 | 1.50E−04 |
| Rapgef1 | 20 | 0.133 | 0.207 | −0.452 | 2.92E−07 | 1.53E−04 |
| Ddx5 | 20 | 0.736 | 0.859 | −0.266 | 2.94E−07 | 1.53E−04 |
| Ddx54 | 20 | 0.233 | 0.334 | −0.388 | 3.04E−07 | 1.58E−04 |
| Ube2e3 | 20 | 0.264 | 0.374 | −0.350 | 3.09E−07 | 1.60E−04 |
| Eif5 | 20 | 0.587 | 0.732 | −0.308 | 3.23E−07 | 1.67E−04 |
| Hes1 | 20 | 0.2 | 0.274 | −0.641 | 3.29E−07 | 1.70E−04 |
| Tusc2 | 20 | 0.134 | 0.207 | −0.465 | 3.31E−07 | 1.71E−04 |
| Tiparp | 20 | 0.249 | 0.358 | −0.387 | 3.52E−07 | 1.80E−04 |
| Gbp4 | 20 | 0.093 | 0.158 | −0.519 | 3.90E−07 | 1.99E−04 |
| Ankrd12 | 20 | 0.256 | 0.349 | −0.361 | 4.18E−07 | 2.12E−04 |
| Fastk | 20 | 0.177 | 0.262 | −0.425 | 4.21E−07 | 2.13E−04 |
| Atg101 | 20 | 0.15 | 0.222 | −0.439 | 4.38E−07 | 2.21E−04 |
| Mdm2 | 20 | 0.214 | 0.303 | −0.414 | 4.46E−07 | 2.24E−04 |
| Tgfb1 | 20 | 0.228 | 0.308 | −0.387 | 4.47E−07 | 2.25E−04 |
| Bri3 | 20 | 0.211 | 0.31 | −0.379 | 4.64E−07 | 2.33E−04 |
| Stard5 | 20 | 0.16 | 0.254 | −0.409 | 4.81E−07 | 2.41E−04 |
| Irf4 | 20 | 0.348 | 0.479 | −0.375 | 5.16E−07 | 2.57E−04 |
| Upf3b | 20 | 0.174 | 0.221 | −0.309 | 5.18E−07 | 2.57E−04 |
| Ubald1 | 20 | 0.17 | 0.246 | −0.432 | 5.35E−07 | 2.65E−04 |
| Rab3a | 20 | 0.115 | 0.186 | −0.511 | 5.46E−07 | 2.70E−04 |
| Pck2 | 20 | 0.266 | 0.344 | −0.325 | 5.60E−07 | 2.76E−04 |
| Grina | 20 | 0.337 | 0.444 | −0.312 | 5.64E−07 | 2.78E−04 |
| Dph3 | 20 | 0.356 | 0.479 | −0.351 | 5.91E−07 | 2.90E−04 |
| Eif4a3 | 20 | 0.221 | 0.203 | 0.151 | 5.94E−07 | 2.92E−04 |
| Zc3h15 | 20 | 0.3 | 0.389 | −0.352 | 5.98E−07 | 2.93E−04 |
| Ppp3ca | 20 | 0.262 | 0.364 | −0.363 | 6.03E−07 | 2.95E−04 |
| Clptm1l | 20 | 0.651 | 0.788 | −0.293 | 7.17E−07 | 3.44E−04 |
| Dera | 20 | 0.179 | 0.16 | 0.158 | 7.18E−07 | 3.44E−04 |
| St8sia6 | 20 | 0.247 | 0.344 | −0.382 | 7.19E−07 | 3.45E−04 |
| Cited2 | 20 | 0.542 | 0.611 | 0.167 | 7.22E−07 | 3.45E−04 |
| Sar1a | 20 | 0.546 | 0.695 | −0.306 | 7.28E−07 | 3.48E−04 |
| Wipf1 | 20 | 0.121 | 0.199 | −0.474 | 7.39E−07 | 3.53E−04 |
| 4930523C07Rik | 20 | 0.307 | 0.411 | −0.393 | 7.45E−07 | 3.55E−04 |
| Mta2 | 20 | 0.256 | 0.34 | −0.367 | 8.25E−07 | 3.90E−04 |
| Ptpn6 | 20 | 0.268 | 0.375 | −0.376 | 8.37E−07 | 3.95E−04 |
| Pgrmc1 | 20 | 0.205 | 0.285 | −0.376 | 8.69E−07 | 4.08 E−04 |
| Fndc3a | 20 | 0.324 | 0.425 | −0.331 | 8.70E−07 | 4.08 E−04 |
| Gtf3c6 | 20 | 0.181 | 0.166 | 0.189 | 9.88E−07 | 4.60E−04 |
| Ccs | 20 | 0.158 | 0.135 | 0.190 | 9.90E−07 | 4.61E−04 |
| Lrrn3 | 20 | 0.099 | 0.156 | −0.521 | 1.01E−06 | 4.66E−04 |
| Tcf3 | 20 | 0.326 | 0.404 | −0.300 | 1.10E−06 | 5.05E−04 |
| Mob1a | 20 | 0.1 | 0.159 | −0.493 | 1.10E−06 | 5.07E−04 |
| Setd3 | 20 | 0.132 | 0.207 | −0.408 | 1.17E−06 | 5.33E−04 |
| Sdc4 | 20 | 0.423 | 0.533 | −0.265 | 1.17E−06 | 5.33E−04 |
| Rp9 | 20 | 0.342 | 0.455 | −0.351 | 1.17E−06 | 5.33E−04 |
| Tomm20 | 20 | 0.408 | 0.529 | −0.332 | 1.17E−06 | 5.33E−04 |
| Vamp3 | 20 | 0.253 | 0.341 | −0.366 | 1.19E−06 | 5.44E−04 |
| Herpud2 | 20 | 0.121 | 0.177 | −0.398 | 1.20E−06 | 5.45E−04 |
| Dctn3 | 20 | 0.349 | 0.404 | −0.287 | 1.27E−06 | 5.77E−04 |
| Pitpnb | 20 | 0.101 | 0.161 | −0.517 | 1.29E−06 | 5.85E−04 |
| Csnk1a1 | 20 | 0.34 | 0.437 | −0.338 | 1.30E−06 | 5.89E−04 |
| Dynlt3 | 20 | 0.261 | 0.348 | −0.342 | 1.34E−06 | 6.01E−04 |
| Arpc5l | 20 | 0.444 | 0.588 | −0.332 | 1.35E−06 | 6.05E−04 |
| Atp6v1d | 20 | 0.2 | 0.284 | −0.371 | 1.37E−06 | 6.14E−04 |
| Ccpg1 | 20 | 0.33 | 0.426 | −0.263 | 1.40E−06 | 6.27E−04 |
| Cytip | 20 | 0.619 | 0.753 | −0.297 | 1.54E−06 | 6.80E−04 |
| Tmem147 | 20 | 0.413 | 0.54 | −0.323 | 1.61E−06 | 7.11E−04 |
| Isyna1 | 20 | 0.165 | 0.149 | 0.169 | 1.62E−06 | 7.12E−04 |
| Bhlha15 | 20 | 0.213 | 0.309 | −0.404 | 1.64E−06 | 7.19E−04 |
| A930033H14Rik | 20 | 0.104 | 0.165 | −0.518 | 1.68E−06 | 7.39E−04 |
| Vimp | 20 | 0.72 | 0.846 | −0.262 | 1.71E−06 | 7.50E−04 |
| Asns | 20 | 0.096 | 0.165 | −0.481 | 1.74E−06 | 7.62E−04 |
| Sp140 | 20 | 0.306 | 0.399 | −0.342 | 1.75E−06 | 7.65E−04 |
| Sumo1 | 20 | 0.442 | 0.58 | −0.315 | 1.78E−06 | 7.76E−04 |
| Toporsos | 20 | 0.151 | 0.229 | −0.459 | 1.78E−06 | 7.76E−04 |
| March5 | 20 | 0.273 | 0.369 | −0.354 | 1.88E−06 | 8.13E−04 |
| Srpr | 20 | 0.446 | 0.585 | −0.309 | 1.90E−06 | 8.21E−04 |
| Inpp1 | 20 | 0.119 | 0.186 | −0.447 | 1.91E−06 | 8.25E−04 |
| Emc6 | 20 | 0.288 | 0.383 | −0.328 | 2.02E−06 | 8.62E−04 |
| Spint2 | 20 | 0.64 | 0.764 | −0.248 | 2.05E−06 | 8.76E−04 |
| Fbxw2 | 20 | 0.183 | 0.261 | −0.418 | 2.15E−06 | 9.16E−04 |
| Atp6v0a1 | 20 | 0.253 | 0.352 | −0.351 | 2.16E−06 | 9.16E−04 |
| Myl12a | 20 | 0.505 | 0.637 | −0.314 | 2.26E−06 | 9.54E−04 |
| Unc50 | 20 | 0.265 | 0.37 | −0.356 | 2.31E−06 | 9.75E−04 |
| Akap9 | 20 | 0.116 | 0.186 | −0.471 | 2.33E−06 | 9.83E−04 |
| Tmem248 | 20 | 0.615 | 0.767 | −0.292 | 2.35E−06 | 9.91E−04 |
| D8Ertd738e | 20 | 0.503 | 0.657 | −0.312 | 2.41E−06 | 1.01E−03 |
| Stx7 | 20 | 0.132 | 0.205 | −0.465 | 2.43E−06 | 1.02E−03 |
| Rela | 20 | 0.113 | 0.179 | −0.447 | 2.45E−06 | 1.03E−03 |
| Smim20 | 20 | 0.133 | 0.194 | −0.424 | 2.49E−06 | 1.04E−03 |
| Sod2 | 20 | 0.363 | 0.468 | −0.313 | 2.50E−06 | 1.04E−03 |
| Zfand5 | 20 | 0.23 | 0.328 | −0.406 | 2.51E−06 | 1.05E−03 |
| Atat1 | 20 | 0.397 | 0.525 | −0.325 | 2.63E−06 | 1.09E−03 |
| Vars | 20 | 0.394 | 0.526 | −0.346 | 2.72E−06 | 1.12E−03 |
| Lrrc8d | 20 | 0.14 | 0.21 | −0.453 | 2.79E−06 | 1.15E−03 |
| Ube2g2 | 20 | 0.14 | 0.214 | −0.431 | 2.83E−06 | 1.16E−03 |
| Ap4s1 | 20 | 0.185 | 0.245 | −0.357 | 2.91E−06 | 1.19E−03 |
| Arl2bp | 20 | 0.216 | 0.295 | −0.341 | 2.92E−06 | 1.19E−03 |
| Ralbp1 | 20 | 0.332 | 0.445 | −0.372 | 3.05E−06 | 1.23E−03 |
| Dtx3 | 20 | 0.114 | 0.174 | −0.431 | 3.07E−06 | 1.24E−03 |
| Golga4 | 20 | 0.175 | 0.243 | −0.373 | 3.13E−06 | 1.26E−03 |
| Mia3 | 20 | 0.188 | 0.267 | −0.399 | 3.14E−06 | 1.27E−03 |
| Birc3 | 20 | 0.153 | 0.23 | −0.368 | 3.14E−06 | 1.27E−03 |
| Wbp11 | 20 | 0.124 | 0.191 | −0.517 | 3.15E−06 | 1.27E−03 |
| Atf6 | 20 | 0.197 | 0.278 | −0.391 | 3.28E−06 | 1.32E−03 |
| Jun | 20 | 0.733 | 0.757 | −0.151 | 3.32E−06 | 1.33E−03 |
| Tmem230 | 20 | 0.117 | 0.181 | −0.463 | 3.34E−06 | 1.34E−03 |
| Ccdc117 | 20 | 0.21 | 0.279 | −0.326 | 3.50E−06 | 1.39E−03 |
| Cggbp1 | 20 | 0.171 | 0.251 | −0.432 | 3.56E−06 | 1.42E−03 |
| Dock10 | 20 | 0.15 | 0.213 | −0.387 | 3.59E−06 | 1.43E−03 |
| Manea | 20 | 0.26 | 0.324 | −0.280 | 3.65E−06 | 1.45E−03 |
| Rab7 | 20 | 0.362 | 0.457 | −0.290 | 3.80E−06 | 1.50E−03 |
| Etf1 | 20 | 0.261 | 0.354 | −0.384 | 4.27E−06 | 1.66E−03 |
| Copz2 | 20 | 0.115 | 0.158 | −0.399 | 4.38E−06 | 1.70E−03 |
| Rnf10 | 20 | 0.282 | 0.382 | −0.376 | 4.97E−06 | 1.92E−03 |
| Fdx1l | 20 | 0.115 | 0.182 | −0.441 | 5.02E−06 | 1.93E−03 |
| Golt1b | 20 | 0.18 | 0.259 | −0.410 | 5.07E−06 | 1.95E−03 |
| Fcrla | 20 | 0.252 | 0.361 | −0.333 | 5.29E−06 | 2.02E−03 |
| Plekha2 | 20 | 0.216 | 0.305 | −0.386 | 5.35E−06 | 2.04E−03 |
| Hbp1 | 20 | 0.2 | 0.271 | −0.332 | 5.38E−06 | 2.05E−03 |
| Emc2 | 20 | 0.167 | 0.15 | 0.151 | 5.56E−06 | 2.11E−03 |
| Ryr1 | 20 | 0.23 | 0.32 | −0.341 | 5.72E−06 | 2.17E−03 |
| Al467606 | 20 | 0.103 | 0.164 | −0.523 | 5.77E−06 | 2.19E−03 |
| Tuba1a | 20 | 0.291 | 0.41 | −0.449 | 5.90E−06 | 2.23E−03 |
| Rab5a | 20 | 0.284 | 0.366 | −0.294 | 6.49E−06 | 2.43E−03 |
| Nudt9 | 20 | 0.179 | 0.252 | −0.386 | 6.54E−06 | 2.44E−03 |
| Taz | 20 | 0.138 | 0.193 | −0.366 | 6.60E−06 | 2.46E−03 |
| Oser1 | 20 | 0.234 | 0.317 | −0.348 | 6.73E−06 | 2.51E−03 |
| 2300009A05Rik | 20 | 0.129 | 0.2 | −0.421 | 6.99E−06 | 2.59E−03 |
| Gigyf2 | 20 | 0.109 | 0.173 | −0.503 | 7.09E−06 | 2.63E−03 |
| Tmed3 | 20 | 0.344 | 0.437 | −0.304 | 7.37E−06 | 2.72E−03 |
| Mkrn1 | 20 | 0.391 | 0.477 | −0.262 | 7.63E−06 | 2.80E−03 |
| Bbs9 | 20 | 0.206 | 0.29 | −0.360 | 7.79E−06 | 2.86E−03 |
| Ddx24 | 20 | 0.193 | 0.273 | −0.385 | 7.92E−06 | 2.90E−03 |
| Hint2 | 20 | 0.165 | 0.238 | −0.341 | 8.36E−06 | 3.04E−03 |
| Tnfaip3 | 20 | 0.194 | 0.27 | −0.361 | 8.39E−06 | 3.04E−03 |
| Vamp8 | 20 | 0.502 | 0.644 | −0.277 | 8.42E−06 | 3.05E−03 |
| Gem | 20 | 0.198 | 0.181 | 0.200 | 8.67E−06 | 3.14E−03 |
| Guk1 | 20 | 0.211 | 0.302 | −0.346 | 8.88E−06 | 3.20E−03 |
| Slain1 | 20 | 0.166 | 0.233 | −0.350 | 9.30E−06 | 3.34E−03 |
| Ccnd2 | 20 | 0.404 | 0.526 | −0.335 | 9.66E−06 | 3.46E−03 |
| Rbm3 | 20 | 0.773 | 0.888 | −0.244 | 9.83E−06 | 3.50E−03 |
| Arpc1a | 20 | 0.335 | 0.443 | −0.330 | 9.90E−06 | 3.52E−03 |
| Fam168b | 20 | 0.111 | 0.173 | −0.481 | 9.97E−06 | 3.55E−03 |
| Gosr2 | 20 | 0.265 | 0.368 | −0.384 | 1.02E−05 | 3.62E−03 |
| Nudc | 20 | 0.235 | 0.336 | −0.392 | 1.03E−05 | 3.64E−03 |
| 0610012G03Rik | 20 | 0.154 | 0.231 | −0.418 | 1.04E−05 | 3.69E−03 |
| Rnf11 | 20 | 0.11 | 0.168 | −0.386 | 1.07E−05 | 3.79E−03 |
| BC004004 | 20 | 0.438 | 0.576 | −0.318 | 1.08E−05 | 3.79E−03 |
| Tspan31 | 20 | 0.115 | 0.187 | −0.449 | 1.09E−05 | 3.85E−03 |
| Qrich1 | 20 | 0.167 | 0.242 | −0.407 | 1.15E−05 | 4.04E−03 |
| Ccm2 | 20 | 0.227 | 0.323 | −0.336 | 1.15E−05 | 4.04E−03 |
| Bhlhe41 | 20 | 0.247 | 0.335 | −0.419 | 1.17E−05 | 4.09E−03 |
| Usf2 | 20 | 0.135 | 0.203 | −0.405 | 1.17E−05 | 4.10E−03 |
| Chrac1 | 20 | 0.207 | 0.29 | −0.380 | 1.24E−05 | 4.30E−03 |
| Arpc2 | 20 | 0.622 | 0.781 | −0.295 | 1.28E−05 | 4.44E−03 |
| Cnot11 | 20 | 0.108 | 0.157 | −0.413 | 1.40E−05 | 4.80E−03 |
| Gorasp2 | 20 | 0.446 | 0.569 | −0.313 | 1.41E−05 | 4.83E−03 |
| Kcnq1ot1 | 20 | 0.209 | 0.287 | −0.385 | 1.43E−05 | 4.88E−03 |
| Srpk2 | 20 | 0.203 | 0.282 | −0.378 | 1.45E−05 | 4.95E−03 |
| Rfk | 20 | 0.131 | 0.206 | −0.441 | 1.53E−05 | 5.18E−03 |
| Rab24 | 20 | 0.154 | 0.234 | −0.432 | 1.54E−05 | 5.20E−03 |
| Ndufb4 | 20 | 0.409 | 0.522 | −0.306 | 1.61E−05 | 5.43E−03 |
| Brd2 | 20 | 0.296 | 0.387 | −0.323 | 1.69E−05 | 5.66E−03 |
| Adssl1 | 20 | 0.125 | 0.196 | −0.370 | 1.74E−05 | 5.78E−03 |
| Pnrc2 | 20 | 0.157 | 0.237 | −0.376 | 1.77E−05 | 5.87E−03 |
| Evi2a | 20 | 0.473 | 0.593 | −0.315 | 1.77E−05 | 5.88E−03 |
| Gng12 | 20 | 0.157 | 0.228 | −0.365 | 1.84E−05 | 6.07E−03 |
| Chmp1a | 20 | 0.102 | 0.159 | −0.497 | 1.84E−05 | 6.07E−03 |
| Smchd1 | 20 | 0.289 | 0.38 | −0.344 | 1.87E−05 | 6.16E−03 |
| Ccdc88a | 20 | 0.255 | 0.337 | −0.334 | 1.99E−05 | 6.49E−03 |
| Gna13 | 20 | 0.152 | 0.213 | −0.430 | 2.00E−05 | 6.52E−03 |
| Lyn | 20 | 0.119 | 0.173 | −0.419 | 2.08E−05 | 6.77E−03 |
| St8sia4 | 20 | 0.25 | 0.345 | −0.376 | 2.10E−05 | 6.82E−03 |
| Psmg4 | 20 | 0.195 | 0.275 | −0.388 | 2.13E−05 | 6.89E−03 |
| Tgoln1 | 20 | 0.229 | 0.324 | −0.362 | 2.15E−05 | 6.95E−03 |
| Fam46c | 20 | 0.658 | 0.79 | −0.265 | 2.16E−05 | 6.98E−03 |
| Eif4g2 | 20 | 0.537 | 0.677 | −0.295 | 2.18E−05 | 7.03E−03 |
| C130026l21Rik | 20 | 0.151 | 0.213 | −0.399 | 2.19E−05 | 7.04E−03 |
| Mat2b | 20 | 0.252 | 0.344 | −0.338 | 2.20E−05 | 7.09E−03 |
| Gde1 | 20 | 0.181 | 0.266 | −0.387 | 2.32E−05 | 7.41E−03 |
| Atp6v1f | 20 | 0.587 | 0.727 | −0.290 | 2.33E−05 | 7.45E−03 |
| Tob1 | 20 | 0.165 | 0.231 | −0.381 | 2.37E−05 | 7.55E−03 |
| Bcl7c | 20 | 0.256 | 0.34 | −0.338 | 2.44E−05 | 7.75E−03 |
| B4galnt1 | 20 | 0.185 | 0.258 | −0.365 | 2.44E−05 | 7.76E−03 |
| Nudcd3 | 20 | 0.138 | 0.186 | −0.361 | 2.45E−05 | 7.77E−03 |
| Swap70 | 20 | 0.121 | 0.167 | −0.377 | 2.51E−05 | 7.95E−03 |
| Arf6 | 20 | 0.517 | 0.643 | −0.286 | 2.53E−05 | 8.00E−03 |
| Dennd5b | 20 | 0.396 | 0.469 | −0.243 | 2.55E−05 | 8.06E−03 |
| Ndel1 | 20 | 0.116 | 0.178 | −0.469 | 2.59E−05 | 8.15E−03 |
| Kras | 20 | 0.142 | 0.207 | −0.440 | 2.60E−05 | 8.17E−03 |
| Smim7 | 20 | 0.241 | 0.326 | −0.324 | 2.65E−05 | 8.31E−03 |
| 1810026J23Rik | 20 | 0.094 | 0.153 | −0.443 | 2.67E−05 | 8.34E−03 |
| Atp9a | 20 | 0.128 | 0.19 | −0.423 | 2.71E−05 | 8.46E−03 |
| Yipf2 | 20 | 0.214 | 0.308 | −0.334 | 2.73E−05 | 8.53E−03 |
| Ube2j2 | 20 | 0.272 | 0.366 | −0.335 | 2.77E−05 | 8.63E−03 |
| Trnau1ap | 20 | 0.108 | 0.161 | −0.419 | 2.87E−05 | 8.93E−03 |
| Abhd16a | 20 | 0.135 | 0.206 | −0.394 | 2.90E−05 | 8.99E−03 |
| Bmyc | 20 | 0.13 | 0.184 | −0.383 | 3.01E−05 | 9.31E−03 |
| Denr | 20 | 0.293 | 0.395 | −0.316 | 3.11E−05 | 9.57E−03 |
| Rab2a | 20 | 0.595 | 0.708 | −0.259 | 3.14E−05 | 9.63E−03 |
| Luc7l3 | 20 | 0.14 | 0.181 | −0.363 | 3.23E−05 | 9.88E−03 |
| Ncf4 | 20 | 0.301 | 0.395 | −0.327 | 3.26E−05 | 9.93E−03 |
| Cnbp | 20 | 0.622 | 0.75 | −0.250 | 3.32E−05 | 1.01E−02 |
| Snapc5 | 20 | 0.15 | 0.212 | −0.393 | 3.85E−05 | 1.16E−02 |
| Zfp64 | 20 | 0.126 | 0.193 | −0.454 | 4.04E−05 | 1.20E−02 |
| Sec24c | 20 | 0.127 | 0.191 | −0.408 | 4.05E−05 | 1.21E−02 |
| Zfand2b | 20 | 0.125 | 0.192 | −0.414 | 4.07E−05 | 1.21E−02 |
| Fuca1 | 20 | 0.293 | 0.398 | −0.328 | 4.11E−05 | 1.22E−02 |
| Tmem5 | 20 | 0.105 | 0.162 | −0.413 | 4.23E−05 | 1.25E−02 |
| Rgs10 | 20 | 0.417 | 0.523 | −0.280 | 4.28E−05 | 1.26E−02 |
| Vamp2 | 20 | 0.182 | 0.26 | −0.367 | 4.30E−05 | 1.27E−02 |
| Haus3 | 20 | 0.349 | 0.455 | −0.284 | 4.35E−05 | 1.28E−02 |
| Sept11 | 20 | 0.213 | 0.285 | −0.341 | 4.47E−05 | 1.31E−02 |
| Fam204a | 20 | 0.164 | 0.241 | −0.372 | 4.48E−05 | 1.31E−02 |
| Tap1 | 20 | 0.164 | 0.221 | −0.349 | 4.49E−05 | 1.32E−02 |
| Pex2 | 20 | 0.167 | 0.215 | −0.335 | 4.57E−05 | 1.34E−02 |
| Polr2e | 20 | 0.275 | 0.34 | −0.251 | 4.64E−05 | 1.36E−02 |
| Tmem203 | 20 | 0.147 | 0.219 | −0.362 | 4.65E−05 | 1.36E−02 |
| Grpel1 | 20 | 0.3 | 0.42 | −0.326 | 4.67E−05 | 1.36E−02 |
| Vps37a | 20 | 0.185 | 0.267 | −0.336 | 4.70E−05 | 1.37E−02 |
| Cd164 | 20 | 0.27 | 0.349 | −0.328 | 4.76E−05 | 1.39E−02 |
| Gtf2a2 | 20 | 0.247 | 0.342 | −0.357 | 4.91E−05 | 1.42E−02 |
| Cbx4 | 20 | 0.163 | 0.232 | −0.356 | 4.97E−05 | 1.44E−02 |
| Apitd1 | 20 | 0.156 | 0.202 | −0.370 | 5.12E−05 | 1.47E−02 |
| Rhobtb1 | 20 | 0.195 | 0.272 | −0.377 | 5.16E−05 | 1.48E−02 |
| Strap | 20 | 0.251 | 0.341 | −0.364 | 5.23E−05 | 1.50E−02 |
| Nek7 | 20 | 0.39 | 0.514 | −0.308 | 5.40E−05 | 1.54E−02 |
| Egln2 | 20 | 0.204 | 0.288 | −0.323 | 5.50E−05 | 1.57E−02 |
| Atp6v0d1 | 20 | 0.325 | 0.385 | −0.232 | 5.66E−05 | 1.61E−02 |
| Htatsf1 | 20 | 0.104 | 0.167 | −0.451 | 5.68E−05 | 1.62E−02 |
| Csde1 | 20 | 0.311 | 0.399 | −0.301 | 5.73E−05 | 1.63E−02 |
| Pdcd4 | 20 | 0.301 | 0.391 | −0.267 | 5.87E−05 | 1.66E−02 |
| Cd82 | 20 | 0.192 | 0.234 | −0.256 | 5.97E−05 | 1.69E−02 |
| Man1a2 | 20 | 0.156 | 0.224 | −0.386 | 6.04E−05 | 1.70E−02 |
| Pafah1b1 | 20 | 0.292 | 0.374 | −0.313 | 6.33E−05 | 1.77E−02 |
| Slc48a1 | 20 | 0.254 | 0.346 | −0.314 | 6.68E−05 | 1.85E−02 |
| Kmt2e | 20 | 0.362 | 0.469 | −0.310 | 6.82E−05 | 1.89E−02 |
| Irf2 | 20 | 0.253 | 0.34 | −0.329 | 7.14E−05 | 1.96E−02 |
| Atp6v0b | 20 | 0.48 | 0.589 | −0.242 | 7.17E−05 | 1.96E−02 |
| Yipf4 | 20 | 0.287 | 0.373 | −0.292 | 7.27E−05 | 1.99E−02 |
| Atg3 | 20 | 0.271 | 0.37 | −0.332 | 7.36E−05 | 2.01E−02 |
| Arl8b | 20 | 0.148 | 0.211 | −0.400 | 7.57E−05 | 2.06E−02 |
| Prr5 | 20 | 0.442 | 0.567 | −0.303 | 7.64E−05 | 2.08E−02 |
| Vapb | 20 | 0.167 | 0.245 | −0.396 | 7.81E−05 | 2.12E−02 |
| Eif4enif1 | 20 | 0.13 | 0.183 | −0.382 | 7.98E−05 | 2.16E−02 |
| Ctnnbl1 | 20 | 0.142 | 0.207 | −0.435 | 8.02E−05 | 2.17E−02 |
| Bola3 | 20 | 0.24 | 0.339 | −0.343 | 8.07E−05 | 2.18E−02 |
| Ddx3x | 20 | 0.237 | 0.325 | −0.318 | 8.09E−05 | 2.18E−02 |
| Insig1 | 20 | 0.239 | 0.322 | −0.339 | 8.14E−05 | 2.19E−02 |
| Map3k1 | 20 | 0.124 | 0.166 | −0.341 | 8.16E−05 | 2.20E−02 |
| H2-Q7 | 20 | 0.095 | 0.153 | −0.452 | 8.22E−05 | 2.21E−02 |
| Uvrag | 20 | 0.127 | 0.193 | −0.435 | 8.30E−05 | 2.22E−02 |
| Abcb1b | 20 | 0.096 | 0.154 | −0.442 | 8.55E−05 | 2.28E−02 |
| Phf3 | 20 | 0.176 | 0.218 | −0.261 | 8.77E−05 | 2.33E−02 |
| Fubp1 | 20 | 0.107 | 0.168 | −0.459 | 8.96E−05 | 2.38E−02 |
| Eps15 | 20 | 0.111 | 0.166 | −0.412 | 9.03E−05 | 2.39E−02 |
| 4833439L19Rik | 20 | 0.116 | 0.153 | −0.324 | 9.16E−05 | 2.42E−02 |
| Gpr180 | 20 | 0.153 | 0.202 | −0.308 | 9.17E−05 | 2.42E−02 |
| Cops7a | 20 | 0.178 | 0.261 | −0.380 | 9.26E−05 | 2.44E−02 |
| Lrrc59 | 20 | 0.494 | 0.645 | −0.306 | 9.48E−05 | 2.49E−02 |
| Nus1 | 20 | 0.207 | 0.264 | −0.326 | 9.65E−05 | 2.52E−02 |
| Chuk | 20 | 0.119 | 0.153 | −0.352 | 9.71E−05 | 2.54E−02 |
| Maf1 | 20 | 0.234 | 0.292 | −0.270 | 9.92E−05 | 2.58E−02 |
| Cnot7 | 20 | 0.185 | 0.249 | −0.335 | 1.01E−04 | 2.63E−02 |
| Smim10l1 | 20 | 0.116 | 0.175 | −0.408 | 1.02E−04 | 2.66E−02 |
| Babam1 | 20 | 0.302 | 0.392 | −0.301 | 1.03E−04 | 2.67E−02 |
| Arfgap1 | 20 | 0.126 | 0.181 | −0.404 | 1.14E−04 | 2.92E−02 |
| Eif4g3 | 20 | 0.144 | 0.176 | −0.277 | 1.16E−04 | 2.96E−02 |
| DynIl2 | 20 | 0.193 | 0.272 | −0.357 | 1.19E−04 | 3.04E−02 |
| Coq2 | 20 | 0.094 | 0.151 | −0.449 | 1.22E−04 | 3.09E−02 |
| Pla2g12a | 20 | 0.233 | 0.328 | −0.338 | 1.24E−04 | 3.14E−02 |
| Atg13 | 20 | 0.11 | 0.159 | −0.366 | 1.26E−04 | 3.19E−02 |
| Abhd17b | 20 | 0.128 | 0.192 | −0.439 | 1.26E−04 | 3.19E−02 |
| Raf1 | 20 | 0.234 | 0.324 | −0.364 | 1.29E−04 | 3.25E−02 |
| Ppp1r14b | 20 | 0.175 | 0.253 | −0.391 | 1.30E−04 | 3.27E−02 |
| Chtop | 20 | 0.236 | 0.301 | −0.292 | 1.33E−04 | 3.32E−02 |
| Ccdc127 | 20 | 0.164 | 0.207 | −0.273 | 1.39E−04 | 3.45E−02 |
| Papola | 20 | 0.368 | 0.453 | −0.276 | 1.40E−04 | 3.46E−02 |
| St3gal6 | 20 | 0.168 | 0.223 | −0.332 | 1.42E−04 | 3.50E−02 |
| Bloc1s2 | 20 | 0.142 | 0.202 | −0.417 | 1.45E−04 | 3.55E−02 |
| Ndnl2 | 20 | 0.125 | 0.175 | −0.381 | 1.46E−04 | 3.58E−02 |
| Ccnt2 | 20 | 0.174 | 0.227 | −0.331 | 1.46E−04 | 3.58E−02 |
| Map2k3 | 20 | 0.134 | 0.181 | −0.363 | 1.49E−04 | 3.64E−02 |
| Morf4l1 | 20 | 0.559 | 0.68 | −0.265 | 1.55E−04 | 3.76E−02 |
| Dcaf11 | 20 | 0.185 | 0.255 | −0.353 | 1.69E−04 | 4.05E−02 |
| Mrfap1 | 20 | 0.598 | 0.725 | −0.260 | 1.69E−04 | 4.06E−02 |
| Klhdc2 | 20 | 0.442 | 0.563 | −0.295 | 1.71E−04 | 4.09E−02 |
| Slc25a51 | 20 | 0.143 | 0.196 | −0.368 | 1.72E−04 | 4.11E−02 |
| Papd4 | 20 | 0.149 | 0.208 | −0.336 | 1.72E−04 | 4.11E−02 |
| Brap | 20 | 0.121 | 0.168 | −0.372 | 1.74E−04 | 4.15E−02 |
| Shc1 | 20 | 0.108 | 0.158 | −0.425 | 1.76E−04 | 4.18E−02 |
| Mcfd2 | 20 | 0.126 | 0.187 | −0.385 | 1.77E−04 | 4.22E−02 |
| Csnk1g3 | 20 | 0.276 | 0.336 | −0.228 | 1.80E−04 | 4.26E−02 |
| Dpm1 | 20 | 0.153 | 0.196 | −0.276 | 1.88E−04 | 4.44E−02 |
| Srsf5 | 20 | 0.493 | 0.609 | −0.277 | 1.90E−04 | 4.47E−02 |
| Gps2 | 20 | 0.146 | 0.215 | −0.423 | 1.91E−04 | 4.48E−02 |
| Rnf167 | 20 | 0.154 | 0.211 | −0.344 | 1.91E−04 | 4.49E−02 |
| 1700017B05Rik | 20 | 0.273 | 0.353 | −0.296 | 1.91E−04 | 4.50E−02 |
| Letm1 | 20 | 0.182 | 0.247 | −0.355 | 1.93E−04 | 4.52E−02 |
| Rnf187 | 20 | 0.378 | 0.468 | −0.260 | 1.94E−04 | 4.54E−02 |
| Rnf114 | 20 | 0.143 | 0.18 | −0.283 | 1.95E−04 | 4.56E−02 |
| Eif3a | 20 | 0.314 | 0.424 | −0.332 | 1.95E−04 | 4.56E−02 |
| Rusc1 | 20 | 0.244 | 0.331 | −0.315 | 1.96E−04 | 4.58E−02 |
| Armc1 | 20 | 0.135 | 0.186 | −0.366 | 2.04E−04 | 4.75E−02 |
| Gpr65 | 20 | 0.151 | 0.213 | −0.348 | 2.07E−04 | 4.81E−02 |
| Ubxn4 | 20 | 0.547 | 0.666 | −0.275 | 2.12E−04 | 4.91E−02 |
| Zfp622 | 20 | 0.126 | 0.17 | −0.349 | 2.16E−04 | 4.98E−02 |
| Rnf4 | 20 | 0.206 | 0.26 | −0.314 | 2.16E−04 | 4.99E−02 |
| Ighg1 | 21 | 0.256 | 0.029 | 1.795 | 5.93E−33 | 3.67E−29 |
| Mcpt2 | 21 | 0.202 | 0 | 5.176 | 1.61E−26 | 6.87E−23 |
| Nr4a2 | 21 | 0.546 | 0.834 | −0.933 | 1.44E−22 | 4.38E−19 |
| Ppia | 21 | 1 | 0.997 | 0.329 | 1.64E−18 | 3.62E−15 |
| Slc25a20 | 21 | 0.511 | 0.784 | −0.865 | 6.90E−18 | 1.44E−14 |
| Nfkbia | 21 | 0.763 | 0.443 | 0.993 | 5.20E−17 | 9.89E−14 |
| Hspa1a | 21 | 0.385 | 0.135 | 1.750 | 6.60E−17 | 1.23E−13 |
| Mcpt1 | 21 | 0.205 | 0.021 | 4.061 | 1.16E−16 | 2.11E−13 |
| Tm2d2 | 21 | 0.621 | 0.815 | −0.625 | 2.38E−16 | 4.24E−13 |
| Jun | 21 | 0.609 | 0.443 | 0.934 | 1.06E−15 | 1.79E−12 |
| Iglc1 | 21 | 0.464 | 0.768 | −0.583 | 1.17E−15 | 1.95E−12 |
| Zfand5 | 21 | 0.498 | 0.739 | −0.854 | 9.22E−15 | 1.42E−11 |
| Btg2 | 21 | 0.962 | 0.974 | −0.460 | 1.10E−14 | 1.68E−11 |
| Irf7 | 21 | 0.196 | 0.456 | −1.092 | 2.13E−13 | 2.81E−10 |
| Ckb | 21 | 0.934 | 0.905 | 0.434 | 4.26E−13 | 5.42E−10 |
| Ifi30 | 21 | 0.842 | 0.697 | 0.508 | 4.52E−13 | 5.72E−10 |
| Ly6e | 21 | 0.435 | 0.662 | −0.839 | 5.27E−13 | 6.60E−10 |
| Ifi27l2a | 21 | 0.353 | 0.609 | −1.014 | 9.74E−13 | 1.20E−09 |
| Gapdh | 21 | 0.987 | 0.963 | 0.249 | 3.00E−12 | 3.48E−09 |
| Tnfaip2 | 21 | 0.177 | 0.43 | −1.252 | 3.03E−12 | 3.50E−09 |
| Tuba1b | 21 | 0.741 | 0.573 | 0.851 | 2.00E−11 | 2.10E−08 |
| Igkv1-135 | 21 | 0.123 | 0.396 | −1.236 | 6.93E−11 | 6.87E−08 |
| Rgs2 | 21 | 0.741 | 0.879 | −0.603 | 6.94E−11 | 6.87E−08 |
| Neat1 | 21 | 0.735 | 0.594 | 0.579 | 2.12E−10 | 1.95E−07 |
| Atp5b | 21 | 0.95 | 0.923 | 0.322 | 3.56E−10 | 3.15E−07 |
| Il1r2 | 21 | 0.174 | 0.435 | −0.959 | 3.81E−10 | 3.34E−07 |
| Hfe | 21 | 0.461 | 0.261 | 0.857 | 5.83E−10 | 5.00E−07 |
| Calr | 21 | 0.937 | 0.847 | 0.254 | 9.89E−10 | 8.22E−07 |
| Slc25a5 | 21 | 0.978 | 0.96 | 0.323 | 1.47E−09 | 1.17E−06 |
| Tmem50b | 21 | 0.388 | 0.607 | −0.594 | 1.86E−09 | 1.47E−06 |
| B930036N10Rik | 21 | 0.167 | 0.026 | 1.176 | 2.12E−09 | 1.65E−06 |
| Coro1a | 21 | 0.987 | 0.976 | 0.200 | 2.53E−09 | 1.94E−06 |
| Lpar6 | 21 | 0.315 | 0.517 | −0.790 | 3.20E−09 | 2.38E−06 |
| Vps37b | 21 | 0.164 | 0.393 | −1.167 | 3.62E−09 | 2.68E−06 |
| Man2b1 | 21 | 0.82 | 0.744 | 0.355 | 3.71E−09 | 2.72E−06 |
| Rheb | 21 | 0.852 | 0.918 | −0.319 | 4.00E−09 | 2.93E−06 |
| Tifab | 21 | 0.7 | 0.813 | −0.378 | 5.43E−09 | 3.89E−06 |
| Errfi1 | 21 | 0.246 | 0.454 | −0.863 | 7.19E−09 | 5.06E−06 |
| Ctla2b | 21 | 0.473 | 0.639 | −0.685 | 8.48E−09 | 5.90E−06 |
| Rpn2 | 21 | 0.653 | 0.546 | 0.477 | 8.74E−09 | 6.05E−06 |
| Zfp36 | 21 | 0.918 | 0.916 | 0.470 | 1.29E−08 | 8.73E−06 |
| Junb | 21 | 0.978 | 0.984 | −0.242 | 2.25E−08 | 1.46E−05 |
| Ildr1 | 21 | 0.11 | 0.311 | −1.204 | 3.02E−08 | 1.91E−05 |
| Hspa1b | 21 | 0.17 | 0.053 | 1.234 | 7.86E−08 | 4.55E−05 |
| Bcl2a1d | 21 | 0.886 | 0.852 | 0.372 | 1.59E−07 | 8.79E−05 |
| Dck | 21 | 0.237 | 0.443 | −0.663 | 1.81E−07 | 9.86E−05 |
| Hsp90aa1 | 21 | 0.773 | 0.681 | 0.462 | 2.22E−07 | 1.19E−04 |
| Nr4a1 | 21 | 0.811 | 0.931 | −0.351 | 2.63E−07 | 1.39E−04 |
| Apol10b | 21 | 0.066 | 0.214 | −0.935 | 3.25E−07 | 1.68E−04 |
| Igkc | 21 | 1 | 1 | 0.404 | 3.60E−07 | 1.84E−04 |
| Fndc5 | 21 | 0.322 | 0.515 | −0.592 | 3.83E−07 | 1.96E−04 |
| Ndel1 | 21 | 0.438 | 0.612 | −0.617 | 4.54E−07 | 2.28E−04 |
| Cct8 | 21 | 0.785 | 0.707 | 0.364 | 6.91E−07 | 3.33E−04 |
| Herpud1 | 21 | 0.754 | 0.868 | −0.354 | 7.11E−07 | 3.42E−04 |
| Arl4a | 21 | 0.303 | 0.451 | −0.646 | 7.64E−07 | 3.64E−04 |
| Grasp | 21 | 0.375 | 0.57 | −0.773 | 8.38E−07 | 3.95E−04 |
| Cdc42 | 21 | 0.978 | 0.963 | 0.232 | 9.98E−07 | 4.64E−04 |
| Txnip | 21 | 0.139 | 0.319 | −0.939 | 1.07E−06 | 4.96E−04 |
| Tmem243 | 21 | 0.451 | 0.599 | −0.412 | 1.20E−06 | 5.45E−04 |
| Isg15 | 21 | 0.11 | 0.277 | −0.924 | 1.30E−06 | 5.86E−04 |
| Ptger4 | 21 | 0.3 | 0.158 | 0.743 | 1.50E−06 | 6.65E−04 |
| Rap2a | 21 | 0.429 | 0.245 | 0.789 | 1.51E−06 | 6.68E−04 |
| Runx3 | 21 | 0.42 | 0.565 | −0.524 | 1.60E−06 | 7.07E−04 |
| Itgb7 | 21 | 0.735 | 0.649 | 0.391 | 1.90E−06 | 8.21E−04 |
| Ifngr2 | 21 | 0.536 | 0.683 | −0.382 | 2.01E−06 | 8.59E−04 |
| Atf4 | 21 | 0.59 | 0.802 | −0.561 | 2.12E−06 | 9.02E−04 |
| Arl4c | 21 | 0.183 | 0.348 | −0.993 | 2.50E−06 | 1.04E−03 |
| Ddost | 21 | 0.738 | 0.673 | 0.309 | 2.98E−06 | 1.21E−03 |
| Eef1d | 21 | 0.886 | 0.852 | 0.198 | 3.02E−06 | 1.22E−03 |
| Ifi44l | 21 | 0.17 | 0.306 | −0.572 | 3.44E−06 | 1.38E−03 |
| Pcyt2 | 21 | 0.344 | 0.462 | −0.494 | 3.49E−06 | 1.39E−03 |
| Lztfl1 | 21 | 0.281 | 0.195 | 0.710 | 3.72E−06 | 1.47E−03 |
| Lbh | 21 | 0.517 | 0.628 | −0.370 | 4.19E−06 | 1.63E−03 |
| Ap1s3 | 21 | 0.691 | 0.773 | −0.306 | 4.33E−06 | 1.69E−03 |
| Ifitm3 | 21 | 0.905 | 0.963 | −0.236 | 5.02E−06 | 1.93E−03 |
| Iglc3 | 21 | 0.069 | 0.224 | −0.825 | 5.42E−06 | 2.06E−03 |
| Ifrd1 | 21 | 0.483 | 0.678 | −0.603 | 6.82E−06 | 2.54E−03 |
| Hnrnpa2b1 | 21 | 0.931 | 0.91 | 0.297 | 7.39E−06 | 2.72E−03 |
| Ppp1r2 | 21 | 0.571 | 0.734 | −0.428 | 7.98E−06 | 2.91E−03 |
| Pkm | 21 | 0.839 | 0.826 | 0.302 | 8.35E−06 | 3.03E−03 |
| Tagln2 | 21 | 0.95 | 0.918 | 0.288 | 9.33E−06 | 3.34E−03 |
| Hnrnpa3 | 21 | 0.905 | 0.868 | 0.301 | 1.00E−05 | 3.57E−03 |
| Sgk1 | 21 | 0.268 | 0.438 | −0.750 | 1.01E−05 | 3.57E−03 |
| Ldha | 21 | 0.962 | 0.931 | 0.319 | 1.14E−05 | 3.98E−03 |
| Irf8 | 21 | 0.924 | 0.955 | −0.160 | 1.19E−05 | 4.15E−03 |
| Kdm6b | 21 | 0.691 | 0.58 | 0.457 | 1.21E−05 | 4.21E−03 |
| Adgre5 | 21 | 0.568 | 0.475 | 0.397 | 1.29E−05 | 4.46E−03 |
| Ubb | 21 | 1 | 0.979 | 0.253 | 1.46E−05 | 4.98E−03 |
| Eno1 | 21 | 0.751 | 0.583 | 0.517 | 1.77E−05 | 5.88E−03 |
| S100a13 | 21 | 0.722 | 0.588 | 0.304 | 1.82E−05 | 6.02E−03 |
| A530040E14Rik | 21 | 0.095 | 0.235 | −0.947 | 1.89E−05 | 6.21E−03 |
| Spg21 | 21 | 0.767 | 0.86 | −0.312 | 1.94E−05 | 6.35E−03 |
| Gna13 | 21 | 0.584 | 0.704 | −0.487 | 2.08E−05 | 6.78E−03 |
| Sla | 21 | 0.773 | 0.654 | 0.364 | 2.12E−05 | 6.87E−03 |
| Gns | 21 | 0.363 | 0.515 | −0.504 | 2.16E−05 | 6.97E−03 |
| Vmp1 | 21 | 0.618 | 0.443 | 0.491 | 2.18E−05 | 7.04E−03 |
| Amica1 | 21 | 0.634 | 0.789 | −0.272 | 2.40E−05 | 7.64E−03 |
| Snrpd1 | 21 | 0.694 | 0.652 | 0.267 | 2.47E−05 | 7.82E−03 |
| Adrb2 | 21 | 0.36 | 0.206 | 0.769 | 2.62E−05 | 8.21E−03 |
| Pdia6 | 21 | 0.767 | 0.644 | 0.410 | 2.75E−05 | 8.56E−03 |
| Eif4a1 | 21 | 0.978 | 0.966 | 0.271 | 3.00E−05 | 9.28E−03 |
| Ifi44 | 21 | 0.054 | 0.161 | −1.018 | 3.09E−05 | 9.50E−03 |
| Ppib | 21 | 0.946 | 0.931 | 0.171 | 3.61E−05 | 1.09E−02 |
| Arih2 | 21 | 0.372 | 0.562 | −0.629 | 3.75E−05 | 1.13E−02 |
| Fam162a | 21 | 0.486 | 0.351 | 0.345 | 3.96E−05 | 1.18E−02 |
| Srsf5 | 21 | 0.845 | 0.873 | −0.330 | 4.04E−05 | 1.20E−02 |
| 2200002D01Rik | 21 | 0.142 | 0.269 | −0.786 | 4.76E−05 | 1.39E−02 |
| Tkt | 21 | 0.748 | 0.673 | 0.338 | 4.92E−05 | 1.42E−02 |
| Pa2g4 | 21 | 0.621 | 0.501 | 0.389 | 5.14E−05 | 1.48E−02 |
| Ezr | 21 | 0.521 | 0.391 | 0.319 | 5.23E−05 | 1.50E−02 |
| Fxyd5 | 21 | 0.754 | 0.633 | 0.274 | 5.42E−05 | 1.55E−02 |
| Il1b | 21 | 0.476 | 0.594 | −0.609 | 5.42E−05 | 1.55E−02 |
| Prkar2a | 21 | 0.546 | 0.699 | −0.435 | 5.51E−05 | 1.57E−02 |
| Fbl | 21 | 0.836 | 0.786 | 0.303 | 5.53E−05 | 1.58E−02 |
| Stt3b | 21 | 0.473 | 0.559 | −0.435 | 5.57E−05 | 1.59E−02 |
| Actb | 21 | 1 | 1 | 0.363 | 5.76E−05 | 1.63E−02 |
| Dusp1 | 21 | 0.861 | 0.768 | 0.517 | 6.02E−05 | 1.70E−02 |
| Fam174a | 21 | 0.565 | 0.71 | −0.382 | 6.07E−05 | 1.71E−02 |
| Tiparp | 21 | 0.107 | 0.245 | −0.919 | 6.14E−05 | 1.72E−02 |
| Zfp706 | 21 | 0.877 | 0.881 | −0.202 | 6.26E−05 | 1.75E−02 |
| Baz1a | 21 | 0.555 | 0.501 | 0.343 | 6.50E−05 | 1.81E−02 |
| Hnrnpu | 21 | 0.688 | 0.588 | 0.377 | 6.67E−05 | 1.85E−02 |
| Eif3l | 21 | 0.666 | 0.578 | 0.303 | 6.88E−05 | 1.90E−02 |
| Got2 | 21 | 0.59 | 0.472 | 0.430 | 7.08E−05 | 1.95E−02 |
| Hlx | 21 | 0.136 | 0.285 | −0.749 | 7.12E−05 | 1.96E−02 |
| Magohb | 21 | 0.199 | 0.348 | −0.617 | 7.14E−05 | 1.96E−02 |
| Dgat1 | 21 | 0.088 | 0.214 | −1.018 | 7.42E−05 | 2.03E−02 |
| Cdv3 | 21 | 0.555 | 0.654 | −0.426 | 7.53E−05 | 2.06E−02 |
| Litaf | 21 | 0.694 | 0.799 | −0.273 | 7.86E−05 | 2.13E−02 |
| Igkv12-44 | 21 | 0.174 | 0.077 | 0.985 | 8.44E−05 | 2.26E−02 |
| Napsa | 21 | 0.767 | 0.665 | 0.374 | 8.62E−05 | 2.30E−02 |
| Evi2a | 21 | 0.612 | 0.755 | −0.246 | 9.30E−05 | 2.45E−02 |
| Dusp2 | 21 | 0.407 | 0.264 | 0.608 | 9.44E−05 | 2.48E−02 |
| AC125149.3 | 21 | 0.088 | 0.219 | −0.978 | 9.63E−05 | 2.52E−02 |
| Orai1 | 21 | 0.596 | 0.446 | 0.338 | 1.10E−04 | 2.83E−02 |
| Agpat4 | 21 | 0.511 | 0.673 | −0.476 | 1.10E−04 | 2.84E−02 |
| Mt1 | 21 | 0.486 | 0.319 | 0.475 | 1.11E−04 | 2.84E−02 |
| Ube2l6 | 21 | 0.211 | 0.332 | −0.593 | 1.13E−04 | 2.88E−02 |
| Fndc3a | 21 | 0.098 | 0.232 | −0.888 | 1.14E−04 | 2.91E−02 |
| Gm | 21 | 0.839 | 0.776 | 0.296 | 1.28E−04 | 3.22E−02 |
| Fh1 | 21 | 0.552 | 0.417 | 0.471 | 1.35E−04 | 3.37E−02 |
| Commd8 | 21 | 0.804 | 0.852 | −0.222 | 1.38E−04 | 3.42E−02 |
| Snrnp70 | 21 | 0.517 | 0.406 | 0.365 | 1.42E−04 | 3.51E−02 |
| Dapk2 | 21 | 0.435 | 0.559 | −0.381 | 1.43E−04 | 3.53E−02 |
| Brox | 21 | 0.132 | 0.259 | −0.540 | 1.49E−04 | 3.64E−02 |
| Mnda | 21 | 0.65 | 0.541 | 0.528 | 1.54E−04 | 3.74E−02 |
| Btg1 | 21 | 0.965 | 0.987 | −0.253 | 1.56E−04 | 3.77E−02 |
| Mcm7 | 21 | 0.218 | 0.098 | 0.815 | 1.63E−04 | 3.93E−02 |
| Map1lc3a | 21 | 0.183 | 0.33 | −0.666 | 1.69E−04 | 4.06E−02 |
| Ubald2 | 21 | 0.606 | 0.739 | −0.445 | 1.75E−04 | 4.17E−02 |
| Ptp4a1 | 21 | 0.539 | 0.665 | −0.401 | 1.77E−04 | 4.21E−02 |
| Prr13 | 21 | 0.817 | 0.881 | −0.200 | 1.80E−04 | 4.26E−02 |
| Atxn7l3b | 21 | 0.514 | 0.649 | −0.356 | 1.84E−04 | 4.35E−02 |
| Nlrp3 | 21 | 0.107 | 0.219 | −0.852 | 1.84E−04 | 4.35E−02 |
| Nabp1 | 21 | 0.249 | 0.417 | −0.602 | 1.87E−04 | 4.41E−02 |
| Gngt2 | 21 | 0.338 | 0.478 | −0.369 | 1.93E−04 | 4.52E−02 |
| Chic2 | 21 | 0.647 | 0.768 | −0.314 | 1.94E−04 | 4.55E−02 |
| Tspan13 | 21 | 0.808 | 0.889 | −0.216 | 2.01E−04 | 4.68E−02 |
| Al467606 | 21 | 0.189 | 0.084 | 0.798 | 2.03E−04 | 4.71E−02 |
| Actg1 | 22 | 0.977 | 0.98 | 0.479 | 3.28E−21 | 9.09E−18 |
| Vim | 22 | 0.663 | 0.42 | 0.827 | 1.03E−20 | 2.79E−17 |
| Ccl7 | 22 | 0.286 | 0.037 | 3.742 | 3.69E−19 | 8.76E−16 |
| Ighg1 | 22 | 0.166 | 0.007 | 3.332 | 4.61E−19 | 1.08E−15 |
| AW112010 | 22 | 0.288 | 0.573 | −1.156 | 6.67E−19 | 1.52E−15 |
| Tspan13 | 22 | 0.505 | 0.732 | −0.833 | 4.47E−18 | 9.47E−15 |
| HP | 22 | 0.301 | 0.075 | 1.775 | 2.69E−17 | 5.24E−14 |
| Ccl2 | 22 | 0.416 | 0.139 | 1.980 | 9.74E−16 | 1.65E−12 |
| Fth1 | 22 | 0.995 | 1 | −0.457 | 3.57E−15 | 5.74E−12 |
| Ccl9 | 22 | 0.635 | 0.41 | 0.687 | 1.56E−14 | 2.34E−11 |
| Apol7c | 22 | 0.089 | 0.247 | −1.747 | 2.61E−14 | 3.85E−11 |
| Aif1 | 22 | 0.663 | 0.858 | −0.513 | 3.02E−14 | 4.38E−11 |
| Hspa8 | 22 | 0.936 | 0.878 | 0.333 | 4.03E−14 | 5.76E−11 |
| Dnase1l3 | 22 | 0.171 | 0.366 | −1.262 | 1.12E−13 | 1.50E−10 |
| Cebpb | 22 | 0.977 | 0.905 | 0.489 | 4.18E−13 | 5.33E−10 |
| Dusp1 | 22 | 0.939 | 0.898 | 0.547 | 5.81E−13 | 7.26E−10 |
| Tm2d2 | 22 | 0.304 | 0.441 | −0.730 | 1.09E−12 | 1.34E−09 |
| Irf7 | 22 | 0.24 | 0.471 | −1.077 | 1.15E−12 | 1.40E−09 |
| Mgst1 | 22 | 0.311 | 0.122 | 1.436 | 2.83E−12 | 3.31E−09 |
| Isg15 | 22 | 0.26 | 0.485 | −1.002 | 3.09E−12 | 3.57E−09 |
| Cxcl2 | 22 | 0.781 | 0.549 | 0.778 | 4.23E−12 | 4.81E−09 |
| Cd74 | 22 | 0.857 | 0.966 | −0.387 | 7.80E−12 | 8.58E−09 |
| Ftl1 | 22 | 1 | 1 | −0.297 | 2.50E−11 | 2.61E−08 |
| Irf8 | 22 | 0.393 | 0.583 | −0.747 | 3.02E−11 | 3.11E−08 |
| Chil3 | 22 | 0.153 | 0.02 | 2.589 | 3.95E−11 | 4.03E−08 |
| Lpar6 | 22 | 0.23 | 0.444 | −0.964 | 4.51E−11 | 4.56E−08 |
| Cxcl16 | 22 | 0.454 | 0.644 | −0.679 | 7.38E−11 | 7.28E−08 |
| Asb2 | 22 | 0.112 | 0.302 | −1.341 | 7.54E−11 | 7.41E−08 |
| Napsa | 22 | 0.385 | 0.254 | 0.886 | 7.66E−11 | 7.50E−08 |
| Ccl6 | 22 | 0.806 | 0.631 | 0.287 | 1.11E−10 | 1.07E−07 |
| Tgfb1 | 22 | 0.712 | 0.841 | −0.483 | 1.41E−10 | 1.34E−07 |
| Fn1 | 22 | 0.255 | 0.102 | 1.534 | 3.10E−10 | 2.79E−07 |
| H2-DMb1 | 22 | 0.607 | 0.79 | −0.499 | 3.44E−10 | 3.06E−07 |
| S100a4 | 22 | 0.411 | 0.214 | 0.571 | 3.58E−10 | 3.17E−07 |
| Aldh2 | 22 | 0.464 | 0.336 | 0.668 | 3.60E−10 | 3.18E−07 |
| Arid5a | 22 | 0.158 | 0.356 | −1.089 | 3.65E−10 | 3.21E−07 |
| Malat1 | 22 | 1 | 1 | −0.442 | 5.65E−10 | 4.86E−07 |
| Tmsb4x | 22 | 1 | 1 | −0.261 | 6.35E−10 | 5.40E−07 |
| S100a6 | 22 | 0.548 | 0.437 | 0.632 | 9.21E−10 | 7.70E−07 |
| Batf | 22 | 0.105 | 0.305 | −1.283 | 1.29E−09 | 1.04E−06 |
| Adamdec1 | 22 | 0.163 | 0.312 | −1.284 | 1.48E−09 | 1.18E−06 |
| Igkv1-135 | 22 | 0.02 | 0.163 | −1.953 | 1.89E−09 | 1.48E−06 |
| Rnf149 | 22 | 0.589 | 0.783 | −0.517 | 2.35E−09 | 1.82E−06 |
| H2-Aa | 22 | 0.763 | 0.932 | −0.214 | 2.40E−09 | 1.85E−06 |
| Alox5ap | 22 | 0.548 | 0.4 | 0.711 | 2.80E−09 | 2.13E−06 |
| Gbp7 | 22 | 0.097 | 0.278 | −1.220 | 2.81E−09 | 2.13E−06 |
| Vps37b | 22 | 0.148 | 0.315 | −1.322 | 2.94E−09 | 2.22E−06 |
| F13a1 | 22 | 0.214 | 0.068 | 1.352 | 2.98E−09 | 2.24E−06 |
| Emp3 | 22 | 0.518 | 0.346 | 0.588 | 3.67E−09 | 2.70E−06 |
| Iglc1 | 22 | 0.27 | 0.481 | −0.819 | 4.60E−09 | 3.33E−06 |
| Egr2 | 22 | 0.276 | 0.105 | 1.235 | 8.97E−09 | 6.19E−06 |
| Lair1 | 22 | 0.276 | 0.495 | −0.673 | 1.02E−08 | 6.97E−06 |
| Hjurp | 22 | 0.071 | 0.217 | −1.397 | 1.05E−08 | 7.13E−06 |
| Egr1 | 22 | 0.592 | 0.366 | 0.285 | 1.12E−08 | 7.61E−06 |
| Atp6v0e | 22 | 0.64 | 0.763 | −0.402 | 1.74E−08 | 1.15E−05 |
| Irf1 | 22 | 0.105 | 0.278 | −1.311 | 2.32E−08 | 1.50E−05 |
| S100a10 | 22 | 0.413 | 0.258 | 0.723 | 2.42E−08 | 1.56E−05 |
| Tapbp | 22 | 0.36 | 0.532 | −0.598 | 2.57E−08 | 1.65E−05 |
| Neurl3 | 22 | 0.319 | 0.505 | −0.681 | 2.64E−08 | 1.69E−05 |
| Pla2g2d | 22 | 0.059 | 0.186 | −1.637 | 2.71E−08 | 1.73E−05 |
| Acp5 | 22 | 0.298 | 0.444 | −0.863 | 3.31E−08 | 2.07E−05 |
| Fgl2 | 22 | 0.194 | 0.373 | −0.766 | 3.42E−08 | 2.14E−05 |
| C3 | 22 | 0.235 | 0.095 | 1.228 | 3.80E−08 | 2.35E−05 |
| Mxd1 | 22 | 0.133 | 0.292 | −0.960 | 5.12E−08 | 3.07E−05 |
| Naca | 22 | 0.86 | 0.756 | 0.200 | 5.85E−08 | 3.47E−05 |
| Pf4 | 22 | 0.344 | 0.268 | 1.093 | 6.03E−08 | 3.57E−05 |
| H2-DMb2 | 22 | 0.434 | 0.62 | −0.540 | 6.59E−08 | 3.88E−05 |
| Ctsz | 22 | 0.821 | 0.854 | −0.372 | 7.46E−08 | 4.34E−05 |
| Batf3 | 22 | 0.276 | 0.464 | −0.688 | 7.85E−08 | 4.55E−05 |
| Ppp1r15a | 22 | 0.446 | 0.542 | −0.637 | 9.41E−08 | 5.37E−05 |
| Coro1a | 22 | 0.844 | 0.776 | 0.338 | 9.49E−08 | 5.41E−05 |
| Anxa1 | 22 | 0.286 | 0.159 | 1.069 | 1.07E−07 | 6.04E−05 |
| Svbp | 22 | 0.179 | 0.346 | −0.827 | 1.14E−07 | 6.44E−05 |
| Btg1 | 22 | 0.916 | 0.959 | −0.344 | 1.25E−07 | 7.03E−05 |
| Hpgds | 22 | 0.105 | 0.251 | −0.982 | 1.41E−07 | 7.87E−05 |
| Gda | 22 | 0.258 | 0.105 | 1.080 | 1.49E−07 | 8.26E−05 |
| Srgn | 22 | 0.895 | 0.81 | 0.343 | 1.66E−07 | 9.13E−05 |
| Ptgs1 | 22 | 0.23 | 0.373 | −0.825 | 1.67E−07 | 9.14E−05 |
| Anxa2 | 22 | 0.418 | 0.312 | 0.668 | 2.08E−07 | 1.12E−04 |
| Cd37 | 22 | 0.184 | 0.064 | 1.128 | 2.27E−07 | 1.21E−04 |
| Rnasel | 22 | 0.097 | 0.258 | −1.028 | 2.29E−07 | 1.22E−04 |
| Socs3 | 22 | 0.258 | 0.4 | −0.880 | 2.56E−07 | 1.35E−04 |
| Grb2 | 22 | 0.383 | 0.553 | −0.530 | 2.69E−07 | 1.41E−04 |
| AB124611 | 22 | 0.342 | 0.197 | 0.557 | 2.88E−07 | 1.51E−04 |
| H2-Eb1 | 22 | 0.719 | 0.875 | −0.162 | 3.43E−07 | 1.76E−04 |
| Herpud1 | 22 | 0.281 | 0.427 | −0.720 | 3.65E−07 | 1.87E−04 |
| Marcks | 22 | 0.571 | 0.759 | −0.384 | 4.35E−07 | 2.20E−04 |
| Plin2 | 22 | 0.679 | 0.515 | 0.440 | 4.85E−07 | 2.42E−04 |
| Ahnak | 22 | 0.37 | 0.21 | 0.634 | 5.19E−07 | 2.58E−04 |
| Fxyd5 | 22 | 0.651 | 0.559 | 0.399 | 6.21E−07 | 3.03E−04 |
| P2ry14 | 22 | 0.064 | 0.186 | −1.294 | 6.30E−07 | 3.07E−04 |
| Hbegf | 22 | 0.179 | 0.047 | 1.831 | 6.69E−07 | 3.23E−04 |
| Srsf5 | 22 | 0.64 | 0.664 | −0.284 | 6.84E−07 | 3.30E−04 |
| C130050O18Rik | 22 | 0.171 | 0.264 | −0.678 | 8.69E−07 | 4.08 E−04 |
| Ier3 | 22 | 0.523 | 0.339 | 0.543 | 9.39E−07 | 4.39E−04 |
| Slco3a1 | 22 | 0.048 | 0.166 | −1.361 | 1.00E−06 | 4.66E−04 |
| Lgals3bp | 22 | 0.171 | 0.339 | −0.835 | 1.10E−06 | 5.06E−04 |
| Csf1r | 22 | 0.727 | 0.776 | −0.486 | 1.14E−06 | 5.25E−04 |
| S100a11 | 22 | 0.543 | 0.434 | 0.494 | 1.18E−06 | 5.37E−04 |
| Cfp | 22 | 0.556 | 0.38 | 0.344 | 1.23E−06 | 5.60E−04 |
| Klrk1 | 22 | 0.059 | 0.186 | −1.286 | 1.31E−06 | 5.91E−04 |
| Gbp2b | 22 | 0.107 | 0.241 | −0.981 | 1.35E−06 | 6.05E−04 |
| Nr4a1 | 22 | 0.666 | 0.492 | 0.369 | 1.40E−06 | 6.26E−04 |
| Prdm1 | 22 | 0.189 | 0.373 | −0.693 | 1.47E−06 | 6.54E−04 |
| Fam46c | 22 | 0.061 | 0.197 | −1.150 | 1.61E−06 | 7.10E−04 |
| Rcan1 | 22 | 0.168 | 0.044 | 1.957 | 1.71E−06 | 7.50E−04 |
| Hspa1a | 22 | 0.214 | 0.095 | 0.524 | 1.73E−06 | 7.56E−04 |
| Cdk2ap2 | 22 | 0.561 | 0.695 | −0.473 | 1.80E−06 | 7.85E−04 |
| Clec10a | 22 | 0.227 | 0.098 | 1.028 | 1.93E−06 | 8.32E−04 |
| Fam46a | 22 | 0.207 | 0.383 | −0.803 | 1.95E−06 | 8.37E−04 |
| Cdc42se2 | 22 | 0.194 | 0.353 | −0.800 | 1.95E−06 | 8.37E−04 |
| Ifnar1 | 22 | 0.227 | 0.329 | −0.654 | 2.13E−06 | 9.07E−04 |
| Cd44 | 22 | 0.625 | 0.464 | 0.421 | 2.16E−06 | 9.16E−04 |
| Pltp | 22 | 0.375 | 0.227 | 0.817 | 2.21E−06 | 9.35E−04 |
| Npm1 | 22 | 0.781 | 0.668 | 0.231 | 2.33E−06 | 9.84E−04 |
| Map3k8 | 22 | 0.082 | 0.217 | −1.011 | 2.36E−06 | 9.96E−04 |
| Runx3 | 22 | 0.332 | 0.441 | −0.480 | 2.40E−06 | 1.01E−03 |
| Ntpcr | 22 | 0.173 | 0.298 | −0.826 | 2.45E−06 | 1.03E−03 |
| Peli1 | 22 | 0.207 | 0.366 | −0.754 | 2.49E−06 | 1.04E−03 |
| Rbm7 | 22 | 0.357 | 0.536 | −0.576 | 2.52E−06 | 1.05E−03 |
| Psme2 | 22 | 0.523 | 0.624 | −0.403 | 2.56E−06 | 1.06E−03 |
| Hn1 | 22 | 0.485 | 0.614 | −0.424 | 2.76E−06 | 1.14E−03 |
| Msrb1 | 22 | 0.569 | 0.478 | 0.573 | 2.83E−06 | 1.16E−03 |
| Mgat4a | 22 | 0.102 | 0.227 | −0.897 | 3.15E−06 | 1.27E−03 |
| Cd14 | 22 | 0.801 | 0.668 | 0.328 | 3.26E−06 | 1.31E−03 |
| Osm | 22 | 0.385 | 0.234 | 1.039 | 3.30E−06 | 1.32E−03 |
| Rgs2 | 22 | 0.518 | 0.705 | −0.605 | 3.56E−06 | 1.42E−03 |
| Pgd | 22 | 0.288 | 0.156 | 0.747 | 3.87E−06 | 1.52E−03 |
| Zfp263 | 22 | 0.219 | 0.353 | −0.601 | 4.01E−06 | 1.57E−03 |
| Bst2 | 22 | 0.421 | 0.502 | −0.428 | 4.37E−06 | 1.70E−03 |
| Tcirg1 | 22 | 0.253 | 0.153 | 0.290 | 4.44E−06 | 1.72E−03 |
| Lbh | 22 | 0.27 | 0.407 | −0.695 | 5.04E−06 | 1.94E−03 |
| Fam26f | 22 | 0.094 | 0.22 | −1.012 | 5.94E−06 | 2.24E−03 |
| Etv3 | 22 | 0.191 | 0.353 | −0.678 | 6.06E−06 | 2.28E−03 |
| Fosb | 22 | 0.385 | 0.461 | −0.614 | 6.07E−06 | 2.29E−03 |
| B2m | 22 | 0.98 | 0.963 | −0.230 | 6.16E−06 | 2.32E−03 |
| Actb | 22 | 0.992 | 1 | 0.400 | 6.22E−06 | 2.34E−03 |
| Calm1 | 22 | 0.791 | 0.831 | −0.340 | 6.37E−06 | 2.39E−03 |
| Nfkbie | 22 | 0.117 | 0.254 | −0.800 | 6.52E−06 | 2.44E−03 |
| Fyb | 22 | 0.492 | 0.614 | −0.489 | 6.64E−06 | 2.47E−03 |
| Hic1 | 22 | 0.077 | 0.19 | −1.211 | 7.07E−06 | 2.62E−03 |
| Abi3 | 22 | 0.145 | 0.281 | −0.957 | 7.26E−06 | 2.68E−03 |
| Tgfbr1 | 22 | 0.151 | 0.305 | −0.751 | 7.42E−06 | 2.73E−03 |
| Il6st | 22 | 0.092 | 0.22 | −0.914 | 7.63E−06 | 2.80E−03 |
| Ly6c1 | 22 | 0.296 | 0.169 | 0.222 | 7.93E−06 | 2.90E−03 |
| Cd83 | 22 | 0.717 | 0.834 | −0.322 | 7.96E−06 | 2.91E−03 |
| Slc25a5 | 22 | 0.742 | 0.675 | 0.258 | 8.41E−06 | 3.05E−03 |
| Tm6sf1 | 22 | 0.477 | 0.322 | 0.432 | 8.82E−06 | 3.18E−03 |
| Ybx1 | 22 | 0.788 | 0.817 | −0.292 | 9.72E−06 | 3.47E−03 |
| Abr | 22 | 0.117 | 0.247 | −0.901 | 1.06E−05 | 3.76E−03 |
| Gbp4 | 22 | 0.071 | 0.18 | −1.162 | 1.13E−05 | 3.96E−03 |
| Btg2 | 22 | 0.849 | 0.912 | −0.402 | 1.34E−05 | 4.60E−03 |
| AF251705 | 22 | 0.533 | 0.637 | −0.460 | 1.35E−05 | 4.66E−03 |
| Eif3f | 22 | 0.816 | 0.759 | 0.238 | 1.36E−05 | 4.67E−03 |
| Clta | 22 | 0.773 | 0.692 | 0.232 | 1.46E−05 | 4.95E−03 |
| Emp1 | 22 | 0.281 | 0.169 | 0.835 | 1.52E−05 | 5.16E−03 |
| Ifitm6 | 22 | 0.418 | 0.278 | 0.474 | 1.57E−05 | 5.31E−03 |
| Pole4 | 22 | 0.196 | 0.363 | −0.595 | 1.60E−05 | 5.40E−03 |
| Gm2a | 22 | 0.788 | 0.875 | −0.337 | 1.66E−05 | 5.56E−03 |
| Cd81 | 22 | 0.398 | 0.58 | −0.407 | 1.69E−05 | 5.65E−03 |
| Clec2d | 22 | 0.13 | 0.285 | −0.693 | 1.70E−05 | 5.68E−03 |
| H2-K1 | 22 | 0.821 | 0.902 | −0.266 | 1.78E−05 | 5.89E−03 |
| Eif3e | 22 | 0.605 | 0.485 | 0.375 | 1.82E−05 | 6.02E−03 |
| Arhgap17 | 22 | 0.176 | 0.292 | −0.753 | 1.87E−05 | 6.16E−03 |
| S100a13 | 22 | 0.449 | 0.325 | 0.495 | 2.10E−05 | 6.82E−03 |
| Jun | 22 | 0.64 | 0.498 | 0.332 | 2.12E−05 | 6.87E−03 |
| Tnfaip3 | 22 | 0.283 | 0.39 | −0.612 | 2.13E−05 | 6.90E−03 |
| Dnajb6 | 22 | 0.347 | 0.451 | −0.468 | 2.16E−05 | 6.97E−03 |
| Mtpn | 22 | 0.388 | 0.485 | −0.430 | 2.19E−05 | 7.06E−03 |
| Srrm2 | 22 | 0.436 | 0.515 | −0.404 | 2.21E−05 | 7.09E−03 |
| Dab2 | 22 | 0.311 | 0.183 | 0.890 | 2.39E−05 | 7.60E−03 |
| BC005537 | 22 | 0.564 | 0.424 | 0.457 | 2.51E−05 | 7.95E−03 |
| Bcl2l11 | 22 | 0.151 | 0.288 | −0.756 | 2.59E−05 | 8.14E−03 |
| Slpi | 22 | 0.224 | 0.122 | 0.469 | 2.65E−05 | 8.31E−03 |
| Pbxip1 | 22 | 0.209 | 0.292 | −0.553 | 2.66E−05 | 8.33E−03 |
| Ccl3 | 22 | 0.622 | 0.454 | 0.647 | 2.69E−05 | 8.40E−03 |
| Btf3 | 22 | 0.768 | 0.729 | 0.190 | 2.71E−05 | 8.46E−03 |
| Metrnl | 22 | 0.561 | 0.441 | 0.405 | 2.80E−05 | 8.71E−03 |
| Ifi27l2a | 22 | 0.811 | 0.841 | −0.439 | 2.91E−05 | 9.02E−03 |
| Lyz2 | 22 | 0.995 | 0.983 | 0.273 | 2.96E−05 | 9.15E−03 |
| Litaf | 22 | 0.62 | 0.692 | −0.289 | 3.07E−05 | 9.44E−03 |
| Icam1 | 22 | 0.179 | 0.312 | −0.751 | 3.19E−05 | 9.78E−03 |
| Hopx | 22 | 0.25 | 0.149 | 0.893 | 3.34E−05 | 1.02E−02 |
| Cd86 | 22 | 0.388 | 0.376 | −0.227 | 3.37E−05 | 1.02E−02 |
| Cx3cr1 | 22 | 0.327 | 0.407 | −0.597 | 3.43E−05 | 1.04E−02 |
| Cox4i1 | 22 | 0.918 | 0.858 | 0.173 | 3.55E−05 | 1.08E−02 |
| C5ar1 | 22 | 0.393 | 0.241 | 0.701 | 3.68E−05 | 1.11E−02 |
| PISD | 22 | 0.173 | 0.251 | −0.584 | 3.68E−05 | 1.11E−02 |
| Ythdc1 | 22 | 0.151 | 0.285 | −0.753 | 3.68E−05 | 1.11E−02 |
| Rcbtb2 | 22 | 0.219 | 0.376 | −0.737 | 3.90E−05 | 1.17E−02 |
| Il17ra | 22 | 0.237 | 0.125 | 0.712 | 4.07E−05 | 1.21E−02 |
| Ocstamp | 22 | 0.051 | 0.153 | −1.131 | 4.14E−05 | 1.23E−02 |
| Dusp5 | 22 | 0.298 | 0.403 | −0.519 | 4.48E−05 | 1.31E−02 |
| Asph | 22 | 0.194 | 0.302 | −0.601 | 4.64E−05 | 1.36E−02 |
| Pkm | 22 | 0.597 | 0.512 | 0.363 | 4.83E−05 | 1.40E−02 |
| Sdc4 | 22 | 0.362 | 0.254 | 0.962 | 4.87E−05 | 1.41E−02 |
| Csnk1e | 22 | 0.135 | 0.251 | −0.720 | 4.94E−05 | 1.43E−02 |
| Tmem37 | 22 | 0.11 | 0.231 | −0.914 | 4.94E−05 | 1.43E−02 |
| Ndufa1 | 22 | 0.462 | 0.492 | −0.251 | 5.07E−05 | 1.46E−02 |
| P2ry6 | 22 | 0.321 | 0.468 | −0.500 | 5.10E−05 | 1.47E−02 |
| Idh3b | 22 | 0.242 | 0.163 | 0.312 | 5.74E−05 | 1.63E−02 |
| Oser1 | 22 | 0.401 | 0.536 | −0.408 | 5.83E−05 | 1.65E−02 |
| Ptms | 22 | 0.459 | 0.553 | −0.371 | 5.98E−05 | 1.69E−02 |
| Mpeg1 | 22 | 0.778 | 0.871 | −0.225 | 6.14E−05 | 1.72E−02 |
| Ciita | 22 | 0.056 | 0.156 | −1.102 | 6.16E−05 | 1.73E−02 |
| Bri3 | 22 | 0.597 | 0.698 | −0.331 | 6.19E−05 | 1.73E−02 |
| Ighm | 22 | 0.347 | 0.505 | −0.561 | 6.40E−05 | 1.79E−02 |
| Ccl24 | 22 | 0.63 | 0.576 | 0.745 | 6.83E−05 | 1.89E−02 |
| Npl | 22 | 0.066 | 0.153 | −1.103 | 7.04E−05 | 1.94E−02 |
| P2rx4 | 22 | 0.309 | 0.403 | −0.462 | 7.13E−05 | 1.96E−02 |
| Ubl3 | 22 | 0.559 | 0.627 | −0.295 | 7.66E−05 | 2.09E−02 |
| Adam19 | 22 | 0.128 | 0.264 | −0.609 | 8.38E−05 | 2.24E−02 |
| Ndufa4 | 22 | 0.5 | 0.634 | −0.377 | 8.49E−05 | 2.27E−02 |
| Ube2s | 22 | 0.403 | 0.502 | −0.395 | 8.53E−05 | 2.28E−02 |
| Tsc22d3 | 22 | 0.543 | 0.407 | 0.418 | 8.71E−05 | 2.32E−02 |
| Il1b | 22 | 0.867 | 0.929 | −0.351 | 9.72E−05 | 2.54E−02 |
| Sertad1 | 22 | 0.265 | 0.149 | 0.664 | 9.82E−05 | 2.56E−02 |
| Psmb8 | 22 | 0.564 | 0.668 | −0.341 | 1.06E−04 | 2.75E−02 |
| Usf2 | 22 | 0.278 | 0.383 | −0.487 | 1.09E−04 | 2.80E−02 |
| Sgms1 | 22 | 0.179 | 0.081 | 1.009 | 1.11E−04 | 2.85E−02 |
| Gapdh | 22 | 0.679 | 0.624 | 0.201 | 1.16E−04 | 2.96E−02 |
| Arf2 | 22 | 0.173 | 0.071 | 1.044 | 1.20E−04 | 3.04E−02 |
| Tuba1c | 22 | 0.321 | 0.203 | 0.731 | 1.24E−04 | 3.14E−02 |
| Ogfrl1 | 22 | 0.181 | 0.305 | −0.670 | 1.25E−04 | 3.15E−02 |
| Tsc22d1 | 22 | 0.11 | 0.234 | −1.009 | 1.36E−04 | 3.38E−02 |
| Bcl3 | 22 | 0.128 | 0.237 | −0.835 | 1.39E−04 | 3.43E−02 |
| Vasp | 22 | 0.434 | 0.536 | −0.432 | 1.41E−04 | 3.49E−02 |
| Eno1 | 22 | 0.518 | 0.431 | 0.380 | 1.43E−04 | 3.54E−02 |
| Srrm1 | 22 | 0.191 | 0.346 | −0.711 | 1.44E−04 | 3.54E−02 |
| Pmaip1 | 22 | 0.403 | 0.549 | −0.422 | 1.45E−04 | 3.55E−02 |
| Ier2 | 22 | 0.763 | 0.644 | 0.258 | 1.46E−04 | 3.58E−02 |
| Birc3 | 22 | 0.27 | 0.346 | −0.538 | 1.48E−04 | 3.63E−02 |
| Smpdl3a | 22 | 0.51 | 0.441 | 0.442 | 1.49E−04 | 3.63E−02 |
| Csf2rb | 22 | 0.411 | 0.532 | −0.439 | 1.51E−04 | 3.68E−02 |
| Smim3 | 22 | 0.245 | 0.353 | −0.460 | 1.55E−04 | 3.76E−02 |
| Uqcrh | 22 | 0.806 | 0.759 | 0.180 | 1.63E−04 | 3.93E−02 |
| Trem2 | 22 | 0.168 | 0.081 | 0.903 | 1.66E−04 | 3.98E−02 |
| Pxdc1 | 22 | 0.102 | 0.21 | −0.875 | 1.71E−04 | 4.10E−02 |
| Tor3a | 22 | 0.099 | 0.207 | −0.772 | 1.72E−04 | 4.11E−02 |
| Themis2 | 22 | 0.077 | 0.159 | −1.051 | 1.73E−04 | 4.14E−02 |
| Ltb4r1 | 22 | 0.176 | 0.085 | 0.704 | 1.81E−04 | 4.28E−02 |
| Slc15a3 | 22 | 0.383 | 0.454 | −0.576 | 1.84E−04 | 4.35E−02 |
| Nabp1 | 22 | 0.186 | 0.305 | −0.758 | 1.90E−04 | 4.46E−02 |
| Ppfia4 | 22 | 0.306 | 0.451 | −0.532 | 1.91E−04 | 4.49E−02 |
| Taldo1 | 22 | 0.625 | 0.573 | 0.335 | 2.00E−04 | 4.67E−02 |
| Eif4a2 | 22 | 0.418 | 0.295 | 0.493 | 2.15E−04 | 4.96E−02 |
| Tnip3 | 23 | 0 | 0.4 | −6.333 | 8.83E−08 | 5.06E−05 |
| Psap | 23 | 0.21 | 0.667 | −1.655 | 1.11E−04 | 2.85E−02 |
| Dock10 | 24 | 0.926 | 0.489 | 0.417 | 9.58E−05 | 2.51E−02 |
| Mcpt4 | 26 | 0.911 | 0.357 | 1.787 | 5.23E−28 | 2.42E−24 |
| Tph1 | 26 | 0.934 | 0.486 | 1.478 | 2.57E−27 | 1.14E−23 |
| Srsf5 | 26 | 0.63 | 0.829 | −1.082 | 4.98E−21 | 1.37E−17 |
| Cma1 | 26 | 0.744 | 0.243 | 2.537 | 9.01E−19 | 2.03E−15 |
| Fcer1a | 26 | 0.99 | 0.943 | 0.660 | 4.49E−18 | 9.47E−15 |
| Hdc | 26 | 0.851 | 0.5 | 1.578 | 2.26E−17 | 4.45E−14 |
| Mcpt1 | 26 | 0.926 | 0.514 | 0.960 | 4.48E−17 | 8.57E−14 |
| Mcpt2 | 26 | 0.925 | 0.571 | 1.316 | 3.45E−16 | 6.06E−13 |
| Jun | 26 | 0.931 | 0.657 | 0.914 | 1.05E−15 | 1.76E−12 |
| Rac2 | 26 | 0.967 | 0.943 | 0.602 | 3.12E−14 | 4.51E−11 |
| Ftl1 | 26 | 1 | 1 | 0.693 | 1.20E−13 | 1.60E−10 |
| Rhob | 26 | 0.376 | 0.014 | 3.404 | 1.65E−13 | 2.19E−10 |
| Lxn | 26 | 0.462 | 0.071 | 2.252 | 3.85E−13 | 4.92E−10 |
| Rgs13 | 26 | 0.73 | 0.3 | 1.533 | 1.69E−12 | 2.04E−09 |
| Gzmb | 26 | 0.745 | 0.729 | −0.968 | 6.66E−12 | 7.38E−09 |
| Smpx | 26 | 0.629 | 0.229 | 1.654 | 7.19E−12 | 7.96E−09 |
| Sla | 26 | 0.535 | 0.114 | 1.631 | 8.46E−12 | 9.27E−09 |
| Sox4 | 26 | 0.025 | 0.243 | −3.162 | 1.51E−11 | 1.61E−08 |
| Zfp36l2 | 26 | 0.897 | 0.643 | 1.135 | 2.91E−11 | 3.01E−08 |
| Srgn | 26 | 0.999 | 1 | 0.549 | 4.41E−11 | 4.48 E−08 |
| Cpa3 | 26 | 0.999 | 0.986 | 0.828 | 7.69E−11 | 7.52E−08 |
| Ranbp1 | 26 | 0.357 | 0.629 | −0.883 | 9.59E−11 | 9.26E−08 |
| Tgm2 | 26 | 0.011 | 0.186 | −3.066 | 1.04E−10 | 1.00E−07 |
| Ninj1 | 26 | 0.254 | 0.6 | −1.060 | 1.42E−10 | 1.34E−07 |
| Cnbp | 26 | 0.626 | 0.857 | −0.661 | 1.96E−10 | 1.81E−07 |
| Cited2 | 26 | 0.8 | 0.471 | 1.214 | 2.62E−10 | 2.38E−07 |
| Mcpt8 | 26 | 0.171 | 0.457 | −0.992 | 2.92E−10 | 2.64E−07 |
| Btg2 | 26 | 0.23 | 0.571 | −1.300 | 5.60E−10 | 4.82E−07 |
| Hmgn1 | 26 | 0.23 | 0.486 | −1.120 | 9.79E−10 | 8.16E−07 |
| Oaf | 26 | 0.524 | 0.157 | 1.769 | 1.28E−09 | 1.04E−06 |
| Klf2 | 26 | 0.452 | 0.129 | 2.040 | 1.72E−09 | 1.36E−06 |
| Mif | 26 | 0.297 | 0.5 | −0.971 | 1.88E−09 | 1.48E−06 |
| Cst3 | 26 | 0.861 | 0.629 | 0.718 | 2.95E−09 | 2.22E−06 |
| Dnaja1 | 26 | 0.545 | 0.7 | −0.770 | 3.19E−09 | 2.38E−06 |
| Ncl | 26 | 0.46 | 0.671 | −0.777 | 3.37E−09 | 2.51E−06 |
| Il1rl1 | 26 | 0.755 | 0.443 | 1.096 | 4.41E−09 | 3.20E−06 |
| Serpinb1a | 26 | 0.826 | 0.571 | 1.037 | 6.33E−09 | 4.49E−06 |
| Ero1l | 26 | 0.676 | 0.357 | 1.295 | 8.15E−09 | 5.69E−06 |
| Zfp36 | 26 | 0.673 | 0.343 | 1.037 | 1.18E−08 | 7.97E−06 |
| Sub1 | 26 | 0.943 | 0.971 | −0.456 | 3.26E−08 | 2.05E−05 |
| Srsf6 | 26 | 0.347 | 0.6 | −0.812 | 3.47E−08 | 2.16E−05 |
| Srsf2 | 26 | 0.413 | 0.529 | −0.824 | 3.56E−08 | 2.21E−05 |
| Phlda1 | 26 | 0.409 | 0.1 | 2.714 | 5.58E−08 | 3.33E−05 |
| Mctp1 | 26 | 0.419 | 0.129 | 1.488 | 5.82E−08 | 3.46E−05 |
| Crip1 | 26 | 0.883 | 0.743 | 0.753 | 6.64E−08 | 3.90E−05 |
| Spint2 | 26 | 0.088 | 0.314 | −1.410 | 6.72E−08 | 3.93E−05 |
| Ctla2a | 26 | 0.18 | 0 | 4.696 | 6.75E−08 | 3.95E−05 |
| Hic1 | 26 | 0.252 | 0.543 | −1.130 | 7.28E−08 | 4.24E−05 |
| Srrm2 | 26 | 0.369 | 0.614 | −0.891 | 8.77E−08 | 5.03E−05 |
| Cirbp | 26 | 0.334 | 0.571 | −0.866 | 8.94E−08 | 5.12E−05 |
| Tpsb2 | 26 | 0.237 | 0.029 | 4.861 | 9.22E−08 | 5.26E−05 |
| Tmem64 | 26 | 0.346 | 0.057 | 1.990 | 1.20E−07 | 6.78E−05 |
| Npc2 | 26 | 0.789 | 0.6 | 0.654 | 1.21E−07 | 6.84E−05 |
| Klf6 | 26 | 0.581 | 0.329 | 1.376 | 1.64E−07 | 9.03E−05 |
| Hspa1a | 26 | 0.166 | 0 | 5.537 | 1.77E−07 | 9.65E−05 |
| Vim | 26 | 0.626 | 0.343 | 0.759 | 1.88E−07 | 1.02E−04 |
| Eif5a | 26 | 0.721 | 0.786 | −0.446 | 2.15E−07 | 1.15E−04 |
| Furin | 26 | 0.945 | 0.871 | 0.737 | 2.23E−07 | 1.19E−04 |
| Gadd45a | 26 | 0.273 | 0.6 | −0.836 | 2.42E−07 | 1.29E−04 |
| Tgif1 | 26 | 0.046 | 0.243 | −1.699 | 2.49E−07 | 1.32E−04 |
| H2-D1 | 26 | 0.896 | 0.957 | −0.482 | 2.71E−07 | 1.43E−04 |
| Tsc22d3 | 26 | 0.662 | 0.357 | 1.032 | 2.94E−07 | 1.53E−04 |
| Magohb | 26 | 0.165 | 0.429 | −1.075 | 3.01E−07 | 1.57E−04 |
| Igkv1-135 | 26 | 0.033 | 0.2 | −1.693 | 3.48E−07 | 1.78E−04 |
| Gpr132 | 26 | 0.057 | 0.271 | −1.568 | 4.28E−07 | 2.16E−04 |
| Kdelr1 | 26 | 0.317 | 0.071 | 1.679 | 5.15E−07 | 2.57E−04 |
| Il4ra | 26 | 0.147 | 0.286 | −1.265 | 5.76E−07 | 2.83E−04 |
| Ebi3 | 26 | 0.161 | 0 | 4.036 | 5.83E−07 | 2.87E−04 |
| Cyp11a1 | 26 | 0.899 | 0.786 | 0.600 | 7.08E−07 | 3.41E−04 |
| Errfi1 | 26 | 0.365 | 0.529 | −0.821 | 7.10E−07 | 3.42E−04 |
| Plxdc2 | 26 | 0.163 | 0 | 3.918 | 7.20E−07 | 3.45E−04 |
| Slc7a8 | 26 | 0.37 | 0.129 | 1.625 | 9.70E−07 | 4.52E−04 |
| Eif4a1 | 26 | 0.773 | 0.814 | −0.432 | 1.12E−06 | 5.13E−04 |
| Cst7 | 26 | 0.665 | 0.4 | 0.794 | 1.16E−06 | 5.32E−04 |
| Gpr171 | 26 | 0.608 | 0.343 | 1.297 | 1.45E−06 | 6.45E−04 |
| Rnf128 | 26 | 0.374 | 0.1 | 1.404 | 1.78E−06 | 7.77E−04 |
| H2-K1 | 26 | 0.642 | 0.771 | −0.585 | 1.84E−06 | 7.97E−04 |
| Cish | 26 | 0.397 | 0.1 | 1.408 | 1.92E−06 | 8.25E−04 |
| H3f3b | 26 | 0.996 | 0.986 | −0.452 | 1.92E−06 | 8.25E−04 |
| Fam105a | 26 | 0.73 | 0.557 | 0.743 | 2.06E−06 | 8.80E−04 |
| Malat1 | 26 | 0.999 | 1 | −0.422 | 2.21E−06 | 9.37E−04 |
| Neurl3 | 26 | 0.389 | 0.643 | −0.790 | 2.58E−06 | 1.07E−03 |
| Egr1 | 26 | 0.849 | 0.614 | 0.220 | 2.58E−06 | 1.07E−03 |
| Map1lc3a | 26 | 0.378 | 0.514 | −0.642 | 2.71E−06 | 1.12E−03 |
| Sec11c | 26 | 0.715 | 0.514 | 0.729 | 2.79E−06 | 1.15E−03 |
| Pnrc1 | 26 | 0.562 | 0.771 | −0.684 | 2.83E−06 | 1.16E−03 |
| Lgals1 | 26 | 0.7 | 0.429 | 0.520 | 3.68E−06 | 1.46E−03 |
| Lilrb4a | 26 | 0.598 | 0.3 | 0.964 | 4.14E−06 | 1.62E−03 |
| Pilrb2 | 26 | 0.263 | 0.043 | 1.825 | 4.67E−06 | 1.81E−03 |
| Actg1 | 26 | 0.976 | 0.914 | 0.322 | 5.47E−06 | 2.08E−03 |
| Ddit4 | 26 | 0.369 | 0.129 | 1.501 | 5.56E−06 | 2.11E−03 |
| Cyb5a | 26 | 0.509 | 0.243 | 0.906 | 6.23E−06 | 2.34E−03 |
| Ccl2 | 26 | 0.455 | 0.186 | 1.605 | 7.97E−06 | 2.91E−03 |
| Slc30a2 | 26 | 0.206 | 0.029 | 1.497 | 7.99E−06 | 2.91E−03 |
| Vwa5a | 26 | 0.338 | 0.1 | 1.596 | 8.14E−06 | 2.96E−03 |
| Gsn | 26 | 0.437 | 0.186 | 1.055 | 8.80E−06 | 3.18E−03 |
| Mat2a | 26 | 0.378 | 0.6 | −0.741 | 8.83E−06 | 3.18E−03 |
| Srsf7 | 26 | 0.374 | 0.557 | −0.602 | 9.23E−06 | 3.32E−03 |
| Cd200r3 | 26 | 0.267 | 0.057 | 1.849 | 1.17E−05 | 4.10E−03 |
| Mt1 | 26 | 0.522 | 0.257 | 1.064 | 1.18E−05 | 4.12E−03 |
| Patz1 | 26 | 0.154 | 0.314 | −0.988 | 1.39E−05 | 4.77E−03 |
| Tyrobp | 26 | 0.955 | 0.929 | 0.395 | 1.43E−05 | 4.89E−03 |
| Cbx1 | 26 | 0.184 | 0.329 | −0.828 | 1.51E−05 | 5.12E−03 |
| Ccnd2 | 26 | 0.346 | 0.443 | −0.674 | 1.61E−05 | 5.42E−03 |
| Emd | 26 | 0.206 | 0.314 | −1.086 | 1.61E−05 | 5.43E−03 |
| Rgs1 | 26 | 0.84 | 0.6 | 0.590 | 1.65E−05 | 5.55E−03 |
| Impdh2 | 26 | 0.211 | 0.371 | −0.895 | 1.81E−05 | 6.00E−03 |
| Optn | 26 | 0.213 | 0.029 | 2.198 | 1.98E−05 | 6.48E−03 |
| Opa3 | 26 | 0.089 | 0.257 | −1.429 | 2.12E−05 | 6.88E−03 |
| Mns1 | 26 | 0.338 | 0.143 | 1.231 | 2.32E−05 | 7.41E−03 |
| Itm2b | 26 | 0.987 | 0.971 | 0.287 | 2.44E−05 | 7.76E−03 |
| Adora3 | 26 | 0.431 | 0.186 | 0.909 | 2.50E−05 | 7.90E−03 |
| Pim3 | 26 | 0.145 | 0.371 | −1.317 | 2.62E−05 | 8.21E−03 |
| Tmem9 | 26 | 0.437 | 0.229 | 0.978 | 2.95E−05 | 9.14E−03 |
| Eif3h | 26 | 0.84 | 0.686 | 0.444 | 2.96E−05 | 9.16E−03 |
| 1810058l24Rik | 26 | 0.625 | 0.386 | 0.587 | 3.04E−05 | 9.37E−03 |
| Hspa8 | 26 | 0.953 | 0.843 | 0.375 | 3.06E−05 | 9.43E−03 |
| Alox5ap | 26 | 0.762 | 0.543 | 0.807 | 3.09E−05 | 9.49E−03 |
| Coq10b | 26 | 0.226 | 0.314 | −0.675 | 3.54E−05 | 1.07E−02 |
| Fam174a | 26 | 0.201 | 0.4 | −0.766 | 3.59E−05 | 1.09E−02 |
| Spag9 | 26 | 0.153 | 0.343 | −1.004 | 4.00E−05 | 1.19E−02 |
| Fbxo9 | 26 | 0.144 | 0.171 | −0.681 | 4.12E−05 | 1.22E−02 |
| H3f3a | 26 | 0.885 | 0.9 | −0.310 | 4.35E−05 | 1.28E−02 |
| Ndufa6 | 26 | 0.865 | 0.729 | 0.485 | 4.60E−05 | 1.35E−02 |
| Pik3r6 | 26 | 0.366 | 0.129 | 0.880 | 4.81E−05 | 1.40E−02 |
| Ptp4a1 | 26 | 0.354 | 0.543 | −0.748 | 5.01E−05 | 1.45E−02 |
| Dot1l | 26 | 0.088 | 0.2 | −1.211 | 5.22E−05 | 1.50E−02 |
| Cited4 | 26 | 0.891 | 0.829 | −0.273 | 5.73E−05 | 1.63E−02 |
| F2r | 26 | 0.389 | 0.157 | 1.091 | 6.05E−05 | 1.70E−02 |
| Snrpb | 26 | 0.546 | 0.7 | −0.456 | 6.05E−05 | 1.70E−02 |
| Lif | 26 | 0.174 | 0.014 | 3.225 | 6.63E−05 | 1.84E−02 |
| Nmrk1 | 26 | 0.043 | 0.186 | −1.576 | 6.82E−05 | 1.89E−02 |
| Homer2 | 26 | 0.293 | 0.086 | 1.493 | 6.90E−05 | 1.91E−02 |
| Adgre5 | 26 | 0.304 | 0.1 | 1.018 | 7.15E−05 | 1.96E−02 |
| Ctsc | 26 | 0.701 | 0.471 | 0.858 | 7.46E−05 | 2.04E−02 |
| Ddx5 | 26 | 0.943 | 0.986 | −0.374 | 7.71E−05 | 2.10E−02 |
| Kcnq1ot1 | 26 | 0.101 | 0.243 | −1.171 | 7.75E−05 | 2.11E−02 |
| Zcrb1 | 26 | 0.343 | 0.586 | −0.574 | 8.17E−05 | 2.20E−02 |
| Deptor | 26 | 0.119 | 0.329 | −1.074 | 8.21E−05 | 2.21E−02 |
| Dcaf12 | 26 | 0.143 | 0.357 | −0.920 | 8.27E−05 | 2.22E−02 |
| Myeov2 | 26 | 0.826 | 0.614 | 0.406 | 8.54E−05 | 2.28E−02 |
| Il6 | 26 | 0.356 | 0.114 | 1.578 | 8.97E−05 | 2.38E−02 |
| Tubb2b | 26 | 0.164 | 0.043 | 0.556 | 9.08E−05 | 2.40E−02 |
| Rrp15 | 26 | 0.056 | 0.214 | −1.304 | 9.41E−05 | 2.47E−02 |
| Lmo4 | 26 | 0.407 | 0.571 | −0.744 | 9.80E−05 | 2.56E−02 |
| Creg1 | 26 | 0.699 | 0.514 | 0.693 | 1.02E−04 | 2.64E−02 |
| Cuedc1 | 26 | 0.166 | 0.4 | −0.928 | 1.06E−04 | 2.74E−02 |
| Nucb1 | 26 | 0.396 | 0.171 | 0.953 | 1.16E−04 | 2.95E−02 |
| Zcchc10 | 26 | 0.409 | 0.186 | 1.098 | 1.26E−04 | 3.17E−02 |
| Zfp131 | 26 | 0.106 | 0.271 | −1.115 | 1.27E−04 | 3.21E−02 |
| Smco4 | 26 | 0.629 | 0.386 | 0.580 | 1.28E−04 | 3.21E−02 |
| Nhp2l1 | 26 | 0.382 | 0.514 | −0.624 | 1.28E−04 | 3.22E−02 |
| Snrpd3 | 26 | 0.37 | 0.429 | −0.444 | 1.32E−04 | 3.30E−02 |
| Ifitm3 | 26 | 0.169 | 0.314 | −0.813 | 1.32E−04 | 3.31E−02 |
| Rac1 | 26 | 0.703 | 0.486 | 0.575 | 1.33E−04 | 3.32E−02 |
| Cox4i1 | 26 | 0.948 | 0.886 | 0.320 | 1.35E−04 | 3.37E−02 |
| Plscr1 | 26 | 0.553 | 0.286 | 0.843 | 1.45E−04 | 3.55E−02 |
| Ccdc107 | 26 | 0.202 | 0.3 | −0.711 | 1.45E−04 | 3.56E−02 |
| Zfos1 | 26 | 0.264 | 0.3 | −0.542 | 1.46E−04 | 3.58E−02 |
| Rexo2 | 26 | 0.745 | 0.557 | 0.602 | 1.48E−04 | 3.62E−02 |
| Bhlhe40 | 26 | 0.433 | 0.186 | 1.174 | 1.52E−04 | 3.70E−02 |
| Tsc22d1 | 26 | 0.739 | 0.843 | −0.468 | 1.55E−04 | 3.76E−02 |
| Ctsg | 26 | 0.117 | 0.3 | −0.877 | 1.56E−04 | 3.78E−02 |
| Nfkbid | 26 | 0.664 | 0.386 | 0.823 | 1.56E−04 | 3.78E−02 |
| Actb | 26 | 0.999 | 1 | 0.617 | 1.72E−04 | 4.11E−02 |
| Bcl2l11 | 26 | 0.035 | 0.157 | −1.690 | 1.74E−04 | 4.15E−02 |
| Ecm1 | 26 | 0.301 | 0.086 | 1.528 | 1.81E−04 | 4.29E−02 |
| Serf2 | 26 | 0.979 | 0.971 | 0.319 | 1.86E−04 | 4.39E−02 |
| Dnajc2 | 26 | 0.14 | 0.271 | −0.988 | 1.96E−04 | 4.57E−02 |
| Neat1 | 26 | 0.735 | 0.543 | 0.449 | 1.97E−04 | 4.61E−02 |
| Eif3g | 26 | 0.315 | 0.457 | −0.628 | 1.99E−04 | 4.65E−02 |
| Dusp2 | 27 | 0.396 | 0.152 | 1.356 | 9.49E−05 | 2.49E−02 |
| Btg2 | 3 | 0.826 | 0.969 | −0.826 | 2.71E−113 | 5.25E−108 |
| Junb | 3 | 0.945 | 0.99 | −0.529 | 8.39E−66 | 3.05E−61 |
| Ubb | 3 | 0.96 | 0.994 | −0.435 | 1.78E−62 | 4.32E−58 |
| Dusp1 | 3 | 0.55 | 0.847 | −0.794 | 2.66E−59 | 5.33E−55 |
| Bcl2 | 3 | 0.592 | 0.355 | 0.931 | 2.24E−56 | 4.35E−52 |
| Pnrc1 | 3 | 0.567 | 0.824 | −0.650 | 1.08E−54 | 1.84E−50 |
| Pim1 | 3 | 0.402 | 0.691 | −0.806 | 8.46E−53 | 1.37E−48 |
| Jund | 3 | 0.851 | 0.964 | −0.536 | 1.29E−48 | 1.70E−44 |
| Actg1 | 3 | 0.92 | 0.875 | 0.331 | 1.42E−47 | 1.80E−43 |
| Btg1 | 3 | 0.808 | 0.931 | −0.523 | 2.11E−46 | 2.51E−42 |
| Irf7 | 3 | 0.149 | 0.388 | −1.262 | 8.63E−45 | 9.13E−41 |
| Ppp1r15a | 3 | 0.237 | 0.503 | −0.934 | 2.94E−41 | 2.72E−37 |
| Zfp36l2 | 3 | 0.47 | 0.246 | 0.933 | 1.12E−40 | 9.99E−37 |
| Fosb | 3 | 0.211 | 0.462 | −1.004 | 2.21E−38 | 1.76E−34 |
| Hk2 | 3 | 0.23 | 0.445 | −0.999 | 4.43E−36 | 3.26E−32 |
| Nfkbia | 3 | 0.533 | 0.795 | −0.622 | 9.05E−36 | 6.50E−32 |
| Gadd45b | 3 | 0.264 | 0.518 | −0.881 | 1.36E−35 | 9.65E−32 |
| Klf4 | 3 | 0.133 | 0.346 | −1.042 | 4.12E−34 | 2.70E−30 |
| Dusp2 | 3 | 0.235 | 0.07 | 1.410 | 4.13E−34 | 2.70E−30 |
| Cd69 | 3 | 0.493 | 0.703 | −0.730 | 4.86E−34 | 3.14E−30 |
| Fos | 3 | 0.611 | 0.828 | −0.558 | 1.81E−31 | 1.03E−27 |
| B930036N10Rik | 3 | 0.147 | 0.322 | −1.297 | 7.30E−31 | 4.01E−27 |
| Slc7a6os | 3 | 0.156 | 0.356 | −1.073 | 4.21E−30 | 2.17E−26 |
| Igkc | 3 | 0.993 | 0.99 | 0.322 | 2.50E−28 | 1.20E−24 |
| Tiparp | 3 | 0.039 | 0.182 | −1.581 | 5.71E−28 | 2.62E−24 |
| Eif1 | 3 | 0.99 | 1 | −0.260 | 3.15E−27 | 1.39E−23 |
| Sla | 3 | 0.716 | 0.658 | 0.317 | 1.28E−26 | 5.50E−23 |
| Rsrp1 | 3 | 0.466 | 0.665 | −0.640 | 5.63E−26 | 2.26E−22 |
| Bhlhe40 | 3 | 0.438 | 0.633 | −0.573 | 5.97E−26 | 2.38E−22 |
| Igha | 3 | 0.966 | 0.943 | 0.291 | 2.18E−25 | 8.41E−22 |
| Tgif1 | 3 | 0.254 | 0.444 | −0.724 | 7.91E−25 | 2.93E−21 |
| Prr7 | 3 | 0.076 | 0.223 | −1.236 | 9.69E−25 | 3.57E−21 |
| Klrk1 | 3 | 0.546 | 0.684 | −0.499 | 9.42E−24 | 3.19E−20 |
| Tnfaip3 | 3 | 0.301 | 0.489 | −0.677 | 5.84E−22 | 1.68E−18 |
| Clk1 | 3 | 0.338 | 0.518 | −0.578 | 3.00E−21 | 8.35E−18 |
| Ier2 | 3 | 0.685 | 0.822 | −0.556 | 5.05E−21 | 1.38E−17 |
| Ifrd1 | 3 | 0.183 | 0.357 | −0.754 | 3.81E−20 | 9.81E−17 |
| Txnip | 3 | 0.202 | 0.084 | 1.079 | 9.16E−20 | 2.29E−16 |
| Lpar6 | 3 | 0.24 | 0.401 | −0.676 | 1.77E−19 | 4.30E−16 |
| Uhrf2 | 3 | 0.298 | 0.465 | −0.609 | 6.32E−19 | 1.45E−15 |
| Dok1 | 3 | 0.318 | 0.207 | 0.534 | 1.78E−18 | 3.91E−15 |
| Pabpc1 | 3 | 0.698 | 0.855 | −0.452 | 2.17E−18 | 4.72E−15 |
| Ikzf2 | 3 | 0.438 | 0.561 | −0.451 | 2.44E−18 | 5.29E−15 |
| Ptpn22 | 3 | 0.521 | 0.39 | 0.465 | 2.81E−18 | 6.03E−15 |
| Nfkbid | 3 | 0.191 | 0.371 | −0.763 | 5.78E−18 | 1.21E−14 |
| Iglc1 | 3 | 0.184 | 0.359 | −0.525 | 9.64E−18 | 1.96E−14 |
| Hsp90aa1 | 3 | 0.394 | 0.326 | 0.558 | 7.02E−17 | 1.30E−13 |
| Il22 | 3 | 0.082 | 0.207 | −1.460 | 6.26E−16 | 1.08E−12 |
| Phlda1 | 3 | 0.194 | 0.355 | −0.587 | 1.73E−15 | 2.87E−12 |
| Dusp5 | 3 | 0.48 | 0.644 | −0.437 | 3.57E−15 | 5.74E−12 |
| Cebpb | 3 | 0.518 | 0.409 | 0.434 | 3.87E−15 | 6.20E−12 |
| Nr4a1 | 3 | 0.458 | 0.63 | −0.503 | 4.34E−15 | 6.90E−12 |
| Sqstm1 | 3 | 0.227 | 0.375 | −0.607 | 1.49E−14 | 2.25E−11 |
| Il2rg | 3 | 0.543 | 0.701 | −0.419 | 2.42E−14 | 3.58E−11 |
| Icos | 3 | 0.271 | 0.421 | −0.530 | 6.75E−14 | 9.42E−11 |
| Nfil3 | 3 | 0.067 | 0.176 | −0.989 | 1.01E−13 | 1.37E−10 |
| Gimap9 | 3 | 0.372 | 0.268 | 0.441 | 1.09E−13 | 1.47E−10 |
| Ier5 | 3 | 0.375 | 0.535 | −0.499 | 2.92E−13 | 3.77E−10 |
| Actb | 3 | 0.956 | 0.953 | 0.237 | 5.27E−13 | 6.60E−10 |
| Rgs1 | 3 | 0.597 | 0.74 | −0.469 | 8.80E−13 | 1.09E−09 |
| Hexim1 | 3 | 0.059 | 0.153 | −0.847 | 1.04E−12 | 1.28E−09 |
| Zc3h12a | 3 | 0.138 | 0.256 | −0.676 | 1.93E−12 | 2.31E−09 |
| Cited2 | 3 | 0.137 | 0.264 | −0.735 | 2.07E−12 | 2.47E−09 |
| Serpinb6b | 3 | 0.51 | 0.392 | 0.341 | 4.27E−12 | 4.85E−09 |
| Hspa5 | 3 | 0.399 | 0.54 | −0.437 | 7.57E−11 | 7.43E−08 |
| Pik3r1 | 3 | 0.586 | 0.474 | 0.297 | 1.03E−10 | 9.92E−08 |
| Herpud1 | 3 | 0.135 | 0.253 | −0.667 | 2.34E−10 | 2.14E−07 |
| Fam110a | 3 | 0.101 | 0.199 | −0.786 | 2.47E−10 | 2.25E−07 |
| Csrnp1 | 3 | 0.185 | 0.311 | −0.660 | 2.78E−10 | 2.52E−07 |
| Nfkbiz | 3 | 0.248 | 0.38 | −0.505 | 3.16E−10 | 2.83E−07 |
| Litaf | 3 | 0.314 | 0.213 | 0.440 | 3.41E−10 | 3.04E−07 |
| Asb2 | 3 | 0.402 | 0.563 | −0.590 | 3.70E−10 | 3.26E−07 |
| Sh2d2a | 3 | 0.273 | 0.226 | 0.342 | 4.26E−10 | 3.70E−07 |
| Cish | 3 | 0.264 | 0.189 | 0.423 | 8.15E−10 | 6.87E−07 |
| Ccnl1 | 3 | 0.276 | 0.43 | −0.536 | 1.11E−09 | 9.06E−07 |
| Eef2 | 3 | 0.841 | 0.891 | −0.269 | 1.48E−09 | 1.18E−06 |
| Chd2 | 3 | 0.134 | 0.241 | −0.663 | 1.90E−09 | 1.49E−06 |
| Il2rb | 3 | 0.468 | 0.389 | 0.286 | 3.01E−09 | 2.26E−06 |
| Nrip1 | 3 | 0.379 | 0.492 | −0.314 | 3.68E−09 | 2.71E−06 |
| Hsp90ab1 | 3 | 0.579 | 0.551 | 0.247 | 4.22E−09 | 3.07E−06 |
| H2-Q7 | 3 | 0.183 | 0.299 | −0.562 | 4.85E−09 | 3.50E−06 |
| Tnf | 3 | 0.155 | 0.272 | −0.594 | 5.24E−09 | 3.76E−06 |
| BC031181 | 3 | 0.458 | 0.531 | −0.307 | 6.68E−09 | 4.74E−06 |
| Uqcrh | 3 | 0.733 | 0.699 | 0.168 | 6.75E−09 | 4.78E−06 |
| Ptp4a1 | 3 | 0.188 | 0.286 | −0.533 | 1.15E−08 | 7.84E−06 |
| Ccl4 | 3 | 0.165 | 0.099 | 0.609 | 1.39E−08 | 9.32E−06 |
| Txk | 3 | 0.529 | 0.64 | −0.289 | 1.39E−08 | 9.32E−06 |
| Kdm6b | 3 | 0.234 | 0.346 | −0.450 | 1.48E−08 | 9.88E−06 |
| Slc3a2 | 3 | 0.464 | 0.422 | 0.275 | 1.49E−08 | 9.95E−06 |
| Tnfrsf18 | 3 | 0.378 | 0.306 | 0.282 | 1.85E−08 | 1.22E−05 |
| Peli1 | 3 | 0.155 | 0.256 | −0.536 | 2.62E−08 | 1.68E−05 |
| Rbm3 | 3 | 0.669 | 0.775 | −0.346 | 4.08 E−08 | 2.49E−05 |
| Rasgrp1 | 3 | 0.184 | 0.281 | −0.495 | 4.40E−08 | 2.68E−05 |
| Hbp1 | 3 | 0.083 | 0.155 | −0.641 | 4.76E−08 | 2.88E−05 |
| Pcf11 | 3 | 0.084 | 0.166 | −0.699 | 4.80E−08 | 2.89E−05 |
| Tns4 | 3 | 0.147 | 0.258 | −0.647 | 5.88E−08 | 3.49E−05 |
| Rora | 3 | 0.552 | 0.617 | −0.268 | 6.69E−08 | 3.92E−05 |
| Prdx6 | 3 | 0.719 | 0.67 | 0.153 | 7.69E−08 | 4.47E−05 |
| Gpr65 | 3 | 0.176 | 0.111 | 0.508 | 8.15E−08 | 4.69E−05 |
| Tpm4 | 3 | 0.398 | 0.34 | 0.239 | 9.64E−08 | 5.48E−05 |
| Tnfsf11 | 3 | 0.24 | 0.166 | 0.430 | 1.25E−07 | 7.00E−05 |
| Abi3 | 3 | 0.22 | 0.36 | −0.553 | 1.32E−07 | 7.38E−05 |
| Samsn1 | 3 | 0.449 | 0.381 | 0.225 | 1.35E−07 | 7.51E−05 |
| Oser1 | 3 | 0.273 | 0.379 | −0.404 | 1.52E−07 | 8.39E−05 |
| Gpr171 | 3 | 0.279 | 0.22 | 0.298 | 1.76E−07 | 9.61E−05 |
| Traf1 | 3 | 0.319 | 0.395 | −0.340 | 2.26E−07 | 1.21E−04 |
| Tuba1a | 3 | 0.118 | 0.21 | −0.674 | 2.35E−07 | 1.25E−04 |
| Sla2 | 3 | 0.27 | 0.204 | 0.330 | 3.15E−07 | 1.63E−04 |
| Ccng2 | 3 | 0.12 | 0.205 | −0.558 | 3.26E−07 | 1.68E−04 |
| 5031425E22Rik | 3 | 0.202 | 0.311 | −0.472 | 4.05E−07 | 2.06E−04 |
| Cmas | 3 | 0.196 | 0.305 | −0.539 | 4.14E−07 | 2.10E−04 |
| Lmo4 | 3 | 0.583 | 0.682 | −0.256 | 6.18E−07 | 3.02E−04 |
| Tgoln1 | 3 | 0.222 | 0.334 | −0.444 | 6.36E−07 | 3.09E−04 |
| Erdr1 | 3 | 0.118 | 0.184 | −0.515 | 6.51E−07 | 3.16E−04 |
| Emb | 3 | 0.794 | 0.827 | −0.232 | 6.91E−07 | 3.33E−04 |
| Ubc | 3 | 0.343 | 0.46 | −0.304 | 8.30E−07 | 3.92E−04 |
| Pcbp2 | 3 | 0.491 | 0.633 | −0.325 | 8.47E−07 | 3.99E−04 |
| 4930523C07Rik | 3 | 0.185 | 0.281 | −0.469 | 9.87E−07 | 4.60E−04 |
| Tle3 | 3 | 0.189 | 0.125 | 0.507 | 1.00E−06 | 4.65E−04 |
| Il16 | 3 | 0.087 | 0.161 | −0.633 | 1.30E−06 | 5.87E−04 |
| Sft2d2 | 3 | 0.174 | 0.114 | 0.455 | 1.32E−06 | 5.95E−04 |
| Sepp1 | 3 | 0.849 | 0.899 | −0.270 | 1.33E−06 | 6.00E−04 |
| H2-K1 | 3 | 0.828 | 0.897 | −0.269 | 1.35E−06 | 6.04E−04 |
| Birc3 | 3 | 0.144 | 0.22 | −0.453 | 1.63E−06 | 7.17E−04 |
| Tmem243 | 3 | 0.21 | 0.305 | −0.402 | 1.87E−06 | 8.10E−04 |
| Vps37b | 3 | 0.49 | 0.514 | −0.262 | 2.55E−06 | 1.06E−03 |
| Ptprcap | 3 | 0.625 | 0.568 | 0.175 | 2.86E−06 | 1.17E−03 |
| Serinc3 | 3 | 0.202 | 0.143 | 0.464 | 3.66E−06 | 1.45E−03 |
| Gltscr2 | 3 | 0.468 | 0.572 | −0.269 | 3.86E−06 | 1.52E−03 |
| Rsl24d1 | 3 | 0.164 | 0.111 | 0.429 | 4.20E−06 | 1.64E−03 |
| Maff | 3 | 0.385 | 0.446 | −0.284 | 4.62E−06 | 1.79E−03 |
| Npc2 | 3 | 0.496 | 0.457 | 0.158 | 5.10E−06 | 1.96E−03 |
| Acpp | 3 | 0.093 | 0.162 | −0.600 | 5.45E−06 | 2.08E−03 |
| Ppt2 | 3 | 0.361 | 0.309 | 0.209 | 5.74E−06 | 2.18E−03 |
| Gcnt2 | 3 | 0.13 | 0.203 | −0.416 | 5.76E−06 | 2.18E−03 |
| Pnisr | 3 | 0.213 | 0.314 | −0.367 | 6.48E−06 | 2.42E−03 |
| D16Ertd472e | 3 | 0.136 | 0.223 | −0.475 | 6.49E−06 | 2.43E−03 |
| Gna13 | 3 | 0.265 | 0.348 | −0.321 | 7.31E−06 | 2.70E−03 |
| Socs1 | 3 | 0.271 | 0.231 | 0.281 | 8.07E−06 | 2.94E−03 |
| Ifngr1 | 3 | 0.725 | 0.673 | 0.162 | 1.11E−05 | 3.90E−03 |
| H2-D1 | 3 | 0.979 | 0.99 | −0.188 | 1.20E−05 | 4.17E−03 |
| Hilpda | 3 | 0.404 | 0.527 | −0.321 | 1.25E−05 | 4.32E−03 |
| Fam102a | 3 | 0.219 | 0.169 | 0.330 | 1.33E−05 | 4.57E−03 |
| Crem | 3 | 0.369 | 0.289 | 0.312 | 1.43E−05 | 4.89E−03 |
| Rinl | 3 | 0.465 | 0.388 | 0.248 | 1.45E−05 | 4.95E−03 |
| Spop | 3 | 0.205 | 0.301 | −0.398 | 1.63E−05 | 5.47E−03 |
| Jchain | 3 | 0.781 | 0.732 | 0.156 | 1.70E−05 | 5.67E−03 |
| F2r | 3 | 0.258 | 0.36 | −0.384 | 1.71E−05 | 5.70E−03 |
| Rgs2 | 3 | 0.37 | 0.466 | −0.291 | 1.72E−05 | 5.73E−03 |
| Ahr | 3 | 0.268 | 0.205 | 0.390 | 1.82E−05 | 6.02E−03 |
| Ndufs4 | 3 | 0.293 | 0.238 | 0.222 | 1.98E−05 | 6.48E−03 |
| Hes1 | 3 | 0.093 | 0.176 | −0.774 | 2.01E−05 | 6.56E−03 |
| Ddx18 | 3 | 0.188 | 0.159 | 0.252 | 2.36E−05 | 7.53E−03 |
| Eprs | 3 | 0.127 | 0.202 | −0.476 | 2.38E−05 | 7.59E−03 |
| Rara | 3 | 0.1 | 0.17 | −0.564 | 2.43E−05 | 7.72E−03 |
| Prelid2 | 3 | 0.323 | 0.269 | 0.243 | 3.05E−05 | 9.40E−03 |
| Clcn3 | 3 | 0.202 | 0.277 | −0.363 | 3.20E−05 | 9.81E−03 |
| Tbx21 | 3 | 0.185 | 0.137 | 0.344 | 3.22E−05 | 9.86E−03 |
| Exosc3 | 3 | 0.167 | 0.125 | 0.348 | 3.25E−05 | 9.91E−03 |
| Ppp1r10 | 3 | 0.19 | 0.274 | −0.337 | 3.60E−05 | 1.09E−02 |
| Cbx3 | 3 | 0.276 | 0.238 | 0.168 | 3.94E−05 | 1.18E−02 |
| Rnf19b | 3 | 0.173 | 0.231 | −0.353 | 4.11E−05 | 1.22E−02 |
| Ccdc174 | 3 | 0.11 | 0.167 | −0.475 | 4.11E−05 | 1.22E−02 |
| Gimap5 | 3 | 0.45 | 0.395 | 0.168 | 4.82E−05 | 1.40E−02 |
| Dgat1 | 3 | 0.306 | 0.404 | −0.324 | 4.90E−05 | 1.42E−02 |
| Plk3 | 3 | 0.177 | 0.143 | 0.331 | 5.41E−05 | 1.55E−02 |
| H2-Q4 | 3 | 0.167 | 0.254 | −0.495 | 6.01E−05 | 1.70E−02 |
| Znhit1 | 3 | 0.188 | 0.154 | 0.272 | 6.69E−05 | 1.85E−02 |
| Nop56 | 3 | 0.089 | 0.162 | −0.544 | 7.45E−05 | 2.03E−02 |
| Neurl3 | 3 | 0.314 | 0.382 | −0.256 | 7.70E−05 | 2.10E−02 |
| Il18r1 | 3 | 0.431 | 0.465 | −0.175 | 8.60E−05 | 2.29E−02 |
| Mbd2 | 3 | 0.209 | 0.314 | −0.452 | 8.79E−05 | 2.34E−02 |
| Tubb4b | 3 | 0.138 | 0.233 | −0.547 | 8.85E−05 | 2.35E−02 |
| Dtx3 | 3 | 0.185 | 0.275 | −0.378 | 9.21E−05 | 2.42E−02 |
| Slc38a2 | 3 | 0.12 | 0.186 | −0.469 | 9.33E−05 | 2.45E−02 |
| Ehd1 | 3 | 0.158 | 0.119 | 0.345 | 1.13E−04 | 2.90E−02 |
| Abhd2 | 3 | 0.286 | 0.24 | 0.335 | 1.13E−04 | 2.90E−02 |
| Zc3hav1 | 3 | 0.163 | 0.248 | −0.395 | 1.22E−04 | 3.09E−02 |
| Dtx1 | 3 | 0.173 | 0.251 | −0.367 | 1.22E−04 | 3.09E−02 |
| Nfkb2 | 3 | 0.103 | 0.165 | −0.512 | 1.35E−04 | 3.36E−02 |
| Gem | 3 | 0.463 | 0.509 | −0.304 | 1.38E−04 | 3.42E−02 |
| Bcl2a1d | 3 | 0.344 | 0.394 | −0.236 | 1.43E−04 | 3.53E−02 |
| Xbp1 | 3 | 0.31 | 0.282 | 0.218 | 1.50E−04 | 3.67E−02 |
| Slc16a6 | 3 | 0.233 | 0.309 | −0.365 | 1.52E−04 | 3.69E−02 |
| Cdc42se2 | 3 | 0.198 | 0.283 | −0.363 | 1.53E−04 | 3.72E−02 |
| Clta | 3 | 0.37 | 0.32 | 0.163 | 1.78E−04 | 4.24E−02 |
| Ahcyl2 | 3 | 0.515 | 0.469 | 0.214 | 1.83E−04 | 4.33E−02 |
| Saraf | 3 | 0.394 | 0.493 | −0.258 | 1.85E−04 | 4.36E−02 |
| Tgfb1 | 3 | 0.268 | 0.361 | −0.366 | 2.06E−04 | 4.79E−02 |
| Cnot2 | 3 | 0.089 | 0.154 | −0.535 | 2.10E−04 | 4.87E−02 |
| Ighg1 | 31 | 0.165 | 0 | 4.475 | 1.46E−06 | 6.53E−04 |
| Ifi30 | 31 | 0.417 | 0.274 | 1.031 | 3.76E−06 | 1.48E−03 |
| Cyba | 31 | 0.626 | 0.453 | 0.749 | 1.25E−05 | 4.35E−03 |
| AW112010 | 31 | 0.087 | 0.379 | −0.682 | 1.66E−05 | 5.56E−03 |
| Ppia | 31 | 0.904 | 0.768 | 0.300 | 1.91E−05 | 6.27E−03 |
| Tmem258 | 31 | 0.4 | 0.368 | 0.478 | 4.81E−05 | 1.40E−02 |
| Mif | 31 | 0.461 | 0.232 | 0.781 | 1.24E−04 | 3.14E−02 |
| H2-DMb2 | 31 | 0.617 | 0.421 | 0.572 | 1.73E−04 | 4.14E−02 |
| 1600010M07Rik | 31 | 0.017 | 0.189 | −2.290 | 1.78E−04 | 4.24E−02 |
| Npm1 | 31 | 0.661 | 0.505 | 0.488 | 1.79E−04 | 4.25E−02 |
| Cebpb | 33 | 1 | 0.528 | 2.572 | 8.08E−08 | 4.66E−05 |
| Nr4a1 | 33 | 1 | 0.167 | 3.626 | 5.30E−07 | 2.63E−04 |
| Errfi1 | 33 | 1 | 0.153 | 4.176 | 8.06E−07 | 3.82E−04 |
| Mt1 | 33 | 1 | 0.694 | 3.108 | 1.08E−06 | 5.00E−04 |
| Thbs1 | 33 | 1 | 0.111 | 3.767 | 2.94E−06 | 1.20E−03 |
| Ccl7 | 33 | 0.8 | 0 | 7.280 | 1.34E−05 | 4.62E−03 |
| Mt2 | 33 | 0.8 | 0.347 | 2.965 | 1.81E−05 | 5.99E−03 |
| Hspa8 | 33 | 1 | 0.806 | 0.917 | 5.18E−05 | 1.49E−02 |
| Cyr61 | 33 | 1 | 0.264 | 2.237 | 7.96E−05 | 2.15E−02 |
| Vmp1 | 33 | 1 | 0.389 | 2.516 | 1.37E−04 | 3.40E−02 |
| Mylk | 33 | 0.8 | 0.417 | 1.983 | 1.48E−04 | 3.63E−02 |
| Csrnp1 | 33 | 0.6 | 0.014 | 4.333 | 1.84E−04 | 4.35E−02 |
| Cxcl1 | 33 | 0.6 | 0.014 | 4.780 | 2.16E−04 | 4.98E−02 |
| Klf6 | 34 | 1 | 0.444 | 1.381 | 3.64E−06 | 1.44E−03 |
| Dusp1 | 34 | 1 | 0.944 | 1.553 | 1.30E−05 | 4.49E−03 |
| Jun | 34 | 0.815 | 0.556 | 1.650 | 1.41E−05 | 4.84E−03 |
| Hp | 34 | 0.778 | 0.111 | 2.076 | 2.60E−05 | 8.17E−03 |
| Zfp36 | 34 | 1 | 0.944 | 1.022 | 2.61E−05 | 8.19E−03 |
| Actg1 | 34 | 1 | 1 | 0.491 | 3.69E−05 | 1.11E−02 |
| Osm | 34 | 0.63 | 0.111 | 3.025 | 6.22E−05 | 1.74E−02 |
| 2700029M09Rik | 34 | 0.111 | 0.722 | −1.619 | 7.03E−05 | 1.94E−02 |
| Ctsd | 34 | 0.593 | 0.611 | 1.128 | 8.03E−05 | 2.17E−02 |
| Oas1a | 34 | 0 | 0.333 | −4.376 | 1.99E−04 | 4.65E−02 |
| Pkp3 | 34 | 0.296 | 0 | 4.344 | 2.09E−04 | 4.84E−02 |
| Dusp1 | 36 | 0.981 | 0.944 | 1.061 | 1.05E−09 | 8.69E−07 |
| Cxcl2 | 36 | 0.879 | 0.5 | 2.134 | 2.52E−08 | 1.62E−05 |
| Zfp36 | 36 | 0.873 | 0.444 | 1.362 | 4.84E−07 | 2.42E−04 |
| Btg1 | 36 | 0.943 | 0.944 | −0.853 | 7.36E−06 | 2.71E−03 |
| Junb | 36 | 0.994 | 0.889 | 0.761 | 1.05E−05 | 3.70E−03 |
| Fos | 36 | 0.815 | 0.556 | 0.893 | 3.16E−05 | 9.69E−03 |
| Ccr12 | 36 | 0.924 | 0.556 | 0.948 | 3.33E−05 | 1.01E−02 |
| Osm | 36 | 0.554 | 0.056 | 3.512 | 4.40E−05 | 1.30E−02 |
| Psmb3 | 36 | 0.076 | 0.5 | −1.766 | 6.03E−05 | 1.70E−02 |
| Plac8 | 36 | 0.083 | 0.5 | −2.336 | 7.78E−05 | 2.11E−02 |
| Laptm5 | 36 | 0.248 | 0.778 | −1.097 | 1.30E−04 | 3.25E−02 |
| Nfkbia | 36 | 0.86 | 0.556 | 1.136 | 1.79E−04 | 4.25E−02 |
| Nfkbiz | 36 | 0.605 | 0.167 | 1.880 | 2.08E−04 | 4.84E−02 |
| Sla | 4 | 0.794 | 0.538 | 0.969 | 2.51E−32 | 1.48E−28 |
| Hspa1a | 4 | 0.242 | 0.016 | 3.237 | 3.38E−32 | 1.97E−28 |
| Vps37b | 4 | 0.458 | 0.761 | −0.984 | 1.98E−30 | 1.04E−26 |
| Ifrd1 | 4 | 0.334 | 0.632 | −1.143 | 7.30E−29 | 3.60E−25 |
| Jund | 4 | 0.892 | 0.983 | −0.446 | 6.06E−28 | 2.75E−24 |
| Junb | 4 | 0.974 | 1 | −0.294 | 1.32E−23 | 4.41E−20 |
| Fosb | 4 | 0.434 | 0.725 | −0.828 | 1.90E−22 | 5.73E−19 |
| Crem | 4 | 0.324 | 0.608 | −1.068 | 2.14E−21 | 5.98E−18 |
| Ighg1 | 4 | 0.152 | 0.009 | 2.835 | 6.74E−20 | 1.70E−16 |
| Pim1 | 4 | 0.555 | 0.787 | −0.650 | 9.41E−20 | 2.34E−16 |
| Maff | 4 | 0.53 | 0.798 | −0.559 | 2.54E−19 | 6.10E−16 |
| Eif1 | 4 | 0.995 | 0.997 | −0.261 | 5.38E−19 | 1.25E−15 |
| Bcl2 | 4 | 0.83 | 0.631 | 0.595 | 9.99E−19 | 2.24E−15 |
| Klf4 | 4 | 0.249 | 0.528 | −0.915 | 8.57E−18 | 1.76E−14 |
| Hsp90aa1 | 4 | 0.591 | 0.434 | 0.823 | 9.70E−18 | 1.97E−14 |
| Ubc | 4 | 0.352 | 0.611 | −0.751 | 1.24E−17 | 2.51E−14 |
| Emb | 4 | 0.625 | 0.777 | −0.562 | 1.30E−17 | 2.60E−14 |
| Tnfaip3 | 4 | 0.452 | 0.711 | −0.653 | 5.35E−17 | 1.01E−13 |
| Prr7 | 4 | 0.129 | 0.368 | −1.231 | 6.59E−17 | 1.23E−13 |
| Ncoa7 | 4 | 0.717 | 0.495 | 0.664 | 1.73E−16 | 3.10E−13 |
| Lmna | 4 | 0.198 | 0.422 | −1.142 | 5.25E−16 | 9.14E−13 |
| Fam110a | 4 | 0.308 | 0.526 | −0.910 | 8.04E−16 | 1.37E−12 |
| Cwc25 | 4 | 0.082 | 0.286 | −1.528 | 1.08E−15 | 1.80E−12 |
| Bhlhe40 | 4 | 0.555 | 0.761 | −0.529 | 2.89E−15 | 4.70E−12 |
| Igkc | 4 | 0.992 | 0.993 | 0.522 | 1.54E−14 | 2.32E−11 |
| Rasl11a | 4 | 0.563 | 0.34 | 0.883 | 2.96E−14 | 4.33E−11 |
| Hsp90ab1 | 4 | 0.71 | 0.639 | 0.576 | 4.53E−14 | 6.42E−11 |
| Tgif1 | 4 | 0.442 | 0.66 | −0.551 | 1.85E−13 | 2.45E−10 |
| Pnrc1 | 4 | 0.73 | 0.855 | −0.435 | 7.84E−13 | 9.73E−10 |
| Sqstm1 | 4 | 0.365 | 0.566 | −0.648 | 1.97E−12 | 2.35E−09 |
| Dnajb1 | 4 | 0.316 | 0.152 | 1.254 | 2.45E−12 | 2.87E−09 |
| Ptpn22 | 4 | 0.437 | 0.251 | 0.859 | 4.74E−12 | 5.38E−09 |
| Ccng2 | 4 | 0.239 | 0.463 | −0.679 | 4.86E−12 | 5.50E−09 |
| Zc3h12a | 4 | 0.27 | 0.479 | −0.761 | 7.63E−12 | 8.41E−09 |
| Rilpl2 | 4 | 0.237 | 0.077 | 1.286 | 9.02E−12 | 9.85E−09 |
| Nabp1 | 4 | 0.357 | 0.568 | −0.645 | 1.97E−11 | 2.08E−08 |
| Ube2s | 4 | 0.432 | 0.648 | −0.485 | 3.44E−11 | 3.54E−08 |
| Srsf2 | 4 | 0.527 | 0.62 | −0.468 | 3.68E−11 | 3.77E−08 |
| Atf3 | 4 | 0.113 | 0.291 | −1.066 | 4.69E−11 | 4.72E−08 |
| Phlda1 | 4 | 0.288 | 0.507 | −0.547 | 5.57E−11 | 5.57E−08 |
| Btg2 | 4 | 0.835 | 0.958 | −0.231 | 7.48E−11 | 7.37E−08 |
| Saraf | 4 | 0.432 | 0.622 | −0.530 | 7.86E−11 | 7.68E−08 |
| Gimap1 | 4 | 0.56 | 0.343 | 0.584 | 1.23E−10 | 1.17E−07 |
| Hspe1 | 4 | 0.512 | 0.362 | 0.670 | 1.77E−10 | 1.65E−07 |
| Coq10b | 4 | 0.298 | 0.497 | −0.674 | 2.49E−10 | 2.27E−07 |
| Hspa8 | 4 | 0.861 | 0.831 | 0.431 | 5.85E−10 | 5.00E−07 |
| Gna13 | 4 | 0.254 | 0.443 | −0.674 | 6.56E−10 | 5.56E−07 |
| H2-D1 | 4 | 0.977 | 0.99 | −0.231 | 8.63E−10 | 7.27E−07 |
| Tcrg-C1 | 4 | 0.326 | 0.136 | 1.027 | 1.08E−09 | 8.87E−07 |
| Uhrf2 | 4 | 0.545 | 0.706 | −0.558 | 1.17E−09 | 9.57E−07 |
| Dot1l | 4 | 0.054 | 0.181 | −1.046 | 1.19E−09 | 9.71E−07 |
| Srgn | 4 | 0.738 | 0.617 | 0.492 | 1.38E−09 | 1.10E−06 |
| Dusp1 | 4 | 0.71 | 0.841 | −0.424 | 3.19E−09 | 2.38E−06 |
| Ptp4a1 | 4 | 0.265 | 0.43 | −0.687 | 3.62E−09 | 2.68E−06 |
| 4930523C07Rik | 4 | 0.365 | 0.51 | −0.605 | 8.93E−09 | 6.17E−06 |
| Gltscr2 | 4 | 0.524 | 0.659 | −0.495 | 1.16E−08 | 7.84E−06 |
| Rbm3 | 4 | 0.635 | 0.754 | −0.399 | 1.40E−08 | 9.37E−06 |
| Emd | 4 | 0.244 | 0.409 | −0.653 | 1.55E−08 | 1.03E−05 |
| Dgat1 | 4 | 0.491 | 0.66 | −0.450 | 1.66E−08 | 1.10E−05 |
| Neurl3 | 4 | 0.26 | 0.448 | −0.600 | 2.15E−08 | 1.40E−05 |
| Gpx1 | 4 | 0.763 | 0.76 | −0.322 | 2.94E−08 | 1.86E−05 |
| Gimap5 | 4 | 0.409 | 0.232 | 0.701 | 3.55E−08 | 2.21E−05 |
| Mmd | 4 | 0.35 | 0.19 | 0.817 | 4.03E−08 | 2.47E−05 |
| Neat1 | 4 | 0.386 | 0.289 | 0.600 | 4.81E−08 | 2.90E−05 |
| Hspa5 | 4 | 0.568 | 0.69 | −0.402 | 7.66E−08 | 4.46E−05 |
| Gpr183 | 4 | 0.357 | 0.51 | −0.523 | 9.52E−08 | 5.42E−05 |
| Ppp1r15a | 4 | 0.329 | 0.493 | −0.513 | 1.21E−07 | 6.84E−05 |
| Tiparp | 4 | 0.049 | 0.153 | −1.235 | 1.28E−07 | 7.14E−05 |
| Il2rg | 4 | 0.64 | 0.791 | −0.301 | 1.60E−07 | 8.79E−05 |
| Il4ra | 4 | 0.069 | 0.193 | −1.110 | 1.75E−07 | 9.59E−05 |
| Arg1 | 4 | 0.28 | 0.138 | 1.029 | 2.02E−07 | 1.09E−04 |
| Hk2 | 4 | 0.226 | 0.378 | −0.726 | 2.03E−07 | 1.10E−04 |
| B2m | 4 | 0.992 | 0.986 | −0.174 | 2.28E−07 | 1.22E−04 |
| Pop4 | 4 | 0.051 | 0.16 | −1.155 | 3.07E−07 | 1.60E−04 |
| Ahr | 4 | 0.211 | 0.099 | 0.979 | 3.29E−07 | 1.70E−04 |
| Cish | 4 | 0.221 | 0.092 | 1.037 | 4.61E−07 | 2.31E−04 |
| Gpr132 | 4 | 0.56 | 0.726 | −0.351 | 4.68E−07 | 2.35E−04 |
| Nfkbia | 4 | 0.743 | 0.866 | −0.242 | 5.88E−07 | 2.89E−04 |
| Atf4 | 4 | 0.347 | 0.47 | −0.529 | 6.24E−07 | 3.05E−04 |
| Arf4 | 4 | 0.476 | 0.571 | −0.400 | 7.76E−07 | 3.69E−04 |
| Tnfsf11 | 4 | 0.424 | 0.253 | 0.445 | 8.09E−07 | 3.83E−04 |
| Rsrp1 | 4 | 0.414 | 0.551 | −0.470 | 8.58E−07 | 4.04E−04 |
| Cdk11b | 4 | 0.149 | 0.284 | −0.771 | 9.01E−07 | 4.22E−04 |
| P2ry10 | 4 | 0.262 | 0.392 | −0.583 | 1.12E−06 | 5.15E−04 |
| Odc1 | 4 | 0.566 | 0.652 | −0.516 | 1.53E−06 | 6.77E−04 |
| Zfp36l2 | 4 | 0.419 | 0.279 | 0.651 | 1.53E−06 | 6.77E−04 |
| BC031181 | 4 | 0.409 | 0.533 | −0.477 | 1.90E−06 | 8.21E−04 |
| Birc3 | 4 | 0.262 | 0.411 | −0.586 | 2.37E−06 | 9.98E−04 |
| Tgoln1 | 4 | 0.211 | 0.341 | −0.597 | 2.82E−06 | 1.16E−03 |
| Ccdc71l | 4 | 0.144 | 0.267 | −0.784 | 3.29E−06 | 1.32E−03 |
| Rora | 4 | 0.625 | 0.721 | −0.327 | 3.67E−06 | 1.45E−03 |
| Isy1 | 4 | 0.229 | 0.382 | −0.531 | 3.74E−06 | 1.47E−03 |
| Ubald2 | 4 | 0.219 | 0.364 | −0.679 | 4.20E−06 | 1.64E−03 |
| DIl1 | 4 | 0.059 | 0.153 | −1.050 | 4.61E−06 | 1.79E−03 |
| Dph3 | 4 | 0.203 | 0.345 | −0.586 | 4.80E−06 | 1.86E−03 |
| Rel | 4 | 0.085 | 0.176 | −0.907 | 5.09E−06 | 1.95E−03 |
| Zfp36l1 | 4 | 0.913 | 0.848 | 0.290 | 5.89E−06 | 2.23E−03 |
| Cited2 | 4 | 0.38 | 0.516 | −0.524 | 5.89E−06 | 2.23E−03 |
| Egr1 | 4 | 0.419 | 0.274 | 0.472 | 6.47E−06 | 2.42E−03 |
| Eif4a1 | 4 | 0.694 | 0.749 | −0.248 | 6.77E−06 | 2.52E−03 |
| Gem | 4 | 0.753 | 0.815 | −0.384 | 7.89E−06 | 2.89E−03 |
| Dusp5 | 4 | 0.36 | 0.505 | −0.399 | 8.25E−06 | 3.00E−03 |
| Serp1 | 4 | 0.445 | 0.566 | −0.379 | 8.38E−06 | 3.04E−03 |
| Herpud1 | 4 | 0.229 | 0.366 | −0.605 | 8.85E−06 | 3.19E−03 |
| Arf6 | 4 | 0.53 | 0.375 | 0.442 | 9.80E−06 | 3.50E−03 |
| Ccnl1 | 4 | 0.285 | 0.409 | −0.529 | 1.02E−05 | 3.61E−03 |
| Nfkbiz | 4 | 0.267 | 0.401 | −0.538 | 1.04E−05 | 3.69E−03 |
| Kdm6b | 4 | 0.398 | 0.561 | −0.392 | 1.04E−05 | 3.69E−03 |
| Txk | 4 | 0.324 | 0.441 | −0.489 | 1.05E−05 | 3.73E−03 |
| Eprs | 4 | 0.141 | 0.26 | −0.700 | 1.19E−05 | 4.13E−03 |
| Dnajb2 | 4 | 0.069 | 0.166 | −0.778 | 1.44E−05 | 4.93E−03 |
| H2-K1 | 4 | 0.835 | 0.862 | −0.192 | 1.49E−05 | 5.05E−03 |
| Cisd2 | 4 | 0.229 | 0.354 | −0.517 | 1.49E−05 | 5.06E−03 |
| Nfkbie | 4 | 0.159 | 0.239 | −0.658 | 1.73E−05 | 5.76E−03 |
| Snx18 | 4 | 0.129 | 0.246 | −0.657 | 1.77E−05 | 5.88E−03 |
| Ubald1 | 4 | 0.111 | 0.226 | −0.735 | 1.79E−05 | 5.93E−03 |
| Ramp3 | 4 | 0.201 | 0.329 | −0.598 | 1.91E−05 | 6.27E−03 |
| Nfil3 | 4 | 0.09 | 0.188 | −0.714 | 2.40E−05 | 7.64E−03 |
| Srsf5 | 4 | 0.697 | 0.774 | −0.284 | 2.47E−05 | 7.82E−03 |
| Icos | 4 | 0.113 | 0.225 | −0.789 | 2.60E−05 | 8.17E−03 |
| Nr4a3 | 4 | 0.272 | 0.169 | 0.743 | 3.17E−05 | 9.73E−03 |
| Slc3a2 | 4 | 0.404 | 0.348 | 0.374 | 3.38E−05 | 1.03E−02 |
| Cdc14a | 4 | 0.188 | 0.307 | −0.494 | 3.76E−05 | 1.13E−02 |
| Skil | 4 | 0.247 | 0.376 | −0.507 | 3.88E−05 | 1.16E−02 |
| Klf2 | 4 | 0.388 | 0.423 | 0.325 | 3.94E−05 | 1.18E−02 |
| Igha | 4 | 0.964 | 0.963 | 0.253 | 4.00E−05 | 1.19E−02 |
| Tpm4 | 4 | 0.332 | 0.465 | −0.421 | 4.18E−05 | 1.24E−02 |
| H2-Q4 | 4 | 0.17 | 0.282 | −0.538 | 4.29E−05 | 1.27E−02 |
| Naca | 4 | 0.859 | 0.782 | 0.210 | 4.60E−05 | 1.35E−02 |
| Arpc5l | 4 | 0.221 | 0.105 | 0.710 | 5.25E−05 | 1.50E−02 |
| Npc2 | 4 | 0.44 | 0.315 | 0.459 | 6.46E−05 | 1.80E−02 |
| Arpc3 | 4 | 0.602 | 0.714 | −0.251 | 6.62E−05 | 1.84E−02 |
| Pcf11 | 4 | 0.067 | 0.153 | −0.884 | 6.67E−05 | 1.85E−02 |
| Lcp1 | 4 | 0.62 | 0.54 | 0.343 | 6.96E−05 | 1.92E−02 |
| Gimap6 | 4 | 0.53 | 0.392 | 0.399 | 7.00E−05 | 1.93E−02 |
| Sub1 | 4 | 0.823 | 0.848 | −0.221 | 7.87E−05 | 2.13E−02 |
| Pla2g16 | 4 | 0.254 | 0.138 | 0.633 | 8.15E−05 | 2.19E−02 |
| Actb | 4 | 0.964 | 0.934 | 0.449 | 8.19E−05 | 2.20E−02 |
| Arih1 | 4 | 0.105 | 0.207 | −0.641 | 8.87E−05 | 2.35E−02 |
| Il22 | 4 | 0.252 | 0.378 | −0.485 | 8.97E−05 | 2.38E−02 |
| Ndufa8 | 4 | 0.373 | 0.251 | 0.299 | 9.70E−05 | 2.53E−02 |
| Dad1 | 4 | 0.635 | 0.718 | −0.325 | 9.83E−05 | 2.57E−02 |
| Rinl | 4 | 0.257 | 0.153 | 0.589 | 1.07E−04 | 2.76E−02 |
| Clcn3 | 4 | 0.26 | 0.385 | −0.467 | 1.16E−04 | 2.96E−02 |
| D8Ertd738e | 4 | 0.586 | 0.671 | −0.294 | 1.24E−04 | 3.14E−02 |
| Dok1 | 4 | 0.239 | 0.122 | 0.629 | 1.27E−04 | 3.20E−02 |
| Opa3 | 4 | 0.077 | 0.164 | −0.810 | 1.33E−04 | 3.33E−02 |
| Dnajb9 | 4 | 0.157 | 0.254 | −0.601 | 1.34E−04 | 3.36E−02 |
| Dnaja2 | 4 | 0.267 | 0.153 | 0.596 | 1.35E−04 | 3.37E−02 |
| Ffar2 | 4 | 0.437 | 0.315 | 0.472 | 1.36E−04 | 3.38E−02 |
| Tmem59 | 4 | 0.707 | 0.571 | 0.166 | 1.46E−04 | 3.58E−02 |
| Sdhaf1 | 4 | 0.362 | 0.226 | 0.490 | 1.50E−04 | 3.67E−02 |
| Avpi1 | 4 | 0.18 | 0.272 | −0.679 | 1.57E−04 | 3.80E−02 |
| Tuba4a | 4 | 0.1 | 0.192 | −0.704 | 1.59E−04 | 3.84E−02 |
| Traf1 | 4 | 0.432 | 0.537 | −0.366 | 1.78E−04 | 4.23E−02 |
| Dnajb6 | 4 | 0.306 | 0.383 | −0.357 | 2.12E−04 | 4.92E−02 |
| Ccl7 | 40 | 0.482 | 0.085 | 3.479 | 1.71E−08 | 1.14E−05 |
| Ier2 | 40 | 0.643 | 0.271 | 1.132 | 2.10E−05 | 6.82E−03 |
| Lat | 42 | 0.7 | 0.556 | −0.552 | 2.84E−05 | 8.83E−03 |
| Atp5c1 | 42 | 0.7 | 0.111 | 1.086 | 1.39E−04 | 3.44E−02 |
| Zfp36l1 | 42 | 0.2 | 0.778 | −0.292 | 2.14E−04 | 4.95E−02 |
| Agpat5 | 5 | 0 | 0.438 | −4.897 | 1.57E−04 | 3.80E−02 |
| Mgat2 | 5 | 0 | 0.562 | −5.227 | 2.14E−04 | 4.94E−02 |
| Jun | 6 | 0.666 | 0.338 | 1.583 | 5.37E−85 | 3.47E−80 |
| Zfp36l2 | 6 | 0.719 | 0.485 | 0.974 | 3.81E−52 | 5.83E−48 |
| Fos | 6 | 0.812 | 0.724 | 0.888 | 1.26E−49 | 1.88E−45 |
| Neat1 | 6 | 0.778 | 0.712 | 0.716 | 2.00E−49 | 2.91E−45 |
| Irf7 | 6 | 0.4 | 0.709 | −1.088 | 1.22E−48 | 1.65E−44 |
| Ppia | 6 | 0.993 | 0.982 | 0.247 | 1.69E−35 | 1.18E−31 |
| Btg1 | 6 | 0.965 | 0.987 | −0.414 | 8.69E−33 | 5.32E−29 |
| Rgs1 | 6 | 0.38 | 0.159 | 1.314 | 8.93E−33 | 5.41E−29 |
| Zfp36 | 6 | 0.765 | 0.682 | 0.586 | 4.85E−27 | 2.12E−23 |
| Ighm | 6 | 0.948 | 0.976 | −0.299 | 4.43E−25 | 1.68E−21 |
| Ly6c1 | 6 | 0.938 | 0.947 | −0.406 | 9.16E−24 | 3.12E−20 |
| Klf6 | 6 | 0.442 | 0.284 | 0.903 | 1.27E−23 | 4.25E−20 |
| Tsc22d3 | 6 | 0.592 | 0.448 | 0.555 | 7.80E−22 | 2.24E−18 |
| Klf2 | 6 | 0.681 | 0.661 | 0.739 | 1.16E−21 | 3.28E−18 |
| Ifi27l2a | 6 | 0.625 | 0.805 | −0.683 | 4.66E−21 | 1.29E−17 |
| Cxcr4 | 6 | 0.447 | 0.272 | 0.755 | 1.66E−20 | 4.41E−17 |
| Ly6c2 | 6 | 0.97 | 0.984 | −0.405 | 3.35E−20 | 8.66E−17 |
| Ddit4 | 6 | 0.188 | 0.055 | 1.655 | 1.08E−19 | 2.66E−16 |
| Hist1h1c | 6 | 0.324 | 0.184 | 1.030 | 2.84E−19 | 6.80E−16 |
| Iglc1 | 6 | 0.309 | 0.57 | −0.411 | 8.99E−19 | 2.03E−15 |
| Jund | 6 | 0.95 | 0.949 | 0.470 | 1.58E−17 | 3.13E−14 |
| Fth1 | 6 | 1 | 1 | −0.285 | 7.02E−14 | 9.75E−11 |
| D13Ertd608e | 6 | 0.654 | 0.585 | 0.320 | 7.46E−14 | 1.03E−10 |
| Traf4 | 6 | 0.361 | 0.594 | −0.613 | 8.63E−14 | 1.19E−10 |
| Rpn2 | 6 | 0.486 | 0.395 | 0.495 | 1.04E−13 | 1.41E−10 |
| Klk1 | 6 | 0.904 | 0.885 | 0.304 | 1.12E−13 | 1.51E−10 |
| Psap | 6 | 0.995 | 0.985 | 0.159 | 5.12E−13 | 6.43E−10 |
| Evi2a | 6 | 0.57 | 0.722 | −0.349 | 1.57E−12 | 1.90E−09 |
| Dusp1 | 6 | 0.382 | 0.262 | 0.853 | 2.33E−12 | 2.75E−09 |
| Fyn | 6 | 0.661 | 0.756 | −0.315 | 4.81E−12 | 5.44E−09 |
| Arpc2 | 6 | 0.874 | 0.928 | −0.235 | 9.26E−12 | 1.01E−08 |
| Ccl4 | 6 | 0.849 | 0.883 | 0.430 | 1.13E−11 | 1.22E−08 |
| Trp53i11 | 6 | 0.232 | 0.436 | −0.700 | 2.70E−11 | 2.82E−08 |
| Runx3 | 6 | 0.424 | 0.621 | −0.562 | 5.41E−11 | 5.42E−08 |
| Ctsl | 6 | 0.835 | 0.787 | 0.176 | 1.09E−10 | 1.04E−07 |
| Tmem229b | 6 | 0.43 | 0.605 | −0.472 | 1.18E−10 | 1.12E−07 |
| Pim1 | 6 | 0.282 | 0.506 | −0.542 | 1.22E−10 | 1.16E−07 |
| Hspa8 | 6 | 0.946 | 0.932 | 0.171 | 1.42E−10 | 1.35E−07 |
| Cd69 | 6 | 0.471 | 0.373 | 0.538 | 1.47E−10 | 1.38E−07 |
| Ubb | 6 | 0.985 | 0.968 | 0.172 | 1.48E−10 | 1.39E−07 |
| Rhob | 6 | 0.19 | 0.113 | 0.790 | 1.60E−10 | 1.50E−07 |
| Csrnp1 | 6 | 0.187 | 0.369 | −0.676 | 1.70E−10 | 1.59E−07 |
| Orai1 | 6 | 0.151 | 0.342 | −0.812 | 2.91E−10 | 2.63E−07 |
| Ifi44l | 6 | 0.069 | 0.19 | −0.991 | 3.23E−10 | 2.89E−07 |
| Ifi44 | 6 | 0.087 | 0.213 | −0.834 | 3.33E−10 | 2.97E−07 |
| Ywhah | 6 | 0.471 | 0.385 | 0.552 | 5.01E−10 | 4.34E−07 |
| Igkv1-135 | 6 | 0.035 | 0.195 | −1.520 | 6.19E−10 | 5.27E−07 |
| Pltp | 6 | 0.65 | 0.603 | 0.235 | 1.02E−09 | 8.42E−07 |
| Cd7 | 6 | 0.892 | 0.944 | −0.229 | 1.25E−09 | 1.01E−06 |
| Tagln2 | 6 | 0.901 | 0.869 | 0.179 | 1.93E−09 | 1.51E−06 |
| Vps37b | 6 | 0.218 | 0.434 | −0.767 | 2.91E−09 | 2.20E−06 |
| Cdkn1a | 6 | 0.071 | 0.186 | −1.202 | 5.78E−09 | 4.13E−06 |
| Bcl2l11 | 6 | 0.208 | 0.416 | −0.809 | 9.18E−09 | 6.32E−06 |
| Nfil3 | 6 | 0.076 | 0.22 | −1.017 | 1.01E−08 | 6.88E−06 |
| Adh5 | 6 | 0.232 | 0.169 | 0.390 | 1.93E−08 | 1.27E−05 |
| Atp5c1 | 6 | 0.778 | 0.711 | 0.220 | 2.02E−08 | 1.32E−05 |
| Arpc1b | 6 | 0.919 | 0.944 | −0.187 | 2.28E−08 | 1.47E−05 |
| Cd37 | 6 | 0.649 | 0.597 | 0.256 | 2.90E−08 | 1.84E−05 |
| Litaf | 6 | 0.543 | 0.651 | −0.348 | 3.37E−08 | 2.11E−05 |
| H2-D1 | 6 | 0.916 | 0.957 | −0.222 | 3.39E−08 | 2.12E−05 |
| Irf2bp2 | 6 | 0.582 | 0.477 | 0.313 | 3.57E−08 | 2.22E−05 |
| Gapdh | 6 | 0.645 | 0.627 | 0.154 | 6.22E−08 | 3.67E−05 |
| Pmepa1 | 6 | 0.163 | 0.303 | −0.703 | 7.88E−08 | 4.56E−05 |
| Clec10a | 6 | 0.378 | 0.313 | 0.391 | 2.31E−07 | 1.23E−04 |
| Insig1 | 6 | 0.297 | 0.447 | −0.375 | 3.16E−07 | 1.64E−04 |
| Srsf5 | 6 | 0.802 | 0.86 | −0.323 | 3.48E−07 | 1.78E−04 |
| Btg2 | 6 | 0.526 | 0.697 | −0.306 | 3.82E−07 | 1.95E−04 |
| Gapt | 6 | 0.397 | 0.553 | −0.390 | 4.28E−07 | 2.16E−04 |
| Calm1 | 6 | 0.85 | 0.916 | −0.186 | 5.39E−07 | 2.67E−04 |
| Stat3 | 6 | 0.264 | 0.458 | −0.611 | 5.43E−07 | 2.69E−04 |
| Ly6e | 6 | 0.615 | 0.736 | −0.388 | 5.55E−07 | 2.74E−04 |
| Ctbp1 | 6 | 0.486 | 0.63 | −0.297 | 6.38E−07 | 3.10E−04 |
| Ptprcap | 6 | 0.768 | 0.724 | 0.170 | 6.73E−07 | 3.26E−04 |
| Napsa | 6 | 0.617 | 0.59 | 0.229 | 6.76E−07 | 3.26E−04 |
| Eif4a2 | 6 | 0.635 | 0.572 | 0.262 | 7.76E−07 | 3.69E−04 |
| Igkc | 6 | 1 | 0.998 | −0.201 | 8.90E−07 | 4.17E−04 |
| Ier5 | 6 | 0.805 | 0.794 | 0.219 | 9.26E−07 | 4.33E−04 |
| Csf2rb | 6 | 0.296 | 0.46 | −0.553 | 1.10E−06 | 5.07E−04 |
| Ube2d2a | 6 | 0.534 | 0.673 | −0.304 | 1.30E−06 | 5.87E−04 |
| Eif3i | 6 | 0.472 | 0.426 | 0.223 | 1.31E−06 | 5.92E−04 |
| Ube2d3 | 6 | 0.724 | 0.825 | −0.202 | 1.34E−06 | 6.02E−04 |
| Arid5a | 6 | 0.328 | 0.494 | −0.472 | 1.49E−06 | 6.62E−04 |
| Ptpre | 6 | 0.232 | 0.402 | −0.567 | 1.58E−06 | 7.00E−04 |
| Ablim1 | 6 | 0.166 | 0.335 | −0.751 | 1.95E−06 | 8.37E−04 |
| Prdx2 | 6 | 0.257 | 0.195 | 0.424 | 1.95E−06 | 8.37E−04 |
| Spib | 6 | 0.659 | 0.74 | −0.270 | 1.96E−06 | 8.40E−04 |
| Klk1b27 | 6 | 0.892 | 0.866 | 0.152 | 2.01E−06 | 8.61E−04 |
| Cope | 6 | 0.624 | 0.562 | 0.269 | 2.65E−06 | 1.10E−03 |
| Cbfa2t3 | 6 | 0.18 | 0.314 | −0.651 | 2.90E−06 | 1.19E−03 |
| Sub1 | 6 | 0.95 | 0.965 | −0.153 | 2.94E−06 | 1.20E−03 |
| Myl6 | 6 | 0.812 | 0.786 | 0.172 | 3.10E−06 | 1.26E−03 |
| Cct7 | 6 | 0.447 | 0.386 | 0.273 | 3.41E−06 | 1.37E−03 |
| Sla2 | 6 | 0.59 | 0.525 | 0.287 | 3.45E−06 | 1.38E−03 |
| Atf4 | 6 | 0.311 | 0.494 | −0.601 | 3.66E−06 | 1.45E−03 |
| S100a6 | 6 | 0.617 | 0.762 | −0.324 | 4.09E−06 | 1.60E−03 |
| Ctsd | 6 | 0.361 | 0.299 | 0.336 | 4.33E−06 | 1.68E−03 |
| Ptpn1 | 6 | 0.466 | 0.641 | −0.390 | 4.98E−06 | 1.92E−03 |
| Tmem251 | 6 | 0.158 | 0.284 | −0.547 | 5.24E−06 | 2.01E−03 |
| Gnas | 6 | 0.951 | 0.972 | −0.216 | 5.32E−06 | 2.03E−03 |
| Batf | 6 | 0.153 | 0.312 | −0.708 | 5.59E−06 | 2.12E−03 |
| Kmt2e | 6 | 0.351 | 0.497 | −0.416 | 6.45E−06 | 2.42E−03 |
| Sepw1 | 6 | 0.776 | 0.855 | −0.216 | 7.12E−06 | 2.64E−03 |
| Cd8a | 6 | 0.255 | 0.191 | 0.381 | 7.22E−06 | 2.67E−03 |
| Cd48 | 6 | 0.444 | 0.431 | 0.240 | 1.06E−05 | 3.74E−03 |
| Me2 | 6 | 0.21 | 0.357 | −0.468 | 1.18E−05 | 4.13E−03 |
| Hsp90aa1 | 6 | 0.479 | 0.506 | 0.175 | 1.38E−05 | 4.72E−03 |
| Fam46c | 6 | 0.139 | 0.287 | −0.730 | 1.57E−05 | 5.32E−03 |
| Kmo | 6 | 0.551 | 0.658 | −0.233 | 1.62E−05 | 5.44E−03 |
| Sla | 6 | 0.313 | 0.26 | 0.413 | 1.62E−05 | 5.46E−03 |
| Asph | 6 | 0.198 | 0.331 | −0.557 | 1.84E−05 | 6.08E−03 |
| Nrros | 6 | 0.195 | 0.336 | −0.536 | 2.11E−05 | 6.87E−03 |
| Crem | 6 | 0.22 | 0.389 | −0.664 | 2.16E−05 | 6.98E−03 |
| Ppp1cc | 6 | 0.477 | 0.612 | −0.237 | 2.26E−05 | 7.24E−03 |
| Tmem59 | 6 | 0.65 | 0.585 | 0.239 | 2.55E−05 | 8.06E−03 |
| Csnk2b | 6 | 0.405 | 0.37 | 0.220 | 3.23E−05 | 9.87E−03 |
| Hhex | 6 | 0.358 | 0.301 | 0.200 | 3.50E−05 | 1.06E−02 |
| Cnn2 | 6 | 0.44 | 0.381 | 0.284 | 3.56E−05 | 1.08E−02 |
| Cdc42se2 | 6 | 0.314 | 0.466 | −0.423 | 3.63E−05 | 1.10E−02 |
| Hmgb2 | 6 | 0.356 | 0.375 | 0.286 | 3.67E−05 | 1.11E−02 |
| Slc15a3 | 6 | 0.133 | 0.246 | −0.605 | 4.98E−05 | 1.44E−02 |
| Eif3d | 6 | 0.336 | 0.284 | 0.318 | 5.06E−05 | 1.46E−02 |
| Rnf149 | 6 | 0.365 | 0.493 | −0.419 | 5.17E−05 | 1.49E−02 |
| Clec12a | 6 | 0.8 | 0.783 | 0.164 | 5.72E−05 | 1.63E−02 |
| Aldoa | 6 | 0.539 | 0.502 | 0.274 | 6.04E−05 | 1.70E−02 |
| Cxxc5 | 6 | 0.373 | 0.508 | −0.279 | 6.11E−05 | 1.72E−02 |
| Mndal | 6 | 0.36 | 0.467 | −0.366 | 6.25E−05 | 1.75E−02 |
| Polr3gl | 6 | 0.333 | 0.481 | −0.372 | 7.10E−05 | 1.95E−02 |
| Pafah1b1 | 6 | 0.282 | 0.424 | −0.322 | 7.38E−05 | 2.02E−02 |
| Rsrp1 | 6 | 0.508 | 0.638 | −0.235 | 8.19E−05 | 2.20E−02 |
| Cdv3 | 6 | 0.292 | 0.428 | −0.426 | 8.32E−05 | 2.23E−02 |
| Ppp1r2 | 6 | 0.146 | 0.27 | −0.566 | 8.41E−05 | 2.25E−02 |
| Fcrla | 6 | 0.617 | 0.599 | 0.212 | 8.57E−05 | 2.28E−02 |
| Plekho2 | 6 | 0.096 | 0.221 | −0.682 | 9.24E−05 | 2.43E−02 |
| Mdh2 | 6 | 0.56 | 0.508 | 0.212 | 9.63E−05 | 2.52E−02 |
| Tmem108 | 6 | 0.166 | 0.304 | −0.730 | 1.01E−04 | 2.62E−02 |
| Stat1 | 6 | 0.203 | 0.331 | −0.414 | 1.02E−04 | 2.64E−02 |
| Snx18 | 6 | 0.529 | 0.676 | −0.354 | 1.05E−04 | 2.73E−02 |
| Sh3glb1 | 6 | 0.371 | 0.541 | −0.388 | 1.07E−04 | 2.75E−02 |
| Blnk | 6 | 0.61 | 0.715 | −0.297 | 1.08E−04 | 2.79E−02 |
| Rbm5 | 6 | 0.203 | 0.332 | −0.496 | 1.09E−04 | 2.80E−02 |
| Jakmip1 | 6 | 0.187 | 0.339 | −0.505 | 1.12E−04 | 2.87E−02 |
| Ppp2ca | 6 | 0.461 | 0.573 | −0.283 | 1.16E−04 | 2.96E−02 |
| Kpna4 | 6 | 0.239 | 0.184 | 0.355 | 1.23E−04 | 3.12E−02 |
| Pkib | 6 | 0.566 | 0.692 | −0.235 | 1.28E−04 | 3.22E−02 |
| Ndufa13 | 6 | 0.738 | 0.703 | 0.169 | 1.30E−04 | 3.26E−02 |
| Iqgap1 | 6 | 0.439 | 0.57 | −0.347 | 1.35E−04 | 3.36E−02 |
| Itpkb | 6 | 0.146 | 0.266 | −0.621 | 1.35E−04 | 3.37E−02 |
| Cyth4 | 6 | 0.545 | 0.674 | −0.190 | 1.45E−04 | 3.56E−02 |
| Wnk1 | 6 | 0.334 | 0.504 | −0.543 | 1.55E−04 | 3.76E−02 |
| Npm3 | 6 | 0.403 | 0.366 | 0.213 | 1.55E−04 | 3.77E−02 |
| Mef2a | 6 | 0.331 | 0.394 | −0.217 | 1.55E−04 | 3.77E−02 |
| Fam117a | 6 | 0.136 | 0.209 | −0.376 | 1.62E−04 | 3.90E−02 |
| Ubl3 | 6 | 0.412 | 0.561 | −0.334 | 1.68E−04 | 4.03E−02 |
| Gsn | 6 | 0.622 | 0.587 | 0.184 | 1.73E−04 | 4.14E−02 |
| Bri3 | 6 | 0.41 | 0.558 | −0.335 | 1.83E−04 | 4.33E−02 |
| Chrac1 | 6 | 0.198 | 0.32 | −0.486 | 2.15E−04 | 4.96E−02 |
| Ighg1 | 7 | 0.407 | 0 | 5.050 | 3.59E−18 | 7.66E−15 |
| Gm2a | 7 | 0.642 | 0.398 | 1.626 | 5.92E−12 | 6.60E−09 |
| Actb | 7 | 0.992 | 0.971 | 0.962 | 3.97E−11 | 4.06E−08 |
| Il17rb | 7 | 0.293 | 0.019 | 3.210 | 2.95E−10 | 2.66E−07 |
| Smco4 | 7 | 0.659 | 0.32 | 1.788 | 5.54E−10 | 4.77E−07 |
| Gata3 | 7 | 0.618 | 0.388 | 1.226 | 2.27E−09 | 1.76E−06 |
| Igkv1-135 | 7 | 0.016 | 0.252 | −2.815 | 2.39E−09 | 1.84E−06 |
| Igha | 7 | 0.992 | 0.99 | 0.455 | 3.88E−09 | 2.84E−06 |
| Gimap4 | 7 | 0.439 | 0.738 | −0.828 | 7.29E−09 | 5.13E−06 |
| Jun | 7 | 0.569 | 0.194 | 1.121 | 1.42E−08 | 9.54E−06 |
| Dusp5 | 7 | 0.341 | 0.68 | −0.905 | 1.95E−08 | 1.28E−05 |
| Fgl2 | 7 | 0.455 | 0.194 | 1.771 | 3.97E−08 | 2.44E−05 |
| Mcpt1 | 7 | 0.195 | 0 | 4.373 | 4.26E−08 | 2.60E−05 |
| Vps37b | 7 | 0.39 | 0.728 | −0.964 | 5.97E−08 | 3.54E−05 |
| Ddit4 | 7 | 0.366 | 0.107 | 2.414 | 7.06E−08 | 4.12E−05 |
| Ubald2 | 7 | 0.325 | 0.65 | −0.844 | 9.80E−08 | 5.56E−05 |
| Lgmn | 7 | 0.35 | 0.078 | 2.276 | 1.05E−07 | 5.97E−05 |
| 1700061F12Rik | 7 | 0.244 | 0.019 | 3.537 | 2.06E−07 | 1.11E−04 |
| Ifngr1 | 7 | 0.496 | 0.757 | −0.885 | 6.29E−07 | 3.06E−04 |
| Cst7 | 7 | 0.463 | 0.136 | 1.346 | 6.44E−07 | 3.13E−04 |
| Il4 | 7 | 0.317 | 0.068 | 2.197 | 9.09E−07 | 4.26E−04 |
| Zeb2 | 7 | 0.203 | 0.01 | 3.094 | 2.54E−06 | 1.06E−03 |
| Igkc | 7 | 1 | 1 | 0.756 | 3.05E−06 | 1.23E−03 |
| Ccl5 | 7 | 0.114 | 0.34 | −1.521 | 7.96E−06 | 2.91E−03 |
| Ltb | 7 | 0.463 | 0.777 | −0.502 | 8.51E−06 | 3.08E−03 |
| Pik3r1 | 7 | 0.374 | 0.67 | −0.770 | 1.32E−05 | 4.56E−03 |
| Foxp3 | 7 | 0.138 | 0.369 | −1.433 | 3.47E−05 | 1.05E−02 |
| Il21r | 7 | 0.13 | 0.379 | −0.811 | 4.26E−05 | 1.26E−02 |
| Foxp1 | 7 | 0.431 | 0.223 | 1.129 | 4.28E−05 | 1.26E−02 |
| Dusp1 | 7 | 0.87 | 0.709 | 0.631 | 4.64E−05 | 1.36E−02 |
| Tmsb4x | 7 | 1 | 1 | 0.377 | 6.83E−05 | 1.89E−02 |
| Got1 | 7 | 0.171 | 0.379 | −1.199 | 8.56E−05 | 2.28E−02 |
| Pxdc1 | 7 | 0.154 | 0.01 | 3.051 | 9.85E−05 | 2.57E−02 |
| Fas1 | 7 | 0.033 | 0.184 | −1.603 | 1.07E−04 | 2.75E−02 |
| Cd27 | 7 | 0.48 | 0.563 | −0.759 | 1.17E−04 | 2.97E−02 |
| Txk | 7 | 0.065 | 0.252 | −1.234 | 1.52E−04 | 3.70E−02 |
| Tmsb10 | 7 | 0.976 | 1 | −0.179 | 1.56E−04 | 3.78E−02 |
| Gzma | 7 | 0.317 | 0.117 | 1.230 | 1.57E−04 | 3.79E−02 |
| Pim1 | 7 | 0.52 | 0.728 | −0.660 | 1.98E−04 | 4.63E−02 |
| Icos | 7 | 0.488 | 0.738 | −0.538 | 2.09E−04 | 4.84E−02 |
| Ikzf2 | 8 | 0.099 | 0.26 | −1.020 | 1.37E−07 | 7.61E−05 |
| Ubald2 | 8 | 0.464 | 0.644 | −0.714 | 2.09E−07 | 1.12E−04 |
| Ppp1r15a | 8 | 0.304 | 0.575 | −0.642 | 4.53E−07 | 2.28E−04 |
| Litaf | 8 | 0.326 | 0.582 | −0.663 | 2.58E−06 | 1.07E−03 |
| Gimap7 | 8 | 0.519 | 0.247 | 0.937 | 1.08E−05 | 3.81E−03 |
| Orai1 | 8 | 0.127 | 0.349 | −1.213 | 1.29E−05 | 4.46E−03 |
| Zc3h12a | 8 | 0.077 | 0.288 | −1.472 | 1.31E−05 | 4.52E−03 |
| Dusp2 | 8 | 0.613 | 0.404 | 0.686 | 2.44E−05 | 7.76E−03 |
| Prr7 | 8 | 0.066 | 0.24 | −1.385 | 2.58E−05 | 8.13E−03 |
| Btg1 | 8 | 0.912 | 0.973 | −0.402 | 3.21E−05 | 9.84E−03 |
| Irf7 | 8 | 0.138 | 0.322 | −1.262 | 3.96E−05 | 1.18E−02 |
| Zfp36 | 8 | 0.376 | 0.144 | 1.132 | 5.81E−05 | 1.65E−02 |
| Tob2 | 8 | 0.11 | 0.253 | −1.231 | 5.81E−05 | 1.65E−02 |
| Rgs1 | 8 | 0.691 | 0.486 | 0.864 | 9.21E−05 | 2.42E−02 |
| VgIl4 | 8 | 0.331 | 0.555 | −0.657 | 1.07E−04 | 2.75E−02 |
| Sh2d1a | 8 | 0.431 | 0.247 | 0.847 | 1.22E−04 | 3.10E−02 |
| Igkc | 8 | 1 | 1 | 0.307 | 1.27E−04 | 3.20E−02 |
| Jun | 8 | 0.343 | 0.151 | 1.129 | 1.32E−04 | 3.32E−02 |
| Vps37b | 8 | 0.702 | 0.836 | −0.490 | 1.33E−04 | 3.33E−02 |
| Ctla2a | 8 | 0.464 | 0.425 | 0.531 | 1.35E−04 | 3.37E−02 |
| Nr4a2 | 8 | 0.265 | 0.493 | −0.758 | 1.36E−04 | 3.38E−02 |
| Pim1 | 8 | 0.63 | 0.815 | −0.376 | 1.53E−04 | 3.73E−02 |
| Sptssa | 8 | 0.42 | 0.199 | 0.904 | 1.71E−04 | 4.09E−02 |
| Ifrd1 | 8 | 0.232 | 0.425 | −0.686 | 1.80E−04 | 4.27E−02 |
| 4930523C07Rik | 8 | 0.215 | 0.425 | −0.724 | 1.83E−04 | 4.33E−02 |
| H2-D1 | 8 | 1 | 0.993 | −0.212 | 2.09E−04 | 4.84E−02 |
| Al504432 | 9 | 0.057 | 0.636 | −2.126 | 4.42E−05 | 1.30E−02 |

TABLE 4
A-K. Top 50 genes in each topics revealed by topic modeling on T cells, B cells,
DCs, myeloid cells and stromal cells in PP and LP regions, related to FIG. 3.
A. T cells LP

| topic_1 | topic_2 | topic_3 | topic_4 | topic_5 | topic_6 | topic_7 | topic_8 |
|---|---|---|---|---|---|---|---|
| mt-Co1 | Eef1a1 | Igfbp4 | Gimap5 | 1700061F12Rik | H3f3b | Malat1 | Actb |
| mt-Atp6 | Gm8730 | Dapl1 | Gabarap | Il13 | Ier2 | Zfp369 | Pdcd4 |
| Gmfg | Gm10260 | Ccr7 | Rbck1 | Scin | Junb | Gtdc1 | Cbx3 |
| Myl12b | Eef2 | Pdlim4 | Cops4 | Lgmn | Tnfaip3 | Dusp23 | Abhd2 |
| Sfxn2 | Gm10709 | Lef1 | mt-Nd1 | Hlf | Pim1 | 5330439K02Rik | Ccdc38 |
| Socs3 | Fau | Bcl2 | Bcl7c | Smco4 | Ppp1r15a | 5430421N21Rik | Tnfrsf19 |
| Cd53 | Naca | Sell | Iqgap1 | Npnt | Dusp5 | Ltk | Ncoa3 |
| Acp5 | Eif3k | Ifngr2 | Ralbp1 | Il17rb | Crem | Mdm4 | Il17f |
| Gm12840 | Eif3f | Gm8369 | Ier3ip1 | Deptor | Atf4 | Snapc3 | Ptpn22 |
| Mbp | Pabpc1 | Satb1 | Dock10 | Gata3 | Ramp3 | Gm28424 | Trav3-1 |
| Rasal3 | Btf3 | Gm9889 | 0610030E20Rik | Gm2a | Nfkbia | Gm16894 | Sv2a |
| Atxn7l1 | Tmsb10 | Smc6 | Ptbp1 | Il6 | Jund | Stk3 | Fxyd4 |
| mt-Nd2 | Wdr89 | Hotairm1 | Emp1 | Il17a | Nr4a1 | Klhl4 | Lta |
| Gltscr2 | Cct4 | Khdrbs3 | Scand1 | Ltb4r1 | Tgif1 | Serinc2 | Ankrd23 |
| Ets1 | Kif1b | Actn1 | Zfp672 | Fgl2 | Pnrc1 | Herc2 | Alkbh5 |
| Son | Bola2 | Ms4a4b | Lrmp | Areg | Ier5 | Apc | Bend5 |
| Kcnn4 | Pelp1 | Rapgef4 | Usp9x | Fbxl21 | Eif1 | Pgap3 | Ptp4a2 |
| Uqcrfs1 | Eef1b2 | Amigo2 | Gm10814 | AA467197 | Nr4a3 | P4htm | Nthl1 |
| Cd3d | Dkc1 | Atp1b1 | mt-Nd3 | Il1rl1 | Zfp36 | Ptpre | Caprin2 |
| Lrp12 | Mrps33 | Eml5 | Emd | Mel | Sub1 | Tnrc6a | Zfp763 |
| Casp8 | Gm10073 | Sh3bp5 | Sipa1 | Gm5544 | Calm2 | Map1b | Tctn1 |
| Gypc | Gm2000 | Trav17 | Dnajc14 | Tmem159 | Dnajb6 | Antxr2 | Gga2 |
| Ap5b1 | Nhp2l1 | Btg1 | Trappc4 | Rasgrp4 | Ifrd1 | Trbv19 | Armcx6 |
| Tes | Gnb2l1 | F2rl1 | Akap13 | 1700012B07Rik | Zc3h12a | Uhrf2 | Sostdc1 |
| Trim26 | Eif6 | Gm11808 | Desi1 | 1700113H08Rik | Traf1 | Aagab | Gm43625 |
| Arid5a | Gm9493 | St8sia6 | Srebf2 | St6galnac5 | Srsf2 | Cr1l | Fcer2a |
| Abcb9 | Rexo2 | S1pr1 | Gngt2 | Il4 | Tgif2 | Ei24 | Carm1 |
| Stk17b | Gm6133 | Klhdc2 | Uqcrb | Chdh | Kras | Gm17518 | Nrg4 |
| Snrpd3 | Atp1a1 | Foxp1 | Sppl3 | Slco2b1 | Klf2 | Lix1l | Tlr7 |
| Hmha1 | Klrb1a | Npc2 | Tmem167 | Ccr9 | Rnf125 | Dmrta1 | Tmem121 |
| Rragd | Sepw1 | Saraf | Ssbp2 | Epas1 | Bud31 | Uchl4 | Slc39a1 |
| Wnk1 | Emc6 | 2610020C07Rik | Sec61g | Grp | Il2rg | Sgtb | Gm42809 |
| Arhgap15 | Prorsd1 | Myo19 | Manbal | Lztfl1 | Cd69 | Nfam1 | Fitm2 |
| Siah1a | Ndufa6 | Ranbp6 | Fkbp3 | Gm10369 | Csrnp1 | Ackr3 | Dqx1 |
| Tcf25 | Nhp2 | Cmah | Fis1 | Kif19a | Apoe | Arhgef9 | Clec10a |
| Wdr33 | Gm9843 | Treml2 | Kdm2b | Tenm4 | Cep85 | Ighv1-64 | Ankrd60 |
| Cbx4 | Yeats4 | Drc1 | Fip1l1 | Serpinf1 | Coq10b | Trav9-2 | Gm14154 |
| RP23-428N8.3 | Gm26545 | Prrg1 | H2afv | Gnb2 | Gpr132 | Gm3716 | Upf1 |
| Gna1 | Ctdsp1 | Usp28 | H2-Q10 | Ubox5 | Cd209b | Evx1 | Marf1 |
| Clec2l | Eef1d | Atp1b3 | Hdgfrp2 | Plcl1 | Pcna-ps2 | Plxnb2 | Actr2 |
| Cd247 | Rbfa | Rab3ip | Nsmce1 | Rab31 | Fus | Ascl3 | Mapkbp1 |
| Gltp | Pfdn5 | Trib2 | Fastk | Ffar2 | Cap2 | Gm8973 | 1110035H17Rik |
| Rock1 | Eif5a | Rpusd2 | Ndufb7 | Slx1b | Ifnar1 | Hhex | Eid2b |
| Eif3i | Atp5e | Il4ra | Ndufs4 | Asb2 | Nfkbie | Armt1 | Kalrn |
| Ctss | Ndufa10 | Il6ra | Prdx5 | Zfp85 | Ube2b | Ccdc86 | P3h4 |
| Ccm2 | Cycs | Whsc1l1 | Tbl1xr1 | Tmsb4x | Dus2 | Hbegf | Notch2 |
| Zmat2 | Ppp1r14b | Ggt1 | Sf3b5 | Hdc | Birc3 | Stkld1 | Mcpt1 |
| Fxyd5 | Tomm7 | Prdx6 | Tomm34 | Pxdc1 | Hnrnph1 | Zswim3 | Gskip |
| Bnip3l | Snrpf | Pygm | Bend4 | Heatr1 | Osgep | Edaradd | Dpysl2 |
| Dnajc25 | Cox7a2l | Itga6 | Gng7 | Lgals7 | Igkv5-37 | C030034L19Rik | Lsm14b |

| topic_9 | topic_10 | topic_11 | topic_12 | topic_13 | topic_14 | topic_15 | topic_16 |
|---|---|---|---|---|---|---|---|
| Tnfrsf4 | Ube2c | Fos | Trpm1 | Crip1 | B2m | Cel5 | Iglc2 |
| Foxp3 | Actg1 | Hspa1a | Prss2 | Ccr2 | H2-K1 | Nkg7 | Igkc |
| Tnfrsf9 | Lgals1 | Rgs1 | Gm26825 | Emp3 | Ubb | Klra7 | Jchain |
| Cd83 | Ccna2 | Jun | Zfp266 | Zfat | H2-D1 | Gzmm | Igha |
| Ikzf2 | Tuba1b | Klf6 | Kifap3 | Klf3 | Gimap4 | 4930486L24Rik | Mzb1 |
| Izumo1r | Hmgb2 | Dnajb1 | Mareks | Map4 | Psme1 | Cd200r2 | Iglc1 |
| Mmd | Tubb5 | Hist1h3a | Igkv4-50 | Plekha6 | Gm28707 | Gzma | Hsp90b1 |
| Itgb8 | Fam64a | Hsp90aa1 | Acp2 | Glipr2 | Itpripl2 | Xcl1 | Pdia4 |
| Penk | Mybl2 | Gm29666 | Ptpn23 | Myl10 | Polr1b | Cd8a | Txndc5 |
| Ifi27l2a | Cfl1 | Ampd2 | Rpgrip1 | Atrnl1 | Isgl5 | Cd7 | Mef2c |
| Tnfrsf18 | Birc5 | Btg2 | Gm17275 | Zfp318 | Siae | Klrd1 | Creld2 |
| Ctla4 | Pttg1 | Tiparp | Ccdc51 | Ptpn18 | Igtp | Cel3 | Derl3 |
| Rgs10 | Stmn1 | Pygl | Ppp1r12b | Recql5 | Cul5 | Ccl4 | Cacna1e |
| Zfp36l1 | Cenpk | Cops6 | Klhl35 | Dennd1a | Ighv1-80 | Ppp2r2c | Arx |
| Nrip1 | Slc30a2 | Ppp1r3b | Sumf2 | Gm26740 | Gm14306 | Zmat4 | Ftx |
| Gpm6b | Cenpf | Gle1 | Sh3pxd2a | Gm37696 | Ifi35 | Gzmk | Gm43121 |
| S100a11 | Actr3 | Efna4 | Dcaf13 | Mboat4 | Tbc1d31 | Pdzk1 | Fkbp2 |
| Capg | Gapdh | Litaf | Arl11 | Tnnt3 | Tifab | Klrk1 | Sdf2l1 |
| Il10 | Rad54l | Anks3 | Cnr2 | Ppp1r14a | Travl6n | Slc16a11 | Edem1 |
| Lag3 | Eme1 | Dtx4 | Abcb6 | Ift74 | AW112010 | Car2 | Cd79a |
| Tiam1 | Nek2 | Tab2 | C230037L18Rik | Klrb1b | Psmb10 | Dact2 | Iglv1 |
| Matk | Ect2 | Ahcyl1 | Gm26910 | Odf2l | Shisa5 | Adrb1 | Fkbp11 |
| Wisp1 | Cntnap2 | 1110001J03Rik | Oasl1 | Arl6ip5 | Xrcc2 | Ablim2 | Manf |
| Gas2l3 | Bcam | Dusp1 | Bin3 | Il23r | Cyp26b1 | Slc9a3r2 | Slc7a11 |
| Fgf16 | H2afx | Fam103a1 | Gtf2ird2 | Ppplrl2a | Rarres2 | Cited4 | Rab25 |
| Hist1h4c | 2810417H13Rik | Nabp1 | Dgcr8 | Zfp40 | Ighv14-2 | Klra5 | Mfsd2a |
| Ccl20 | Ccnb2 | A630001G21Rik | Anapc2 | Thy1 | Armc2 | Serpinb6b | Rhd |
| Slc7a10 | Rad51ap1 | Fam189b | Gcfc2 | Elf4 | Dapk1 | Lmo1 | Pla2g5 |
| Fam46a | Pfn1 | Osgin1 | Caleb | Lsp1 | Zfp964 | Tlr6 | Ccdc18 |
| Ptprcap | Esco2 | Qpct | Gstt1 | 2010320M18Rik | Tapbp | Pdgfb | Bhlhal5 |
| H1f0 | Neil3 | Myo7a | Pard3b | Adcy7 | A730015C16Rik | RP24-330M21.1 | Bcat1 |
| Tbc1d4 | Kif4 | Susd4 | Thnsl1 | Csl | Cntd1 | Rasgef1c | Arfgef3 |
| Ankrd55 | Arhgap19 | Al847159 | Gm14321 | Ulk3 | Zbp1 | Emilin2 | Gm867 |
| Bcar3 | Hist1h2ap | Evx1os | Eng | Mcm9 | Krit1 | A930011G23Rik | Fabp3-psl |
| Gm3488 | Top2a | Adamts14 | Gps1 | Cpne4 | Cacng8 | Gm11491 | Mettl21b |
| Nefm | Cks1b | Stac3 | Ddr1 | Spaca6 | Egfl8 | Ccdc14 | Mcemp1 |
| Alox8 | Cotl1 | Rhoq | Rasl11a | Abcc10 | Gm8444 | 1810006J02Rik | Arhgef40 |
| Celf5 | Cdkn3 | Nox1 | Gm17089 | Enpp2 | Tinf2 | Trgv2 | Piwil2 |
| Slc26a11 | Rbm3 | Wdr19 | Eme2 | Slc27a1 | Mbnl3 | Tcrg-C2 | 2010007H06Rik |
| Camk2n1 | Myl12a | Sema7a | Gm12454 | Lancl3 | Gbp4 | Tlr12 | Plekhh3 |
| Pdcd1 | 270094K13Rik | Anks6 | Gm43740 | Ighv1-15 | Smpd1 | Wdr95 | Spata32 |
| Zan | Kif18a | Gm17056 | Mtus2 | Ccne2 | Rnf13 | Itln1 | Cyb561 |
| Gm3739 | Plk1 | Lamtor2 | Mdfi | Fbxl13 | Psmb9 | Ifng | Apoh |
| Gm3667 | Lockd | Tgfbr1 | Gm43691 | Gm43153 | Cyp4f18 | 3110082l17Rik | Arsg |
| Tnfsf8 | Hist1h2ag | Gm10031 | Zcchc24 | Arg2 | Mvk | 1700021A07Rik | Mgat3 |
| Maf | C330027C09Rik | Dapk3 | Gm28731 | Slco4a1 | Mccc1 | Nr4a2 | Endou |
| Egln3 | Trip13 | Dnaja4 | Lrrc27 | Rab34 | Stat1 | Htra1 | Gm20517 |
| Gm3636 | BC030867 | 5033417F24Rik | Zfp937 | Gpr25 | Got1 | Gm27194 | Pank1 |
| Spock2 | Sgol2a | Ehbp1 | Coasy | Gm20750 | Gbp2b | Cd8b1 | Pbx1 |
| Myo1h | 4930524J08Rik | Serpinb1b | Slc9b2 | Nos3 | Myof | Klrc1 | Phka1 |

B. B cells LP

| topic_1 | topic_2 | topic_3 | topic_4 | topic_5 | topic_6 | topic_7 | topic_8 |
|---|---|---|---|---|---|---|---|
| Igkc | Igha | Mzb1 | Cd74 | Jchain | Fos | Malat1 | Crip1 |
| Igkv6-23 | Rec1l4 | Hsp90b1 | Tmsb4x | Ighg2c | mt-Nd1 | Gm42418 | Psma7 |
| Ighv6-7 | Igkv3-8 | Sec61b | H2-Aa | Iglv1 | Jun | Ccdc88a | Srsf6 |
| Igkv8-27 | Stab1 | Ppib | H2-Eb1 | Ly6c2 | mt-Atp6 | Gm26917 | Dnaja2 |
| Igkv12-41 | Tmem147os | Sub1 | H2-Ab1 | Ly6c1 | Jund | Txndc5 | Nop56 |
| Pomgnt2 | Gm14327 | Manf | Fau | Edem2 | Fosb | Rbm39 | Trp53il1 |
| Slc1a1 | 4930451G09Rik | Calr | H2-DMb2 | B2m | Nfkbia | Gm42726 | Eif4g2 |
| Ighv1-30 | Ighv1-19-1 | Pycard | Ebf1 | Insl6 | H3f3b | Zfp292 | Hypk |
| AY074887 | 9530052E02Rik | Hspa5 | H2-Ob | Procr | mt-Co3 | Kmt2e | Cnbp |
| Igkv9-124 | Gm14399 | Pdia6 | Fcer2a | Dmtn | mt-Cytb | Rrbp1 | Ahsa1 |
| Cbs | Gm26596 | Ighg2b | Ighd | Selm | Dusp1 | Krr1 | Cox7b |
| Igkv17-127 | Platr17 | Sec61g | Cd79b | Iqcd | Dusp5 | 4930523C07Rik | Wbp11 |
| Gm15104 | Iqca | Pdia4 | Ms4a1 | Iglv3 | Cacna1s | Creb3l2 | Psmc5 |
| Syce1 | Myo3b | Dnajb11 | 4930597A21Rik | Ighv1-15 | Ier2 | Slc48a1 | Ndufa5 |
| Gm26527 | Igkv9-123 | H13 | Pax5 | Gm15284 | mt-Co2 | Zdhhc14 | Mrpl21 |
| Abca17 | Ttyh1 | Sdf2l1 | Cxcr5 | 1700047l17Rik2 | mt-Nd4 | Ankhd1 | Ociad1 |
| Nsg2 | Gnrh1 | Tnfrsf17 | Cd72 | Sdc1 | Ftl1 | Upf2 | Pfdn6 |
| Gm17529 | Fabp1 | Krtcap2 | Sapcd1 | Copz2 | Mt1 | Nfkbil1 | Arpc2 |
| Spin4 | Krt7 | Ddost | H2-DMa | Gm20878 | Ier5 | Zmynd11 | Mrpl40 |
| Col5a3 | Zfp174 | Ssr2 | Eef1a1 | Ryr1 | Ppp1r15a | Gm26719 | Nsun2 |
| A830009L08Rik | Cdh24 | Ssr3 | Cd22 | Gpnmb | Cxci10 | Cyld | Ubtf |
| Cps1 | Gm26515 | Reep5 | Ly86 | Pycr1 | mt-Co1 | A630072M18Rik | Cmtm7 |
| Clec4a4 | Rhobtb3 | Lman1 | H2-Oa | Cst6 | Rilpl2 | Rbm26 | Rrp1 |
| Col4a2 | Gm15445 | Rpn1 | Cox7a2l | Igfn1 | Pnrc1 | Nek7 | Morf4l1 |
| Prox2os | Mdfi | Emp3 | Eif3f | Cdnf | Ubald2 | Cspp1 | Ndufb5 |
| Pdc | Cfap53 | Pdia3 | 1-Mar | Bhlha15 | Lmna | Map3k10 | Tomm22 |
| 1700019B03Rik | Tal2 | Srp9 | Eef2 | Ighv1-23 | Hes1 | Zfp51 | Ndufa7 |
| Ighv1-61 | Zp3 | Fkbp11 | Rac2 | Vps37d | Herpud1 | Mvp | Pcnp |
| Lcat | Nr3c2 | Spcs2 | Ccr7 | Akap6 | Egr1 | 2510039018Rik | Gars |
| Igkv8-16 | Rdh1 | Spcs1 | Fcmr | Igkv1-110 | Ly6d | Map3k1 | Basp1 |
| Ripk4 | Tmem202 | Ift20 | Spib | Extl1 | Serp1 | Ppt1 | Pafah1b3 |
| D930048G16Rik | Gm43616 | Ostc | Dusp2 | Ckmt1 | Cd69 | Vwa1 | Mrpl11 |
| RP23-138J20.8 | Frzb | Sec11c | Rapgef4 | Ero1lb | mt-Nd3 | Rabac1 | Srrm1 |
| Sec31b | Kcna2 | Cope | Cotl1 | Fam71b | Mt2 | Alyref2 | Csnk2b |
| Gm20632 | Gm37027 | Derl3 | Gm9843 | Ighv1-20 | Rhob | Gm26586 | Abcf1 |
| Etv2 | Padi3 | Cd52 | Siglecg | Gm20478 | Xbp1 | Ggnbp2 | Caprin1 |
| Gm17034 | Aqp7 | Rpn2 | Coro1a | Gm5134 | mt-Nd2 | Slc16a6 | Ddx21 |
| Wisp2 | Gm14453 | Slc35b1 | Pold4 | Gm29375 | Mel1 | Nacc2 | Serbp1 |
| RP24-159C12.10 | Dusp27 | Ssr4 | Bin1 | Dlg2 | Rgcc | Gigyf2 | Mrps33 |
| Tbx15 | Slc6a12 | Tmbim6 | Bank1 | Gpr31b | Zfp36l1 | Clec2d | Mrfap1 |
| Mlana | Scn2a1 | Lman2 | Cd37 | Kcnmb4os2 | Ode1 | Arhgap22 | H2afy |
| Synm | Hoxb1 | Creld2 | Gm43603 | Ighv10-3 | Clic4 | Ankrd40 | Eif1b |
| Gm26663 | 1700001L19Rik | Ccr10 | Rgs13 | Atp6v0e2 | Txnip | Cped1 | Cnot2 |
| Gm43479 | Pabpn1l | Atp5g1 | Zfp318 | Rnf123 | Atf4 | Fam214a | Oaz1 |
| Gm12735 | Dynlrb2 | Srprb | Scd1 | F420014N23Rik | Fam46c | St6ga1nac4 | Pfn1 |
| Gm19902 | Mb21d2 | Os9 | Blk | Btd | Chac1 | Ddx46 | Dnajc8 |
| Sema6c | Gm19345 | Erp44 | Cnn2 | Gdap2 | 5031425E22Rik | Fam117a | Arf1 |
| A530013C23Rik | Tmem253 | Scpep1 | 4930426D05Rik | Nron | Klf2 | Stk25 | Mrpl54 |
| Nav3 | Gm42749 | Kdelr1 | Fth1 | Gm26725 | Hist1h2bc | RP24-172H24.1 | Chchd10 |
| Cacna1f | Sstr2 | Srp72 | Napsa | Gm12474 | Ddit3 | Tnfrsf13b | Eif3a |

| topic_9 | topic_10 | topic_11 | topic_12 | topic_13 | topic_14 | topic_15 | topic_16 |
|---|---|---|---|---|---|---|---|
| 2810417H13Rik | Iglc1 | Xist | Mcpt1 | Ighg1 | Iglc2 | Slpi | Ighm |
| Birc5 | Iglc3 | Peli1 | Lyz2 | lgkv3-4 | Igkv9-119 | Actb | Phactr1 |
| Stmn1 | Pcsk5 | Mef2c | Mcpt2 | Pon3 | Igkvl4-111 | Igkv3-7 | Igkv16-104 |
| Hmgb2 | Gm43409 | Tcf4 | Tyrobp | Cldn4 | Iglv2 | Ighv1-18 | Ccdc155 |
| Ptma | Mc1r | Arl2bp | Ighg3 | Kcnh2 | Iglc4 | Stt3b | Nr6a1os |
| Tubb5 | 1700017D01Rik | Akap13 | Ccl4 | Nat8 | Gm11444 | Tsc22d3 | Gm648 |
| Ube2c | Lrrc46 | Alkbh5 | Ighv2-3 | Hey2 | Trav13-1 | Sdc4 | Smoc2 |
| Tuba1b | Gm28198 | Fli1 | Gzma | Gm14210 | Gm28513 | Gprl71 | Fhad1 |
| Nusap1 | Sst | Ctbp1 | Cpa3 | Kcnq4 | Cacna2d1 | lghv1-21 | Gm26840 |
| Hist1h2ap | Igll1 | Tmem57 | Psap | Gm29395 | Mettl21c | Cited2 | 1700061G19Rik |
| H2afz | Tnr | Vgll4 | Ccl5 | Hist1h1t | Trbv14 | Gm37511 | Stkld1 |
| Cenpf | Tenm3 | Hmgn3 | Plac8 | Nmur1 | BC049352 | Kdelr2 | Asb18 |
| Rrm2 | Slc38a5 | Dennd4a | Trbc2 | Pip5kl1 | Prom1 | Ywhaq | Gm26646 |
| Cdkn3 | Gm20468 | Igkv12-46 | Maf | Atp6v1g3 | Necab3 | Pole2 | Gm43569 |
| Ccnb1 | Angptl7 | Insig1 | Cma1 | D930007J09Rik | lgkv1-99 | Tram1 | Gm15819 |
| Ube2s | Gm10719 | Tgoln1 | Il1b | Fbxo24 | Gm17151 | Vpreb3 | Fmo2 |
| Ccna2 | Pcdh19 | Psd4 | Ifitm1 | Plxnb1 | Gm43734 | Rbm47 | Lrrc74b |
| Cenpe | Hpca | Marf1 | Id2 | Gm28551 | Gm44175 | Pitpnc1 | Fer1l6 |
| Ran | Plvap | Ddx3x | 1700061F12Rik | Trim66 | Pappa | Lgals1 | Ncs1 |
| Uhrf1 | Gm26681 | Hmg20b | AW112010 | Drp2 | 2610528Jl1Rik | Atp6v1g1 | Pdilt |
| Cep55 | Gnao1 | Skil | Cxcl2 | Gm16494 | 4930481A15Rik | Naa38 | Casq1 |
| Top2a | Gja5 | Bcl21l1 | Apoe | Gdap1l1 | Eci3 | Dap | Ly6k |
| Kif22 | Klkb1 | Cnppd1 | Ifitm2 | RP23-53O7.2 | Muc2 | Slc39a1 | Gm26780 |
| Cdc20 | Gm19434 | C87436 | Mpeg1 | Boll | D630029K05Rik | Entpd7 | Gm14137 |
| Plk1 | Fgf16 | Slc25a36 | Trbc1 | Tsx | Gfra2 | Zbtb20 | Cpq |
| Cdc45 | Ccdc96 | Mdm4 | Cd7 | Gm16982 | Gm15356 | Tox | Tmbim7 |
| H2afx | D7Ertd443e | Wnk1 | Cox6a2 | Apol6 | Rlbp1 | Gm19585 | lgkv4-57 |
| Cdca3 | D030040B21Rik | Ccm2 | Mcpt9 | Gm42957 | Gm10516 | Upf1 | Igkv2-112 |
| Mcm5 | Tmcc2 | Prkcd | Furin | Irx5 | Smkr-ps | Timmdc1 | Slc7a7 |
| Cdca8 | Al661453 | Elf1 | Kdm6b | lgkv4-63 | Mfsd7c | Eaf2 | Gp2 |
| Pbk | Gm44037 | Tomm5 | Il4 | Iqch | Adamts12 | Pycr2 | 9430069l07Rik |
| Ccnb2 | Gm26550 | Rbms2 | Alox5ap | Slc27a2 | Gm37240 | Khdc1a | Csn3 |
| Mybl2 | Tmem45a2 | Phf3 | Igkv12-40 | Apol8 | Art3 | Xlr4b | Zfp316 |
| Nuf2 | K230010J24Rik | 1300002E11Rik | C1qc | Gm26883 | Nlrp1b | Emc10 | Mpp7 |
| Hmmr | lghv3-4 | Ubxn2a | Fcer1a | Shank2 | Clca3b | Fkbp8 | Gm43218 |
| Cit | Cdhr1 | Nktr | Lgals3 | Ighv1-43 | Rab33a | Yrdc | Igkv4-86 |
| Pttg1 | Ighv1-53 | Slc38a2 | Cd63 | Igkv1-88 | Pcdhb19 | Kcnn4 | Prg2 |
| Spc24 | Npm2 | Bhlhe41 | Siglech | Mx1 | Gm42669 | Arf4 | Rab3b |
| Hmgn2 | 5730460C07Rik | Birc2 | Actn2 | 1700023F06Rik | Defa-rs1 | Dnajb9 | 3222401L13Rik |
| Esco2 | Cebpe | Coa5 | Itm2b | Abcc3 | Pglyrp4 | Per3 | Myo5c |
| Cdk1 | Vtn | Safb2 | Igkv19-93 | Klhl40 | Flrt1 | Tmem50a | Ccdc3 |
| Ckap2l | Gm12666 | Ddx50 | Nkg7 | Igkv8-18 | Tpbg | Fn3krp | D630003M21Rik |
| Chaf1a | Rem2 | Rbm25 | Cxcr6 | 1700010K24Rik | Gm15895 | Uqcrq | Tom1l1 |
| Ybx1 | Gm26765 | Zfp787 | Adamdec1 | Samd3 | 3110070M22Rik | Pcca | Gm6034 |
| Neil3 | Gm15978 | Ube2h | Gata3 | A930006L05Rik | Cadps | Eif5b | Tspan32os |
| Smc4 | lghv1-55 | Alkbh1 | Mcpt4 | Klhl31 | Necab1 | Luc7l2 | Stox2 |
| Asf1b | RP23-366014.5 | Raf1 | Zfp36l2 | Gm4787 | 5430402013Rik | Igkv12-38 | C1ra |
| Cks2 | BC043934 | Ppcdc | Lair1 | Gm12333 | Mptx2 | Tagln2 | Sox12 |
| Cks1b | Gm26707 | Bcl7c | Ccl3 | 1700088E04Rik | AY761184 | Tmem173 | C2 |
| Smc2 | Gm19684 | Dennd5b | Emb | Gzmk | Tc2n | Ccdc117 | 2610528A11Rik |

C. DCs LP

| topic_1 | topic_2 | topic_3 | topic_4 | topic_5 | topic_6 |
|---|---|---|---|---|---|
| Siglech | Ctla2b | Lyz2 | Stmn1 | Cd74 | Ccl17 |
| Cox6a2 | Xcr1 | Il22ra2 | 2810417H13Rik | H2-Ab1 | Malat1 |
| Klk1 | Ppt1 | Siglece | Ptma | H2-Aa | Ccl22 |
| Cd8b1 | Cst3 | Plet1 | Ube2c | H2-Eb1 | Ftl1 |
| D13Ertd608e | Naaa | S100a11 | Hmgb2 | Ifitm3 | Ccr7 |
| Sh3bgr | Tlr3 | Cxcl14 | Tuba1b | Wfdc17 | Cxcl1 |
| Pacsin1 | A530099J19Rik | Mmp12 | Birc5 | Clec4b1 | Flrt3 |
| Ccl4 | Hebp2 | Gp2 | Tubb5 | Cd209a | Fscn1 |
| Sell | Actg1 | Ppplrl4a | Rrm2 | H2-DMb1 | Lad1 |
| Ly6c2 | Plpp1 | B4galt6 | Ccnb2 | Ifitm2 | Cacnb3 |
| Lefty1 | Ptgis | Vasp | Nusap1 | H2-DMb2 | Bcl2a1d |
| Smim5 | Eef1b2 | Atox1 | Ran | Cd209b | Cdkn1a |
| Bst2 | Cxx1a | Atp5e | Ccna2 | Socs3 | Ankrd33b |
| Tyrobp | 2310031A07Rik | Fpr2 | Cdca3 | Srgn | Ccl5 |
| Ly6cl | Atpif1 | Sec61b | Ranbp1 | Smpdl3a | Tmem123 |
| Ighm | Vwf | Ifitm6 | Ppia | H2-D1 | Cxcl2 |
| Cdh5 | Ucp2 | Sh3bgrl3 | H2afx | Gadd45g | Cxcl16 |
| Dnajc7 | Txndc15 | Msrb1 | Cks1b | Hfe | Bcl2a1b |
| Ctsb | Cxx1b | Ccr1 | Tk1 | Trf | Arl5c |
| Dntt | Rundc3a | Tnni2 | Top2a | Hcls1 | Gadd45b |
| Gm14964 | Acvrl1 | Arpc1b | Anp32b | Gsn | Apol7c |
| Prkca | Psmb9 | Mcemp1 | Cep55 | Cd53 | Basp1 |
| Lair1 | Aif1 | S100a6 | Erh | Ms4a4c | Bcl2a1a |
| Atp2a1 | Agpat3 | Cyp4f18 | Cdkn3 | Atp6ap2 | Il4i1 |
| Ccr9 | Cd207 | Trem1 | Ybx1 | App | Etv3 |
| Mctp2 | Fam149a | Tctex1d2 | Ckap2l | Bcap31 | Cd83 |
| Timp2 | Psme1 | Igsf6 | Cenpe | Hexb | Serpinb9 |
| Iglc3 | 1700009J07Rik | Lyz1 | Ccnb1 | Ms4a6d | Nudtl7 |
| Klk1b27 | Rab32 | Il1rn | Alyref | Cysltr1 | Traf1 |
| Pir | Plbd1 | Serf2 | Hmgb1 | Bicd2 | Cxcl10 |
| Nrxn2 | Gm11837 | Spi1 | Ska1 | Gm5150 | Zmynd15 |
| mt-Atp6 | Ttc39a | Fam189a2 | Tmpo | Itm2b | Tspan3 |
| Tbxa2r | Rgs10 | Tgfbi | Cdca8 | Dab2 | Samsn1 |
| Grn | Tmsb4x | Adamdec1 | Prc1 | Csf2ra | Mt2 |
| Dap | Cd52 | Bcas1 | H2afz | Susd3 | Csrp1 |
| Blnk | Rab7b | Cox4i1 | Dut | Ebp | Lrrc32 |
| Atp1b1 | Cd36 | Atp5l | Cenph | Milr1 | Clic4 |
| Clec12a | Qpct | Stra6l | Cdca7 | Man2b1 | Il12b |
| Gm5547 | Lpar6 | Clec4a4 | Npm1 | Itgam | Marcksl1 |
| Gm12253 | P3h2 | Il23a | Mcm6 | Slc44a5 | Map4k4 |
| Il12a | Proser2 | Pald1 | Prdx1 | Ptger3 | Mxd1 |
| Scimp | Gm28411 | Sirpa | Melk | Myl12a | Cd40 |
| Rpgrip1 | Col25a1 | Myeov2 | Snrpd1 | Ppp1r13l | Tnnt2 |
| Pgls | Sult1a1 | Nedd8 | Pttg1 | Dmkn | Cdkn2b |
| Paqr5 | Snx22 | Atp6v1f | Cdk1 | Mgl2 | Rgs1 |
| Fcrla | Cyp26bl | Tarm1 | Nuf2 | Ctsc | Lilr4b |
| Gm19705 | Pmf1 | Tsga10ip | Snrpb | Aph1c | Fam49a |
| Irf7 | Kremen2 | Clec5a | Hnrnpa3 | Ticam1 | Tnfaip2 |
| Hpse | Gm6377 | Cox6c | Bub1 | AF251705 | Eno3 |
| Havcr1 | Commd8 | Calml4 | Pfn1 | Gm43936 | Il1a |

| topic_7 | topic_8 | topic_9 | topic_10 | topic_11 | topic_12 |
|---|---|---|---|---|---|
| Junb | Actb | Fth1 | Klf2 | Igkc | S100a4 |
| Btg2 | Gm2a | Tpt1 | Fos | Igha | Mcpt1 |
| Nr4a1 | Cbx3 | mt-Co2 | Jun | Jchain | Mcpt2 |
| Pim1 | Notch2 | Xist | Dusp1 | Iglc2 | Nccrp1 |
| Atf4 | Skil | Fau | Neat1 | Iglc1 | Gm15848 |
| Zfand5 | Kdelr2 | Gnb2l1 | Ubb | Txndc5 | Prr29 |
| Fosb | Slc39a1 | Btg1 | Hspa8 | Gm43291 | C1qb |
| Ppp3ca | Ncoa3 | mt-Nd1 | Jund | Cd79a | Ltb |
| Ddx5 | Lrrc58 | Eef2 | Alox5ap | Trp53inpl | Gpx4 |
| Ptbp3 | Upf1 | Gm10709 | Klf6 | Iglv1 | Mif |
| Zfos1 | Rbm15 | Eef1a1 | Zfp36l2 | Mzb1 | Aif1l |
| Jarid2 | Alkbh5 | mt-Nd3 | Tspo | Slpi | Krt8 |
| Picalm | Kdm6b | Gm8730 | Dnajb1 | Derl3 | Gata3 |
| Cmip | Insig1 | Wnk1 | Fxyd5 | Ssr4 | Lum |
| Dusp5 | Otub1 | mt-Co1 | Cd48 | Edem1 | Mdk |
| Pip4k2a | Fn3krp | Vps37b | Ccnd3 | Pou2afl | Ndufa1 |
| Mat2a | D17H6S53E | Crem | Pmaip1 | Hsp90b1 | Cfp |
| Tob2 | Mcmbp | Bcl2l11 | Fcer1g | Fkbp11 | Crybb2 |
| Ndel1 | Wtap | Tgfb1 | Lsp1 | Sec11c | Cib1 |
| Mel1 | Marf1 | Srrm2 | Arhgdib | Vpreb3 | Klrg1 |
| Gna13 | Abhd2 | Ptpn18 | Bin2 | Sdc1 | S100a9 |
| Ythdc1 | Yme1l1 | Stat3 | Zfp36 | lgkv3-7 | Kcnk1 |
| Ubc | Soat1 | Ssh2 | Cyp7b1 | Ccr10 | G0s2 |
| Pde4b | Cdk6 | Gm42418 | Itgb7 | Iglv3 | S100a3 |
| Plekho2 | Hsf2 | Ubl3 | Tsc22d3 | Lax1 | Platr3 |
| Cbl | Ric1 | Gcnt2 | Zbtb32 | Tnfrsf17 | PISD |
| Sf3b1 | 4933434E20Rik | Eif3h | Atp5c1 | Pdia4 | Sri |
| Ctage5 | Paip1 | Klf13 | Plp2 | a | Cma2 |
| Eif4g3 | Lyrm4 | mt-Co3 | Sin3b | Xbp1 | C1qa |
| Tiparp | Pdcd4 | Map3kl | Eif4a1 | Eaf2 | Cited4 |
| Stk38 | Rrn3 | Slc3a2 | Itm2c | Spcs1 | Serpinb1a |
| Dock10 | B4galtl | Mrfap1 | Ier5 | St6gal1 | Lysmd2 |
| Jmjd1c | Timmdc1 | Oaz1 | Enol | Cd55 | Prdx5 |
| Herpud1 | St13 | Nsa2 | Cd209e | Edem2 | Foxs1 |
| Ankrd11 | Bmp2k | St8sia4 | Ms4a6c | Rein | Id2 |
| Brd2 | Dfna5 | Ywhaz | Sema4a | Lman1 | Lamp1 |
| Clk1 | Kmt2d | Gpcpd1 | Egr1 | Endou | Rac2 |
| Csrnp1 | Dnase1l3 | Ago2 | Fgd2 | Hid1 | Nrgn |
| Gm26917 | 2310035C23Rik | Smc6 | Rhob | Gm26826 | Bcam |
| Pten | Klhl9 | Itpkb | Capns1 | Mgat3 | Clic3 |
| Ddx21 | Tmf1 | Son | Fcgrt | 9230117E06Rik | Trbc1 |
| Gnaq | Gm15800 | Pmepa1 | Hnrnpk | Gm15634 | Cxcr6 |
| St3gal4 | Acot2 | Ahr | Pepd | Actg2 | Ly9 |
| Wsb1 | Zscan26 | Luc7l2 | Ifi30 | Gm17096 | Gem |
| Alcam | Ppm1h | Fam43a | Lmo1 | Gm9796 | Tnfsf11 |
| Kdm7a | Pan3 | Kras | Ccl9 | Txndc11 | Vsig2 |
| Ifrd1 | Myef2 | Ifnar1 | Capzb | Chchd10 | Capg |
| Gm6133 | Lrrc8a | Tsix | Pdcd10 | Il5ra | Plekhs1 |
| 5031425E22Rik | Slc2a12 | Dph3 | Coro1a | Bhlha15 | S100a10 |
| Tm9sf3 | Pfas | Gng5 | Gltp | Znrf3 | Limd2 |

D. Myeloid cells LP

| topic_1 | topic_2 | topic_3 | topic_4 | topic_5 | topic_6 | topic_7 | topic_8 |
|---|---|---|---|---|---|---|---|
| Cpa3 | Lyz2 | Cd74 | Malat1 | S100a9 | 2810417H13Rik | Mcpt1 | Ccl2 |
| Cma1 | S100a4 | H2-Aa | Mmp13 | S100a8 | Stmn1 | Mcpt2 | Hspa1a |
| Mcpt4 | Ifitm3 | H2-Abl | Apoe | G0s2 | H2afz | Igkc | Gm26522 |
| Tpsb2 | Ccl9 | H2-Eb1 | Dnase1l3 | Retnlg | Tubb5 | Igha | Dnajb6 |
| Fcer1a | Ms4a4c | Retn1a | Ccl8 | Il1b | Ptma | Jchain | Ccl7 |
| Hs3st1 | F13a1 | H2-DMb1 | Lilra5 | Cxcl2 | Birc5 | Mcpt9 | Tmem261 |
| Gata2 | Chil3 | Mgl2 | Clec1b | Gm5483 | Ube2c | Ifitm1 | Nr4a2 |
| Cited4 | Thbs1 | H2-DMb2 | Ccl4 | Wfdc21 | Mcpt8 | Cd63 | Gm6133 |
| Cyp11a1 | Id3 | Tmsb4x | Amica1 | Lrg1 | Rrm2 | Cma2 | Mrpl30 |
| Tph1 | F10 | H2-DMa | Acp5 | Asprv1 | Hist1h2ap | Apol9b | Jtb |
| Furin | Serpinb10 | Aif1 | C6 | Lcn2 | Ppia | Iglc2 | Ppp3ca |
| Rab27b | Ifi27l2a | Tmem176b | Apol7c | Irg1 | Spc24 | Ctla2a | Jarid2 |
| Slc45a3 | Gpx1 | Cd83 | Gas6 | Ccrl2 | Hmgb2 | Itgae | Serinc3 |
| Cel1 | Crip1 | Lyz1 | Pla2g7 | Il1f9 | Hmgb1 | Apol9a | Idh2 |
| Il13 | Al839979 | Tmem176a | Stab1 | S100a11 | Nusap1 | Mzb1 | Zfos1 |
| Il1rl1 | Ace | Cd209a | Gas1 | Msrb1 | Ccnb2 | Otop1 | Poldip3 |
| Itga2b | Cst3 | Slamf8 | Pla2g2d | Mrgpra2b | Top2a | Cpne5 | Panx1 |
| Cited2 | Ly6c2 | Mmp12 | Ftl1 | Dusp1 | Tuba1b | Prnp | Fam96a |
| Fam110c | Smpdl3a | Axl | Mertk | Stfa2l1 | Spc25 | Gzma | Ppp1r9b |
| Creb3l1 | Ifitm6 | Cxcl16 | Sash1 | Hcar2 | Cdca3 | Pga5 | Pigt |
| Rgs13 | Fn1 | Gm15056 | Apol10b | Pglyrp1 | Cdc20 | Iglv1 | Bckdha |
| Tpsab1 | Lgals3 | Pltp | Adamdec1 | Marcksl1 | Ccna2 | Kcnn4 | Fh1 |
| Cyp26a1 | Plac8 | Pdgfb | Gm19434 | Nfkbia | Cks1b | Htra1 | Rtf1 |
| Serpinb1a | Fau | Psap | Ubc | Fth1 | H2afx | Asb17os | Snhg6 |
| Slc18a2 | Nupr1 | Mrc1 | S1c15a3 | Trem1 | H3f3b | Creg1 | Grsf1 |
| Gmpr | Ccr2 | Mpeg1 | Ccl3 | BC100530 | Cdca8 | Ldlrad4 | Rsl24d1 |
| Rprm | Arpc2 | Mt1 | C1qa | Igfbp3 | H1f0 | Thsd4 | Zc3h7a |
| Ero1l | Galnt9 | Slamf9 | Atf3 | Csf3r | Aurkb | Adgrg1 | Eif4e3 |
| Il4 | Apoc1 | Ctss | Cebpa | Clec4e | Tpx2 | Colec11 | Dtx3l |
| Cd200r3 | Ly6c1 | Ccl12 | Ppbp | HP | H2afv | Sec14l2 | Dram2 |
| Glul | Tppp3 | Gatm | Cd81 | Cebpb | Ran | Iglc3 | Dock2 |
| Kit | Tnfsf13os | Rgl1 | Cxcl1 | Slc16a3 | Kif22 | BCl2l15 | Sertad2 |
| Lat | Vim | Sdc3 | Klf6 | Steap4 | Hmgn2 | Ssr4 | Tnfrsf13b |
| Alox5 | Stxbp6 | Itgb5 | Ctsl | Mfap4 | Cks2 | Speg | Srsf6 |
| Gchfr | Shfm1 | Ppfia4 | Ms4a7 | Ly6g | Ube2s | Zcchc18 | Mbnl1 |
| mt-Atp6 | Dmkn | Ctsh | Vps37b | Clec4d | Alyref | Fkbp11 | Tmem192 |
| Lat2 | Wfdc17 | Cfp | Pgf | Il23a | Asf1b | Urocl | Tcea1 |
| Prss34 | Pitpna | Ctsc | Csf1r | Mrgpra2a | Pbk | Ulk4 | Cat |
| Poln | Cd52 | Plxdc2 | Pmepa1 | Osm | Ccnb1 | Ldhc | Slk |
| Klk8 | B2m | Ifi30 | Rgs1 | Chil1 | Fbxo5 | Iglc1 | Prdx2 |
| 4932438H23Rik | Cd300lg | Il22ra2 | Sat1 | Cxcr2 | Ranbp1 | Gm43291 | Gm26825 |
| Slc6a13 | Clec4a3 | B4galt6 | Fcna | Mxd1 | Ncapd2 | Fermt2 | Vps41 |
| Avil | Lmo1 | Plekho1 | Rhob | Cyp4f18 | Cenpa | Ppa1 | Smim20 |
| Socs2 | Arpc1b | Batf3 | Gpx3 | Gadd45b | Ska1 | Ndrg2 | Pabpn1 |
| Smco4 | Ahnak | Rogdi | Tsku | Plpp3 | Racgap1 | Mro | Psma5 |
| Ier3 | Actr3 | Dab2 | AF251705 | Cdk2ap2 | Cit | Cux2 | Phf20 |
| Lxn | Emp1 | Amdhd1 | Ltc4s | Ltb | Rad51 | Adamts1 | Vav1 |
| Gpr171 | Hopx | Tbc1d9 | C1qc | Mmp8 | Anp32b | Slc6a9 | Os9 |
| Adk | Glud1 | Sdc4 | Irf8 | Dhrs9 | Mcm6 | Mbtps1 | Comt |
| Gata1 | Ms4a8a | Bvht | Hal | Ctsd | Ncapg | 6030468B19Rik | Mfsd1 |

E. ILCs LP

| topic_1 | topic_2 | topic_3 | topic_4 | topic_5 | topic_6 | topic_7 |
|---|---|---|---|---|---|---|
| Rnase4 | Calca | Il22 | Id2 | Malat1 | Ccl5 | Dusp5 |
| Gda | Hs3st1 | Cd83 | Tmem59 | Shisa5 | Gzma | Bcl2a1d |
| Rcbtb2 | Areg | Odc1 | Dbp | Aim2 | Klrd1 | Serpinb9 |
| Tcrg-C1 | Il13 | Cx3cl1 | Itm2b | Amz2 | Ms4a4b | Rgs1 |
| Upp1 | Il4 | Cxcr5 | Med23 | Tnfaip8l2 | Klre1 | Klrk1 |
| Ly6g5b | Ccl1 | Cd74 | Bcl2 | Il18r1 | Klra3 | Gimap4 |
| Cd163l1 | Hes1 | Nrp1 | Tmx2 | Pdcd4 | Sh2d1a | Tmsb10 |
| Lrrn2 | Il17rb | Il17f | Paqr7 | Gm26917 | Klra9 | Pim1 |
| Ucp3 | Lgals7 | Gpx1 | Cep57 | Itch | Klra7 | Cd69 |
| Gapdh | Homer2 | Dil1 | Ttc14 | Aldh3a2 | Eomes | Irf7 |
| Ppp1r14c | Il5 | Igkc | More1 | Cd96 | Samd3 | Pfn1 |
| Pcp4 | Gata3 | Tox2 | Uqcc1 | Icos | Serpinb9b | Serpinb6b |
| Aprt | Deptor | H2-Ab1 | Stat5b | Pnisr | Gzmb | Bhlhe40 |
| Abi3bp | Ptpn13 | Gem | Prkd3 | Dhcr24 | Tyrobp | Mxd1 |
| Serf2 | Ly6a | Prss2 | Sec62 | Crebzf | Ccl4 | Hk2 |
| Ucp2 | Hba-a1 | Foxq1 | Vhl | Cebpz | Sema4a | Fasl |
| Cxcr6 | Kcnn4 | Lmna | Cept1 | Sap30bp | Cd2 | Hcst |
| Dmrta1 | Ccr4 | Cacna1g | Smg6 | Gab3 | Ccr5 | Gimap3 |
| Rbpms2 | Rxrg | Foxs1 | Tnfaip1 | 1600014C10Rik | Trdc | Nabp1 |
| Cox6bl | Sub1 | Parvb | Rnfl14 | Fus | Nkg7 | Serpinb1a |
| Ptprz1 | 1700061F12Rik | Adra1b | Ncln | Fam65b | Phactr3 | Tnfaip3 |
| Sla | Cntnap2 | Fcgrt | Mcfd2 | Stk19 | Il21r | Slc16a6 |
| Uqcr11 | AA467197 | Ramp1 | Phc3 | Rsrp1 | Itgam | Selplg |
| Capg | Ptgir | H2-Eb1 | Bod1 | Spns1 | Irf8 | Litaf |
| Efhd1 | Il10 | Fam110a | Snapin | Cab39l | Lgals1 | Smap2 |
| Atp5j2 | Nfkb1 | Bst2 | Tmem11 | Rbm39 | Gzmk | Lgals3 |
| Gm13180 | Lmo4 | Hoxa7 | Smn1 | Hbp1 | Actb | Gadd45b |
| 4933431E20Rik | Pparg | Hebp1 | Gimap1 | Luc7l2 | Gramd3 | Vps37b |
| 2010107E04Rik | Plaur | H2-Aa | Pitpnb | Slc7a6os | Anxa2 | Pim3 |
| Minos1 | Il9r | Xist | Champ1 | Spryd3 | Klra5 | Car2 |
| Myeov2 | Serpine1 | Platr3 | Phf20 | Tmem127 | Tm6sf1 | Srsf12 |
| Gabbr2 | Seel | Sox4 | Chd8 | Them6 | Gm15518 | Abi3 |
| Cfl1 | Bmp7 | Edf1 | Pwwp2a | Bnip3l | Cd247 | Ikzf2 |
| Cryba4 | Neb | Ostf1 | Fra10ac1 | Ypel3 | Ifitm10 | Lck |
| Sit1 | Sox8 | Rrad | Wdr82 | Ankrd11 | Cd52 | Pltp |
| Gm26652 | Lpcat2 | Stab1 | Zmynd8 | N4bp2l1 | Dusp2 | Ggct |
| Gm9949 | Samsn1 | Fermt2 | Jtb | Itpr2 | Gpx8 | Ncr1 |
| Tns4 | Alox5 | Jchain | Cdc26 | Rpusd4 | BC094916 | Neurl3 |
| Ybx1 | Gpr65 | Slc16a2 | Cnot1 | Gm16223 | Atp1b1 | Vasp |
| Nop10 | Abhd17c | Ninj1 | Tor2a | Gps2 | Klra4 | Phlda1 |
| Gimap9 | Gm20186 | Cd4 | Zfp866 | Oard1 | Fcrl6 | Ctsw |
| Rinl | Gm973 | Cd81 | 1110034G24Rik | Kdelc2 | Lat2 | Nfil3 |
| Tomm7 | Epas1 | Cd40 | Nudt9 | Cdc23 | Sell | Clcf1 |
| Tmem64 | Ccr8 | Krt25 | Trim12c | Sbds | Ifng | RP23-218E6.4 |
| Mrps33 | D430036J16Rik | Adra2a | Otud6b | Mgea5 | Tmsb15a | Ezr |
| Clic1 | Cd6 | 5930412G12Rik | Cited4 | Tk2 | Klra1 | Sept6 |
| Pfdn1 | Stxbp6 | Cldn10 | Nfx1 | Usp7 | Kcnj8 | Pabpc1 |
| P2rx7 | 9230102004Rik | Piwil4 | Baz1b | Sirt7 | Myb | Kctd12 |
| Tnp2 | Furin | Pacsin1 | Zbtb43 | Ing1 | Ptprc | Ifngr1 |
| Sema4b | Klf5 | Marcksl1 | Mphosph8 | Hipk1 | Ugcg | Cd160 |

| topic_8 | topic_9 | topic_10 |
|---|---|---|
| Fos | Zfp740 | Il1b |
| Junb | H1f0 | Ncoa7 |
| Dusp1 | Usb1 | S100a8 |
| Ier2 | B930036N10Rik | Tubb5 |
| Zfp36 | Ppp1r10 | Tuba1b |
| Jun | Puf60 | Rasl11a |
| Klf2 | Adprm | Hmgb2 |
| Btg2 | Bod1l | Stmn1 |
| Nr4a1 | Ppp2r1b | Ltb |
| Fosb | Dhx30 | S100a9 |
| Klf4 | Pdcl | Ttyh2 |
| Ppp1r15a | Slc35c1 | 2810417H13Rik |
| Pnrc1 | Jade2 | Cks1b |
| Egr1 | Elk3 | Hmgn2 |
| Gm26532 | Psmb10 | Rgcc |
| Klf6 | Tnrc6b | Ube2c |
| Iglc1 | Osbpl2 | Hspa1a |
| H2-T23 | Rhob | Chad |
| Jund | Thap3 | Rorc |
| Irf1 | Mad2l2 | Igf1r |
| Rnf2 | Trim26 | Cd7 |
| Ier5 | Sec16a | Sdc4 |
| Fuca2 | Golga7 | Cks2 |
| Sgf29 | Pgm2l1 | Ddc |
| Arid4a | Uxt | Birc5 |
| Gm8797 | Yif1a | Top2a |
| Ap3b1 | Ccdc47 | Hist1h2ap |
| Nt5c3 | Tpp1 | Apoe |
| Ip6k1 | Wdr45b | Cbx3 |
| Uvrag | Mcpt1 | Pbk |
| Amn1 | Pde4dip | H2afx |
| Mir142hg | Zfand2a | Pxdc1 |
| Trrap | Rbm5 | M1ap |
| Snhg12 | Unc93b1 | Smc2 |
| Pias2 | Lonp1 | Asf1b |
| Vps41 | Ppp3r1 | Spc24 |
| Per1 | Ppp2ca | Retnlg |
| Ppp5c | Neil1 | Ccdc34 |
| Sgta | Xrcc6 | Cenpa |
| Slain2 | Brix1 | Tipin |
| Tbc1d10a | Rbm15b | G0s2 |
| Tiparp | Prmt1 | Rrm2 |
| Get4 | Mdh1 | Smc4 |
| Xpa | Arcn1 | Cdc20 |
| Pomgnt1 | Dynlt3 | S1pr3 |
| Cmip | Polr2g | Tifa |
| Vamp2 | Map4 | Siva1 |
| Stim1 | Zfp830 | Ccna2 |
| Sp2 | Eif2b4 | Fbxo5 |
| H2-Q4 | Pip5k1c | Dhrs3 |

F. Stromal cells PP

| topic_1 | topic_2 | topic_3 | topic_4 |
|---|---|---|---|
| B2m | Glycam1 | Dcn | Ccl21a |
| Cd81 | Tm4sf1 | Mt1 | Cxcl13 |
| Gm26917 | Fabp4 | Col3a1 | Clu |
| Malat1 | Ly6c1 | Gsn | Ccl19 |
| Igfbp7 | Lrg1 | Adamdec1 | Acta2 |
| Lmna | Ctla2a | Serping1 | Mfge8 |
| Eif3f | Tmem252 | Rarres2 | Apoe |
| Arpc1b | Pecam1 | C3 | Tagln |
| Ndufa4 | Egfl7 | Col1a1 | Cxcl1 |
| Gnas | Plvap | Rgs5 | Cilp |
| Atp5l | Flt1 | Col1a2 | Cel2 |
| Atp6v1g1 | Scgb3a1 | Cel11 | Il33 |
| Srsf5 | Apold1 | Plac8 | Cxcl12 |
| Ppib | 8430408G22Rik | Clec3b | Actg2 |
| Erh | Cldn5 | Gem | Serpina3n |
| Ndufb11 | Sox17 | Bmp4 | Ccl7 |
| Rab2a | Ace | Col6a2 | Bst1 |
| Pfdn5 | Cdh5 | Cebpb | Serpina1a |
| Srsf3 | Emcn | Ifi27l2a | Fmod |
| Pfn1 | Esam | Mt2 | Grem1 |
| Cox6b1 | Ptprb | Phlda1 | Serpina1b |
| Uqcr10 | Aqp1 | Ms4a4d | Slc36a2 |
| Sept4 | Cd93 | Fbln1 | Cnn1 |
| Gm10260 | Esm1 | Col6a1 | Myh11 |
| Socs3 | Rasip1 | Mgp | Art2b |
| Cope | Kdr | Tcf21 | Actc1 |
| Fkbp8 | Cd36 | Lrp1 | Al838599 |
| Ranbp1 | Icam2 | Crispld2 | Serpina1c |
| Tagln2 | Myct1 | Cxcl14 | Cr2 |
| Ube2n | Cav1 | Lpl | Gxylt2 |
| Fos | Mgll | Spon1 | Crym |
| H2-Q7 | Edn1 | Olfml3 | Dclk1 |
| Gm13889 | Caim1 | Spon2 | Serpina1d |
| Fus | Arhgap31 | Aspn | Myl9 |
| Slc25a5 | Ushbp1 | Bgn | Parm1 |
| Map1lc3b | Actg1 | Fth1 | Gm16685 |
| Zfos1 | Ackr1 | Col14a1 | Postn |
| Pnrc1 | Gpihbp1 | Sdc2 | Chrdl1 |
| Ube2i | C1qtnf9 | Pid1 | Colq |
| Atp5o | Klf2 | Col6a3 | Csn2 |
| Ubald2 | Mecom | Cd302 | Prss12 |
| Pkig | Cdkn1a | Mmp2 | H2-M2 |
| Chmp4b | Ptprr | Col5a2 | Trf |
| Zcrb1 | Rnd1 | Pdgfra | Sostdc1 |
| Mel1 | Gngt2 | Cygb | Dsc3 |
| Maf1 | Tek | Gm12840 | Ctgf |
| Spop | Tspan13 | Mmp10 | Thbs4 |
| mt-Atp8 | Sema3g | Mxra8 | Pcdh15 |
| Dctn3 | Adgrg1 | Ctsk | Rtn4r |
| Ubb | St3gal6 | Cpxm1 | A230065H16Rik |

G. T cells PP

| topic_1 | topic_2 | topic_3 | topic_4 | topic_5 | topic_6 | topic_7 | topic_8 |
|---|---|---|---|---|---|---|---|
| Igfbp4 | Tmsb10 | Arhgdib | Malat1 | Klf2 | Pdcd1 | Ptma | Trbc1 |
| Gm8730 | Pfn1 | Pik3ip1 | Xist | Btg1 | Il4 | Ppia | Sepp1 |
| Eef1a1 | Sepw1 | Gmfg | mt-Co1 | Dusp10 | Gal | Tuba1b | Cd53 |
| Eef2 | Ccnd2 | Coro1a | Fyb | Vps37b | Nav2 | Eif5a | Smc6 |
| Gm10709 | Cycs | Lsp1 | mt-Co3 | H3f3b | Tox2 | Ran | Stk38 |
| Gm10260 | Psmg4 | Cdkn2d | mt-Cytb | Ccr7 | Cebpa | Slc25a5 | Tcf7 |
| Naca | Rrs1 | Sell | mt-Atp6 | Junb | Sostdc1 | Mif | Hsdl1 |
| Fau | Nolc1 | Grap | mt-Nd5 | Pnrc1 | Angptl2 | Pa2g4 | Cdc42se1 |
| Eif3f | Bel2 | Scp2 | Inpp4b | Ifngr2 | 5430421N21Rik | Nme1 | Atp1b3 |
| Pdlim4 | Gar1 | Tuba1a | Atrx | Gm8369 | Gfra4 | Ranbp1 | Skap1 |
| Tpt1 | Map2k2 | Tmem59 | Zfp941 | Rgs2 | Tbc1d4 | Serbp1 | Atp1b1 |
| Btf3 | Zfp593 | Al467606 | Stx16 | Gpcpd1 | Rgs10 | Atp5g1 | Add3 |
| Eif3k | Rpf2 | Emc9 | Zmynd12 | Frat2 | Gpm6b | Fbl | Mycbp2 |
| Crip1 | Atn1.1 | Def6 | Rb1cc1 | Dusp5 | Cxcr5 | Hmgn1 | Tmem234 |
| Pabpc1 | Trmt10c | Rnf114 | Map1a | P2ry10 | Bcl2a1b | Ddx39 | Tspan14 |
| Gm10269 | Srm | Samd9l | Taco1 | Tnfaip3 | Cd200 | Eno1 | 4930523C07Rik |
| Eif3e | Tmem248 | Rsrp1 | Ufsp1 | Zfp281 | Smco4 | Ybx1 | Prkd2 |
| Eef1b2 | Rsl1d1 | Ostc | Gm28306 | Bambi | Izumo1r | Eif4a1 | Egln2 |
| Nacc2 | Med28 | Mpnd | Vwa1 | Cd28 | Ascl2 | Uqcrq | Lamtor2 |
| Nsa2 | Ywhaq | B3galt4 | Prrc2c | Il4ra | Vdr | Apex1 | Rp9 |
| Gm13546 | Gng5 | Sgk3 | Zkscan8 | Btg2 | Il21 | Anp32b | Itk |
| Eif3h | Impdh2 | Triap1 | 4933427l04Rik | Cxcr4 | Adam33 | Tcp1 | Ms4a6b |
| Cox4i1 | Mlx | 2410002F23Rik | Gm27241 | Ssh2 | Ucp2 | Banf1 | Ikzf1 |
| Gm26888 | Mettl1 | Ric8 | Bcl11b | Ptp4a1 | Gna14 | Nop10 | Card19 |
| 2010107E04Rik | Rasgrp2 | Rgs19 | Ssbp2 | Ets2 | Cdk2ap2 | Prdx1 | Itm2a |
| Eif3i | Pwp2 | Glud1 | Cul9 | S1pr1 | Ptprcap | Psma7 | G3bp2 |
| Tsga10ip | Armc1 | BC029722 | Rbm39 | Tagap | Chn1 | Hnrnpa3 | Il17ra |
| Psmb1 | Synj2bp | Ppp1ca | Gm16152 | Gm20186 | Lgals1 | Uqcc2 | Raf1 |
| Atp5g2 | Atad3a | Psap | Zmym5 | Arid5a | lgkv3-2 | Ppa1 | Zfp292 |
| Pfdn5 | Abce1 | Rbm10 | Ankrd12 | Csrnp1 | Sh2d1a | Snrpe | Usp53 |
| Hnrnpa1 | Psmd3 | Smarcd2 | Vps13a | Gpr18 | Rac2 | Mcm2 | Faah |
| Uqcrh | Nop16 | Leprotl1 | Fus | Dnajb9 | Maf | Hprt | Ndrg3 |
| Slpi | Psme3 | Mettl9 | Tmx4 | Gpr183 | Krt17 | Erh | Actr10 |
| Use1 | Ddx18 | Sat1 | Tmem40 | Socs3 | Gm2762 | Ndufa12 | Klf7 |
| Eif4b | Myd88 | Bin1 | Ddrgk1 | Nabp1 | Fgf2 | C1qbp | Hgs |
| Snrpf | Trmt1 | Vps4a | Cecr6 | Maff | Gm9885 | Cox7b | Pde12 |
| Gltscr2 | Aars | Ankrd13a | Oxt | Mirt1 | Sh3gl3 | Lsm7 | Gimap6 |
| Uqcrfs1 | Msl2 | Phyh | Erich4 | Trim13 | Vamp8 | Snrpd2 | Rasgrp1 |
| Myeov2 | Gps1 | Ubald1 | Gm44226 | Ifnar1 | Serpinf1 | Atp5j | Mir142hg |
| 2700060E02Rik | 2810004N23Rik | Limd2 | Gm26559 | Prkca | Il9r | Atp5j2 | 2410016O06Rik |
| Vipr1 | Fam50a | Med25 | Zbtb20 | Fosl2 | Ctsb | Tubb5 | Slc35b3 |
| Gnb2l1 | Lsm12 | Pafah1b2 | Gm28044 | Rnf125 | Ubl5 | Prmt1 | Trac |
| Gm17541 | Dnaja3 | Gtf3a | RP23-272F2.2 | Sgms1 | Fam20a | Cox6a1 | Ncoa2 |
| Tomm7 | Ccnd3 | Map4k2 | 2810030D12Rik | Il2rg | Fst | Ncl | Cd3d |
| Srms | Polr2h | Ppa2 | Arhgef17 | Bcl2l11 | Bcl2a1d | Stmn1 | Toporsos |
| Emg1 | Utp18 | Tbc1d10c | Adam6b | Gramd3 | Actg2 | Srsf3 | Upf2 |
| Sec61g | Nfkbib | Gm15675 | 2610528J11Rik | Sgip1 | Smox | Grwd1 | Mlec |
| Tbc1d16 | Nop2 | Daxx | Plch2 | Tubb2a | Cpeb1 | Lyar | Tpp2 |
| mt-Co2 | Pisd | Slc28a2 | Hist1h2ad | Gypc | Dhx34 | Gpn1 | 1600020E01Rik |
| Gm6096 | Thap7 | Exosc7 | Pcdhgb2 | Prdx6 | Kirrel3 | Mrpl23 | Rnf167 |

| topic_9 | topic_10 | topic_11 | topic_12 | topic_13 | topic_14 | topic_15 | topic_16 |
|---|---|---|---|---|---|---|---|
| Hspa1a | Wdr89 | Ctla4 | Dusp2 | Actb | Ccl5 | Cd8b1 | Ifi27l2a |
| Hspa1b | Gm11808 | Il10 | Foxn3 | Pdcd4 | Gzma | Ly6c2 | Shisa5 |
| Hsp90aa1 | Uba52 | Sapcd1 | Stag2 | Abhd2 | Tyrobp | Ly6c1 | Ass1 |
| Hspa8 | Gm10073 | Tnfrsf4 | Igkc | Cbx3 | Fcer1g | Lcn4 | Tspan32 |
| Ubb | Gm9843 | Klrg1 | Chic2 | Wtap | Gzmb | Cd8a | Ecm1 |
| Gm26825 | Gm6133 | Penk | Arf6 | Ptp4a2 | Xcl1 | Ggt1 | Samhd1 |
| Jun | Gm9844 | Areg | Cnppd1 | Kdm6b | Tcrg-C4 | Ly6a | Cd4 |
| Dnaja1 | Gm26917 | Hmgb2 | Snapc3 | Mcmbp | Cd7 | Tmem108 | Ifi203 |
| Dnajb1 | Gm10036 | Cd83 | Sox4 | Notch2 | Dapk2 | Adgrb2 | Gimap3 |
| Klf6 | Gm2000 | Samsn1 | Pcgf5 | Gm26771 | Tcrg-C2 | Nkg7 | Igtp |
| Hsp90ab1 | Gm10263 | Ikzf2 | Asf1a | Fxyd4 | Cited4 | Glipr2 | Trav9-1 |
| Gnai2 | Gm9493 | Tnfrsf9 | Maea | Upf1 | Klra5 | Rcn3 | Stat1 |
| Grb2 | Grcc10 | Axl | Zfp330 | Actr2 | Klri2 | Thy1 | Ltb |
| Actg1 | Gm10116 | Batf | Ugcg | Marf1 | Cd244 | Gm43698 | S100a10 |
| Hsph1 | Nme2 | Dhrs3 | Tbc1d17 | B4galt1 | Tcrg-C1 | Ccr9 | Trav9d-1 |
| Fos | Gm10250 | Apoe | Smim19 | D17H6S53E | Zmat4 | Kcnc1 | Gpr83 |
| Lcp1 | Gm42418 | Ebi3 | Celf1 | Slc39a1 | Spry2 | Dapl1 | Cyp4f18 |
| Ppp2ca | Gm5093 | Smpdl3a | Mysm1 | Lrrc8a | Cd160 | B2m | Cytip |
| Dynll1 | Gm15013 | Nusap1 | 2210016L21Rik | Tmf1 | Gm156 | Slc25a4 | Capg |
| Jund | Gm8186 | Pou2f2 | Trip12 | Fn3krp | Klra7 | Klk8 | Cd2 |
| Calm1 | Gm6576 | Il17rb | Ubn1 | Alkbh5 | Klrc2 | Plac8 | H2-D1 |
| Glycam1 | Ddit4 | Dusp1 | Pten | Zbtb40 | Ccl4 | Racgap1 | Gimap1 |
| Slc3a2 | AY036118 | Ldlrad4 | Tmem128 | Ncoa3 | Lgals3 | Hcst | Cish |
| Srpk2 | Gm7808 | Mmd | Vps41 | Paip1 | Batf3 | Eomes | Gm5424 |
| Pitpna | Ighg3 | Nfkb1 | Chmp3 | Arglu1 | Klre1 | Runx3 | Trav9n-1 |
| Phlda1 | Psme2b | Rora | Cant1 | Zc3hav1 | Trdc | Tagln2 | Gata1 |
| Hspb1 | Gm10320 | S100a4 | Pafah1b1 | Kmt2d | Klre1 | Ppic | St8sia6 |
| Cacybp | Gm8797 | Cybb | Sun2 | Acot2 | Cd200r2 | Ifitm10 | Gbp7 |
| Fabp2 | Eno1b | Rilpl2 | Etnk1 | Rbm15 | Klra3 | Ms4a4c | Vim |
| Tra2b | Clca3a1 | Podnl1 | Gramd1a | Dpysl2 | Spp1 | Eng | Txnip |
| Baz1a | Ttc7 | Tnfaip8 | Mtf2 | Ppm1h | Ncr1 | Tm6sf1 | Iigp1 |
| Slc38a2 | Gm4950 | Hey1 | Mknk2 | Ppp1r16b | Lrrk1 | Ostf1 | Zbp1 |
| Gm42670 | Gm17669 | Il1rl1 | Nufip1 | Rap2b | Ckm | Lman1l | Cyb5a |
| Gm37170 | mt-Atp8 | St6galnac3 | Gnpnat1 | Pitpnc1 | Clnk | Adgrg5 | Tnfrsf18 |
| Tra2a | Gm5786 | Epas1 | Hagh | Inpp4a | Htra3 | Ehd1 | Tnfrsf25 |
| Cd40lg | Gm5239 | Ltb4r1 | Dock8 | Inafm2 | Klra9 | Saraf | Arl6ip1 |
| Cdk2ap1 | Lfng | Gm3636 | Armt1 | Skil | Klri1 | Osbpl6 | Rtp4 |
| Ccdc117 | Gm4978 | Atp6v0d2 | Cdk11b | Ptpn22 | Itga1 | Dnajc15 | Phf11b |
| Fabp6 | Lhfpl4 | Rab37 | Ap1s1 | Kdelr2 | Fgl2 | Slco3a1 | Nxpe3 |
| Nav1 | Gm16519 | Nt5e | Trmt2a | Lyrm4 | Car2 | mt-Nd1 | Ccdc22 |
| Taf10 | Lrrc58 | Trib1 | Arl8b | Otub1 | Trgv2 | Tapbpl | 1110032F04Rik |
| Ighg2c | Snhg9 | Wisp1 | Ldlrap1 | Mon1b | Gm16602 | Sorl1 | Adgre5 |
| Fabp1 | Gm10020 | Bhlhe40 | Wdr3 | Hsf2 | Emilin2 | AB124611 | Chd3 |
| Mrpl38 | Rnaset2a | Krt18 | Rbbp6 | Insig1 | Slc16a11 | Psme1 | Arhgap15 |
| Slc25a20 | Gm5426 | Cd44 | Stxbp2 | Slc24a5 | Gm19590 | Trim30c | Cd81 |
| Rabggtb | Ost4 | Ckap2l | Ppm1b | Zscan26 | Cd3g | Fcgrt | Znrf1 |
| Ahsa1 | Usp50 | Lztfl1 | Kif2a | Sema4b | 1810041H14Rik | G0s2 | Fsd1l |
| Swt1 | Gm43062 | Trappc1 | Pias1 | Ric1 | Ccl3 | 5730508B09Rik | Spcs2 |
| H60b | Gm10094 | Tnfsf8 | Txndc9 | 4933434E20Rik | Sh2d1b1 | Sec61b | Slfn1 |
| Ighv1-14 | Rtn4rl1 | Zfp36l1 | Chchd7 | Tc2n | Cyp2j6 | Sidt1 | Trav2 |

H. B cells PP

| topic_1 | topic_2 | topic_3 | topic_4 | topic_5 | topic_6 | topic_7 |
|---|---|---|---|---|---|---|
| Plac8 | Crip1 | Malat1 | Npm1 | H2-Ab1 | 2810417H13Rik | Actb |
| H2-Aa | Aicda | Xist | Mif | Cd74 | Stmn1 | Klf2 |
| Capg | Chrna9 | mt-Atp6 | Gm8730 | Ifi30 | Ube2c | Junb |
| Tmsb4x | sypl | mt-Co3 | Eef1g | H2-Eb1 | Cdca8 | H3f3b |
| S100a11 | Rgs13 | mt-Co1 | Ybx1 | Pfn1 | Ccnb2 | Fos |
| Cd79a | Lipc | mt-Cytb | Pebp1 | Il4i1 | Nusap1 | Ier2 |
| S100a10 | Scimp | mt-Co2 | Hspe1 | Tnfrsf13c | Tubb5 | Jund |
| Ly6e | Laptm5 | Rsrp1 | Hsp90ab1 | Marcksl1 | H2afz | Btg2 |
| Sapcd1 | Sorbs2 | Siglecg | Ranbp1 | Swap70 | Tuba1b | Nr4a1 |
| Cd38 | Pold4 | mt-Nd4 | Erh | Hspa1a | Tpx2 | Ppp1r15a |
| Gm9843 | Eaf2 | Ebf1 | Gm10709 | Syngr2 | Hmgb2 | Dusp1 |
| Arhgap24 | Arpc3 | Ptprc | Nme1 | Ptpn6 | Ccna2 | Cd69 |
| Gm6133 | Anxa2 | Rbm39 | Cox7c | Actr3 | Birc5 | Btg1 |
| Edaradd | Bfsp2 | Snx29 | Gm10260 | Cfl1 | Hist1h2ap | Rgs2 |
| Cyp4f18 | Gm3336 | Gm42726 | Hint1 | Hvcn1 | Kif22 | Tsc22d3 |
| Marcks | Eif3h | Mbnl1 | Atp5b | Cd40 | Plk1 | Zfp36 |
| Gm11808 | Slpr2 | mt-Nd1 | Snrpd2 | Grap | Cep55 | Pxdc1 |
| Cd72 | Prr13 | Ttc14 | Gapdh | Myl12a | Esco2 | Cxcr4 |
| Ostf1 | Gm32200 | mt-Nd2 | Atp5e | Dnaja1 | Asf1b | Dusp2 |
| Ccr1 | Gh | Atrx | Slc25a5 | Cyba | Cdca2 | Jun |
| Gm9493 | Neil1 | Chd6 | Eef1b2 | Samsn1 | Pbk | Ier5 |
| Sh3bgrl3 | Fth1 | Fchsd2 | Eno1 | Ms4a1 | Ptma | Myc |
| Fcmr | Ube2d2a | Mbnl2 | Gm10076 | Iep1 | Nuf2 | Stk17b |
| Fcrl1 | Smco4 | Klhl24 | Atp5g1 | Cd53 | Cdc20 | Kdm6b |
| Ifi203 | Dap | PISD | Prdx1 | Mif4gd | Rrm2 | Rhob |
| St3gal6 | Serf2 | Ddit3 | Chchd2 | Hnrnpf | Cdca5 | Ccr7 |
| Bcl2 | Coro1a | Rbm5 | Npm3 | Actg1 | Hmmr | Egr1 |
| Rdx | Kcng2 | Tsix | Phgdh | Slc25a19 | Top2a | Hist1h1c |
| Hhex | Ildr1 | Cdkal1 | Hnrnpa1 | Grb2 | Cenpe | Nfkbid |
| Sub1 | Pla2gl6 | Trim7 | Eif3i | Hcls1 | Neil3 | Satb1 |
| Sla | Cd79b | Arid4a | Pkm | Irf8 | Sgol1 | Plk2 |
| D1Ertd622e | Clta | Tra2a | Eef1d | Nfkb2 | Ckap2l | Klf6 |
| Serpinb1a | Bzw2 | Cwf19l2 | Gnb2l1 | Rab35 | Bub1b | Vps37b |
| Hck | Stbd1 | mt-Nd5 | Uqcrq | H3f3a | Mxd3 | Il2rg |
| Ephx1 | Oaz1 | Map3k14 | Nop58 | II21r | Ccnb1 | Sdc4 |
| Eef1a1 | Smim14 | Dock11 | Tpi1 | Trp53 | Hmgb1 | Srsf5 |
| Mndal | Dram2 | Msl2 | Snrpf | Cotl1 | Cks1b | Nr4a2 |
| Ncf1 | Commd4 | Prpf38b | C1qbp | Zfp362 | Cdca3 | Ptp4a2 |
| Pml | Apobec1 | Zfp280d | Cox5a | Bzw1 | Cdk1 | Tgif1 |
| Ypel3 | Cpne5 | Son | Fkbp1a | Apex1 | Lmnb1 | Gm26532 |
| H2-D1 | Gnb4 | Scaper | Uqcr10 | Vasp | Cdkn3 | Rrad |
| Zeb2 | Sept1 | mt-Nd3 | Serbp1 | Eif5 | Spc24 | Egr2 |
| Tpt1 | Tcea1 | Prpf4b | Ndufb2 | Calr | Tk1 | Rel |
| Trps1 | Rhoh | Safb | Ndufab1 | Arpc4 | Bub1 | Zfp36l2 |
| Fcrla | Smagp | Gm43291 | Mdh2 | Arpc1b | H2afx | Abhd2 |
| Gns | Glrx3 | Ate1 | Shmt2 | Nfkbia | Spag5 | Fosb |
| BC094916 | Borcs8 | Cspp1 | Phb2 | Erp44 | Sgol2a | Cytip |
| Cd300lf | Nuggc | Slc4a1ap | Hspd1 | Lyl1 | Melk | Gpr183 |
| Gm2a | Gm2447 | Ncor1 | Cox6c | Psme1 | Cdc25c | Atf4 |
| Slfn2 | Mroh7 | Sumf1 | Pa2g4 | Relb | Rad51ap1 | Neat1 |

| topic_8 | topic_9 | topic_10 | topic_11 | topic_12 |
|---|---|---|---|---|
| Ifi27l2a | Chd4 | Igkc | Iglc1 | Ighg2b |
| Ptms | Myo1c | Igha | Ighm | Ighg3 |
| Gm9844 | Ythdf2 | Ccr10 | lgkv5-39 | Ighg1 |
| Med11 | Fam173b | Jchain | Iglc2 | Ighg2c |
| Cdk2ap2 | Ttc1 | Hsp90b1 | Iglv1 | Igkv3-7 |
| Pkp3 | lsg20l2 | Tnfrsf17 | Iglv3 | Igkv4-72 |
| Phf14 | Apopt1 | Edem1 | Mzb1 | Igkv4-63 |
| Cd24a | Birc2 | lgkv4-59 | Six4 | Igkv6-25 |
| Fkbp8 | Pptc7 | Krtcap2 | Cst3 | Igkv5-48 |
| Mrpl10 | Mmgt2 | Fkbp11 | Apoe | Igkv4-53 |
| Lsg1 | R3hdm1 | Ssr4 | Il1b | Igkv3-4 |
| Hp1bp3 | Kdm2b | Cd28 | Prg2 | Igkv1-135 |
| Trmt10c | Sgf29 | Iman1 | Klk4 | Igkv4-86 |
| Ppp2r1a | Slc15a3 | Edem2 | Igkv2-109 | Igkv8-30 |
| Sdhaf4 | Srsf1 | Ssr3 | Gm11772 | Igkv1-110 |
| Tcirg1 | Exoc2 | Ckap4 | 8030442B05Rik | Igkv3-5 |
| Cstf3 | Oxr1 | Sdc1 | Clstn3 | Igkv4-68 |
| Ggta1 | Mtf1 | H13 | Cacna1s | Slpi |
| Pum2 | Asb7 | Pdia4 | Gm42917 | AY036118 |
| Vamp8 | Naa35 | Spcs2 | Iglc3 | Gm42418 |
| Parvg | Hif1a | Klrg2 | Ighv7-1 | Lyz2 |
| Nelfb | Acot9 | Igkv10-94 | Kctd17 | Gm37511 |
| Acot8 | Cltc | Mgat3 | Dnajb9 | Igkv8-24 |
| Szrd1 | Snx15 | Khdc1c | Chst1 | Cxcl13 |
| Rab5c | Ado | Kdelr3 | Fam174b | Gm26917 |
| Ddx54 | Pop7 | Anxa8 | Gm28062 | Cxcl9 |
| Faim | Mrpl3 | Gm35202 | Col5a1 | Igkv4-60 |
| Mettl5 | Ctu2 | Nacc2 | Tarm1 | Ly6c1 |
| H2-T22 | Nxf1 | Ccr9 | Gabrr1 | Ighe |
| Oat | Arv1 | Sec24d | Gm28513 | Clu |
| Slu7 | Tbl1xr1 | Mpzl2 | Sirpb1c | Fabp2 |
| Alyref2 | Leng8 | Trp53inp1 | Wnk2 | A230065H16Rik |
| Pex19 | Gon4l | Gtf2a1l | Prph | Ly6c2 |
| Ndufa8 | Nagk | Prss57 | Gm5103 | Ighv1-76 |
| Aco2 | Wdr6 | Ighv3-5 | D6Ertd527e | Gm11175 |
| Apls3 | Dynlt1c | Ly6g | Epyc | Fabp1 |
| Atg101 | Sema4d | Pls1 | Cdh19 | Igkv10-96 |
| Il16 | Lpcat3 | Entpd1 | Ceacam18 | Mfge8 |
| Rinl | Tmx2 | Ppib | Asb16 | Ighv1-62-1 |
| Zdhhc7 | Siah1a | Spcs1 | 1700011l03Rik | Ifitm3 |
| Aftph | Fam53a | Pla2g5 | Casq2 | Ighv11-2 |
| Slc30a9 | Atxn2l | Fkbp2 | Fam71b | Igkv6-23 |
| Necap2 | Ufm1 | Lgals1 | Lhfpl2 | Mmp25 |
| Tmem59 | Gm5900 | Fkbp14 | Gm17590 | Serpina1c |
| Prkcd | Ehmt2 | Mpo | Slc6al9 | Tmem176b |
| Hnrnph2 | Bsdc1 | Hoxd3os1 | Cplx1 | Igkv3-1 |
| Scly | Zfp628 | Nodal | Slc9a4 | Igkv4-71 |
| Oaz2 | Sp110 | Gm43121 | Abcd2 | Ccl2 |
| Napg | Dhrs3 | Txndc5 | Inmt | Igkv4-50 |
| Dync1li1 | Polb | Gm15533 | Xrra1 | Serpina1a |

I. DCs PP

| topic_1 | topic_2 | topic_3 | topic_4 | topic_5 | topic_6 | topic_7 |
|---|---|---|---|---|---|---|
| D13Ertd608e | Cst3 | Actb | mt-Co3 | S100a4 | Malat1 | Fscn1 |
| Cox6a2 | Xcr1 | Pfn1 | mt-Co1 | H2-DMb2 | Hspa1a | Ccl22 |
| Ly6c2 | Naaa | Cfl1 | mt-Atp6 | Napsa | Hspb1 | H2-M2 |
| Bst2 | Aif1 | Ndufa1 | mt-Co2 | Cd74 | Gm26825 | Nudt17 |
| Klk1 | Tlr3 | Cox6c | mt-Nd1 | Ltb | Gm42726 | Ccl5 |
| Iglc3 | H2-Aa | Myl6 | mt-Cytb | Ifi30 | Arhgap30 | Ccr7 |
| Ctsb | Cd207 | Arpc4 | mt-Nd2 | Sucnr1 | Pnisr | Cacnb3 |
| Ly6c1 | Ppt1 | Atp5j2 | mt-Nd4 | Cfp | Hspa1b | Crip1 |
| Cd8b1 | Fam149a | Cox6b1 | Tpt1 | Cyp4f16 | Prpf38b | Lad1 |
| Slpi | H2-DMbl | Mif | mt-Nd3 | Pglyrp1 | Rbm25 | Prg3 |
| Grn | Tlr11 | Myl12a | Fau | Gsn | Ubb | Tmem123 |
| Rnase6 | P3h2 | 2010107E04Rik | Wnk1 | Limd2 | PISD | Il4i1 |
| Klk1b27 | Cd52 | Psmb3 | Pabpc1 | Cdh17 | Swt1 | Tnfrsf4 |
| Siglech | Vwf | Usmg5 | Eif3f | Clec4a2 | Rsrp1 | Tmprss11d |
| Irf8 | Sult1a1 | Capzb | Map3k1 | Mdh2 | Dnajb1 | Socs2 |
| Cd209d | A530099J19Rik | Uqcr11 | Cirbp | Lpl | Gls | Gm16685 |
| Ctsl | 1700009J07Rik | Arpc5 | Zfp106 | S100a6 | Ankrd11 | Tnfrsf11b |
| Fcrla | Cxx1a | Arpc1b | Traf4 | Rasgef1b | Ankrd61 | Epsti1 |
| Snx5 | Eef1b2 | Crybb2 | Mbnl1 | H2-Oa | Tmem55b | Tspan3 |
| Sh3bgr | Cxx1b | Atp5e | Cdv3 | H2-Ob | Rnf152 | Nptx2 |
| Serp1 | Plpp1 | Tma7 | Eef2 | Stk32c | Zfp280d | B2m |
| Pir | Hepacam2 | Arhgdib | Xist | Gbx1 | 4833420G17Rik | Il12b |
| Upb1 | RP24-416M6.5 | Mrps21 | Mir142hg | Asphd1 | Golga1 | Tdh |
| Gm5547 | Psmb9 | Cox7b | Fam107b | Tmem141 | Tspyl2 | Aire |
| Scimp | Fgd2 | Ubl5 | Ddx3x | Prdx6 | Gm26522 | Slc1a3 |
| Pgls | Acvrl1 | Ost4 | Eef1a1 | Siglecg | Ogt | Insm1 |
| Dnajc7 | BC028528 | Lrrc58 | Stat3 | Arhgef2 | Bdp1 | H2-K1 |
| Smim5 | Eif3k | Serf2 | Gltscr2 | Tmem132c | Phip | Anxa3 |
| Cybb | Stfa2l1 | Tomm7 | Sp140 | Ggta1 | Eif4a2 | Lamp1 |
| Lair1 | Gramd2 | Ppp1r18 | Gnb2l1 | S100a5 | Gm42787 | Cd63 |
| Mpeg1 | Gatm | Rab5c | Skil | Gabarapl2 | Clk4 | Nid1 |
| Lefty1 | Mctp1 | Arpc2 | Tacc1 | Fbrsl1 | Dhx30 | Eno2 |
| Pld4 | H2-Ab1 | Chchd2 | Dock2 | Ppp1r14a | Gdap10 | Ankrd33b |
| Ly6a | Fuca1 | Ndufv3 | Kxd1 | Ffar2 | Lpin2 | H2-D1 |
| Nucb2 | E030013l19Rik | Psmc5 | 4933434E20Rik | Rgs12 | Zfp638 | Syt1 |
| Atp2a1 | Rab32 | Smdt1 | Zfos1 | Clec4a4 | Smg1 | Eno3 |
| P2ry14 | Cd36 | Cox6a1 | Aes | Lcp1 | Akap9 | Dkk2 |
| Ptprcap | Dnasel13 | Vapa | Cox7a2l | Lysmd2 | Tor1b | Aard |
| Ccr9 | Ece1 | Ufm1 | Defa24 | M6pr | Gm28707 | Zmynd15 |
| Dap | Khk | Pdrg1 | Gm26853 | Syngr2 | Tor4a | Slc7a10 |
| Sell | Naga | Gm15848 | Use1 | Rp2 | Akap8 | Id2 |
| Paqr5 | Tifab | Arpc5l | Usf2 | Slc6a13 | Dhx36 | Iscu |
| Robo3 | Olfm1 | Pfdn2 | Lrba | Gdi2 | Cspp1 | Cd200 |
| Klra17 | Naalad2 | Ndufa2 | Serinc1 | Rogdi | Hsp90aa1 | Efnb2 |
| Fbxl13 | Gm28411 | Fdps | Tomm20 | Sh3bp1 | Uvssa | Asprv1 |
| Sema4b | Proser2 | Gpx1 | Csde1 | Dlc1 | Trim24 | Prg2 |
| Myh13 | Tnfrsf22 | Gpsm3 | Whsc1l1 | Chst10 | Kdm3b | Tmem158 |
| Cdh5 | Rnf144b | Wdr1 | Amnl | Myo1b | Nxf1 | Tm4sf5 |
| Blnk | Tbxas1 | Akr1a1 | Dennd1b | Nav1 | Tyk2 | Slco5a1 |
| Lifr | Zfp652os | S100a10 | Gm8730 | Sirpa | Wdr13 | Hgd |

| topic_8 | topic_9 | topic_10 | topic_11 | topic_12 |
|---|---|---|---|---|
| Ptma | AY036118 | Ccl4 | Il1b | Ccl21a |
| Stmn1 | Gm26917 | Cel3 | Lyz2 | Nfkbia |
| 2810417H13Rik | Uba52 | Gpr171 | Plet1 | Mcpt1 |
| Tmsb10 | Igkc | Plaur | Il22ra2 | Dusp2 |
| Ccnb2 | Gm42418 | Fth1 | Ifitm6 | Junb |
| Vim | Gm10073 | H3f3b | Ifitm3 | Cd7 |
| Ranbp1 | Jchain | Klf2 | Atox1 | Tmem176b |
| Ube2c | Gm10076 | Hist1h1c | Gp2 | Sox4 |
| Ran | mt-Atp8 | Cd69 | Csf1r | Bel2 |
| Cks1b | Hist2h2aa1 | Jund | Ccr1 | Mcpt2 |
| Anp32b | Gm10260 | Ncf1 | Ass1 | Ier2 |
| Rrm2 | Gm21762 | Rilpl2 | Tarm1 | Trbc1 |
| Hmgb2 | Gm10116 | Fos | Mcemp1 | Shisa5 |
| Hmgb1 | Gm11175 | Btg1 | Srgn | Dusp1 |
| Cdca7 | Igkv4-72 | Herpud1 | Sirpb1c | Xcl1 |
| Tubb5 | Nme2 | Stk17b | Epcam | Mfge8 |
| Tuba1b | Igha | Ubc | Tnni2 | Nrgn |
| Dut | Gm20721 | St8sia4 | Csf2rb | Igfbp7 |
| Cenpm | Gm8797 | Neat1 | Ctsc | Dcn |
| Cdk4 | Igkv4-63 | Tsc22d3 | Mmp12 | Apoe |
| Pa2g4 | Gm28727 | Gm26532 | Sec61b | Spry2 |
| H2afx | Gml0020 | Cxcr4 | Lgals3 | Tnfsf11 |
| Cdkn3 | Gstp1 | Tsc22d1 | Msrb1 | Ifitm1 |
| Snrpd1 | Hist1h2a1 | Sub1 | Dab2 | Trbc2 |
| Birc5 | Eno1b | Irf1 | Igsf6 | Wfdc18 |
| Hnrnpf | Gm9493 | Ier5 | Havcr2 | Cd82 |
| 2700094K13Rik | Igkv3-4 | Cytip | Ear2 | Tmem176a |
| Ppia | Ighv11-2 | Fam46c | Itgax | Il18r1 |
| Mcm7 | Gclm | Ptger4 | Clec4n | Rorc |
| Nusap1 | Grcc10 | Evi2a | Fgl2 | Cyr61 |
| Srsf2 | Gm6576 | Zfp36l1 | Csf2ra | Hilpda |
| Eif5a | Igkv4-68 | 5430427O19Rik | Acp5 | Ikzf2 |
| Impdh2 | Dynlt1b | Zfp36l2 | 9530059O14Rik | Ighv1-85 |
| Srsf3 | Iglv1 | Zc3h12c | Ctsz | Hes1 |
| Npm1 | Gm11127 | Dyrk3 | Adgre1 | Prr7 |
| Rbm3 | 1810009A15Rik | Slc3a2 | Gm5150 | Ccl19 |
| Nudc | Igkv3-7 | Lca5l | Pilra | Gem |
| Nel | Igkv5-48 | Smc6 | Hfe | Cited4 |
| Pttg1 | Cd79a | Jakmip1 | Sat1 | Cd37 |
| Erh | Ighv9-1 | Il12a | Sirpb1b | Dkkl1 |
| Mcm2 | Gm9843 | Dusp18 | Agpat4 | Ddit4 |
| Snrpf | Ighv5-9 | Mef2c | Rrad | Chd3 |
| Mcm6 | Gm17275 | Dusp5 | Eps8 | Igfbp4 |
| Mcm3 | Tespa1 | Arrdc3 | Tmsb4x | Nr4a1 |
| Dnph1 | Sft2d1 | Ldlrad4 | Gngt2 | Cd3g |
| Banf1 | Pgk1 | Incpint | Gm2a | Nedd4 |
| Hnrnpk | Fabp2 | Ddit3 | Timd4 | Ier3 |
| Mcm5 | Gm26809 | Rab7b | Cd86 | Csf2 |
| Pcna | mt-Nd4l | Fam174a | AF251705 | Pim1 |
| Ccnb1 | Gm8186 | Ifnar1 | Fcer1g | Tgif1 |

J. Myeloid cells PP

| topic_1 | topic_2 | topic_3 | topic_4 | topic_5 |
|---|---|---|---|---|
| Ifitm3 | Eef1a1 | S100a9 | Cpa3 | Mcpt1 |
| Eyz2 | Gm8730 | S100a8 | Mcpt4 | Mcpt2 |
| Ey6c2 | Fau | G0s2 | Cma1 | Mcpt9 |
| Ace | Gm10709 | Cxcl2 | Cyp11a1 | Cd63 |
| S100a4 | Eef2 | Retnlg | Gata2 | Ifitm1 |
| Ear2 | Actb | Gm5483 | Tpsb2 | Cma2 |
| Plac8 | mt-Atp6 | Il1b | Il4 | Pga5 |
| Ly6c1 | Cox4i1 | Cxcr2 | Hs3st1 | Gm10354 |
| Gm15987 | Npm1 | Lrg1 | Il1rl1 | Speer4e |
| Thbs1 | Eif3h | Ecn2 | Ubb | Prnp |
| Ms4a4c | Sh3bgrl3 | Asprv1 | Ccl4 | Itgae |
| Chil3 | Tpt1 | Stfa2l1 | Dnajb1 | Ldhc |
| Clec4a3 | Rbm3 | Il1f9 | Furin | Gm2a |
| Clec4a1 | Gapdh | Wfdc21 | Rab27b | Sec14l2 |
| F13al | Naca | Msrb1 | Cited4 | Gm21190 |
| Ifitm6 | Eif3f | Marcksl1 | Il13 | Ctla2a |
| Ifi27l2a | Eef1b2 | Mrgpra2b | Jun | Itgb6 |
| Cst3 | Eif3e | S100a11 | Srgn | Apol9a |
| Sat1 | Pabpc1 | BC100530 | Ccl2 | Gzmc |
| Ccl9 | Atp5e | Chil1 | Fos | Htra1 |
| Lrp1 | Wdr89 | Trem1 | Ccl3 | Tjp1 |
| Ptpro | Pfdn5 | Csf3r | Slc45a3 | Cttn |
| Adgre4 | Ppib | Mmp9 | Klk8 | Adgrg1 |
| Cybb | 2010107E04Rik | Fth1 | Egr1 | H19 |
| Ifi205 | Arhgdib | Clec4e | Junb | 5830473C10Rik |
| Pid1 | Atp5l | Irg1 | Serpine1 | Ldlrad4 |
| Colec12 | Cox5b | Steap4 | Gchfr | Hey1 |
| Ryr1 | Gpi1 | Clec4d | Itm2c | Ddit4 |
| Rap1a | Atp5h | Tnfaip2 | Cyp26a1 | Cpne5 |
| Nupr1 | Atp5d | Pglyrp1 | Pdcd1lg2 | Gm17019 |
| Tspan17 | Ssr4 | Il1rn | Rgs1 | Apol9b |
| App | Polr1d | Hcar2 | Chst1 | Tspan8 |
| Ifngr1 | Eif3k | Mxd1 | Fcer1a | Ikzf4 |
| Mpp1 | Tomm7 | Cdk2ap2 | Slc24a3 | Nphs2 |
| Hpse | Psmb1 | Cxcl3 | Angpt1 | Speg |
| Pira2 | Serf2 | Il1r2 | Glul | Al838599 |
| Rhou | Cox7a2l | C5ar1 | Serpinb1a | Abca13 |
| BC028528 | mt-Co2 | Stfa2 | Fosb | Irrc36 |
| Pparg | mt-Nd1 | Gadd45b | Tnfaip8 | Atp13a4 |
| Slc12a2 | Aldoa | Slc16a3 | Plgrkt | Asb17os |
| Ms4a8a | Atp5f1 | Dusp1 | Cited2 | Gm9758 |
| Nfil3 | Sec61b | Rasip1 | Itm2b | Otop1 |
| Arl5c | Atp5o | Cd9 | Hs6st2 | Gdf11 |
| Gm14548 | Acbd6 | Cebpb | Zmat4 | Mcam |
| Smpdl3a | Ppard | Slfn1 | Nfkbid | Tmem143 |
| Cd300e | Npm3 | Cd300If | Adamts9 | Zbtb49 |
| Sowahe | Uqcrh | Mrgpra2a | Rfc2 | Fam151a |
| Gpx1 | Ndufa6 | Ly6g | Tsc22d1 | Fermt2 |
| F10 | Gnb2l1 | Cyp4f18 | Creb3l1 | Ccnb1ip1 |
| Tifa | Tma7 | Pilra | Rgs18 | Tchp |

| topic_6 | topic_7 | topic_8 | topic_9 | topic_10 |
|---|---|---|---|---|
| Gzmb | Prss34 | Cd74 | Cdkn1a | Uba52 |
| Malat1 | Mcpt8 | H2-Aa | Bhlhe40 | Gm26917 |
| Slco2b1 | Lgals1 | H2-Eb1 | Hnrnpll | AY036118 |
| Fndc5 | Ran | H2-Ab1 | Hist1h1c | Gm42418 |
| Xist | Stmn1 | C1qa | Chmp5 | Gm10076 |
| Hes1 | Akr1c18 | C1qb | Uap1 | Tmsb10 |
| Psd3 | Ube2c | C1qc | Hint2 | Gm10073 |
| Gnaz | Ms4a3 | Apoe | Ccdc107 | Gm10116 |
| Gm11697 | Erh | H2-DMb1 | Txnl4a | mt-Atp8 |
| Srsf5 | Ptma | Pla2g2d | Kmt2e | Igkv4-72 |
| Bfsp2 | Nme1 | H2-DMb2 | Maz | Vim |
| Lmo4 | Ranbp1 | Tmem176a | Srsf4 | Nme2 |
| Gm12840 | Cdca3 | Cd81 | Mrps24 | Eno1b |
| Rnf19b | Cdca8 | H2-DMa | Ppm1g | Crip1 |
| Zbtb20 | Nusap1 | Tmem176b | Eif4g2 | Gm10260 |
| Fosl2 | Ppia | Cxcl9 | Hnrnpr | Gm11175 |
| Gm20186 | Ccna2 | Gm15056 | Ifrd1 | Hist2h2aa1 |
| Lag3 | Tmem160 | Mmp14 | Ahr | Klf2 |
| mt-Nd3 | Dut | Cxcl16 | Yif1a | S100a10 |
| Csrp3 | Anp32b | Spon1 | Zfp91 | Gm9844 |
| Susd6 | Eef1g | Dnase1l3 | Ankrd12 | Gm6576 |
| Smarca1 | Ap3s1 | Apol7c | Anapc5 | Gm17275 |
| Ccnd2 | Tomm20 | Lyz1 | Lcp2 | Il2rb |
| Nap1l5 | Dad1 | Acp5 | Ids | Ahnak |
| Rab4a | Nop10 | Il22ra2 | Alad | Snx22 |
| Tpbgl | Tacc3 | Prg3 | Stub1 | Ighv9-3 |
| Slc6a6 | Lsm6 | Aif1 | Nabp2 | Gm7676 |
| 4933407L21Rik | Parvg | Ms4a7 | Scamp1 | Gm20721 |
| Grik5 | Snrpd2 | Mrc1 | Sde2 | Gm10020 |
| Muc1 | Pgam1 | Gas1 | Wbp2 | Igkv4-86 |
| Ssbp3 | Uqcr11 | Kcnj10 | Vat1 | Gm9493 |
| Ccdc138 | Ndufa12 | Ctsh | Il22 | Gm8186 |
| Smo | Gng12 | Cp | Larp7 | Il23r |
| Tgfbr1 | Tubb5 | Ifi30 | Sec31a | Pgk1 |
| Zfp941 | Banf1 | Adamdec1 | Ralbp1 | Ccr6 |
| Ikzf2 | Sumo3 | Cd209b | Chchd3 | Kmo |
| Zfp638 | Cel6 | Timd4 | Tdg | Hnrnpa3 |
| Smyd4 | Timm17a | Axl | Ddb1 | Ighv11-2 |
| Rgs2 | Atp5g1 | Rab3il1 | Crem | Nr1d1 |
| Pdgfrb | Mrpl18 | Slc1a2 | Gpbp1l1 | Igkv3-5 |
| Zfr | 2810417H13Rik | Wnt6 | Trp53 | Gm28727 |
| Patz1 | Cyc1 | Cxcl14 | Hs6st1 | Sik1 |
| Fam120c | Srsf6 | Il27 | Vdac3 | Gstp1 |
| Akap13 | Tuba1b | Sdc4 | Neurl3 | Hmga1 |
| Ugcg | C1qbp | Clec9a | Hmces | Diaph1 |
| H3f3b | Klk4 | Apo110b | Matr3 | Styx |
| Cx3cr1 | Ppan | Lgmn | Cmas | Gm11127 |
| Dnaja1 | Cdk1 | Cabp4 | Gsk3b | Tnfsf13 |
| Stard10 | Anp32e | Nuak1 | Wtap | Gm10146 |
| Fam129c | Dkc1 | Tlr12 | Dcun1d5 | Sft2d1 |

K. ILCs PP

| topic_1 | topic_2 | topic_3 | topic_4 | topic_5 | topic_6 | topic_7 |
|---|---|---|---|---|---|---|
| Lgals3 | Shisa5 | Cel5 | Il22 | Xcl1 | Rgs1 | Hes1 |
| Slc6a20a | Ltb | Gzma | S100a4 | Gimap4 | Txk | Calca |
| Ffar2 | Bel2 | Klrel | Ode1 | Bcl2a1b | Akap13 | Il13 |
| Upp1 | Tmem123 | Klra3 | Piwil4 | Ctsw | Itch | Hs3st1 |
| Lrrn2 | Eif3l | Ccl4 | Parvb | Cd52 | Hist1h1c | Areg |
| Serpinb1a | Fam173b | Klra9 | Cd83 | B2m | Aqr | Il4 |
| Ly6g5b | Trmt112 | Klra7 | S100g | Bcl2a1d | Smad7 | Cel1 |
| Ppp1r14c | Ciz1 | Ms4a4b | Sdc4 | Gpr55 | Plgrkt | Hilpda |
| C4b | Timm9 | Lgals1 | Hebp1 | Pfn1 | Wdr47 | Gata3 |
| Id2 | Limd2 | Tmsb4x | Il17f | Klrk1 | Mndal | Homer2 |
| Tgm3 | Blmh | Cel3 | Ptges | Hopx | Pum2 | Ccr4 |
| Pcp4 | Mat2b | Cd2 | Ldhb | Coro1a | March7 | Serpine1 |
| Ucp3 | Dpf2 | Gzmb | Rasl11a | Serpinb6b | Afg3l2 | Hba-a1 |
| Ptprz1 | Tia1 | Ctla2a | Ramp1 | Ikzf2 | Gimap8 | Vps37b |
| Rnase4 | H2-D1 | H2afz | Tnfsf11 | Ncr1 | Phf3 | Ptgir |
| Amical | Srsf1 | Nkg7 | Prr29 | Hcst | Gimap6 | Rxrg |
| Eps8l3 | Lias | AW112010 | Adra1b | Serpinb9 | Avl9 | Samsn1 |
| Rbpms2 | Cnih1 | Sh2d1a | Cldn10 | Cd160 | Tecpr1 | Il17rb |
| Actn2 | Supt4a | Klra1 | Hmgn3 | Il2rb | Il18r1 | Lmo4 |
| Tns4 | Spin1 | Klrd1 | Grb10 | Il4ra | Tcp11l2 | Il5 |
| Cdh10 | Poldip3 | Ifitm10 | Cryaa | S100a11 | Gramd1a | Deptor |
| Asb2 | Dctn2 | Eomes | Ccdc184 | Ptma | Foxj3 | Stxbp6 |
| Rax | Aspscr1 | Klra4 | Nrp1 | Sh3bgrl3 | Cdk12 | Dusp10 |
| Ccr9 | Zfp788 | Ly6c2 | Gpx1 | Ms4a6b | Fus | Lgals7 |
| Ryk | 2700094K13Rik | Itgam | Cd81 | Rbm3 | Irak2 | Scel |
| Cxcr6 | Csk | Anxa2 | Lta | Cox8a | Lgals8 | Ramp3 |
| Krt4 | Golga7 | Ctsd | Cntn1 | Lsp1 | Jakmip1 | Slc7a8 |
| Maf | Cd2bp2 | Irf8 | S100a6 | Klrb1f | Cdkn1b | Furin |
| Cd163l1 | Commd2 | Tm6sf1 | Cd82 | Ubald2 | Mthfs1 | Nfkb1 |
| Prelid2 | Rnf8 | Clip4 | Fam110a | Fasl | Mkln1 | AA467197 |
| Pabpc1l | Vars | Gfra2 | Mrfap1 | Cxcr3 | Hipk1 | Sox8 |
| Rinl | Serinc1 | Ctla2b | Fth1 | Phgdh | Usp48 | Sub1 |
| Ucp2 | Prmt3 | 1700025G04Rik | Foxs1 | Trac | Bclaf1 | Gm973 |
| Aqp3 | Mrpl15 | Klrb1c | Espn | Serpina3g | Dusp11 | Gm20186 |
| Krt79 | Ino80b | Rac3 | Igfbp7 | Irg1 | AU020206 | Cntnap2 |
| Ddx25 | Golga3 | Sema4a | Bst2 | Nabp1 | Atg12 | Pparg |
| Zfp414 | Rpe | Serpinb9b | Adm | Slc16a6 | Snrk | Frmd4b |
| S100a10 | Nfkbil1 | Cma1 | 5930412G12Rik | Cfl1 | Peli1 | Il9r |
| Hbegf | Rbm4b | Ugcg | Ube2e3 | Gm19585 | Galnt7 | Lrrc52 |
| Gstm5 | Tmx3 | Klra10 | Cpn1 | Psmb8 | Rbm34 | Spty2d1 |
| Gjb2 | Bloc1s4 | Sgk1 | M1ap | 1-Sep | Git2 | Ptpn13 |
| Klrb1b | Cct8 | Kcnj8 | Krt25 | Cd274 | Esco1 | Ccrl2 |
| Amhr2 | Zkscan3 | Ccr5 | Ostf1 | Ifngr1 | Zfp36l2 | Neb |
| F2r | Nudt18 | Ptprc | Nkx6-2 | Gimap3 | Mxd4 | Plaur |
| 6720489N17Rik | Polr2j | Ly6c1 | Ecm1 | Lck | Taf8 | Itk |
| Tg | Eif4a2 | Gramd3 | Ccr6 | Psme2 | Rhoh | Icos |
| Hs6st2 | Cs | Atp1b1 | S100a3 | Ckm | Ccdc91 | Crem |
| Prpsap1 | Nae1 | Ccr2 | Batf3 | Cd7 | Kdm6a | Esm1 |
| Ppt2 | Nono | Qrfp | Chad | St8sia4 | Nosip | Cxcl2 |
| Capg | Ikbkg | Samd3 | Mpzl1 | Nhp2 | St6galnac3 | Gpr65 |

| topic_8 | topic_9 | topic_10 | topic_11 | topic_12 |
|---|---|---|---|---|
| Cd74 | Junb | Gm8730 | AY036118 | Fos |
| H2-Aa | Gadd45b | Eef1a1 | Gm26917 | Dusp1 |
| Malat1 | Nfkbia | Gm10260 | Gm42418 | Jun |
| H2-Eb1 | Nr4a1 | Gm9493 | Uba52 | Ier2 |
| H2-Ab1 | Fosb | Gm9843 | Gm11175 | Egr1 |
| Hspa1a | Nfkbid | Gm10709 | mt-Atp8 | Zfp36 |
| Hspa1b | Btg2 | Gnb2l1 | Igkv4-72 | Klf6 |
| H2-DMb1 | Tgif1 | Gm11808 | Igkv3-7 | Rhob |
| H2-DMb2 | Tagap | Naca | Igkv4-63 | Klf2 |
| Gm26825 | Pim1 | Fau | Igkv3-4 | Ccl21a |
| Atrx | Ubb | mt-Co3 | Ighv9-3 | Cd69 |
| Mef2c | Phlda1 | mt-Co2 | Gm20721 | Tsc22d3 |
| Dnajb1 | Klf4 | Gm2000 | Ighv11-2 | Rgs2 |
| H2-DMa | Jund | Eef1b2 | Eno1b | Oser1 |
| Dcstamp | Ier5 | Npm1 | Gm11127 | Tnf |
| H2-Oa | Zfp36l1 | Eef1g | CAAA01147332.1 | Mcpt2 |
| Siglecg | Maff | Gm10036 | Igkv4-68 | Gm26532 |
| Iglc1 | Spry2 | Wdr89 | Igkv1-110 | Egr2 |
| Ly86 | Ubc | Eif3h | Gm8797 | Mcpt1 |
| Tnip3 | Btg1 | Gm6133 | Igkv3-5 | Thra |
| Cited4 | Hspa5 | mt-Co1 | Igkv6-25 | S100a9 |
| Ms4a4c | Gem | Uqcrh | mt-Nd4l | Zfp395 |
| Sacs | Pnrc1 | Cd3g | Gm10073 | Snhg12 |
| Srrt | Tuba1a | Hspe1 | Siglech | Ifitm1 |
| Golga4 | Mir142hg | Hnrnpa1 | Gnai2 | Lztfl1 |
| Napsa | Tgoln1 | Eif3e | 1810009A15Rik | Polg2 |
| Ctse | H1f0 | Izumo1r | Tsix | Pias1 |
| Zfp24 | Eif1 | Hspa8 | Gm28727 | H2-T23 |
| Shprh | Morf4l2 | Atp5a1 | Rbm39 | Kdm5a |
| Cacybp | Sertad1 | Eif3f | Ier5l | Birc3 |
| AC125149.3 | Atf4 | Cox4i1 | Igkv5-39 | 2900060B14Rik |
| Hspb1 | Tuba1c | Ccr7 | Lyz2 | Gm26802 |
| Esd | Gm17056 | Cct5 | Pde4b | Dusp6 |
| Arhgap30 | Gm10827 | Eif3k | Myl12b | Prrc2b |
| Iglc3 | Herpud1 | Tpt1 | Zg16 | Srpr |
| Gm12976 | H3f3b | Gm10269 | Iglv1 | Serpina1b |
| Trav11 | Sqstm1 | Hsp90ab1 | Gm10116 | Hs3st3a1 |
| Gm37170 | Tubb4b | Cox7a2l | Ighv5-4 | Mcpt4 |
| Cdadc1 | Bcl10 | Plac8 | Gclm | Chic2 |
| Igkv9-124 | Stk24 | Fbl | Gm17275 | 4833403J16Rik |
| Gstm1 | Plrg1 | Cct4 | Ighv9-1 | Fam21 |
| Ppp1r3d | Hnrnpf | Eif3m | Ndfip1 | Cox10 |
| Ltbp2 | Ensa | Btf3 | Ighv1-62-1 | Zfp322a |
| Natd1 | Tuba4a | Snrpe | Fabp2 | Tgds |
| Calcoco1 | Mgat4a | Atp5b | Nme2 | Etaa1 |
| 1700030J22Rik | Rgcc | Gm10263 | Ncor1 | Dyrk3 |
| Zzz3 | Usp37 | Gpi1 | Igkv5-48 | Amn1 |
| Gm6297 | Ddx3x | Anp32b | Coxl6 | Hsf1 |
| Lgi4 | Csf2 | Zfos1 | Rapgef6 | Hmox2 |
| Antxr1 | Atxn1 | mt-Atp6 | Psap | Tmem252 |

TABLE 5
The enriched peaks and their associated genes in the
intestinal KLRG1 + ILC2s stimulated in vitro with
α-CGRP or control, related to FIG. 6.

| | Peaks enriched in ILC2s treated | |
|---|---|---|
| Gene | with α-CGRP | |
| 0610009L18Rik | 84328 (−4512), 84336 (−139), | |
| | 84337 (+789), 84339 (+1590), | |
| | 84340 (+4057) | |
| 0610010K14Rik | 78877 | (−2199) |
| 1110008F13Rik | 16421 | (+331) |
| 1110037F02Rik | 25171 | (−31942) |
| 1110065P20Rik | 29118 | (−661) |
| 1600029D21Rik | 61961 | (−17707) |
| 1700011E24Rik | 115690 | (+143781) |
| 1700011H14Rik | 97385 | (+71505) |
| 1700017B05Rik | 62437 | (−5689) |
| 1700021F05Rik | 69422 | (−8) |
| 1700025G04Rik | 5808 | (+12) |
| 1700034J05Rik | 46595 | (+7037) |
| 1700040L02Rik | 70667 | (+254838) |
| 1700056E22Rik | 8006 (+165268), 8004 (+229579) | |
| 1700060C20Rik | 16591 (−8759), 16596 (+12303) | |
| 1700101G07Rik | 17691 | (−600954) |
| 1700106J16Rik | 80781 (+204591), 80785 (+232776) | |
| 1700112E06Rik | 96208 | (+879765) |
| 1810013L24Rik | 105927 | (+884) |
| 2310015B20Rik | 70735 | (−23766) |
| 2410127L17Rik | 121182 | (−31040) |
| 2610002J02Rik | 31919 | (−15877) |
| 2610507B11Rik | 79746 | (+526) |
| 2610524H06Rik | 36919 | (−936) |
| 2810025M15Rik | 6281 | (−31463) |
| 2810403A07Rik | 21226 | (+1528) |
| 3110018I06Rik | 89138 | (+7546) |
| 3110021A11Rik | 44783 | (+926) |
| 3110079O15Rik | 3284 | (+89139) |
| 3930402G23Rik | 54431 (−77281), 54430 (−76792), | |
| | 54412 (−66834), 54409 (−64079), | |
| | 54395 (−54062), 54392 (−53111), | |
| | 54391 (−52665), 54365 (+207770) | |
| 4833420G17Rik | 95571 | (−32739) |
| 4921513D11Rik | 114933 | (+72723) |
| 4921513I03Rik | 73819 | (+130219) |
| 4930404H24Rik | 16106 | (−48419) |
| 4930426L09Rik | 9753 | (−43) |
| 4930432M17Rik | 23061 | (+44725) |
| 4930438A08Rik | 77812 | (−35454) |
| 4930447C04Rik | 86799 | (+153671) |
| 4930524B15Rik | 75933 | (−75371) |
| 4930538K18Rik | 28775 | (+13346) |
| 4930597O21Rik | 42288 (−101856), 42274 (−819) | |
| 4931440P22Rik | 20165 | (+154) |
| 4932438H23Rik | 109571 (−4035), 109563 (+12493) | |
| 4933406P04Rik | 68275 | (+162741) |
| 4933415A04Rik | 76309 | (−420) |
| 5031425E22Rik | 32592 | (+17874) |
| 5033430I15Rik | 92261 | (+115889) |
| 5730507C01Rik | 85150 | (+95808) |
| 5830418K08Rik | 60145 | (−136432) |
| 5930422O12Rik | 55096 | (+88982) |
| 8430427H17Rik | 16137 (+355), 16136 (+809) | |
| 8430432A02Rik | 1106 | (−413) |
| 9230102O04Rik | 9123 | (−108534) |
| 9230109A22Rik | 101059 (−263217), 101021 (+4006) | |
| 9430020K01Rik | 115987 (+603), 115988 (+19036) | |
| 9930013L23Rik | 50858 | (−30655) |
| 9930021J03Rik | 121699 | (−559) |
| A1bg | 102424 | (+66950) |
| A230083G16Rik | 45188 | (+912) |
| A330050F15Rik | 114458 | (+1922) |
| A3galt2 | 29324 | (−31257) |
| A630091E08Rik | 51231 | (+28383) |
| A930004D18Rik | 9612 | (−10247) |
| A930011G23Rik | 35707 | (−44408) |
| AI118078 | 62238 | (+514) |
| AI314180 | 26741 (−30), 26740 (+518) | |
| AI481877 | 26785 | (−21190) |
| AI593442 | 62110 | (+491132) |
| AW554918 | 116568 | (+3191) |
| Abca1 | 26454 | (−36323) |
| Abca2 | 10018 (−136), 10019 (+412) | |
| Abcb1a | 32308 (+53254), 32311 (+63017), | |
| | 32314 (+115280) | |
| Abcb1b | 32308 (−84816), 32311 (−75053), | |
| | 32314 (−22790), 32323 (+41708) | |
| Abcb4 | 32323 (−53862), 32330 (+330) | |
| Abcc1 | 106302 | (+610) |
| Abcc3 | 81037 | (−80045) |
| Abcd3 | 23064 | (+78627) |
| Abhd12b | 86641 (−42098), 86642 (−36811) | |
| Abhd17c | 50858 | (+34736) |
| Abi1 | 9858 | (+877) |
| Ablim1 | 123468 | (−139686) |
| Abr | 79500 (−133068), 79499 (−114022) | |
| Acadl | 2425 | (−33360) |
| Acads | 36984 | (+27907) |
| Acer2 | 27375 | (+9351) |
| Acot1 | 87778 | (−10085) |
| Acot11 | 28098 (−36161), 28075 (+49934) | |
| Acot2 | 87778 | (+11556) |
| Acot7 | 31732 | (+998) |
| Acox2 | 95628 | (+43905) |
| Acoxl | 14900 | (+200492) |
| Acpp | 65638 | (+30745) |
| Acsl6 | 77479 | (−16096) |
| Actb | 39157 (+3897), 39156 (+4467), | |
| | 39153 (+5862) | |
| Actbl2 | 95067 | (+233930) |
| Actc1 | 14137 | (+1837) |
| Actg1 | 84340 (−4193), 84339 (−1726), | |
| | 84337 (−925), 84336 (+3), | |
| | 84328 (+4376), 84327 (+5002), | |
| | 84315 (+43313) | |
| Actn4 | 48096 | (−10570) |
| Actr3 | 4201 | (+8619) |
| Actr8 | 96616 (+13886), 96618 (+15186), | |
| | 96620 (+15722) | |
| Acvr1c | 11959 | (+169986) |
| Acvr2a | 11599 | (−404201) |
| Adam10 | 64023 | (+865) |
| Adam9 | 54836 | (−232) |
| Adamts17 | 49598 | (+272963) |
| Adamts20 | 104685 | (−2322) |
| Adamts14 | 21722 | (+21507) |
| Adarb1 | 71056 | (−1257) |
| Adck1 | 88222 | (+106440) |
| Adcy1 | 75018 (−395889), 75023 (−389427), | |
| | 75025 (−388199), 75026 (−387948), | |
| | 75027 (−387534), 75031 (−384813), | |
| | 75032 (−384150), 75034 (−382941), | |
| | 75035 (−381981), 75049 (−294451) | |
| Adcy9 | 105732 | (−36090) |
| Add1 | 33368 | (+1175) |
| Add2 | 42992 | (−3093) |
| Add3 | 123123 | (+1079) |
| Adnp | 17613 | (+89724) |
| Adnp2 | 119244 | (−47152) |
| Adora2a | 70877 (+3550), 70883 (+9781) | |
| Adprhl2 | 29227 | (+20268) |
| Aebp2 | 46123 (−2515), 46145 (+27408), | |
| | 46154 (+50763) | |
| Aff1 | 35968 (+737), 35989 (+102286) | |
| Aff3 | 805 | (−10925) |
| Aff4 | 77169 | (+903) |
| Agap1 | 3452 (−324213), 3462 (−123) | |
| Agap2 | 74129 | (+25249) |
| Agfg1 | 3005 | (+290) |
| Aggf1 | 94171 | (+45691) |
| Agl | 23011 | (−6108) |
| Agpat3 | 71188 | (−54322) |
| Ahdc1 | 29863 | (+1030) |
| Ahnak | 120568 | (+1978) |
| Aifm2 | 70260 | (−5342) |
| Akap12 | 67491 (−166178), 67489 (−154343), | |
| | 67472 (+118704), 67471 (+120447) | |
| Akap6 | 86127 (−55095), 86148 (+129795) | |
| Akap7 | 68683 (+2009), 68661 (+129124) | |
| Akirin1 | 29069 | (−46) |
| Akt2 | 47896 | (+880) |
| Aktip | 57460 | (+330) |
| Aldhla2 | 64058 | (−35278) |
| Alg13 | 125856 | (+208421) |
| Alg2 | 26376 | (+105378) |
| Alkbh2 | 36832 | (−2753) |
| Alkbh5 | 78047 | (+934) |
| Alpk3 | 50691 | (−62915) |
| Alpl | 30624 | (+88307) |
| Amfr | 57537 | (−563) |
| Amhr2 | 105535 | (−34087) |
| Amph | 90502 | (+5864) |
| Anapc10 | 56727 | (+139316) |
| Ank | 101132 | (+345) |
| Ankfy1 | 79054 | (−81509) |
| Ankrd1 | 122084 | (−229547) |
| Ankrd13a | 36902 | (−1131) |
| Ankrd13c | 24665 | (+15755) |
| Ankrd17 | 35316 | (−1041) |
| Ankrd44 | 1743 | (+82) |
| Ankrd50 | 19362 (+20660), 19352 (+111205), | |
| | 19347 (+204372) | |
| Ankrd52 | 74367 | (+302) |
| Ankrd55 | 95165 | (+151125) |
| Ankrd63 | 14313 (+900), 14312 (+1153) | |
| Ankrd9 | 89484 | (−109) |
| Anks6 | 26337 | (−186) |
| Ano10 | 67120 | (+129511) |
| Ano6 | 104731 | (+96795) |
| Ano8 | 56380 | (+8369) |
| Anp32a | 63022 | (+169356) |
| Anp32b | 26257 | (+1755) |
| Anxa11 | 96387 | (−11647) |
| Aoah | 90599 | (−701636) |
| Aoc2 | 82182 | (−1278) |
| Aox3l1 | 1853 (+25522), 1854 (+26224) | |
| Ap1ar | 23241 | (−133) |
| Ap3s1 | 117687 | (+1181) |
| Ap5b1 | 120131 (−292), 120132 (+140) | |
| Apbb2 | 34670 (−801), 34654 (+97117) | |
| Apoa1 | 61750 (−208151), 61758 (−181311) | |
| Apoh | 83155 | (−17034) |
| Apol8 | 103355 | (+203) |
| Apool | 125496 | (+43) |
| Apopt1 | 89594 | (+9634) |
| Aqp9 | 64058 | (−17222) |
| Arap1 | 51577 | (−4794) |
| Arap2 | 34198 | (−748) |
| Arcn1 | 61597 | (−62) |
| Areg | 35399 | (+1475) |
| Arfgef1 | 101 | (+214) |
| Arg1 | 68683 (−369685), 68661 (−242570) | |
| Arhgap12 | 116032 (+542212), 116031 (+542705) | |
| Arhgap17 | 52743 | (+151504) |
| Arhgap23 | 81476 | (−10297) |
| Arhgap27 | 82468 (−35972), 82452 (−743) | |
| Arhgap39 | 103308 | (−4968) |
| Arhgap5 | 86106 (−11528), 86127 (+128211) | |
| Arhgdia | 84386 (−86), 84385 (+473) | |
| Arhgef33 | 115026 | (+84450) |
| Arid1a | 30025 (−64017), 30018 (−788) | |
| Arid1b | 110306 (+736), 110308 (+1738), | |
| | 110309 (+2355) | |
| Arid2 | 104731 | (−399935) |
| Arid4a | 86713 (+727), 86714 (+1083) | |
| Arid5b | 70667 (−8332), 70620 (+151409) | |
| Arl5a | 11801 | (+38247) |
| Arl6ip4 | 38003 | (+71621) |
| Armc2 | 69276 | (+129756) |
| Arntl | 52206 | (+813) |
| Arrb1 | 51359 | (+914) |
| Arrdc4 | 49719 | (−152) |
| Art2b | 51617 | (+102758) |
| Asap1 | 102684 | (+48795) |
| Asap2 | 85161 (−35783), 85164 (+471) | |
| Asb17 | 24619 | (+5560) |
| Asb2 | 88929 | (+21768) |
| Ascc1 | 70055 | (−34060) |
| Ascl2 | 53960 | (+1067) |
| Ascl4 | 72076 | (+25046) |
| Aspg | 89642 | (+33750) |
| Asphd2 | 36675 | (+9705) |
| Asxl1 | 16106 | (+98526) |
| Atad2b | 84775 | (+628) |
| Atf1 | 105215 | (+545) |
| Atf4 | 103787 | (+3742) |
| Atg12 | 117687 | (−1519) |
| Atg14 | 97330 (−81294), 97318 (+43834), | |
| | 97317 (+45922) | |
| Atg4b | 3743 | (−78) |
| Atl1 | 86614 | (+42) |
| Atp10a | 49309 | (+703) |
| Atp12a | 97846 | (−88711) |
| Atp13a1 | 56058 | (+828) |
| Atp1a1 | 22210 | (+110520) |
| Atp1b1 | 6755 (−23507), 6753 (−389) | |
| Atp2b1 | 72899 (−44), 72900 (+559) | |
| Atp6v1a | 107942 | (+112880) |
| Atp6v1c2 | 85112 | (+57031) |
| Atp8b3 | 71468 | (+43050) |
| Atr | 65233 (−4464), 65236 (+900) | |
| Atrn | 15125 (−62), 15126 (+441), | |
| | 15130 (+47709) | |
| Atxn7l3 | 82305 | (−525) |
| Auh | 92779 (−45201), 92778 (−43991) | |
| Aven | 14105 (+17028), 14107 (+20022) | |
| Axin1 | 111319 | (+2129) |
| Axin2 | 83174 (+853), 83175 (+1280) | |
| Azi2 | 66721 | (+13005) |
| B3galt6 | 32044 | (−402) |
| B3galtl | 39702 | (+368) |
| B3gnt2 | 75639 | (+168113) |
| B630019K06Rik | 123796 (+609), 123797 (+1146) | |
| BC005537 | 90992 | (−729) |
| BC005764 | 71278 | (+18677) |
| BC018507 | 93512 | (−295) |
| BC023829 | 124712 | (−393) |
| BC034090 | 6118 | (+9525) |
| BC055111 | 28067 | (+2930) |
| BC067074 | 95292 | (+292356) |
| BC107364 | 21849 | (+145742) |
| Baalc | 101720 | (+33833) |
| Bag3 | 53303 | (+32297) |
| Bahcc1 | 84315 (+72282), 84327 (+110593) | |
| Bai2 | 29573 | (−33573) |
| Barhl2 | 36181 | (+482683) |
| Basp1 | 101062 (−784), 101059 (+1388) | |
| Batf | 88020 | (−25571) |
| Baz2a | 74305 | (−60) |
| Baz2b | 12041 | (−118876) |
| Bcas1 | 17789 (+52796), 17788 (+53298) | |
| Bcl11b | 89138 | (+507424) |
| Bcl2l11 | 14900 (−70919), 14914 (+1408) | |
| Bcl7c | 53181 | (−4359) |
| Bend6 | 427 | (−231) |
| Bhlha9 | 79499 (−49029), 79500 (−29983) | |
| Bhlhe40 | 44086 | (−177943) |
| Bloc1s4 | 33510 | (+52608) |
| Bmp2 | 15394 | (−411468) |
| Bmp8a | 29056 (−322084), 29005 (−72526) | |
| Bmpr1a | 96994 | (−491) |
| Bmpr2 | 2025 | (−610) |
| Bola3 | 42808 (+335), 42809 (+7332) | |
| Boll | 1789 | (−87) |
| Bora | 99645 | (−130) |
| Bptf | 83029 | (+103878) |
| Brd2 | 112457 (+679), 112446 (+10617) | |
| Brd3 | 10227 | (−303) |
| Brd7 | 57364 | (+51) |
| Brf1 | 89763 | (+15074) |
| Bri3 | 39315 | (+736) |
| Brwd1 | 110080 | (+41533) |
| Btbd1 | 50782 | (−257) |
| Btbd17 | 83423 | (+9318) |
| Btbd2 | 71510 | (−8750) |
| Btbd6 | 89763 | (+8984) |
| Btc | 35399 | (+261823) |
| Btf3l4 | 28307 | (+52132) |
| Btrc | 122720 | (+173971) |
| Bzw1 | 1853 (−89288), 1854 (−88586) | |
| C130057N11Rik | 16200 | (−126047) |
| C1qtnf9 | 98100 | (+3091) |
| C2cd2l | 61509 | (−45) |
| C2cd4c | 71233 | (+18558) |
| C2cd5 | 46249 | (+106157) |
| Cab39 | 3080 | (−24) |
| Cab39l | 98003 | (+115330) |
| Cables1 | 116231 | (−42562) |
| Cabyr | 116303 | (−96962) |
| Cacna1a | 57101 | (+224303) |
| Cacna1d | 96671 | (+319870) |
| Cacna1f | 123715 | (+11275) |
| Cacna1g | 81037 | (+1079) |
| Cacng1 | 83112 (+131259), 83110 (+143818) | |
| Cacng5 | 83141 | (−338398) |
| Cadm4 | 47605 | (+3383) |
| Calb2 | 58190 | (−9503) |
| Calca | 52371 (−9991), 52367 (+4726) | |
| Calcb | 52371 | (−72295) |
| Calcrl | 13183 (−46178), 13182 (−45893) | |
| Calm1 | 88525 | (−14923) |
| Camsap2 | 5111 | (+376) |
| Camta1 | 31658 | (−235) |
| Cand1 | 73729 | (−147) |
| Capn13 | 114705 | (+145746) |
| Capza2 | 40093 | (+906) |
| Car8 | 24897 | (+880669) |
| Carhsp1 | 105900 | (+5465) |
| Casc1 | 46389 | (+86196) |
| Casc3 | 81805 | (−8376) |
| Cass4 | 17813 | (+2909) |
| Casz1 | 31331 (+96556), 31332 (+97481) | |
| Catsper2 | 14532 | (+37) |
| Cbfa2t3 | 59252 | (−1672) |
| Cbx2 | 84107 | (+957) |
| Cbx4 | 84123 | (+29276) |
| Cbx8 | 84123 | (−15976) |
| Ccar1 | 70362 | (−27) |
| Ccdc112 | 117644 | (−182333) |
| Ccdc113 | 57707 | (−26) |
| Ccdc167 | 111926 | (+46865) |
| Ccdc23 | 28775 | (+9375) |
| Ccdc32 | 14354 | (+6081) |
| Ccdc6 | 70718 (+370), 70735 (+83797) | |
| Ccdc64 | 37103 | (+194) |
| Ccdc64b | 110999 | (−8813) |
| Ccdc71l | 85555 (−39047), 85562 (+1990) | |
| Ccdc83 | 51073 | (−79700) |
| Ccdc88a | 75833 (+333), 75834 (+131048) | |
| Cck | 67038 | (+67224) |
| Ccnj | 122311 | (+633) |
| Ccr8 | 66901 | (+6675) |
| Ccsap | 59470 | (+316) |
| Ccser2 | 97043 | (−64625) |
| Cct8 | 109359 | (−53) |
| Cd101 | 22177 (−25901), 22175 (−23137) | |
| Cd180 | 94665 | (+212637) |
| Cd247 | 6884 | (−70) |
| Cd24a | 69427 (+271), 69428 (+604) | |
| Cd274 | 121613 | (+18642) |
| Cd7 | 84481 | (+21500) |
| Cd81 | 53979 | (+3221) |
| Cd83 | 92015 | (+538590) |
| Cd93 | 15804 | (−78749) |
| Cdadc1 | 98003 | (+41525) |
| Cdan1 | 14493 | (−74800) |
| Cdc40 | 69165 | (+177699) |
| Cdc42bpa | 7649 (−537), 7650 (−135) | |
| Cdc42bpg | 120301 | (+327) |
| Cdc42se2 | 77558 | (−103) |
| Cdca4 | 89708 | (−953) |
| Cdh17 | 25239 | (+62479) |
| Cdk17 | 72296 | (+1803) |
| Cdk2ap1 | 38035 | (−56315) |
| Cdk5r1 | 80064 | (+327) |
| Cdkn1a | 111808 (−16697), 111820 (+23162) | |
| Cdkn2aip | 55629 | (−314) |
| Cdkn2b | 27577 | (+11) |
| Cdon | 61056 | (+310) |
| Cdv3 | 65583 | (+32810) |
| Cdyl | 91520 | (+82370) |
| Cebpb | 17525 | (+32) |
| Celf1 | 13335 | (−167) |
| Celf5 | 71769 | (+12001) |
| Celf6 | 62669 | (+158) |
| Celsr3 | 66037 | (+788) |
| Cenpv | 78273 | (−40) |
| Cep120 | 117853 | (−88216) |
| Cep170b | 89682 | (−33515) |
| Cep76 | 118561 | (+28863) |
| Cep78 | 120998 | (−13406) |
| Cers2 | 21679 | (+529) |
| Cgref1 | 33016 | (−4388) |
| Chd2 | 49807 (−16892), 49777 (+34724) | |
| Chd7 | 24960 (−153484), 24984 (+203), | |
| | 25004 (+104971), 25005 (+108758), | |
| | 25030 (+197591), 25061 (+383019) | |
| Chdh | 96635 (−3540), 96671 (+24593) | |
| Chka | 119808 | (−83160) |
| Chmp4b | 16200 | (+8068) |
| Chmp6 | 84216 | (−253063) |
| Chrd | 106690 | (+71603) |
| Chst10 | 821 | (+99) |
| Chst14 | 14354 | (+96815) |
| Cic | 47728 | (−128) |
| Cited2 | 67943 | (−39215) |
| Ckap5 | 13391 | (−19164) |
| Clasp2 | 66317 | (−22232) |
| Clcf1 | 119913 | (+1420) |
| Clcn2 | 106684 | (−1605) |
| Clec16a | 105995 | (+102985) |
| Clic1 | 112655 | (+287) |
| Clic4 | 30355 (−132), 30310 (+106721) | |
| Clic5 | 113142 | (+521526) |
| Clip1 | 37864 | (−323) |
| Clk2 | 21257 | (−1457) |
| Clk3 | 62515 | (−74) |
| Clnk | 33779 (−766803), 33765 (−755566), | |
| | 33741 (−740455), 33731 (−733236) | |
| Clstn1 | 31374 (−44475), 31380 (+203) | |
| Clvs1 | 25004 (−473425), 25005 (−469638), | |
| | 25030 (−380805), 25061 (−195377) | |
| Clybl | 100431 | (−69754) |
| Cmah | 90955 | (+296404) |
| Cmas | 46200 | (+3524) |
| Cmc1 | 66734 (−76439), 66721 (+96576) | |
| Cmip | 58662 (−95547), 58676 (+333), | |
| | 58689 (+30434), 58731 (+122279) | |
| Cmtm4 | 57758 | (−260) |
| Cnih | 97207 | (+62) |
| Cnn3 | 23051 | (+15980) |
| Cnnm2 | 122943 (+1158), 122950 (+57771), | |
| | 122952 (+75919), 122973 (+103921), | |
| | 122974 (+107347) | |
| Cnnm3 | 592 | (+809) |
| Cnnm4 | 589 | (−104) |
| Cnot6 | 76773 (+5766), 76772 (+6690) | |
| Cnot7 | 55454 | (−853) |
| Cnot8 | 77768 | (−57539) |
| Cntn2 | 4789 | (+22023) |
| Cobll1 | 12317 | (−42) |
| Coch | 86020 | (+55) |
| Cog3 | 99322 | (−97) |
| Cog6 | 19630 | (+717630) |
| Col14a1 | 102053 | (−111186) |
| Col17a1 | 123068 | (+41047) |
| Col5a1 | 10259 | (−121335) |
| Col8a2 | 29227 | (+14642) |
| Colgalt2 | 5838 | (+51900) |
| Commd6 | 99761 | (+3077) |
| Cops4 | 35784 | (+51380) |
| Cops5 | 90 | (−1702) |
| Coro2b | 63022 | (+26359) |
| Cox4i1 | 59005 | (+10363) |
| Cox5a | 62466 | (+1145) |
| Cpe | 55896 | (−14695) |
| Cpeb4 | 75933 | (+17995) |
| Cpn1 | 122563 | (+27176) |
| Cpn2 | 107107 | (+79926) |
| Cpne5 | 111823 | (+81256) |
| Cpox | 108727 | (+6627) |
| Cradd | 72508 | (+143746) |
| Cramp1l | 111194 | (−717) |
| Creb3 | 26019 | (−1073) |
| Creb3l1 | 13435 | (+60824) |
| Crebbp | 105722 | (−831) |
| Creld1 | 44295 | (+11105) |
| Crem | 115875 (−35074), 115859 (+37043) | |
| Crispld2 | 58880 | (−80566) |
| Crtc3 | 50610 | (−159) |
| Cry1 | 72054 | (−880) |
| Cryaa | 112146 (+165145), 112151 (+170166) | |
| Cryzl1 | 109662 | (+34071) |
| Csf2ra | 123656 | (+1173) |
| Csnk1d | 84481 | (−26588) |
| Csnk1g3 | 117853 (−29344), 117883 (+82074) | |
| Cspp1 | 90 | (+1611) |
| Csrnp1 | 66851 | (−7694) |
| Csrnp2 | 105255 | (−57) |
| Ctdp1 | 119331 | (−241609) |
| Ctdsp1 | 66790 | (+225) |
| Ctf1 | 53181 | (+556) |
| Ctgf | 68584 | (+40696) |
| Ctif | 119004 | (+328415) |
| Ctla2a | 93257 | (+3530) |
| Ctnnb1 | 66992 | (−3) |
| Ctnnbip1 | 31374 | (+23927) |
| Cttnbp2 | 40133 (−327500), 40132 (−327205) | |
| Ctu2 | 59218 | (+7846) |
| Cul1 | 41303 (+575), 41318 (+105759) | |
| Cul2 | 115875 | (−48599) |
| Cul4a | 54532 (−69), 54533 (+652), | |
| | 54534 (+26777) | |
| Cul5 | 62157 | (−2508) |
| Cxadr | 109216 | (+15558) |
| Cxcr4 | 4524 (−151682), 4523 (−151327), | |
| | 4522 (−151056), 4514 (−147226), | |
| | 4482 (−14132) | |
| Cxxc5 | 116976 (−55780), 117023 (+46496) | |
| Cyb561 | 82727 | (−2397) |
| Cyp17a1 | 122915 | (+52881) |
| Cyp1b1 | 114933 | (+15641) |
| Cyp2r1 | 52367 | (−68659) |
| Cysltr2 | 98981 (+121958), 98980 (+122429) | |
| Cystm1 | 117071 | (−62291) |
| Cyth1 | 84008 (+68087), 83995 (+113015) | |
| Cyth2 | 48916 | (−204) |
| Cytip | 11959 | (−27414) |
| D10Wsu52e | 72076 | (+4286) |
| D16Ertd472e | 109216 | (+259603) |
| D1Ertd622e | 3835 (+1003), 3834 (+1146) | |
| D5Ertd579e | 33510 | (−23093) |
| D630003M21Rik | 16596 | (+24910) |
| D830014E11Rik | 36142 (−116115), 36153 (−78231) | |
| D930028M14Rik | 47705 | (+29157) |
| Dag1 | 65954 | (−16664) |
| Dand5 | 57140 | (+9578) |
| Dapk3 | 71675 (+8936), 71676 (+10434), | |
| | 71677 (+11324), 71679 (+14520) | |
| Daxx | 112386 | (+1443) |
| Dbf4 | 32279 | (−894) |
| Dcaf10 | 26167 | (+1614) |
| Dcbld1 | 69716 | (+493) |
| Dcun1d4 | 34960 | (+11495) |
| Dda1 | 56380 | (+8499) |
| Ddit4 | 70055 | (−17009) |
| Ddost | 30714 | (−35320) |
| Ddx18 | 4179 | (−39705) |
| Ddx59 | 5114 | (+320) |
| Ddx60 | 55887 | (−136733) |
| Dedd2 | 47705 | (+38245) |
| Dek | 92343 | (+37) |
| Dennd2a | 41056 | (+106664) |
| Dennd4c | 27362 | (+2113) |
| Dennd5a | 51971 | (−8577) |
| Dennd6a | 96426 | (+380) |
| Depdc1b | 94939 | (+74455) |
| Derl1 | 102129 | (+196203) |
| Dgat1 | 103231 | (+12070) |
| Dgkd | 3338 | (−116) |
| Dgkz | 13435 | (−13251) |
| Dhcr24 | 28067 | (+53270) |
| Dhx40 | 80575 | (−54597) |
| Diap3 | 99610 | (+462543) |
| Disc1 | 59572 | (+388267) |
| Dkk4 | 54726 | (+26300) |
| Dlgap1 | 114458 | (−983546) |
| Dmbx1 | 28512 | (−23291) |
| Dmxl1 | 117724 | (+135855) |
| Dnahc17 | 84008 (−51196), 83995 (−6268) | |
| Dnajb14 | 23887 | (+3626) |
| Dnajc11 | 31666 | (+904) |
| Dnajc12 | 70409 | (−8904) |
| Dnajc13 | 65638 | (−60156) |
| Dnajc25 | 26766 | (+280) |
| Dnajc3 | 100110 | (+987) |
| Dnase1l1 | 124857 | (+3556) |
| Dner | 3015 | (−14170) |
| Dnmbp | 122563 | (−46988) |
| Dnmt3a | 84644 | (−233387) |
| Dock3 | 65772 | (−38375) |
| Dock8 | 121440 | (+53900) |
| Donson | 109662 (−6139), 109657 (+30732) | |
| Dopey1 | 64864 | (+410) |
| Dot1l | 71557 | (+1085) |
| Dpm1 | 17631 | (−978) |
| Dppa2 | 108167 | (−10407) |
| Dppa4 | 108167 | (+16335) |
| Dpt | 6755 | (−314870) |
| Dpysl4 | 53587 | (+43970) |
| Dqx1 | 42752 | (+123) |
| Drd3 | 107917 | (−64292) |
| Dtl | 8260 | (+149454) |
| Dtnb | 84644 | (+1252) |
| Dtnbp1 | 92081 | (−3047) |
| Dusp1 | 111351 | (−26972) |
| Dusp10 | 8045 (+23153), 8059 (+113062), | |
| | 8060 (+113414) | |
| Dusp14 | 80310 (−832), 80305 (+12203) | |
| Dusp16 | 45819 | (+75737) |
| Dusp26 | 55063 | (−63849) |
| Dusp5 | 123192 (−104881), 123212 (−32065) | |
| Dusp7 | 65703 | (+471) |
| Dvl3 | 106656 (+501), 106657 (+838) | |
| Dync1h1 | 89392 | (−109795) |
| Dynll2 | 80725 (−15444), 80724 (−35) | |
| Dyrk1a | 110004 | (+150909) |
| E030011O05Rik | 65312 | (+31340) |
| E030025P04Rik | 83193 (−109700), 83175 (+222740) | |
| E130304I02Rik | 48500 | (+421) |
| E2f3 | 91064 (+343), 91063 (+883) | |
| Ebf1 | 76366 | (−72983) |
| Ebi3 | 113937 | (+486) |
| Echdc3 | 8724 | (−109715) |
| Ect2l | 67999 | (+96380) |
| Edem3 | 5714 | (−139246) |
| Edil3 | 93882 | (+12376) |
| Ednrb | 99870 | (+192732) |
| Efna5 | 114232 (−212140), 114231 (−201402) | |
| Efna5 | 114239 (−2963), 114232 (+62470), | |
| | 114231 (+73208) | |
| Efs | 97692 | (+723) |
| Egln1 | 59554 | (+27097) |
| Egln3 | 86265 (−439035), 86220 (−255275), | |
| | 86191 (−109417) | |
| Ehd2 | 47047 | (−2262) |
| Eif3k | 48096 | (+8904) |
| Eif4h | 38417 | (+8940) |
| Eif5 | 89562 | (+3773) |
| Eif5b | 787 | (+594) |
| Elac1 | 118855 | (−4689) |
| Elf2 | 19500 (−809), 19499 (−246) | |
| Elf4 | 124426 (−969), 124425 (−158), | |
| | 124424 (+391) | |
| Elk3 | 72296 | (+148456) |
| Elmo1 | 90599 | (+1864) |
| Elmsan1 | 87848 (−67517), 87840 (−26742) | |
| Elovl7 | 94923 | (+403) |
| Emb | 95446 | (−186553) |
| Eml1 | 89292 | (+29804) |
| Emr1 | 114193 | (−42522) |
| En2 | 32869 | (−71576) |
| Enah | 7901 | (−71387) |
| Enoph1 | 35731 | (−97) |
| Enpp1 | 68584 | (+76020) |
| Enpp6 | 55572 | (−245699) |
| Eogt | 43646 | (+42153) |
| Eomes | 66734 (−251825), 66764 (+4148) | |
| Epas1 | 115543 (+25522), 115556 (+35286) | |
| Epc2 | 11695 | (+50) |
| Epdr1 | 90590 | (+101205) |
| Ephb3 | 106690 | (−400025) |
| Epn2 | 78125 | (−18186) |
| Eps15 | 28361 | (+636) |
| Eps15l1 | 56525 (−73), 56504 (+90163) | |
| Ercc3 | 116627 | (−75300) |
| Ergic1 | 111353 | (−12149) |
| Erich1 | 54620 | (−120) |
| Erich2 | 12500 | (+29360) |
| Erv3 | 15232 | (−43388) |
| Esrp1 | 25171 | (−67233) |
| Etos1 | 53339 | (+126073) |
| Ets2 | 110057 (+152348), 110063 (+185876) | |
| Eva1c | 109525 | (−385) |
| Evl | 89292 | (−102100) |
| Exoc6 | 122190 | (−115170) |
| Eya2 | 17111 (+4445), 17112 (+18240) | |
| Ezh2 | 41318 | (+34951) |
| Ezr | 110510 | (−44626) |
| F2r | 94185 | (+54558) |
| F2rl1 | 94185 | (−38661) |
| F3 | 23061 (−7495), 23064 (+13138) | |
| F5 | 6727 | (+39488) |
| F730035P03Rik | 51388 | (−41000) |
| F830045P16Rik | 15056 | (+151123) |
| Faah | 28512 | (+54709) |
| Faim | 65375 | (−21087) |
| Fam101a | 38091 (−134250), 38112 (+49362) | |
| Fam102a | 10847 | (−116) |
| Fam105b | 101163 | (−98) |
| Fam120a | 92404 | (−6851) |
| Fam122a | 121412 | (−220) |
| Fam129a | 5714 | (+44755) |
| Fam129c | 56404 | (−1558) |
| Fam131a | 106677 | (−1617) |
| Fam131b | 41228 | (+46) |
| Fam132b | 3574 | (+14303) |
| Fam13b | 116834 | (−528) |
| Fam151a | 28075 | (+16023) |
| Fam160b1 | 123468 (−5291), 123480 (+7775), | |
| | 123482 (+14339), 123483 (+30232) | |
| Fam161a | 75722 | (+136588) |
| Fam168b | 484 | (+367) |
| Fam171a2 | 82335 | (+9245) |
| Fam174b | 49807 | (−181669) |
| Fam189a2 | 121391 | (−144197) |
| Fam19a4 | 43646 | (−46618) |
| Fam212b | 22468 | (+21623) |
| Fam219b | 62466 | (−15151) |
| Fam222a | 36866 (+15655), 36872 (+48790) | |
| Fam46a | 64849 (−202755), 64825 (+6859), | |
| | 64824 (+7329) | |
| Fam49b | 102684 | (−273676) |
| Fam53a | 33269 | (+133251) |
| Fam53b | 53449 | (−11022) |
| Fam65b | 90955 | (−14824) |
| Fam72a | 4704 | (−2600) |
| Fam76b | 60018 | (+70446) |
| Fam78a | 10714 | (+44925) |
| Fam83d | 16651 (−86636), 16701 (+408) | |
| Fam83h | 103134 | (−5827) |
| Fam84b | 102424 | (−29240) |
| Fam86 | 105854 (−146773), 105844 (−113039), | |
| | 105843 (−112018), 105838 (−96450), | |
| | 105835 (−42) | |
| Farp1 | 100220 | (+274579) |
| Fasl | 6556 | (+37650) |
| Fbrsl1 | 36476 (+31272), 36472 (+71481) | |
| Fbxl14 | 44738 | (+688) |
| Fbxl17 | 114273 | (+139) |
| Fbxl18 | 39157 (−7436), 39156 (−6866), | |
| | 39153 (−5471) | |
| Fbxl21 | 93052 (−19890), 93057 (−16660), | |
| | 93058 (−15262) | |
| Fbxl3 | 99815 | (−194) |
| Fbxo11 | 115774 | (−56925) |
| Fbxo16 | 98430 | (+77107) |
| Fbxo21 | 37174 | (+25544) |
| Fbxo22 | 62228 | (−58846) |
| Fbxo34 | 97317 (+49951), 97318 (+52039) | |
| Fbxo47 | 81591 (−74933), 81585 (−61961), | |
| | 81584 (−59173) | |
| Fbxo6 | 31292 | (−127) |
| Fbxw7 | 20874 | (−241213) |
| Fbxw8 | 37192 | (+85984) |
| Fcgr1 | 21849 (−12595), 21846 (+15778) | |
| Fem1c | 117644 | (+31710) |
| Fermt1 | 15394 | (−194785) |
| Fermt2 | 97188 | (+130083) |
| Fes | 50483 (−11720), 50482 (−11343), | |
| | 50481 (−10858), 50479 (−9638), | |
| | 50477 (−8702), 50476 (−8048), | |
| | 50473 (−6222), 50472 (−5282), | |
| | 50471 (−5100), 50470 (−4632), | |
| | 50469 (−3691), 50468 (−3073), | |
| | 50467 (−2157), 50466 (−1444), | |
| | 50460 (+1991), 50456 (+4043), | |
| | 50455 (+4415), 50454 (+4964), | |
| | 50453 (+5304), 50452 (+5461), | |
| | 50451 (+6122), 50450 (+6814), | |
| | 50449 (+7008), 50448 (+8241) | |
| Fgf9 | 97977 | (−590) |
| Fhad1 | 31088 | (−413) |
| Fkbp4 | 45428 | (−58) |
| Fkbp6 | 38470 | (+63530) |
| Fkrp | 47217 | (−1523) |
| Fli1 | 60770 | (+31652) |
| Flii | 78054 | (−678) |
| Flot1 | 112845 | (−155) |
| Flot2 | 79694 | (+414) |
| Flt1 | 39498 | (+294848) |
| Fmnl3 | 105111 | (+74099) |
| Fndc3a | 98981 (−217153), 98980 (−216682), | |
| | 98943 (+90) | |
| Fndc3b | 18886 (−893), 18885 (−499) | |
| Fnip2 | 20671 (+231535), 20629 (+324127) | |
| Fosb | 47399 | (+1345) |
| Fosl2 | 33164 (−2760), 33165 (−1851) | |
| Foxc1 | 91248 | (−173945) |
| Foxf2 | 91248 | (+6885) |
| Foxk2 | 84514 (+5034), 84519 (+34504) | |
| Foxl2 | 65375 | (+9679) |
| Foxn2 | 115774 | (−318495) |
| Foxn3 | 88439 | (+168522) |
| Foxo1 | 19617 (+931), 19630 (+31256) | |
| Foxo6 | 28836 | (−118484) |
| Foxp4 | 113559 | (−352) |
| Frmd3 | 27209 | (+217793) |
| Frmd4a | 8585 | (+512) |
| Frmd4b | 43727 | (+170316) |
| Frmd8 | 120211 (+41483), 120195 (+78474), | |
| | 120194 (+78862), 120193 (+80132) | |
| Frrs1 | 23011 | (−63953) |
| Fry | 39703 | (−141361) |
| Fsd1l | 26479 | (+306) |
| Ftsjd1 | 58190 | (−40251) |
| Ftsjd2 | 111924 (−22532), 111926 (+9549) | |
| Furin | 50490 (−1487), 50489 (−356), | |
| | 50483 (+3096), 50482 (+3473), | |
| | 50481 (+3958), 50479 (+5178), | |
| | 50477 (+6114), 50476 (+6768), | |
| | 50473 (+8594), 50472 (+9534), | |
| | 50471 (+9716) | |
| Fut8 | 87205 | (+357) |
| Fxr1 | 19130 | (+495) |
| Fzd5 | 2371 | (−36) |
| Fzd6 | 101720 (−39358), 101722 (+317) | |
| Fzd9 | 38470 | (−35231) |
| Gabrr2 | 25612 | (−29264) |
| Gad1 | 12500 | (−23863) |
| Gadd45a | 42295 (−3678), 42288 (+39029) | |
| Gadl1 | 66527 | (−520916) |
| Galk1 | 83633 | (−7446) |
| Galnt1 | 116519 | (+999) |
| Galnt2 | 59486 | (+957) |
| Galnt4 | 72948 | (−50) |
| Gan | 58662 | (+3337) |
| Gata3 | 9087 (+4898), 9078 (+9764), | |
| | 9076 (+11737), 9075 (+11926), | |
| | 9074 (+12534), 9056 (+21995) | |
| Gatsl2 | 38338 | (+67124) |
| Gcat | 103611 | (−27699) |
| Gcsh | 58648 | (−77404) |
| Gfm2 | 94336 | (−24) |
| Gfod1 | 91913 | (+41931) |
| Gfra4 | 15130 | (+88478) |
| Git2 | 36902 | (−51) |
| Gjb2 | 97904 | (+125) |
| Gjd2 | 14137 | (−37431) |
| Glcci1 | 39887 | (+781) |
| Glce | 62956 | (−52144) |
| Glg1 | 58250 | (+109703) |
| Gls2 | 74334 | (+917) |
| Gltp | 36888 | (−2782) |
| Gm10113 | 92261 | (+109906) |
| Gm10146 | 71188 | (−40794) |
| Gm10234 | 43638 | (+292572) |
| Gm10322 | 69980 | (−100888) |
| Gm10335 | 67862 | (−495717) |
| Gm10463 | 33165 (−1453), 33164 (−544) | |
| Gm10517 | 7955 | (−453) |
| Gm10655 | 62815 (+1550), 62776 (+329329) | |
| Gm10699 | 54620 | (−14) |
| Gm10766 | 14832 | (−589) |
| Gm11077 | 46145 (−78377), 46154 (−55022) | |
| Gm11114 | 64774 (−812952), 64811 (−124907), | |
| | 64812 (−111346), 64819 (−26378), | |
| | 64824 (+7022), 64825 (+7492) | |
| Gm11146 | 109179 | (+574310) |
| Gm11444 | 80411 | (+5562) |
| Gm11696 | 83219 | (+266) |
| Gm17669 | 118561 | (+50086) |
| Gm20388 | 58880 | (+1031) |
| Gm20458 | 16943 | (+385) |
| Gm20503 | 26766 | (+242) |
| Gm2178 | 96421 | (+13239) |
| Gm21988 | 78877 | (−161) |
| Gm3086 | 86615 | (−50504) |
| Gm355 | 14914 | (−463762) |
| Gm4968 | 45379 | (+200568) |
| Gm4980 | 51375 | (−33432) |
| Gm5148 | 19362 (−738021), 19352 (−647476), | |
| | 19347 (−554309), 19283 (+83416) | |
| Gm5258 | 3160 | (+16358) |
| Gm5277 | 20551 | (−55923) |
| Gm5475 | 105245 | (+937) |
| Gm5634 | 123797 | (+69476) |
| Gm5784 | 85150 | (−776313) |
| Gm6020 | 123656 | (+43355) |
| Gm6583 | 36675 | (−26475) |
| Gm6658 | 57425 | (−837) |
| Gm711 | 10173 | (−1249) |
| Gm766 | 46225 (−66066), 46200 (+44180) | |
| Gm7854 | 33824 | (+885) |
| Gm8214 | 8006 (−186112), 8004 (−121801) | |
| Gm8225 | 111351 (−7269), 111353 (+6580) | |
| Gm8226 | 64774 (+75846), 64811 (+763891), | |
| | 64812 (+777452), 64819 (+862420) | |
| Gm8765 | 92500 (+403259), 92501 (+403540) | |
| Gm9754 | 37192 | (+4114) |
| Gm9869 | 62776 | (−204664) |
| Gm9912 | 24417 | (−144363) |
| Gm9920 | 102053 | (−82982) |
| Gm9945 | 77217 | (−389) |
| Gm9958 | 35316 | (+22) |
| Gmcl1 | 43101 | (−1) |
| Gmeb1 | 29746 (−9818), 29743 (−4599) | |
| Gna13 | 83219 | (+557) |
| Gnai1 | 32386 (+350143), 32385 (+379568) | |
| Gnal | 118501 (+45829), 118502 (+47696) | |
| Gnaq | 120998 (−134436), 121017 (+539) | |
| Gnat3 | 32383 (−1918), 32385 (+18224), | |
| | 32386 (+47649) | |
| Gng12 | 42274 | (+943) |
| Gng13 | 111246 (−116736), 111252 (−103555), | |
| | 111255 (−52981) | |
| Gng5 | 24342 | (+1054) |
| Gnl3 | 96799 | (−913) |
| Gnpda1 | 117249 | (−9728) |
| Gnpnat1 | 97188 (−11233), 97184 (+5709) | |
| Gnptab | 72162 | (+323) |
| Gns | 73873 | (+54115) |
| Golga4 | 66764 | (−23826) |
| Golm1 | 93206 | (+72509) |
| Gpbp1 | 95067 | (+1168) |
| Gpc1 | 3679 | (+140) |
| Gpd1 | 105165 | (−11672) |
| Gpd2 | 11870 | (−114082) |
| Gpr112 | 124613 | (−4391) |
| Gpr15 | 108727 | (+41889) |
| Gpr158 | 9799 | (+596421) |
| Gpr34 | 123996 | (−111340) |
| Gpsm1 | 10106 | (+659) |
| Gpx4 | 71361 | (+675) |
| Gramd1c | 107942 | (−9989) |
| Grap | 78125 | (−55394) |
| Grb7 | 81694 (+13328), 81695 (+14653), | |
| | 81720 (+44918) | |
| Grik5 | 47683 | (−855) |
| Grlf1 | 47176 | (−328) |
| Grn | 82335 | (+8122) |
| Grsf1 | 35267 (−674), 35266 (−76) | |
| Gsg2 | 79119 | (−36) |
| Gtdc1 | 11509 | (−66839) |
| Gtf3a | 39436 | (−44431) |
| Gtpbp1 | 103713 | (+32752) |
| Gucd1 | 70902 | (+104859) |
| Gyg | 18698 | (−346) |
| Gzmb | 97846 | (−14097) |
| H1foo | 44519 | (+49026) |
| H2-Ke6 | 112423 | (+12) |
| H2-Oa | 112446 | (+18790) |
| H2-T24 | 112891 | (+26710) |
| H3f3b | 83633 | (+7797) |
| Haao | 115317 (−340072), 115302 (−315074) | |
| Hand1 | 77733 | (−79722) |
| Hapln1 | 93882 (−706749), 93897 (+198854) | |
| Hcn1 | 95552 | (+40226) |
| Hcn3 | 21257 | (−3142) |
| Hdac4 | 3605 | (−121873) |
| Hdac4 | 3605 | (+95344) |
| Hdac7 | 104891 | (+44820) |
| Heatr3 | 57330 | (+48) |
| Hectd1 | 86066 (−1144), 86065 (+241) | |
| Hectd3 | 28624 | (+642) |
| Helz | 83110 (+24744), 83112 (+37303) | |
| Hes1 | 107075 (−56585), 107077 (−55562), | |
| | 107085 (−1019), 107107 (+122266) | |
| Hey1 | 18339 (−101121), 18337 (−99877), | |
| | 18336 (−99224), 18327 (−10338), | |
| | 18326 (−2886) | |
| Hhex | 122190 | (+2361) |
| Hhip | 56727 | (+206870) |
| Hif1a | 86987 (−3566), 86991 (+1304), | |
| | 87004 (+35512) | |
| Hip1r | 37900 (+547), 37916 (+25136), | |
| | 37917 (+25591), 37919 (+26244), | |
| | 37920 (+27395), 37922 (+28488), | |
| | 37923 (+28907), 37924 (+29155), | |
| | 37925 (+29550), 37926 (+29804), | |
| | 37927 (+30037), 37928 (+30358), | |
| | 37930 (+31219), 37931 (+31609), | |
| | 37932 (+32128), 37949 (+44161) | |
| Hipk3 | 13951 | (−170) |
| Hist2h2bb | 21846 | (+8439) |
| Hivep2 | 67830 (−180), 67831 (+133), | |
| | 67862 (+60982) | |
| Hk2 | 42699 | (−105612) |
| Hlx | 8060 (+584824), 8059 (+585176), | |
| | 8045 (+675085) | |
| Hmgb1 | 39638 | (−54) |
| Hmgb2 | 55776 | (+960) |
| Hmgxb4 | 56600 | (+30484) |
| Hmx2 | 53378 | (+436) |
| Hnrnpa0 | 93122 | (−456) |
| Hnrnpa3 | 12810 | (+567) |
| Hnrnpd | 35707 | (+205485) |
| Hnrnph3 | 70377 | (+39596) |
| Hnrnpk | 93154 | (+947) |
| Hnrpdl | 35731 | (−675) |
| Hnrpll | 114968 | (+46) |
| Hook3 | 54986 | (−2148) |
| Hormad2 | 74657 | (+179358) |
| Hps1 | 122487 | (−412133) |
| Hps4 | 36674 | (+298) |
| Hpse2 | 122487 | (+196202) |
| Hs2st1 | 24211 | (+162378) |
| Hs3st1 | 33779 (+1019), 33765 (+12256), | |
| | 33741 (+27367), 33731 (+34586) | |
| Hs6st1 | 526 | (−114) |
| Hsd17b4 | 117768 | (−87168) |
| Hsf1 | 103231 | (+22436) |
| Hsf2bp | 112165 | (+160425) |
| Hsf4 | 57808 | (+699) |
| Hspa13 | 108901 | (−95102) |
| Hspa1b | 112614 | (+6630) |
| Hspb3 | 95299 | (+33139) |
| Htra2 | 42752 | (−3396) |
| Ibtk | 64849 | (+214274) |
| Icmt | 31780 | (+15805) |
| Icos | 2158 | (+25755) |
| Idh2 | 50367 | (−60979) |
| Ier2 | 57101 | (+23185) |
| Ier5l | 10487 (+2157), 10486 (+4030) | |
| Iffo2 | 30840 | (−193046) |
| Ifit1 | 122056 | (+21423) |
| Ifne | 27519 | (−59413) |
| Ift140 | 111194 | (−444) |
| Ift172 | 33058 | (+11382) |
| Igf1r | 49644 (−108777), 49648 (−40814), | |
| | 49653 (−14) | |
| Igfbp4 | 81890 | (+1793) |
| Igsf23 | 47518 | (+17363) |
| Igsf3 | 22210 | (+116958) |
| Ikzf3 | 81720 (+54048), 81695 (+84313), | |
| | 81694 (+85638) | |
| Il10 | 4615 | (+44544) |
| Il12rb2 | 42316 | (+108091) |
| Il17ra | 44862 | (+936) |
| Il17rb | 96635 (+3413), 96620 (+14837), | |
| | 96618 (+15373), 96616 (+16673) | |
| Il18bp | 51697 | (+46961) |
| Il1b | 15056 | (−14340) |
| Il1r2 | 940 | (−134444) |
| Il27ra | 57012 | (+14948) |
| Il2rb | 103482 | (+14779) |
| Il3 | 77479 | (−20829) |
| Il4ra | 52817 | (−3779) |
| Il5 | 77316 | (+6031) |
| Il6st | 95165 | (−35432) |
| Il9 | 93058 (−24983), 93057 (−23585), | |
| | 93052 (−20355) | |
| Ilkap | 3574 | (+18050) |
| Impa2 | 118523 | (+636) |
| Impad1 | 24770 | (+13) |
| Impdh2 | 65993 | (−38603) |
| Ina | 122943 (−251931), 122950 (−195318), | |
| | 122952 (−177170), 122973 (−149168), | |
| | 122974 (−145742) | |
| Ino80d | 2242 | (+542) |
| Inpp5f | 53303 | (−55480) |
| Insig1 | 32869 | (+22708) |
| Insl6 | 121578 (+46396), 121574 (+54008) | |
| Ipmk | 70775 (+28402), 70778 (+50474) | |
| Ipo5 | 100185 | (+502) |
| Irf1 | 77316 | (−43189) |
| Irf2 | 55572 | (+1483) |
| Irf2bp2 | 59689 | (−251373) |
| Irf2bpl | 88145 | (−1861) |
| Irf5 | 40369 | (+2288) |
| Irf8 | 59005 | (−57705) |
| Irg1 | 99792 (−64111), 99800 (−17450) | |
| Irgm2 | 77812 | (+24317) |
| Irs2 | 54431 (+2720), 54430 (+3209), | |
| | 54412 (+13167), 54409 (+15922), | |
| | 54395 (+25939), 54392 (+26890), | |
| | 54391 (+27336) | |
| Isoc1 | 118067 | (+447) |
| Itgb6 | 12140 | (−207690) |
| Itgb7 | 105484 | (+25965) |
| Itm2b | 99150 (−9066), 99147 (−3533), | |
| | 99111 (+51159) | |
| Itpk1 | 88842 | (−14920) |
| Itpr1 | 44086 | (+269568) |
| Jag1 | 15557 (−120478), 15549 (−809), | |
| | 15546 (+1095) | |
| Jak1 | 27833 | (−17) |
| Jak2 | 121560 (−95), 121561 (+302), | |
| | 121574 (+19520), 121578 (+27132) | |
| Jarid2 | 92015 | (−407126) |
| Jdp2 | 88020 | (+61682) |
| Jhdm1d | 41029 (−94938), 41011 (−72) | |
| Kank1 | 121440 | (−183546) |
| Kansl1l | 2407 | (−147) |
| Katnal1 | 39588 | (+332021) |
| Kcmf1 | 42567 (−499), 42566 (+25), | |
| | 42565 (+367) | |
| Kcna3 | 22612 | (+472) |
| Kcne3 | 51444 | (−131) |
| Kcnh8 | 113837 (−818874), 113868 (−542871) | |
| Kcnj1 | 60770 | (+115752) |
| Kcnj6 | 110004 | (+276777) |
| Kcnma1 | 96208 | (+903827) |
| Kctd17 | 103470 | (+19270) |
| Kctd6 | 95628 | (+1300) |
| Kdm3b | 116851 (+431), 116852 (+2473), | |
| | 116857 (+7844) | |
| Kdm4c | 27209 | (−20699) |
| Kdm6b | 78729 | (−4447) |
| Keap1 | 60310 (+115), 60291 (+71892) | |
| Kif17 | 30714 | (+7178) |
| Kif19a | 83423 | (+21190) |
| Kif26a | 89642 | (−5802) |
| Kif2a | 94848 | (−923) |
| Kif5c | 11711 | (+481) |
| Kif6 | 113665 | (+566802) |
| Kif7 | 50303 | (−7294) |
| Klc1 | 89594 | (−35954) |
| Klf12 | 99695 (+368034), 99659 (+849636) | |
| Klf13 | 49412 | (−207) |
| Klf2 | 56504 | (+12278) |
| Klf3 | 34286 (−572640), 34353 (−310798), | |
| | 34404 (+822) | |
| Klf4 | 26546 (−869), 26523 (+180431) | |
| Klf5 | 99659 (+1469), 99695 (+483071) | |
| Klhl21 | 31683 | (+1063) |
| Klhl8 | 35989 | (+54811) |
| Kpna1 | 107624 (+1322), 107629 (+44826) | |
| Kpna2 | 83029 (−28708), 83018 (+41831) | |
| Krt78 | 105445 | (−34026) |
| Krt8 | 105445 | (+16029) |
| Krtcap3 | 33058 | (+28026) |
| Ktn1 | 97330 (−14028), 97338 (+194382) | |
| Lama5 | 18143 | (+32) |
| Lamc2 | 5895 | (+128917) |
| Lamp1 | 54534 | (−26737) |
| Lamtor3 | 23887 | (−47225) |
| Larp1 | 77733 (−97195), 77768 (+37550) | |
| Lat2 | 38417 | (−15385) |
| Lbx1 | 122683 | (+10538) |
| Lca5l | 110097 | (+20922) |
| Lclat1 | 114705 | (+145556) |
| Lcor | 122388 | (+576) |
| Ldb2 | 33897 | (+561237) |
| Lemd2 | 111467 | (−107) |
| Lemd3 | 73830 | (+927) |
| Leng9 | 46780 | (−1782) |
| Letmd1 | 105248 (−25263), 105253 (−5300) | |
| Lgi1 | 122221 | (+13854) |
| Lhx1 | 80333 | (−268690) |
| Lif | 74657 | (+3830) |
| Lin7c | 14062 | (+411) |
| Lmf1 | 111246 (+21258), 111252 (+34439), | |
| | 111255 (+85013) | |
| Lmnb1 | 118014 | (+29740) |
| Lmo4 | 24211 (−205486), 24155 (−2793), | |
| | 24153 (−1725), 24150 (+889), | |
| | 24149 (+1765), 24147 (+3258), | |
| | 24144 (+5129), 24142 (+7286), | |
| | 24131 (+13736), 24130 (+14242), | |
| | 24128 (+15590), 24127 (+16663), | |
| | 24126 (+17072), 24125 (+18086) | |
| Lmod3 | 43727 | (−194445) |
| Lnp | 12768 | (+471) |
| Lnx2 | 39457 | (+16405) |
| Loh12cr1 | 45819 | (+75948) |
| Lonrf1 | 55383 | (−4) |
| Lonrf2 | 805 | (+285323) |
| Lor | 21511 (+1717), 21510 (+2165) | |
| Lpgat1 | 8284 | (+464) |
| Lrcol1 | 36472 (+22926), 36476 (+63135) | |
| Lrmp | 46389 | (+3035) |
| Lrp1 | 74252 (−794), 74249 (+16455) | |
| Lrp4 | 13391 | (+69658) |
| Lrrc10b | 120668 | (+7492) |
| Lrrc2 | 66212 | (+2240) |
| Lrrc32 | 51231 | (+40383) |
| Lrrc57 | 14481 | (+30809) |
| Lrrc58 | 107709 | (+887) |
| Lrrc66 | 34960 | (+140008) |
| Lrrc8d | 36142 (+8427), 36153 (+46311) | |
| Ly9 | 7277 | (−29588) |
| Lysmd4 | 49598 | (−109910) |
| Macf1 | 29056 (+19024), 29005 (+268582) | |
| Maea | 33256 | (+9055) |
| Maf | 58532 | (−118) |
| Maff | 103656 (+2419), 103662 (+12261) | |
| Malt1 | 118405 | (+1547) |
| Mamdc2 | 121354 | (+166230) |
| Mamstr | 48875 | (−4528) |
| Mania | 69821 | (+4) |
| Man1a2 | 22140 | (−675) |
| Man1c1 | 30172 | (−67) |
| Man2a2 | 50460 (−16223), 50456 (−14171), | |
| | 50455 (−13799), 50454 (−13250), | |
| | 50453 (−12910), 50452 (−12753), | |
| | 50451 (−12092), 50450 (−11400), | |
| | 50449 (−11206), 50448 (−9973) | |
| Map10 | 59584 | (−98526) |
| Map1lc3b | 59096 (+1038), 59103 (+60158) | |
| Map2k1 | 63217 | (−75) |
| Map2k4 | 78453 | (+107684) |
| Map3k1 | 95112 (−540), 95085 (+121370) | |
| Map3k2 | 116627 | (+1911) |
| Map3k5 | 68241 | (+900) |
| Map4k2 | 120306 (−1076), 120308 (+1268) | |
| Map4k4 | 915 (−134), 940 (+49411) | |
| Map4k5 | 86615 (−19769), 86614 (+53) | |
| Map7 | 68275 | (+1082) |
| Mapk1ip1 | 53575 | (−828) |
| Mapkapk2 | 4615 | (+33178) |
| Mapkapk3 | 65772 | (+19593) |
| Mapkapk5 | 37480 | (−95) |
| March3 | 118014 | (+187995) |
| March6 | 101327 | (−506) |
| March7 | 12041 (−84824), 12049 (+42) | |
| Marcksl1 | 29469 | (+949) |
| Mark3 | 89562 | (−32636) |
| Marveld3 | 58185 | (+44668) |
| Mast4 | 94716 (−32555), 94665 (+428265) | |
| Mbnl1 | 20008 | (+601) |
| Mbp | 119422 (+14106), 119423 (+14523), | |
| | 119445 (+59382) | |
| Mc2r | 118689 | (+81820) |
| Mc5r | 118689 | (+9828) |
| Mcl1 | 21722 | (+7631) |
| Mdh1 | 75607 | (+1009) |
| Mdm1 | 73612 | (−76882) |
| Med13l | 37242 | (+612) |
| Med4 | 99150 | (−115712) |
| Mef2d | 21115 | (+644) |
| Melk | 26105 | (−29539) |
| Men1 | 120302 | (−3564) |
| Mepce | 38809 | (−156) |
| Mesdc1 | 50843 | (−125) |
| Mettl10 | 53449 | (+28123) |
| Mettl16 | 79203 | (−30716) |
| Mettl24 | 69164 (+366), 69165 (+22162) | |
| Mex3b | 50809 | (−199) |
| Mfrp | 61466 | (−12672) |
| Mfsd10 | 33368 | (+62141) |
| Mfsd11 | 83775 | (−678) |
| Mfsd6 | 1512 | (+88255) |
| Mgat4a | 733 | (+11370) |
| Micall1 | 103630 | (+620) |
| Micu2 | 97974 | (−55) |
| Mier1 | 27986 (−860), 27987 (+3749), | |
| | 27988 (+4220) | |
| Mier3 | 95085 | (+1445) |
| Mill2 | 47298 | (−132) |
| Mipep | 98100 | (−13341) |
| Mknk2 | 71510 | (+10472) |
| Mkrn1 | 41056 | (−30741) |
| Mlec | 36999 (+5705), 36997 (+6549) | |
| Mll3 | 32849 | (−438) |
| Mllt10 | 9619 | (−183) |
| Mmp17 | 38256 | (−59734) |
| Mmp25 | 110999 (−6441), 110998 (+170), | |
| | 110989 (+27995), 110988 (+30995) | |
| Moap1 | 88842 | (+23871) |
| Mob3c | 28500 | (−76) |
| Mocs3 | 17631 | (+735) |
| Mon2 | 74044 (+43), 74030 (+88635), | |
| | 74029 (+90447) | |
| Mphosph8 | 97874 | (+108549) |
| Mpnd | 113949 | (+9331) |
| Mppe1 | 118502 (+109798), 118501 (+111665) | |
| Mpzl1 | 6848 | (−62436) |
| Mrc2 | 82684 | (−107737) |
| Mrm1 | 80333 | (+24810) |
| Mroh8 | 16473 | (+464) |
| Mrpl33 | 33086 | (−17537) |
| Mrpl37 | 28122 | (+154342) |
| Mrps28 | 18339 (+155758), 18337 (+157002), | |
| | 18336 (+157655), 18327 (+246541) | |
| Mrps30 | 95552 | (+744546) |
| Mrps6 | 109692 | (+820) |
| Msh4 | 24619 | (+56369) |
| Msh5 | 112655 | (−3631) |
| Msi2 | 80831 (−3064), 80785 (+239094), | |
| | 80781 (+267279) | |
| Msl1 | 81805 | (+1013) |
| Msr1 | 55412 | (+648947) |
| Msrb3 | 73819 | (+3108) |
| Mta2 | 120549 | (−612) |
| Mtap | 27519 | (−197508) |
| Mtfr2 | 68324 | (+222696) |
| Mthfd1l | 67491 (+127020), 67489 (+138855) | |
| Mtif3 | 39457 | (−96396) |
| Mum1 | 71419 | (+937) |
| Mvb12b | 11028 | (+614) |
| Mxi1 | 123123 | (−168982) |
| Myl1 | 2425 | (+48506) |
| Myo15b | 83614 | (+8007) |
| Myo16 | 54365 | (+448115) |
| Myo1b | 1362 | (+406084) |
| Myo1d | 80090 | (−549) |
| Myo3a | 9799 | (−263540) |
| Myo3b | 12482 (+30601), 12488 (+86092) | |
| Myo6 | 64602 | (−97125) |
| Myocd | 78453 | (−410672) |
| Myrf | 120653 | (−3634) |
| Mzt1 | 99645 | (−161) |
| N4bp2l1 | 39720 | (−102) |
| Naa30 | 97385 | (+1325) |
| Naa35 | 93206 | (+17980) |
| Naa38 | 40132 (−6505), 40133 (−6210) | |
| Nab2 | 74271 | (−4874) |
| Nabp1 | 1362 (−31445), 1347 (+3206), | |
| | 1346 (+3563), 1339 (+10951), | |
| | 1337 (+12296) | |
| Naif1 | 10806 | (+670) |
| Nampt | 85562 (−439641), 85585 (+6826) | |
| Nans | 26257 | (−36591) |
| Narg2 | 63742 (−222953), 63759 (−107593), | |
| | 63817 (−69288), 63869 (−33432), | |
| | 63870 (−33081), 63907 (−6717) | |
| Ncald | 101658 | (−140149) |
| Ncl | 3160 | (+19830) |
| Ncoa2 | 203 (−123050), 189 (+471), | |
| | 143 (+145545) | |
| Ncoa3 | 17173 | (−34) |
| Ncoa7 | 68827 | (−1045) |
| Ncor2 | 38164 (−88490), 38112 (+126405) | |
| Ndfip1 | 117249 | (−70244) |
| Neb | 11801 | (−47856) |
| Nedd9 | 91818 | (−149000) |
| Neu1 | 112614 | (+21355) |
| Neu3 | 51388 | (+5929) |
| Neurog3 | 70274 | (+63332) |
| Nfatc1 | 119336 (−765), 119335 (−25), | |
| | 119331 (+1767) | |
| Nfib | 27226 | (−343716) |
| Nfic | 71769 (−43535), 71756 (−3515) | |
| Nfil3 | 92779 (+6211), 92778 (+7421) | |
| Nfix | 57140 | (−39849) |
| Nfrkb | 60677 | (−104490) |
| Nfyc | 28876 | (−60) |
| Ngef | 3284 | (+14467) |
| Nicn1 | 65954 | (−9866) |
| Nin | 86642 (−14498), 86641 (−9211) | |
| Ninj1 | 92417 (+123), 92418 (+593) | |
| Nipal3 | 30382 | (+55196) |
| Nipbl | 100765 | (−125422) |
| Nkd2 | 93562 | (−60291) |
| Nkx1-1 | 33269 | (−62408) |
| Nkx2-3 | 122507 | (−4177) |
| Nlrp3 | 77927 | (−37) |
| Nmnat2 | 5876 (−52901), 5877 (−52214), | |
| | 5895 (+102429) | |
| Nmrk1 | 121181 (−121), 121182 (+7790) | |
| Nmrk2 | 71679 (+3599), 71677 (+6795), | |
| | 71676 (+7685), 71675 (+9183) | |
| Nmt2 | 8430 | (+341) |
| Nnat | 16518 | (−58614) |
| Nnmt | 61919 | (−225200) |
| Noxa1 | 9946 | (−16266) |
| Npas2 | 840 | (+6) |
| Npas3 | 86148 | (−419499) |
| Npepps | 81456 | (−130) |
| Npnt | 23426 | (+202765) |
| Npr3 | 100906 | (−77875) |
| Nr1d2 | 95816 | (+21) |
| Nr2f6 | 56367 | (−855) |
| Nr3c1 | 117442 (−30157), 117432 (−3721), | |
| | 117431 (−3215) | |
| Nr3c2 | 56618 (−2672), 56619 (−2409) | |
| Nr4a2 | 11870 (−9177), 11862 (+390) | |
| Nr4a3 | 26393 (−1791), 26394 (−740) | |
| Nradd | 66161 | (+87335) |
| Nrarp | 9946 | (−69343) |
| Nrd1 | 28336 (+89538), 28338 (+93959) | |
| Nrde2 | 88525 | (−25568) |
| Nrf1 | 40455 | (+664) |
| Nrxn3 | 88222 | (−328191) |
| Nsa2 | 94336 | (−28) |
| Nsd1 | 92905 (+2065), 92906 (+2582) | |
| Nsun7 | 34654 | (+261652) |
| Nt5e | 64886 | (−1479) |
| Ntng2 | 10352 | (−4396) |
| Nubpl | 86106 | (+406812) |
| Nudtl5 | 99189 | (−4528) |
| Nudt3 | 111537 | (−4139) |
| Nudt4 | 72576 | (−1570) |
| Numa1 | 51697 | (+1351) |
| Nup214 | 10714 | (+64422) |
| Nxn | 79460 | (−483) |
| Nxph4 | 74249 | (−70134) |
| Nxt1 | 15804 | (−150289) |
| Nyx | 123996 | (+54639) |
| Oaz1 | 71578 | (−199) |
| Obfc1 | 123043 (+8852), 123038 (+17995) | |
| Oit3 | 69980 | (−73397) |
| Olfr1025-ps1 | 12768 | (−11341550) |
| Olfr1026 | 12768 | (−11346893) |
| Olfr1028 | 12768 | (−11374688) |
| Olfr1029 | 12768 | (−11398836) |
| Olfr1030 | 12768 | (−11402935) |
| Olfr1031 | 12768 | (−11415442) |
| Olfr1257 | 13263 | (+2674) |
| Olfr1258 | 13263 | (−46201) |
| Onecut3 | 71468 | (+1239) |
| Opa1 | 107075 (+429421), 107077 (+430444) | |
| Orc5 | 32592 | (−865670) |
| Os9 | 74129 | (+17004) |
| Osbpl9 | 28338 (+107658), 28336 (+112079) | |
| Osgin2 | 25353 | (−55624) |
| Ostf1 | 121181 | (−40) |
| Otp | 94137 | (−83187) |
| Otud1 | 9775 | (+367) |
| Otx2 | 97366 | (+203347) |
| Pabpc1 | 101499 (−30086), 101493 (−3265) | |
| Pabpc1l | 16906 | (+28587) |
| Pabpc2 | 117442 | (−256108) |
| Pacs1 | 120075 | (−1118) |
| Pacs2 | 89771 | (+472) |
| Pafah1b1 | 79203 (−15444), 79202 (+140) | |
| Paip1 | 95569 (+314), 95570 (+615), | |
| | 95571 (+1098) | |
| Pak6 | 14312 | (+39507) |
| Palld | 55887 | (−31267) |
| Palm3 | 57012 | (+6156) |
| Pan3 | 39498 | (+1002) |
| Pank2 | 15172 | (+68245) |
| Pank4 | 31859 | (−2031) |
| Papd5 | 57336 | (+591) |
| Papss1 | 23417 | (−12087) |
| Pard3 | 59733 | (−16060) |
| Pard3b | 2158 (−624872), 2206 (+28) | |
| Pard6b | 17613 | (+36384) |
| Parp10 | 103173 | (+12390) |
| Parp11 | 45379 | (−19399) |
| Parp8 | 95446 (−8556), 95445 (−346) | |
| Paxbp1 | 109563 (−12109), 109560 (−1197), | |
| | 109552 (+8500), 109547 (+23840), | |
| | 109546 (+24577) | |
| Pbrm1 | 96799 | (−2745) |
| Pbx3 | 11042 | (+128) |
| Pcgf5 | 122084 | (−60357) |
| Pcid2 | 54533 (−930), 54532 (−209) | |
| Pcif1 | 16997 | (+58) |
| Pcnp | 108588 | (+54708) |
| Pcsk7 | 61721 | (+316) |
| Pdcd1 | 3767 | (−22417) |
| Pdcd1lg2 | 121613 | (−24822) |
| Pdcd6ip | 66317 | (−10997) |
| Pde1a | 13070 | (−56965) |
| Pde2a | 51617 | (+27883) |
| Pde4a | 60291 | (+1726) |
| Pde4b | 27900 (+256628), 27930 (+331977) | |
| Pde4d | 95003 | (−131542) |
| Pde4d | 95003 | (+241176) |
| Pde7b | 68324 | (+154563) |
| Pde8a | 50717 | (+416) |
| Pdgfc | 20738 | (+992) |
| Pdia2 | 111319 | (+58270) |
| Pdia3 | 14532 | (−20) |
| Pdia6 | 85112 | (+1127) |
| Pdik1l | 30118 | (+49) |
| Pdk1 | 12603 | (+475) |
| Pdlim1 | 122249 | (−896) |
| Pdp1 | 25239 | (+145801) |
| Pdpr | 58245 (+523), 58250 (+54872) | |
| Pds5b | 39740 | (+702) |
| Pdzd8 | 123565 | (−4459) |
| Peli2 | 97338 | (−262731) |
| Perp | 68055 (+153951), 68056 (+154497), | |
| | 68057 (+155057) | |
| Pex14 | 31332 (+197966), 31331 (+198891) | |
| Pfn2 | 19802 (−944), 19801 (−466) | |
| Pglyrp3 | 21510 (+66394), 21511 (+66842) | |
| Phc1 | 45048 | (−1727) |
| Phc2 | 29324 | (+39206) |
| Phlda3 | 5051 | (+54) |
| Phlpp2 | 58185 | (+48889) |
| Phtf1 | 22360 | (−52395) |
| Phtf2 | 32420 | (−179) |
| Pi4k2b | 34011 | (+630) |
| Pias3 | 21922 | (+427) |
| Piezo1 | 59218 | (+67340) |
| Pigc | 6592 | (−81136) |
| Pik3cg | 85555 | (−130998) |
| Pik3r1 | 94561 | (+113330) |
| Pip4k2a | 9753 | (−174) |
| Pithd1 | 30464 | (−1011) |
| Pitpnb | 36533 (−231979), 36559 (−122701) | |
| Pitpnm2 | 38003 | (+28698) |
| Pkd1l2 | 58648 | (+11508) |
| Pkd2l2 | 116815 | (−28663) |
| Pkn2 | 24090 | (−134887) |
| Pkn3 | 10442 | (−12286) |
| Pla2g4a | 5619 | (−48882) |
| Plac8 | 35784 | (+2556) |
| Plagl2 | 16076 | (−59) |
| Plat | 54756 | (−187) |
| Plau | 96057 | (+34) |
| Plaur | 47593 (−2903), 47596 (+4) | |
| Plcb3 | 120378 | (−1133) |
| Plcg1 | 16730 | (+335) |
| Plcg2 | 58689 (−210838), 58731 (−118993) | |
| Plec | 103173 | (−24708) |
| Plekha5 | 46075 | (+456) |
| Plekhg2 | 47965 | (−5307) |
| Plekhh1 | 87278 | (+459) |
| Plekhm1 | 82468 | (+15792) |
| Plin1 | 50303 | (+11296) |
| Plxdc1 | 81591 (+27312), 81585 (+40284), | |
| | 81584 (+43072) | |
| Plxnc1 | 72508 | (−235773) |
| Plxnd1 | 44522 (−115), 44519 (+1041) | |
| Pm20d2 | 25623 | (−27) |
| Pmepa1 | 17946 | (+476) |
| Pnn | 86417 | (−75) |
| Pofut1 | 16076 | (−48) |
| Polb | 54726 | (+3094) |
| Pold4 | 119913 | (−16241) |
| Poldip3 | 104152 | (−1096) |
| Pole4 | 42654 | (+15842) |
| Poll | 122720 | (+22826) |
| Polr2h | 106684 | (+57) |
| Pou3f1 | 29080 | (+14219) |
| Ppa1 | 70251 | (+6332) |
| Ppa2 | 23520 | (+539) |
| Ppapdc1b | 54918 | (−116322) |
| Pparg | 44451 | (+97334) |
| Ppcdc | 62437 | (+171826) |
| Ppfibp1 | 46595 | (+58796) |
| Ppif | 96375 | (+66615) |
| Ppm1a | 86799 | (+3024) |
| Ppm1h | 74029 (+307276), 74030 (+309088) | |
| Ppp1ca | 119903 | (+4247) |
| Ppp1r12a | 73132 (−510), 73134 (+669) | |
| Ppp1r14a | 48134 | (−187) |
| Ppp1r14b | 120378 | (−4126) |
| Ppp1r16b | 16651 | (+16059) |
| Ppp1r3e | 97682 | (+250) |
| Ppp2r2d | 53575 | (+729) |
| Ppp2r3a | 65524 | (−42) |
| Ppp2r4 | 10486 (+54130), 10487 (+56003) | |
| Ppp2r5a | 8260 | (−29040) |
| Ppp2r5c | 89392 | (+5918) |
| Ppp2r5e | 87037 | (−176) |
| Ppp3cb | 95993 | (−22) |
| Ppp4r4 | 88963 | (+346) |
| Ppt2 | 112553 | (−3977) |
| Pqlc1 | 119255 | (+37) |
| Prc1 | 50419 | (+20142) |
| Prdm10 | 60677 | (+1164) |
| Prdm14 | 143 | (−101375) |
| Prdx6 | 6475 | (+970) |
| Prdx6b | 13070 | (−327074) |
| Prep | 69597 | (+560) |
| Prex1 | 17353 | (−5038) |
| Prickle1 | 104667 | (−638) |
| Prima1 | 88929 | (−92083) |
| Prkaca | 57002 | (+3913) |
| Prkar2b | 85532 | (−53) |
| Prkca | 83155 (−34331), 83141 (+94168) | |
| Prkcd | 96760 | (−17079) |
| Prkcq | 9170 | (−798219) |
| Prl5a1 | 91022 | (+615460) |
| Prnp | 15232 | (−6793) |
| Prob1 | 116952 | (−7) |
| Prom2 | 14868 | (−17994) |
| Prorsd1 | 75834 | (+10327) |
| Prpf40b | 105111 | (+1112) |
| Prph2 | 113406 | (+98704) |
| Prr3 | 112891 | (−13614) |
| Prrt1 | 112553 | (+1592) |
| Prrt3 | 44295 | (+7416) |
| Prx | 47882 | (−8285) |
| Psd2 | 117023 | (−88516) |
| Psma7 | 18124 (−707), 18123 (+20) | |
| Psmg1 | 110080 (−49954), 110063 (+103009), | |
| | 110057 (+136537) | |
| Pspc1 | 97874 | (+1519) |
| Ptbp1 | 71278 | (+1530) |
| Pten | 121936 | (+684) |
| Ptges3 | 74305 | (−24) |
| Ptgfrn | 22177 (+54821), 22175 (+57585) | |
| Ptgr2 | 87840 (−64475), 87848 (−23700) | |
| Ptgs2 | 5619 | (−89952) |
| Ptms | 45188 | (+171) |
| Ptp4a1 | 390 | (+331) |
| Ptplad1 | 63283 | (+79) |
| Ptpn13 | 35938 | (+170453) |
| Ptpn9 | 62411 (+738), 62412 (+1208) | |
| Pura | 117070 (+317), 117071 (+5386) | |
| Pvr | 47518 | (−12220) |
| Pxdc1 | 91387 | (+254453) |
| Qrich1 | 65993 | (+4753) |
| Rab10 | 84594 | (+2236) |
| Rab24 | 92906 (+109616), 92905 (+110133) | |
| Rab28 | 33804 | (−183) |
| Rab2a | 24960 | (+1793) |
| Rab38 | 50985 | (+882843) |
| Rab43 | 43275 | (−907) |
| Rab44 | 111820 (−20915), 111823 (+21485) | |
| Rab8b | 63609 | (−11084) |
| Rabgef1 | 38316 | (+16990) |
| Rad23b | 26523 | (+1992) |
| Rad51d | 80158 | (+3859) |
| Rad9a | 119903 | (+5198) |
| Raf1 | 44483 | (−669) |
| Rai1 | 77976 | (+730) |
| Rala | 90485 | (−3990) |
| Ramp1 | 3533 | (+123) |
| Ramp3 | 75018 (+9079), 75023 (+15541), | |
| | 75025 (+16769), 75026 (+17020), | |
| | 75027 (+17434), 75031 (+20155), | |
| | 75032 (+20818), 75034 (+22027), | |
| | 75035 (+22987), 75049 (+110517) | |
| Ran | 38238 | (+776) |
| Rap1a | 22468 | (+75108) |
| Rap1b | 73612 | (−218970) |
| Rap1gap | 30624 | (+27324) |
| Rapgef2 | 20671 (−191284), 20629 (−98692), | |
| | 20551 (+196603) | |
| Rara | 81848 | (−4304) |
| Rasa2 | 65312 (−89770), 65290 (−2363) | |
| Rasl11a | 39436 | (+59155) |
| Rassf3 | 73873 | (+57045) |
| Rassf5 | 4675 | (+492) |
| Rb1 | 99111 | (−8290) |
| Rbbp6 | 52715 | (+464) |
| Rbbp8 | 116231 | (+163382) |
| Rbfa | 119244 | (+2024) |
| Rbm11 | 108872 | (+778) |
| Rbm34 | 59733 | (−76917) |
| Rbm5 | 65869 | (+35101) |
| Rbms1 | 12162 (−1), 12140 (+81791) | |
| Rbpms | 55137 | (−788) |
| Rcan3 | 30382 | (−5574) |
| Rcc2 | 30908 | (+702) |
| Rccd1 | 50419 | (+9832) |
| Rcor3 | 8336 | (−217) |
| Rcsd1 | 6848 | (+11117) |
| Rdh10 | 247 (−387), 248 (+30) | |
| Rdx | 62088 | (+876) |
| Rec8 | 97760 | (+956) |
| Reep2 | 116852 (−61177), 116857 (−55806) | |
| Reep5 | 116815 (−7345), 116813 (−2450) | |
| Reps1 | 67985 (+73), 67986 (+305), | |
| | 67999 (+23015) | |
| Ret | 44674 (−724), 44673 (+339) | |
| Retsat | 42515 | (+16916) |
| Rev3l | 69021 | (−212) |
| Rffl | 80158 | (−57724) |
| Rft1 | 96760 | (−29459) |
| Rftn1 | 113665 | (+8700) |
| Rgl1 | 5838 | (+173487) |
| Rgma | 49777 | (+131502) |
| Rgr | 97043 | (+15575) |
| Rgs10 | 53290 | (−434) |
| Rgs13 | 5522 | (+99924) |
| Rgs2 | 5522 (−73287), 5496 (+150165) | |
| Rgs9 | 83193 | (+44060) |
| Rhd | 30206 | (+15092) |
| Rif1 | 11768 | (−45) |
| Rilpl2 | 38051 | (+32125) |
| Rims4 | 16883 | (−33260) |
| Ring1 | 112423 | (−3364) |
| Ripk2 | 25353 | (+94135) |
| Rit1 | 21226 | (−29516) |
| Rlf | 28924 | (−28) |
| Rmi1 | 93154 | (−201) |
| Rnf125 | 116416 | (+25939) |
| Rnf138 | 116416 | (−13765) |
| Rnf139 | 102254 | (+186) |
| Rnf144b | 92344 | (+16) |
| Rnf145 | 76366 | (+25370) |
| Rnf157 | 83698 | (−609) |
| Rnf170 | 54986 | (+2004) |
| Rnf19a | 101467 | (−88) |
| Rnf2 | 5695 | (−119) |
| Rnf207 | 31780 | (+5576) |
| Rnf214 | 61721 | (−8) |
| Rnf24 | 15175 (+317), 15172 (+22136) | |
| Rnf38 | 26105 | (−103729) |
| Rnf8 | 111924 | (+23273) |
| Rnpepl1 | 3692 | (−171) |
| Rock2 | 85082 | (+519) |
| Rora | 63742 (+521167), 63759 (+636527), | |
| | 63817 (+674832), 63869 (+710688), | |
| | 63870 (+711039), 63907 (+737403) | |
| Rpl10-ps3 | 61961 | (−131851) |
| Rpl24 | 108588 | (+8756) |
| Rpl31 | 854 | (+371) |
| Rpl7 | 248 (−1142), 247 (−725) | |
| Rplp0 | 37078 | (−3141) |
| Rplp1 | 62921 (+2901), 62920 (+3692), | |
| | 62917 (+4724), 62916 (+5288), | |
| | 62915 (+5758), 62914 (+6164), | |
| | 62913 (+6370), 62872 (+250317), | |
| | 62826 (+534160) | |
| Rpn2 | 16473 | (+26) |
| Rpp40 | 91520 | (+164127) |
| Rprd1b | 16547 (+86109), 16548 (+86471), | |
| | 16549 (+87150), 16550 (+87504), | |
| | 16552 (+88442), 16556 (+93960), | |
| | 16562 (+104572) | |
| Rps19bp1 | 103787 | (+5063) |
| Rps27l | 63609 | (−15297) |
| Rps3a3 | 94939 | (−279792) |
| Rps6 | 27362 | (+106659) |
| Rps6ka1 | 30025 | (+70169) |
| Rpsa-ps10 | 24417 | (+743762) |
| Rptor | 84216 | (+57598) |
| Rragd | 25608 | (+621) |
| Rrbp1 | 15677 | (+952) |
| Rrm2b | 101658 | (+28521) |
| Rsbn1 | 22360 | (+1595) |
| Rsph3b | 110510 | (+121115) |
| Rtfdc1 | 17813 | (−43747) |
| Rtkn2 | 70620 | (+147747) |
| Rtn2 | 47399 | (+26038) |
| Rtp3 | 66212 | (+35928) |
| Rttn | 119641 | (−71444) |
| Rufy2 | 70376 (+253), 70377 (+4056) | |
| Runx3 | 30310 | (+45441) |
| Rwdd3 | 23032 | (+66637) |
| Rxfp2 | 39703 | (+99894) |
| Rxra | 10259 | (+87889) |
| Rxrb | 112423 | (−3768) |
| Ryk | 65563 (+477), 65564 (+25239) | |
| Ryr3 | 14107 (+517345), 14105 (+520339) | |
| Sacs | 98127 | (+100510) |
| Samd1 | 57002 | (−20781) |
| Samd8 | 96140 | (+919) |
| Samsn1 | 108901 | (+47387) |
| Sap30l | 77726 (−94), 77728 (+924) | |
| Sar1a | 70251 | (−25426) |
| Satb1 | 113868 (−350216), 113837 (−74213), | |
| | 113821 (−1140), 113808 (+18721), | |
| | 113774 (+96215), 113760 (+579153) | |
| Sbf2 | 52021 | (+360795) |
| Sbno1 | 38035 | (+14928) |
| Sc4mol | 55896 | (+26057) |
| Sc5d | 61379 | (+55503) |
| Scaf4 | 109507 | (−57) |
| Scaf8 | 110250 | (+810) |
| Scarb1 | 38164 | (+73390) |
| Scgb1a1 | 120568 | (+96696) |
| Scgb3a1 | 76772 (+42423), 76773 (+43347) | |
| Scmh1 | 28836 | (−41314) |
| Scn1b | 48351 | (−649) |
| Scrib | 103134 | (+54429) |
| Scrt1 | 103244 | (−181) |
| Scyl1 | 120211 (−62390), 120195 (−25399), | |
| | 120194 (−25011), 120193 (−23741) | |
| Sdf4 | 32044 | (+169) |
| Sdk1 | 39081 | (+2254) |
| Sdpr | 1337 (+176977), 1339 (+178322), | |
| | 1346 (+185710), 1347 (+186067) | |
| Sec63 | 69363 | (+929) |
| Sel1l | 88339 | (−974456) |
| Sema3f | 65869 | (−25426) |
| Sema4b | 50367 | (−10470) |
| Sema4f | 42699 | (+59703) |
| Senp6 | 64602 | (+1003) |
| 11-Sep | 35502 | (−53227) |
| Serac1 | 110446 | (+15877) |
| Serbp1 | 42316 | (+1118) |
| Serpinb9 | 91329 | (+4649) |
| Serpinb9b | 91329 | (−19517) |
| Serpine1 | 38686 | (−19494) |
| Serpine2 | 2814 | (+181) |
| Sertad1 | 47882 | (+4129) |
| Sesn1 | 69276 | (+78050) |
| Sesn3 | 60018 | (−378139) |
| Set | 10442 | (+4445) |
| Setd2 | 66161 | (+4461) |
| Setd8 | 38051 | (+6311) |
| Sf1 | 120308 | (−21262) |
| Sf3a2 | 71557 | (−41907) |
| Sfmbt2 | 9170 | (+1713) |
| Sfswap | 38256 | (+23249) |
| Sgcg | 98127 | (+19523) |
| Sgip1 | 27900 (−248732), 27930 (−173383) | |
| Sgms2 | 23417 | (−167794) |
| Sgpl1 | 70180 | (−3) |
| Sgsm2 | 79223 | (−276) |
| Sh2b1 | 52911 | (−214) |
| Sh3bp4 | 3447 (+582), 3452 (+60183) | |
| Sh3gl1 | 113949 | (+18103) |
| Sh3glb1 | 24270 | (−3317) |
| Sh3pxd2a | 123043 (−63757), 123038 (−54614) | |
| Sidt2 | 61728 (+8273), 61727 (+9003) | |
| Sik1 | 112165 (−18291), 112161 (−1229), | |
| | 112160 (−965), 112151 (+7693), | |
| | 112146 (+12714) | |
| Sik2 | 62043 | (−1190) |
| Sik3 | 61750 (+7609), 61758 (+34449) | |
| Sin3a | 62412 | (−80223) |
| Sipa1l2 | 59584 (−78582), 59572 (+50248) | |
| Sirt1 | 70409 (−38624), 70408 (−35) | |
| Skap1 | 81310 | (−428) |
| Ski | 31919 | (−11534) |
| Skida1 | 9612 | (+1067) |
| Skil | 19058 (−30), 19059 (+656) | |
| Slain1 | 99870 | (+1213) |
| Slamf7 | 7277 | (+12020) |
| Slc10a1 | 87621 | (+21243) |
| Slc10a6 | 35938 | (+33758) |
| Slc11a2 | 105253 (−40679), 105248 (−20716), | |
| | 105245 (−29) | |
| Slc12a2 | 118056 | (+678) |
| Slc15a1 | 100268 | (+106144) |
| Slc16al2 | 122056 | (+84995) |
| Slc19a2 | 6727 | (−57720) |
| Slc1a3 | 100765 | (+140879) |
| Slc22a23 | 91387 | (−53036) |
| Slc24a2 | 27375 | (+346730) |
| Slc25a24 | 22851 (+102), 22852 (+695), | |
| | 22864 (+92667) | |
| Slc25a25 | 10806 | (+295) |
| Slc25a29 | 89340 | (+40960) |
| Slc25a33 | 31435 | (−1499) |
| Slc25a38 | 66901 | (−11566) |
| Slc25a39 | 82324 | (−560) |
| Slc25a40 | 32279 | (+760) |
| Slc25a51 | 26167 | (+65046) |
| Slc2a1 | 28757 | (+3131) |
| Slc30a5 | 94561 | (−821460) |
| Slc35d1 | 27988 (+95306), 27987 (+95777) | |
| Slc35e2 | 31982 | (+38) |
| Slc35g1 | 122221 | (−117344) |
| Slc36a4 | 60168 | (+431) |
| Slc37a3 | 41029 | (+75980) |
| Slc44a1 | 26454 | (−244195) |
| Slc44a3 | 23051 | (+89823) |
| Slc48a1 | 104891 | (+2592) |
| Slc4a1ap | 33086 | (+69405) |
| Slc4a7 | 95742 | (+707) |
| Slc5a11 | 52743 | (+3598) |
| Slc5a3 | 109692 | (+768) |
| Slc5a9 | 28421 | (+181516) |
| Slc9a6 | 124597 | (+955) |
| Slco2a1 | 65564 | (−148333) |
| Slco2b1 | 51375 | (+47555) |
| Slco3a1 | 49870 | (+129921) |
| Slco6c1 | 3835 (−532818), 3834 (−532675) | |
| Slk | 123068 | (+71028) |
| Slmap | 96421 | (+7102) |
| Slx4ip | 15546 | (+223381) |
| Smad2 | 119091 (+646), 119092 (+846) | |
| Smad7 | 119004 | (+1752) |
| Smagp | 105272 | (−579) |
| Smap1 | 357 | (−120) |
| Smarcd1 | 105165 | (+3556) |
| Smarcd2 | 82787 | (−85) |
| Smc5 | 121354 | (−8215) |
| Smco4 | 60145 | (−11267) |
| Smcr7 | 78054 | (−457) |
| Smg7 | 5877 (−24719), 5876 (−24032) | |
| Smim6 | 83614 | (−42398) |
| Smndc1 | 123212 (−106471), 123192 (−33655) | |
| Smox | 15196 | (+359) |
| Smurf2 | 83018 | (−37240) |
| Snap23 | 14481 | (+10809) |
| Snapc1 | 86991 (−55322), 87004 (−21114) | |
| Snrk | 67120 | (+47524) |
| Snta1 | 16174 | (+4) |
| Sntb2 | 58060 | (+35196) |
| Snx13 | 85658 | (+501) |
| Snx18 | 95299 (−11973), 95292 (+9124) | |
| Snx21 | 16974 (−69), 16975 (+53) | |
| Socs1 | 105995 | (+137187) |
| Socs2 | 72539 | (+787) |
| Socs6 | 119641 | (−6024) |
| Socs7 | 81476 | (+42685) |
| Sod1 | 109479 (−78332), 109486 (−43058) | |
| Son | 109657 | (+10154) |
| Sorl1 | 61379 (−84500), 61371 (+15767), | |
| | 61351 (+89969) | |
| Sos1 | 115026 | (+88596) |
| Sowaha | 77217 | (+497) |
| Sowahb | 35502 | (−40345) |
| Sox4 | 91022 | (+195769) |
| Sp1 | 105535 | (+4952) |
| Sp3 | 12650 | (+695) |
| Sp5 | 12482 (−348024), 12488 (−292533) | |
| Spata1 | 24342 | (−1135) |
| Spata32 | 82414 | (+149) |
| Spata6 | 28421 | (+1418) |
| Spcs3 | 55701 (+2673), 55698 (+5359) | |
| Spen | 31030 | (−2508) |
| Spesp1 | 62956 | (+159429) |
| Spin1 | 92500 (−32777), 92501 (−32496) | |
| Spint2 | 48133 | (+122) |
| Spocd1 | 29573 | (+3870) |
| Sppl3 | 36940 (+781), 36984 (+80302) | |
| Sprtn | 59554 | (+24271) |
| Spry1 | 19283 | (+1036) |
| Sptan1 | 10432 (−225), 10433 (+364) | |
| Sptb | 87136 | (−58) |
| Sptssa | 86265 (+13677), 86220 (+197437), | |
| | 86191 (+343295) | |
| Spty2d1 | 49029 | (+856) |
| Spz1 | 94012 | (+21260) |
| Src | 16518 | (+83052) |
| Srek1 | 94716 | (+407444) |
| Srgap2 | 4704 | (+2058) |
| Sri | 32270 | (−75693) |
| Srl | 105732 | (+67177) |
| Srp9 | 7901 | (−33383) |
| Srrd | 36674 | (−353) |
| Srrm2 | 111037 | (+1676) |
| Srsf1 | 80725 | (−44396) |
| Srsf11 | 24665 | (+73470) |
| Srsf2 | 83775 | (−78) |
| Srsf3 | 111808 | (+41609) |
| Srsf5 | 87621 | (+1156) |
| Srsf6 | 16763 | (+595) |
| Srsf9 | 37039 | (−97) |
| SS18l1 | 18123 (−96), 18124 (+631) | |
| Ssbp3 | 28098 (−75463), 28122 (+1056) | |
| Ssh1 | 36823 (−162), 36822 (+81) | |
| Ssh2 | 79577 | (+377) |
| St8sia1 | 46249 (−29498), 46225 (+93996) | |
| St8sia2 | 49870 | (−411011) |
| Stard3nl | 90590 (−122873), 90502 (+441517) | |
| Stard7 | 14832 | (+392) |
| Stat5b | 82066 | (−580) |
| Steap4 | 32270 | (+20362) |
| Stim1 | 51748 (−511), 51751 (+800) | |
| Stk11 | 71377 | (−3940) |
| Stk24 | 100268 (−19775), 100267 (−133), | |
| | 100220 (+69191) | |
| Stk32c | 53587 | (+58546) |
| Strn4 | 47217 | (+763) |
| Stt3b | 66527 | (−77227) |
| Stx6 | 6118 | (+64512) |
| Stxbp1 | 10923 | (−204) |
| Styx | 97184 | (+31620) |
| Sub1 | 100906 | (+12509) |
| Sucla2 | 99189 (+105), 99191 (+732) | |
| Suclg2 | 43638 | (+104114) |
| Suco | 6592 | (−11389) |
| Sugt1 | 99559 (−57900), 99572 (−45883) | |
| Sun2 | 103718 (−64), 103713 (+18894) | |
| Supt3 | 113142 | (−67054) |
| Surf4 | 10173 | (+1108) |
| Suv420h1 | 119808 | (+1495) |
| Svil | 115988 (−266575), 116001 (+561) | |
| Swap70 | 52021 | (+32424) |
| Syne2 | 87054 | (−663) |
| Synj1 | 109552 (−24735), 109547 (−9395), | |
| | 109546 (−8658), 109542 (−900) | |
| Synj2 | 110446 | (+122461) |
| Synm | 49648 (−152271), 49644 (−84308) | |
| Synrg | 80305 | (+91726) |
| Syp | 123715 | (−20097) |
| Sypl | 85585 | (−126730) |
| Syt7 | 120668 | (+60865) |
| Sytl2 | 51073 | (−35514) |
| Tacc2 | 53339 | (+69095) |
| Tacr1 | 42654 | (+234612) |
| Taf12 | 29746 | (−12705) |
| Taf3 | 9123 | (+56069) |
| Taf4a | 18116 | (−258) |
| Taf6l | 120516 | (−35) |
| Tagln | 61728 (−10646), 61727 (−9916) | |
| Tapt1 | 33897 (−11864), 33895 (−531) | |
| Taz | 124857 | (−4582) |
| Tbc1d1 | 34286 (+71432), 34353 (+333274) | |
| Tbc1d24 | 111095 | (−1361) |
| Tbc1d30 | 73854 | (+182) |
| Tbc1d4 | 99761 | (−28576) |
| Tbc1d5 | 113808 (−634640), 113774 (−557146), | |
| | 113760 (−74208) | |
| Tbc1d7 | 91913 | (−90882) |
| Tbc1d8 | 860 | (+24) |
| Tbca | 94137 | (+3497) |
| Tbck | 23426 | (+63134) |
| Tbl1xr1 | 18782 | (+780) |
| Tbx2 | 80411 | (+12220) |
| Tcam1 | 82787 | (−3691) |
| Tceb2 | 111037 | (+24116) |
| Tec | 34840 | (+37194) |
| Tesc | 37174 | (−25429) |
| Tet3 | 42809 | (+84897) |
| Tex15 | 55096 | (−52853) |
| Tex261 | 42856 | (−161) |
| Tex35 | 6281 | (−272244) |
| Tfpi | 13183 (+3691), 13182 (+3976) | |
| Tgfa | 43009 | (+303) |
| Tgfbr1 | 26376 | (+15733) |
| Tgif2 | 16415 | (+541) |
| Tgm2 | 16591 (−36814), 16562 (+13143), | |
| | 16556 (+23755), 16552 (+29273), | |
| | 16550 (+30211), 16549 (+30565), | |
| | 16548 (+31244), 16547 (+31606) | |
| Tgoln1 | 42515 | (+1609) |
| Th | 53960 | (−68183) |
| Thap4 | 3743 | (+11) |
| Theg | 71233 | (−8129) |
| Thsd7b | 4482 (−666922), 4514 (−533828), | |
| | 4522 (−529998), 4523 (−529727), | |
| | 4524 (−529372) | |
| Tiam1 | 109486 (−202985), 109479 (−167711) | |
| Tigit | 107917 | (−33745) |
| Tiparp | 20165 | (+815) |
| Tjp2 | 121391 | (+49738) |
| Tle1 | 27208 | (−927) |
| Tle3 | 62815 (−2742), 62826 (+6993), | |
| | 62872 (+290836), 62913 (+534783), | |
| | 62914 (+534989), 62915 (+535395), | |
| | 62916 (+535865), 62917 (+536429), | |
| | 62920 (+537461), 62921 (+538252) | |
| Tle4 | 120977 | (−115648) |
| Tlk1 | 12531 | (−207) |
| Tlk2 | 82684 | (+3382) |
| Tlx1 | 122683 | (+76037) |
| Tm2d2 | 54836 | (−57) |
| Tm9sf2 | 100431 | (+4902) |
| Tmc6 | 83908 | (+125007) |
| Tmcc3 | 72440 | (+496) |
| Tmco6 | 117139 | (−561) |
| Tmem11 | 78066 | (+351) |
| Tmem120b | 37767 | (+740) |
| Tmem135 | 50985 | (+25671) |
| Tmem154 | 20874 | (+44741) |
| Tmem17 | 75639 | (+179897) |
| Tmem170b | 91818 | (−103034) |
| Tmem182 | 1095 | (+69596) |
| Tmem184b | 103662 (+43501), 103656 (+53343) | |
| Tmem186 | 105900 | (−28976) |
| Tmem194b | 1512 | (+8502) |
| Tmem201 | 31428 | (−107) |
| Tmem216 | 120678 | (+428) |
| Tmem242 | 110309 (+442831), 110308 (+443448) | |
| Tmem245 | 26623 | (−59) |
| Tmem247 | 115543 (−137919), 115556 (−128155) | |
| Tmem248 | 38316 | (−15593) |
| Tmem260 | 97366 | (+17784) |
| Tmem41b | 51971 | (+17404) |
| Tmem50a | 30206 | (+35396) |
| Tmem57 | 30205 | (−567) |
| Tmem60 | 32420 | (+112) |
| Tmem81 | 4789 | (+14687) |
| Tmprss1bnl | 35235 | (−61297) |
| Tmprss11e | 35235 | (+8156) |
| Tmprss6 | 103482 (−11653), 103470 (+20736) | |
| Tmtc2 | 73080 | (+1404) |
| Tnfaip3 | 68057 (+15282), 68056 (+15842), | |
| | 68055 (+16388) | |
| Tnfaip8 | 117724 (−50699), 117768 (+21466) | |
| Tnfrsf18 | 32059 (+2189), 32063 (+5402), | |
| | 32064 (+5620) | |
| Tnfrsf22 | 54048 | (−17478) |
| Tnfrsf23 | 54048 | (+18733) |
| Tnfrsf9 | 31621 | (+27948) |
| Tnfsf18 | 6556 | (+256190) |
| Tnk2 | 107387 | (−105) |
| Tnpo3 | 40369 | (+80974) |
| Tnrc6c | 83908 | (+1343) |
| Tns4 | 81890 | (+46269) |
| Tob1 | 81004 | (+861) |
| Tom1 | 56600 | (−9846) |
| Tomm20 | 59689 | (+101014) |
| Tomm20l | 86714 | (−94355) |
| Tomm34 | 16906 | (+17065) |
| Topbp1 | 65583 | (+27755) |
| Tor2a | 10911 | (+441) |
| Tox | 24897 | (−367573) |
| Tpbpb | 93257 | (−28248) |
| Tpgs2 | 116568 | (−3204) |
| Tpm4 | 56458 | (+586) |
| Tprn | 9991 | (+426) |
| Trabd | 104517 | (+540) |
| Traf3 | 89524 | (+520) |
| Traf7 | 111131 | (−129) |
| Trak1 | 67019 (+401), 67038 (+130913) | |
| Tram1 | 203 | (+92731) |
| Trappc6b | 86416 | (+45) |
| Trim24 | 40884 | (+809) |
| Trim27 | 90679 | (+766) |
| Trim56 | 38686 | (+24441) |
| Trim62 | 29353 | (+981) |
| Trim67 | 59545 | (+275) |
| Trim8 | 122870 | (−126) |
| Trio | 101264 | (−271) |
| Triobp | 103611 | (+55451) |
| Trip12 | 3015 | (+128913) |
| Trip13 | 93562 | (+29845) |
| Trp53bp2 | 7955 | (+301) |
| Trp53rk | 17353 | (−74897) |
| Trpc5 | 125856 | (+161947) |
| Trps1 | 101872 | (+126712) |
| Trpv4 | 36872 (+41615), 36866 (+74750) | |
| Trub1 | 123480 (−84122), 123482 (−77558), | |
| | 123483 (−61665) | |
| Tsc22d3 | 125744 | (+804) |
| Tsen2 | 44451 | (−25264) |
| Tshz3 | 48520 | (+225) |
| Tspan15 | 70274 | (+34804) |
| Tsr1 | 79223 | (−742) |
| Tssc4 | 53979 | (−13289) |
| Tsx | 125302 | (+54744) |
| Ttbk2 | 14493 | (+44287) |
| Ttc28 | 36533 (+218982), 36559 (+328260) | |
| Ttc39c | 116303 | (+1018) |
| Ttc7 | 115690 | (+1074) |
| Ttc8 | 88427 (−144), 88439 (+304663) | |
| Ttll10 | 32064 (+19033), 32063 (+19251), | |
| | 32059 (+22464) | |
| Ttyh1 | 46765 | (−99) |
| Tusc3 | 55412 | (+10164) |
| Txk | 34840 | (−78485) |
| Txndc9 | 787 | (−1289) |
| Tysnd1 | 70260 | (+19793) |
| Ubac2 | 100311 | (+980) |
| Ubash3b | 61371 (−950468), 61351 (−876266) | |
| Ubc | 38193 | (−1553) |
| Ube2cbp | 64864 | (−2614) |
| Ube2d2a | 116975 (+511), 116976 (+2479) | |
| Ube2d3 | 23606 | (−13) |
| Ube2e3 | 12923 | (+98) |
| Ube2g1 | 79054 | (+1214) |
| Ube2j1 | 25612 | (+2319) |
| Ube2q2 | 62227 (+881), 62228 (+1225) | |
| Ube2r2 | 25868 | (+481) |
| Ube2w | 260 (+152), 259 (+741) | |
| Ubl3 | 39588 | (−43838) |
| Ubr2 | 113406 | (+1369) |
| Ubr4 | 30840 | (+1015) |
| Ubtd2 | 76012 | (+604) |
| Ubtf | 82317 (−358), 82315 (+787) | |
| Ubxn2a | 84772 | (−27) |
| Uchl5 | 5496 | (+76718) |
| Ugcg | 26785 (−29084), 26789 (+499) | |
| Unc119b | 36999 (−17499), 36997 (−16655) | |
| Unc50 | 733 | (+94695) |
| Ung | 36832 | (+543) |
| Upb1 | 70877 (−86418), 70883 (−80187), | |
| | 70902 (+5611) | |
| Upf1 | 56168 | (−190) |
| Upp1 | 75060 | (+493) |
| Urod | 28624 | (−1546) |
| Usf2 | 48316 (−723), 48315 (−74) | |
| Usp12 | 39417 | (−4644) |
| Usp2 | 61466 | (+22149) |
| Usp24 | 28031 | (+386) |
| Usp25 | 109179 | (+13715) |
| Usp6nl | 8724 | (−29985) |
| Ust | 67653 | (−157) |
| Utp11l | 29080 | (+21736) |
| Uts2 | 31621 | (−48959) |
| Uvrag | 51281 | (+45806) |
| Uvssa | 33256 | (−34076) |
| Vamp3 | 31641 | (−112) |
| Vamp4 | 6635 | (−265) |
| Vapa | 114343 | (−101) |
| Vav1 | 114193 | (+37064) |
| Vav3 | 22864 | (−124837) |
| Vcan | 93897 | (+3019) |
| Vdac3 | 54723 | (−278) |
| Vegfc | 55698 (+447106), 55701 (+449792) | |
| Vmn1r238 | 115859 | (−139049) |
| Vps37a | 55454 | (+374) |
| Vps37b | 37972 (−1709), 37949 (+14471), | |
| | 37932 (+26504), 37931 (+27023), | |
| | 37930 (+27413), 37928 (+28274), | |
| | 37927 (+28595), 37926 (+28828), | |
| | 37925 (+29082), 37924 (+29477), | |
| | 37923 (+29725), 37922 (+30144), | |
| | 37920 (+31237), 37919 (+32388), | |
| | 37917 (+33041), 37916 (+33496) | |
| Vps4a | 58060 | (−60275) |
| Wasf2 | 29863 | (−117969) |
| Wbp1l | 122915 | (+20979) |
| Wbp4 | 99572 (−60540), 99559 (−48523) | |
| Wbscr16 | 38338 | (+9939) |
| Wdpcp | 75607 | (−1051) |
| Wdr1 | 33618 | (−790) |
| Wdr45b | 84519 (+64447), 84514 (+93917) | |
| Wdr5b | 107624 (−56553), 107629 (−13049) | |
| Wdr78 | 27986 | (−479) |
| Whsc1 | 33295 | (−157) |
| Whsc1l1 | 54918 | (+1458) |
| Wnk1 | 44805 | (−98) |
| Wnk2 | 92404 | (+173146) |
| Wnt11 | 51281 | (+256493) |
| Wrb | 110097 | (+25928) |
| Wtap | 110718 | (−370) |
| Wwc2 | 55643 | (−570) |
| Xirp1 | 66851 | (+38654) |
| Xpo1 | 75722 | (−106066) |
| Yes1 | 33210 | (+491) |
| Ypel2 | 80575 | (+131414) |
| Ypel4 | 13213 | (−14) |
| Ypel5 | 114620 | (+824) |
| Yrdc | 29118 | (+641) |
| Ythdc2 | 117607 | (+304) |
| Ythdf2 | 29742 | (+79) |
| Ywhab | 16883 (−43017), 16892 (+558) | |
| Ywhaz | 101552 (+875), 101499 (+155479) | |
| Yy1 | 89338 (−65), 89340 (+1950) | |
| Zbed3 | 94171 | (+4424) |
| Zbtb1 | 87097 (−174), 87099 (+941) | |
| Zbtb16 | 61919 | (+5592) |
| Zbtb18 | 7544 | (−2614) |
| Zbtb2 | 67471 (+1332), 67472 (+3075) | |
| Zbtb22 | 112386 | (−5283) |
| Zbtb42 | 89682 | (+9819) |
| Zbtb46 | 18258 | (−73) |
| Zbtb48 | 31683 | (+17795) |
| Zc3h12c | 62110 (−20186), 62107 (−791), | |
| | 62106 (−314) | |
| Zc3h15 | 13095 | (+494) |
| Zc3havl | 40909 (+27399), 40906 (+32662) | |
| Zc3hav1l | 40909 (−27945), 40906 (−22682), | |
| | 40892 (+734) | |
| Zcchc13 | 125302 | (−161375) |
| Zcchc14 | 59103 (+2275), 59096 (+61395) | |
| Zcchc24 | 96387 (−61680), 96375 (+8071) | |
| Zcwpw1 | 38809 | (−979) |
| Zdhhc18 | 29977 (−86), 29975 (+564) | |
| Zdhhc21 | 27226 | (+10649) |
| Zdhhc23 | 107937 (−2159), 107936 (−1337) | |
| Zeb1 | 116031 (+1533), 116032 (+2026) | |
| Zeb2 | 11509 | (+186082) |
| Zfand5 | 121232 | (+698) |
| Zfp125 | 85161 | (−175098) |
| Zfp217 | 17789 (−232354), 17788 (−231852) | |
| Zfp236 | 119492 (−118181), 119445 (+158190), | |
| | 119423 (+203049), 119422 (+203466) | |
| Zfp318 | 113325 | (+849) |
| Zfp326 | 36181 | (+99189) |
| Zfp335 | 17007 | (+423) |
| Zfp358 | 54189 | (+373) |
| Zfp36 | 47965 | (+1715) |
| Zfp36l2 | 115317 (+1085), 115302 (+26083) | |
| Zfp395 | 98430 (−14868), 98438 (+326) | |
| Zfp428 | 47605 | (−21664) |
| Zfp507 | 48500 | (−25) |
| Zfp516 | 119492 (−99964), 119527 (+37277) | |
| Zfp532 | 118405 | (−147720) |
| Zfp574 | 47683 | (−4022) |
| Zfp583 | 46932 | (+42918) |
| Zfp592 | 50689 (−119), 50690 (+214), | |
| | 50691 (+1004) | |
| Zfp64 | 17691 | (−25855) |
| Zfp651 | 67072 | (+353) |
| Zfp652 | 81208 | (+862) |
| Zfp661 | 14868 | (+27633) |
| Zfp664 | 38091 | (+6506) |
| Zfp667 | 46932 | (+1788) |
| Zfp691 | 28757 | (+62353) |
| Zfp740 | 105484 | (+1361) |
| Zfp746 | 41384 | (−59) |
| Zfp777 | 41379 | (−456) |
| Zfyve16 | 94012 | (−24104) |
| Zfyve9 | 28307 | (−57606) |
| Zhx2 | 102129 | (+1550) |
| Zkscan16 | 26748 | (+88) |
| Zmym5 | 97882 (−1112), 97881 (−100) | |
| Zmynd8 | 17112 (+211141), 17111 (+224936) | |
| Zscan10 | 110988 (+13418), 110989 (+16418) | |
| Zswim6 | 94888 | (+313) |
| Zxdc | 43436 | (+873) |
| Zyg11b | 28253 | (−621) |
| Zyx | 41233 | (−643) |

### REFERENCES

Amara, S. G., Jonas, V., Rosenfeld, M. G., Ong, E. S., and Evans, R. M. (1982). Alternative RNA processing in calcitonin gene expression generates mRNAs encoding different polypeptide products. Nature 298, 240-244.
Artis, D., and Spits, H. (2015). The biology of innate lymphoid cells. Nature 517, 293-301. Assas, B. M., Pennock, J. I., and Miyan, J. A. (2014). Calcitonin gene-related peptide is a key neurotransmitter in the neuro-immune axis. Front Neurosci 8, 23.
Bielecki, P., Riesenfeld, S. J., Kowalczyk, M. S., Amezcua Vesely, M. C., Kroehling, L., Yaghoubi, P., Dionne, D., Janet, A., Steach, H. R., McGee, H. M., et al. (2018). Skin inflammation driven by differentiation of quiescent tissue-resident ILCs into a spectrum of pathogenic effectors. bioRxiv.
Biton, M., Haber, A. L., Rogel, N., Burgin, G., Beyaz, S., Schnell, A., Ashenberg, O., Su, C. W., Smillie, C., Shekhar, K., et al. (2018). T Helper Cell Cytokines Modulate Intestinal Stem Cell Renewal and Differentiation. Cell 175, 1307-1320 e1322.
Blei, D. M., Ng, A. Y., and Jordan, M. I. (2003). Latent Dirichlet allocation. J Mach Learn Res 3, 993-1022.
Blondel, V. D., GuillaumeT, J., Lambiotte, R., and Lefebvre, E. (2008). Fast unfolding of communities in large networks. Journal of Statistical Mechanics: Theory and Experiment 2008.
Brandt, E. B., Strait, R. T., Hershko, D., Wang, Q., Muntel, E. E., Scribner, T. A., Zimmermann, N., Finkelman, F. D., and Rothenberg, M. E. (2003). Mast cells are required for experimental oral allergen-induced diarrhea. The Journal of clinical investigation 112, 1666-1677.
Buenrostro, J. D., Giresi, P. G., Zaba, L. C., Chang, H. Y., and Greenleaf, W. J. (2013). Transposition of native chromatin for fast and sensitive epigenomic profiling of open chromatin, DNA-binding proteins and nucleosome position. Nature methods 10, 1213-1218.
Burton, O. T., Medina Tamayo, J., Stranks, A. J., Miller, S Koleoglou, K. J., Weinberg, E. O., and Oettgen, H. C. (2018). IgE promotes type 2 innate lymphoid cells in murine food allergy. Clin Exp Allergy 48, 288-296.
Butler, A., Hoffman, P., Smibert, P., Papalexi, E., and Satija, R. (2018). Integrating single-cell transcriptomic data across different conditions, technologies, and species. Nat Biotechnol 36, 411-420.
Cardoso, V., Chesne, J., Ribeiro, H., Garcia-Cassani, B., Carvalho, T., Bouchery, T., Shah, K., Barbosa-Morais, N. L., Harris, N., and Veiga-Fernandes, H. (2017). Neuronal regulation of type 2 innate lymphoid cells via neuromedin U. Nature 549, 277-281.
Chatterjee, T. K., Moy, J. A., Cai, J. J., Lee, H. C., and Fisher, R. A. (1993). Solubilization and characterization of a guanine nucleotide-sensitive form of the calcitonin gene-related peptide receptor. Mol Pharmacol 43, 167-175.
Chen, C., Grennan, K., Badner, J., Zhang, D., Gershon, E., Jin, L., and Liu, C. (2011). Removing batch effects in analysis of expression microarray data: an evaluation of six batch adjustment methods. PloS one 6, e17238.
Chesne, J., Cardoso, V., and Veiga-Fernandes, H. (2018). Neuro-immune regulation of mucosal physiology. Mucosal Immunol.
Dey, K. K., Hsiao, C. J., and Stephens, M. (2017). Visualizing the structure of RNA-seq expression data using grade of membership models. PLoS genetics 13, e1006599.
Ding, J., Condon, A., and Shah, S. P. (2018). Interpretable dimensionality reduction of single cell transcriptome data with deep generative models. Nature communications 9, 2002.
Ding, J., Shah, S., and Condon, A. (2016). densityCut: an efficient and versatile topological approach for automatic clustering of biological data. Bioinformatics 32, 2567-2576.
Dumais, S. T., Furnas, G. W., Landauer, T. K., and Harshman, R. (1990). Indexing by Latent Semantic Analysis. Journal of the American Society for Information Science 41, 391-407.
duVerle, D. A., Yotsukura, S., Nomura, S., Aburatani, H., and Tsuda, K. (2016). CellTree: an R/bioconductor package to infer the hierarchical structure of cell populations from single-cell RNA-seq data. BMC Bioinformatics 17, 363.
Edvinsson, L. (2018). CGRP Antibodies as Prophylaxis in Migraine. Cell 175, 1719.
Finak, G., McDavid, A., Yajima, M., Deng, J., Gersuk, V., Shalek, A. K., Slichter, C. K., Miller, H. W., McElrath, M. J., Prlic, M., et al. (2015). MAST: a flexible statistical framework for assessing transcriptional changes and characterizing heterogeneity in single-cell RNA sequencing data. Genome Biol 16, 278.
Gerbe, F., Sidot, E., Smyth, D. J., Ohmoto, M., Matsumoto, I., Dardalhon, V., Cesses, P., Gamier, L., Pouzolles, M., Brulin, B., et al. (2016). Intestinal epithelial tuft cells initiate type 2 mucosal immunity to helminth parasites. Nature 529, 226-230.
Gieseck, R. L., 3rd, Wilson, M. S., and Wynn, T. A. (2018). Type 2 immunity in tissue repair and fibrosis. Nature reviews Immunology 18, 62-76.
Godinho-Silva, C., Cardoso, F., and Veiga-Fernandes, H. (2018). Neuro-Immune Cell Units: A New Paradigm in Physiology. Annual review of immunology.
Haber, A. L., Biton, M., Rogel, N., Herbst, R. H., Shekhar, K., Smillie, C., Burgin, G., Delorey, T. M., Howitt, M. R., Katz, Y., et al. (2017). A single-cell survey of the small intestinal epithelium. Nature 551, 333-339.
Haghverdi, L., Lun, A. T. L., Morgan, M. D., and Marioni, J. C. (2018). Batch effects in singlecell RNA-sequencing data are corrected by matching mutual nearest neighbors. Nat Biotechnol 36, 421-427.
Hammad, H., and Lambrecht, B. N. (2015). Barrier Epithelial Cells and the Control of Type 2 Immunity. Immunity 43, 29-40.
Heng, T. S., Painter, M. W., and Immunological Genome Project, C. (2008). The Immunological Genome Project: networks of gene expression in immune cells. Nature immunology 9, 1091-1094.
Hepworth, M. R., Monticelli, L. A., Fung, T. C., Ziegler, C. G., Grunberg, S., Sinha, R., Mantegazza, A. R., Ma, H. L., Crawford, A., Angelosanto, J. M., et al. (2013). Innate lymphoid cells regulate CD4+ T-cell responses to intestinal commensal bacteria. Nature 498, 113-117.
Howitt, M. R., Lavoie, S., Michaud, M., Blum, A. M., Tran, S. V., Weinstock, J. V., Gallini, C. A., Redding, K., Margolskee, R. F., Osborne, L. C., et al. (2016). Tuft cells, taste-chemosensory cells, orchestrate parasite type 2 immunity in the gut. Science 351, 1329-1333.
Huang, Y., Guo, L., Qiu, J., Chen, X., Hu-Li, J., Siebenlist, U., Williamson, P. R., Urban, J. F., Jr., and Paul, W. E. (2015). IL-25-responsive, lineage-negative KLRG1(hi) cells are multipotential ‘inflammatory’ type 2 innate lymphoid cells. Nature immunology 16, 161-169.
Huang, Y., Mao, K., Chen, X., Sun, M. A., Kawabe, T., Li, W., Usher, N., Zhu, J., Urban, J. F., Jr., Paul, W. E., et al. (2018). S1P-dependent interorgan trafficking of group 2 innate lymphoid cells supports host defense. Science 359, 114-119.
Klein, M., Vaeth, M., Scheel, T., Grabbe, S Baumgrass, R., Berberich-Siebelt, F., Bopp, T., Schmitt, E., and Becker, C. (2012). Repression of cyclic adenosine monophosphate upregulation disarms and expands human regulatory T cells. Journal of immunology 188, 1091-1097.
Kloepfer, K. M., Olenec, J. P., Lee, W. M., Liu, G., Vrtis, R. F., Roberg, K. A., Evans, M. D., Gangnon, R. E., Lemanske, R. F., Jr., and Gem, J. E. (2012). Increased H1N1 infection rate in children with asthma. Am J Respir Crit Care Med 185, 1275-1279.
Klose, C. S. N., Mahlakoiv, T., Moeller, J. B., Rankin, L. C., Flamar, A. L., Kabata, H., Monticelli, L. A., Moriyama, S., Putzel, G. G., Rakhilin, N., et al. (2017). The neuropeptide neuromedin U stimulates innate lymphoid cells and type 2 inflammation. Nature 549, 282-286.
Korsunsky, I., Fan, J., Slowikowski, K., Zhang, F., Wei, K., Baglaenko, Y., Brenner, M., Loh, P.-R., and Raychaudhuri, S. (2018). Fast, sensitive, and flexible integration of single cell data with Harmony. bioRxiv, 461954.
Kotas, M. E., and Locksley, R. M. (2018). Why Innate Lymphoid Cells? Immunity 48, 1081-1090.
Langmead, B., and Salzberg, S. L. (2012). Fast gapped-read alignment with Bowtie 2. Nature methods 9, 357-359.
Langmead, B., Trapnell, C., Pop, M., and Salzberg, S. L. (2009). Ultrafast and memory-efficient alignment of short DNA sequences to the human genome. Genome Biol 10, R25.
Levine, J. H., Simonds, E. F., Bendall, S. C., Davis, K. L., Amir el, A. D., Tadmor, M. D., Litvin, O., Fienberg, H. G., Jager, A., Zunder, E. R., et al. (2015). Data-Driven Phenotypic Dissection of AML Reveals Progenitor-like Cells that Correlate with Prognosis. Cell 162, 184-197. Li, B., and Dewey, C. N. (2011). RSEM: accurate transcript quantification from RNA-Seq data with or without a reference genome. BMC Bioinformatics 12, 323.
Lin, Y., Ghazanfar, S., Wang, K., Gagnon-Bartsch, J. A., Lo, K. K., Su, X., Han, Z.-G., Ormerod, J. T., Speed, T. P., Yang, P., et al. (2018). scMerge: Integration of multiple single-cell transcriptomics datasets leveraging stable expression and pseudo-replication. bioRxiv, 393280.
Locksley, R. M. (2010). Asthma and allergic inflammation. Cell 140, 777-783.
Madamanchi, A. (2007). Beta-adrenergic receptor signaling in cardiac function and heart failure. Mcgill J Med 10, 99-104.
Manis, J. P., Tian, M., and Alt, F. W. (2002). Mechanism and control of class-switch recombination. Trends in immunology 23, 31-39.
Martinez, V. G., and O'Driscoll, L. (2015). Neuromedin U: a multifunctional neuropeptide with pleiotropic roles. Clin Chem 61, 471-482.
McLean, C. Y., Bristor, D., Hiller, M., Clarke, S. L., Schaar, B. T., Lowe, C. B., Wenger, A. M., and Bejerano, G. (2010). GREAT improves functional interpretation of cis-regulatory regions. Nat Biotechnol 28, 495-501.
Molofsky, A. B., Savage, A. K., and Locksley, R. M. (2015). Interleukin-33 in Tissue Homeostasis, Injury, and Inflammation. Immunity 42, 1005-1019.
Moriyama, S., Brestoff, J. R., Flamar, A. L., Moeller, J. B., Klose, C. S. N., Rankin, L. C., Yudanin, N. A., Monticelli, L. A., Putzel, G. G., Rodewald, H. R., et al. (2018). beta2-adrenergic receptormediated negative regulation of group 2 innate lymphoid cell responses. Science 359, 1056-1061.
Mowat, A. M., and Agace, W. W. (2014). Regional specialization within the intestinal immune system. Nature reviews Immunology 14, 667-685.
Neill, D. R., Wong, S. H., Bellosi, A., Flynn, R. J., Daly, M., Langford, T. K., Bucks, C., Kane, C. M., Fallon, P. G., Pannell, R., et al. (2010). Nuocytes represent a new innate effector leukocyte that mediates type-2 immunity. Nature 464, 1367-1370.
Nussbaum, J. C., Van Dyken, S. J., von Moltke, J., Cheng, L. E., Mohapatra, A., Molofsky, A. B., Thornton, E. E., Krummel, M. F., Chawla, A., Liang, H. E., et al. (2013). Type 2 innate lymphoid cells control eosinophil homeostasis. Nature 502, 245-248.
Oh-hashi, Y., Shindo, T., Kurihara, Y., Imai, T., Wang, Y., Morita, H., Imai, Y., Kayaba, Y., Nishimatsu, H., Suematsu, Y., et al. (2001). Elevated sympathetic nervous activity in mice deficient in alphaCGRP. Circ Res 89, 983-990.
Painter, M. W., Davis, S., Hardy, R. R., Mathis, D., Benoist, C., and Immunological Genome Project, C. (2011). Transcriptomes of the B and T lineages compared by multiplatform microarray profiling. Journal of immunology 186, 3047-3057.
Picelli, S., Bjorklund, A. K., Faridani, O. R., Sagasser, S., Winberg, G., and Sandberg, R. (2013). Smart-seq2 for sensitive full-length transcriptome profiling in single cells. Nature methods 10, 1096-1098.
Price, A. E., Liang, H. E., Sullivan, B. M., Reinhardt, R. L., Eisley, C. J., Erle, D. J., and Locksley, R. M. (2010). Systemically dispersed innate IL-13-expressing cells in type 2 immunity.
Proceedings of the National Academy of Sciences of the United States of America 107, 11489-11494.
Pulendran, B., and Artis, D. (2012). New paradigms in type 2 immunity. Science 337, 431-435. Ricardo-Gonzalez, R. R., Van Dyken, S. J., Schneider, C., Lee, J., Nussbaum, J. C., Liang, H. E., Vaka, D., Eckalbar, W. L., Molofsky, A. B Erle, D. J., et al. (2018). Tissue signals imprint ILC2 identity with anticipatory function. Nature immunology 19, 1093-1099.
Robinette, M. L., Fuchs, A., Cortez, V. S., Lee, J. S., Wang, Y., Durum, S. K., Gilfillan, S., Colonna, M., and Immunological Genome, C. (2015). Transcriptional programs define molecular characteristics of innate lymphoid cell classes and subsets. Nature immunology 16, 306-317.
Robinson, M. D., McCarthy, D. J., and Smyth, G. K. (2010). edgeR: a Bioconductor package for differential expression analysis of digital gene expression data. Bioinformatics 26, 139-140.
Rodriguez, G., Ross, J. A., Nagy, Z. S., and Kirken, R. A. (2013). Forskolin-inducible cAMP pathway negatively regulates T-cell proliferation by uncoupling the interleukin-2 receptor complex. The Journal of biological chemistry 288, 7137-7146.
Rowe, R. K., and Gill, M. A. (2015). Asthma: the interplay between viral infections and allergic diseases. Immunol Allergy Clin North Am 35, 115-127.
Rydyznski, C. E., Cranert, S. A., Zhou, J. Q., Xu, H., Kleinstein, S. H., Singh, H., and Waggoner, S. N. (2018). Affinity Maturation Is Impaired by Natural Killer Cell Suppression of Germinal Centers. Cell reports 24, 3367-3373 e3364.
Schep, A. N., Wu, B., Buenrostro, J. D., and Greenleaf, W. J. (2017). chromVAR: inferring transcription-factor-associated accessibility from single-cell epigenomic data. Nature methods 14, 975-978.
Schneider, C. A., Rasband, W. S., and Eliceiri, K. W. (2012). NIH Image to Image.” 25 years of image analysis. Nature methods 9, 671-675.
Shao, J., and Sheng, H. (2010). Amphiregulin promotes intestinal epithelial regeneration: roles of intestinal subepithelial myofibroblasts. Endocrinology 151, 3728-3737.
Soneson, C., and Robinson, M. D. (2018). Bias, robustness and scalability in single-cell differential expression analysis. Nature methods 15, 255-261.
Spencer, S. P., Wilhelm, C., Yang, Q., Hall, J. A., Bouladoux, N., Boyd, A., Nutman, T. B., Urban, J. F., Jr., Wang, J., Ramalingam, T. R., et al. (2014). Adaptation of innate lymphoid cells to a micronutrient deficiency promotes type 2 barrier immunity. Science 343, 432-437.
Stork, P. J., and Schmitt, J. M. (2002). Crosstalk between cAMP and MAP kinase signaling in the regulation of cell proliferation. Trends Cell Biol 12, 258-266.
Stuart, T., Butler, A., Hoffman, P., Hafemeister, C., Papalexi, E., Mauck, W. M., Stoeckius, M., Smibert, P., and Satija, R. (2018). Comprehensive integration of single cell data. bioRxiv, 460147.
Sui, P., Wiesner, D. L., Xu, J., Zhang, Y., Lee, J., Van Dyken, S., Lashua, A., Yu, C., Klein, B. S., Locksley, R. M., et al. (2018). Pulmonary neuroendocrine cells amplify allergic asthma responses. Science.
Tanay, A., and Regev, A. (2017). Scaling single-cell genomics from phenomenology to mechanism. Nature 541, 331-338.
Taylor, S., Huang, Y., Mallett, G., Stathopoulou, C., Felizardo, T. C., Sun, M. A., Martin, E. L., Zhu, N., Woodward, E. L., Elias, M. S., et al. (2017). PD-1 regulates KLRG1+ group 2 innate lymphoid cells. The Journal of experimental medicine 214, 1663-1678.
Tordesillas, L., Benin, M. C., and Sampson, H. A. (2017). Immunology of Food Allergy. Immunity 47, 32-50.
Veiga-Fernandes, H., and Mucida, D. (2016). Neuro-Immune Interactions at Barrier Surfaces. Cell 165, 801-811. 23 von Moltke, J., Ji, M., Liang, H. E., and Locksley, R. M. (2016). Tuft-cell-derived IL-25 regulates an intestinal ILC2-epithelial response circuit. Nature 529, 221-225.
Wagner, A., Regev, A., and Yosef, N. (2016). Revealing the vectors of cellular identity with single-cell genomics. Nat Biotechnol 34, 1145-1160.
Wallrapp, A., Riesenfeld, S. J., Burkett, P. R., Abdulnour, R. E., Nyman, J., Dionne, D., Hofree, M., Cuoco, M. S., Rodman, C., Farouq, D., et al. (2017). The neuropeptide NMU amplifies ILC2-driven allergic lung inflammation. Nature 549, 351-356.
Welch, J., Kozareva, V., Ferreira, A., Vanderburg, C., Martin, C., and Macosko, E. (2018). Integrative inference of brain cell similarities and differences from single-cell genomics. bioRxiv, 459891.
Wolock, S. L., Lopez, R., and Klein, A. M. (2018). Scrublet: computational identification of cell doublets in single-cell transcriptomic data. bioRxiv, 357368.
Zeisel, A., Hochgerner, H., Lonnerberg, P., Johnsson, A., Memic, F., van der Zwan, J., Haring, M., Braun, E., Borm, L. E., La Manno, G., et al. (2018). Molecular Architecture of the Mouse Nervous System. Cell 174, 999-1014 e1022.
Zhang, Y., Liu, T., Meyer, C. A., Eeckhoute, J., Johnson, D. S., Bernstein, B. E., Nusbaum, C., Myers, R. M., Brown, M., Li, W., et al. (2008). Model-based analysis of ChIP-Seq (MACS). Genome Biol 9, R137.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of maintaining or inducing homeostasis of intestinal ILC2 cells in a subject at risk for or having aberrant activation and expansion of the intestinal ILC2 cells, comprising:
administering CGRP, or a CGRP receptor agonist, or a combination thereof to the subject.

2. The method of claim 1, wherein the aberrant activation and expansion of the intestinal ILC2 cells is induced by IL-25.

3. The method of claim 1 or 2, wherein the CGRP is administered intravenously, intraperitoneally, intragastrically, or orally.

4. The method of any of claims 1 to 3, wherein the subject has an allergy or history of allergic symptoms.

5. The method of claim 4, wherein the allergy is a food allergy.

6. The method of claim 4 or 5, wherein the allergy is caused by an allergen that induces epithelial cells in the gut to release IL-25.

7. The method of any of claims 1 to 6, wherein CGRP is administered after the subject has contacted or ingested an allergen.

8. The method of any of claims 1 to 7, wherein the CGRP is administered before an inflammatory response.

9. The method of any of claims 1 to 7, wherein CGRP is administered upon detecting an inflammatory response.

10. The method of any of claims 1 to 9, wherein the subject does not have an infection, such as a helminth infection.

11. The method of any of claims 1 to 10, further comprising administering to the subject one or more agents capable of modulating expression, activity or function of one or more genes selected from the group consisting of: Gpr65, Pdcd1, Crem, Egln3, Adora2a, Rgs2, Gna15, Adrb2, Gadd45a, Areg, Hif1a, Dusp1, Pde4b, Cdkn1a, Akap12 and Il5.

12. A method of maintaining or inducing homeostasis of intestinal ILC2 cells in a subject at risk for or having aberrant activation and expansion of the intestinal ILC2 cells, comprising: administering to a subject in need thereof, one or more agents capable of modulating expression, activity or function of one or more genes selected from the group consisting of: Gpr65, Pdcd1, Crem, Egln3, Adora2a, Rgs2, Gna15, Adrb2, Gadd45a, Areg, Hif1a, Dusp1, Pde4b, Cdkn1a, Akap12 and Il5.

13. The method of claim 11 or 12, wherein the method comprises activating a cAMP response module, wherein the cAMP module comprises one or more genes selected from the group consisting of: Adrb2, Adora2a, Pde4b, Akap12, Areg, Crem and Il5.

14. The method of claim 13, wherein the one or more agents comprises an adenylate cyclase activator.

15. The method of claim 14, wherein the agent is forskolin.

16. The method of claim 11 or 12, wherein the one or more agents comprises an agonist of PD-1.

17. The method of claim 11 or 12, wherein the one or more agents comprises an agonist of GPR65.

18. The method of any of claims 12 to 17, wherein the subject has an allergy or history of allergic symptoms.

19. The method of claim 18, wherein the allergy is a food allergy.

20. The method of claim 18 or 19, wherein the allergy is caused by an allergen that induces epithelial cells in the gut to release IL-25.

21. The method of any of claims 12 to 20, wherein the subject does not have an infection, such as a helminth infection.

22. A method of modulating an ILC2 inflammatory response, comprising:
administering to a population of cells comprising ILC2s, mast cells, Th2 cells and/or fibroblasts one or more agents capable of modulating expression, activity or function of one or more biological programs characterized by ILC Topic 2, myeloid cell Topic 1, T cell Topic 5 or stromal cell Topic 4,
wherein ILC Topic 2 comprises one or more genes or polypeptides selected from the group consisting of: Calca, Hs3st1, Areg, Il13, Il4, Ccl1, Hes1, Il17rb, Lgals7, Homer2, Il5, Gata3, Deptor, Ptpn13, Ly6a, Hba-a1, Kcnn4, Ccr4, Rxrg, Sub1, 1700061F12Rik, Cntnap2, AA467197, Ptgir, Il10, Nfkb1, Lmo4, Pparg, Plaur, Il9r, Serpine1, Scel, Bmp7, Neb, Sox8, Lpcat2, Samsn1, Alox5, Gpr65, Abhd17c, Gm20186, Gm973, Epas1, Ccr8, D430036J16Rik, Cd6, Stxbp6, 9230102O04Rik, Furin and Klf5,
wherein myeloid cell Topic 1 comprises one or more genes or polypeptides selected from the group consisting of: Cpa3, Cma1, Mcpt4, Tpsb2, Fcer1a, Hs3st1, Gata2, Cited4, Cyp11a1, Tph1, Furin, Rab27b, Slc45a3, Ccl1, Il13, Il1rl1, Itga2b, Cited2, Fam110c, Creb3l1, Rgs13, Tpsab1, Cyp26a1, Serpinb1a, Slc18a2, Gmpr, Rprm, Ero1l, Il4, Cd200r3, Glul, Kit, Lat, Alox5, Gchfr, mt-Atp6, Lat2, Prss34, Poln, Klk8, 4932438H23Rik, Slc6a13, Avil, Socs2, Smco4, Ier3, Lxn, Gpr171, Adk and Gata1,
wherein T cell Topic 5 comprises one or more genes or polypeptides selected from the group consisting of: 1700061F12Rik, Il13, Scin, Lgmn, Hlf, Smco4, Npnt, Il17rb, Deptor, Gata3, Gm2a, Il6, Il17a, Ltb4r1, Fgl2, Areg, Fbxl21, AA467197, Il1rl1, Me1, Gm5544, Tmem159, Rasgrp4, 1700012B07Rik, 1700113H08Rik, St6galnac5, Il4, Chdh, Slco2b1, Ccr9, Epas1, Grp, Lztfl1, Gm10369, Kif19a, Tenm4, Serpinf1, Gnb2, Ubox5, Plcl1, Rab31, Ffar2, Slx1b, Asb2, Zfp85, Tmsb4x, Hdc, Pxdc1, Heatr1 and Lgals7, and
wherein stromal cell Topic 4 comprises one or more genes or polypeptides selected from the group consisting of: Ccl21a, Cxcl13, Clu, Ccl19, Acta2, Mfge8, Apoe, Tagln, Cxcl1, Cilp, Ccl2, Il33, Cxcl12, Actg2, Serpina3n, Ccl7, Bst1, Serpina1a, Fmod, Grem1, Serpina1b, Slc36a2, Cnn1, Myh11, Art2b, Actc1, AI838599, Serpina1c, Cr2, Gxylt2, Crym, Dclk1, Serpina1d, Myl9, Parm1, Gm16685, Postn, Chrdl1, Colq, Csn2, Prss12, H2-M2, Trf, Sostdc1, Dsc3, Ctgf, Thbs4, Pcdh15, Rtn4r and A230065H16Rik.

23. The method of claim 22, wherein the one or more biological programs are suppressed, whereby an ILC2 inflammatory response is decreased.

24. The method of claim 22 or 23, wherein the one or more agents modulate the expression, activity or function of one or more genes or polypeptides in ILC Topic 2, myeloid cell Topic 1, T cell Topic 5 or stromal cell Topic 4.

25. The method of any of claims 22 to 24, wherein the population of cells is present in the gut of a subject in need thereof.

26. The method of any of claims 22 to 24, wherein the population of cells is an in vitro population of cells.

27. The method of claim 26, wherein the population of cells is an intestinal organoid.

28. The method of any of claims 12 to 27, wherein the one or more agents comprise an antibody, small molecule, small molecule degrader, genetic modifying agent, antibody-like protein scaffold, aptamer, protein, or any combination thereof.

29. The method of claim 28, wherein the genetic modifying agent comprises a CRISPR system, RNAi system, a zinc finger nuclease system, a TALE, or a meganuclease.

30. The method of claim 29, wherein the CRISPR system is a Class I or Class II CRISPR system.

31. The method of claim 30, wherein the Class II system comprises a Class 2, Type II Cas polypeptide.

32. The method of claim 31, wherein the Type II Cas is a Cas9.

33. The method of claim 30, wherein the Class II system comprises a Class 2, Type V Cas polypeptide.

34. The method of claim 33, wherein the Type V Cas is Cas12a or Cas12b.

35. The method of claim 30, wherein the Class II system comprises a Class 2, Type VI Cas polypeptide.

36. The method of claim 35, wherein the Type VI Cas is Cas13a, Cas13b, Cas13c or Cas13d.

37. The method of claim any of claims 30 to 36, wherein the CRISPR system comprises a dCas fused or otherwise linked to a nucleotide deaminase.

38. The method of claim 37, wherein the nucleotide deaminase is a cytidine deaminase or an adenosine deaminase.

39. A method of quantitating a type 2 immune response comprising determining the ILC2 frequency, wherein increased frequency of ILC2s as compared to a control frequency is associated with an increased type 2 immune response.

40. The method of claim 39, further comprising determining the frequency of one or more cells selected from the group consisting of: mast cells, macrophages, neutrophils, and CD11b+CD103+ dendritic cells, wherein increased frequency of mast cells, macrophages and/or neutrophils, and/or decreased frequency of CD11b+CD103+ dendritic cells as compared to a control frequency is associated with an increased type 2 immune response.

41. A method of quantitating a type 2 immune response, comprising:
determining the expression of one or more genes selected from Table 3 or determining the frequency of the cell types expressing the one or more genes selected from Table 3, wherein changes in expression or frequency according to Table 3 is associated with an increased type 2 immune response.

42. The method of claim 41, further comprising determining the expression of:
one or more genes in ILC2s selected from the group consisting of: Hes1, Il13, Lif, Areg and Il4;
one or more genes in mast cells selected from the group consisting of: Mcpt4, Tph1, Mcpt1, Cma1, and Furin;
one or more genes in macrophages selected from the group consisting of: Irf7, Isg15, Irf8, Irf1, Ccl7, Ccl2, Cxcl12, Pf4 and Ccl24; and/or
one or more genes in plasma cells selected from the group consisting of: Ifi27, Ifitm3, Ifnar1, Ighg1 and Ighe,
wherein increased or decreased expression in the cell type according to FIG. 9B or 9C is associated with an increased type 2 immune response.

43. A method of quantitating a type 2 immune response comprising determining the frequency of IL-33⁺PDPN⁺ fibroblasts in a subject having an allergy, wherein increased frequency of IL-33⁺PDPN⁺ fibroblasts is associated with an increased type 2 immune response.

44. The method of claim 1, wherein CGRP is a polypeptide comprising the amino acid sequence of SEQ ID NO: 1 (acdtatcv thrlagllsr sggvvknnfv ptnvgskaf).

45. The method of claim 44, wherein the CGRP sequence is modified to increase stability of the polypeptide.

46. The method of any one of the preceding claims, wherein the intestinal ILCs are KLRG^{Hi} ST2⁻ILCs.

47. The method of any of claims 39 to 43, comprising detecting a type 2 immune response in a subject in need thereof, wherein when an increased type 2 immune response is detected, the subject is treated according to any of claim 1, 11-17, 22-25 or 28-38.

48. The method of any one of the preceding claims, wherein IBD is treated.

49. The method of claim 48, wherein IBD comprises a disease selected from the group consisting of ulcerative colitis (UC), Crohn's Disease, collagenous colitis, lymphocytic colitis, ischemic colitis, diversion colitis, Behcet's syndrome, infective colitis, indeterminate colitis, and other disorders characterized by inflammation of the mucosal layer of the large intestine or colon.

50. A method of screening for one or more agents capable of modulating an ILC2 immune response, comprising:
administering to a population of cells comprising ILC2s, mast cells, Th2 cells and/or fibroblasts one or more agents; and detecting expression, activity or function of one or more biological programs characterized by ILC Topic 2, myeloid cell Topic 1, T cell Topic 5 or stromal cell Topic 4,
wherein ILC Topic 2 comprises one or more genes or polypeptides selected from the group consisting of: Calca, Hs3st1, Areg, Il13, Il4, Ccl1, Hes1, Il17rb, Lgals7, Homer2, Il5, Gata3, Deptor, Ptpn13, Ly6a, Hba-a1, Kcnn4, Ccr4, Rxrg, Sub1, 1700061F12Rik, Cntnap2, AA467197, Ptgir, Il10, Nfkb1, Lmo4, Pparg, Plaur, Il9r, Serpine1, Scel, Bmp7, Neb, Sox8, Lpcat2, Samsn1, Alox5, Gpr65, Abhd17c, Gm20186, Gm973, Epas1, Ccr8, D430036J16Rik, Cd6, Stxbp6, 9230102O04Rik, Furin and Klf5,
wherein myeloid cell Topic 1 comprises one or more genes or polypeptides selected from the group consisting of: Cpa3, Cma1, Mcpt4, Tpsb2, Fcer1a, Hs3st1, Gata2, Cited4, Cyp11a1, Tph1, Furin, Rab27b, Slc45a3, Ccl1, Il13, Il1rl1, Itga2b, Cited2, Fam110c, Creb3l1, Rgs13, Tpsab1, Cyp26a1, Serpinb1a, Slc18a2, Gmpr, Rprm, Ero1l, Il4, Cd200r3, Glul, Kit, Lat, Alox5, Gchfr, mt-Atp6, Lat2, Prss34, Poln, Klk8, 4932438H23Rik, Slc6a13, Avil, Socs2, Smco4, Ier3, Lxn, Gpr171, Adk and Gata1,
wherein T cell Topic 5 comprises one or more genes or polypeptides selected from the group consisting of: 1700061F12Rik, Il13, Scin, Lgmn, Hlf, Smco4, Npnt, Il17rb, Deptor, Gata3, Gm2a, Il6, Il17a, Ltb4r1, Fgl2, Areg, Fbxl21, AA467197, 1r11, Me1, Gm5544, Tmem159, Rasgrp4, 1700012B07Rik, 1700113H08Rik, St6galnac5, Il4, Chdh, Slco2b1, Ccr9, Epas1, Grp, Lztfl1, Gm10369, Kif19a, Tenm4, Serpinf1, Gnb2, Ubox5, Plcl1, Rab31, Ffar2, Slx1b, Asb2, Zfp85, Tmsb4x, Hdc, Pxdc1, Heatr1 and Lgals7, and
wherein stromal cell Topic 4 comprises one or more genes or polypeptides selected from the group consisting of: Ccl21a, Cxcl13, Clu, Ccl19, Acta2, Mfge8, Apoe, Tagln, Cxcl1, Cilp, Ccl2, Il33, Cxcl12, Actg2, Serpina3n, Ccl7, Bst1, Serpina1a, Fmod, Grem1, Serpina1b, Slc36a2, Cnn1, Myh11, Art2b, Actc1, AI838599, Serpina1c, Cr2, Gxylt2, Crym, Dclk1, Serpina1d, Myl9, Parm1, Gm16685, Postn, Chrdl1, Colq, Csn2, Prss12, H2-M2, Trf, Sostdc1, Dsc3, Ctgf, Thbs4, Pcdh15, Rtn4r and A230065H16Rik.
